(19) United States
(12) Patent Application Publication
(10) Pub. No.: US 2023/0141981 A1
(43) Pub. Date: May 11, 2023

(54) NOVEL COMPOUNDS AND COMPOSITION FOR TARGETED THERAPY OF KIDNEY-ASSOCIATED CANCERS

(71) Applicant: SHANGHAI MICURX PHARMACEUTICALS CO., LTD, Shanghai (CN)

(72) Inventors: Mikhail Fedorovich GORDEEV, Foster City, CA (US); Jinqian LIU, Foster City, CA (US)

(21) Appl. No.: 17/794,141

(22) PCT Filed: Jan. 21, 2021
(86) PCT No.: PCT/US2021/014469

§ 371 Date: Jul. 20, 2022

Related U.S. Application Data
(60) Provisional application No. 62/964,080, filed on Jan. 21, 2020.

(51) Int. Cl. C07K 7/62

(52) U.S. Cl. CPC C07K 7/62

(57) ABSTRACT

The present invention provides therapeutic compounds of the following formula I: or pharmaceutically acceptable salts, hydrates, or solvates thereof that are therapeutic or anticancer agents, pharmaceutical compositions containing them, methods for their use, and methods for preparing these compounds.

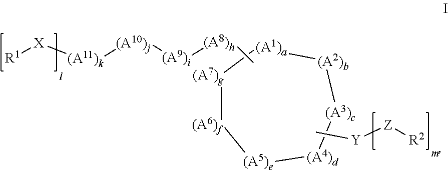

### CROSS-REFERENCE TO RELATED APPLICATIONS

This application is an International Application, which claims the benefit of U.S. Provisional Application No. 62/964,080, filed Jan. 21, 2020, the entirety of which is hereby incorporated by reference for all purposes.

### FIELD OF THE INVENTION

Provided herein are novel active compounds, pharmaceutical compositions thereof, methods for their use, and methods for preparing of the same. These novel agents and compositions thereof possess therapeutic activities useful in therapy of kidney-associated cancers.

### BACKGROUND OF THE INVENTION

Cancers comprise a multi-faceted class of deadly diseases with a profound impact on human lives worldwide. Among these, renal cell carcinoma (RCC) is the most common form of kidney cancer. Globally, RCC is diagnosed in over 200,000 patients annually (as reviewed, for example, by Escudier and Gore in Drugs R&D. 2011, vol. 11, p. 113). This serious disease accounts for about 100,000 deaths annually. Alarmingly, the incidence of RCC is increasing. Thus, 126% increase in incidence and a 36.5% increase in mortality since 1950 was reported for US alone. In particular, metastatic RCC (mRCC) is known to be highly resistant to conventional therapy, with a low 5-year survival rate for a commonly diagnosed stage IV disease of only 0-10% (Motzer et al. N. Engl. J. Med. 1996, vol. 335, pp. 865-75).

A small number of pharmaceutical agents have been developed for treatment of several forms of renal cancers, including renal cell carcinoma (RCC) and metastatic RCC (mRCC), including standard-of-care drugs used to treat renal cancers, such as axitinib and sunitinib. However, anticancer drugs typically exhibit high levels of undesired adverse effects, severely limiting therapeutic utility thereof. These adverse effects could be generally ascribed to cytotoxicity of the anticancer drugs. A cytotoxic mode of activity of chemotherapeutic agents is required for anticancer therapeutic effect thereof. As a result, virtually all anticancer pharmaceuticals are inherently cytotoxic. This toxicity may manifest as adverse effects, including serious adverse effects, with mortality often attributable to chemotherapy. Thus, one of the current standard-of-care drugs for treatment of renal cancer, sunitinib, is known to exhibit high incidence of hematotoxicity (as reported, for example, by Kato et al. in BMC Cancer. 2017, vol. 17, p. 214). This undesired toxicity (also referred to as myelosuppression or a bone marrow toxicity) severely restricts the use of sunitinib in some patient populations, potentially limiting the prescribed dosing regimen required for an optimal anticancer effect (Kato et al. in BMC Cancer. 2017, vol. 17, p. 214). A fatality due to toxicity of such drugs was reported. For example, Prescribing Information for the renal cancer drug axitinib (Inlyta®) includes Warnings on severe hypertension (including hypertensive crisis) and the cardiac failure that has been observed for this drug and can be fatal (as described in Prescribing Information. INLYTA-Axitinib Tablet. June 2020, Pfizer). These adverse effects could be generally ascribed to an off-target action of cytotoxic chemotherapeutic compounds, wherein the inherent in the mode of action toxicity thereof impacts unintended biological compartments, such as bone marrow or heart. Related to such toxicity therapy-induced “bystander killing” of healthy human cells in proximity of cancer cells was also reported (see, for example, by Staudacher and Brown in British Journal of Cancer. 2017, vol. 117, p. 1736).

Therefore, safer anticancer agents are urgently needed. More specifically, novel anti-cancer therapies must offer an improved selectivity with cytotoxic effect thereof targeting only cancerous cells of the affected by disease biological compartments (organs), while leaving healthy tissues and organs minimally affected.

One emerging approach to achieve improved selectivity of anticancer drugs is a targeted delivery of active but toxic agents solely to an organ affected by the disease or, even more specifically, to cancerous cells therein (as reviewed, for example by Tekewe et al. Int. J. Pharm Sci. Res. 2013; Vol. 4, p. 1). In recent years, this urgent need has prompted an emergence of monoclonal antibody drug conjugates (ADCs) that harness innate affinity of antibody(ies) towards the cancer cells, with subsequent release of anticancer drug “payload” directly at the target site (as reviewed, for example, by Cazzamalli et al. in J. Am. Chem. Soc. 2018, vol. 140, p. 1617). However, development of ADCs as viable therapeutics presents several serious challenges, including high cost-of-goods to manufacture, variability in active payload/antibody ratios that require specialized bioanalytical characterization, relatively low chemical stability, excessively long circulation time in vivo, a toxic payload release in unintended biological compartments, and a limited ability of ADCs to penetrate into solid tumors, such as frequently presented in kidney-associated cancers.

Other approaches include efforts to achieve the target drug delivery using non-antibody constructs, such as organic molecule ligands (target-complexing structures), also referred to as small molecule-drug conjugates (SMDCs), typically attempting to utilize molecules capable of recognition of certain targets present in cancer cells, such as folate receptor, prostate-specific membrane antigen, somatostatin receptors and carbonic anhydrase IX (as cited by Cazzamalli et al. in J. Am. Chem. Soc. 2018, vol. 140, p. 1617). However, this approach is limited by serious difficulty in identification of unique small molecules capable of selective recognition of cancer-affected organ(s). Furthermore, majority of such ligands comprise linear peptides generally unstable in vivo due to the rapid metabolism thereof by ubiquitous peptidase enzymes present throughout a body (as reviewed, for example, by Page and Cera in Cell. Mol. Life Sci. 2008, vol. 65, p. 1220).

Provided herein are unique derivatives of acyclic and cyclic peptides (cyclopeptides) particularly suitable for a targeted therapy of various cancers, including kidney cancers.

Various cyclopeptides have been described, for example, in publications WO 2016/083531, WO 2015/149131, WO 2015/135976, US 2015/0031602, WO 2014/188178, WO 2014/108469, CN 103923190, US 2014/0162937, WO 2014/028087, WO 2013/112548, CN 103130876, WO 2013/072695, WO 2012/168820, WO 2012051663, US 2012/0316105, US 2012/0283176, US 2010/0160215, US 2009/0215677, WO 2008/017734, WO 2006/045156, US 2006/0004185, U.S. Pat. Nos. 6,380,356, and 3,450,687. Certain acyclic peptide structures with potential for targeted delivery of active agents have been described, for example in the publications WO 2019136298, US 20180015173, and references cited therein. None of these references specifically describe or generally contemplate the compositions provided herein.

### SUMMARY OF THE INVENTION

Provided herein are novel compounds and composition useful for targeted therapy of cancers, in particular, kidney-associated cancers.

These novel compounds exhibit a surprising ability for targeting kidney tissues and, in particular, cancerous cells therein. This unique affinity of the composition herein to tissues affected by kidney cancers permits to achieve a selective delivery to and accumulation of such molecules at the site of the cancer, with minimal or no accumulation of these therapeutic agents in other healthy tissues.

As a result, a selective and generally safer anticancer therapy is achieved, with significantly minimized adverse effect(s) on other normal organs of a mammal under therapy, as compared to current standard-of-care drugs used to treat kidney cancers, such as axitinib, brivanib, pazopanib, and sunitinib.

In one aspect of this invention, a therapeutic action of compounds herein is achieved by release of one or more of an anticancer element(s) (bioactive payloads and/or drugs) incorporated into such designer molecules. The active payload (drug) may comprise a cytotoxic structure, antibody structure, and/or immunomodulating structure, selected from bioactive structures with ability to kill or inhibit cancer cells, or activate immunomodulating response resulting in similar anticancer action.

Generally, the compounds provided herein are comprised of a peptide, cyclopeptide, or another “target seeker” (ligand) structure with a high affinity (ability to bind) towards kidney cancer and/or kidney cells, along with the active drug(s) substructure, within a single molecule. The active drug(s) (payloads) is(are) connected to a kidney-affinity structure via a framework of uniquely designed linker(s) and spacer(s). This unique design allows for an efficient release of an active drug (payload) directly into kidney cancer cells, or in close proximity thereof, resulting in a targeted anticancer effect.

In another aspect, said composition possesses cytotoxic property(ies) against cancer cells, without a release of an active drug payload (comprised within the administered structure) at the site of a kidney cancer. Upon accumulation at the site of a kidney cancer, such compound(s) kill or inhibit growth of cancer cells directly, and may subsequently break-down into generally non-toxic metabolites.

In yet another aspect, the compositions exhibit modest or no innate anticancer cytotoxicity as intact molecules, but instead accumulate in kidneys and then are metabolized in the organ affected by renal cancers, thereby releasing an anticancer drug (or a cytotoxic agent), at the site of a cancer, to result in anticancer therapeutic effect.

In yet another aspect, the anticancer effect is achieved (upon accumulation at the cancer site) through a combined effect of (i) direct cytotoxic effect of said compound(s), and (ii) a release of an active payload drug comprised within the structure.

In yet another aspect is provided a cyclic peptide conjugate of a tyrosine kinase inhibitor. In some or any embodiments, the cyclic peptide is a polymyxin cyclic peptide as provided herein.

Surprisingly, certain compounds and compositions provided herein are devoid of significant antibiotic and/or other biological activity (such as antibacterial activity), and only exerts the desired cytotoxic effect on kidneys affected by a cancer disease.

Furthermore, while certain composition provided herein incorporates cyclopeptide moieties (structures) of chemical classes generally known to cause renal toxicity (such as polymyxins), the therapeutic compounds of this invention exhibit little or no renal toxicity at the therapeutic dosing levels required for treatment of kidney cancers.

One skilled in art would readily appreciate that not every molecular construct incorporating cytotoxic element(s) (payload) with “heat-seeker” affinity structure (ligand targeting kidney and/or renal cancer cells) with appropriate linkers and deliberately positioned spacers (strategically placed between a ligand and cytotoxic payload) is suitable for use as a therapeutic agent. Surprisingly, compounds and composition provided herein possess a good pharmacological profile, with appropriate stability in blood plasma that precludes premature cytotoxic action, coupled with preferential accumulation thereof in renal cancer cells and/or in kidney affected by a renal cancer(s).

Even more surprisingly, certain compounds provided herein exert their cytotoxic anticancer effect by self-targeted delivery thereof either directly into kidney cancer cells, or only in close proximity of cancer-affected tissues. In part, this composition comprises a class of molecules capable of specifically releasing cytotoxic payloads (incorporated within their structures) as a result of metabolic cleavage by classes of enzymes either specific to or overexpressed (enriched) within the cancer cells (such as cathepsin, glutaminase, and peptide deformylase enzyme (PDF), peptidases, reductases, and similar known enzymes).

In addition to a metabolic degradation by classes of enzymes overexpressed in cancer cells (such as cathepsin, glutaminases, PDF, or similar enzymes) certain compounds provided herein are degraded in vivo through a chemical cleavage, such as pH-dependent self-cleavage known for molecules bearing both a cleavable group (such as an ester, an amide, or a carbamate group) and a free nucleophilic group (such as amine, alcohol, or thiol group). When these two types of cleavable and nucleophilic groups are in certain spacial proximity to each other, and the nucleophile group is essentially free (for example, amine group under neutral, basic, or physiological pH conditions), the nucleophilic group may be acylated by the ester group, resulting in the acyl group transfer onto the nucleophilic atom (such as nitrogen atom in amine group). In another scenario, the free amine may activate an adjacent to the carbamate group amide functionality, leading to the carbamate reaction with the latter, to result in a conversion of the native amide into a bis-acylated imide group. In some composition herein, the cleavage of a chemical designer linker takes place after initial enzymatic metabolism of an auxiliary enzyme-cleavable linker (e.g., peptide substructure, or similar linkers), to overall effect of the release of a cytotoxic payload at the caner target.

In one aspect, provided herein is a compound of formula I:

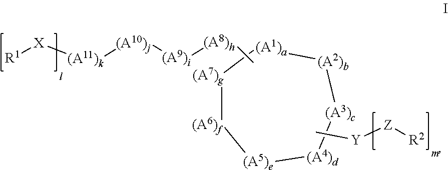

or a pharmaceutically acceptable salt, solvate, or hydrate thereof wherein:

R¹ and R² are optional groups, with at least one of the groups R¹ and R² being present in the formula I; and

R¹ and R² are independently selected from alkyl, aryl, biaryl, heteroaryl, heteroarylaryl, and arylheteroaryl; or

R¹ and R² are groups independently attached to X and Z, respectively, by subtracting a single or multiple H atom(s) from respective parent (precursor) structure(s) (H)ₙR¹ and (H)ₒR² at any one of the following optional H-containing group(s) independently selected from NH, OH, SH, C(═O)OH, CONH, SO₂NH, and S(═O)NH when present in (H)ₙR¹ and (H)ₒR²; and wherein

a) (H)ₙR¹ and (H)ₒR² are independently compound(s) possessing biological or therapeutic activity; or

b) (H)ₙR¹ and (H)ₒR² are independently a cytotoxic compound(s), an antibody(ies), or an immunomodulating compound(s) possessing an activity or capable of inducing an activity against one or more cancer cells, including compounds with activity against one or more renal cancer cells; or

c) (H)ₙR¹ and (H)ₒR² are independently mono- or multi-valent antibody(ies) with activity against one or more cancer cells; or

d) (H)ₙR¹ and (H)ₒR² are independently afatinib ((E)-N-[4-(3-chloro-4-fluoroanilino)-7-[(3S)-oxolan-3-yl]oxyquinazolin-6-yl]-4-(dimethylamino)but-2-enamide), ARS-1630 (same as (R)-1-(4-(6-chloro-8-fluoro-7-(2-fluoro-6-hydroxyphenyl)quinazolin-4-yl)piperazin-1-yl)prop-2-en-1-one), axitinib (same as N-methyl-2-[[3-[(E)-2-pyridin-2-ylethenyl]-1H-indazol-6-yl]sulfanyl]benzamide), BGB-324 (same as 1-(6,7-dihydro-5H-benzo[2,3]cyclohepta[2,4-d]pyridazin-3-yl)-3-N-[(7S)-7-pyrrolidin-1-yl-6,7,8,9-tetrahydro-5H-benzo[7]annulen-3-yl]-1,2,4-triazole-3,5-diamine), BLU-554 (same as N-[(3S,4S)-3-[[6-(2,6-dichloro-3,5-dimethoxyphenyl)quinazolin-2-yl]amino]oxan-4-yl]prop-2-enamide), brivanib (same as (S)—(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate), (R)-1-((4-((4-fluoro-2-methyl-1H-indol-5-yl)oxy)-5-methylpyrrolo[2,1-f][1,2,4]triazin-6-yl)oxy)propan-2-ol, cabozantinib, cediranib, ceritinib, ciforadenant, derazantinib, dovitinib (same as 4-amino-5-fluoro-3-(6-(4-methylpiperazin-1-yl)-1H-benzo[d]imidazol-2-yl)quinolin-2(1H)-one), E-7046, (same as 4-[(1S)-1-[[3-(difluoromethyl)-1-methyl-5-[3-(trifluoromethyl)phenoxy]pyrazole-4-carbonyl]amino]ethyl]benzoic acid), emtansine, englerin (same as (1R,3aR,4S,5R,7R,8S,8aR)-5-(glycoloyloxy)-7-isopropyl-1,4-dimethyldecahydro-4,7-epoxyazulen-8-yl (2E)-3-phenylacrylate), foretinib, lenvatinib (same as 4-[3-chloro-4-(cyclopropylcarbamoylamino)phenoxy]-7-methoxyquinoline-6-carboxamide), monomethyl auristatin E (same as (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide), irinotecan, maytansinoid, neratinib, nilotinib, nintedanib, ozogamicin, paclitaxel, pazopanib (same as 5-[[4-[(2,3-dimethylindazol-6-yl)-methylamino]pyrimidin-2-yl]amino]-2-methylbenzenesulfonamide), regorafenib, sacituzumab, selpercatinib, semaxanib (same as (Z)-3-((3,5-dimethyl-1H-pyrrol-2-yl)methylene)indolin-2-one), sorafenib (same as 4-[4-[[4-chloro-3-(trifluoromethyl)phenyl]carbamoylamino]phenoxy]-N-methylpyridine-2-carboxamide), sunitinib (same as (Z)—N-(2-(diethylamino)ethyl)-5-((5-fluoro-2-oxoindolin-3-ylidene)methyl)-2,4-dimethyl-1H-pyrrole-3-carboxamide), SN38 (same as 7-ethyl-10-hydroxy-camptothecin), sorafenib (same as 4-[4-[[4-chloro-3-(trifluoromethyl)phenyl]carbamoylamino], phenoxy]-N-methyl-pyridine-2-carboxamide), sunitinib (same as (Z)—N-(2-(diethylamino)ethyl)-5-((5-fluoro-2-oxoindolin-3-ylidene)methyl)-2,4-dimethyl-1H-pyrrole-3-carboxamide), trastuzumab, tesirine (same as [4-[[(2S)-2-[[(2S)-2-[3-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]propanoylamino]-3-methylbutanoyl]amino]propanoyl]amino]phenyl]methyl (6S,6aS)-3-[5-[[(6aS)-2-methoxy-8-methyl-11-oxo-6a,7-dihydropyrrolo[2,1-c][1,4]benzodiazepin-3-yl]oxy]pentoxy]-6-hydroxy-2-methoxy-8-methyl-11-oxo-6a,7-dihydro-6H-pyrrolo[2,1-c][1,4]benzodiazepine-5-carboxylate), temsirolimus (same as [(1R,2R,4S)-4-[(2R)-2-[(1R,9S,12S,15R,16E,18R,19R,21R,23 S,24E,26E,28E,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl] 3-hydroxy-2-(hydroxymethyl)-2-methylpropanoate), tivantinib, tivozanib (same as 1-{2-chloro-4-[(6,7-dimethoxyquinolin-4-yl)oxy]phenyl}-3-(5-methylisoxazol-3-yl)urea), vatalanib, veliparib, or vinblastine; or variants of aforementioned structures derived from these by modification(s) of said structure(s); or

e) (H)ₙR¹ and (H)ₒR² are independently compound(s) active against a kidney cancer disease; or

f) (H)ₙR¹ is a heterocyclic structure(s) connected to X at one of heterocyclic nitrogen atom(s) present within the structure (H)ₙR¹; wherein said nitrogen atom becomes a nitrogen atom with a single positive charge, such as imidazolium, pyrazolium, pyridinium, or indazolium group; and

when an optional group R¹ is absent, then the fragment R¹X is replaced with R^{11a}, wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein

when an optional group R² is absent, then the fragment R²Z is replaced with R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein

integers n and o are independently selected from 0, 1, 2, 3, 4, 5, 6, and 7, such that [n+o]≥1; and

A¹ through A¹¹ are optional amino acid residues independently selected from unsubstituted or substituted at any N atom alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and

integers a through m are independently selected from 0, 1, and 2, and wherein

[m+l]≥1, wherein the symbol “l” in [m+l] and at the group [R¹—X]ₗ represents a letter “l”; and wherein

when any of integers a through k is 0, then any of the two groups adjacent to a respective absent group (according to the integer 0 at said absent group) are connected to each other directly; and wherein

when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, or heterocyclyl; or the group A⁸ is directly connected to the group Y; and

each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,

(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁵), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),

C(═O)N[(CR⁵R⁶)ₚ(CR⁷R⁸)ᵣN(C₁₋₆alkyl)C(═O)A¹⁴](CR⁹R¹⁰)ₛOC(═O),

C(═O)N[(CR⁵R⁶)ₚ(CR⁷R⁸)ᵣOC(═O)A¹⁴](CR⁹R¹⁰)ₛC(═O),

C(═O)N[(CR⁵R⁶)ₚ(CR⁷R⁸)ᵣOC(═O)A¹⁴](CR⁹R¹⁰)ₛN(C═O),

C(═O)N[(CR⁵R⁶)ₚ(CR⁷R⁸)ᵣOC(═O)A¹⁴](CR⁹R¹⁰)ₛO(C═O),

C(═O)N[(CR⁵R⁶)ₚ(CR⁷R⁸)ᵣNHC(═O)A¹⁴A¹⁵](CR⁹R¹⁰)ₛC(═O),

C(═O)N[(CR⁵R⁶)ₚ(CR⁷R⁸)ᵣNHC(═O)A¹⁴A¹⁵](CR⁹R¹⁰)ₛN(C═O),

C(═O)N[(CR⁵R⁶)ₚ(CR⁷R⁸)ᵣNHC(═O)A¹⁴A¹⁵](CR⁹R¹⁰)ₛO(C═O),

C(═O)N[(CR⁵R⁶)ₚ(CR⁷R⁸)ᵣOC(═O)A¹⁴A¹⁵](CR⁹R¹⁰)ₛC(═O),

C(═O)N[(CR⁵R⁶)ₚ(CR⁷R⁸)ᵣOC(═O)A¹⁴A¹⁵](CR⁹R¹⁰)ₛN(C═O),

C(═O)N[(CR⁵R⁶)ₚ(CR⁷R⁸)ᵣOC(═O)A¹⁴A¹⁵](CR⁹R¹⁰)ₛO(C═O),

C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),

C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),

C(═O)N[CH₂CH₂NHC(═O)CH₂CH₂CH(NH₂)COOH](CR⁵R⁶)ₚCR⁷R⁸)ᵣ OC(═O) (CR⁹R¹⁰)ₛC(═O),

C(═O)N[CH₂CH₂NHC(═O)CH₂CH₂CH(NH₂)COOH](CR⁵R⁶)ₚC(═O)O(CR⁷R⁸)ᵣ (CR⁹R¹⁰)ₛC(═O),

C(═O)N[CH₂CH₂NHC(═O)CH₂CH₂CH(NH₂)COOH](CR⁵R⁶)ₚOC(═O)(CR⁷R⁸)ᵣ (CR⁹R¹⁰)ₛC(═O),

C(═O)N[CH₂CH₂NHC(═O)CH₂CH₂CH(NH₂)COOH]CH₂CH₂C(═O)OCH₂CH₂C(═O),

C(═O)N[CH₂CH₂NHC(═O)CH₂CH₂CH(NH₂)COOH]CH₂CH₂OC(═O)CH₂CH₂C(═O),

C(═O)N[CH₂CH₂NHC(═O)CH₂CH₂CH(NH₂)COOH]CH₂CMe₂C(═O)OCH₂CH₂C(═ O),

C(═O)N[CH₂CH₂NHC(═O)CH₂CH₂CH(NH₂)COOH]CH₂CH₂OC(═O)CH(Me)-CH₂C(═O),

C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),

(S)—C(═O)N[CH₂CH₂NHC(═O)CH₂CH₂CH(NH(C═O)R⁷)COOH]CH₂CH₂C(═O),

(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),

(S)—C(═O)N[CH₂CH₂NHC(═O)CHNH(CH₂CH₂CH₂CH₂NH₂)]CH₂CH₂C(═O),

(R)—C(═O)N[CH₂CH₂NHC(═O)CHNH(CH₂CH₂CH₂CH₂NH₂)]CH₂CH₂C(═O),

(S)—C(═O)N[CH₂CH₂NHC(═O)CHNH(CH₂CH₂NH₂)]CH₂CH₂C(═O),

(R)—C(═O)N[CH₂CH₂NHC(═O)CHNH(CH₂CH₂NH₂)]CH₂CH₂C(═O),

or any variant of above groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein

R⁴, R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; and wherein

R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein

any two of R⁴ through R¹⁰, together with the atom(s) to which they are attached form a 4 to 7-member saturated or unsaturated heterocycle containing at least one 0 atom, or containing one 0 atom and an additional heteroatom independently selected from N and S and wherein remaining atoms are carbon; or wherein

any two of R⁴ through R¹⁰, together with the carbon atom(s) to which they are attached form a 4 to 7-member saturated or unsaturated C₃₋₆cycloalkylene; or any of i) R⁴ and R⁵, ii) R⁶ and R⁷, iii) R⁴ and R⁶, and iv) R⁹ and R¹⁰, together with the atom to which they are attached form a saturated or unsaturated C₃₋₆cycloalkylene; or wherein

any two of R⁴ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein

R⁶ and R⁸ together with the atom to which they are attached form a 4 to 6-member saturated heterocycle containing at least one 0 atom wherein the heterocycle optionally comprises an additional heteroatom selected from N, O, and S, and wherein the remaining atoms are carbon; or the resulting ring comprises 1,3-dioxol-2-one heterocycle; and wherein

integers p, r, and s are independently selected from 0, 1, and 2; and wherein when fragments (CR⁴R⁵)ₚ(CR⁶R⁷)ᵣ(CR⁹R¹⁰)s or (OCR⁴R⁵)ₚ(CR⁶R⁷)ᵣ(CR⁹R¹⁰)s are present, then [p+r+s]≥1; and wherein

when fragments (CR⁴R⁵)ₚ(CR⁶R⁷)ᵣ or (OCR⁴R⁵)ₚ(CR⁶R⁷)ᵣ are present, then [p+r]≥1; and wherein

when fragments (CR⁷R⁸)ᵣ(CR⁹R¹⁰)s or (OCR⁷R⁸)ᵣ(CR⁹R¹⁰)s are present, then [r+s]≥1: or

alternatively, each optional divalent group X is independently comprised of the following structures, optionally connected to one to two amino acid residue(s) A¹² or A¹³, with the following structures:

(C₁₋₁₂alkylene)OC(═O), OC(═O) (C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ᵣN(R⁵)C(═O) C(═O)CR⁵═CR⁷—(CR⁹R¹⁰)ₚC(═O),
C(═O)OCR⁵═CR⁷—(CR⁹R¹⁰)ₚC(═O), C(═O)N(R⁵)CR⁵═CR⁷—(CR⁹R¹⁰)ᵣC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₚC(═O),
C(═O)N(R⁵)CR⁵═CR⁷—(CR⁹R¹⁰)ᵣC(═O),
C(═O)CR⁵═CR⁷—(CR⁹R¹⁰)ₚOC(═O),
C(═O)CR⁵═CR⁷—(CR⁹R¹⁰)ᵣN(R⁵)C(═O), or any variant of the above X groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein

when an optional group X is absent, then group R¹ is directly connected to one of groups A⁸, A⁹, A¹⁰, or A¹¹; or

additionally, each optional divalent group X independently incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂-)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂-)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₄alkyl)(CH₂)ₚO(CH₂)ᵣO(CH₂)ₛN(C₁₋₁₄alkyl)C(═O), or similar linear groups;

optional divalent groups Y and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,

(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R⁵), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),

C(═O)N[(CR⁵R⁶)ₚ(CR⁷R⁸)ᵣN(C₁₋₆alkyl)C(═O)A¹⁴](CR⁹R¹⁰)ₛOC(═O),

C(═O)N[(CR⁵R⁶)ₚ(CR⁷R⁸)ᵣOC(═O)A¹⁴](CR⁹R¹⁰)ₛC(═O),

C(═O)N[(CR⁵R⁶)ₚ(CR⁷R⁸)ᵣOC(═O)A¹⁴](CR⁹R¹⁰)ₛN(C═O),

C(═O)N[(CR⁵R⁶)ₚ(CR⁷R⁸)ᵣOC(═O)A¹⁴](CR⁹R¹⁰)ₛO(C═O),

C(═O)N[(CR⁵R⁶)ₚ(CR⁷R⁸)ᵣNHC(═O)A¹⁴A¹⁵](CR⁹R¹⁰)ₛC(═O),

C(═O)N[(CR⁵R⁶)ₚ(CR⁷R⁸)ᵣNHC(═O)A¹⁴A¹⁵](CR⁹R¹⁰)ₛN(C═O),

C(═O)N[(CR⁵R⁶)ₚ(CR⁷R⁸)ᵣNHC(═O)A¹⁴A¹⁵](CR⁹R¹⁰)ₛO(C═O),

C(═O)N[(CR⁵R⁶)ₚ(CR⁷R⁸)ᵣOC(═O)A¹⁴A¹⁵](CR⁹R¹⁰)ₛC(═O),

C(═O)N[(CR⁵R⁶)ₚ(CR⁷R⁸)ᵣOC(═O)A¹⁴A¹⁵](CR⁹R¹⁰)ₛN(C═O),

C(═O)N[(CR⁵R⁶)ₚ(CR⁷R⁸)ᵣOC(═O)A¹⁴A¹⁵](CR⁹R¹⁰)ₛO(C═O),

C(═O)N[(CR⁵R⁶)ₚ(CR⁷R⁸)ᵣNHC(═O)(CR⁹R¹⁰)ₛNCH(NH₂)COOH]CH₂CH₂C(═O), C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),

C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),

C(═O)N[CH₂CH₂NHC(═O)CH₂CH₂CH(NH₂)COOH](CR⁵R⁶)ₚCR⁷R⁸)ᵣ OC(═O) (CR⁹R¹⁰)ₛC(═O),

C(═O)N[CH₂CH₂NHC(═O)CH₂CH₂CH(NH₂)COOH](CR⁵R⁶)ₚC(═O)O(CR⁷R⁸)ᵣ (CR⁹R¹⁰)ₛC(═O),

C(═O)N[CH₂CH₂NHC(═O)CH₂CH₂CH(NH₂)COOH](CR⁵R⁶)ₚ OC(═O)(CR⁷R⁸)ᵣ (CR⁹R¹⁰)ₛC(═O),

C(═O)N[CH₂CH₂NHC(═O)CH₂CH₂CH(NH₂)COOH]CH₂CH₂C(═O)OCH₂CH₂C(═O),

C(═O)N[CH₂CH₂NHC(═O)CH₂CH₂CH(NH₂)COOH]CH₂CH₂OC(═O)CH₂CH₂C(═O),

C(═O)N[CH₂CH₂NHC(═O)CH₂CH₂CH(NH₂)COOH]CH₂CMe₂C(═O)OCH₂CH₂C(═ O),

C(═O)N[CH₂CH₂NHC(═O)CH₂CH₂CH(NH₂)COOH]CH₂CH₂OC(═O)CH(Me)-CH₂C(═O),

C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),

(S)—C(═O)N[CH₂CH₂NHC(═O)CH₂CH₂CH(NH(C═O)R⁷)COOH]CH₂CH₂C(═O),

(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),

(S)—C(═O)N[CH₂CH₂NHC(═O)CHNH(CH₂CH₂CH₂CH₂NH₂)]CH₂CH₂C(═O),

(R)—C(═O)N[CH₂CH₂NHC(═O)CHNH(CH₂CH₂CH₂CH₂NH₂)]CH₂CH₂C(═O),

(S)—C(═O)N[CH₂CH₂NHC(═O)CHNH(CH₂CH₂NH₂)]CH₂CH₂C(═O),

(R)—C(═O)N[CH₂CH₂NHC(═O)CHNH(CH₂CH₂NH₂)]CH₂CH₂C(═O), or

any variant of above groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein R⁵-R¹⁰ are as defined above; or

alternatively, the optional group Z is comprised of the following structures, optionally connected to one to two amino acid residue(s) A¹² or A¹³, with the following structures:

(C₁₋₁₂alkylene)OC(═O), OC(═O) (C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₚC(═O),
C(═O)OCR⁵═CR⁷—(CR⁹R¹⁰)ₚC(═O), C(═O)N(R⁵)CR⁵═CR⁷—(CR⁹R¹⁰)ᵣC(═O),
C(═O)CR⁵═CR⁷—(CR⁹R¹⁰)ₚOC(═O), C(═O)CR⁵═CR⁷—(CR⁹R¹⁰)ᵣN(R⁵)C(═O); or any variant of the above Z groups formed by repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² and A¹³ are incorporated at the left side of above groups to comprise the group Z, then residues A¹² or A¹³ are interconnected with a peptide bond A¹²-A¹³; and

when an optional group Z is absent, then the group R² is directly connected to one of groups Y, A¹, A², A³, A⁴, A⁵, A⁶, A⁷ or A⁸.

In another aspect, provided herein is a compound of formula I-P:

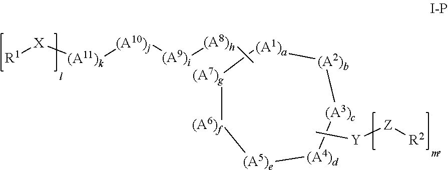

or a pharmaceutically acceptable salt, solvate, or hydrate thereof wherein:

R¹ and R² are optional groups, with at least one of the groups R¹ and R² being present in the formula I-P; and

R¹ and R² are independently selected from alkyl, aryl, biaryl, heteroaryl, heteroarylaryl, and arylheteroaryl; or

R¹ and R² are structures independently derived by subtracting a single or multiple H atom(s) from respective parent (precursor) structure(s) (H)ₙR¹ and (H)ₒR² at any one of optional H-containing group(s) independently selected from NH, OH, SH, C(═O)OH, CONH, SO₂NH, and S(═O)NH present in said structures (H)ₙR¹ and (H)ₒR²; and wherein

the structures (H)ₙR¹ and (H)ₒR² are independently structures of compounds possessing biological or therapeutic activity; or

the structures (H)ₙR¹ and (H)ₒR² are structures of cytotoxic, antibody, or immunomodulating compounds possessing an activity or capable of inducing activity against cancer cells, including compounds with activity against renal cancer cells; or

the structures (H)ₙR¹ and (H)ₙR² are structures of mono- or multi-valent antibody(ies) with activity against cancer cells; or

the structures (H)ₙR¹ and (H)ₒR² are independently afatinib (same as (E)-N-[4-(3-chloro-4-fluoroanilino)-7-[(3S)-oxolan-3-yl]oxyquinazolin-6-yl]-4-(dimethylamino)but-2-enamide), ARS-1630, axitinib (same as N-methyl-2-[[3-[(E)-2-pyridin-2-ylethenyl]-1H-indazol-6-

yl]sulfanyl]benzamide), BGB-324, BLU-554, brivanib (same as (S)—(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate), (R)-1-((4-((4-fluoro-2-methyl-1H-indol-5-yl)oxy)-5-methylpyrrolo[2,1-f][1,2,4]triazin-6-yl)oxy)propan-2-ol, cabozantinib, cediranib, ceritinib, ciforadenant, derazantinib, dovitinib (same as 4-amino-5-fluoro-3-(6-(4-methylpiperazin-1-yl)-1H-benzo[d]imidazol-2-yl)quinolin-2(1H)-one), E-7046, emtansine, englerin (same as (1R,3aR,4S,5R,7R,8S,8aR)-5-(glycoloyloxy)-7-isopropyl-1,4-dimethyldecahydro-4,7-epoxyazulen-8-yl (2E)-3-phenylacrylate), foretinib, lenvatinib (same as 4-[3-chloro-4-(cyclopropylcarbamoylamino)phenoxy]-7-

methoxyquinoline-6-carboxamide), monomethyl auristatin E (same as (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide), irinotecan, maytansinoid, neratinib, nilotinib, nintedanib, ozogamicin, paclitaxel, pazopanib (same as 5-[[4-[(2,3-dimethylindazol-6-yl)-methylamino]pyrimidin-2-yl]amino]-2-methylbenzenesulfonamide), regorafenib, sacituzumab, selpercatinib, semaxanib (same as (Z)-3-((3,5-dimethyl-1H-pyrrol-2-yl)methylene)indolin-2-one), sorafenib (same as 4-[4-[[4-chloro-3-(trifluoromethyl)phenyl]carbamoylamino]phenoxy]-N-methylpyridine-2-carboxamide), sunitinib (same as (Z)—N-(2-(diethylamino)ethyl)-5-((5-fluoro-2-oxoindolin-3-ylidene)methyl)-2,4-dimethyl-1H-pyrrole-3-carboxamide), SN38 (same as 7-ethyl-10-hydroxy-camptothecin), sorafenib (same as 4-[4-[[4-chloro-3-(trifluoromethyl)phenyl]carbamoylamino], phenoxy]-N-methyl-pyridine-2-carboxamide), sunitinib (same as (Z)—N-(2-(diethylamino)ethyl)-5-((5-fluoro-2-oxoindolin-3-ylidene)methyl)-2,4-dimethyl-1H-pyrrole-3-carboxamide), trastuzumab, tesirine (same as [4-[[(2S)-2-[[(2S)-2-[3-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]propanoylamino]-3-methylbutanoyl]amino]propanoyl]amino]phenyl]methyl (6S,6aS)-3-[5-[[(6aS)-2-methoxy-8-methyl-11-oxo-6a,7-dihydropyrrolo[2,1-c][1,4]benzodiazepin-3-yl]oxy]pentoxy]-6-hydroxy-2-methoxy-8-methyl-11-oxo-6a,7-dihydro-6H-pyrrolo[2,1-c][1,4]benzodiazepine-5-carboxylate), temsirolimus (same as [(1R,2R,4S)-4-[(2R)-2-[(1R,9S,12S,15R,16E,18R,19R,21R,23 S,24E,26E,28E,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl] 3-hydroxy-2-(hydroxymethyl)-2-methylpropanoate), tivantinib, tivozanib, vatalanib, veliparib, vinblastine; or variants of aforementioned structures derived from these by an obvious in the synthetic art chemical modification(s) of said structure(s); or

the structures (H)ₙR¹ and (H)ₒR² are independently structures of compounds active against a kidney disease; and wherein

when an optional group R¹ is absent, then the fragment R¹X is replaced for group R^{11a}, wherein Ria is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein

when an optional group R² is absent, then the fragment R²Z is replaced for group R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), aryl N(C₁. 12alkyl)C(═O), arylSO₂, arylNHSO₂, heteroarylC(═O), heteroarylOC(═O), heteroarylNHC(═O), heteroarylN(C₁₋₁₂alkyl)C(═O), heteroarylSO₂, and heteroarylNHSO₂; or wherein

(H)ₙR¹ is heterocyclic structure(s) connected to X at one of heterocyclic nitrogen atom(s) present within the structure (H)ₒR¹; wherein said nitrogen atom becomes a nitrogen atom with a single positive charge, imidazolium, pyrazolium, pyridinium, or indazolium group; and

integers n and o are independently selected from 0, 1, 2, 3, 4, 5, 6, and 7, such that [n+o]≥1; and

A¹ through A¹¹ are optional amino acid residues independently selected from substituted or unsubstituted at any N atom alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid; and

integers a through m are independently selected from 0, 1, and 2, and wherein

[m+i]≥1, wherein the symbol “l” in [m+i] represents a letter “l”; and wherein

when any of integers a through k is 0, then any of the two groups adjacent to a respective absent group (according to the integer 0 at said absent group) are connected to each other directly; and wherein

when both integers selected from a through k at adjacent groups A¹-A², A³-A⁴, A⁵-A⁶, or A¹-A⁷ are 0, then respective pairs of adjacent groups A¹-A², A³-A⁴, A⁵-A⁶, or A¹-A⁷ are absent, to result in acyclic structure of formula I-P; wherein one of remaining groups selected from A¹-A⁷ is connected to either group A⁸ or group Y, and the last amino acid residue of resulted peptide sequence (which is not connected to either group A⁸ or group Y) terminates with groups COOH, CH₂OH, or C(═O)NR³R⁴, wherein R³ and R⁴ are independently selected from H, alkyl, aryl, heteroaryl, and heterocyclyl; or wherein

when the integers a through g are all 0, then the groups A¹-A⁷ are absent, and the group A⁸ terminates with either COOH, CH₂OH, or C(═O)NR³R⁴; or the group A⁸ is directly connected to the group Y; and

optional divalent groups X, Y, and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,

(C₁₋₁₂alkylene)C(═O)O, OC(═O) (C₁₋₁₂alkylene),
(C₁₋₁₂alkylene)OC(═O), C(═O)O(C₁₋₁₂alkylene),
(C₁₋₁₂alkylene)C(═O)N(R⁵), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)SN(R⁵)C(═O), and

any variant of above groups formed by straightforward repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein

R⁴, R⁶, R⁷, R⁹ and R¹⁰ are independently H, NH₂, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; and wherein

R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl; or wherein

any two of R⁴ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein

any two of R⁴ through R¹⁰, together with the carbon atom(s) to which they are attached form a 4 to 7-member saturated or unsaturated C₃₋₆cycloalkylene; or any of i) R⁴ and R⁵, ii) R⁶ and R⁷, iii) R⁴ and R⁶, and iv) R⁹ and R¹⁰, together with the atom to which they are attached form a C₃₋₆cycloalkylene; or wherein

any two of R⁴ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein

integers p, r, and s are independently selected from 0, 1, and 2; and wherein when fragments (CR⁴R⁵)ₚ(CR⁶R⁷)ᵣ(CR⁹R¹⁰)s or (OCR⁴R⁵)ₚ(CR⁶R⁷)ᵣ(CR⁹R¹⁰)ₛ are present, then [p+r+s]≥1; and wherein

when fragments (CR⁴R⁵)ₚ(CR⁶R⁷)ᵣ or (OCR⁴R⁵)ₚ(CR⁶R⁷)ᵣ are present, then [p+r]≥1; and wherein

when fragments (CR⁷R⁸)ᵣ(CR⁹R¹⁰)ₛ or (OCR⁷R⁸)ᵣ(CR⁹R¹⁰)ₛ are present, then [r+s]≥1; or

X, Y, and Z are independently selected from one to four amino acid residue(s) A¹², A¹³, A¹⁴, and A¹⁵ connected to each other with peptide bonds; wherein

A¹², A¹³, A¹⁴, or A¹⁵ are independently substituted or unsubstituted at any N atom alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid, or 5-amino-4-arylpentanoic acid, or similar natural or unnatural amino acid residues; or

X is a group comprised of the following structures, additionally connected to one to two amino acid residue(s) A¹² or A¹³, at the right side of the following structures:

(C₁₋₁₂alkylene)OC(═O), OC(═O) (C₁₋₁₂alkylene),
(C₁₋₁₂alkylene)OC(═O), C(═O)O(C₁₋₁₂alkylene),
(C₁₋₁₂alkylene)C(═O), N(R⁵)C(═O)(C₁₋₁₂alkylene),
(C₁₋₁₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O); or any variant of these groups formed by straightforward repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² or A¹³ are incorporated at the right side of above groups to comprise the group X, then residues A¹² or A¹³ are interconnected with a peptide bond A¹²-A¹³; and wherein

when an optional group X is absent, then group R¹ is directly connected to one of groups A⁸, A⁹, A¹⁰, or A¹¹; or

Z is a group comprised of the following structures, additionally connected to one to two amino acid residue(s) A¹² or A¹³, at the left side of the following structures:

(C₁₋₂alkylene)OC(═O), OC(═O) (C₁₋₂alkylene),
(C₁₋₂alkylene)OC(═O), C(═O)O(C₁₋₂alkylene),
(C₁₋₂alkylene)C(═O), N(R⁵)C(═O)(C₁₋₂alkylene),
(C₁₋₂alkylene)N(R⁵)C(═O), C(═O) N(R⁵)(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)P,
C(═O)O(CR⁵R⁶)ₚ(CR⁷R⁸)ᵣ(CR⁹R¹⁰)ₛC(═O), C(═O)N(R⁵)(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O), C(═O)(CR⁵R⁶)ₚ(CR⁷R⁸)ᵣ(CR⁹R¹⁰)ₛN(R⁵)C(═O)
C(═O)CR⁵═CR⁷—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O),
C(═O)N(R⁵)SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R⁵)SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R⁵)C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R⁵)(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R⁵)C(═O)
C(═O)CR⁵═CR⁷—S—S—(CR⁹R¹⁰)ₛC(═O),
C(═O)OCR⁵═CR⁷—(CR⁹R¹⁰)ₛC(═O), C(═O)N(R⁵)CR⁵═CR⁷—(CR⁹R¹⁰)ₛC(═O),
C(═O)CR⁵═CR⁷—(CR⁹R¹⁰)ₛOC(═O), C(═O)CR⁵═CR⁷—(CR⁹R¹⁰)ₛN(R⁵)C(═O); or any variant of these groups formed by straightforward repositioning(s), addition(s), or deletion(s) of the fragments C(═O), OC(═O), N(R⁵)C(═O), P(═O)(OCR⁵R⁶)CF₂, P(═O)(OH)CF₂, or C(═O)N(R⁵)SO₂ therein; and wherein when both amino acid residues A¹² or A¹³ are incorporated at the left side of above groups to comprise the group Z, then residues A¹² or A¹³ are interconnected with a peptide bond A¹²-A¹³; and

when an optional group Z is absent, then the group R² is directly connected to one of groups Y, A¹, A², A³, A⁴, A⁵, A⁶, A⁷ or A⁸.

In one aspect, provided is a compound of formula I-P or formula I, wherein the integers a through g are all equal to 1; and wherein A¹ is Thr or Ser; A², A³ A⁶, and A⁷ are independently selected from Dab, Dap, Ser, or Thr; A⁴ is Leu or Ile; and A⁵ is Phe, D-Phe, Bip, D-Bip, Val, and D-Val.

In additional aspect, provided is a compound of formula I-P or formula I where group X, either at its left or right side therein, incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂-)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂-)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂-)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂-)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups.

In another aspect, provided is a compound of formula I-P or formula I, wherein a cyclic peptide structure comprised of optional amino acid residues A¹ through A⁷ is a cyclic peptide structure identical to that present in polymyxin B, polymyxin E, or octapeptin, or similar cyclic peptide structures. In another embodiment, provided is a compound of formula I-P above, wherein a cyclic peptide structure comprised of optional amino acid residues A¹ through A⁷ is a cyclic peptide structure identical to that present in polymyxin B, polymyxin E, or octapeptin, or similar structures, including cyclic peptide structures. In another embodiment, provided is a compound of formula I, wherein a cyclic peptide structure comprised of optional amino acid residues A¹ through A⁷ is a cyclic peptide structure identical to that present in polymyxin A, polymyxin B, polymyxin B nonapeptide (H-Thr-Dab-cyclo[Dab-Dab-D-Phe-Leu-Dab-Dab-Thr]), polymyxin B heptapeptide (H-cyclo[Dab-Dab-D-Phe-Leu-Dab-Dab-Thr]), polymyxin E, or octapeptin, or similar structures, including cyclic peptide structures.

In additional aspect, provided is a compound of any one of formulas I-P, II-P, III-P, and I-V, of any one of embodiments 2-8, or of any embodiments as provided herein that exhibits a therapeutic effect after administration into a mammal by releasing a bioactive or cytotoxic molecules (H)ₙR¹ and (H)ₒR².

In another aspect, provided is a compound of any one of formulas I-P, II-P, III-P, and I-V, of any one of embodiments 2-8, or of any embodiments as provided herein possessing anticancer activity against cancerous cells, as determined by inhibition or slowing of cancer(s) cells growth using in vitro cytotoxicity test(s) or assay(s), or by testing of said compounds in animal models for cancer(s).

In additional aspect, aforementioned cancer is a renal cancer or a kidney cancer.

In another aspect, provided is a method for the treatment of a kidney cancer disease in a mammal comprising administering to the mammal a therapeutically effective amount of a compound of any one of formulas I-P-P, II-P, III-P, and I-V, of any one of embodiments 2-8, or of any embodiments as provided herein.

In another aspect, provided is a compound of any one of formulas I-P, II-P, III-P, and I-V, of any one of embodiments 2-8, or of any embodiments as provided herein and with a reduced cytotoxicity against non-cancerous mammalian cell(s), when compared to a related cytotoxic structure (compound, e.g. (H)ₙR¹ and/or (H)ₒR²) incorporated into said compound(s), as determined by in vitro cytotoxicity test(s) or assay(s).

In another aspect, provided is a compound of any one of formula I-P, II-P, III-P, and I-V, of any one of embodiments 2-8, or of any embodiments as provided herein possessing an increased in vivo efficacy against cancer(s), when compared to a related (parent) cytotoxic structure (compound, e.g. (H)ₙR¹ and/or (H)ₒR²) incorporated into said compound(s), as determined by in vivo test(s) in animal model(s) of cancer(s), wherein said compound and a related cytotoxic structure (compound, e.g. (H)ₙR¹ and/or (H)ₒR²) are dosed to an animal at identical molar dose of a common cytotoxic structure within the test and the comparator compounds.

In yet another aspect, provided is a compound of any one of formula I-P, II-P, III-P, and I-V, of any one of embodiments 2-8, or of any embodiments as provided herein possessing at least 2-fold increase in vivo efficacy against cancer(s), when compared to a related cytotoxic structure (compound) incorporated into said compound(s).

In additional aspect is provided a pharmaceutical composition comprising a compound of any one of formula I-P, II-P, III-P, and I-V, of any one of embodiments 2-8, or of any embodiments as provided herein, or a pharmaceutically acceptable salt, prodrug, solvate, or hydrate thereof, and a pharmaceutically acceptable carrier, excipient or diluent.

In an another aspect is provided a method for treating a kidney cancer in humans or other warm-blooded animals by administering to the subject in need a therapeutically effective amount of a compound of any one of formulas I-P, II-P, III-P, and I-V, of any one of embodiments 2-8, or of any embodiments as provided herein or a pharmaceutically acceptable salt, prodrug, solvate, or hydrate thereof

The compound of any one of formulas I-P, II-P, III-P, and I-V, of any one of embodiments 2-8, or of any embodiments as provided herein may be administered, for example, orally, parenterally, transdermally, topically, rectally, or intranasally, or via an intra-tumoral administration.

In yet another aspect is provided novel intermediates and processes for preparing compounds of any one of formulas I-P, II-P, III-P, and I-V, of any one of embodiments 2-8, or of any embodiments as provided herein.

### DETAILED DESCRIPTION OF THE INVENTION

Unless otherwise stated, the following terms used in the specification and Claims have the meanings given below.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₁₄ alkyl refers to alkyl of one to fourteen carbon atoms, inclusive.

The term alkyl refers to both straight and branched saturated hydrocarbon groups. Reference to an individual radical such as “propyl” embraces only the straight chain radical, and a branched chain isomer such as “isopropyl” being specifically referred to. Unless specified otherwise “alkyl” contains 1-12 carbon atoms. In addition to any group specifically recited in any of the embodiments or claims, the alkyl group is optionally substituted with one, two, three, or four substituents selected from the group consisting of halo, hydroxy, cyano, C₁₋₂ alkyl, C₃₋₇cycloalkyl, aryl, biaryl, heterocyclic, or heteroaryl (Het) group. In some embodiments, alkyl includes, but is not limited to, difluoromethyl, 2-fluoroethyl, trifluoroethyl, (adamantane-1-yl)methyl, 3-(cyclohexyl)propyl, 4-propylcyclohexyl, —CH═CH-aryl, —CH═CH—Het¹, —CH₂-phenyl, biphenylmethyl, and the like. In some embodiments, alkyl is unsubstituted. Alkyl groups distinguished as “alkyl” and “alkyl¹” or “alkyl²” refer to independently selected alkyl groups that may be different from each other, or independently equal to each other. If the term “alkyl” is used more than once in the same group, then each “alkyl” is independent of another “alkyl”, at each appearance.

The term “Alk” refers to alkyl, as defined herein.

The term “alkylene” refers to a divalent alkyl group. Unless specified otherwise linear “alkylene” contains 1-12 carbon atoms. The alkylene group is optionally substituted as described for alkyl. In some embodiments, alkylene is unsubstituted. Alkylene groups distinguished as “alkylene” and “alkylene¹” or “alkylene²” refer to independently selected alkylene groups that may be different from each other, or independently equal to each other.

The term “alkenyl” refers to both straight and branched hydrocarbon groups containing at least one double bond, and in some embodiments 1, 2, or 3 double bonds. Unless specified otherwise “alkenyl” contains 2-12 carbon atoms. In addition to any group specifically recited in any of the embodiments or claims, the alkenyl is optionally substituted with one, two, or three substituents selected from the group consisting of halo, C₁₋₁₂ alkyl, C₃₋₇cycloalkyl, aryl, biaryl, Het¹, and Het². In some embodiments, alkenyl includes, but is not limited to, difluoromethyl, 2-fluoroethyl, trifluoroethyl, (adamantane-1-yl)methyl, 3-(cyclohexyl)propyl, 4-propylcyclohexyl, —CH═CH-aryl, —CH═CH—Het¹, —CH₂-phenyl, biphenylmethyl, and the like. In some embodiments, alkenyl is unsubstituted.

The term “alkenylene” refers to a divalent alkenyl group. Unless specified otherwise “alkenylene” contains 2-12 carbon atoms. The alkenylene group is optionally substituted as described for alkenyl. In some embodiments, the alkenylene group is unsubstituted.

The term “cycloalkyl” or “carbocycle” means a cyclic saturated, monovalent, monocyclic or bicyclic, saturated or unsaturated hydrocarbon group of three to 18 (in some embodiments, three to six) carbon atoms. In some embodiments, cycloalkyl includes but is not limited to cyclopropyl, cyclohexyl, cyclododecanoyl, and the like. In addition to any group specifically recited in any of the embodiments or claims, the cycloalkyl group is optionally substituted with one, two, or three substituents selected from the group consisting of halo, C₁₋₁₂ alkyl, C₃₋₇cycloalkyl, aryl, and Het or heteroaryl. In some embodiments, cycloalkyl is unsubstituted.

The term “cycloalkylene” means a divalent cycloalkyl group or divalent carbocycle group. In addition to any group specifically recited in any of the embodiments or claims, the cycloalkylene group is optionally substituted as described for cycloalkyl. In some embodiments, the cycloalkylene is unsubstituted. In some or any embodiments, the C₃₋₆cycloalkylene group formed by any two of R⁴ through R¹⁰ is optionally substituted with one or two groups independently selected from C₁₋₆alkyl and aryl.

The term “heteroalkyl” means an alkyl or cycloalkyl group, as defined above, having a substituent containing a heteroatom selected from N, O, and S(O)n, where n is an integer from 0 to 2, where in some embodiments the substituent includes, hydroxy (OH), C₁₋₄alkoxy, amino, thio (—SH), and the like. Said heteroatom may be incorporated in any part of the heteroalkyl group [e.g., heteroalkyl can be C₁₋₄alkylC(═O)O C₃₋₆cycloalkylNH₂], or contain a heterocyclic substituent [e.g., heteroalkyl can be 2-(4-morpholino)ethyl]. In some embodiments, substituents include —NRₐR_{b}, —ORₐ, and —S(O)ₙR_{c}, wherein each Rₐ is independently hydrogen, C₁₋₄alkyl, C₃₋₆cycloalkyl, optionally substituted aryl, optionally substituted heterocyclic, or —C(O)R (where R is C₁₋₄alkyl); each R_{b} is independently hydrogen, C₁₋₁₄alkyl, —SO₂R (where R is C₁₋₄alkyl or C₁₋₄hydroxyalkyl), —SO₂NRR′ (where R and R′ are independently of each other hydrogen or C₁₋₄alkyl), or —CONR′R″ (where R′ and R″ are independently of each other hydrogen or C₁₋₄alkyl); n is an integer from 0 to 2; and each R_{c} is independently hydrogen, C₁₋₄alkyl, C₃₋₆cycloalkyl, optionally substituted aryl, or NRₐR_{b} where Rₐ and R_{b} are as defined above. In some embodiments, heteroalkyl includes, but is not limited to 2-methoxyethyl (—CH₂CH₂OCH₃), 2-hydroxyethyl (—CH₂CH₂OH), hydroxymethyl (—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to substituted or unsubstituted phenyl, biphenyl, triphenyl, or naphthyl. In addition to any group specifically recited in any of the embodiments or claims, the aryl is optionally substituted with 1 to 3 substituents independently selected from halo, —C₁₋₁₂alkyl (unsubstituted or substituted, in one embodiment with 1, 2, or 3 halo), aryl, —OH, —OC₁₋₁₂alkyl, —S(O)ₙC₁₋₄alkyl (wherein n is 0, 1, or 2), —C₁₋₄alkylNH₂, —NHC₁₋₄alkyl, —C(═O)H, C(═O)OR^{a}, OC(═O)R^{a}, OC(═O)NR^{a}R^{c}, OC(═O)heteroaryl, OC(═O)(heterocyclic ring) and —C═N—OR_{d} wherein R_{d} is hydrogen or —C₁₋₄alkyl. Two adjacent substituents in the aryl group may be connected to form a C₄₋₇cycloalkyl or 4- to 7-member heterocyclic group fused to said aryl group. Aryl groups distinguished as “aryl” and “aryl¹” or “aryl²” refer to independently selected aryl groups that may be different from each other, or independently equal to each other. If the term “aryl” is used more than once in the same group, then each “aryl” is independent of another “aryl,” at each appearance.

The term “arylene” refers to a divalent aryl group, as defined herein.

The term “arylalkyl” refers to an alkyl group substituted with an aryl group, each as defined herein, including where the aryl and alkyl are optionally substituted as described in their respective definitions.

The term “arylheteroaryl” refers to an aryl group substituted with a heteroaryl group, each as defined herein, including where the aryl and heteroaryl are optionally substituted as described in their respective definitions.

The term “(heteroaryl)arylene” refers to a divalent aryl group, as defined herein, substituted with a heteroaryl group.

The term “heteroarylaryl” refers to a heteroaryl group substituted with an aryl group, each as defined herein, including where the aryl and heteroaryl are optionally substituted as described in their respective definitions.

The term “(aryl)heteroarylene” refers to a divalent heteroaryl group, as defined herein, substituted with an aryl group.

The term “biaryl” refers to an aryl group as defined herein substituted with another aryl group as defined herein, including where the aryl groups are independently optionally substituted as described in the definition.

The term “biarylene” refers to a divalent biaryl group, as defined herein.

The term “biarylalkyl” refers to an alkyl group substituted with an aryl group which is substituted with another aryl group, each as defined herein, including where each aryl independently and alkyl are optionally substituted as described in their respective

### Definitions

The terms “heterocyclic,” “heterocyclic ring,” “heterocyclyl,” and “heterocycle” refer to a monocyclic or bicyclic aromatic ring or a saturated or unsaturated, monocyclic or bicyclic ring that is not aromatic comprising 3 to 12 carbon atoms and 1 to 4 heteroatoms independently selected from the group consisting of oxygen, nitrogen, P(═O), and S(O)ₘ within the ring, wherein m is an integer from 0 to 2. In addition to any group specifically recited in any of the embodiments or claims, the heterocyclic ring is optionally substituted with one, two, or three halo, C(═O)OR^{a}, OC(═O)R^{a}, OC(═O)NR^{a}R^{b}, —C₁₋₂₀alkyl, —OH, —NH₂, —OC₁₋₂₀alkyl, —S(O)ₘC₁₋₂₀alkyl (wherein m is 0, 1, or 2), —C₁₋₂₀alkyl-NH₂, —NHC₁₋₄alkyl, —C(═O)H, or —C═N—OR_{d} wherein each R^{a}, R^{b} and R_{d} is independently hydrogen or C₁₋₂₀alkyl. In some embodiments, the heterocyclic ring is unsubstituted. In some or any embodiments, the 4 to 7 or 5 to 7 membered ring formed by any two of R⁴ through R¹⁰ and/or formed by R¹¹ and R¹² and/or formed by R⁴ and R¹¹ and/or formed by R⁶ and R¹² is optionally substituted as described herein for heterocycle. In some or any embodiments, the 5 to 7 membered ring formed by R¹¹ and R¹² and/or formed by R⁴ and R¹¹ and/or formed by R⁶ and R¹² is optionally substituted with one or two groups independently selected from C₁. 6alkyl and aryl.

The term “heterocyclylene” refers to a divalent heterocyclyl group, as defined herein.

The term “unsaturated” in the context of the term cycloalkyl, cycloalkylene, and heterocycle refers to a partially unsaturated, but not aromatic ring.

In some embodiments, heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one, azabicyclo[3.1.0]hexane and the like, and N-oxides of said nitrogen heterocycles. In addition to any group specifically recited in any of the embodiments or claims, heterocyclic rings include substituted and unsubstituted rings, including those substituted with groups selected from C(═O)OR^{a}, OC(═O)R^{a}, OC(═O)NR^{a}R^{b} where each R^{a} and R^{b} are independently hydrogen or C₁₋₆alkyl.

The term “heteroaryl” refers to a five- (5) or six- (6) membered C- or N-linked heterocyclic ring, optionally fused to a benzene or to another heterocyclic ring (wherein at least one of the heterocyclic rings is aromatic). Heterocyclic ring fused to a benzene ring is also referred to as benzo-heterocyclic group. In some embodiments, heteroaryl includes, but is not limited to, pyridine, thiophene, furan, pyrazole, indole, benzimidazole, quinoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4-oxadiazole, 4-oxo-2-thiazolinyl, or 5-methyl-1,3,4-thiadiazol-2-yl, thiazoledione, 1,2,3,4-thiatriazole, and 1,2,4-dithiazolone. In addition to any group specifically recited in any of the embodiments or claims, heteroaryl groups include substituted and unsubstituted rings, including those substituted with groups selected from C(═O)OR^{a}, OC(═O)R^{a}, and OC(═O)NR^{a}R^{b} where each R^{a} and R^{b} are independently hydrogen or C₁₋₆alkyl. In some embodiments, heteroaryl is unsubstituted. Heteroaryl groups distinguished as “heteroaryl” and “heteroaryl¹” or “heteroaryl²” refer to independently selected heteroaryl groups that may be different from each other, or independently equal to each other. If the term “heteroaryl” is used more than once in the same group, then each “heteroaryl” is independent of another “heteroaryl,” at each appearance.

The term “heteroarylalkyl” refers to an alkyl group substituted with an heteroaryl group, each as defined herein.

The term “mono-substituted” refers to a group having at least one substituent in said group, not counting the point of connection of this group to the main structure or general formula. The term “multiply-substituted” refers to a group having at least two substituents in said group, not counting the point of connection of this group to the main structure or general formula.

Unless specified otherwise, “carbon atom” means the atom of element carbon optionally substituted with H, halo, NR^{a}R^{b}, C₁₋₁₂alkyl, C₃₋₇ cycloalkyl, aryl, heteroaryl, or with a heterocyclic ring. Carbon atom comprises atoms with sp3, sp2, and sp electronic hybridization.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds provided herein may possess one or more asymmetric centers; such compounds can therefore be produced as individual (R)- or (S)-stereoisomers or as mixtures thereof. Unless indicated otherwise, the description or naming of a particular compound in the specification and Claims is intended to include all individual enantiomers and any mixtures, racemic, partially racemic, or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A hydrogen (H), carbon (C), or nitrogen (N) substitution for compounds of the formulas I-V include a substitution with any isotope of the respective atom. Thus, a hydrogen (H) substitution includes a ¹H, ²H (deuterium), or ³H (tritium) isotope substitution, as may be desired, for example, for a specific therapeutic or diagnostic therapy, or metabolic study application, or stability enhancement. Optionally, a compound of this invention may incorporate a known in the art radioactive isotope or radioisotope, such as any number of ³H, ¹⁵O, ¹²C, or ¹³N isotopes, to afford a respective radiolabeled compound of formulas I-V.

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating”, “treatment”, or “therapy” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Leaving group” has the meaning conventionally associated with it in synthetic organic chemistry, i.e., an atom or group capable of being displaced by a nucleophile and includes halogen, C₁₋₄alkylsulfonyloxy, ester, or amino such as chloro, bromo, iodo, mesyloxy, tosyloxy, trifluorosulfonyloxy, methoxy, N,O-dimethylhydroxyl-amino, and the like.

“Prodrug” means any compound which releases an active parent drug according to a compound provided herein in vivo when such prodrug is administered to a mammalian subject. Prodrugs of a compound of provided herein are prepared by modifying functional groups present in a compound provided herein in such a way that the modifications may be cleaved in vivo to release the parent compound. Prodrugs include compounds provided herein wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl) of hydroxy functional groups in compounds provided herein, and the like. Prodrugs of compounds provided herein may be used for particular therapeutic application, such as for pulmonary delivery of an aerosol containing a prodrug of such a compound, or to improve tolerance to same agent. For example, a methanesulfonate prodrug form of polymyxin drug colistin (described, for example by Bergen et al. in Antimicrob. Agents Chemother. 2006, vol. 50, p. 1953) is used to reduce neurotoxic effects of colistin, and is used for aerosol administration of this drug. This and other known forms of prodrugs could be likewise used to further improve pharmaceutical properties of the compounds provided herein.

The term “mammal” refers to all mammals including humans, livestock, and companion animals.

The compounds described herein are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “rt” for room temperature).

### Illustrative Embodiments

Within the broadest definition of the present invention, certain compounds of the compounds of formula I may be preferred. Specific and preferred values listed below for radicals, substituents, and ranges, are for illustration only; they do not exclude other defined values or other values within defined ranges for the radicals and substituents.

In some preferred compounds described herein C₁₋₁₄alkyl can be methyl, ethyl, propyl, isopropyl, butyl, iso-butyl, sec-butyl, octyl, nonyl, decyl, and isomeric forms thereof.

In some preferred compounds described herein C₂₋₁₂alkenyl can be vinyl, propenyl, allyl, butenyl, and isomeric forms thereof (including cis and trans isomers).

In some preferred compounds described herein C₃₋₇cycloalkyl can be cyclopropyl, cyclobutyl, cyclopentyl, cyclohexyl, and isomeric forms thereof.

In some preferred compounds described herein C₁₋₁₄heteroalkyl can be hydroxymethyl, hydroxyethyl, 2-(N,N-dimethylamino)ethyl, 2-(4-morpholino)ethyl, and 2-methoxyethyl.

In some preferred compounds described herein halo can be fluoro (F) or chloro (Cl).

It will also be appreciated by those skilled in the art that compounds described herein may have additional chiral centers and be isolated in optically active and racemic forms. The present invention encompasses any racemic, optically active, tautomeric, geometric, or stereoisomeric form, or mixture thereof, of a compound of the invention.

Any embodiment described herein can be combined with any other embodiment described herein.

Embodiment 2: The compound of formula I-P, as provided above, and wherein

the integers a through g are all equal to 1; and wherein

A¹ is Thr or Ser; A², A³ A⁶, and A⁷ are independently selected from Dab, Dap, Ser, and Thr; A⁴ is Leu or Ile; and A⁵ is Phe, D-Phe, Bip, D-Bip, Val, or D-Val.

Embodiment 3: The compound of formula I-P or of embodiment 2, and wherein a cyclic peptide structure comprised of optional amino acid residues A¹ through A⁷ is a cyclic peptide structure identical to that present in the natural products polymyxin B, polymyxin E, or octapeptin, or similar structures present in SPR206 and SPR741.

Embodiment 4: The compound of formula I-P or of any one of embodiments 2 and 3 according to formula II-P

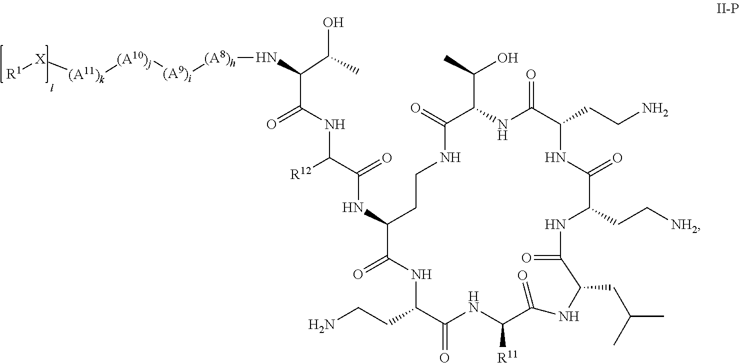

or a pharmaceutically acceptable salt, solvate, or hydrate thereof, wherein:

R¹¹ is CH₂CH(CH₃)₂ or CH₂Ph; and

R¹² is CH₂NH₂ or CH₂CH₂NH₂.

Embodiment 5: The compound of formula II-P of embodiment 4, wherein group X in the formula II-P is selected from the structures below, wherein either the left side or the right side of groups X depicted below is connected to respective group R therein:

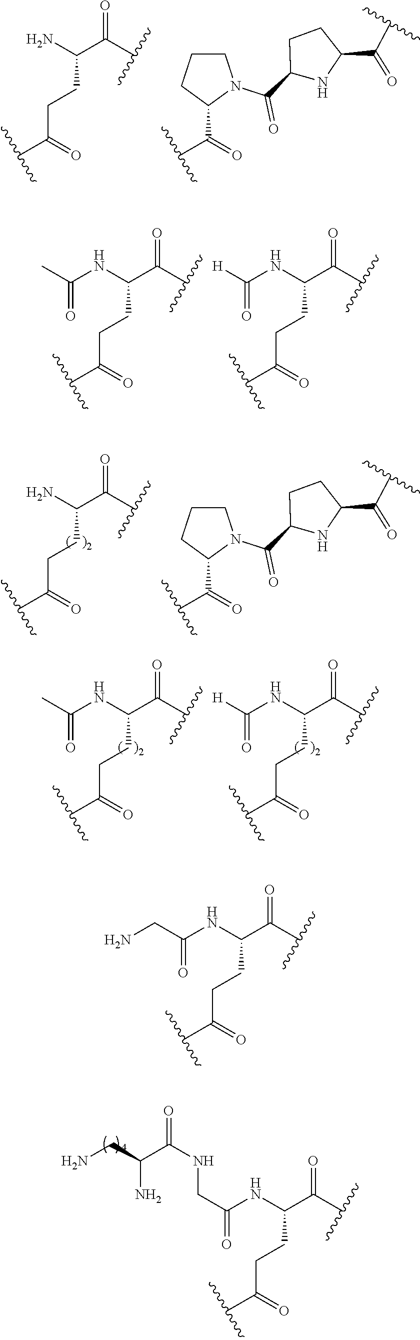

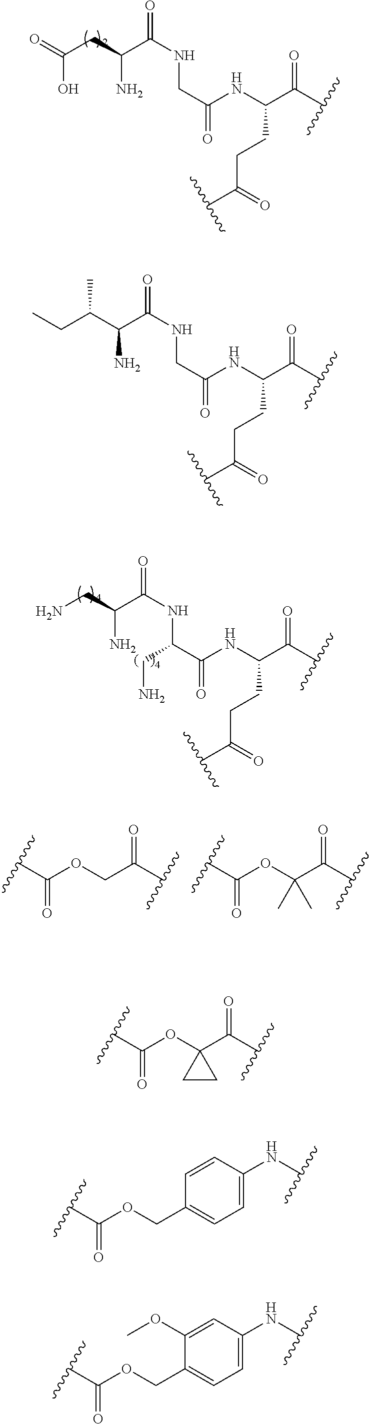

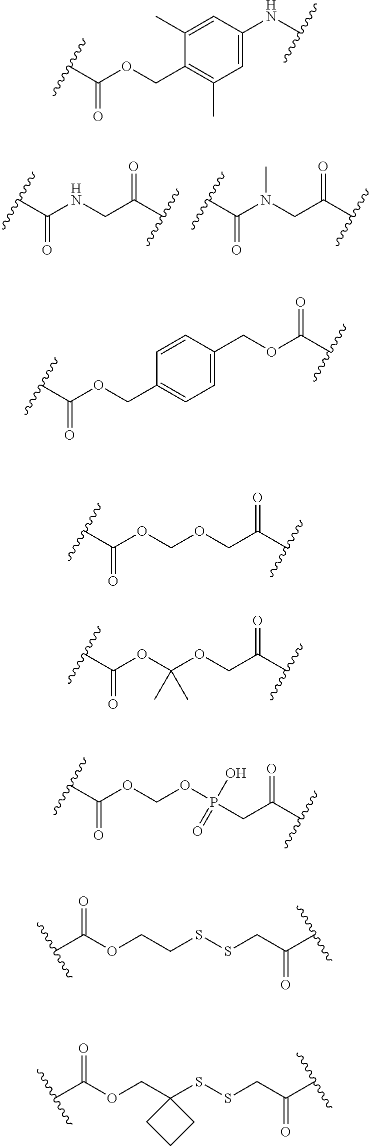

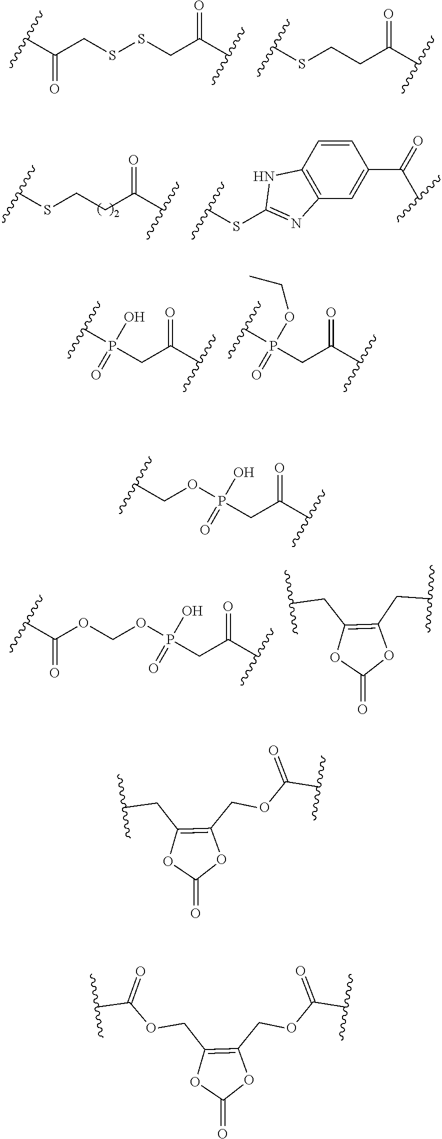

Embodiment 6: The compound of the formula II-P of embodiment 5, wherein group X, either at its left or right side therein, incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂-)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂-)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂-)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂-)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂-)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), and similar linear groups.

Embodiment 7: The compound of formula I-P or of any one of embodiments 2 and 3 according to formula III-P

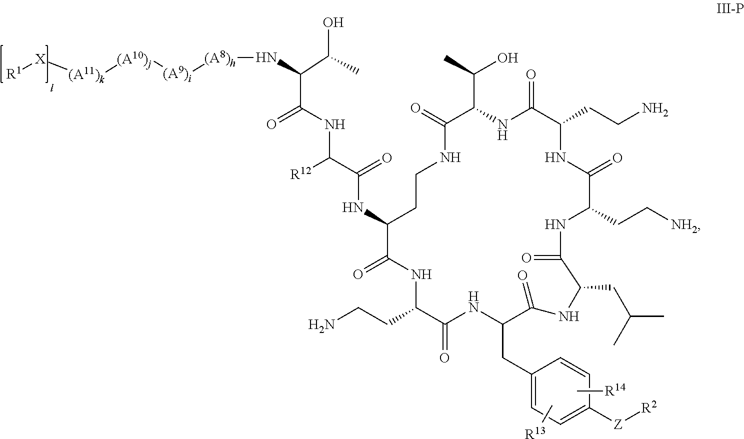

wherein groups R¹³ and R¹⁴ are independently selected from H, halo, NH₂, CN, OH, OC₁₋₁₄alkyl, Oaryl, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl, C(═O)OH, C₁₋₁₄alkylC(═O)OH, and C₁₋₁₄alkylC(═O)—OC₁₋₁₄alkyl.

Embodiment 8: The compound of the formula III-P of embodiment 7, wherein group Z in the formula II is selected from the structures below, wherein the right side of groups Z depicted below is connected to respective group R² therein:

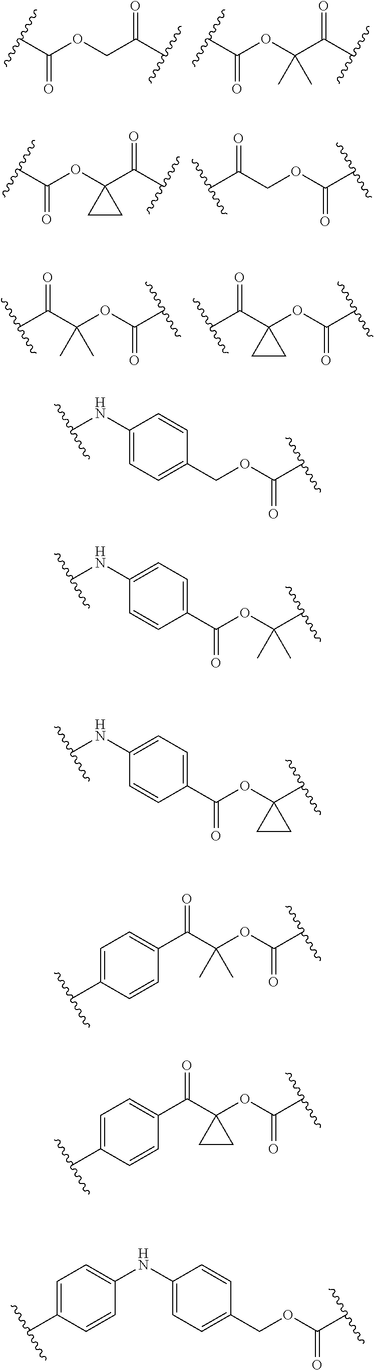

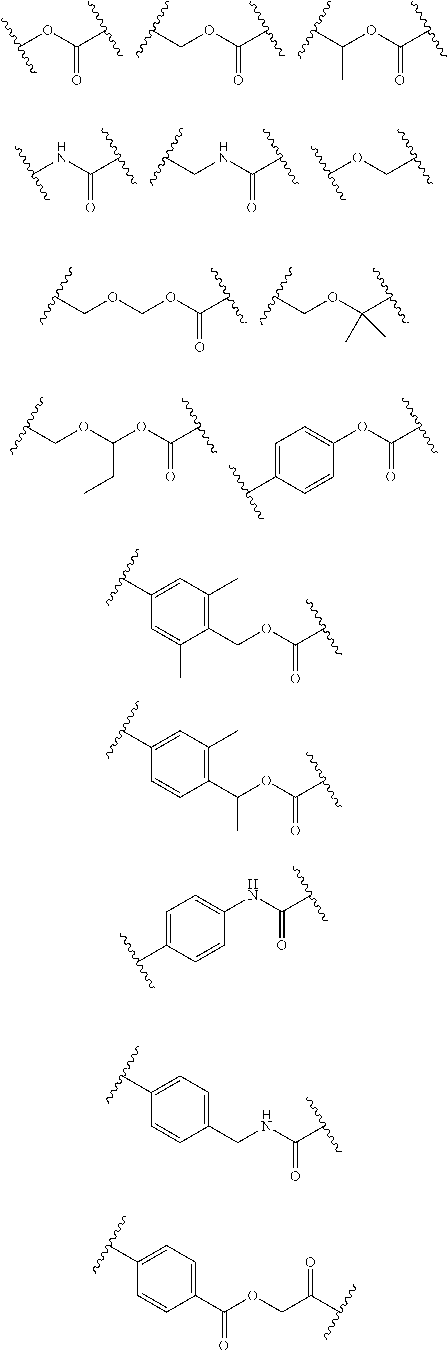

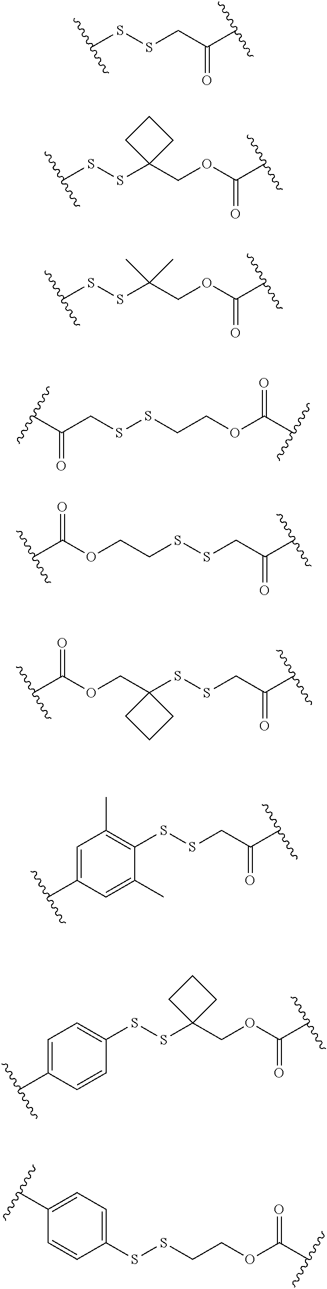

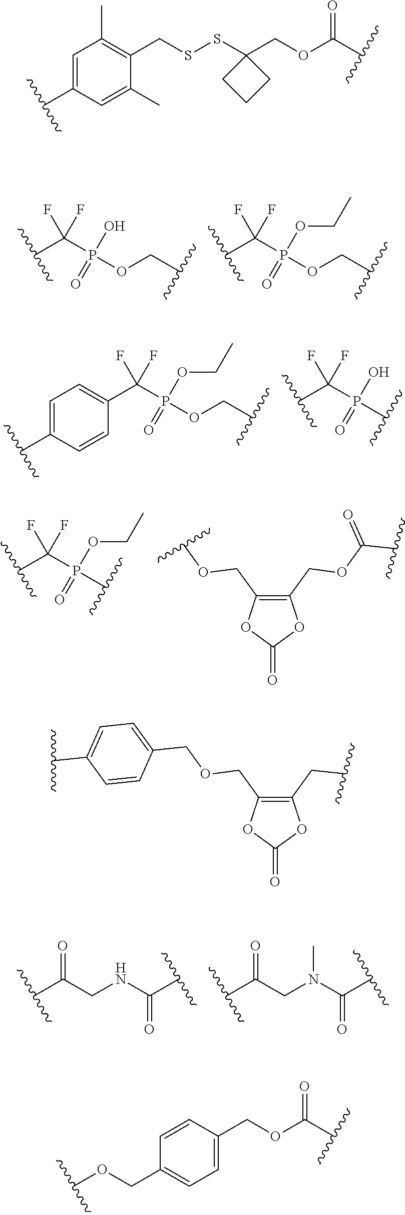

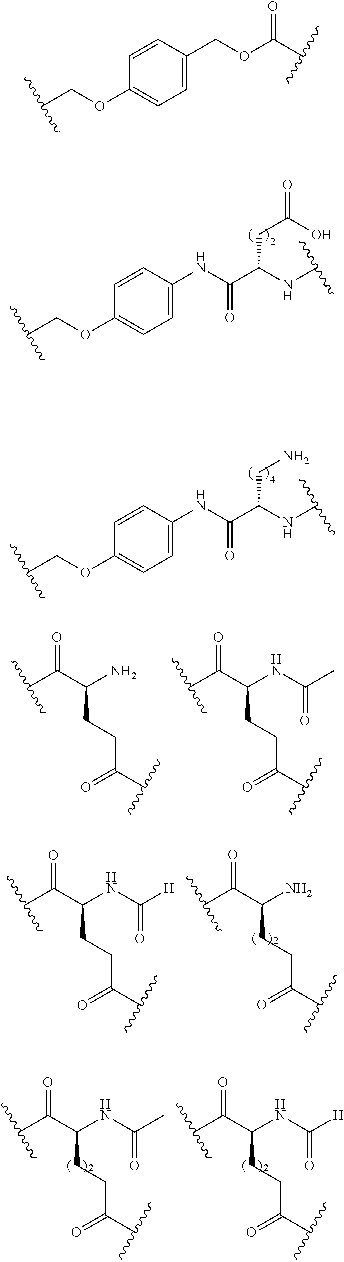

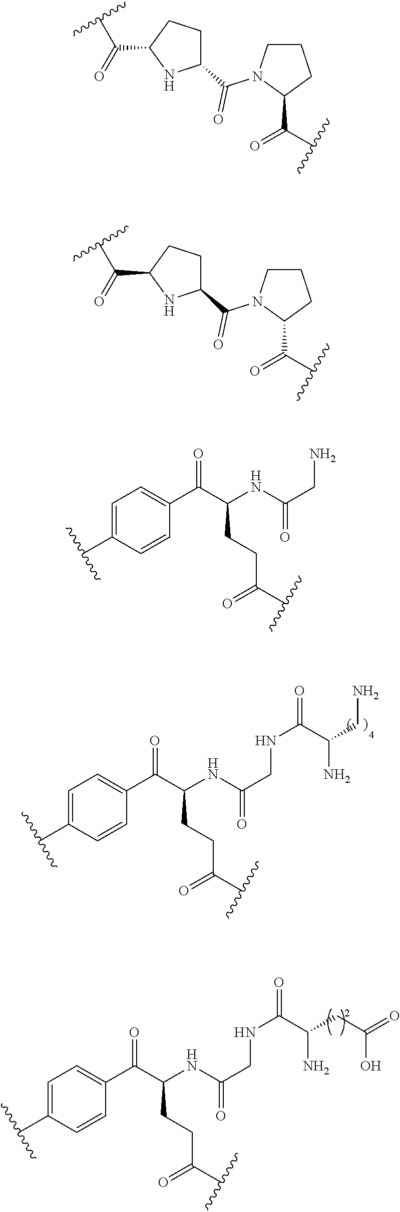

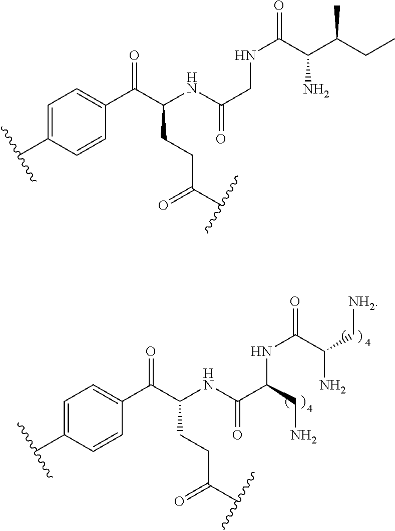

In one embodiment is provided a compound of formula I of the following formula II:

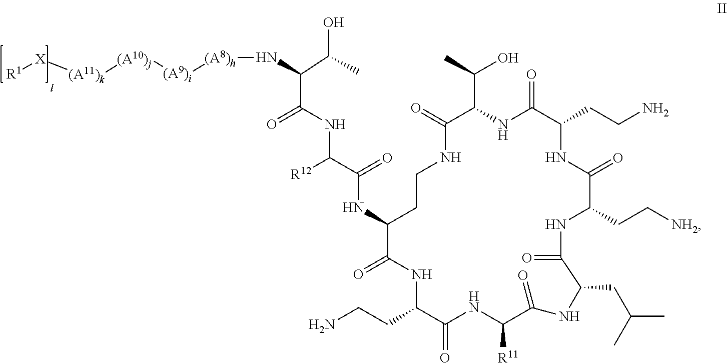

or a pharmaceutically acceptable salt, solvate, or hydrate thereof, wherein R¹¹ is CH₂CH(CH₃)₂ or CH₂Ph; and R¹² is CH₂NH₂ or CH₂CH₂NH₂; and wherein other groups and integers in a compound of formula II are selected just as defined above for a compound of formula I, or any embodiment thereof.

One preferred group of compounds of the formulas I or II is illustrated below, wherein each X in the formula II is independently selected from the structures below, wherein either the left side or the right side of X depicted below is connected to its respective R¹ therein:

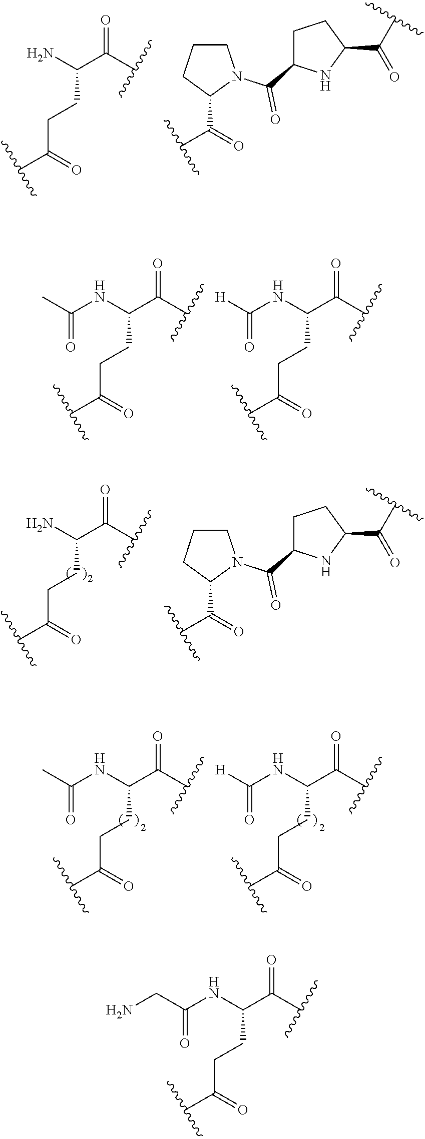

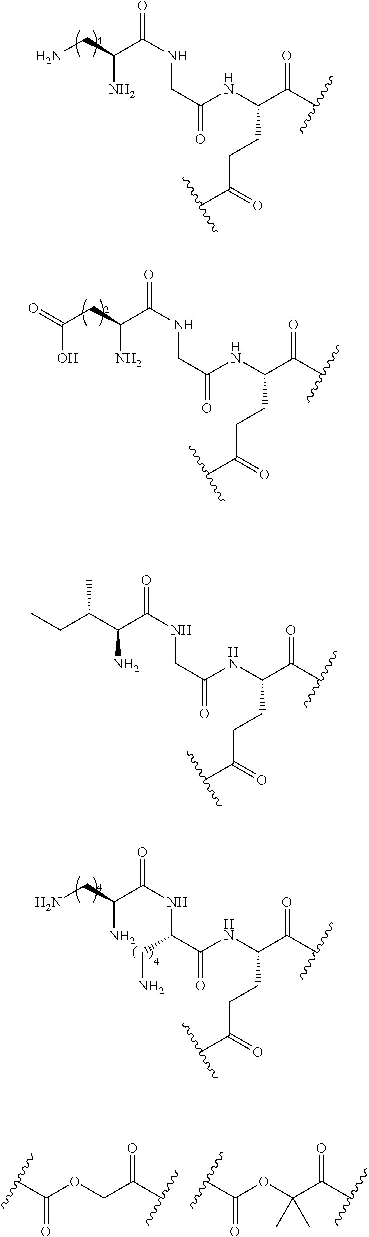

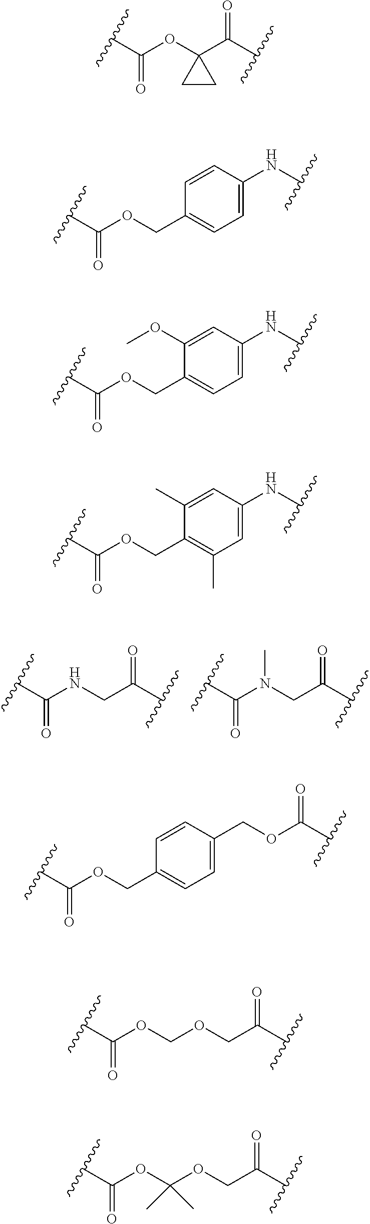

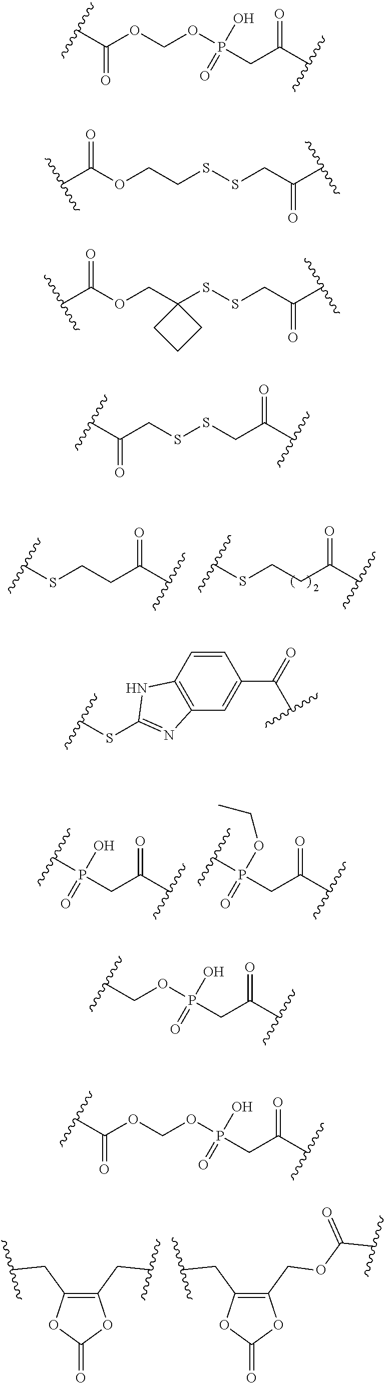

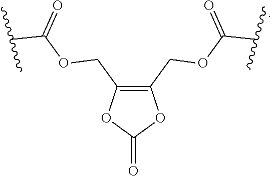 [SMILES: *C(=O)OCc1oc(=O)oc1COC(*)=O]

In another embodiment, provided is a compound of formula II wherein each X in formula II is independently selected from the following structures, connected to R¹ at the left side of X below:

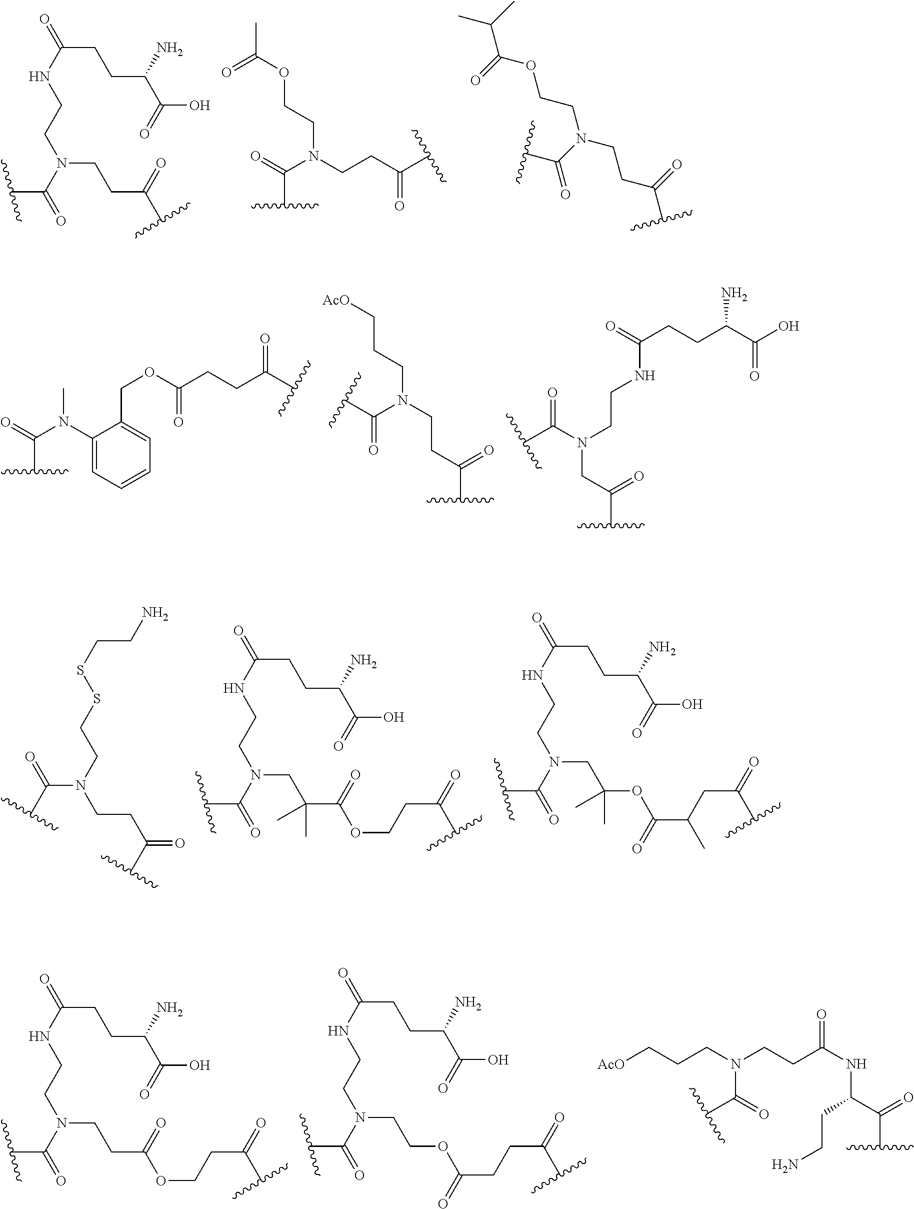

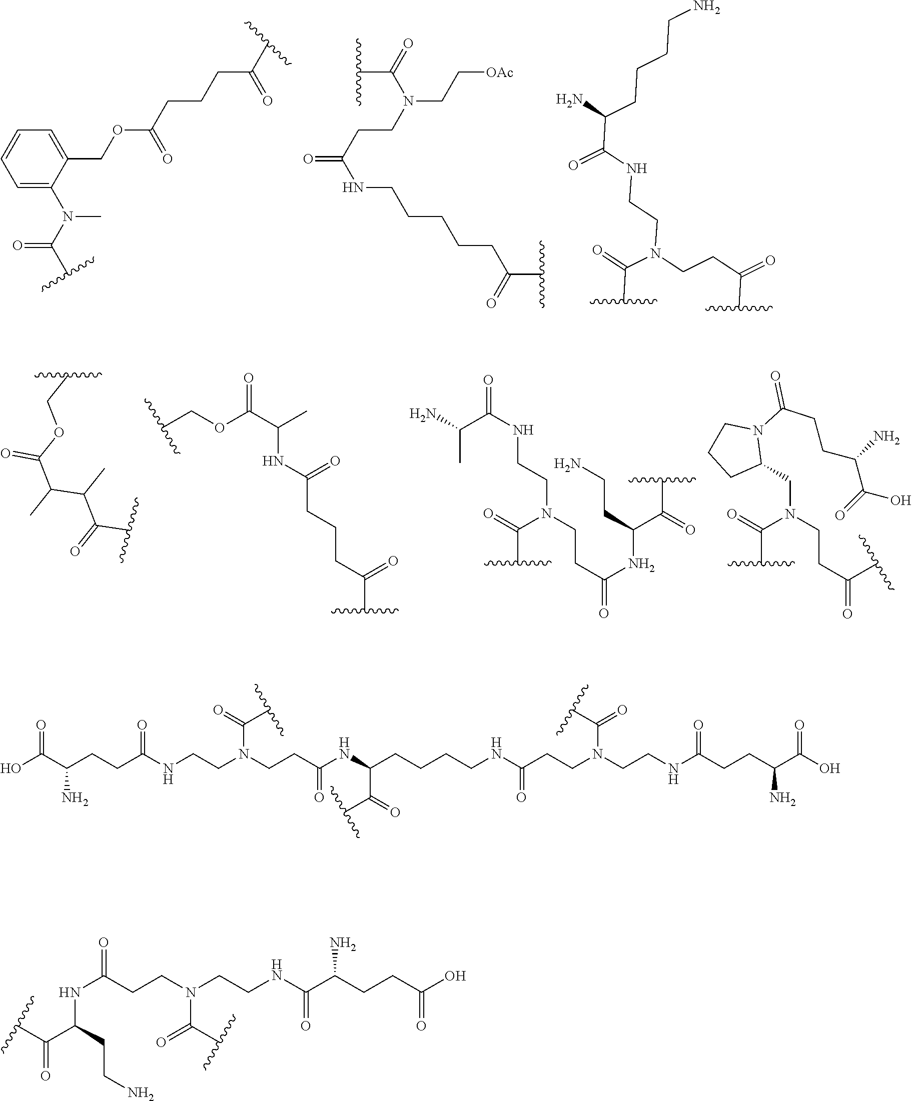

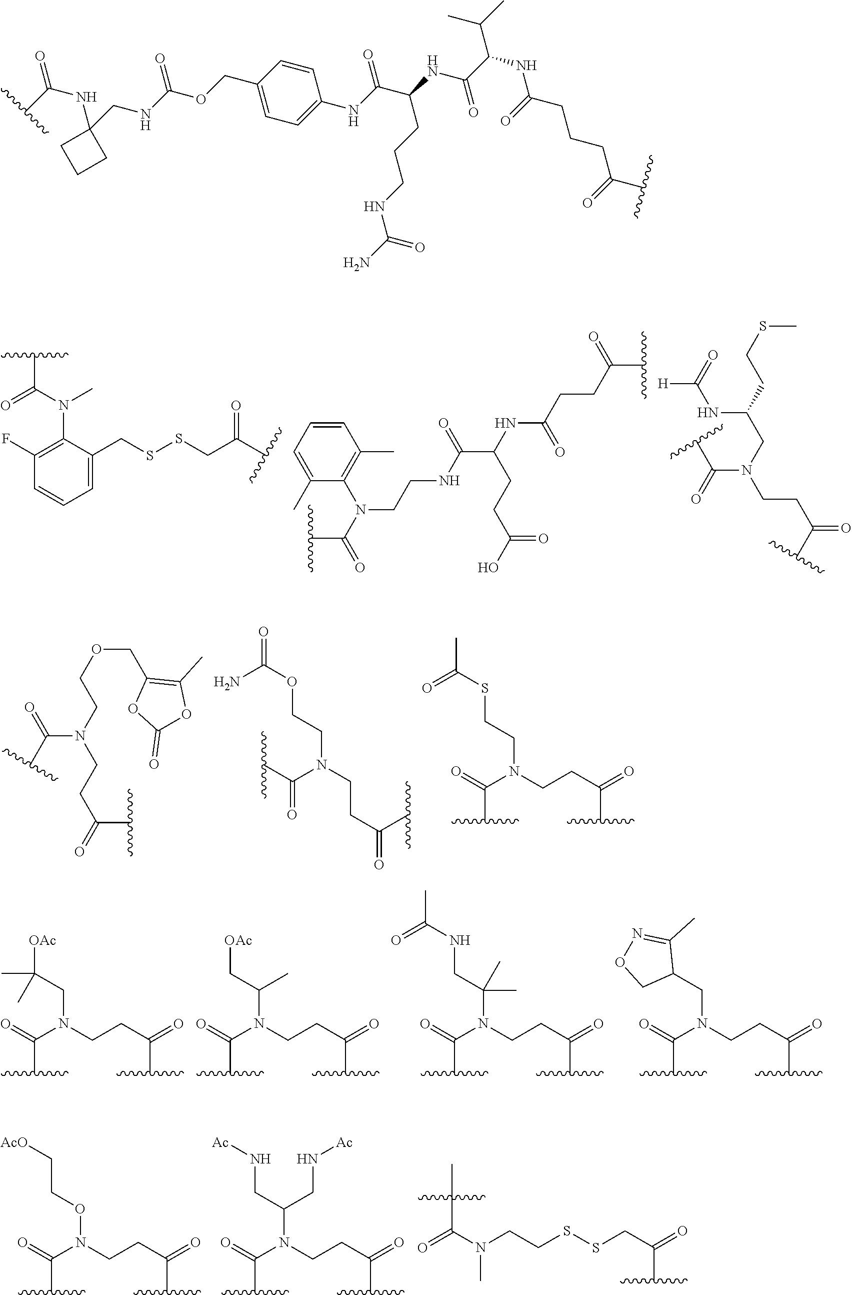

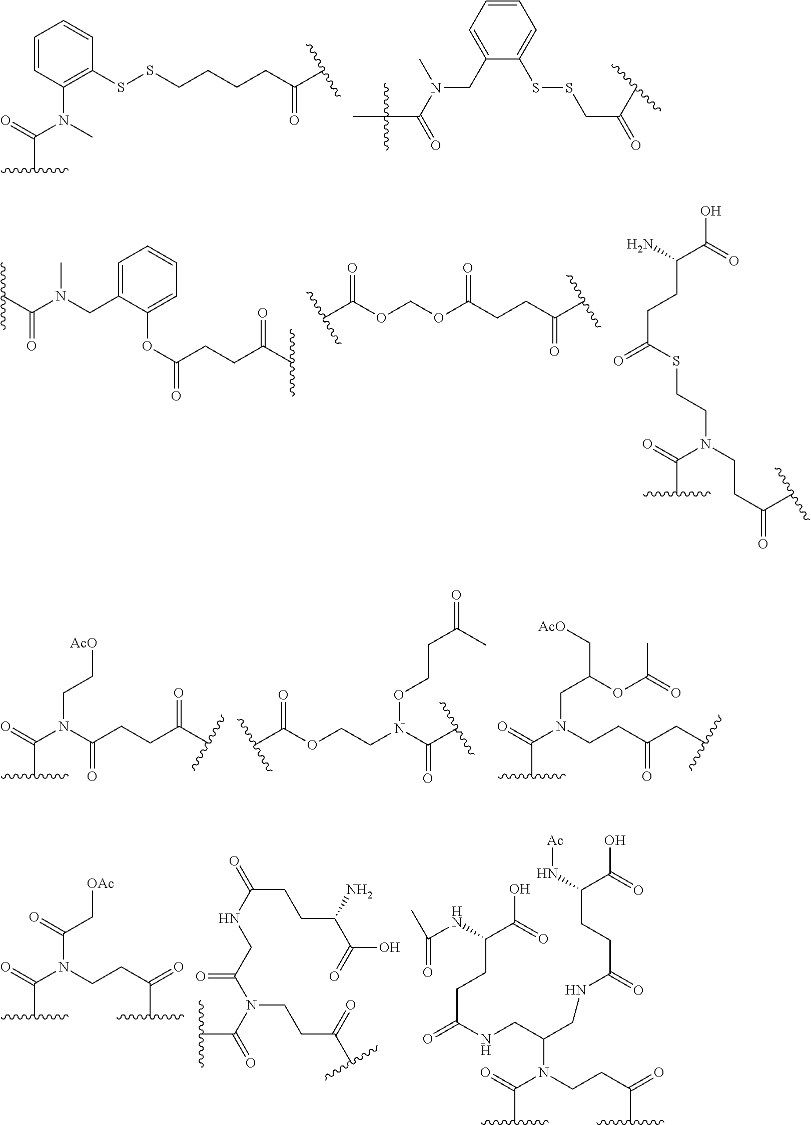

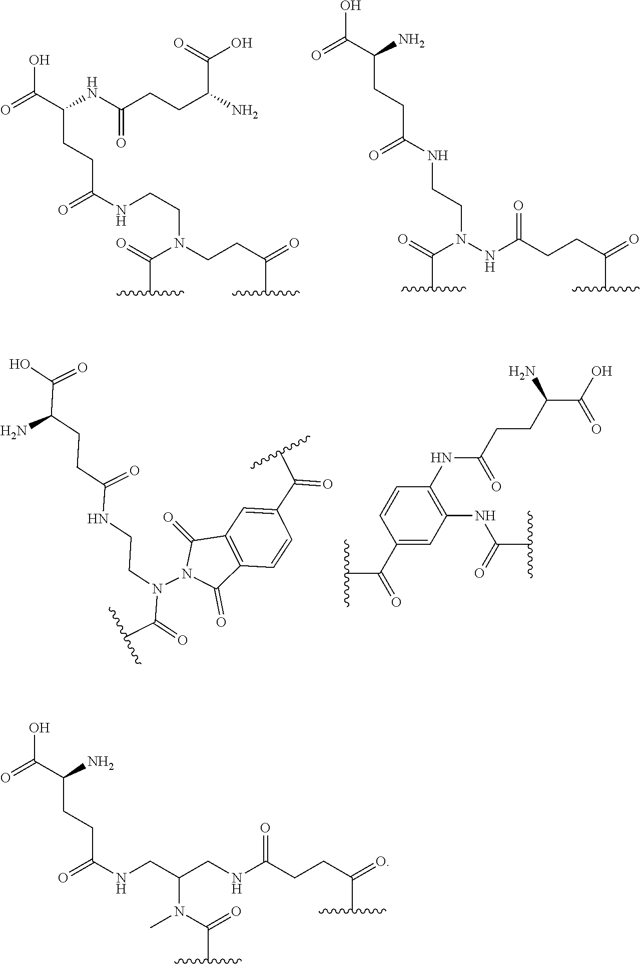 [SMILES: *C(=O)CCN(CCNC(=O)CC[C@@H](NC(=O)CC[C@@H](N)C(=O)O)C(=O)O)C(*)=O ; *C(=O)CCC(=O)NN(CCNC(=O)CC[C@H](N)C(=O)O)C(*)=O ; *C(=O)c1ccc2c(c1)C(=O)N(N(CCNC(=O)CC[C@@H](N)C(=O)O)C(*)=O)C2=O ; *C(=O)Nc1cc(C(*)=O)ccc1NC(=O)CC[C@@H](N)C(=O)O ; *C(=O)CCC(=O)NCC(CNC(=O)CC[C@H](N)C(=O)O)N(C)C(*)=O]

In another preferred embodiment, each X illustrated in the two paragraphs above, either at its left or right side therein, independently incorporate additional divalent groups independently selected from C₁₋₁₂alkylene, C₂₋₁₂alkenylene, C₂₋₁₂alkynylene, (CH₂-)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₄alkyl)C(═O), O(CH₂)ₚO(CH₂)ᵣO(CH₂)ₛC(═O), O(CH₂)ₚO(CH₂-)ᵣO(CH₂)ₛOC(═O), O(CH₂)ₚO(CH₂)ᵣO(CH₂)ₛNHC(═O), O(CH₂)ₚO(CH₂)ᵣO(CH₂), N(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl)(CH₂)ₚO(CH₂)ᵣO(CH₂)ₛN(C₁₋₁₄alkyl)C(═O), or similar linear groups.

In another embodiment, provided is a compound of formula I of the following formula III:

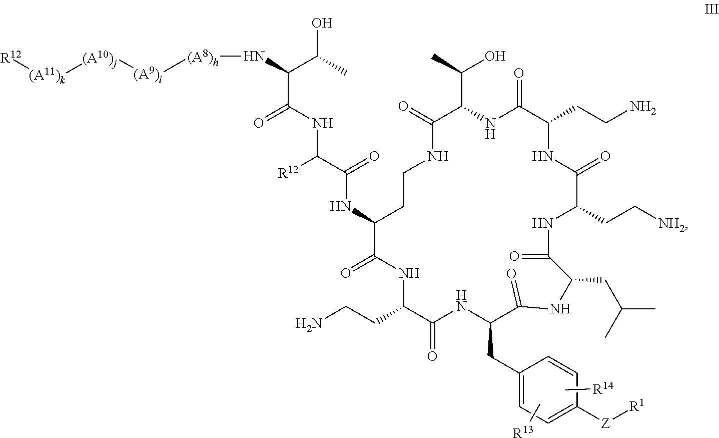

wherein R¹³ and R¹⁴ are independently selected from H, halo, NH₂, CN, OH, OC₁₋₁₄alkyl, Oaryl, NH(C₁₋₆alkyl), NH(OC₁₋₆alkyl), C₁₋₁₄alkyl, C₃₋₆cycloalkyl, aryl, arylalkyl, biaryl, biarylalkyl, heteroarylalkyl, C(═O)OH, C₁₋₁₄alkylC(═O)OH, and C₁₋₁₄alkylC(═O)—OC₁₋₁₄ alkyl; and wherein other groups and integers in a compound of formula III are selected just as defined above for a compound of formula I, or any embodiment thereof.

One preferred group of compounds of formulas I or III, is that wherein Z in formula III is selected from the structures below, wherein the right side of Z depicted below is connected to its respective R²:

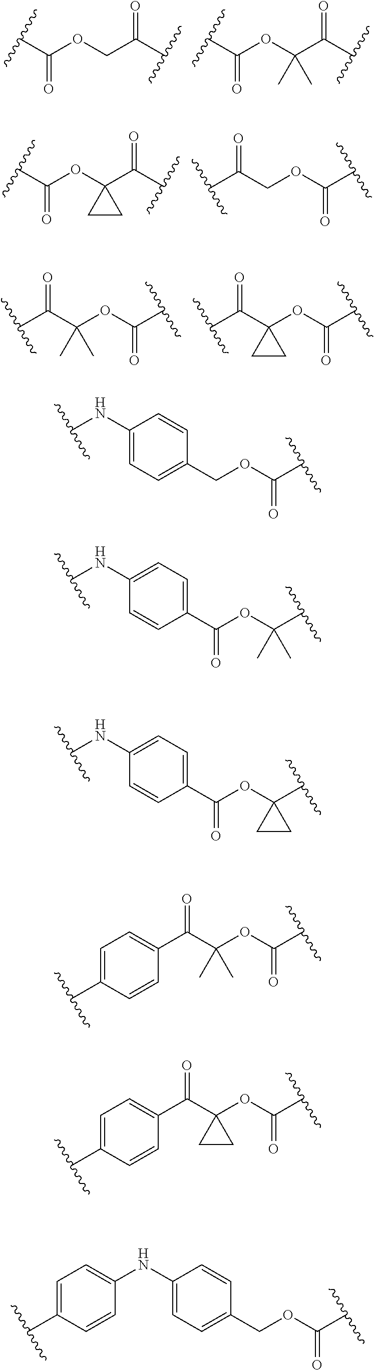

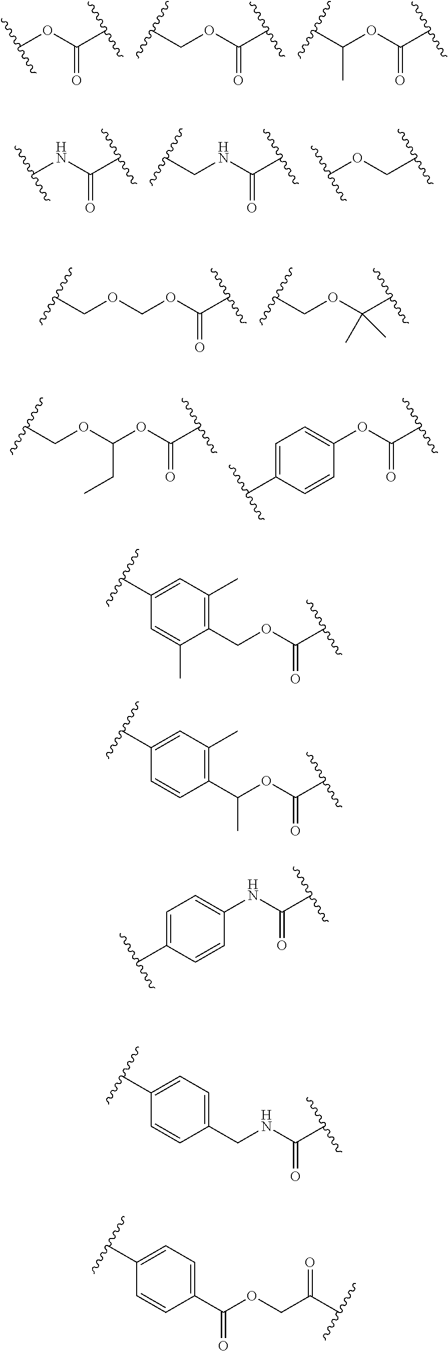

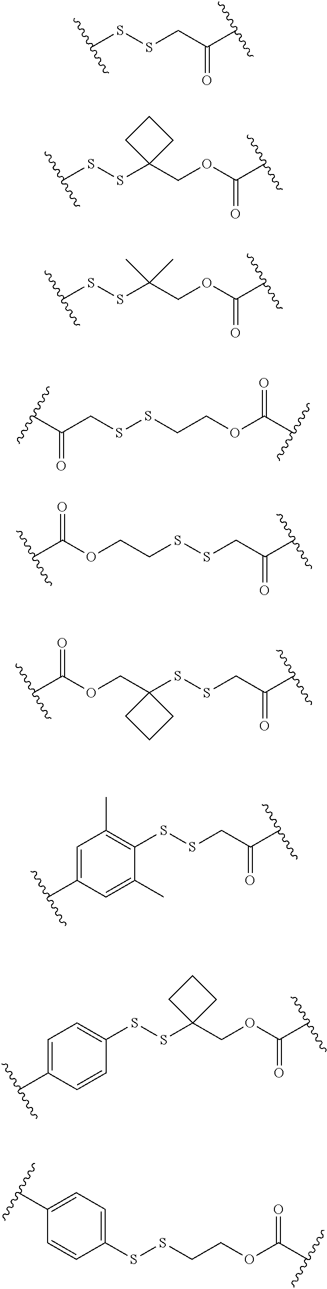

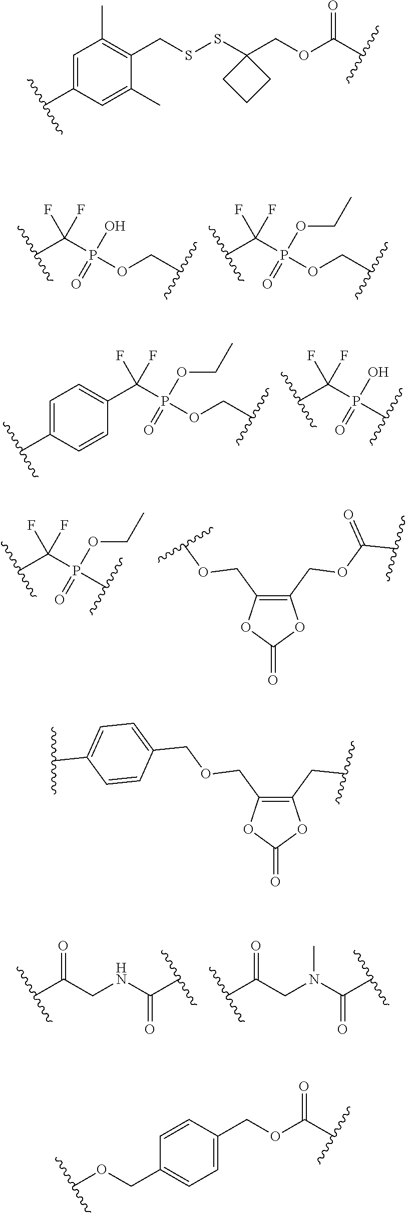

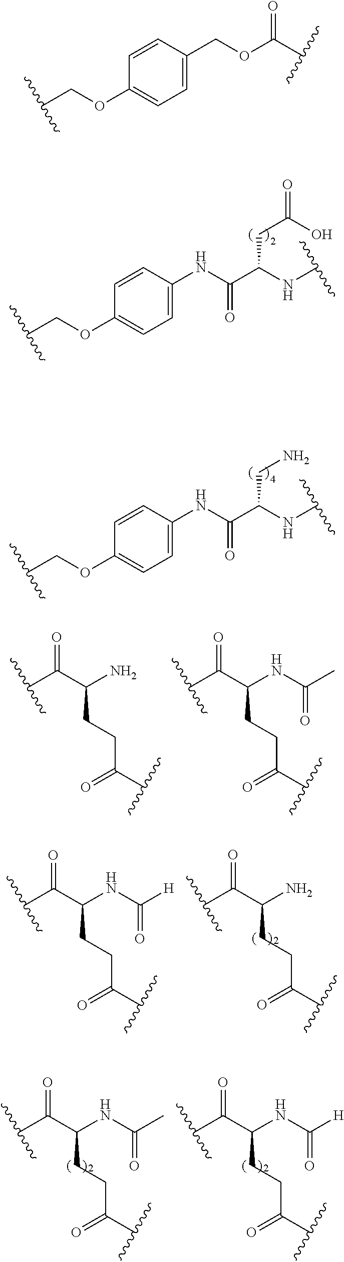

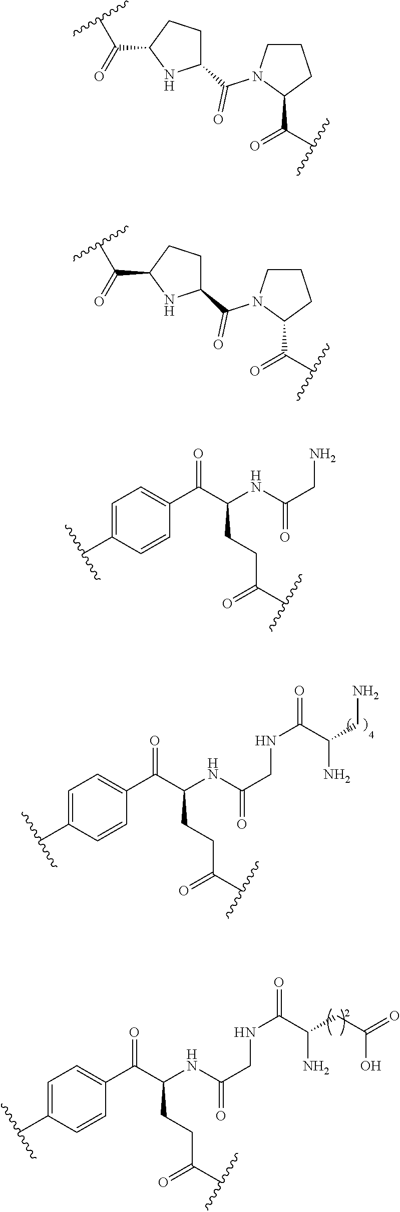

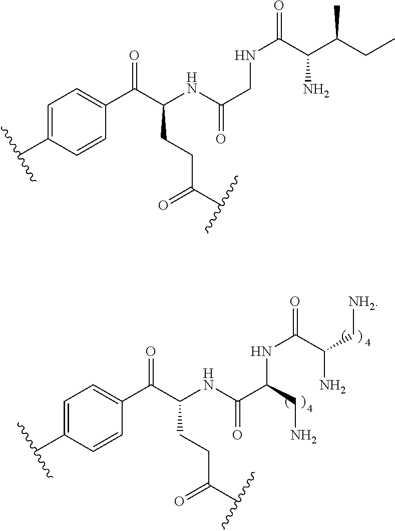

In another preferred embodiment, each Z illustrated above, at its left side therein, incorporate additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)O(CH₂)ₛOC(═O), (CH₂-)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂-)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂-)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂-)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂-)ₚO(CH₂)ᵣO(CH₂)ₛOC(═O), N(C₁₋₁₄alkyl)(CH₂)ₚO(CH₂)ᵣO(CH₂)ₛNHC(═O), N(C₁₋₁₄alkyl(CH₂)ₙO(CH₂)ₚO(CH₂)N(C₁₋₁₄alkyl)C(═O), or similar linear groups.

In another preferred embodiment, provided is a compound of formula I of the following formula IV:

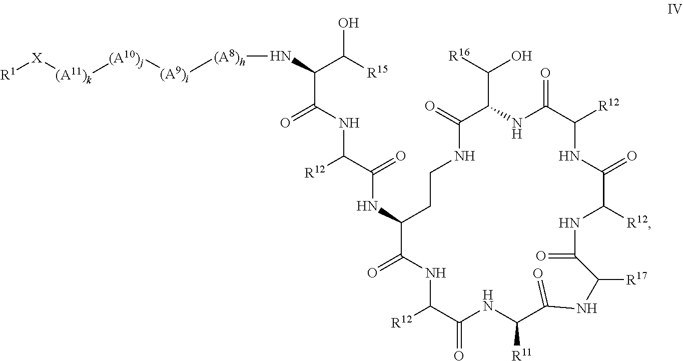

or a pharmaceutically acceptable salt, solvate, or hydrate thereof, wherein
X is selected from the following structures and is connected to R¹ at the left side of X:
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O) (CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)(CR⁹R¹⁰)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O),
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), or
(R)—C(═O)N[CH₂CH₂NHC(═O)CHNH(CH₂CH₂NH₂)]CH₂CH₂C(═O); and
R¹¹ is C₁₋₁₂alkyl, CH(CH₃)₂, CH₂aryl, or CH₂Ph; and
R¹² is CH₂NH₂, CH₂CH₂NH₂, or CH₂CH₂CH₂CH₂NH₂; and
R¹⁵, R¹⁷ and R¹⁷ are independently H, Me, or C₁₋₁₂alkyl.

In another preferred embodiment, provided is a compound formula I or IV, wherein R¹ is selected from the structures below:

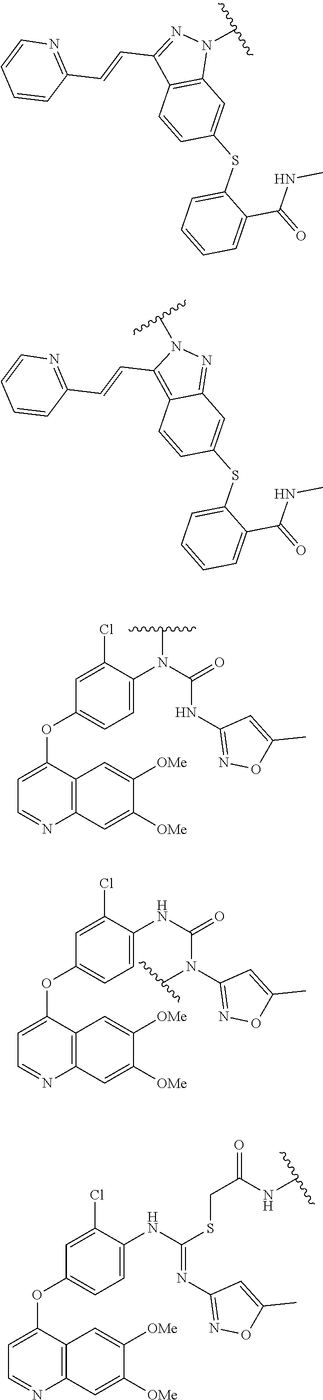 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1 ; *N(C(=O)Nc1cc(C)on1)c1ccc(Oc2ccnc3cc(OC)c(OC)cc23)cc1Cl ; *N(C(=O)Nc1ccc(Oc2ccnc3cc(OC)c(OC)cc23)cc1Cl)c1cc(C)on1 ; *NC(=O)CS/C(=N\c1cc(C)on1)Nc1ccc(Oc2ccnc3cc(OC)c(OC)cc23)cc1Cl]

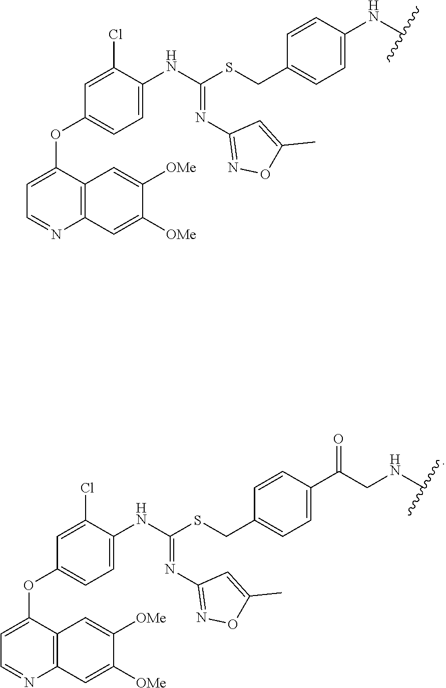 [SMILES: *Nc1ccc(CS/C(=N\c2cc(C)on2)Nc2ccc(Oc3ccnc4cc(OC)c(OC)cc34)cc2Cl)cc1 ; *NCC(=O)c1ccc(CS/C(=N\c2cc(C)on2)Nc2ccc(Oc3ccnc4cc(OC)c(OC)cc34)cc2Cl)cc1]

In another preferred embodiment, provided is a compound formula I or V

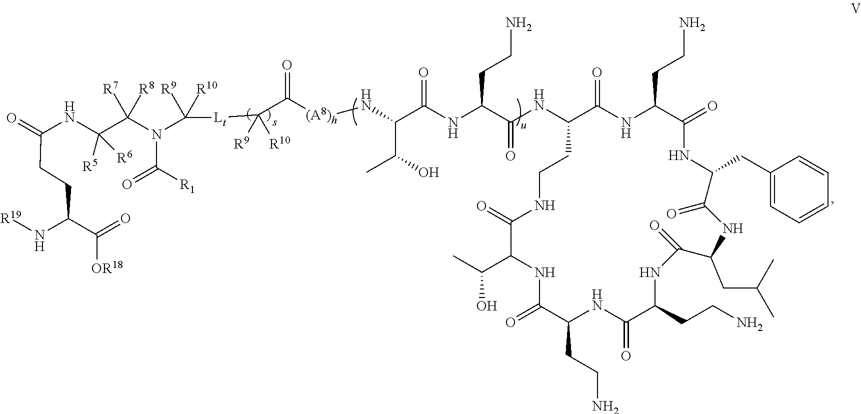

or a pharmaceutically acceptable salt, solvate, or hydrate thereof, wherein
R¹⁸ is H or C₁₋₁₂alkyl; and
   R¹⁹ is H, C₁₋₁₂alkyl, C(═O)C₁₋₁₂alkyl, C(═O)OC₁₋₁₂alkyl, C(═O)OC₁₋₁₂alkyl, C(═O)NHC₁₋₁₂alkyl, SO₂C₁₋₁₂alkyl, SO₂aryl, C(═O)C₃₋₇cycloalkyl, C(═O)OC₃₋₇cycloalkyl, C(═O)NHC₃₋₇cycloalkyl, C(═O)NHC₁₋₁₂alkyl, SO₂C₃₋₇cycloalkyl; and wherein
   each optional group L is selected from CR²⁰R²¹OC(═O)CR²²R²³ and CR²⁰R²¹C(═O)OCR²²R²³; wherein
   R²⁰ through R²³ are independently selected from H, C₁₋₁₂alkyl, or C₃₋₇cycloalkyl; or any of the two adjacent groups R²⁰ and R²¹ or R²² and R²³ independently taken together form a C₃₋₇cycloalkyl group; and
an integer t is 0, 1, or 2; and
an integer u is 0 or 1.

In another preferred embodiment, provided is a compound formula I or V wherein R¹ is selected from structures below:

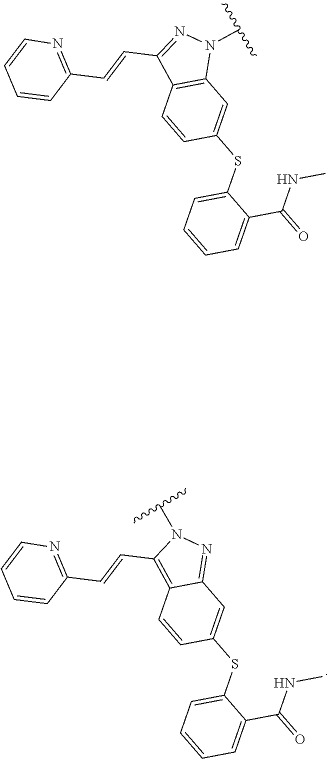 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1]

In another preferred embodiment, the compound of any one of formulas I-P, II-P, III-P, and I-IV, is that where X is C(O)N[(CR⁵R⁶)ₚNHC(O)(CR⁷R⁸)ᵣCH(NH₂)COOH](CR⁹R¹⁰)ₛ C(O).

Some preferred compounds are exemplified below in Table A, wherein, if present, PMBN group is a polymyxin B nonapeptide nonapeptide (H-Thr-Dab-cyclo[Dab-Dab-D-Phe-Leu-Dab-Dab-Thr]) residue incorporated into structures below with a chemical bond formed through the replacement of the H atom at the H-Thr (terminal side chain Thr amino group) of polymyxin B nonapeptide, or any salt or solvate thereof.

| Cpd. No | Structure |
|---|---|
| 1 | 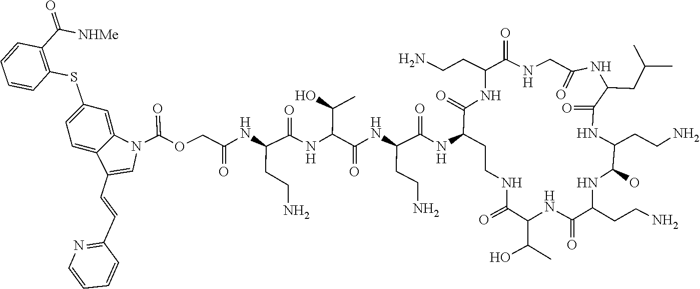 |
| 2 | 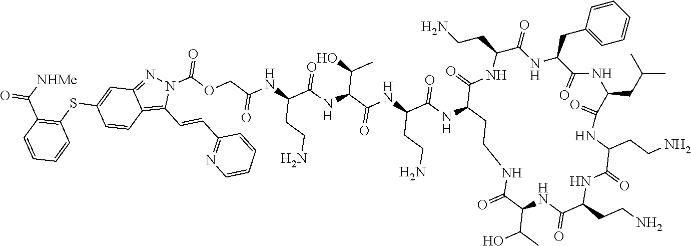 [SMILES: CNC(=O)c1ccccc1Sc1ccc2c(/C=C/c3ccccn3)n(C(=O)OCC(=O)N[C@H](CCN)C(=O)N[C@H](C(=O)N[C@H](CCN)C(=O)N[C@@H]3CCNC(=O)[C@H](C(C)O)NC(=O)[C@H](CCN)NC(=O)C(CCN)NC(=O)[C@H](CC(C)C)NC(=O)[C@H](Cc4ccccc4)NC(=O)[C@H](CCN)NC3=O)[C@H](C)O)nc2c1] |
| 3 | 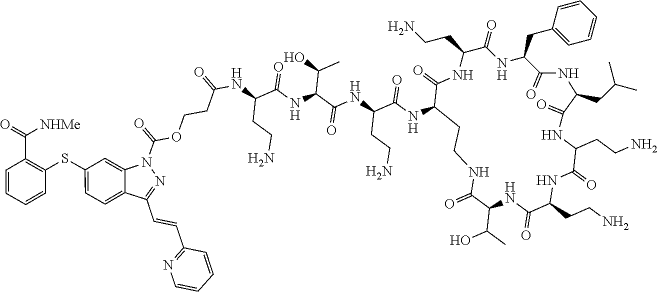 [SMILES: CNC(=O)c1ccccc1Sc1ccc2c(/C=C/c3ccccn3)nn(C(=O)OCCC(=O)N[C@H](CCN)C(=O)N[C@H](C(=O)N[C@H](CCN)C(=O)N[C@@H]3CCNC(=O)[C@H](C(C)O)NC(=O)[C@H](CCN)NC(=O)C(CCN)NC(=O)[C@H](CC(C)C)NC(=O)[C@H](Cc4ccccc4)NC(=O)[C@H](CCN)NC3=O)[C@H](C)O)c2c1] |
| 4 | 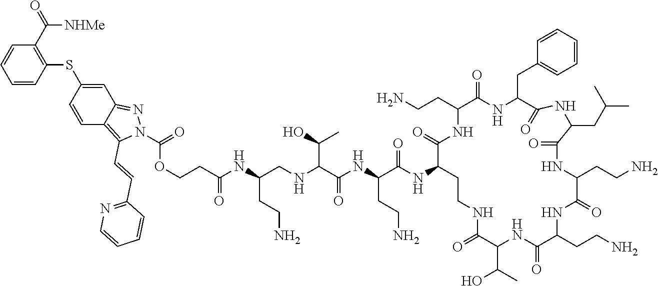 [SMILES: CNC(=O)c1ccccc1Sc1ccc2c(/C=C/c3ccccn3)n(C(=O)OCCC(=O)N[C@H](CCN)CNC(C(=O)N[C@H](CCN)C(=O)N[C@@H]3CCNC(=O)C(C(C)O)NC(=O)C(CCN)NC(=O)C(CCN)NC(=O)C(CC(C)C)NC(=O)C(Cc4ccccc4)NC(=O)C(CCN)NC3=O)[C@H](C)O)nc2c1] |
| 5 | 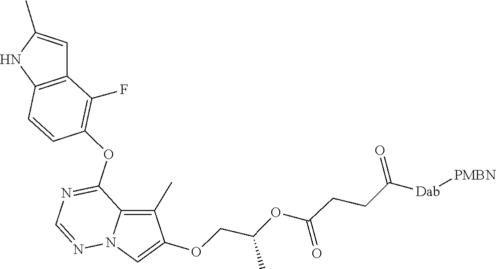 |
| 6 | 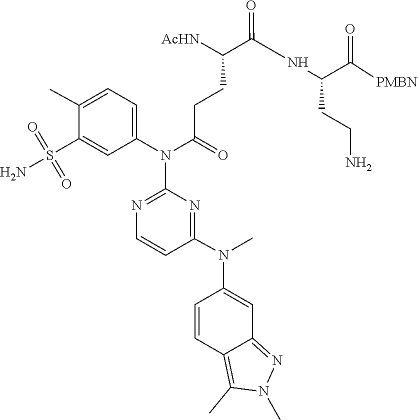 |
| 7 | 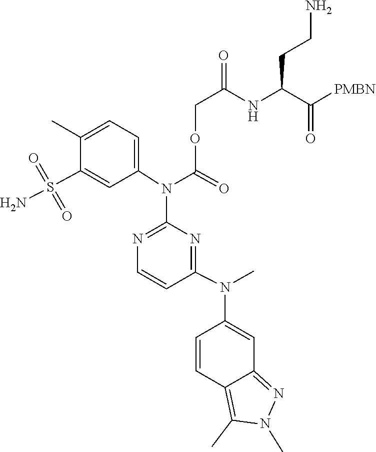 |
| 15 | 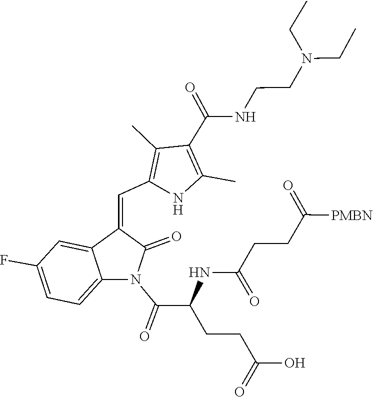 |
| 16 | 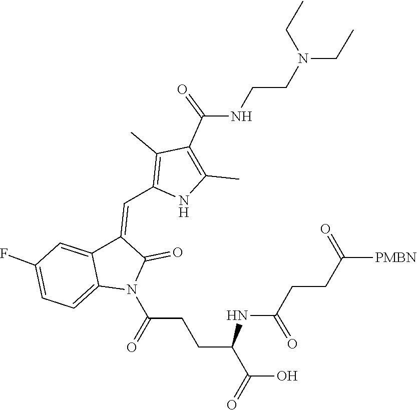 |
| 18 | 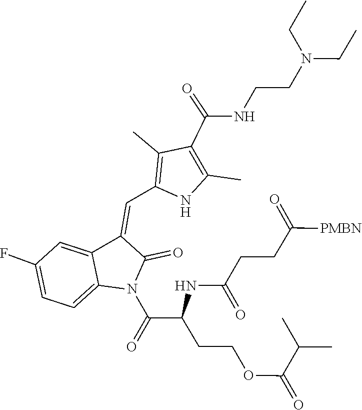 |
| 19 | 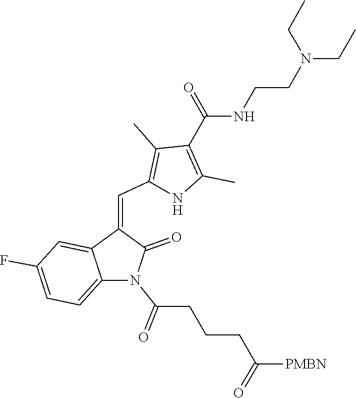 |
| 20 | 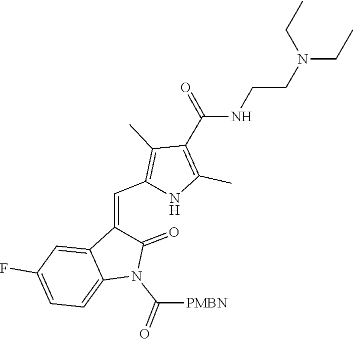 |
| 21 | 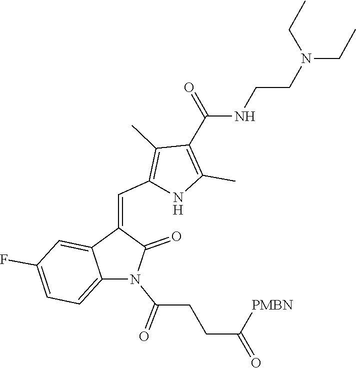 |
| 28 | 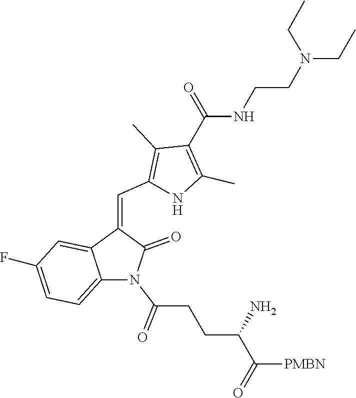 |
| 29 | 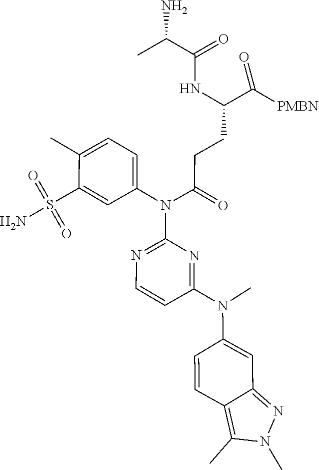 |
| 30 | 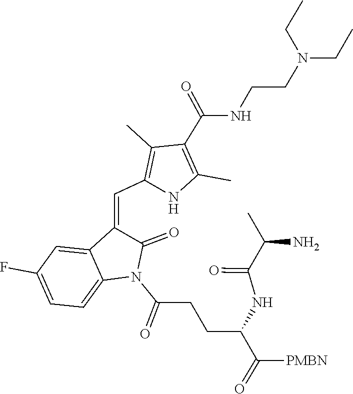 |
| 31 | 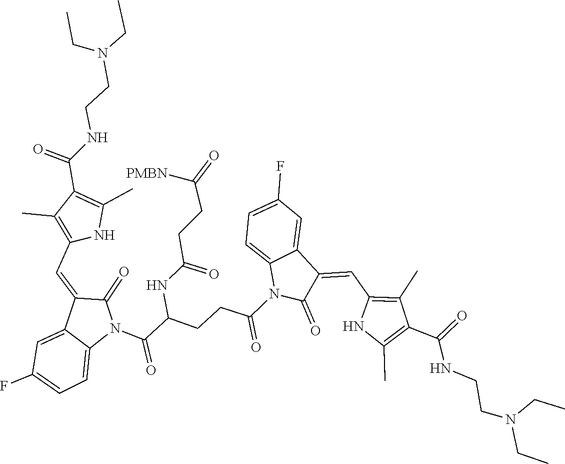 |
| 32 | 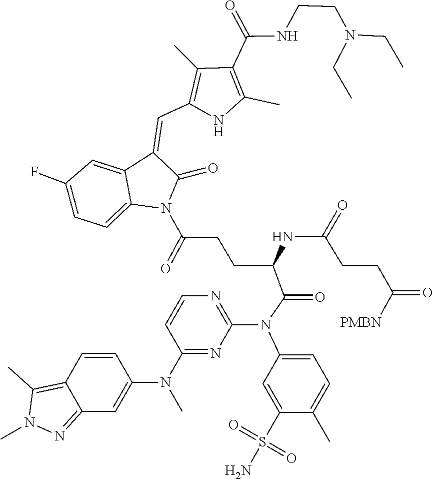 |
| 33 | 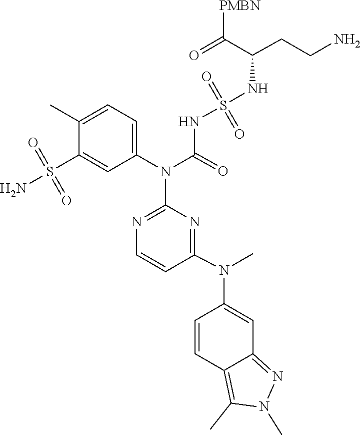 |
| 40 | 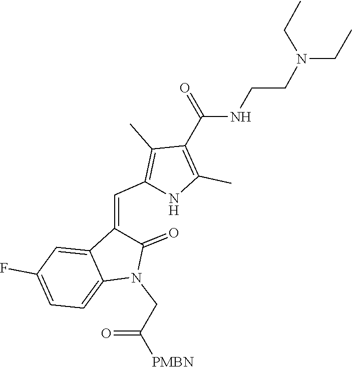 |
| 41 | 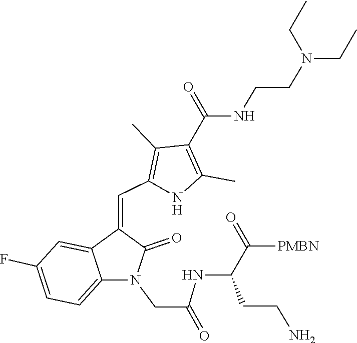 |
| 42 | 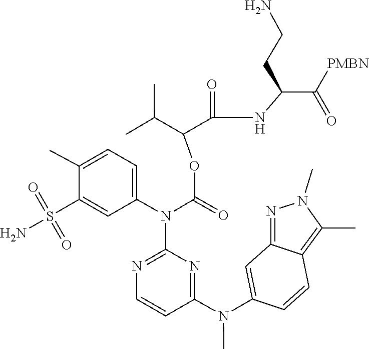 |
| 43 | 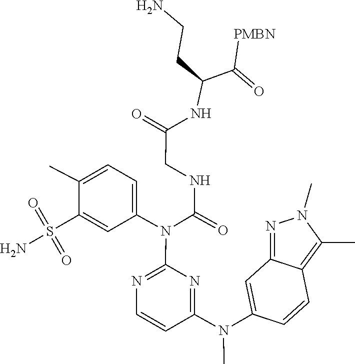 [SMILES: Cc1ccc(N(C(=O)NCC(=O)N[C@H](C=O)CCN)c2nccc(N(C)c3ccc4c(C)n(C)nc4c3)n2)cc1S(N)(=O)=O ; "PMBN"] |
| 44 | 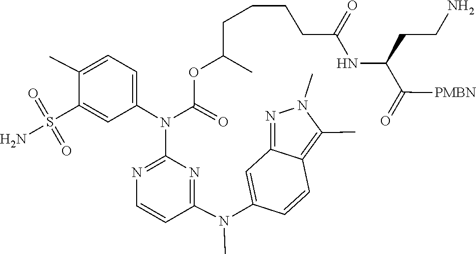 |
| 45 | 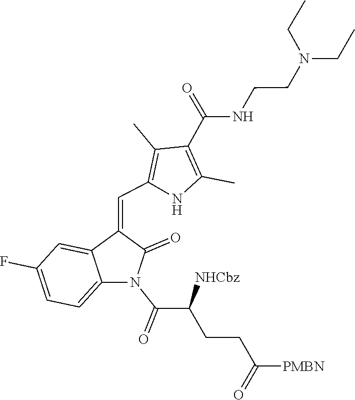 |
| 52 | 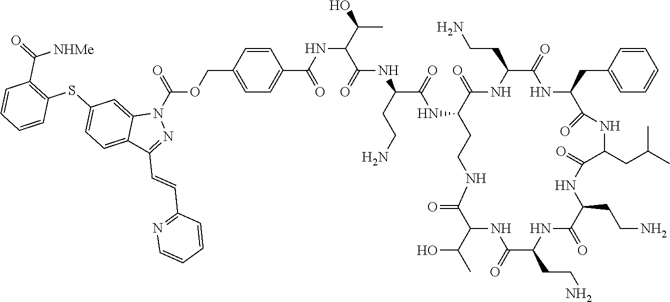 [SMILES: CNC(=O)c1ccccc1Sc1ccc2c(/C=C/c3ccccn3)nn(C(=O)OCc3ccc(C(=O)NC(C(=O)N[C@H](CCN)C(=O)N[C@H]4CCNC(=O)C(C(C)O)NC(=O)[C@H](CCN)NC(=O)[C@H](CCN)NC(=O)C(CC(C)C)NC(=O)[C@H](Cc5ccccc5)NC(=O)[C@H](CCN)NC4=O)[C@H](C)O)cc3)c2c1] |
| 53 | 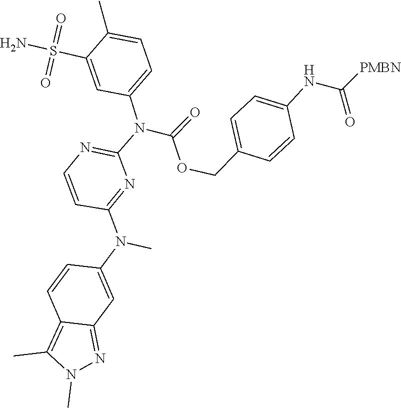 [SMILES: Cc1ccc(N(C(=O)OCc2ccc(NC=O)cc2)c2nccc(N(C)c3ccc4c(C)n(C)nc4c3)n2)cc1S(N)(=O)=O ; "PMBN"] |
| 54 | 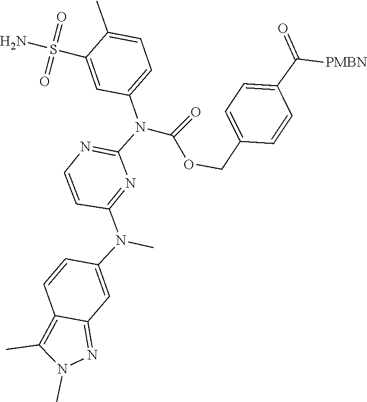 |
| 55 | 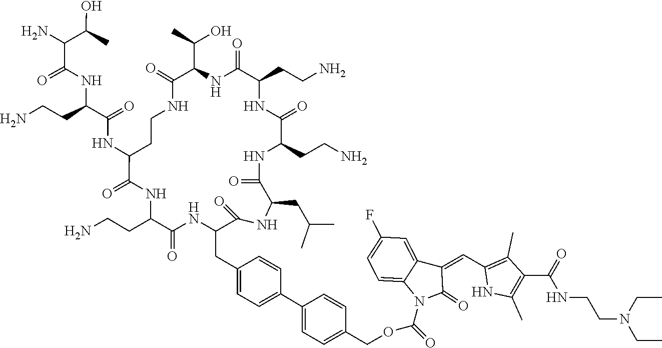 [SMILES: CCN(CC)CCNC(=O)c1c(C)[nH]c(/C=C2\C(=O)N(C(=O)OCc3ccc(-c4ccc(CC5NC(=O)C(CCN)NC(=O)C(NC(=O)[C@@H](CCN)NC(=O)C(N)[C@H](C)O)CCNC(=O)[C@@H]([C@@H](C)O)NC(=O)[C@@H](CCN)NC(=O)[C@@H](CCN)NC(=O)[C@@H](CC(C)C)NC5=O)cc4)cc3)c3ccc(F)cc32)c1C] |
| 56 | 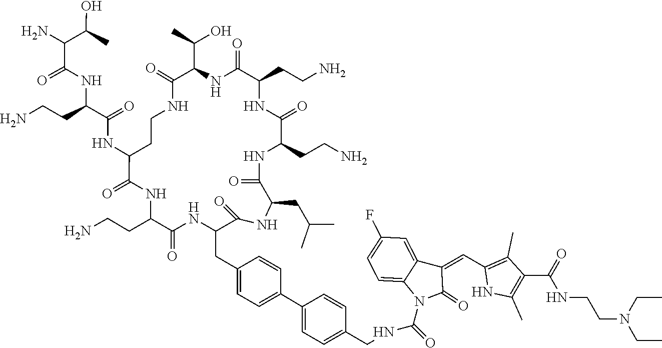 [SMILES: CCN(CC)CCNC(=O)c1c(C)[nH]c(/C=C2\C(=O)N(C(=O)NCc3ccc(-c4ccc(CC5NC(=O)C(CCN)NC(=O)C(NC(=O)[C@@H](CCN)NC(=O)C(N)[C@H](C)O)CCNC(=O)[C@@H]([C@@H](C)O)NC(=O)[C@@H](CCN)NC(=O)[C@@H](CCN)NC(=O)[C@@H](CC(C)C)NC5=O)cc4)cc3)c3ccc(F)cc32)c1C] |
| 57 | 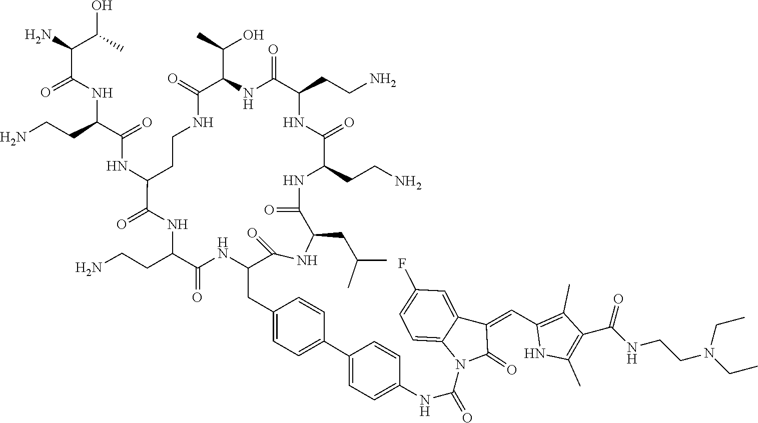 [SMILES: CCN(CC)CCNC(=O)c1c(C)[nH]c(/C=C2\C(=O)N(C(=O)Nc3ccc(-c4ccc(CC5NC(=O)C(CCN)NC(=O)C(NC(=O)[C@@H](CCN)NC(=O)[C@@H](N)[C@@H](C)O)CCNC(=O)[C@@H]([C@@H](C)O)NC(=O)[C@@H](CCN)NC(=O)[C@@H](CCN)NC(=O)[C@@H](CC(C)C)NC5=O)cc4)cc3)c3ccc(F)cc32)c1C] |
| 64 | 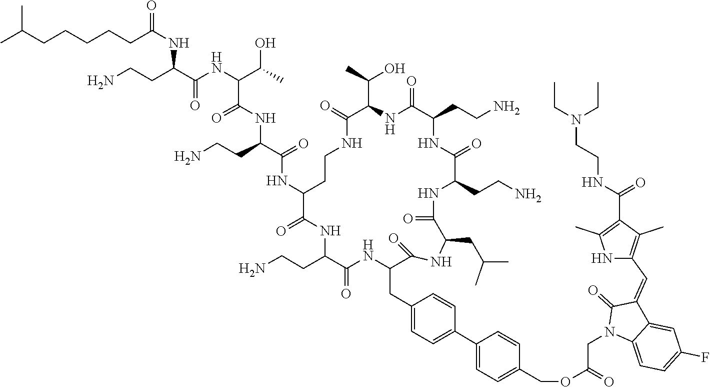 [SMILES: CCN(CC)CCNC(=O)c1c(C)[nH]c(/C=C2\C(=O)N(CC(=O)OCc3ccc(-c4ccc(CC5NC(=O)C(CCN)NC(=O)C(NC(=O)[C@@H](CCN)NC(=O)C(NC(=O)[C@@H](CCN)NC(=O)CCCCCC(C)C)[C@@H](C)O)CCNC(=O)[C@@H]([C@@H](C)O)NC(=O)[C@@H](CCN)NC(=O)[C@@H](CCN)NC(=O)[C@@H](CC(C)C)NC5=O)cc4)cc3)c3ccc(F)cc32)c1C] |
| 65 | 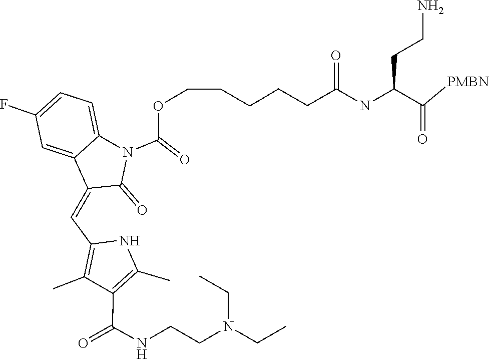 |
| 66 | 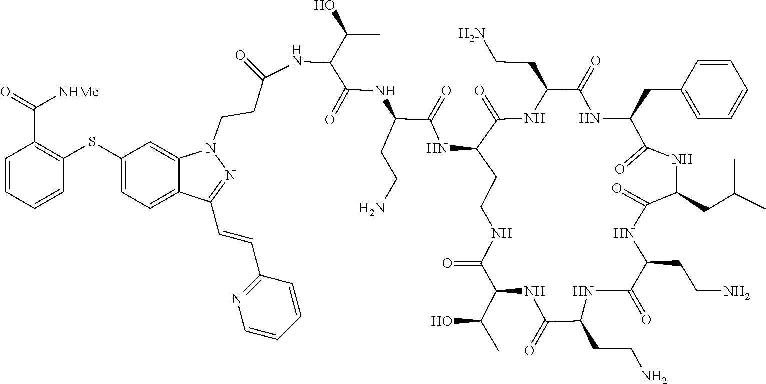 [SMILES: CNC(=O)c1ccccc1Sc1ccc2c(/C=C/c3ccccn3)nn(CCC(=O)NC(C(=O)N[C@H](CCN)C(=O)N[C@@H]3CCNC(=O)[C@H]([C@@H](C)O)NC(=O)[C@H](CCN)NC(=O)[C@H](CCN)NC(=O)[C@H](CC(C)C)NC(=O)[C@H](Cc4ccccc4)NC(=O)[C@H](CCN)NC3=O)[C@H](C)O)c2c1] |
| 67 | 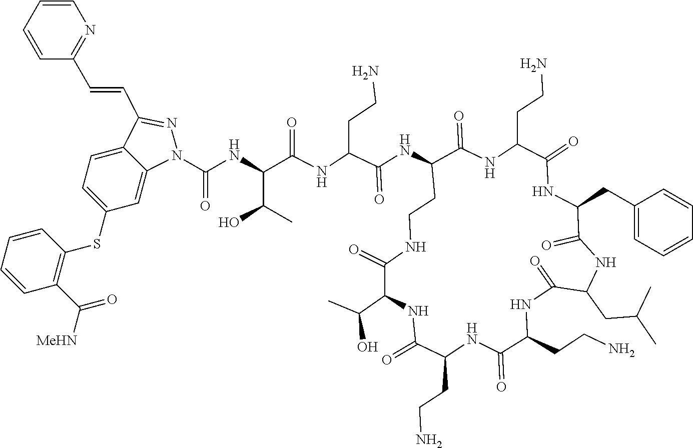 [SMILES: CNC(=O)c1ccccc1Sc1ccc2c(/C=C/c3ccccn3)nn(C(=O)N[C@@H](C(=O)NC(CCN)C(=O)N[C@@H]3CCNC(=O)[C@H]([C@H](C)O)NC(=O)[C@H](CCN)NC(=O)[C@H](CCN)NC(=O)C(CC(C)C)NC(=O)[C@H](Cc4ccccc4)NC(=O)C(CCN)NC3=O)[C@@H](C)O)c2c1] |
| 68 | 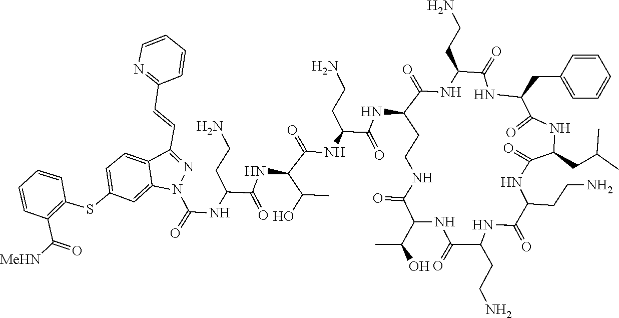 [SMILES: CNC(=O)c1ccccc1Sc1ccc2c(/C=C/c3ccccn3)nn(C(=O)NC(CCN)C(=O)N[C@@H](C(=O)N[C@@H](CCN)C(=O)N[C@@H]3CCNC(=O)C([C@H](C)O)NC(=O)C(CCN)NC(=O)C(CCN)NC(=O)[C@H](CC(C)C)NC(=O)[C@H](Cc4ccccc4)NC(=O)[C@H](CCN)NC3=O)C(C)O)c2c1] |
| 69 | 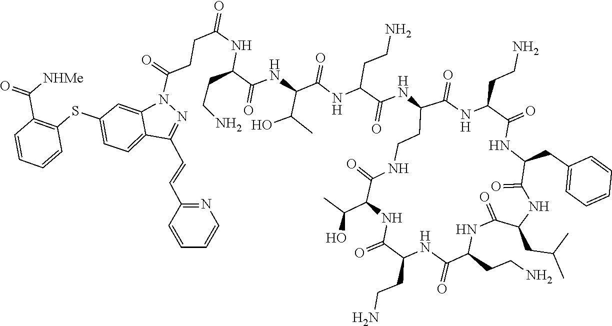 [SMILES: CNC(=O)c1ccccc1Sc1ccc2c(/C=C/c3ccccn3)nn(C(=O)CCC(=O)N[C@H](CCN)C(=O)N[C@@H](C(=O)NC(CCN)C(=O)N[C@@H]3CCNC(=O)[C@H]([C@H](C)O)NC(=O)[C@H](CCN)NC(=O)[C@H](CCN)NC(=O)[C@H](CC(C)C)NC(=O)[C@H](Cc4ccccc4)NC(=O)[C@H](CCN)NC3=O)C(C)O)c2c1] |
| 76 | 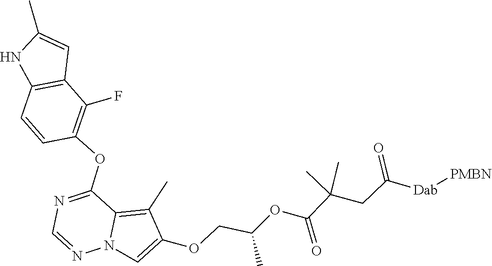 |
| 77 | 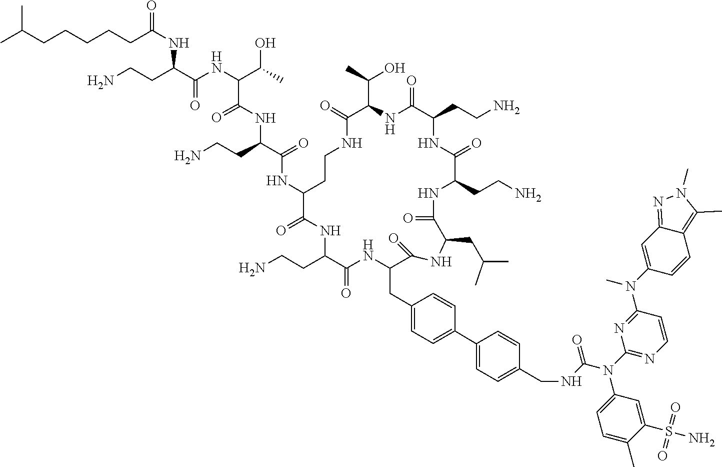 [SMILES: Cc1ccc(N(C(=O)NCc2ccc(-c3ccc(CC4NC(=O)C(CCN)NC(=O)C(NC(=O)[C@@H](CCN)NC(=O)C(NC(=O)[C@@H](CCN)NC(=O)CCCCCC(C)C)[C@@H](C)O)CCNC(=O)[C@@H]([C@@H](C)O)NC(=O)[C@@H](CCN)NC(=O)[C@@H](CCN)NC(=O)[C@@H](CC(C)C)NC4=O)cc3)cc2)c2nccc(N(C)c3ccc4c(C)n(C)nc4c3)n2)cc1S(N)(=O)=O] |
| 78 | 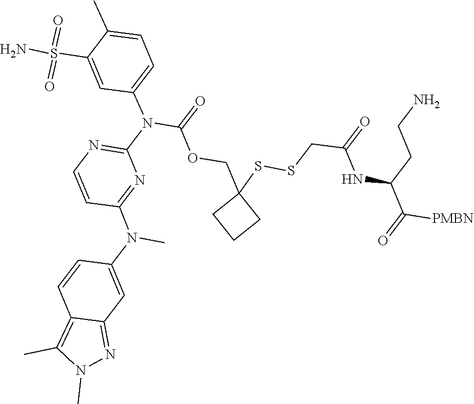 |
| 79 | 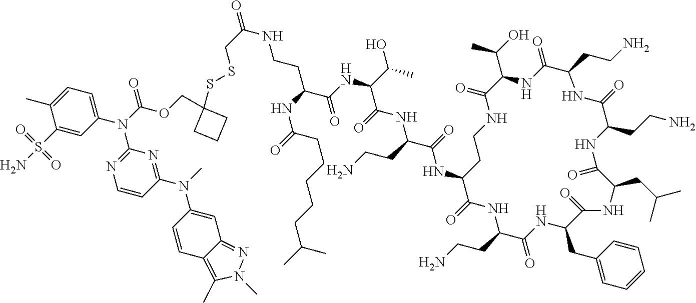 [SMILES: Cc1ccc(N(C(=O)OCC2(SSCC(=O)NCC[C@H](NC(=O)CCCCCC(C)C)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)CCC2)c2nccc(N(C)c3ccc4c(C)n(C)nc4c3)n2)cc1S(N)(=O)=O] |
| 80 | 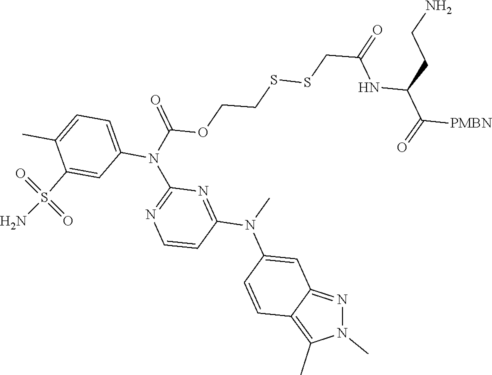 |
| 81 | 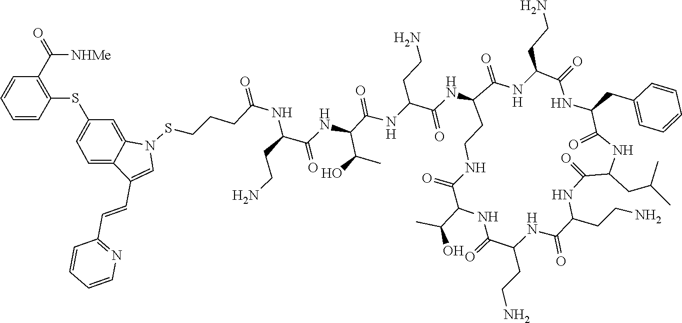 [SMILES: CNC(=O)c1ccccc1Sc1ccc2c(/C=C/c3ccccn3)cn(SCCCC(=O)N[C@H](CCN)C(=O)N[C@@H](C(=O)NC(CCN)C(=O)N[C@@H]3CCNC(=O)C([C@H](C)O)NC(=O)C(CCN)NC(=O)C(CCN)NC(=O)C(CC(C)C)NC(=O)[C@H](Cc4ccccc4)NC(=O)[C@H](CCN)NC3=O)[C@@H](C)O)c2c1] |
| 88 | 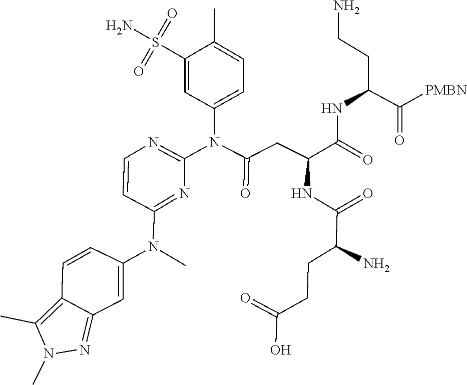 |
| 89 | 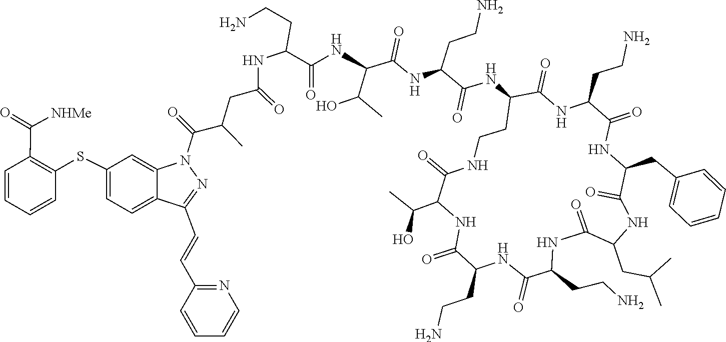 [SMILES: CNC(=O)c1ccccc1Sc1ccc2c(/C=C/c3ccccn3)nn(C(=O)C(C)CC(=O)NC(CCN)C(=O)N[C@@H](C(=O)N[C@@H](CCN)C(=O)N[C@@H]3CCNC(=O)C([C@H](C)O)NC(=O)[C@H](CCN)NC(=O)[C@H](CCN)NC(=O)C(CC(C)C)NC(=O)[C@H](Cc4ccccc4)NC(=O)[C@H](CCN)NC3=O)C(C)O)c2c1] |
| 90 | 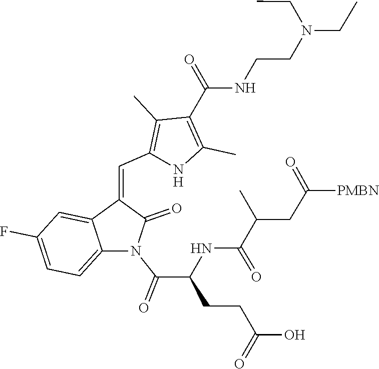 |
| 91 | 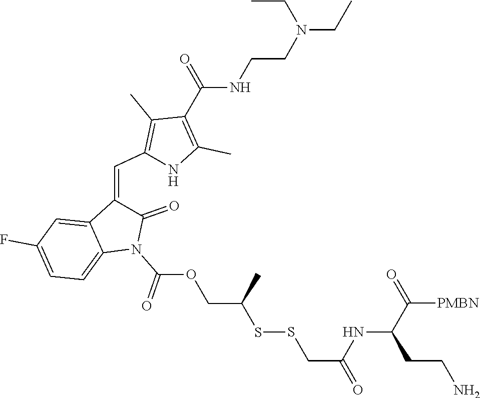 |
| 92 | 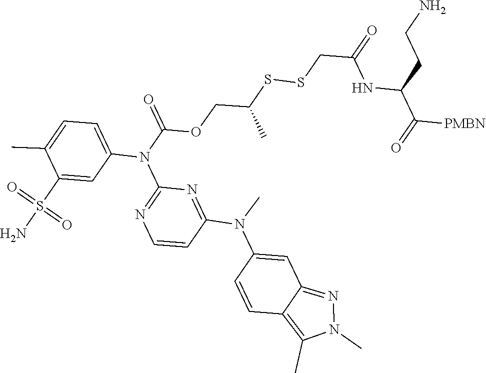 |
| 93 | 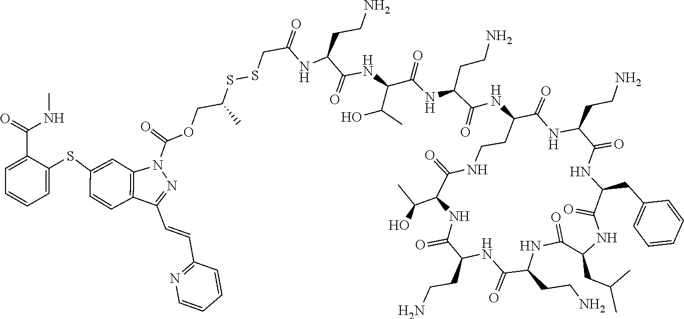 |
| 100 | 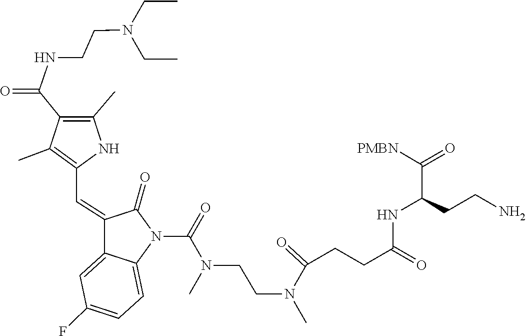 |
| 101 | 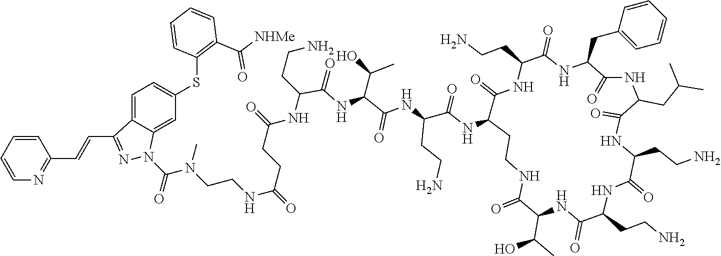 [SMILES: CNC(=O)c1ccccc1Sc1ccc2c(/C=C/c3ccccn3)nn(C(=O)N(C)CCNC(=O)CCC(=O)NC(CCN)C(=O)N[C@H](C(=O)N[C@H](CCN)C(=O)N[C@@H]3CCNC(=O)[C@H]([C@@H](C)O)NC(=O)[C@H](CCN)NC(=O)[C@H](CCN)NC(=O)C(CC(C)C)NC(=O)[C@H](Cc4ccccc4)NC(=O)[C@H](CCN)NC3=O)[C@H](C)O)c2c1] |
| 102 | 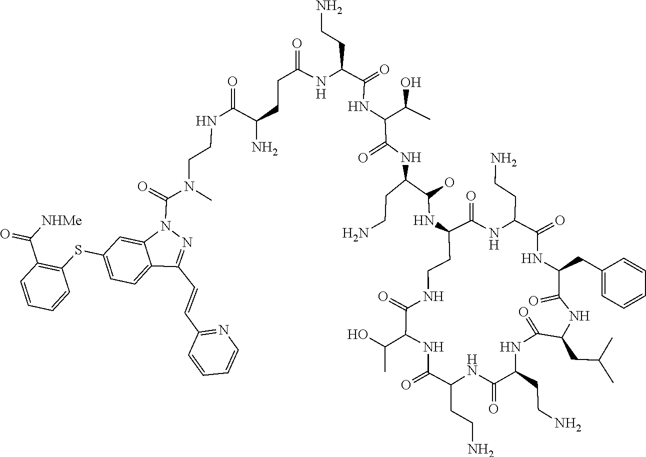 |
| 103 | 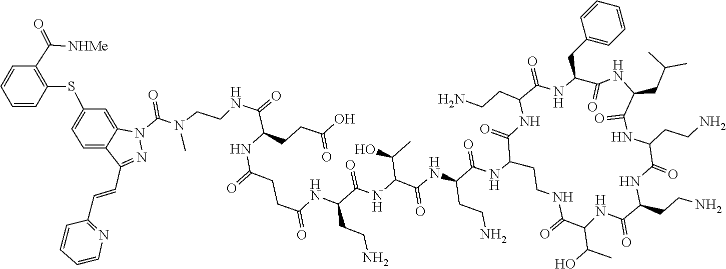 [SMILES: CNC(=O)c1ccccc1Sc1ccc2c(/C=C/c3ccccn3)nn(C(=O)N(C)CCNC(=O)[C@@H](CCC(=O)O)NC(=O)CCC(=O)N[C@H](CCN)C(=O)NC(C(=O)N[C@H](CCN)C(=O)NC3CCNC(=O)C(C(C)O)NC(=O)[C@H](CCN)NC(=O)C(CCN)NC(=O)[C@H](CC(C)C)NC(=O)[C@H](Cc4ccccc4)NC(=O)C(CCN)NC3=O)[C@H](C)O)c2c1] |
| 104 | 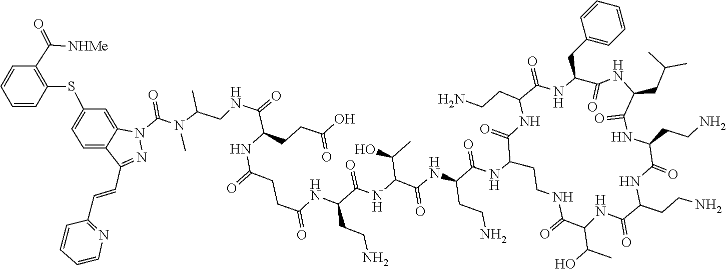 [SMILES: CNC(=O)c1ccccc1Sc1ccc2c(/C=C/c3ccccn3)nn(C(=O)N(C)C(C)CNC(=O)[C@@H](CCC(=O)O)NC(=O)CCC(=O)N[C@H](CCN)C(=O)NC(C(=O)N[C@H](CCN)C(=O)NC3CCNC(=O)C(C(C)O)NC(=O)C(CCN)NC(=O)[C@H](CCN)NC(=O)[C@H](CC(C)C)NC(=O)[C@H](Cc4ccccc4)NC(=O)C(CCN)NC3=O)[C@H](C)O)c2c1] |
| 105 | 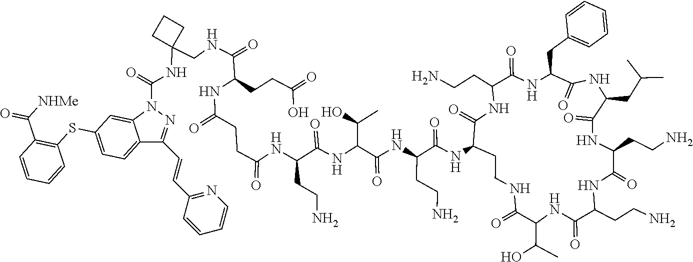 [SMILES: CNC(=O)c1ccccc1Sc1ccc2c(/C=C/c3ccccn3)nn(C(=O)NC3(CNC(=O)[C@@H](CCC(=O)O)NC(=O)CCC(=O)N[C@H](CCN)C(=O)NC(C(=O)N[C@H](CCN)C(=O)N[C@@H]4CCNC(=O)C(C(C)O)NC(=O)C(CCN)NC(=O)[C@H](CCN)NC(=O)[C@H](CC(C)C)NC(=O)[C@H](Cc5ccccc5)NC(=O)C(CCN)NC4=O)[C@H](C)O)CCC3)c2c1] |
| 112 | 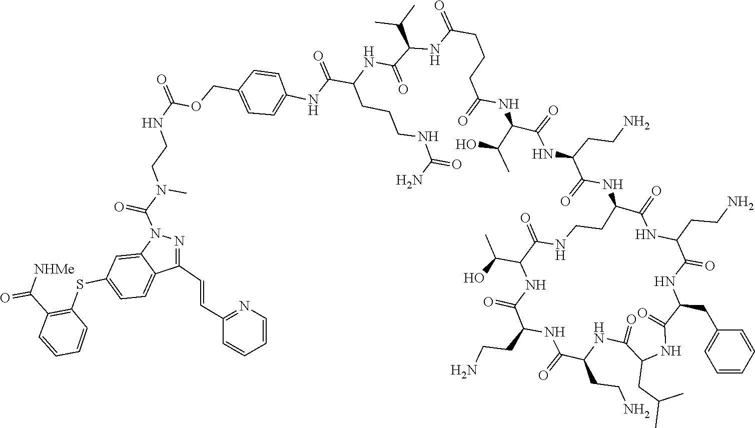 [SMILES: CNC(=O)c1ccccc1Sc1ccc2c(/C=C/c3ccccn3)nn(C(=O)N(C)CCNC(=O)OCc3ccc(NC(=O)C(CCCNC(N)=O)NC(=O)[C@H](NC(=O)CCCC(=O)N[C@@H](C(=O)N[C@@H](CCN)C(=O)N[C@@H]4CCNC(=O)C([C@H](C)O)NC(=O)[C@H](CCN)NC(=O)[C@H](CCN)NC(=O)C(CC(C)C)NC(=O)[C@H](Cc5ccccc5)NC(=O)C(CCN)NC4=O)[C@@H](C)O)C(C)C)cc3)c2c1] |
| 113 | 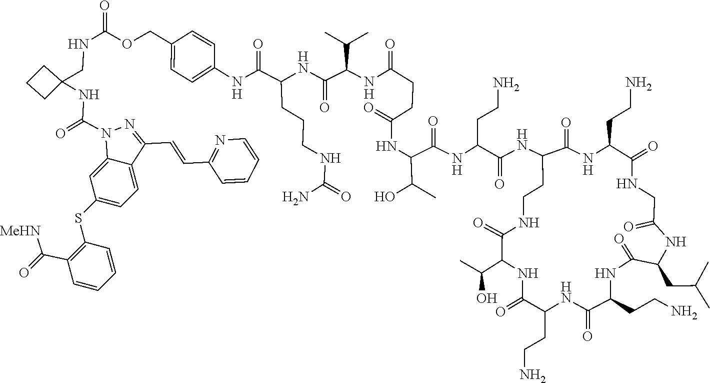 [SMILES: CNC(=O)c1ccccc1Sc1ccc2c(/C=C/c3ccccn3)nn(C(=O)NC3(CNC(=O)OCc4ccc(NC(=O)C(CCCNC(N)=O)NC(=O)[C@H](NC(=O)CCC(=O)NC(C(=O)NC(CCN)C(=O)NC5CCNC(=O)C([C@H](C)O)NC(=O)C(CCN)NC(=O)[C@H](CCN)NC(=O)[C@H](CC(C)C)NC(=O)CNC(=O)[C@H](CCN)NC5=O)C(C)O)C(C)C)cc4)CCC3)c2c1] |
| 114 | 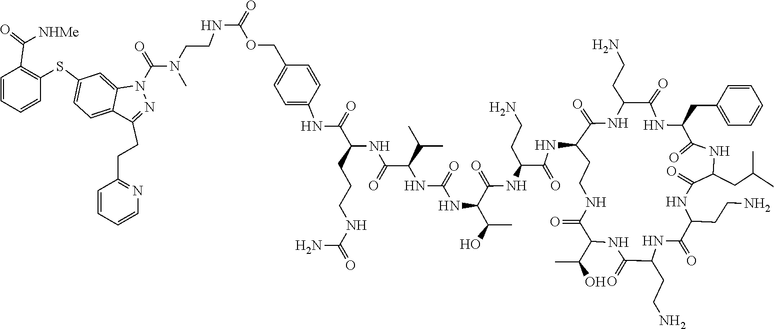 [SMILES: CNC(=O)c1ccccc1Sc1ccc2c(CCc3ccccn3)nn(C(=O)N(C)CCNC(=O)OCc3ccc(NC(=O)[C@H](CCCNC(N)=O)NC(=O)[C@H](NC(=O)N[C@@H](C(=O)N[C@@H](CCN)C(=O)N[C@@H]4CCNC(=O)C([C@H](C)O)NC(=O)C(CCN)NC(=O)C(CCN)NC(=O)C(CC(C)C)NC(=O)[C@H](Cc5ccccc5)NC(=O)C(CCN)NC4=O)[C@@H](C)O)C(C)C)cc3)c2c1] |
| 115 | 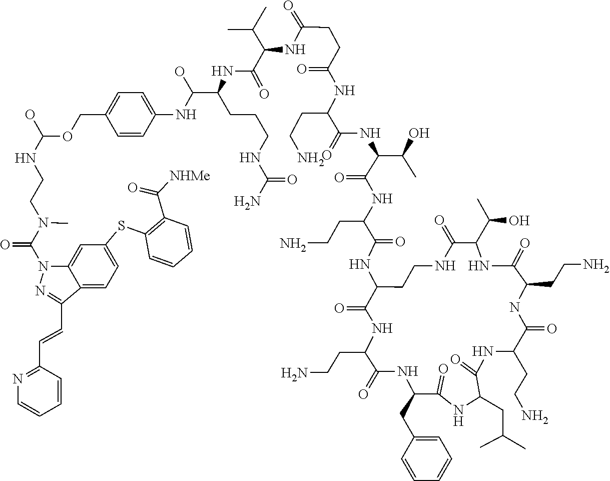 |
| 116 | 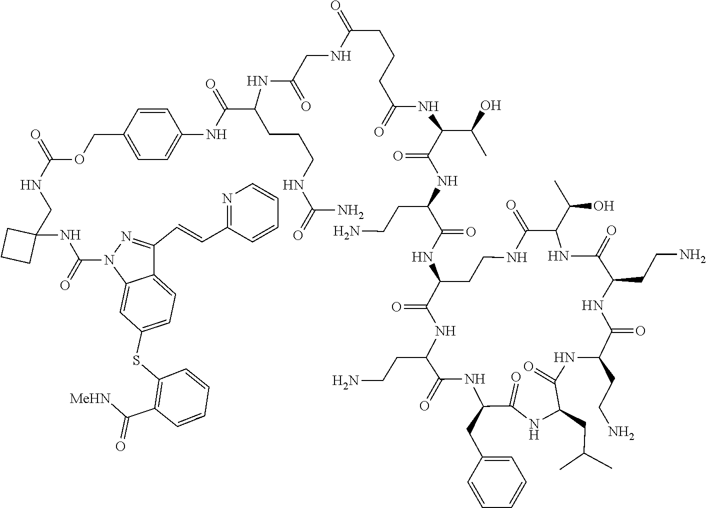 |
| 117 | 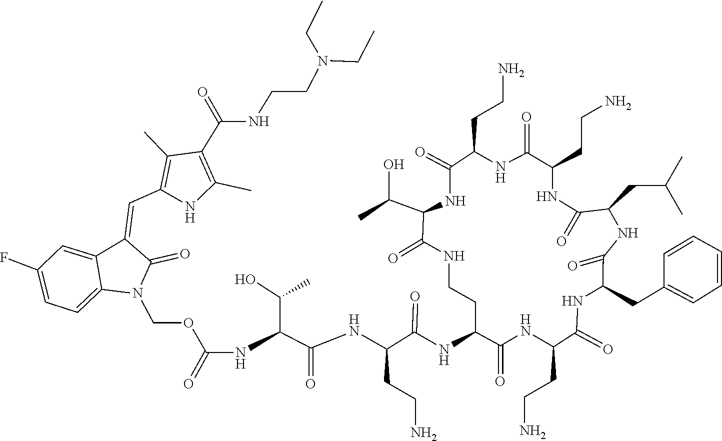 [SMILES: CCN(CC)CCNC(=O)c1c(C)[nH]c(/C=C2\C(=O)N(COC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 124 | 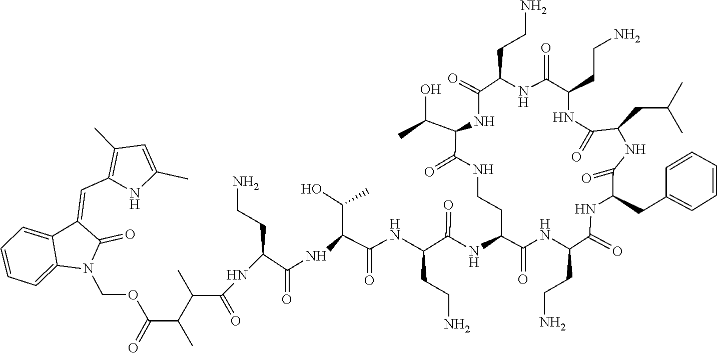 [SMILES: Cc1cc(C)c(/C=C2\C(=O)N(COC(=O)C(C)C(C)C(=O)N[C@@H](CCN)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccccc32)[nH]1] |
| 125 | 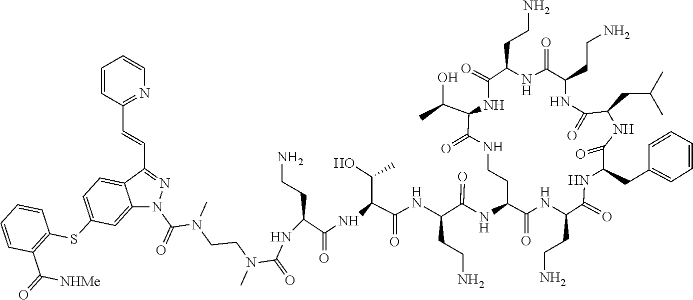 [SMILES: CNC(=O)c1ccccc1Sc1ccc2c(/C=C/c3ccccn3)nn(C(=O)N(C)CCN(C)C(=O)N[C@@H](CCN)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 126 | 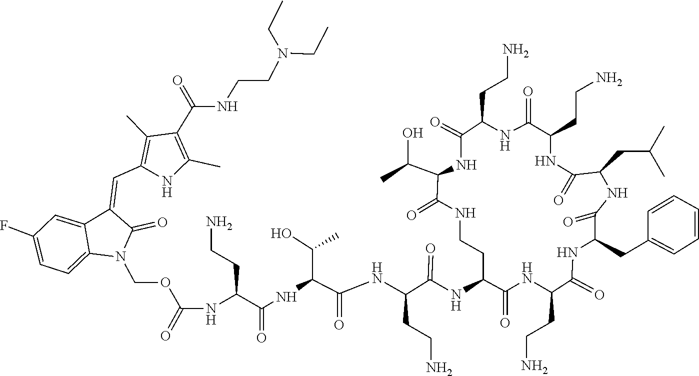 [SMILES: CCN(CC)CCNC(=O)c1c(C)[nH]c(/C=C2\C(=O)N(COC(=O)N[C@@H](CCN)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 127 | 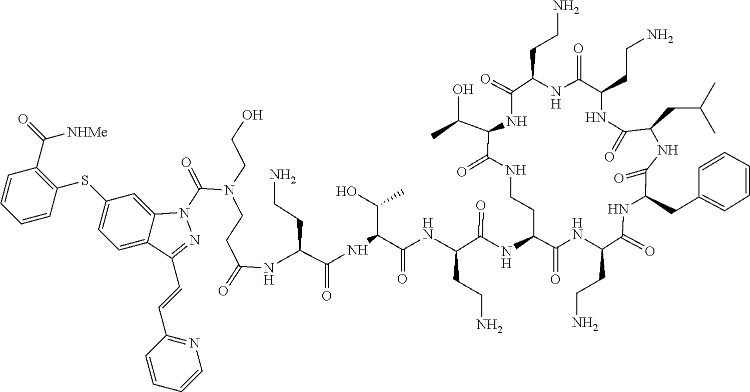 [SMILES: CNC(=O)c1ccccc1Sc1ccc2c(/C=C/c3ccccn3)nn(C(=O)N(CCO)CCC(=O)N[C@@H](CCN)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 128 | 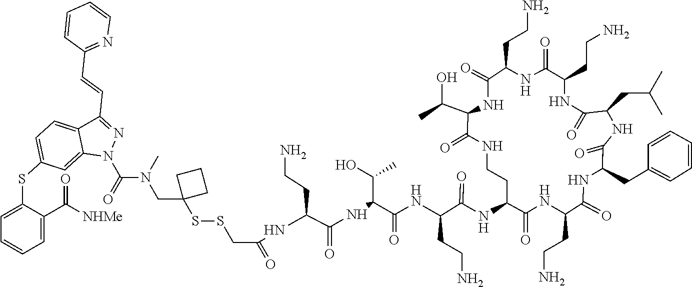 [SMILES: CNC(=O)c1ccccc1Sc1ccc2c(/C=C/c3ccccn3)nn(C(=O)N(C)CC3(SSCC(=O)N[C@@H](CCN)C(=O)N[C@H](C(=O)N[C@H](CCN)C(=O)N[C@H]4CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc5ccccc5)NC(=O)[C@@H](CCN)NC4=O)[C@@H](C)O)CCC3)c2c1] |
| 129 | 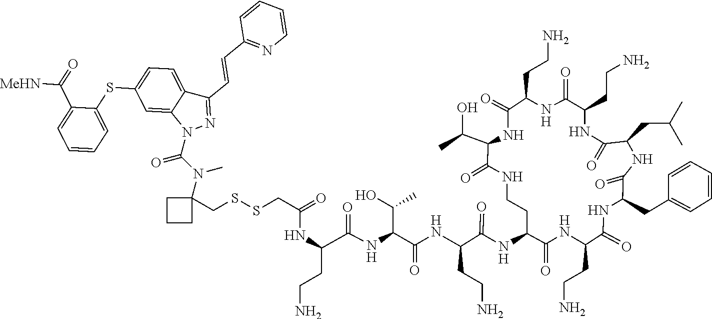 [SMILES: CNC(=O)c1ccccc1Sc1ccc2c(/C=C/c3ccccn3)nn(C(=O)N(C)C3(CSSCC(=O)N[C@H](CCN)C(=O)N[C@H](C(=O)N[C@H](CCN)C(=O)N[C@H]4CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc5ccccc5)NC(=O)[C@@H](CCN)NC4=O)[C@@H](C)O)CCC3)c2c1] |
| 134 | 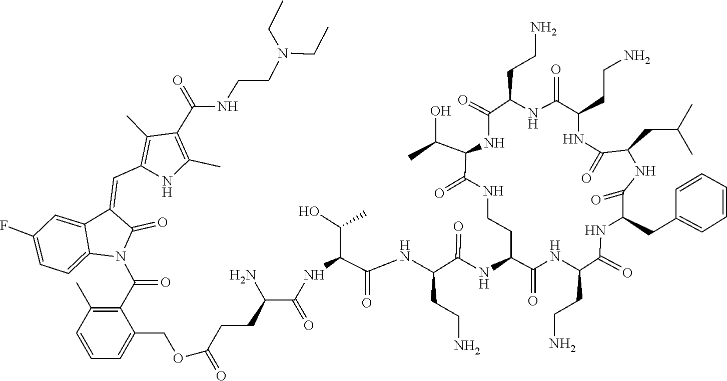 [SMILES: CCN(CC)CCNC(=O)c1c(C)[nH]c(/C=C2\C(=O)N(C(=O)c3c(C)cccc3COC(=O)CC[C@@H](N)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 135 | 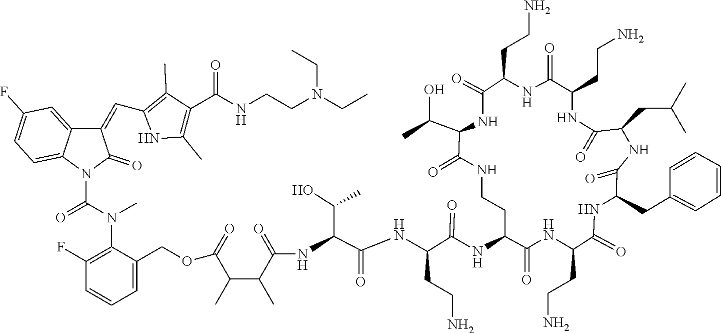 [SMILES: CCN(CC)CCNC(=O)c1c(C)[nH]c(/C=C2\C(=O)N(C(=O)N(C)c3c(F)cccc3COC(=O)C(C)C(C)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 136 | 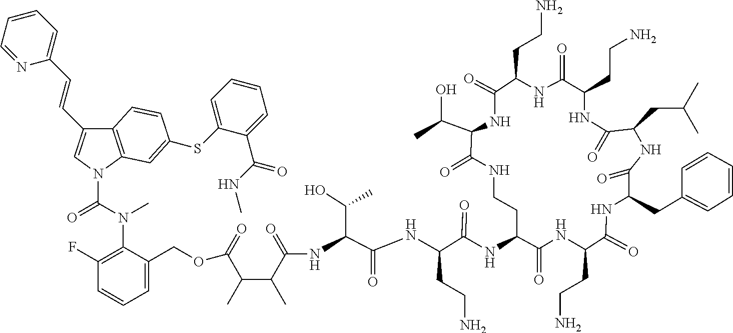 [SMILES: CNC(=O)c1ccccc1Sc1ccc2c(/C=C/c3ccccn3)cn(C(=O)N(C)c3c(F)cccc3COC(=O)C(C)C(C)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 137 | 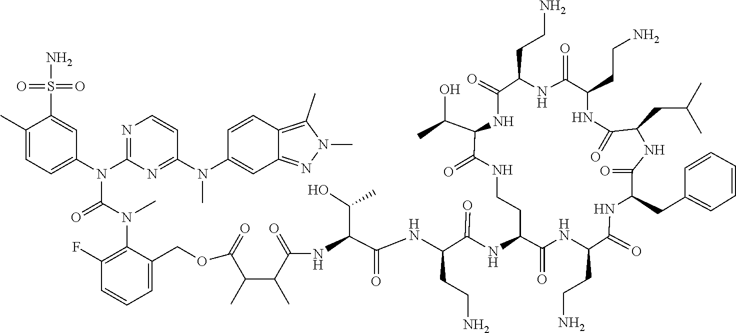 [SMILES: Cc1ccc(N(C(=O)N(C)c2c(F)cccc2COC(=O)C(C)C(C)C(=O)N[C@H](C(=O)N[C@H](CCN)C(=O)N[C@H]2CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc3ccccc3)NC(=O)[C@@H](CCN)NC2=O)[C@@H](C)O)c2nccc(N(C)c3ccc4c(C)n(C)nc4c3)n2)cc1S(N)(=O)=O] |
| 138 | 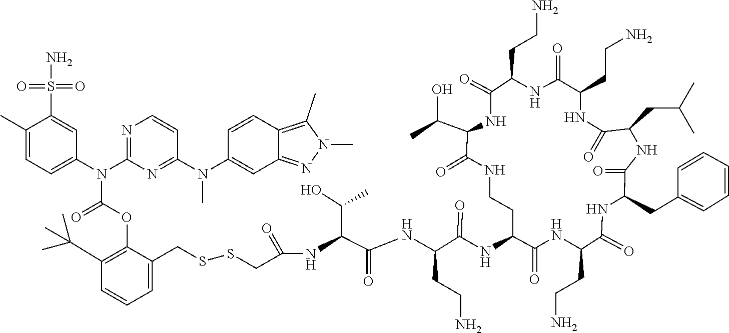 [SMILES: Cc1ccc(N(C(=O)Oc2c(CSSCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)cccc2C(C)(C)C)c2nccc(N(C)c3ccc4c(C)n(C)nc4c3)n2)cc1S(N)(=O)=O] |
| 139 | 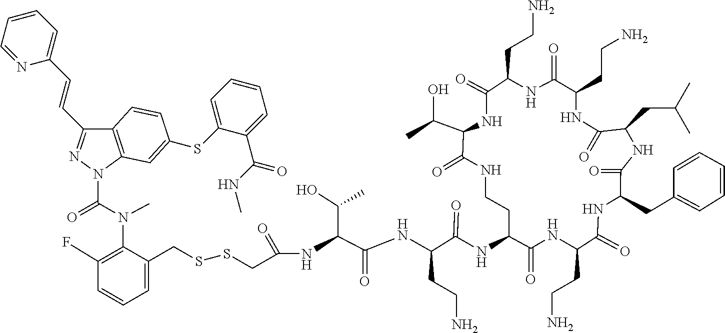 [SMILES: CNC(=O)c1ccccc1Sc1ccc2c(/C=C/c3ccccn3)nn(C(=O)N(C)c3c(F)cccc3CSSCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 146 | 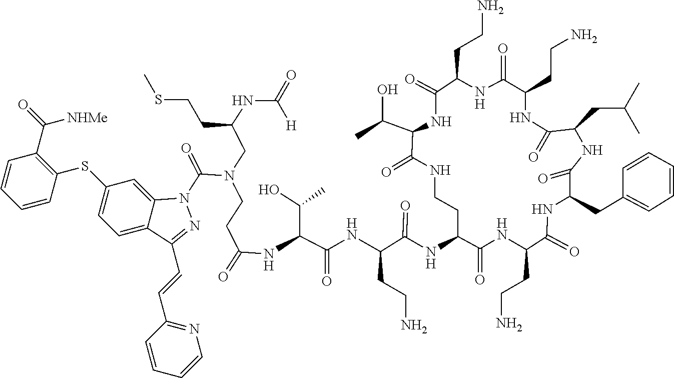 [SMILES: CNC(=O)c1ccccc1Sc1ccc2c(/C=C/c3ccccn3)nn(C(=O)N(CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)C[C@@H](CCSC)NC=O)c2c1] |
| 147 | 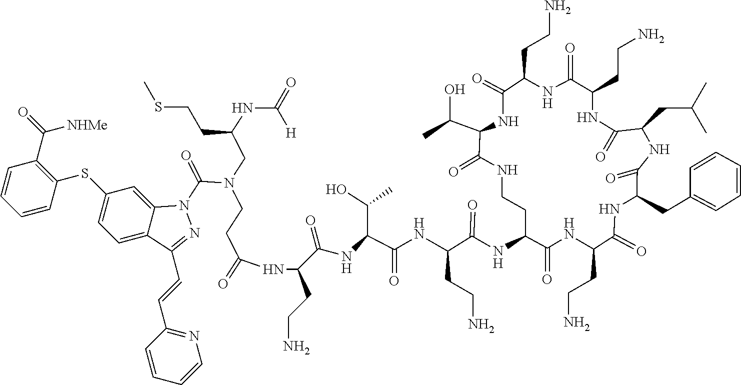 [SMILES: CNC(=O)c1ccccc1Sc1ccc2c(/C=C/c3ccccn3)nn(C(=O)N(CCC(=O)N[C@H](CCN)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)C[C@@H](CCSC)NC=O)c2c1] |
| 150 | 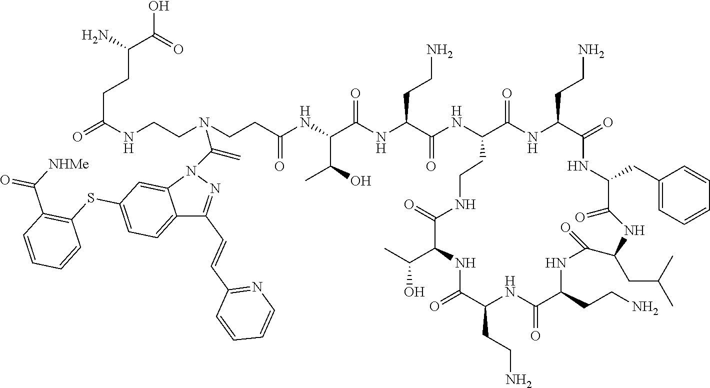 [SMILES: C=C(N(CCNC(=O)CC[C@H](N)C(=O)O)CCC(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@H](C)O)n1nc(/C=C/c2ccccn2)c2ccc(Sc3ccccc3C(=O)NC)cc21] |
| 151 | 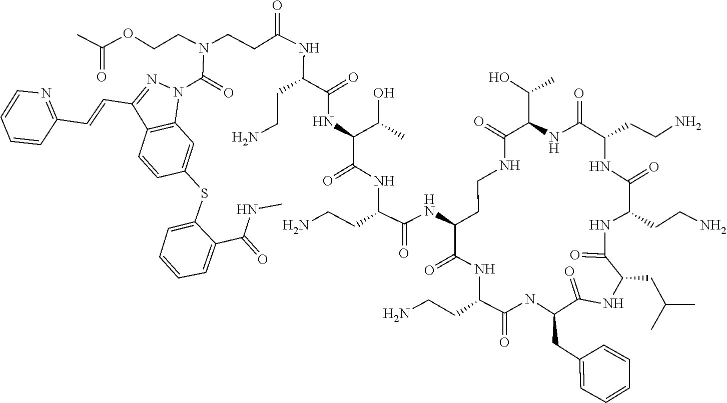 |
| 152 | 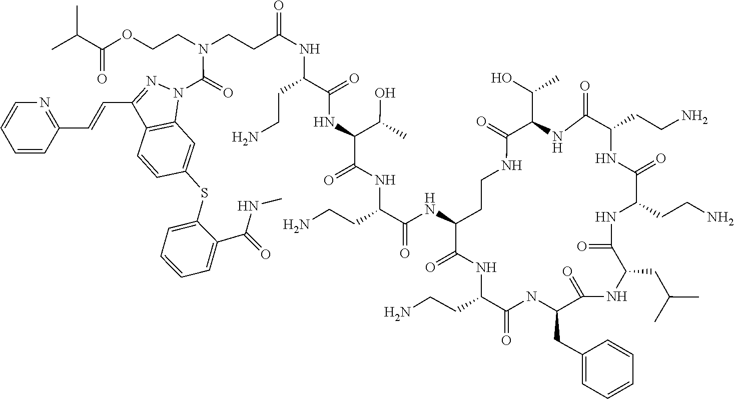 |
| 153 | 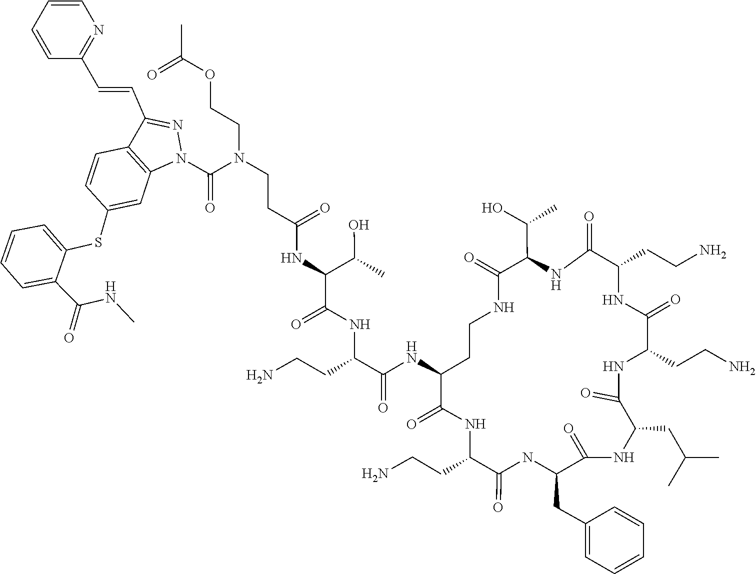 |
| 158 | 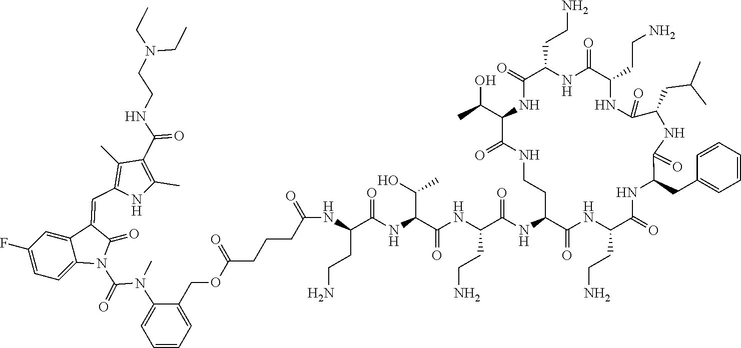 [SMILES: CCN(CC)CCNC(=O)c1c(C)[nH]c(/C=C2\C(=O)N(C(=O)N(C)c3ccccc3COC(=O)CCCC(=O)N[C@H](CCN)C(=O)N[C@H](C(=O)N[C@@H](CCN)C(=O)N[C@H]3CCNC(=O)[C@@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 159 | 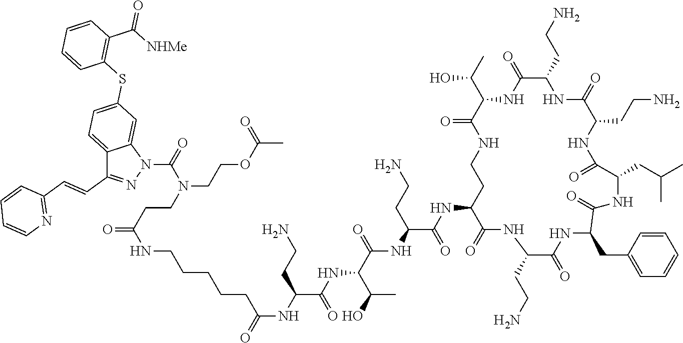 [SMILES: CNC(=O)c1ccccc1Sc1ccc2c(/C=C/c3ccccn3)nn(C(=O)N(CCOC(C)=O)CCC(=O)NCCCCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 160 | 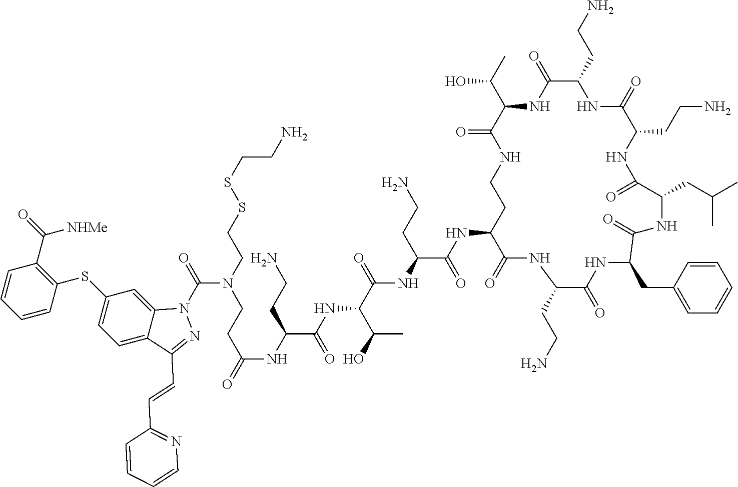 [SMILES: CNC(=O)c1ccccc1Sc1ccc2c(/C=C/c3ccccn3)nn(C(=O)N(CCSSCCN)CCC(=O)N[C@@H](CCN)C(=O)N[C@H](C(=O)N[C@@H](CCN)C(=O)N[C@H]3CCNC(=O)[C@@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 161 | 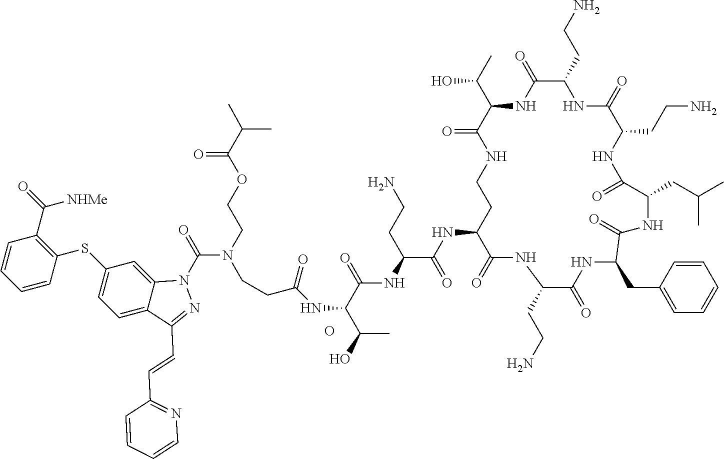 |
| 162 | 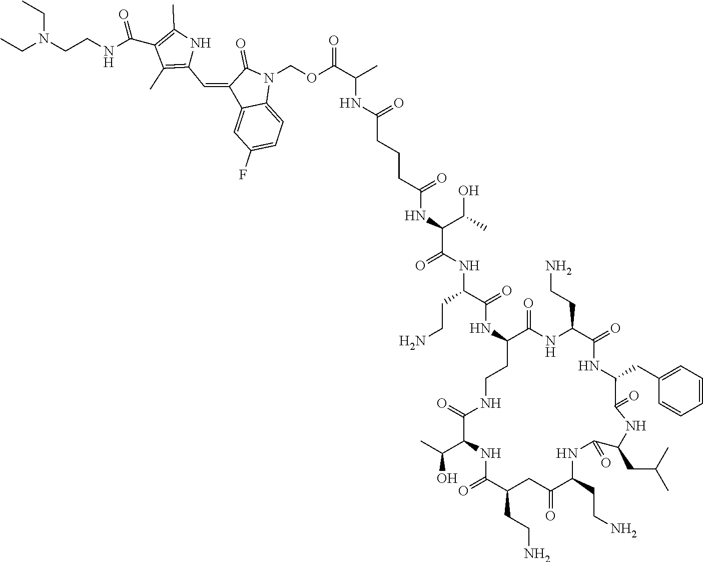 [SMILES: CCN(CC)CCNC(=O)c1c(C)[nH]c(/C=C2\C(=O)N(COC(=O)C(C)NC(=O)CCCC(=O)N[C@H](C(=O)N[C@@H](CCN)C(=O)N[C@@H]3CCNC(=O)[C@H]([C@H](C)O)NC(=O)[C@H](CCN)CC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 163 | 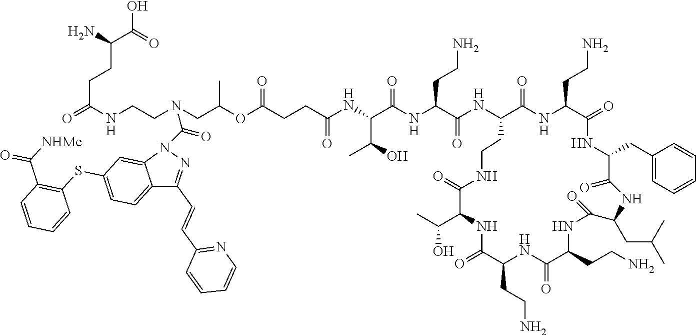 [SMILES: CNC(=O)c1ccccc1Sc1ccc2c(/C=C/c3ccccn3)nn(C(=O)N(CCNC(=O)CC[C@@H](N)C(=O)O)CC(C)OC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 170 | 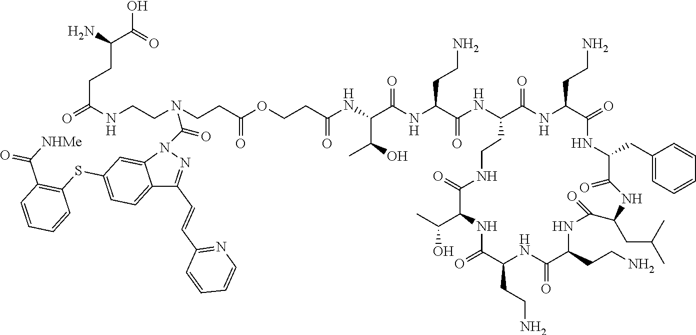 [SMILES: CNC(=O)c1ccccc1Sc1ccc2c(/C=C/c3ccccn3)nn(C(=O)N(CCNC(=O)CC[C@@H](N)C(=O)O)CCC(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 172 | 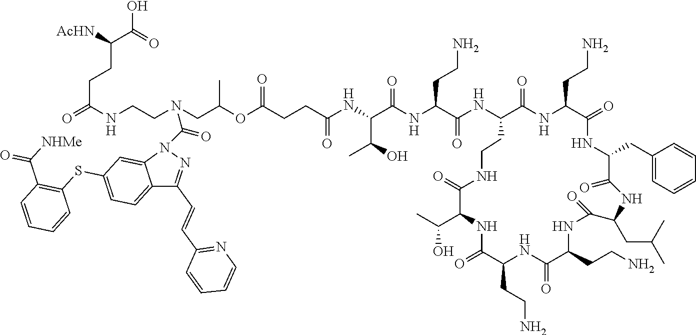 [SMILES: CNC(=O)c1ccccc1Sc1ccc2c(/C=C/c3ccccn3)nn(C(=O)N(CCNC(=O)CC[C@@H](NC(C)=O)C(=O)O)CC(C)OC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 173 | 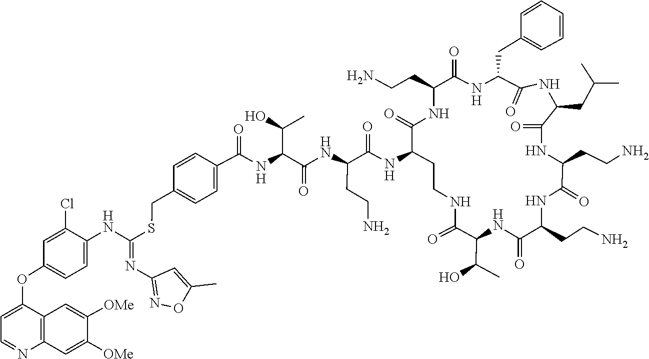 [SMILES: COc1cc2nccc(Oc3ccc(N/C(=N/c4cc(C)on4)SCc4ccc(C(=O)N[C@H](C(=O)N[C@H](CCN)C(=O)N[C@@H]5CCNC(=O)[C@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@H](C)O)cc4)c(Cl)c3)c2cc1OC] |
| Cpd. No. | Structure |
| 8 | 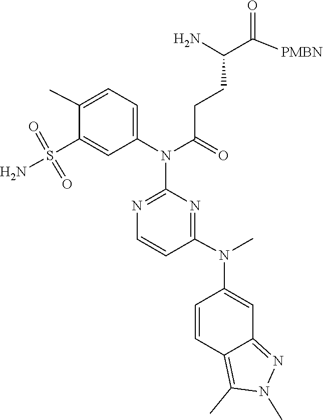 [SMILES: Cc1ccc(N(C(=O)CC[C@H](N)C=O)c2nccc(N(C)c3ccc4c(C)n(C)nc4c3)n2)cc1S(N)(=O)=O ; "PMBN"] |
| 9 | 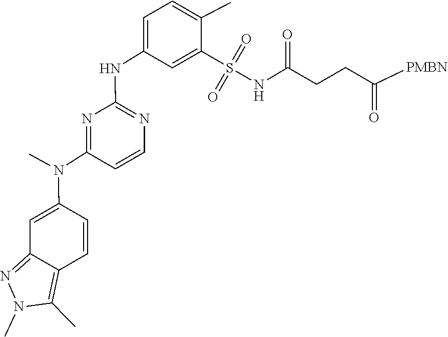 |
| 10 | 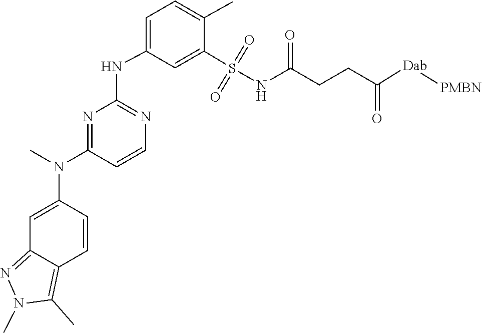 |
| 11 | 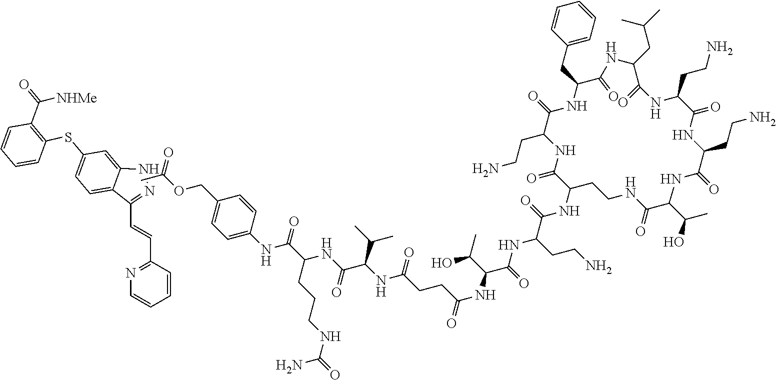 |
| 12 | 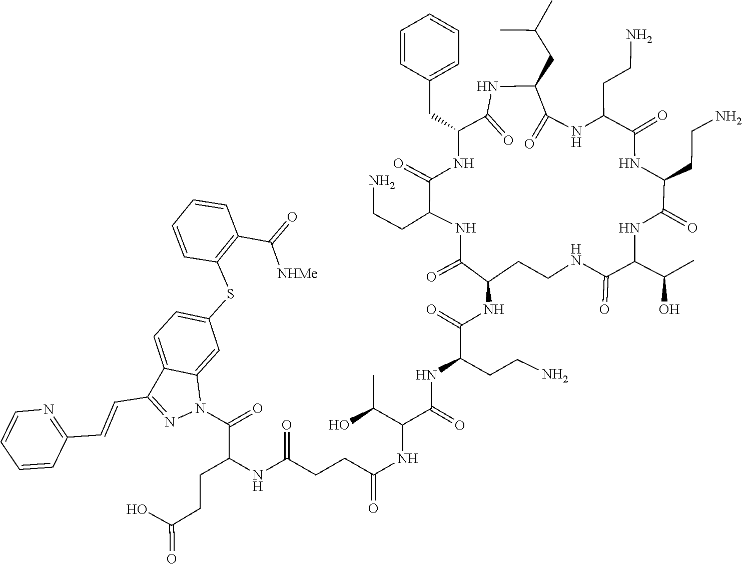 [SMILES: CNC(=O)c1ccccc1Sc1ccc2c(/C=C/c3ccccn3)nn(C(=O)C(CCC(=O)O)NC(=O)CCC(=O)NC(C(=O)N[C@H](CCN)C(=O)N[C@@H]3CCNC(=O)C([C@@H](C)O)NC(=O)[C@H](CCN)NC(=O)C(CCN)NC(=O)[C@H](CC(C)C)NC(=O)[C@@H](Cc4ccccc4)NC(=O)C(CCN)NC3=O)[C@H](C)O)c2c1] |
| 13 | 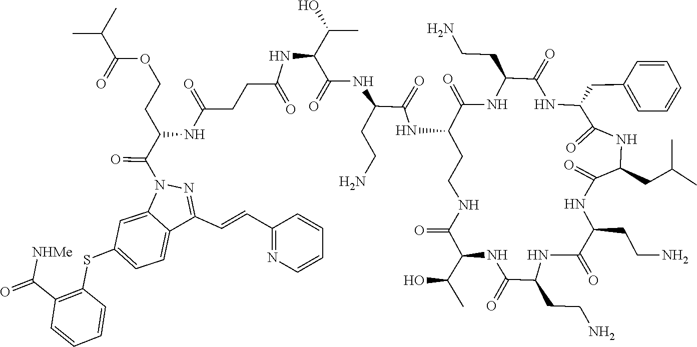 |
| 14 | 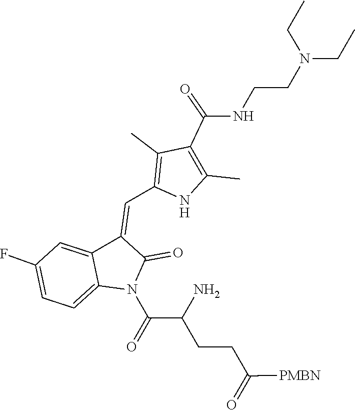 |
| 22 | 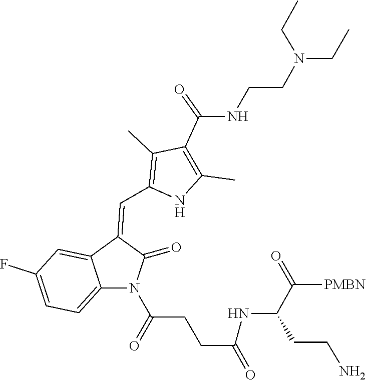 |
| 23 | 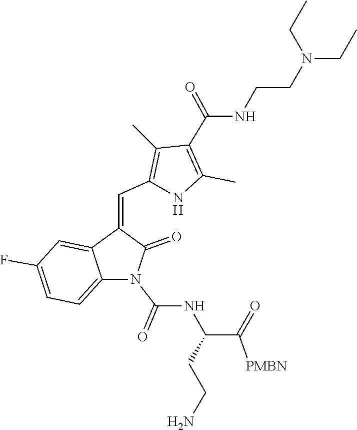 |
| 24 | 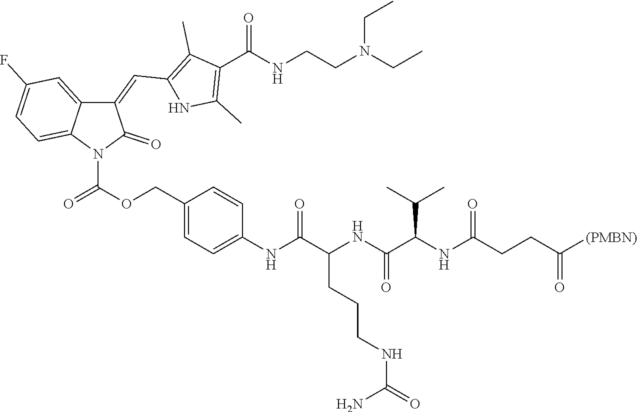 |
| 25 | 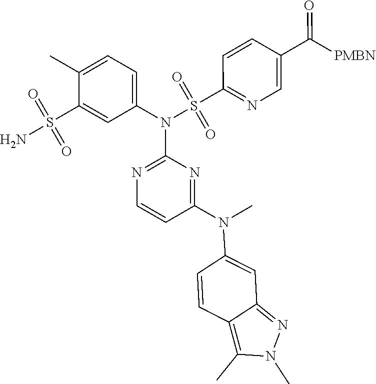 |
| 26 | 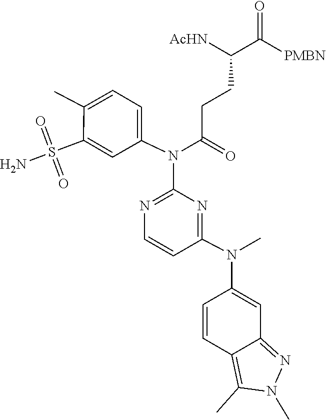 |
| 27 | 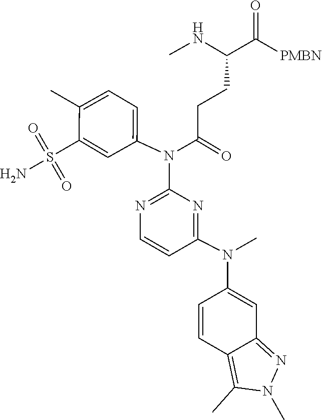 |
| 34 | 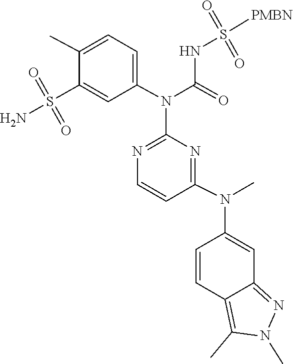 |
| 35 | 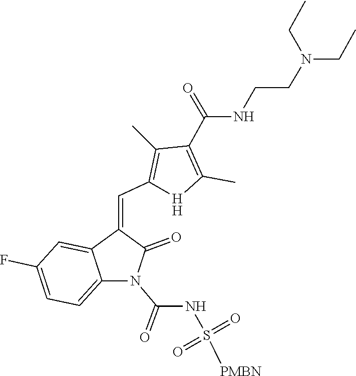 |
| 36 | 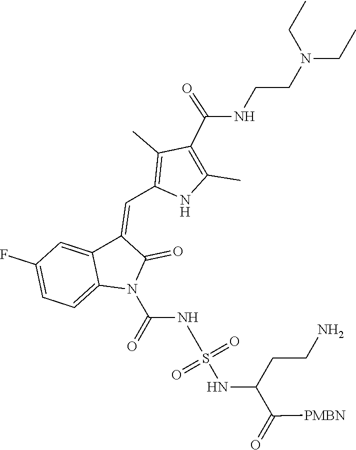 |
| 37 | 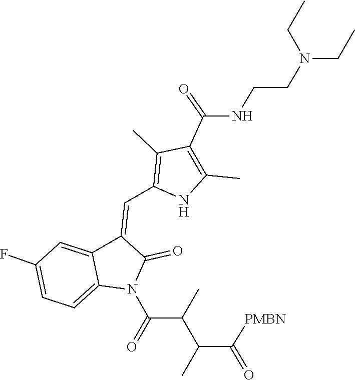 |
| 38 | 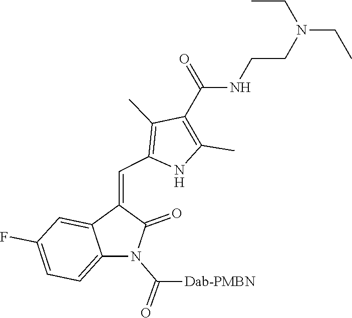 |
| 39 | 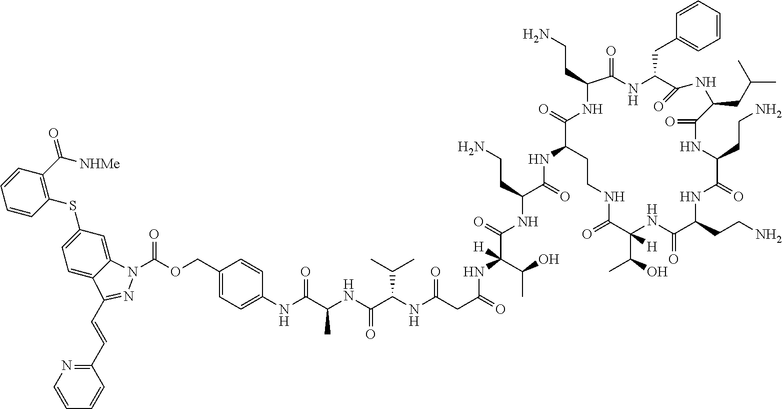 [SMILES: CNC(=O)c1ccccc1Sc1ccc2c(/C=C/c3ccccn3)nn(C(=O)OCc3ccc(NC(=O)[C@H](C)NC(=O)[C@@H](NC(=O)CC(=O)N[C@H](C(=O)N[C@@H](CCN)C(=O)N[C@@H]4CCNC(=O)[C@@H]([C@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@H](C)O)C(C)C)cc3)c2c1] |
| 46 | 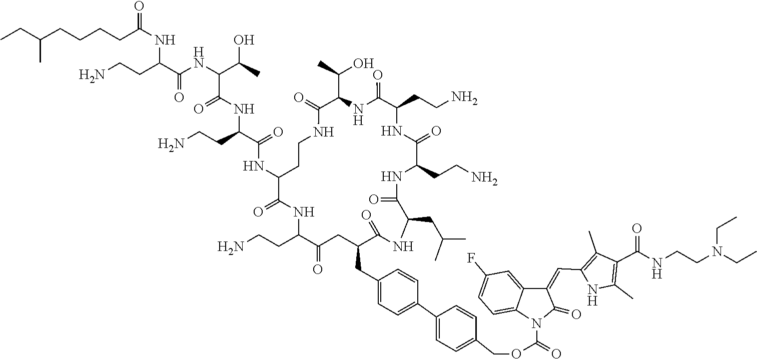 |
| 47 | 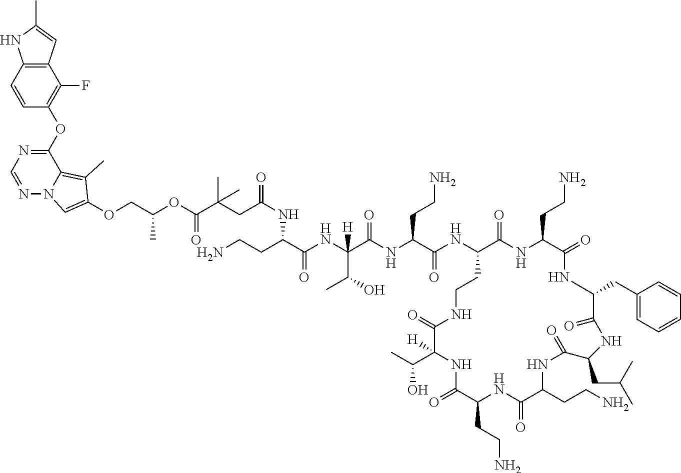 [SMILES: Cc1cc2c(F)c(Oc3ncnn4cc(OC[C@@H](C)OC(=O)C(C)(C)CC(=O)N[C@@H](CCN)C(=O)N[C@H](C(=O)N[C@@H](CCN)C(=O)N[C@H]5CCNC(=O)[C@H]([C@@H](C)O)NC(=O)[C@H](CCN)NC(=O)C(CCN)NC(=O)[C@H](CC(C)C)NC(=O)[C@@H](Cc6ccccc6)NC(=O)[C@H](CCN)NC5=O)[C@@H](C)O)c(C)c34)ccc2[nH]1] |
| 48 | 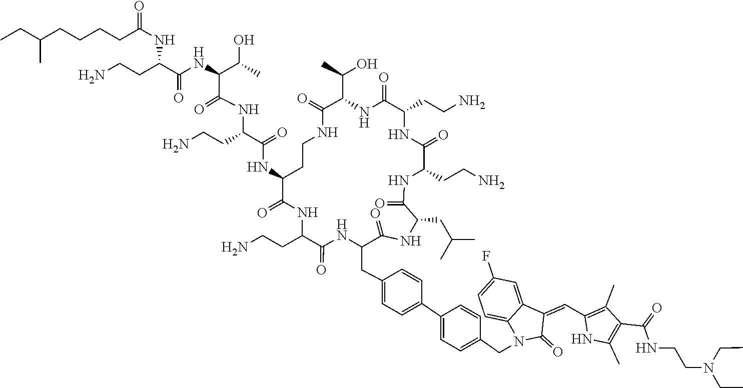 [SMILES: CCC(C)CCCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)C(Cc2ccc(-c3ccc(CN4C(=O)/C(=C\c5[nH]c(C)c(C(=O)NCCN(CC)CC)c5C)c5cc(F)ccc54)cc3)cc2)NC(=O)C(CCN)NC1=O)[C@@H](C)O] |
| 49 | 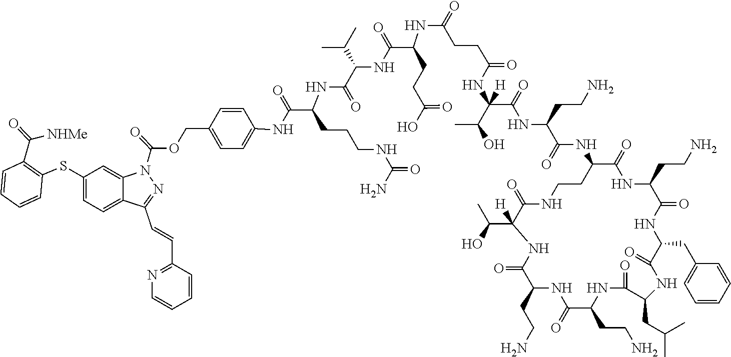 [SMILES: CNC(=O)c1ccccc1Sc1ccc2c(/C=C/c3ccccn3)nn(C(=O)OCc3ccc(NC(=O)[C@H](CCCNC(N)=O)NC(=O)[C@@H](NC(=O)[C@H](CCC(=O)O)NC(=O)CCC(=O)N[C@@H](C(=O)N[C@@H](CCN)C(=O)N[C@@H]4CCNC(=O)[C@@H]([C@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@H](C)O)C(C)C)cc3)c2c1] |
| 50 | 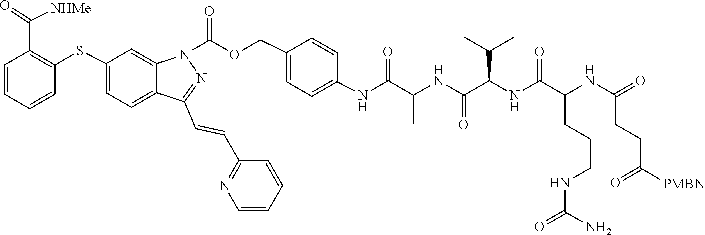 |
| 51 | 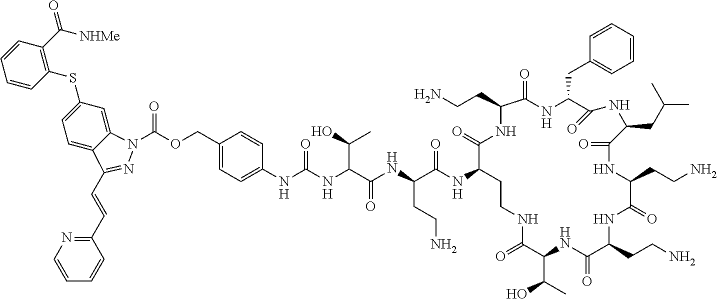 [SMILES: CNC(=O)c1ccccc1Sc1ccc2c(/C=C/c3ccccn3)nn(C(=O)OCc3ccc(NC(=O)NC(C(=O)N[C@H](CCN)C(=O)N[C@@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@H](C)O)cc3)c2c1] |
| 58 | 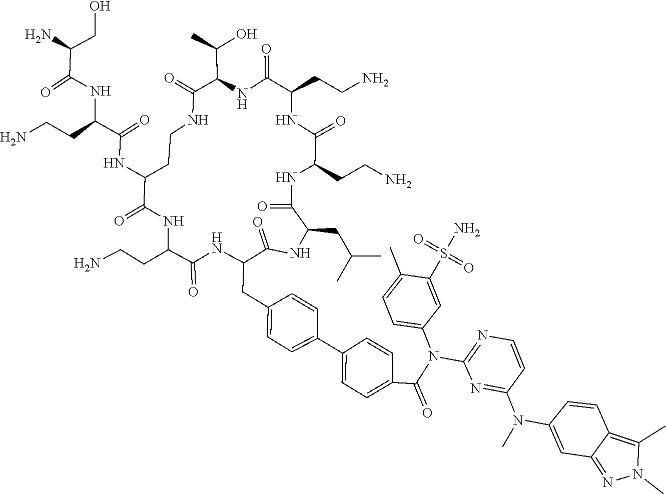 [SMILES: Cc1ccc(N(C(=O)c2ccc(-c3ccc(CC4NC(=O)C(CCN)NC(=O)C(NC(=O)[C@@H](CCN)NC(=O)[C@@H](N)CO)CCNC(=O)[C@@H]([C@@H](C)O)NC(=O)[C@@H](CCN)NC(=O)[C@@H](CCN)NC(=O)[C@@H](CC(C)C)NC4=O)cc3)cc2)c2nccc(N(C)c3ccc4c(C)n(C)nc4c3)n2)cc1S(N)(=O)=O] |
| 59 | 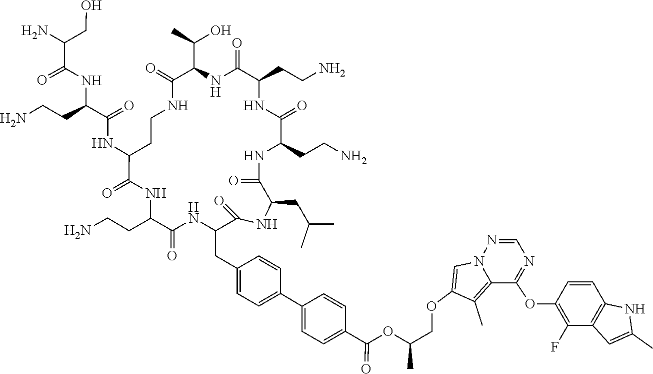 [SMILES: Cc1cc2c(F)c(Oc3ncnn4cc(OC[C@@H](C)OC(=O)c5ccc(-c6ccc(CC7NC(=O)C(CCN)NC(=O)C(NC(=O)[C@@H](CCN)NC(=O)C(N)CO)CCNC(=O)[C@@H]([C@@H](C)O)NC(=O)[C@@H](CCN)NC(=O)[C@@H](CCN)NC(=O)[C@@H](CC(C)C)NC7=O)cc6)cc5)c(C)c34)ccc2[nH]1] |
| 60 | 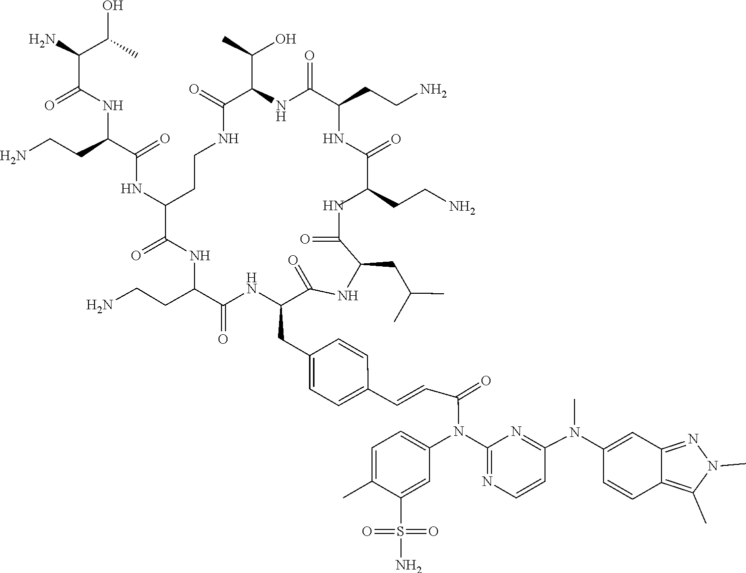 |
| 61 | 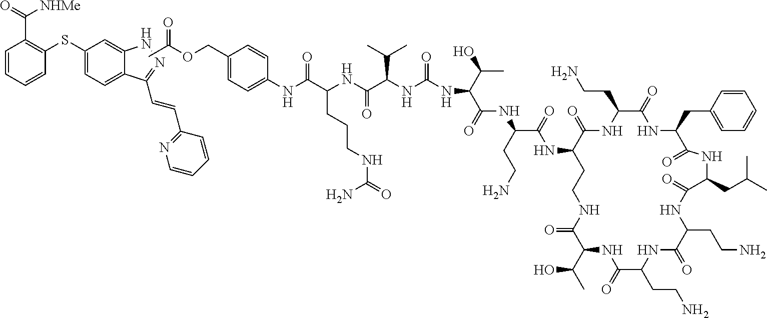 |
| 62 | 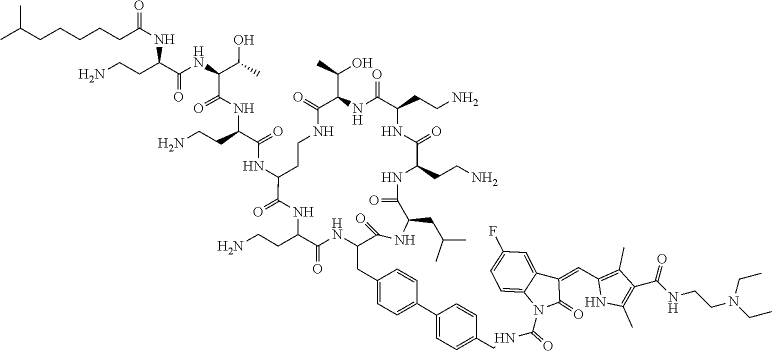 [SMILES: CCN(CC)CCNC(=O)c1c(C)[nH]c(/C=C2\C(=O)N(C(=O)NCc3ccc(-c4ccc(CC5NC(=O)C(CCN)NC(=O)C(NC(=O)[C@@H](CCN)NC(=O)[C@@H](NC(=O)[C@@H](CCN)NC(=O)CCCCCC(C)C)[C@@H](C)O)CCNC(=O)[C@@H]([C@@H](C)O)NC(=O)[C@@H](CCN)NC(=O)[C@@H](CCN)NC(=O)[C@@H](CC(C)C)NC5=O)cc4)cc3)c3ccc(F)cc32)c1C] |
| 63 | 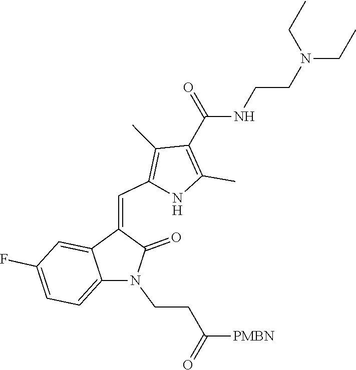 |
| 70 | 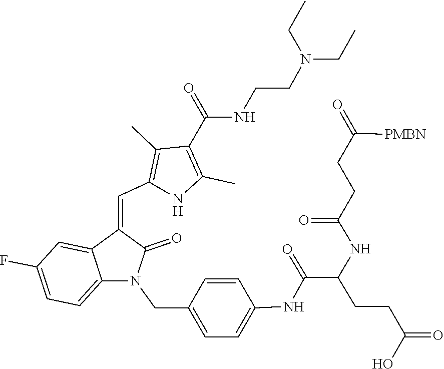 |
| 71 | 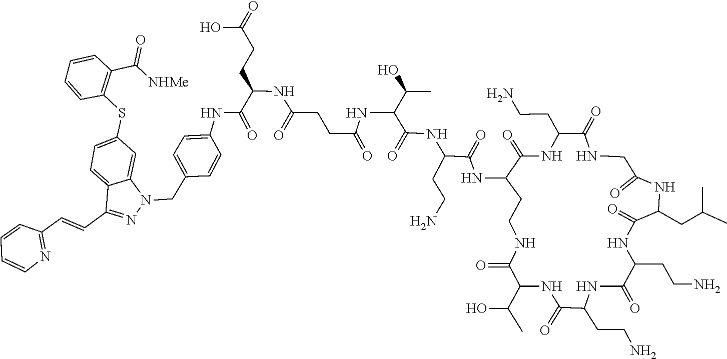 |
| 72 | 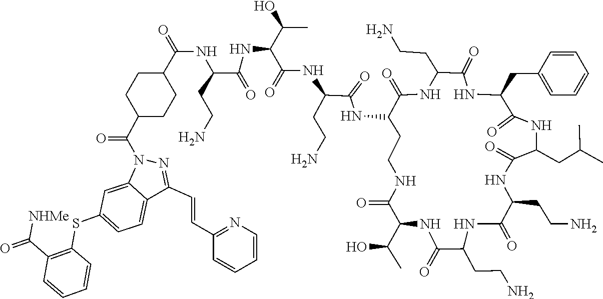 |
| 73 | 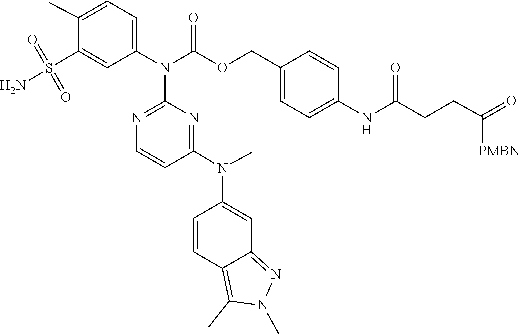 |
| 74 | 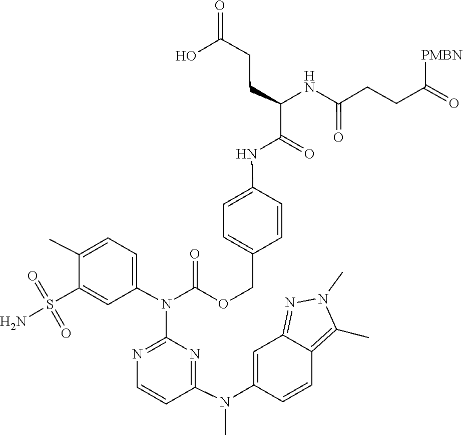 [SMILES: Cc1ccc(N(C(=O)OCc2ccc(NC(=O)[C@@H](CCC(=O)O)NC(=O)CCC=O)cc2)c2nccc(N(C)c3ccc4c(C)n(C)nc4c3)n2)cc1S(N)(=O)=O ; "PMBN"] |
| 75 | 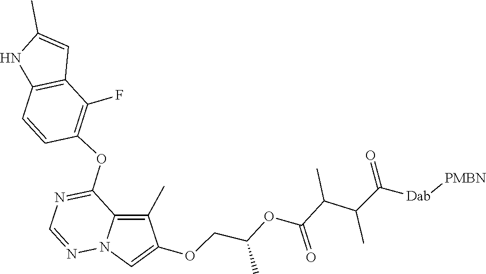 |
| 82 | 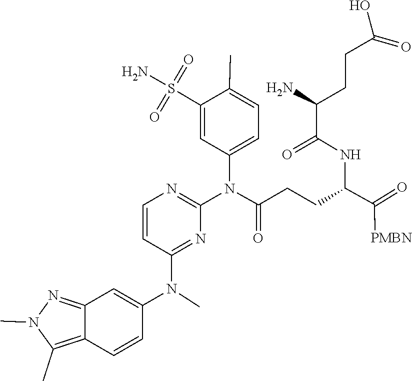 |
| 83 | 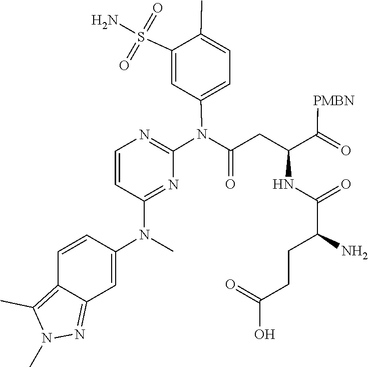 |
| 84 | 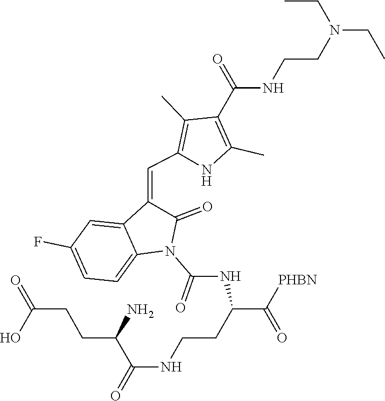 |
| 85 | 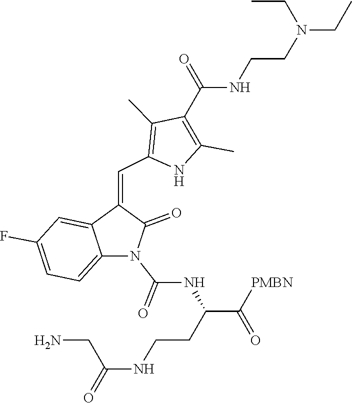 |
| 86 | 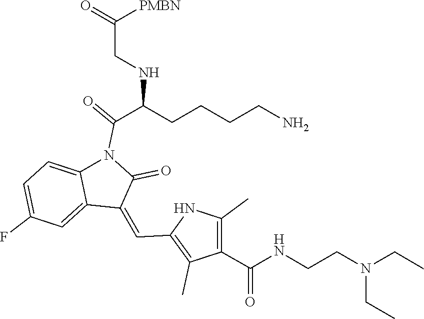 |
| 87 | 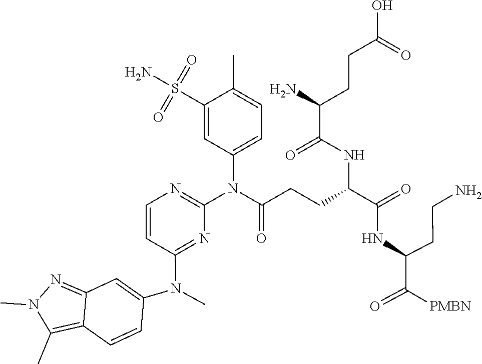 |
| 94 | 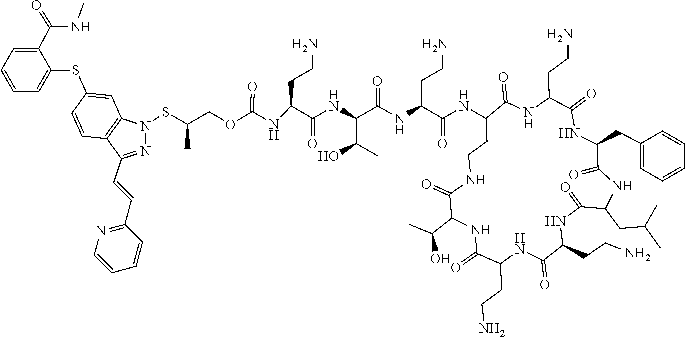 [SMILES: CNC(=O)c1ccccc1Sc1ccc2c(/C=C/c3ccccn3)nn(S[C@H](C)COC(=O)N[C@@H](CCN)C(=O)N[C@@H](C(=O)N[C@@H](CCN)C(=O)NC3CCNC(=O)C([C@H](C)O)NC(=O)C(CCN)NC(=O)[C@H](CCN)NC(=O)C(CC(C)C)NC(=O)[C@H](Cc4ccccc4)NC(=O)C(CCN)NC3=O)[C@@H](C)O)c2c1] |
| 95 | 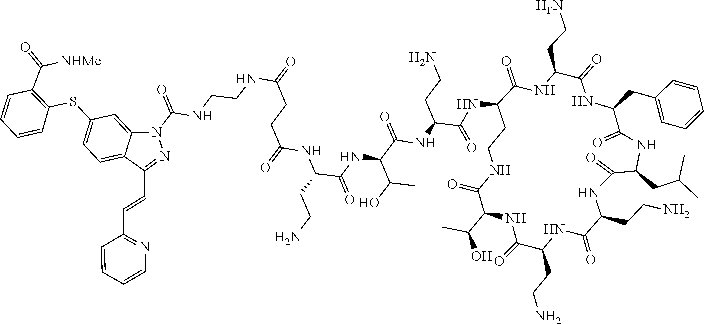 |
| 96 | 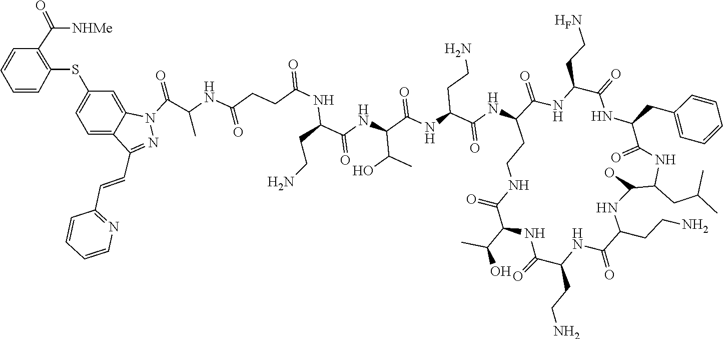 |
| 97 | 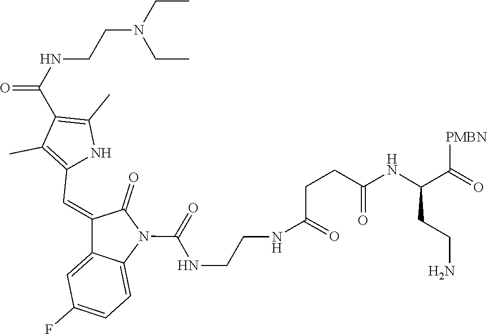 |
| 98 | 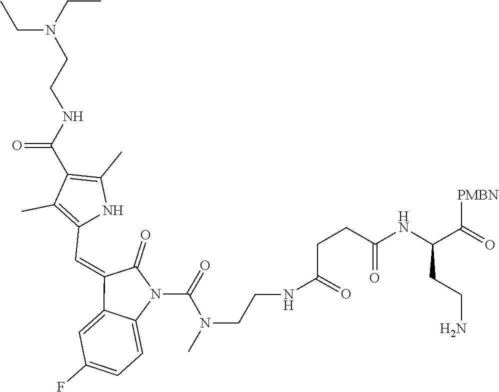 |
| 99 | 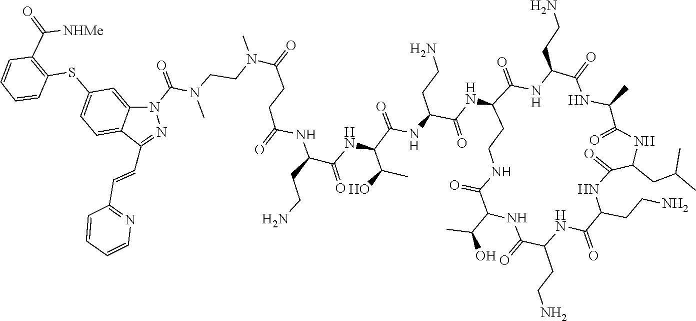 [SMILES: CNC(=O)c1ccccc1Sc1ccc2c(/C=C/c3ccccn3)nn(C(=O)N(C)CCN(C)C(=O)CCC(=O)N[C@H](CCN)C(=O)N[C@@H](C(=O)N[C@@H](CCN)C(=O)N[C@@H]3CCNC(=O)C([C@H](C)O)NC(=O)C(CCN)NC(=O)C(CCN)NC(=O)C(CC(C)C)NC(=O)[C@H](C)NC(=O)[C@H](CCN)NC3=O)[C@@H](C)O)c2c1] |
| 106 | 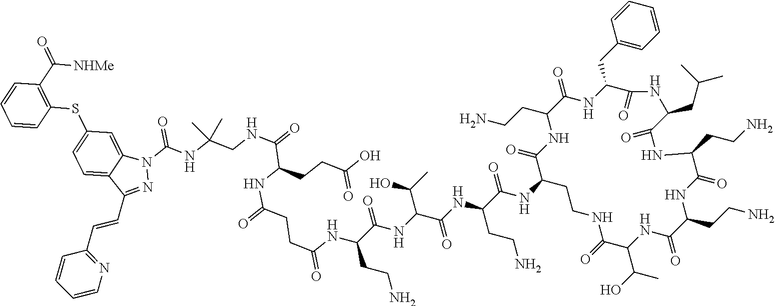 [SMILES: CNC(=O)c1ccccc1Sc1ccc2c(/C=C/c3ccccn3)nn(C(=O)NC(C)(C)CNC(=O)[C@@H](CCC(=O)O)NC(=O)CCC(=O)N[C@H](CCN)C(=O)NC(C(=O)N[C@H](CCN)C(=O)N[C@@H]3CCNC(=O)C(C(C)O)NC(=O)[C@H](CCN)NC(=O)[C@H](CCN)NC(=O)[C@H](CC(C)C)NC(=O)[C@@H](Cc4ccccc4)NC(=O)C(CCN)NC3=O)[C@H](C)O)c2c1] |
| 107 | 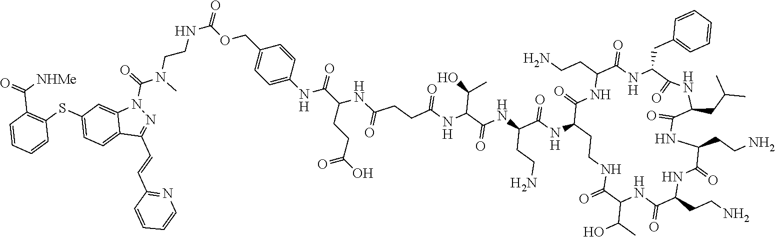 [SMILES: CNC(=O)c1ccccc1Sc1ccc2c(/C=C/c3ccccn3)nn(C(=O)N(C)CCNC(=O)OCc3ccc(NC(=O)C(CCC(=O)O)NC(=O)CCC(=O)NC(C(=O)N[C@H](CCN)C(=O)N[C@@H]4CCNC(=O)C(C(C)O)NC(=O)[C@H](CCN)NC(=O)[C@H](CCN)NC(=O)[C@H](CC(C)C)NC(=O)[C@@H](Cc5ccccc5)NC(=O)C(CCN)NC4=O)[C@H](C)O)cc3)c2c1] |
| 108 | 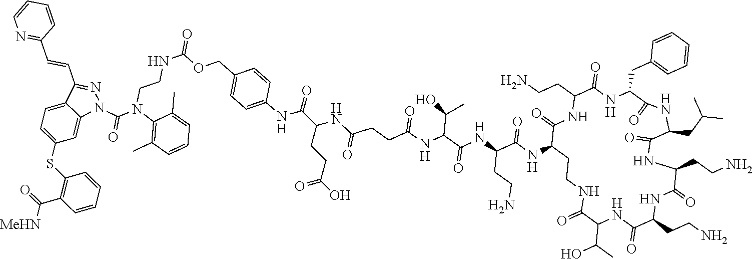 [SMILES: CNC(=O)c1ccccc1Sc1ccc2c(/C=C/c3ccccn3)nn(C(=O)N(CCNC(=O)OCc3ccc(NC(=O)C(CCC(=O)O)NC(=O)CCC(=O)NC(C(=O)N[C@H](CCN)C(=O)N[C@@H]4CCNC(=O)C(C(C)O)NC(=O)[C@H](CCN)NC(=O)[C@H](CCN)NC(=O)[C@H](CC(C)C)NC(=O)[C@@H](Cc5ccccc5)NC(=O)C(CCN)NC4=O)[C@H](C)O)cc3)c3c(C)cccc3C)c2c1] |
| 109 | 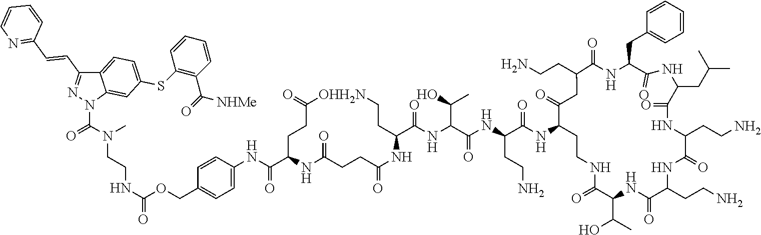 [SMILES: CNC(=O)c1ccccc1Sc1ccc2c(/C=C/c3ccccn3)nn(C(=O)N(C)CCNC(=O)OCc3ccc(NC(=O)[C@@H](CCC(=O)O)NC(=O)CCC(=O)N[C@@H](CCN)C(=O)NC(C(=O)N[C@H](CCN)C(=O)N[C@@H]4CCNC(=O)[C@H](C(C)O)NC(=O)C(CCN)NC(=O)C(CCN)NC(=O)C(CC(C)C)NC(=O)[C@H](Cc5ccccc5)NC(=O)C(CCN)CC4=O)[C@H](C)O)cc3)c2c1] |
| 110 | 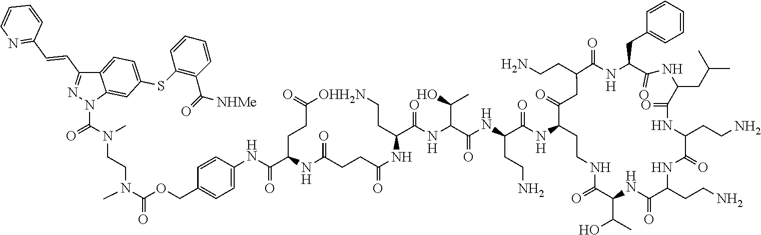 [SMILES: CNC(=O)c1ccccc1Sc1ccc2c(/C=C/c3ccccn3)nn(C(=O)N(C)CCN(C)C(=O)OCc3ccc(NC(=O)[C@@H](CCC(=O)O)NC(=O)CCC(=O)N[C@@H](CCN)C(=O)NC(C(=O)N[C@H](CCN)C(=O)N[C@@H]4CCNC(=O)[C@H](C(C)O)NC(=O)C(CCN)NC(=O)C(CCN)NC(=O)C(CC(C)C)NC(=O)[C@H](Cc5ccccc5)NC(=O)C(CCN)CC4=O)[C@H](C)O)cc3)c2c1] |
| 111 | 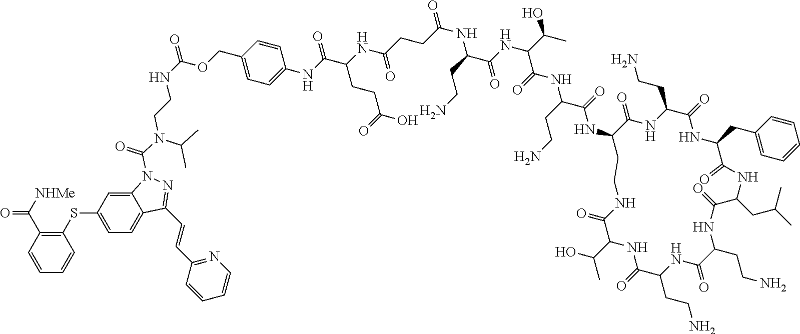 [SMILES: CNC(=O)c1ccccc1Sc1ccc2c(/C=C/c3ccccn3)nn(C(=O)N(CCNC(=O)OCc3ccc(NC(=O)C(CCC(=O)O)NC(=O)CCC(=O)N[C@H](CCN)C(=O)NC(C(=O)NC(CCN)C(=O)N[C@@H]4CCNC(=O)C(C(C)O)NC(=O)C(CCN)NC(=O)C(CCN)NC(=O)C(CC(C)C)NC(=O)[C@H](Cc5ccccc5)NC(=O)[C@H](CCN)NC4=O)[C@H](C)O)cc3)C(C)C)c2c1] |
| 118 | 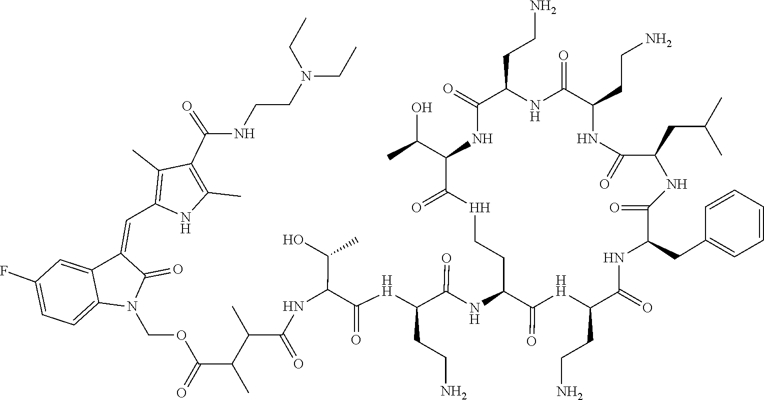 |
| 119 | 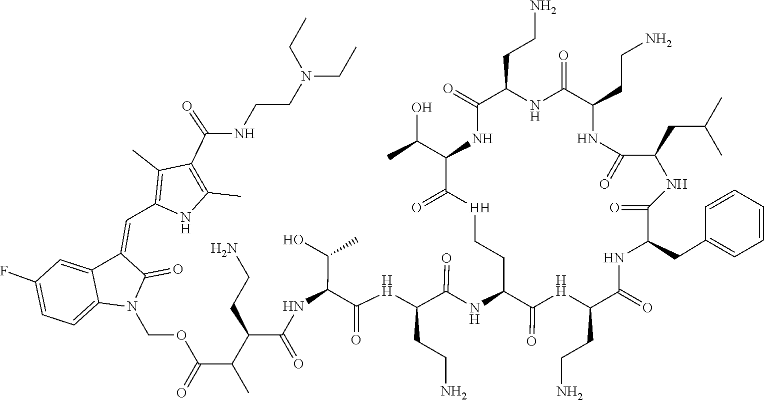 |
| 120 | 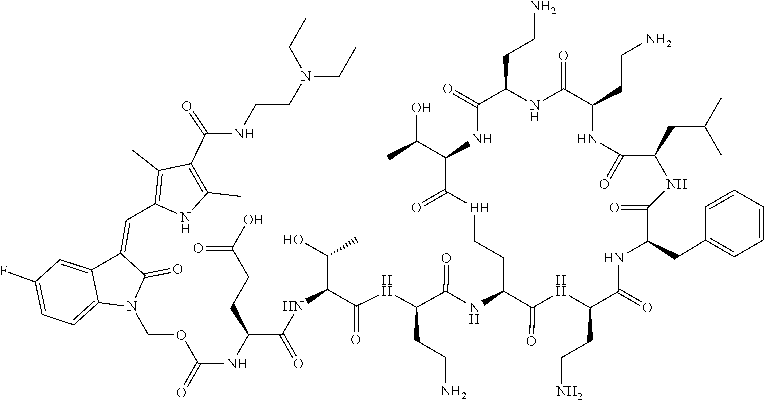 |
| 121 | 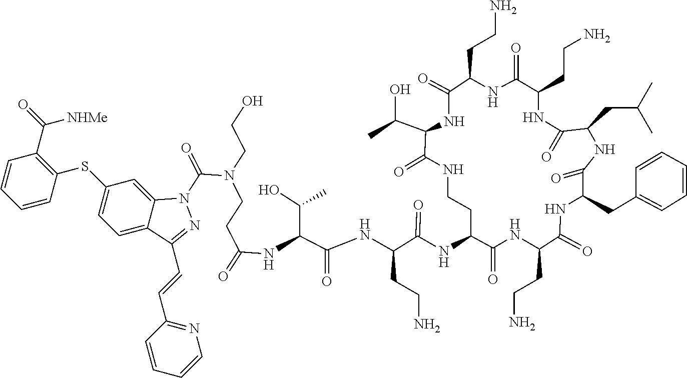 [SMILES: CNC(=O)c1ccccc1Sc1ccc2c(/C=C/c3ccccn3)nn(C(=O)N(CCO)CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 122 | 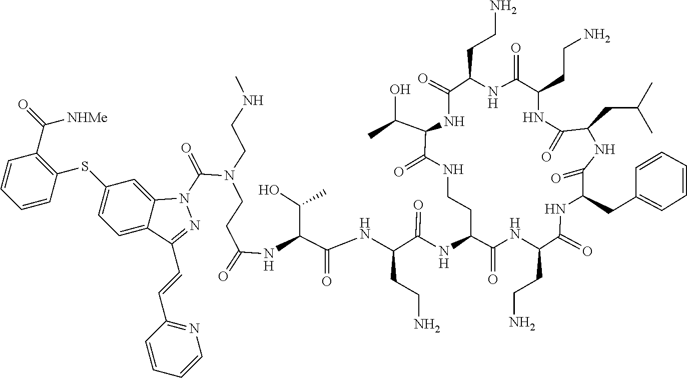 [SMILES: CNCCN(CCC(=O)N[C@H](C(=O)N[C@H](CCN)C(=O)N[C@H]1CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc2ccccc2)NC(=O)[C@@H](CCN)NC1=O)[C@@H](C)O)C(=O)n1nc(/C=C/c2ccccn2)c2ccc(Sc3ccccc3C(=O)NC)cc21] |
| 123 | 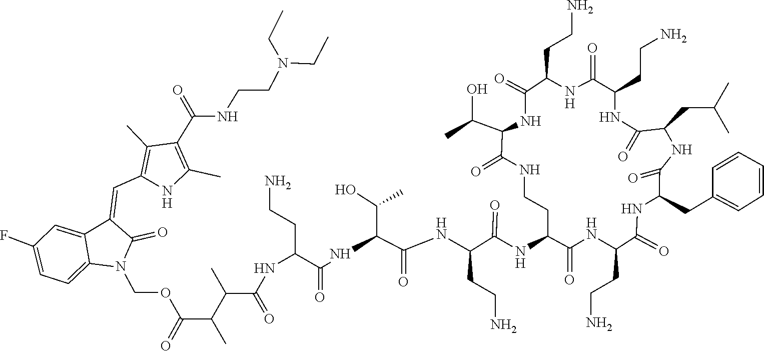 [SMILES: CCN(CC)CCNC(=O)c1c(C)[nH]c(/C=C2\C(=O)N(COC(=O)C(C)C(C)C(=O)NC(CCN)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 130 | 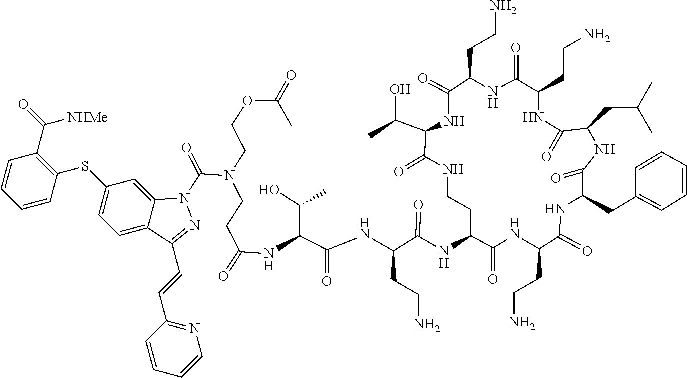 [SMILES: CNC(=O)c1ccccc1Sc1ccc2c(/C=C/c3ccccn3)nn(C(=O)N(CCOC(C)=O)CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 131 | 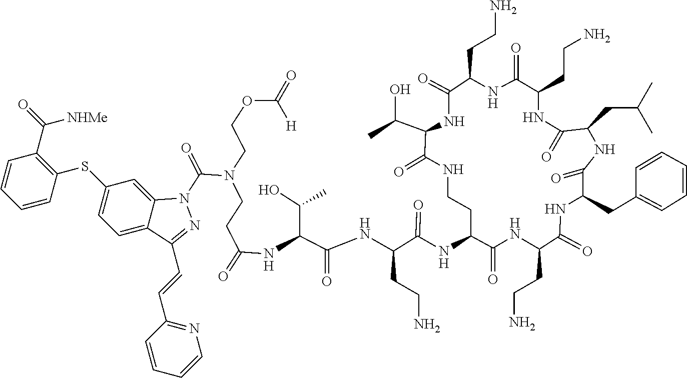 [SMILES: CNC(=O)c1ccccc1Sc1ccc2c(/C=C/c3ccccn3)nn(C(=O)N(CCOC=O)CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 132 | 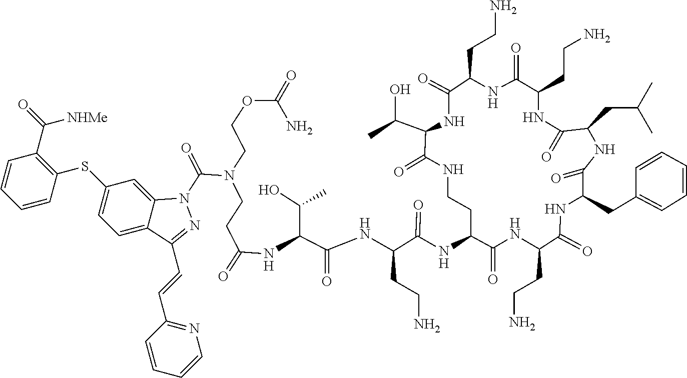 [SMILES: CNC(=O)c1ccccc1Sc1ccc2c(/C=C/c3ccccn3)nn(C(=O)N(CCOC(N)=O)CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 133 | 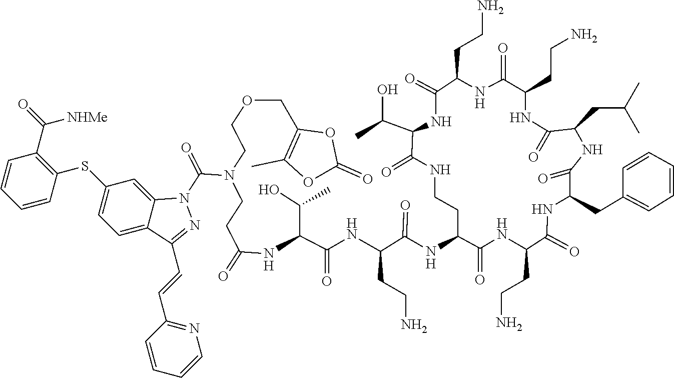 [SMILES: CNC(=O)c1ccccc1Sc1ccc2c(/C=C/c3ccccn3)nn(C(=O)N(CCOCc3oc(=O)oc3C)CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 132 | 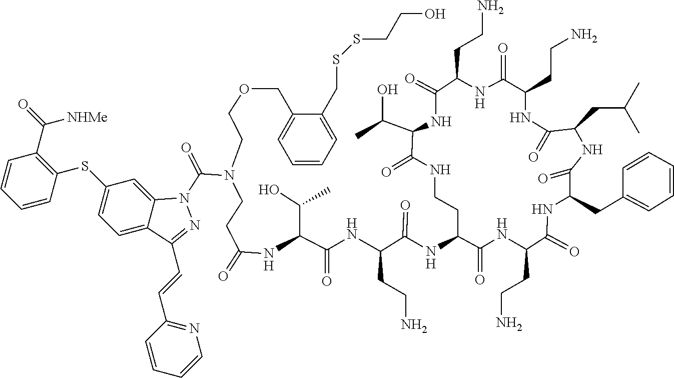 [SMILES: CNC(=O)c1ccccc1Sc1ccc2c(/C=C/c3ccccn3)nn(C(=O)N(CCOCc3ccccc3CSSCCO)CCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c2c1] |
| 133 | 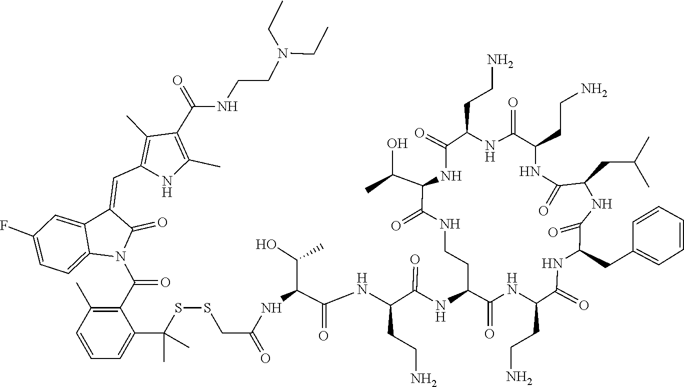 [SMILES: CCN(CC)CCNC(=O)c1c(C)[nH]c(/C=C2\C(=O)N(C(=O)c3c(C)cccc3C(C)(C)SSCC(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 140 | 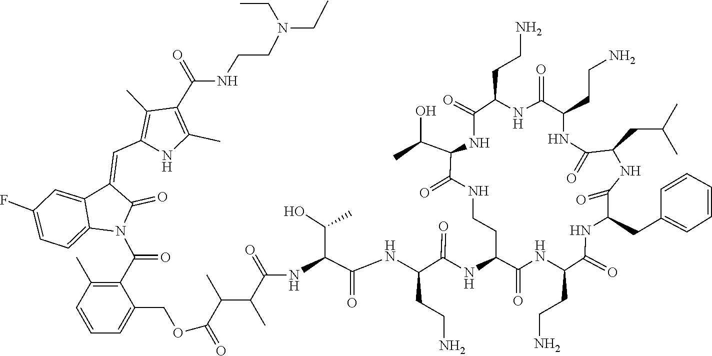 [SMILES: CCN(CC)CCNC(=O)c1c(C)[nH]c(/C=C2\C(=O)N(C(=O)c3c(C)cccc3COC(=O)C(C)C(C)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 141 | 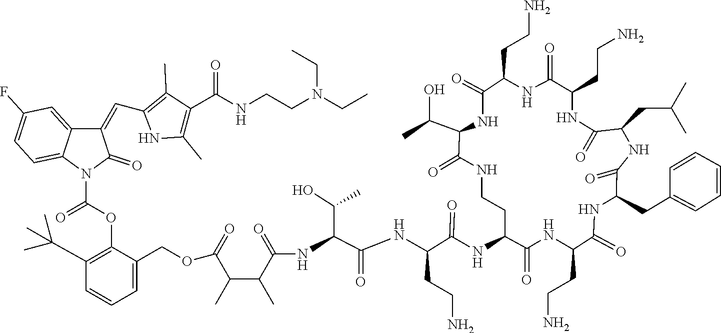 [SMILES: CCN(CC)CCNC(=O)c1c(C)[nH]c(/C=C2\C(=O)N(C(=O)Oc3c(COC(=O)C(C)C(C)C(=O)N[C@H](C(=O)N[C@H](CCN)C(=O)N[C@H]4CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc5ccccc5)NC(=O)[C@@H](CCN)NC4=O)[C@@H](C)O)cccc3C(C)(C)C)c3ccc(F)cc32)c1C] |
| 142 | 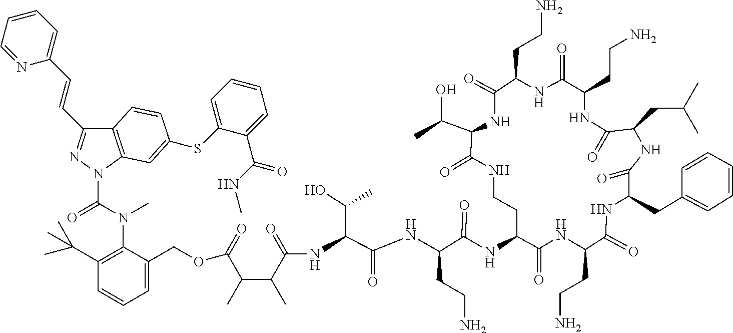 [SMILES: CNC(=O)c1ccccc1Sc1ccc2c(/C=C/c3ccccn3)nn(C(=O)N(C)c3c(COC(=O)C(C)C(C)C(=O)N[C@H](C(=O)N[C@H](CCN)C(=O)N[C@H]4CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc5ccccc5)NC(=O)[C@@H](CCN)NC4=O)[C@@H](C)O)cccc3C(C)(C)C)c2c1] |
| 143 | 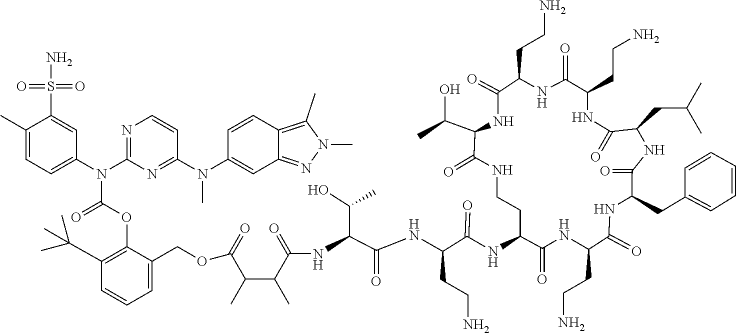 [SMILES: Cc1ccc(N(C(=O)Oc2c(COC(=O)C(C)C(C)C(=O)N[C@H](C(=O)N[C@H](CCN)C(=O)N[C@H]3CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)cccc2C(C)(C)C)c2nccc(N(C)c3ccc4c(C)n(C)nc4c3)n2)cc1S(N)(=O)=O] |
| 144 | 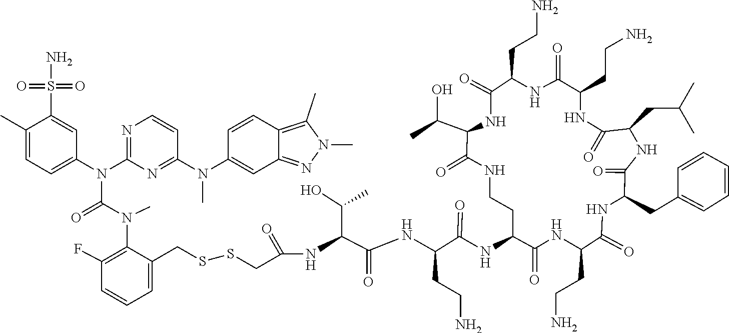 [SMILES: Cc1ccc(N(C(=O)N(C)c2c(F)cccc2CSSCC(=O)N[C@H](C(=O)N[C@H](CCN)C(=O)N[C@H]2CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc3ccccc3)NC(=O)[C@@H](CCN)NC2=O)[C@@H](C)O)c2nccc(N(C)c3ccc4c(C)n(C)nc4c3)n2)cc1S(N)(=O)=O] |
| 145 | 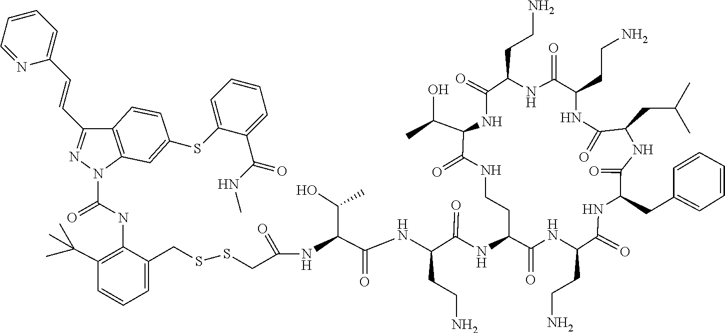 [SMILES: CNC(=O)c1ccccc1Sc1ccc2c(/C=C/c3ccccn3)nn(C(=O)Nc3c(CSSCC(=O)N[C@H](C(=O)N[C@H](CCN)C(=O)N[C@H]4CCNC(=O)[C@@H]([C@@H](C)O)NC(=O)[C@@H](CCN)NC(=O)[C@@H](CCN)NC(=O)[C@@H](CC(C)C)NC(=O)[C@@H](Cc5ccccc5)NC(=O)[C@@H](CCN)NC4=O)[C@@H](C)O)cccc3C(C)(C)C)c2c1] |
| 148 | 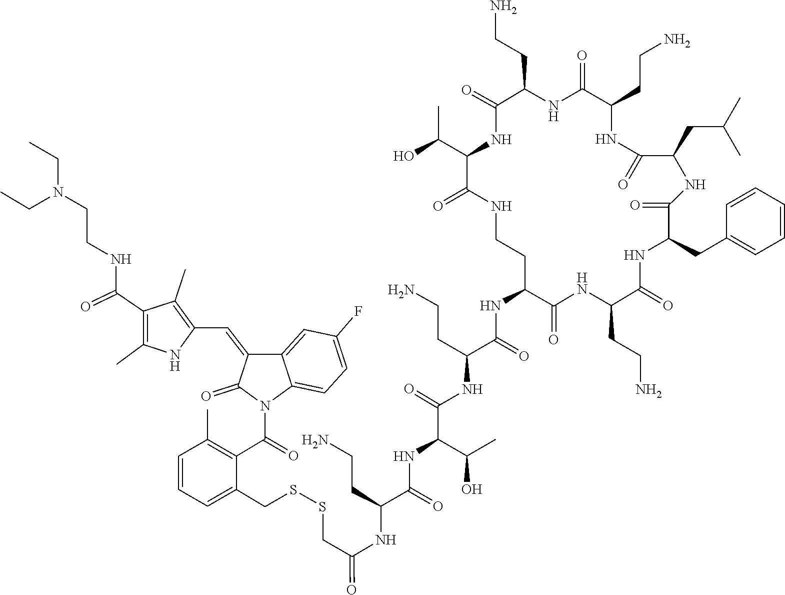 [SMILES: CCN(CC)CCNC(=O)c1c(C)[nH]c(/C=C2\C(=O)N(C(=O)c3c(C)cccc3CSSCC(=O)N[C@@H](CCN)C(=O)N[C@@H](C(=O)N[C@@H](CCN)C(=O)N[C@H]3CCNC(=O)[C@@H]([C@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)[C@@H](C)O)c3ccc(F)cc32)c1C] |
| 149 | 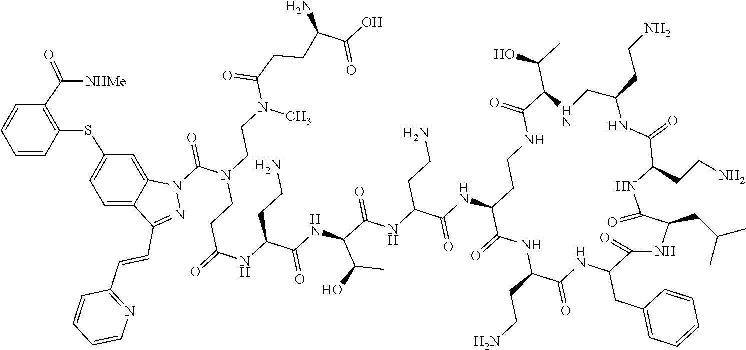 [SMILES: CNC(=O)c1ccccc1Sc1ccc2c(/C=C/c3ccccn3)nn(C(=O)N(CCC(=O)N[C@@H](CCN)C(=O)N[C@@H](C(=O)NC(CCN)C(=O)N[C@H]3CCNC(=O)[C@@H]([C@H](C)O)NC[C@@H](CCN)NC(=O)[C@@H](CCN)NC(=O)[C@@H](CC(C)C)NC(=O)C(Cc4ccccc4)NC(=O)[C@@H](CCN)NC3=O)[C@@H](C)O)CCN(C)C(=O)CC[C@@H](N)C(=O)O)c2c1] |
| 154 | 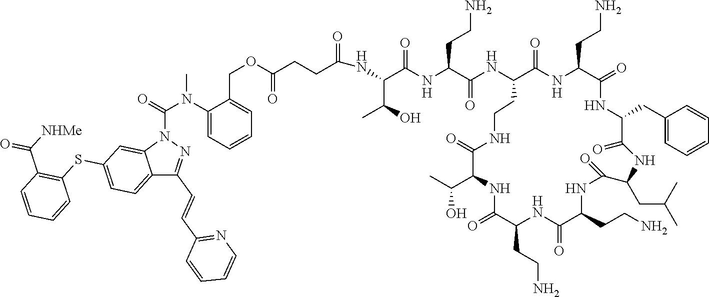 [SMILES: CNC(=O)c1ccccc1Sc1ccc2c(/C=C/c3ccccn3)nn(C(=O)N(C)c3ccccc3COC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 155 | 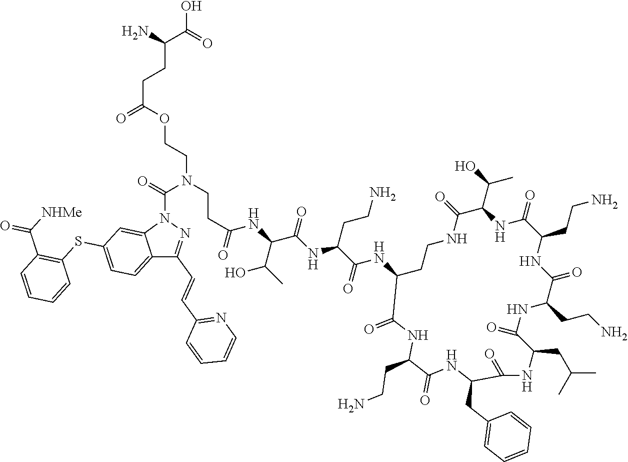 [SMILES: CNC(=O)c1ccccc1Sc1ccc2c(/C=C/c3ccccn3)nn(C(=O)N(CCOC(=O)CC[C@@H](N)C(=O)O)CCC(=O)N[C@@H](C(=O)N[C@@H](CCN)C(=O)N[C@H]3CCNC(=O)[C@@H]([C@H](C)O)NC(=O)[C@@H](CCN)NC(=O)[C@@H](CCN)NC(=O)[C@@H](CC(C)C)NC(=O)[C@@H](Cc4ccccc4)NC(=O)[C@@H](CCN)NC3=O)C(C)O)c2c1] |
| 156 | 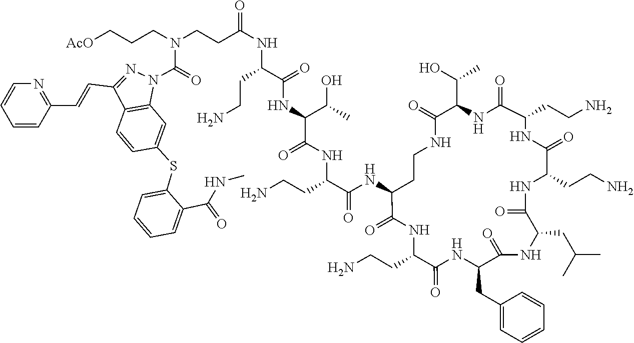 [SMILES: CNC(=O)c1ccccc1Sc1ccc2c(/C=C/c3ccccn3)nn(C(=O)N(CCCOC(C)=O)CCC(=O)N[C@@H](CCN)C(=O)N[C@H](C(=O)N[C@@H](CCN)C(=O)N[C@H]3CCNC(=O)[C@@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 157 | 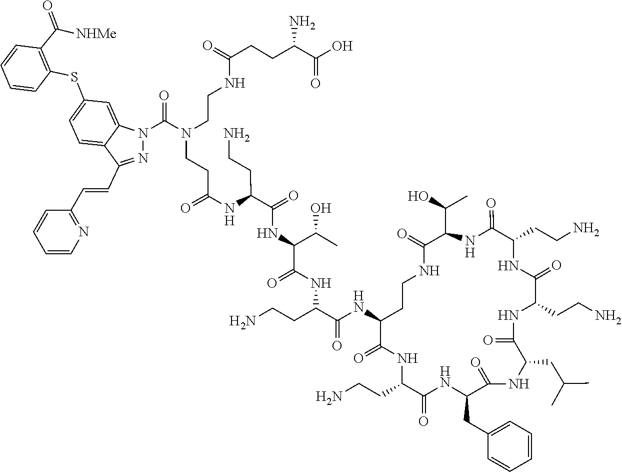 |
| 164 | 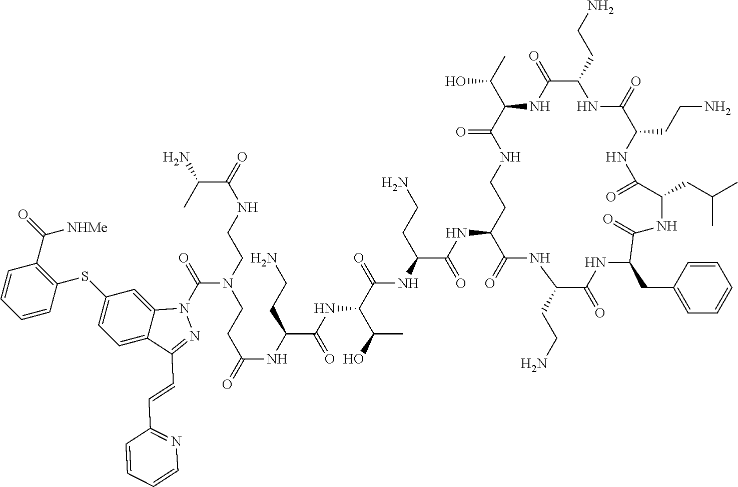 [SMILES: CNC(=O)c1ccccc1Sc1ccc2c(/C=C/c3ccccn3)nn(C(=O)N(CCNC(=O)[C@H](C)N)CCC(=O)N[C@@H](CCN)C(=O)N[C@H](C(=O)N[C@@H](CCN)C(=O)N[C@H]3CCNC(=O)[C@@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 165 | 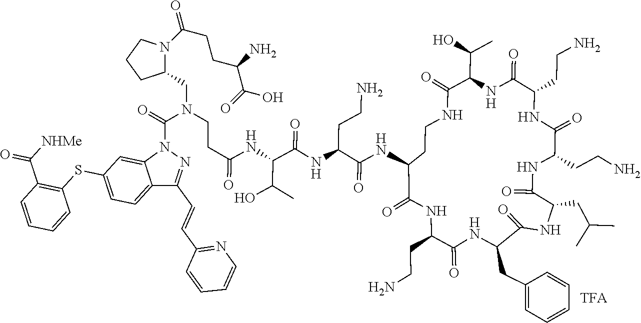 [SMILES: CNC(=O)c1ccccc1Sc1ccc2c(/C=C/c3ccccn3)nn(C(=O)N(CCC(=O)N[C@H](C(=O)N[C@@H](CCN)C(=O)N[C@H]3CCNC(=O)[C@@H]([C@H](C)O)NC(=O)[C@H](CCN)NC(=O)[C@H](CCN)NC(=O)[C@H](CC(C)C)NC(=O)[C@@H](Cc4ccccc4)NC(=O)[C@@H](CCN)NC3=O)C(C)O)C[C@@H]3CCCN3C(=O)CC[C@@H](N)C(=O)O)c2c1] |
| 166 | 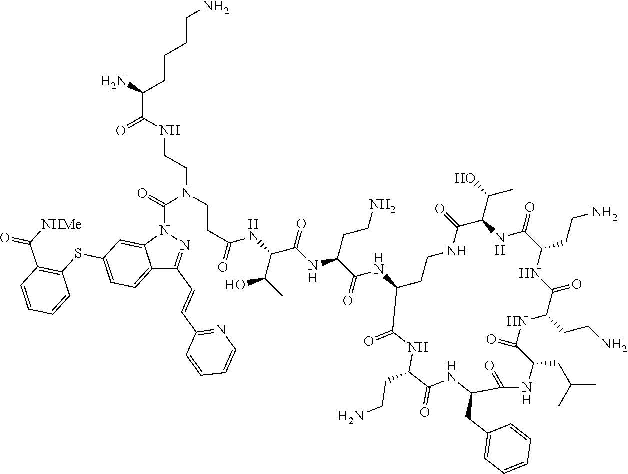 [SMILES: CNC(=O)c1ccccc1Sc1ccc2c(/C=C/c3ccccn3)nn(C(=O)N(CCNC(=O)[C@@H](N)CCCCN)CCC(=O)N[C@H](C(=O)N[C@@H](CCN)C(=O)N[C@H]3CCNC(=O)[C@@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 167 | 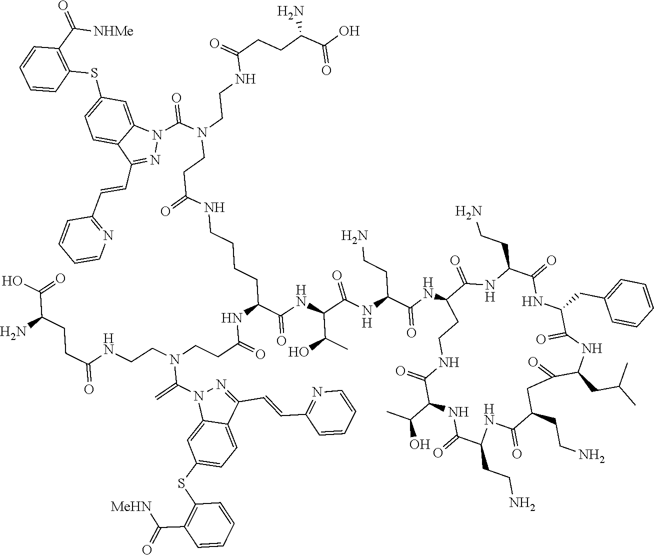 [SMILES: C=C(N(CCNC(=O)CC[C@@H](N)C(=O)O)CCC(=O)N[C@@H](CCCCNC(=O)CCN(CCNC(=O)CC[C@H](N)C(=O)O)C(=O)n1nc(/C=C/c2ccccn2)c2ccc(Sc3ccccc3C(=O)NC)cc21)C(=O)N[C@@H](C(=O)N[C@@H](CCN)C(=O)N[C@@H]1CCNC(=O)[C@H]([C@H](C)O)NC(=O)[C@H](CCN)NC(=O)[C@H](CCN)CC(=O)[C@H](CC(C)C)NC(=O)[C@@H](Cc2ccccc2)NC(=O)[C@H](CCN)NC1=O)[C@@H](C)O)n1nc(/C=C/c2ccccn2)c2ccc(Sc3ccccc3C(=O)NC)cc21] |
| 168 | 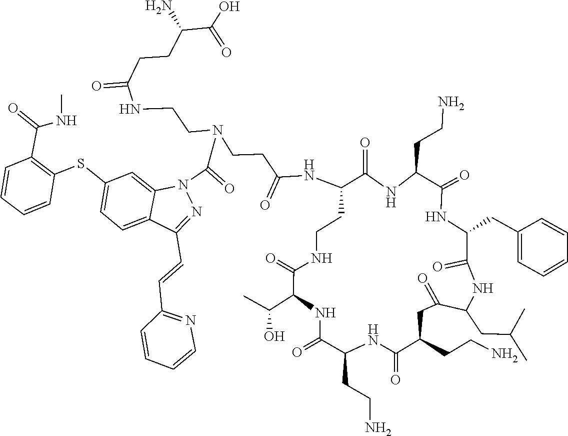 [SMILES: CNC(=O)c1ccccc1Sc1ccc2c(/C=C/c3ccccn3)nn(C(=O)N(CCNC(=O)CC[C@H](N)C(=O)O)CCC(=O)N[C@H]3CCNC(=O)[C@H]([C@@H](C)O)NC(=O)[C@H](CCN)NC(=O)[C@H](CCN)CC(=O)C(CC(C)C)NC(=O)[C@@H](Cc4ccccc4)NC(=O)[C@H](CCN)NC3=O)c2c1] |
| 169 | 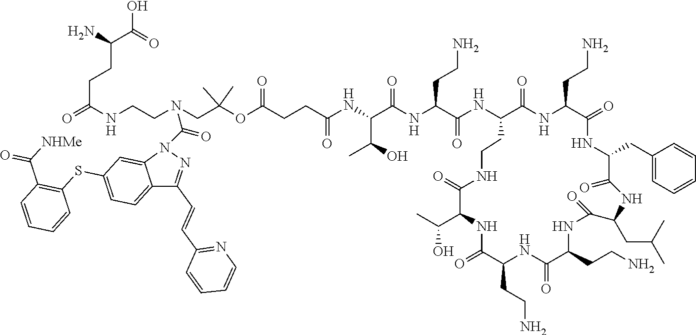 [SMILES: CNC(=O)c1ccccc1Sc1ccc2c(/C=C/c3ccccn3)nn(C(=O)N(CCNC(=O)CC[C@@H](N)C(=O)O)CC(C)(C)OC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 171 | 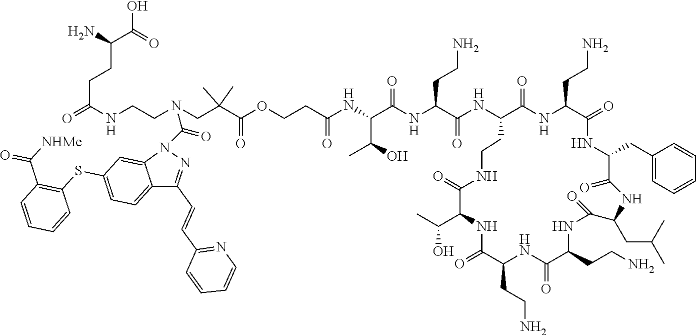 [SMILES: CNC(=O)c1ccccc1Sc1ccc2c(/C=C/c3ccccn3)nn(C(=O)N(CCNC(=O)CC[C@@H](N)C(=O)O)CC(C)(C)C(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 174 | 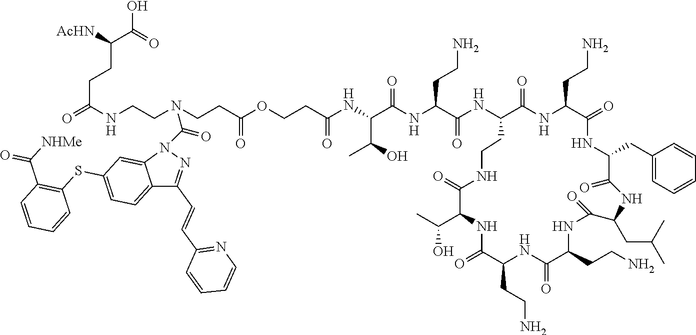 [SMILES: CNC(=O)c1ccccc1Sc1ccc2c(/C=C/c3ccccn3)nn(C(=O)N(CCNC(=O)CC[C@@H](NC(C)=O)C(=O)O)CCC(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)c2c1] |
| 175 | 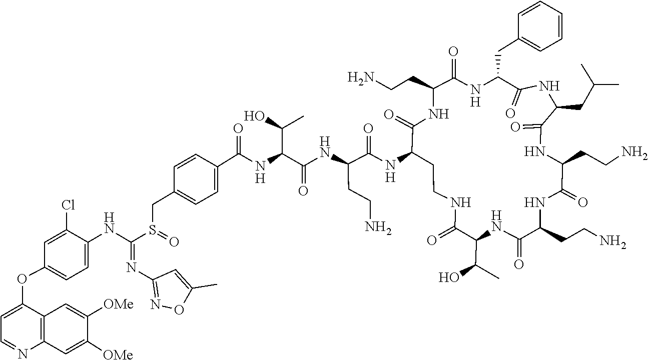 [SMILES: COc1cc2nccc(Oc3ccc(N/C(=N/c4cc(C)on4)S(=O)Cc4ccc(C(=O)N[C@H](C(=O)N[C@H](CCN)C(=O)N[C@@H]5CCNC(=O)[C@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@H](C)O)cc4)c(Cl)c3)c2cc1OC] |

In one preferred embodiment, the compound is selected from the structures below, or a pharmaceutically acceptable salt, solvate, or hydrate thereof:

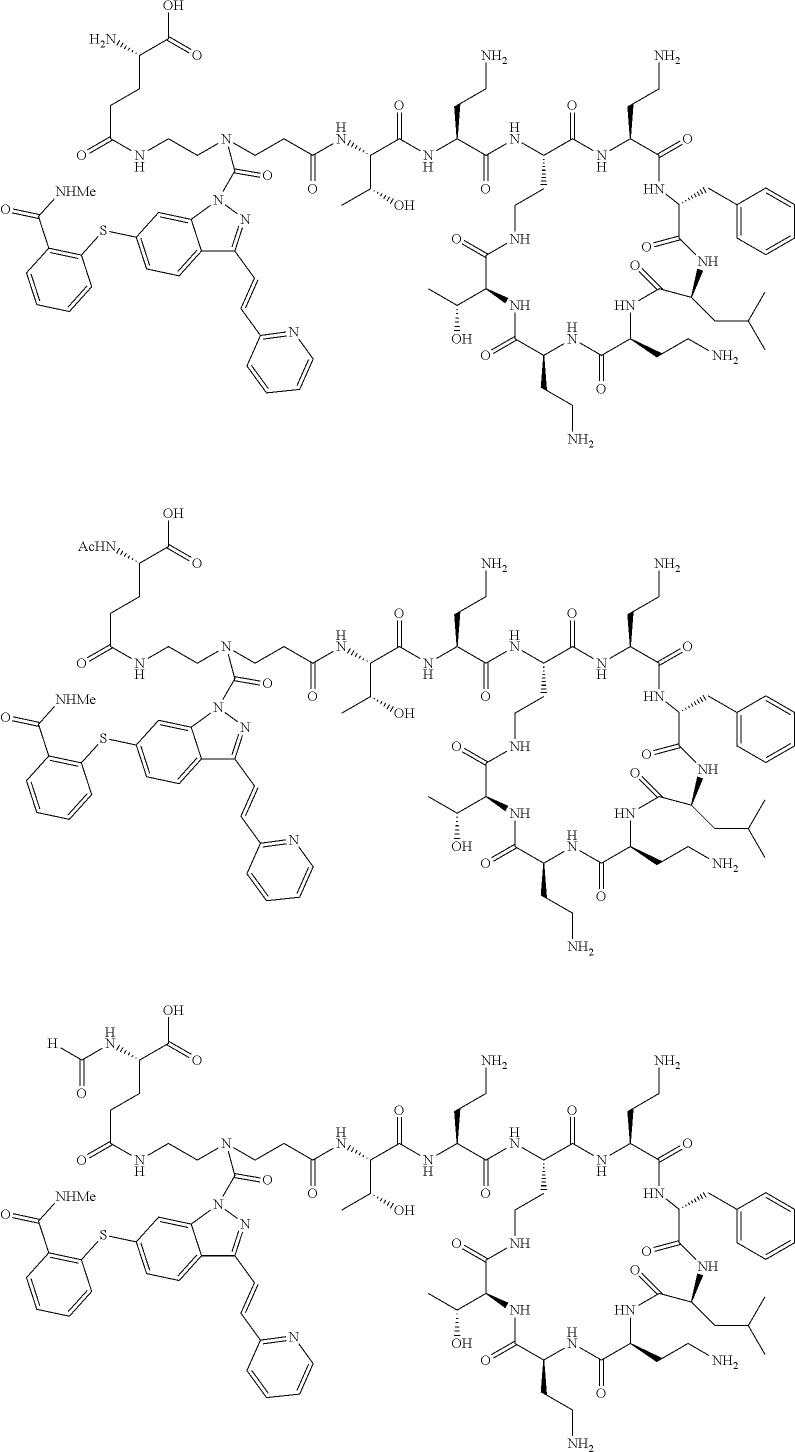

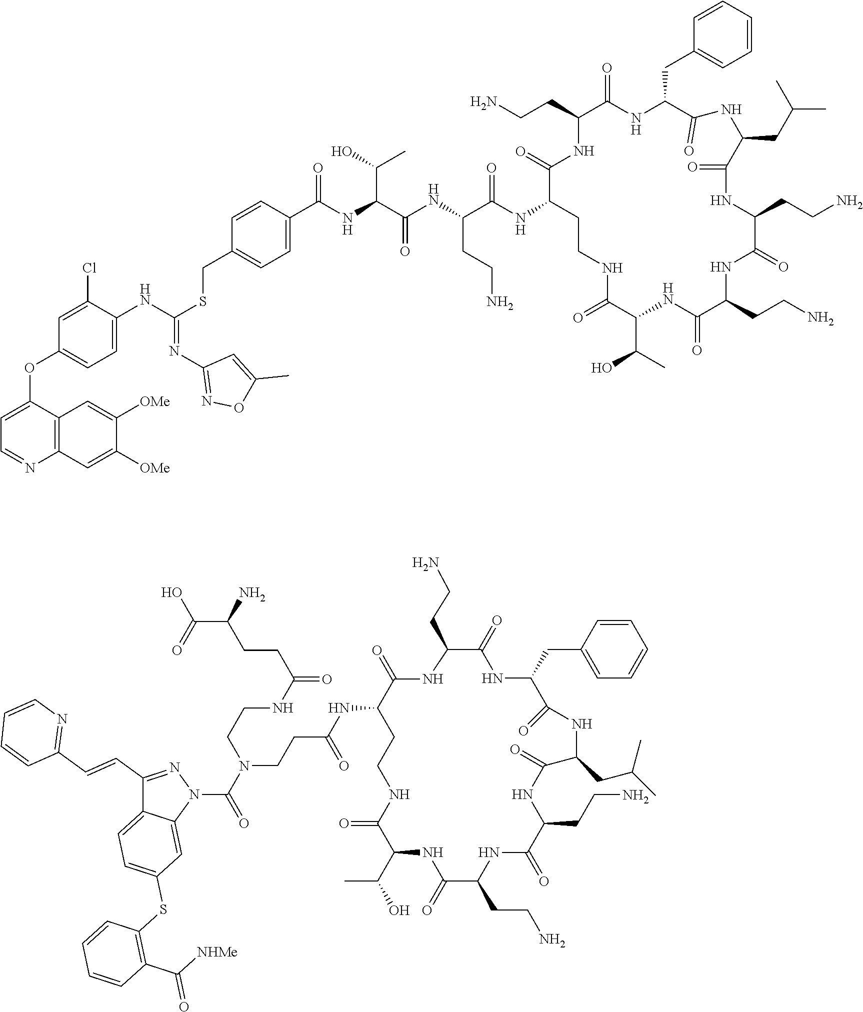 [SMILES: COc1cc2nccc(Oc3ccc(N/C(=N/c4cc(C)on4)SCc4ccc(C(=O)N[C@H](C(=O)N[C@@H](CCN)C(=O)N[C@H]5CCNC(=O)[C@@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@@H](C)O)cc4)c(Cl)c3)c2cc1OC ; CNC(=O)c1ccccc1Sc1ccc2c(/C=C/c3ccccn3)nn(C(=O)N(CCNC(=O)CC[C@H](N)C(=O)O)CCC(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)c2c1]

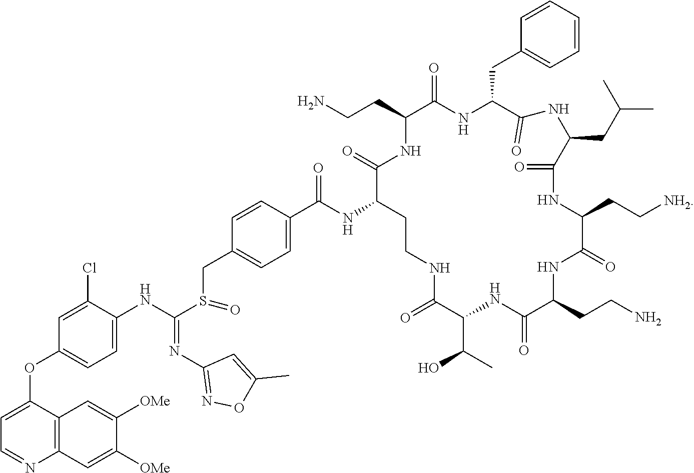 [SMILES: COc1cc2nccc(Oc3ccc(N/C(=N/c4cc(C)on4)S(=O)Cc4ccc(C(=O)N[C@H]5CCNC(=O)[C@@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)cc4)c(Cl)c3)c2cc1OC]

In one preferred embodiment, the compound is selected from the structures below, or a pharmaceutically acceptable salt, solvate, or hydrate thereof:

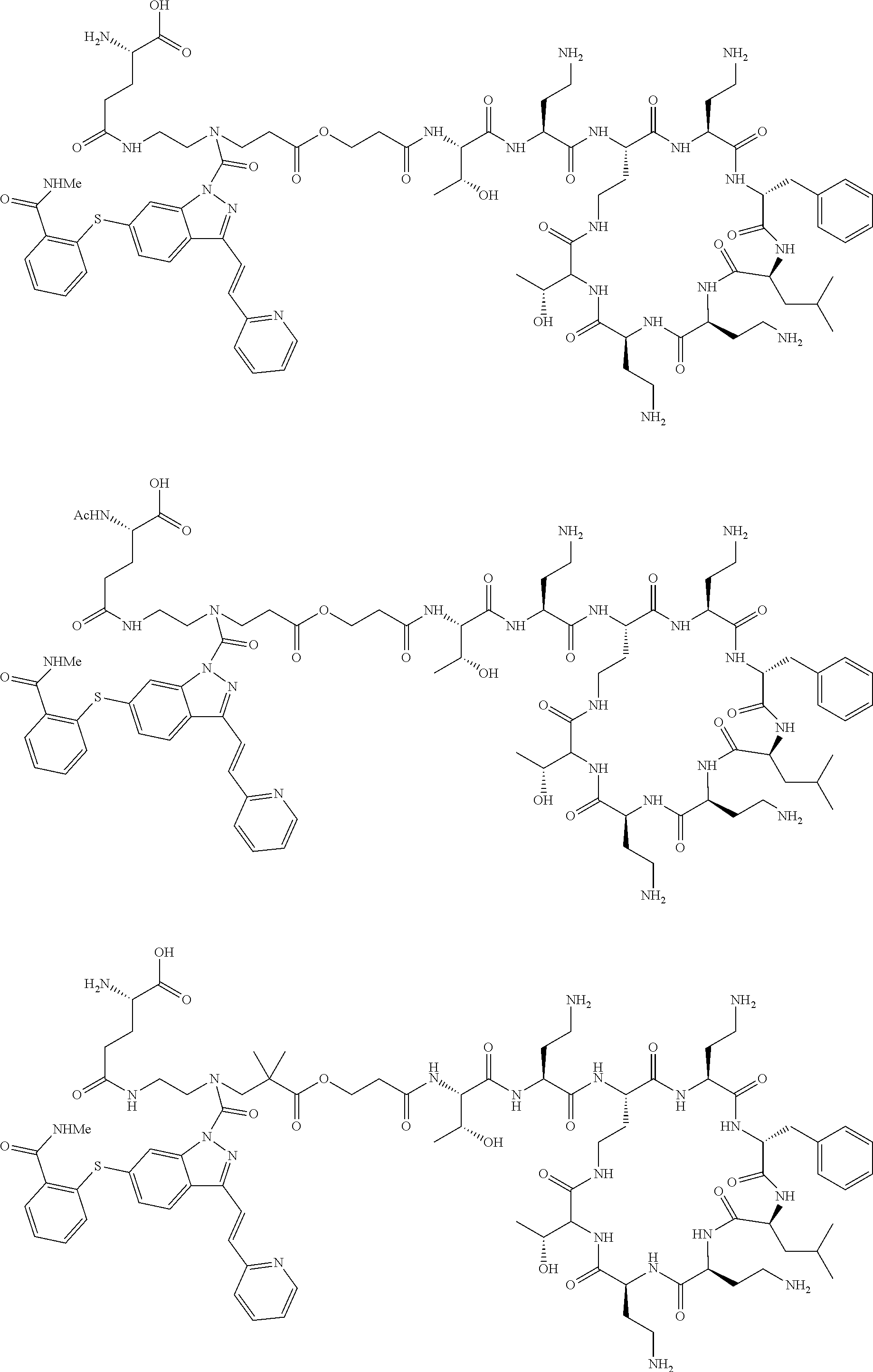

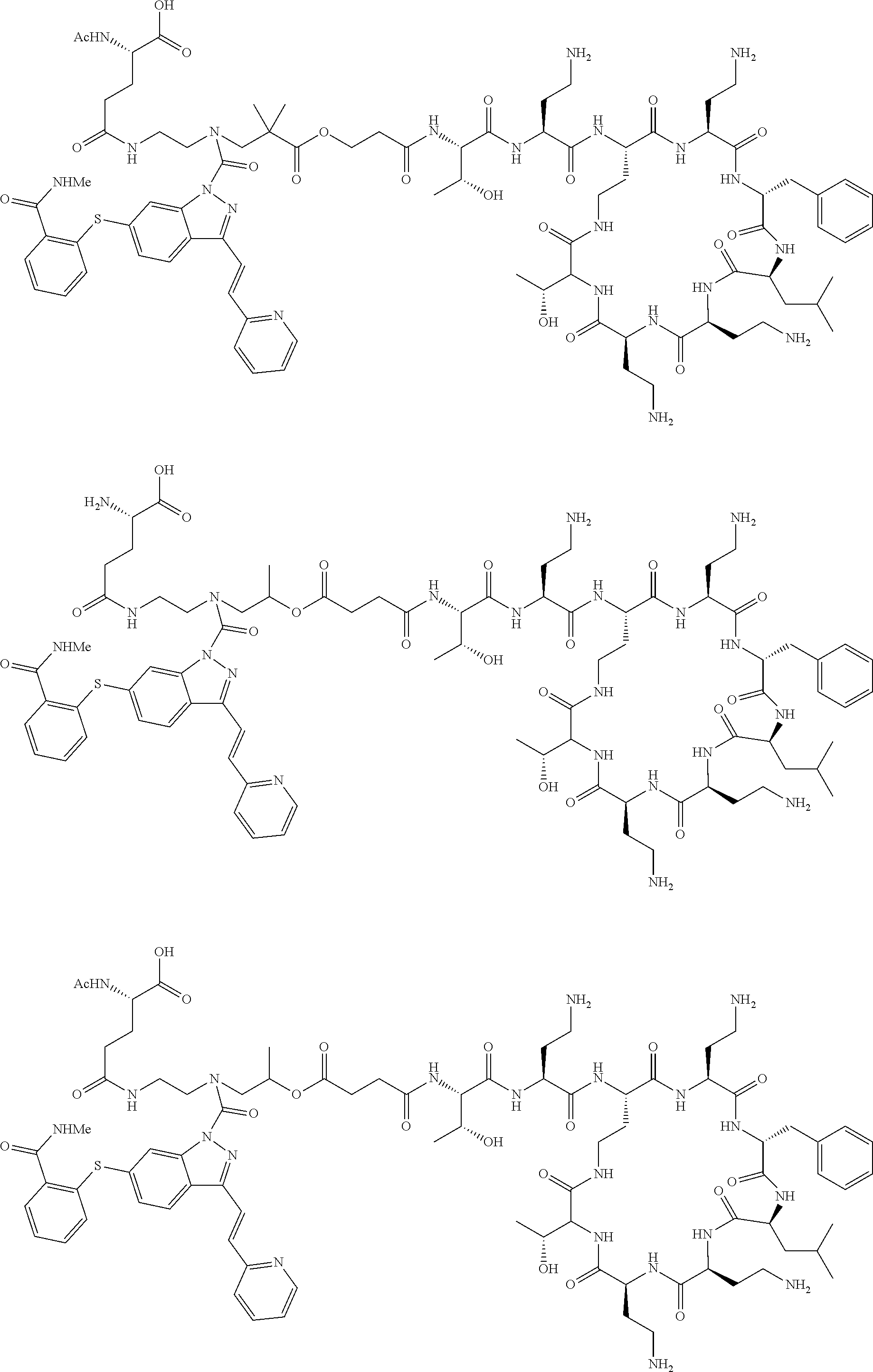

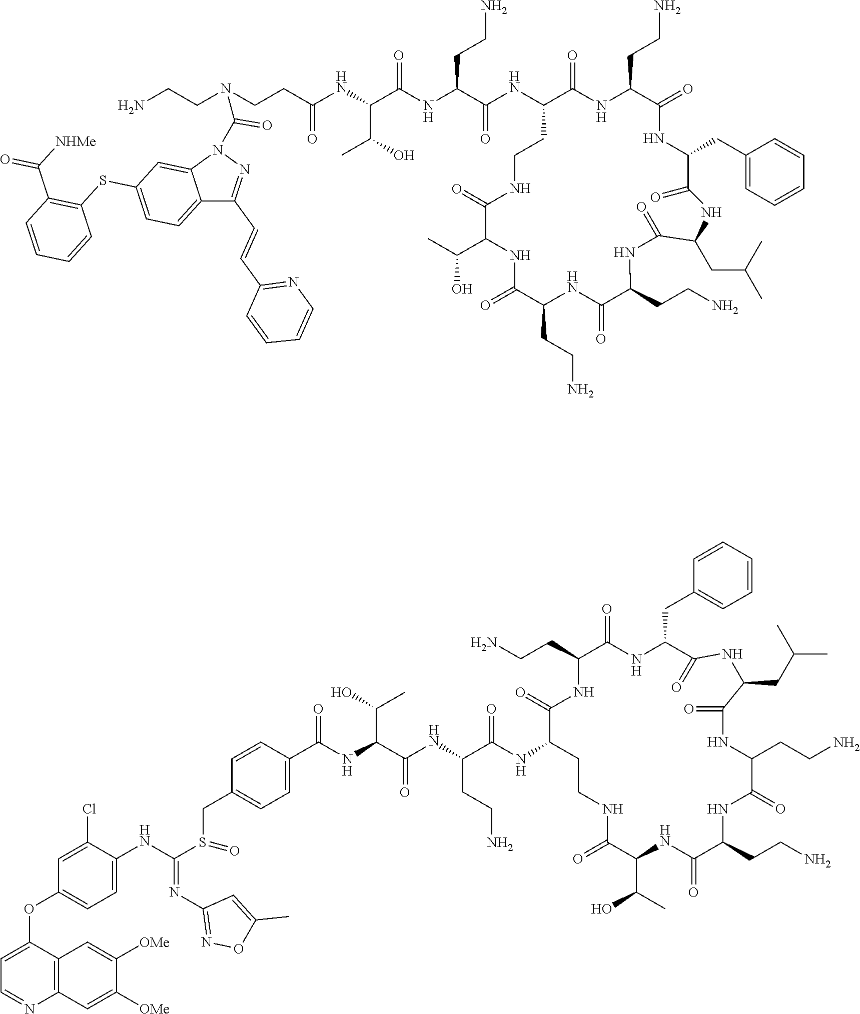 [SMILES: CNC(=O)c1ccccc1Sc1ccc2c(/C=C/c3ccccn3)nn(C(=O)N(CCN)CCC(=O)N[C@H](C(=O)N[C@@H](CCN)C(=O)N[C@H]3CCNC(=O)C([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1 ; COc1cc2nccc(Oc3ccc(N/C(=N/c4cc(C)on4)S(=O)Cc4ccc(C(=O)N[C@H](C(=O)N[C@@H](CCN)C(=O)N[C@H]5CCNC(=O)[C@H]([C@@H](C)O)NC(=O)[C@H](CCN)NC(=O)C(CCN)NC(=O)[C@H](CC(C)C)NC(=O)[C@@H](Cc6ccccc6)NC(=O)[C@H](CCN)NC5=O)[C@@H](C)O)cc4)c(Cl)c3)c2cc1OC]

In some or any embodiments the compound is according to any one of formula I-P, II-P, III-P, and I-V, of any one of embodiments 2-8, or of any embodiments as provided herein, when administered to a mammal, exhibits preferential accumulation in kidneys, with a ratio for its concentration in kidneys compared to that in blood of at least 20.

In some or any embodiments the compound is according to any one of formula I-P, II-P, III-P, and I-V, of any one of embodiments 2-8, or of any embodiments as provided herein, when administered to a mammal at a dosing (expressed in molar amount) equal to a standard therapeutic dosing (molar amount) of an agent (H)ₙR¹ and/or (H)ₒR² (or anticancer drug, or a cytotoxic compound) exhibits about 1.5- to 15-fold higher loading (tissue concentration) of agent (bioactive component) (H)ₙR¹ and/or (H)ₒR² in kidneys, as compared to the standard therapeutic dosing of (H)ₙR¹ and/or (H)ₒR² (or anticancer drug, or a cytotoxic compound).

In some or any embodiments the compound is according to any one of formula I-P, II-P, III-P, and I-V, of any one of embodiments 2-8, or of any embodiments as provided herein, when administered to a mammal at a dosing (expressed in molar amount) equal to a standard therapeutic dosing (molar amount) of an agent (H)ₙR¹ and/or (H)ₒR² (or anticancer drug, or a cytotoxic compound), exhibits at least 2-fold higher loading (tissue concentration) of a bioactive component (H)ₙR¹ and/or (H)ₒR² in kidneys, as compared to the standard therapeutic dosing of (H)ₙR¹ and/or (H)ₒR² (or anticancer drug, or a cytotoxic compound).

In some or any embodiments the compound is according to any one of formula I-P, II-P, III-P, and I-V, of any one of embodiments 2-8, or of any embodiments as provided herein, when administered to a mammal at a dosing (expressed in molar amount) equal to a standard therapeutic dosing (molar amount) of an agent (H)ₙR¹ and/or (H)ₒR² (or anticancer drug, or a cytotoxic compound likewise incorporated into a compound is according to any one of formula I-P, II-P, III-P, and I-V, of any one of embodiments 2-8, or of any embodiments as provided herein), exhibits about 1.5- to 15-fold higher efficacy, as compared to the standard therapeutic dosing of (H)ₙR¹ and/or (H)ₒR² (or anticancer drug, or a cytotoxic compound), with said therapeutic effect determined as a slowed, stopped, or reversed progression of cancer (for example, determined per changes in a cancer tumor size, and/or by using biochemical biomarkers for cancer monitoring).

In some or any embodiments the compound is according to any one of formulas I-P, II-P, III-P, and I-V, of any one of embodiments 2-8, or of any embodiments as provided herein, when administered to a mammal at a dosing (expressed in molar amount) equal to a standard therapeutic dosing (molar amount) of an agent (H)ₙR¹ and/or (H)ₒR² (or anticancer drug, or a cytotoxic compound likewise incorporated into a compound of formula I-P, II-P, III-P, and I-V, of any one of embodiments 2-8, or of any embodiments as provided herein), exhibits at least 2-fold higher efficacy, as compared to the standard therapeutic dosing of (H)ₙR¹ and/or (H)ₒR² (or anticancer drug, or a cytotoxic compound), with said therapeutic effect determined as a slowed, stopped, or reversed progression of cancer (for example, determined per changes in a cancer tumor size, and/or by using biochemical biomarkers for cancer monitoring, or similar methods).

In some or any embodiments the compound is according to any one of formula I-P, II-P, III-P, and I-V, of any one of embodiments 2-8, or of any embodiments as provided herein, when administered to a mammal at a dosing (expressed in molar amount) equal to a standard therapeutic dosing (molar amount) of an agent (H)ₙR¹ and/or (H)ₒR² (or anticancer drug, or a cytotoxic compound likewise incorporated into a compound of formula I-P, II-P, III-P, and I-V, of any one of embodiments 2-8, or of any embodiments as provided herein), exhibits at least 2-fold reduced rate (frequency or incidence) of adverse effects and/or off-target toxicity manifestation, as compared to the standard therapeutic dosing of (H)ₙR¹ and/or (H)ₒR² (or anticancer drug, or a cytotoxic compound), as determined by gross observations of a mammal under therapy, a blood cells count, a tissue biopsy, and/or by analysis of biochemical biomarkers.

In some or any embodiments, provided is a method for the treatment of a cancer disease, such as kidney or renal cancer disease, in a mammal comprising administering to the mammal a therapeutically effective amount of a compound is according to any one of formula I-P, II-P, III-P, and I-V, of any one of embodiments 2-8, or as defined in any of the embodiments described herein. In some or any embodiments, provided is a method for the treatment of a cancer disease in a mammal comprising administering to the mammal a therapeutically effective amount of a compound is according to any one of formula I-P, II-P, III-P, and I-V, of any one of embodiments 2-8, or as defined in any of the embodiments described herein wherein the compound is administered to the mammal orally, parenterally, transdermally, topically, rectally, intranasally, or by intra-tumoral administration (such as injection) in a pharmaceutical composition, including an aerosol form. In some or any embodiments, the method is that wherein the cancer is RCC or mRCC diseases.

In some embodiments and aspects, a compound provided herein may be used in a combination with an adjunct agent, to act synergistically and/or enhance therapeutic effects of said compound itself, or of an adjunct agent, or both. Such adjunct agents include other anticancer or immunomodulating agent(s), such as a monoclonal antibody agent, or another cytotoxic agent(s), or another oncology (cancer) agent, or humanized antibody such as pembrolizimab.

Such combinations of the compounds provided herein are useful for the prevention, treatment, and alleviation of symptoms of cancer diseases, in particular, kidney cancers.

In one such aspect, a compound provided herein has modest or no anticancer activity in vitro, but exhibits high anticancer efficacy when administered to a mammal in need of a cancer therapy.

In some or any embodiments is provided a pharmaceutical composition comprising a therapeutically effective amount of a compound of a compound is according to any one of formulas I-P, II-P, III-P, and I-V, of any one of embodiments 2-8, or as defined in any of the embodiments described herein, and a pharmaceutically acceptable carrier.

In another aspect, provided is a method treating a cancer in a mammal comprising administering to the mammal a therapeutically effective amount of a compound according to any one of formula I-P, II-P, III-P, and I-V, of any one of embodiments 2-8, or as defined in any of the embodiments described herein or a pharmaceutical composition thereof (i.e. the compound and a pharmaceutically acceptable carrier). In some or any embodiments, the compound is administered to the mammal parenterally, transdermally, orally, intranasally, topically, rectally, or via an intra-tumoral administration, optionally, in a pharmaceutical composition. In some or any embodiments, the cancer is renal cancer, including renal cell carcinoma (RCC) and metastatic RCC (mRCC).

### General Synthetic Methods

The compounds of this invention can be prepared in accordance with one or more of methods described, for example, in references below. General syntheses of certain related starting materials have been described in the literature. For example, the preparation of Boc-protected polymyxin nonapeptide was described by O'Dowd et al. in Tetrahedron Lett. 2007, vol. 48, p. 2003. Additional protected polymyxin B nonapeptide and colistin nonapeptide derivatives can be prepared as described by Okimura et al. in Chem. Pharm. Bull. 2007, vol. 55, pp. 1724-1730. Likewise, the general peptide acylation chemistry was described in the ref. Tetrahedron Lett. 2007, vol. 48, pp. 2003-2005.

Additional general methods suitable for preparation of compounds of formulas I-V have been described in publications WO 2016/083531, WO 2015/149131, WO 2015/135976, US 2015/0031602, WO 2014/188178, WO 2014/108469, CN 103923190, US 2014/0162937, WO 2014/028087, WO 2013/112548, CN 103130876, WO 2013/072695, WO 2012/168820, WO 2012051663, US 2012/0316105, US 2012/0283176, US 2010/0160215, US 2009/0215677, WO 2008/017734, WO 2006/045156, US 2006/0004185, U.S. Pat. Nos. 6,380,356, and 3,450,687.

Methods suitable for incorporation of suitable enzymatically and/or chemically cleavable groups X, Y, and Z (and additional spacers/linkers in same) in compounds of formulas I-V have been described in generally related synthetic art for preparation of ADCs and other agents, such as reported, for example, in publications US 20170355769; J. Am. Chem. Soc. 2018, vol. 140, p. 1617; Bioconjugate Chem. 2016, vol. 27, p. 1606; Bioconjugate Chem. 2016, vol. 27, p. 1645; Bioconjugate Chem. 2015, vol. 26, p. 919; Mol. Pharmaceutics 2015, vol. 12, p. 1813; ACS Med. Chem. Lett. 2017, vol. 8, p. 1037; ACS Med. Chem. Lett. 2016, vol. 7, p. 983; Org. Process Res. Dev. 2019, vol. 23, p. 2647; Bioconjugate Chem. 2016, vol. 27, p. 1880; Bioconjugate Chem. 2017, vol. 28, p. 620; Org. Process Res. Dev. 2018, vol. 22, p. 286; Bioconjugate Chem. 2015, vol. 26, p. 2216; J. Med. Chem. 2014, vol. 57, p. 6949; Bioconjugate Chem. 2018, vol. 29, p. 1155; J. Am. Chem. Soc. 2015, vol. 137, p. 3229; Mol. Pharmaceutics 2018, vol. 15, p. 2384; ACS Med. Chem. Lett. 2016, vol. 7, p. 988; Chem. Biodiversity 2019, vol. 16, e1800520; Nature Commun. 2018, vol. 9, p. 2512; Mol. Pharmaceutics 2011, vol. 8, p. 901; ACS Med. Chem. Lett. 2019, vol. 10, p. 1393; J. Nat. Prod. 2017, vol. 80, p. 2447; ACS Med. Chem. Lett. 2019, vol. 10, p. 1674; Pharmaceutics 2013, vol. 5, p. 220; and other references cited in these publications.

Specific methods, amino acid reagents, as well as linker/spacer structures described in above literature are directly adaptable to prepare compounds of formulas I-V, by straightforward variations in specific reagents and protection/deprotection schemes, obvious to one skilled in synthetic organic chemistry.

Additional syntheses of specific compounds described herein are illustrated by various synthetic Schemes for Examples below, likewise adaptable to preparation of additional compounds provided herein.

### Examples

Embodiments of the present invention are described in the following examples, which are meant to illustrate and not limit the scope of this invention. Common abbreviations well-known to those with ordinary skills in the synthetic art. used throughout. NMR means 400 MHz ¹H NMR spectra (delta, ppm) recorded in D₂O unless specified otherwise. LCMS means liquid chromatography mass-spectroscopy analysis. MS means mass-spectroscopy data (m/z) for a positive ionization method. Chromatography means silica gel chromatography using common organic solvents unless specified otherwise. TLC means thin-layer chromatography. HPLC means reverse-phase high-performance chromatography using commercial C18 phase columns. TES means Et₃SiH, TFA means CF₃COOH, EA means EtOAc, ACN means MeCN, DMF means N,N-dimethylformamide, DCC means N,N′-dicyclohexylcarbodiimide, DCE means 1,2-dichloroethane, NMP means N-methyl pyrrolidinone, PE means hexanes or light petroleum ether. C18 chromatography means reverse phase chromatography using a gradient of water and acetonitrile (ACN), or of same and containing 0.05% to 1% of TFA. The reagent PMBN(Boc)₄ is H-Thr-Dab(Boc)-cyclo[Dab(Boc)-Dab(Boc)-D-Phe-Leu-Dab(Boc)-Dab(Boc)-Thr]. The reagent Dab(Boc)PMBN(Boc)₄ [same as Dab(Boc)-PMBN(Boc)₄] is H-Dab(Boc)-Thr-Dab(Boc)-cyclo[Dab(Boc)-Dab(Boc)-D-Phe-Leu-Dab(Boc)-Dab(Boc)-Thr] {same as Dab(Boc)-Thr-Dab(Boc)-cyclo[Dab(Bofc)-Dab(Boc)-D-Phe-Leu-Dab(Boc)-Dab(Boc)-Thr]}. Other reagent abbreviations are just as employed in common synthetic literature, including the American Chemical Society list of abbreviations, such as found, for example, in the Journal of Organic Chemistry; or in the Journal of Peptide Chemistry. Unless specified otherwise, all reagents were either from commercial sources, or made by conventional methods described in available literature.

### Example 1

### Synthesis of the Compound of Example 1

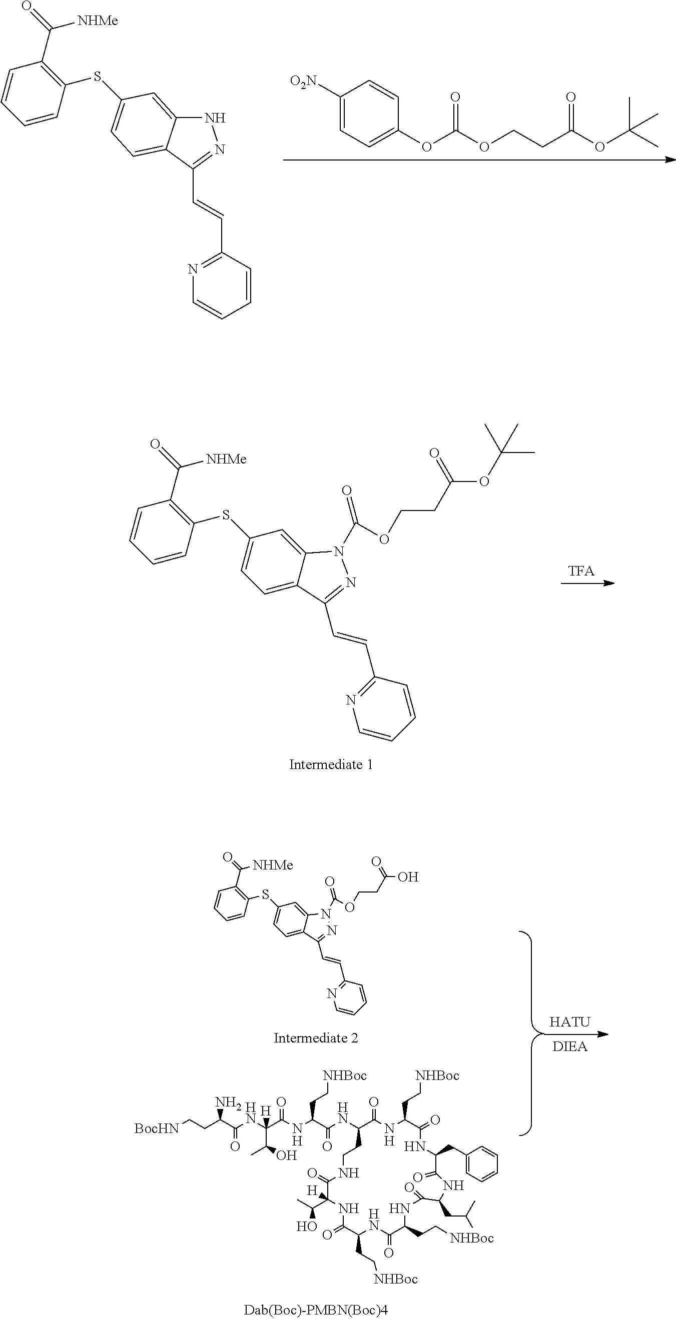

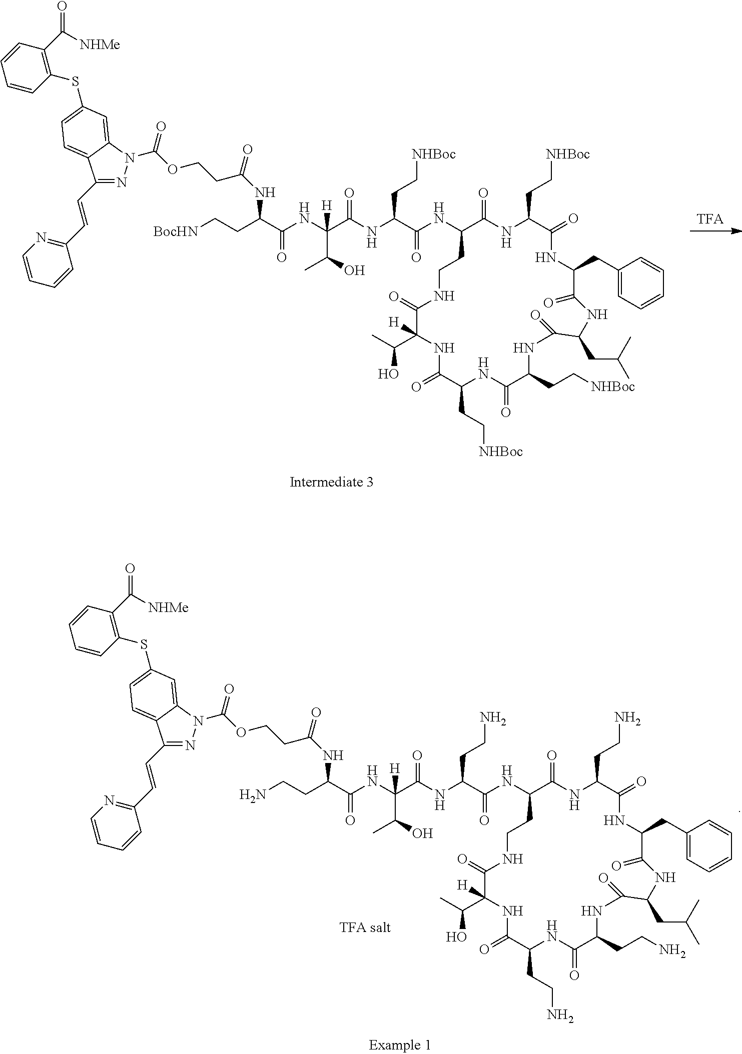

Intermediate 1. A mixture of t-butyl 3-(((4-nitrophenoxy)carbonyl)oxy)propanoate (268 mg, 0.42 mmol), (E)-N-methyl-2-((3-(2-(pyridin-2-yl)vinyl)-1H-indazol-6-yl)thio)benzamide (same as axitinib; 200 mg, 0.42 mmol), DIEA (155 μL, 0.84 mmol), DMAP (5 mg, 0.04 mmol) in 8 mL of DMF was stirred at r.t. for 36 h. The mixture was diluted with 100 mL of EA, and then washed with H₂O (10 mL×2), and brine (10 mL×2). The EA layer was dried and evaporated. The crude product was purified by HPLC to give 83 mg of Intermediate 1.

Intermediate 2. A mixture of Intermediate 1 (75 mg, 0.15 mmol) in 1 mL/1 mL of TFA/DCM was stirred at r.t. for 4 h, and evaporated to give 78 mg of Intermediate 2, which was used directly to the next step.

Intermediate 3. A mixture of Intermediate 2 (50 mg, 0.1 mmol), Dab(Boc)-PMBN(Boc)₄ (156 mg, 0.1 mmol; prepared as described in PCT WO2016100578), TEA (40 μL, 0.15 mmol), HATU (38 mg, 0.12 mmol) in 5 mL of THF was stirred at r.t. for 12 h. The reaction mixture was diluted with 100 mL of EA, and then washed with H₂O (10 mL×2), and brine (10 mL×2). The EA layer was dried and evaporated. The crude product was purified by HPLC to give 87 mg of Intermediate 3.

Compound of Example 1. A mixture Intermediate 3 (87 mg, 0.05 mmol) in 0.5 mL/2 mL of TFA/DCM was stirred at r.t. for 3 h. Volatiles were evaporated, the crude product was purified by HPLC to give 53 mg of the Compound of Example 1. NMR: 8.56 (d, J 3.2 Hz, 1H), 8.37 (t, J 5.6 Hz, 1H), 8.15 (d, J 5.6 Hz, 1H), 7.79-7.69 (m, 3H), 7.55-7.35 (m, 5H), 7.31-7.11 (m, 7H), 4.48-4.36 (m, 5H), 4.22-4.01 (m, 9H), 3.23-3.19 (m, 1H), 3.06-2.57 (m, 20H), 2.17-1.73 (m, 14H), 1.41-1.37 (m, 2H), 1.07 (dd, J 7.6, 3.6 Hz, 3H), 0.99-0.94 (m, 3H), 0.71-0.57 (m, 8H). MS: 1548.5 [M+H]⁺.

### Example 2

### Synthesis of the Compound of Example 2

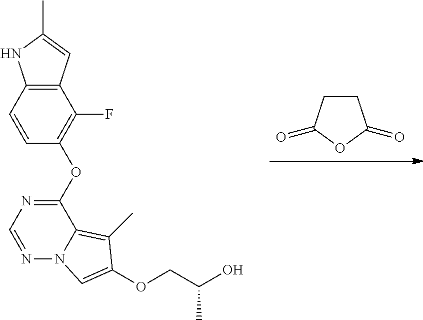 [SMILES: Cc1cc2c(F)c(Oc3ncnn4cc(OC[C@@H](C)O)c(C)c34)ccc2[nH]1 ; O=C1CCC(=O)O1]

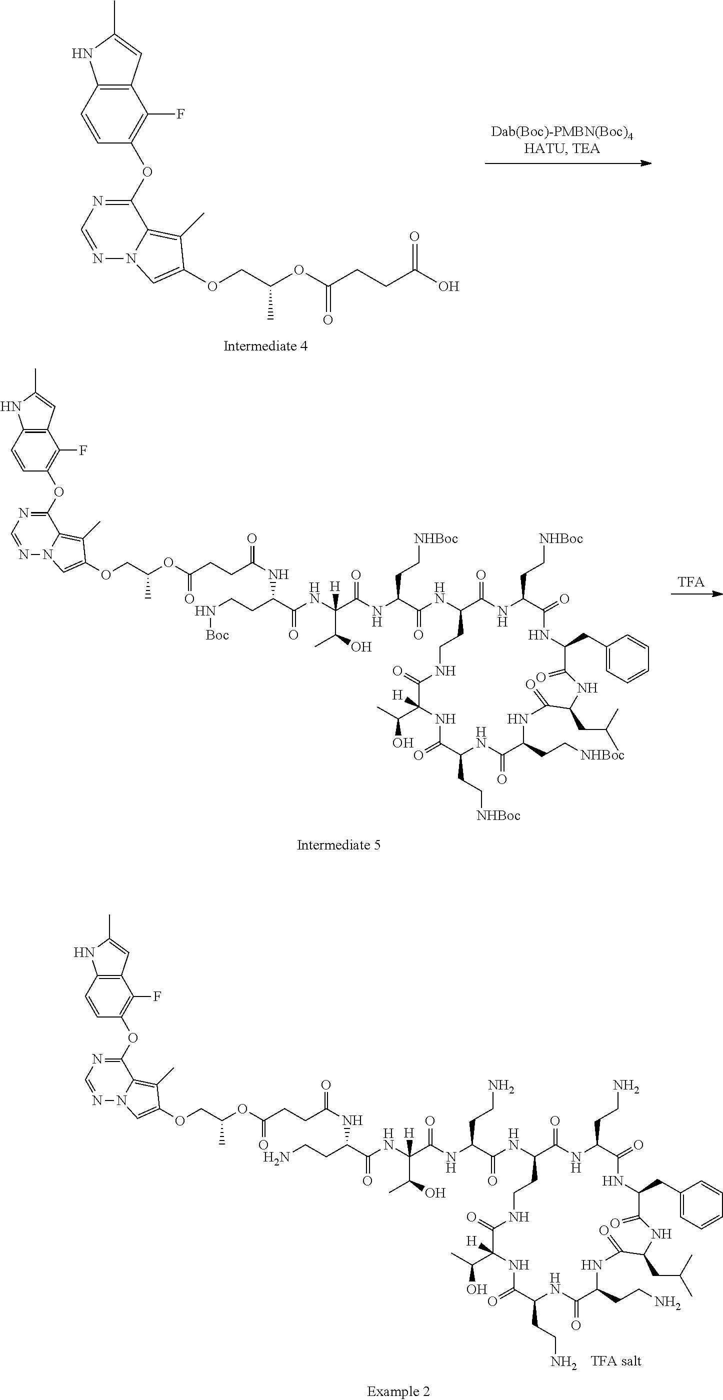

Intermediate 4. A mixture of (R)-1-((4-((4-fluoro-2-methyl-1H-indol-5-yl)oxy)-5-methylpyrrolo[2,1-f][1,2,4]triazin-6-yl)oxy)propan-2-ol (50 mg, 0.13 mmol), succinic anhydride (39 mg, 0.39 mmol) in Py (0.5 mL) was stirred at 80° C. for 24 h. The reaction mixture was diluted with 50 mL of EA, and then washed with H₂O (10 mL×2), brine (10 mL×2), dried and evaporated. The crude product was purified by silica gel chromatography to give 58 mg of Intermediate 4.

Intermediate 5. A mixture of Intermediate 4 (50 mg, 0.1 mmol), Dab(Boc)-PMBN(Boc)₄ (156 mg, 0.1 mmol), DIEA (36 μL, 0.20 mmol), HATU (39 mg, 0.12 mmol) in 10 mL of THF was stirred at r.t. for 16 h. The reaction mixture was diluted with 100 mL of EA, and then washed with H₂O (10 mL×2), and brine (10 mL×2), The EA layer was dried and evaporated. The crude product was purified by HPLC to give 112.6 mg of Intermediate 5.

Compound of Example 2. A mixture of Intermediate 5 (150 mg, 0.07 mmol) in 2 mL/8 mL of TFA/DCM was stirred at r.t. for 3 h, then evaporated, and the crude product was purified by HPLC to give 86 mg of the Compound of Example 2. NMR: 7.49 (s, 1H), 7.20-7.02 (m, 6H), 6.76-6.68 (m, 2H), 4.93 (s, 1H), 4.42-4.37 (m, 4H), 4.26-4.07 (m, 8H), 3.68 (bds, 2H), 3.28-3.25 (m, 1H), 3.06-2.73 (m, 13H), 2.35-1.76 (m, 22H), 1.36-1.22 (m, 2H), 1.09-1.08 (m, 9H), 0.59 (s, 4H), 0.51 (s, 3H). MS: 1516.7 [M+H]⁺.

### Example 3

### Synthesis of the Compound of Example 3

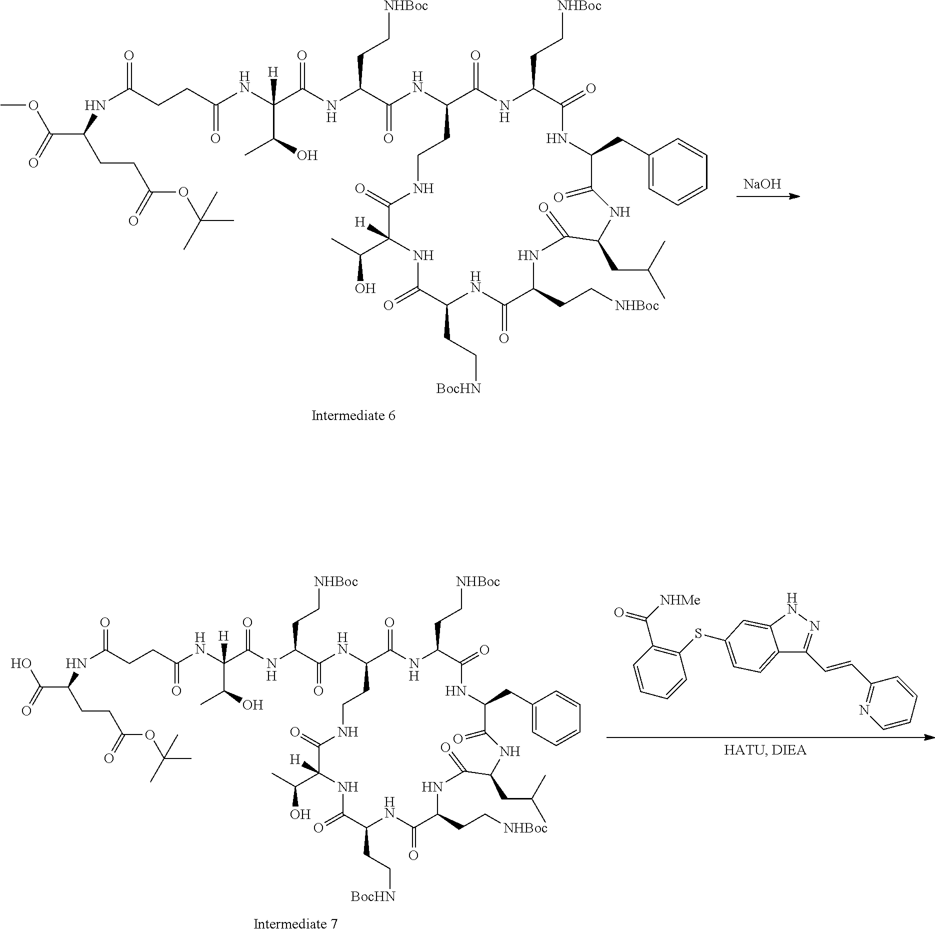

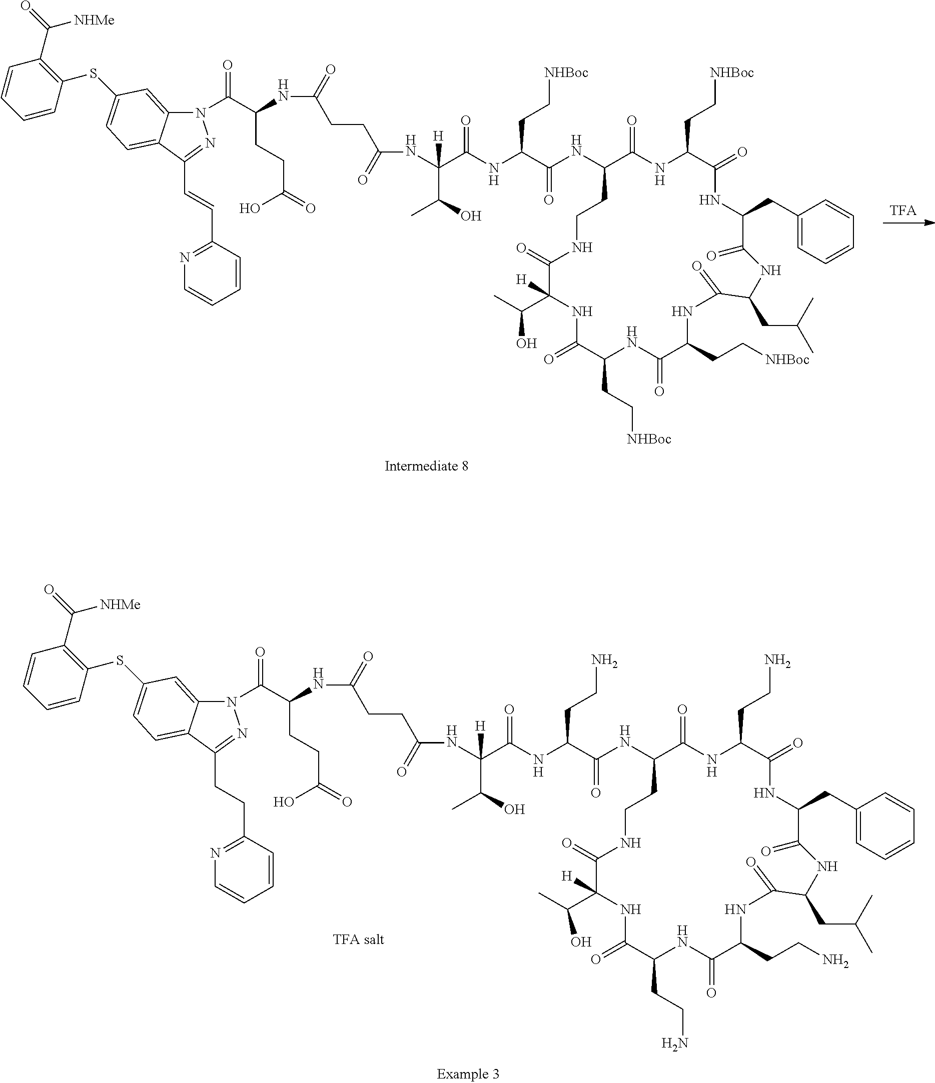

Intermediate 7. A mixture of Intermediate 6 (1.3 g, 0.8 mmol), NaOH (50 mg, 1.2 mmol) in 10 mL/10 mL of H₂O/THF was stirred at 0° C. for 1.5 h. 1 M of HCl to control pH=6.0 was added, and the reaction mixture diluted with 200 mL of EA, and then washed with H₂O (20 mL×2), brine (20 mL×2). The EA layer was dried and evaporated. The crude product was purified by HPLC to give 1.1 g of Intermediate 7.

Intermediate 8. A mixture of Intermediate 7 (330 mg, 0.21 mmol), (E)-N-methyl-2-((3-(2-(pyridin-2-yl)vinyl)-1H-indazol-6-yl)thio)benzamide (78 mg, 0.21 mmol), TEA (120 μL, 0.63 mmol), HATU (120 mg, 0.33 mmol) in 10 mL of DMF was stirred at r.t. for 6 h. The reaction mixture was diluted with 200 mL of EA, and then washed with H₂O (50 mL×2), and brine (50 mL×2). The EA layer was dried and evaporated. The crude product was purified by HPLC to give 158 mg of Intermediate 8.

Compound of Example 3. A mixture of Intermediate 8 (150 mg, 0.07 mmol) in 1 mL/3 mL of TFA/DCM was stirred at r.t. for 2 h, then volatiles were evaporated and the crude product was purified by HPLC to give 55 mg of the Compound of Example 3. NMR: 8.55-8.30 (m, 2H), 8.10-7.96 (m, 2H), 7.79-6.92 (m, 16H), 4.54-4.01 (m, 18H), 3.28-1.72 (m, 43H), 3.08-2.91 (m, 12H), 2.80-2.66 (m, 2H), 1.41-1.29 (m, 2H), 1.16-1.00 (m, 7H), 0.80-0.55 (m, 9H). MS: 1560.6 [M+H]⁺.

### Example 4

### Synthesis of the Compound of Example 4

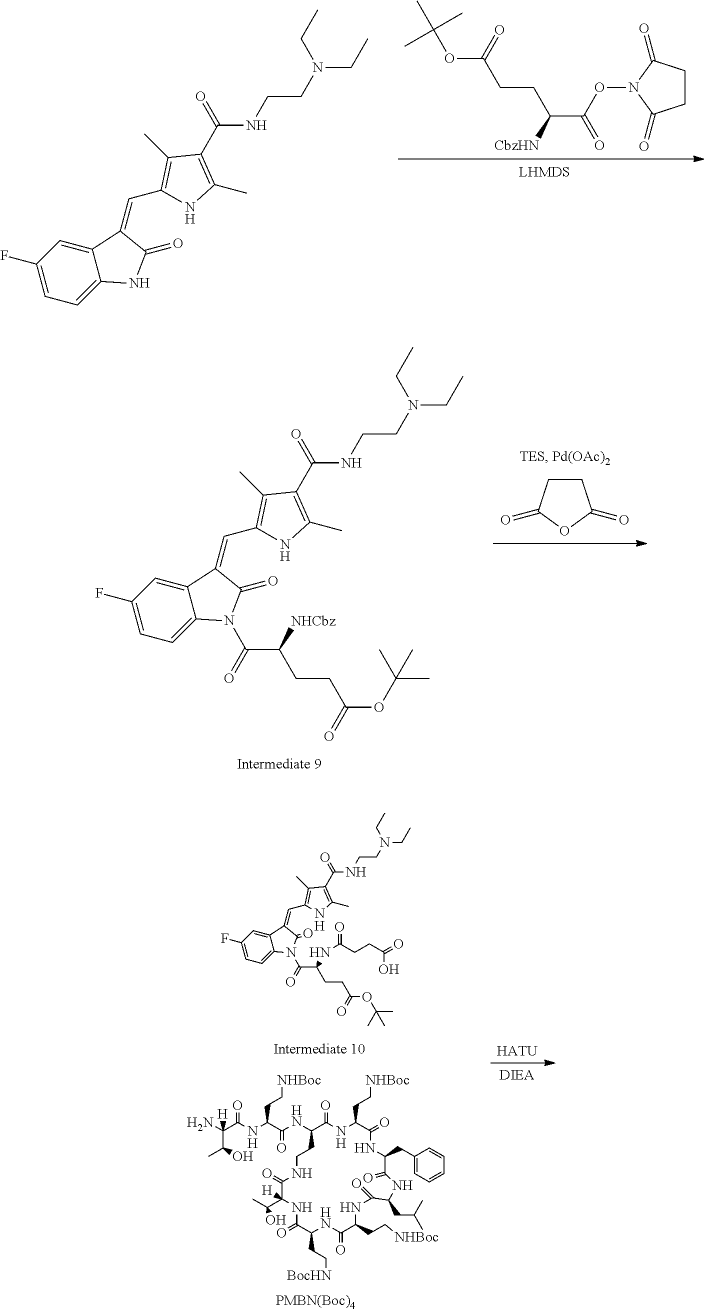

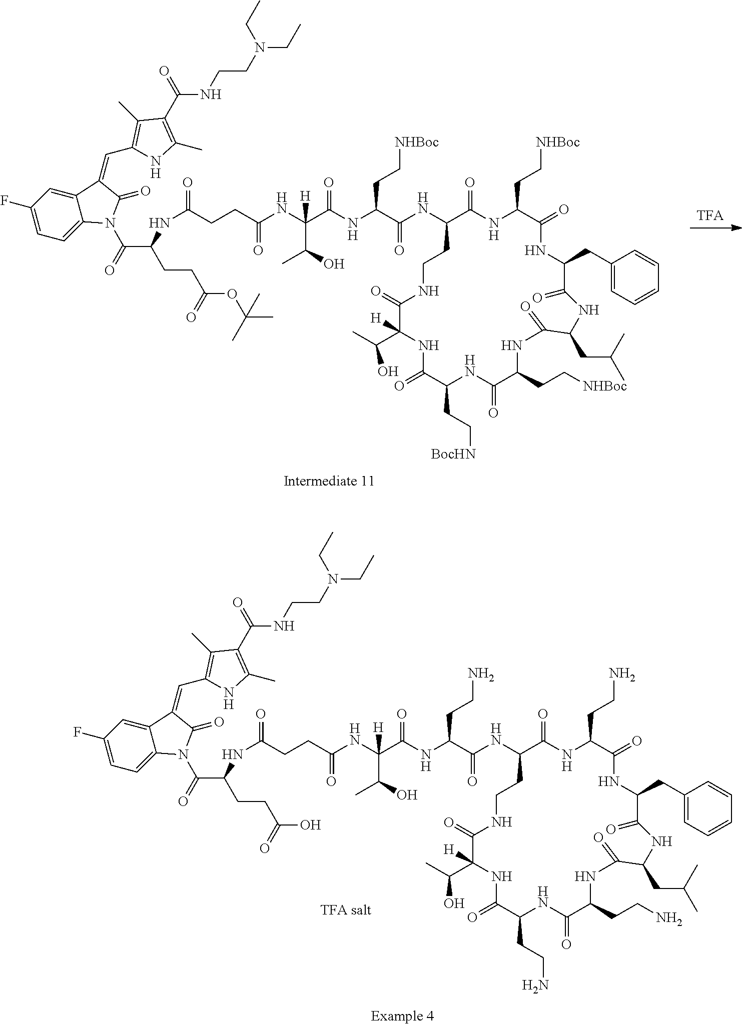

Intermediate 9. 1 M of LiHMDS (12 mL, 12 mmol) was added to (Z)—N-(2-(diethylamino)ethyl)-5-((5-fluoro-2-oxoindolin-3-ylidene)methyl)-2,4-dimethyl-1H-pyrrole-3-carboxamide (2.1 g, 4.8 mmol) in 15 mL of THF at −78° C. The mixture was stirred at −30° C. for 0.5 h. Then CbzGlu(t-Bu)OSu (5.2 g, 12 mmol) was added in one portion. The mixture was stirred at r.t. for 2 h. The reaction was quenched with NH₄Cl solution at 0° C. The reaction mixture was diluted with 250 mL of EA, and then washed with H₂O (30 mL×2), and brine (30 mL×2). The EA layer was dried and evaporated. The crude product was purified by silica gel chromatography to give 718 mg of the Intermediate 9.

Intermediate 10. A mixture of Intermediate 9 (330 mg, 0.45 mmol), succinic anhydride (90 mg, 0.9 mmol), TES (216 mg, 1.35 mmol), Pd(OAc)₂ (9 mg, 0.045 mmol), TEA (54 μL, 0.36 mmol) in 20 mL of DCM was stirred at r.t. for 2 h. Volatiles were evaporated, the residue diluted with 100 mL of EA, and then washed with H₂O (10 mL×2), and brine (10 mL×2). The EA layer was dried and evaporated. The crude product was purified by HPLC to give 148 mg of Intermediate 10.

Intermediate 11. A mixture of Intermediate 10 (158 mg, 0.23 mmol), PMBN(Boc)₄ (308 mg, 0.23 mmol), TEA (50 μL, 0.35 mmol), HATU (106 mg, 0.28 mmol) in 10 mL of THE was stirred at r.t. for 5 h. The reaction mixture was diluted with 100 mL of EA, and then washed with H₂O (10 mL×2), and brine (10 mL×2). The EA layer was dried and evaporated. The crude product was purified by HPLC to give 219 mg of Intermediate 11.

Compound of Example 4. A mixture of Intermediate 11 (116 mg, 0.03 mmol) in 0.5 mL/5 mL of TFA/DCM was stirred at r.t. for 4 h. Volatiles were evaporated, and the crude product was purified by HPLC to give 73 mg of the Compound of Example 4. NMR: 7.72-7.70 (m, 1H), 7.32-7.12 (m, 8H), 6.81-6.78 (m, 1H), 5.73-5.71 (m, 1H), 4.49 (t, J 4.8 Hz, 1H), 4.35-4.31 (m, 2H), 4.23-3.96 (m, 10H), 3.70 (t, J 4.4 Hz, 2H), 3.36-3.19 (m, 8H), 3.05-2.47 (m, 21H), 2.33 (d, J 6.4 Hz, 6H), 2.22-1.71 (m, 16H), 1.41-1.27 (m, 10H), 1.18-1.15 (m, 3H), 1.03 (d, J 4.4 Hz, 3H), 0.76-0.56 (m, 9H). MS: 1572.8 [M+H]⁺.

### Example 5

### Synthesis of the Compound of Example 5

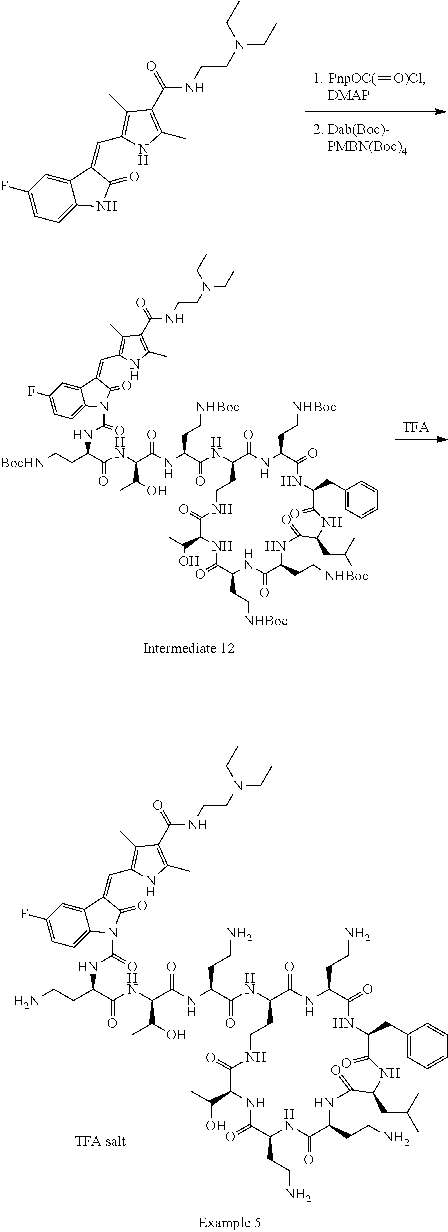

Intermediate 12. A mixture of (Z)—N-(2-(diethylamino)ethyl)-5-((5-fluoro-2-oxoindolin-3-ylidene)methyl)-2,4-dimethyl-1H-pyrrole-3-carboxamide (100 mg, 0.25 mmol), 4-nitrophenyl carbonochloridate (393 mg, 0.25 mmol) and DMAP (34 mg, 0.276 mmol) in DMF (3 mL) was stirred at r.t. for 1.5 h, then Dab(Boc)-PMBN(Boc)₄ was added to the reaction. The reaction was stirred at r.t. o.n. The mixture was extracted with EA (30 mL), washed with H₂O (5 mL×2) and brine (5 mL). The EA layer was dried and evaporated. The product was purified by silica gel chromatography (MeOH/DCM=0˜15%) to give 0.25 g of Intermediate 12.

Compound of Example 5. The mixture of Intermediate 12 (0.25 g, 0.126 mmol) in TFA/DCM (1/5 mL) was stirred at r.t for 1.5 h. Volatiles were removed from the mixture and the residue was purified by C18 chromatography (ACN/H₂O=0˜40%) to give 135 mg of the Compound of Example 5. NMR: 7.78 (s, 1H), 7.48 (s, 1H), 7.29 (ddd, J=28.3, 21.5, 8.1 Hz, 4H), 7.14 (d, J=7.3 Hz, 2H), 6.90 (t, J=9.1 Hz, 1H), 4.64 (dd, J=9.0, 5.3 Hz, 1H), 4.47 (t, J=8.2 Hz, 1H), 4.41 (q, J=6.5, 5.3 Hz, 2H), 4.24 (p, J=6.0 Hz, 1H), 4.21-4.06 (m, 6H), 3.69 (t, J=6.5 Hz, 2H), 3.33 (t, J=6.5 Hz, 2H), 3.25 (q, J=7.3 Hz, 5H), 3.13 (t, J=7.9 Hz, 2H), 3.09-2.96 (m, 8H), 2.94 (d, J=8.5 Hz, 2H), 2.70 (td, J=12.8, 11.4, 6.3 Hz, 1H), 2.41 (s, 2H), 2.32 (d, J=7.3 Hz, 1H), 2.30 (s, 3H), 2.24-1.98 (m, 8H), 1.75 (d, J=9.7 Hz, 1H), 1.38 (d, J=9.5 Hz, 1H), 1.27 (t, J=7.3 Hz, 6H), 1.19 (d, J=6.4 Hz, 3H), 1.07 (d, J=6.5 Hz, 3H), 0.66 (s, 3H), 0.58 (d, J=5.6 Hz, 3H). MS: 1487.6 [M+H]⁺.

### Example 6

### Synthesis of the Compound of Example 6

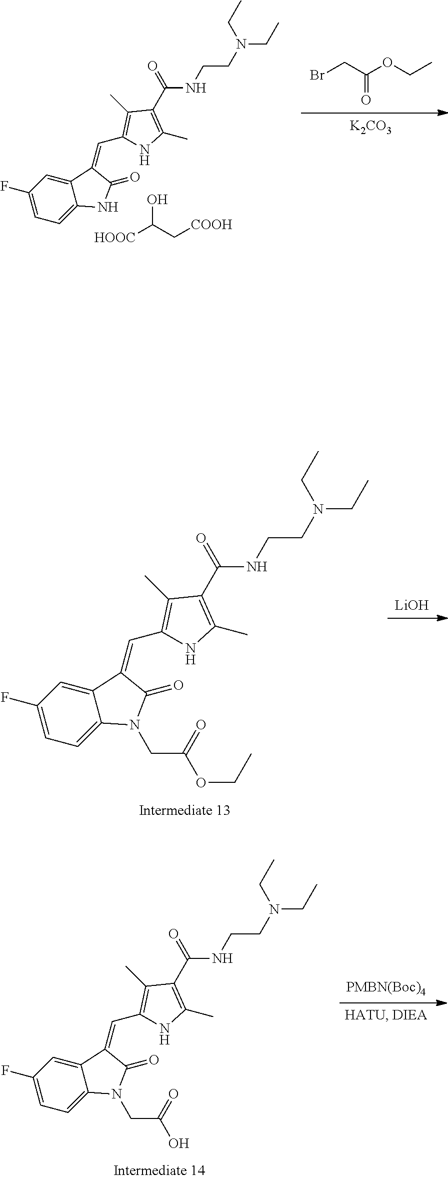

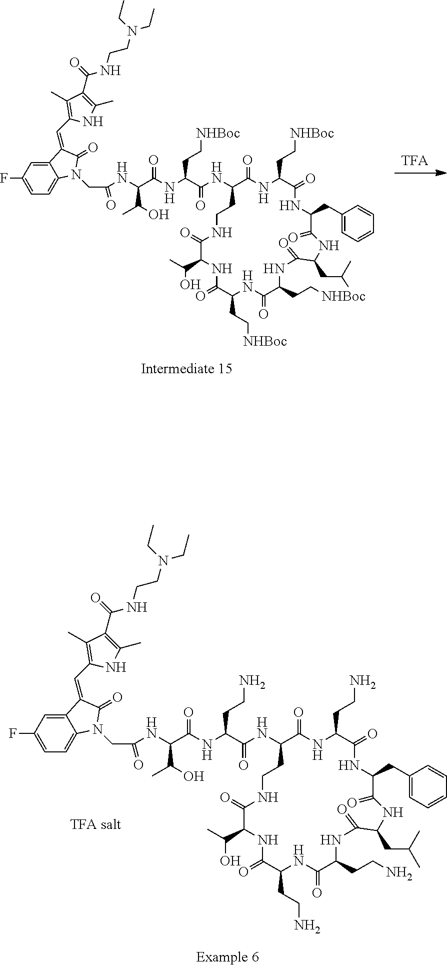

Intermediate 13. The mixture of (Z)—N-(2-(diethylamino)ethyl)-5-((5-fluoro-2-oxoindolin-3-ylidene)methyl)-2,4-dimethyl-1H-pyrrole-3-carboxamide malate (600 mg, 1.13 mmol) and K₂CO₃ (310 mg, 2.25 mmol) in DMF (10 mL) was stirred at r.t for 1.5 h, then ethyl bromoacetate was added, and stirred at r.t. about 55-60 h. The mixture was extracted with EA (100 mL), washed with H₂O (20 mL×2) and brine (15 mL). The EA layer was dried and evaporated. The product was purified by silica gel chromatography (MeOH/DCM=0˜18%) to give 0.4 g of Intermediate 13.

Intermediate 14. The mixture of Intermediate 13 (0.48 g, 1 mmol) and LiOH (126 mg, 3 mmol) in H₂O-MeOH (3/3 mL) was stirred at r.t. for 2.5 h, and pH adjusted to about 5 with aq. HCl. Volatiles were removed and the product was purified by HPLC (ACN/H₂O=0˜50%) to give 340 mg of Intermediate 14.

Intermediate 15. The mixture of Intermediate 14 (100 mg, 0.22 mmol), PMBN(Boc)₄ (299 mg, 0.22 mmol), HATU (92 mg, 0.24 mmol) and DIEA (0.078 mL, 0.44 mmol) was stirred at r.t under Ar for 2.5 hr, and the product extracted with EA (100 mL), and washed with H₂O (15 mL×2) and brine (15 mL). The EA layer was dried and evaporated, and the residue purified by silica chromatography (MeOH-DCM=0˜15%) to give 0.2 g of Intermediate 15.

Compound of Example 6. The mixture of Intermediate 15 (200 mg, 0.11 mmol) in TFA/DCM (1.5/8 mL) was stirred at r.t. for 1 h, and volatiles removed. The residue was purified by HPLC (ACN/H₂O=0˜50%) to give 210 mg of the Compound of Example 6. NMR: 7.29-7.14 (m, 5H), 7.11 (d, J=7.6 Hz, 3H), 6.80 (d, J=9.5 Hz, 1H), 6.71 (s, 1H), 4.45 (t, J=8.4 Hz, 1H), 4.23-4.17 (m, 4H), 4.12 (ddd, J=14.8, 10.3, 4.5 Hz, 2H), 4.05 (t, J=5.8 Hz, 1H), 3.96 (dd, J=9.9, 4.2 Hz, 2H), 3.67 (t, J=6.7 Hz, 2H), 3.31 (t, J=6.8 Hz, 2H), 3.25 (q, J=7.3 Hz, 5H), 3.06-2.85 (m, 11H), 2.75 (s, 1H), 2.64 (s, 2H), 2.31-2.19 (m, 7H), 2.12 (dtd, J=21.9, 14.6, 13.7, 7.8 Hz, 5H), 2.01 (dd, J=9.2, 5.3 Hz, 2H), 1.90 (dt, J=16.1, 7.2 Hz, 1H), 1.67-1.58 (m, 1H), 1.47 (s, 1H), 1.40-1.30 (m, 2H), 1.27 (t, J=7.3 Hz, 7H), 1.19 (d, J=5.8 Hz, 3H), 1.00 (d, J=6.5 Hz, 3H), 0.77 (s, 1H), 0.67 (d, J=6.4 Hz, 3H), 0.60 (d, J=6.5 Hz, 3H). MS: 1401.7 [M+H]⁺.

### Example 7

### Synthesis of the Compound of Example 7

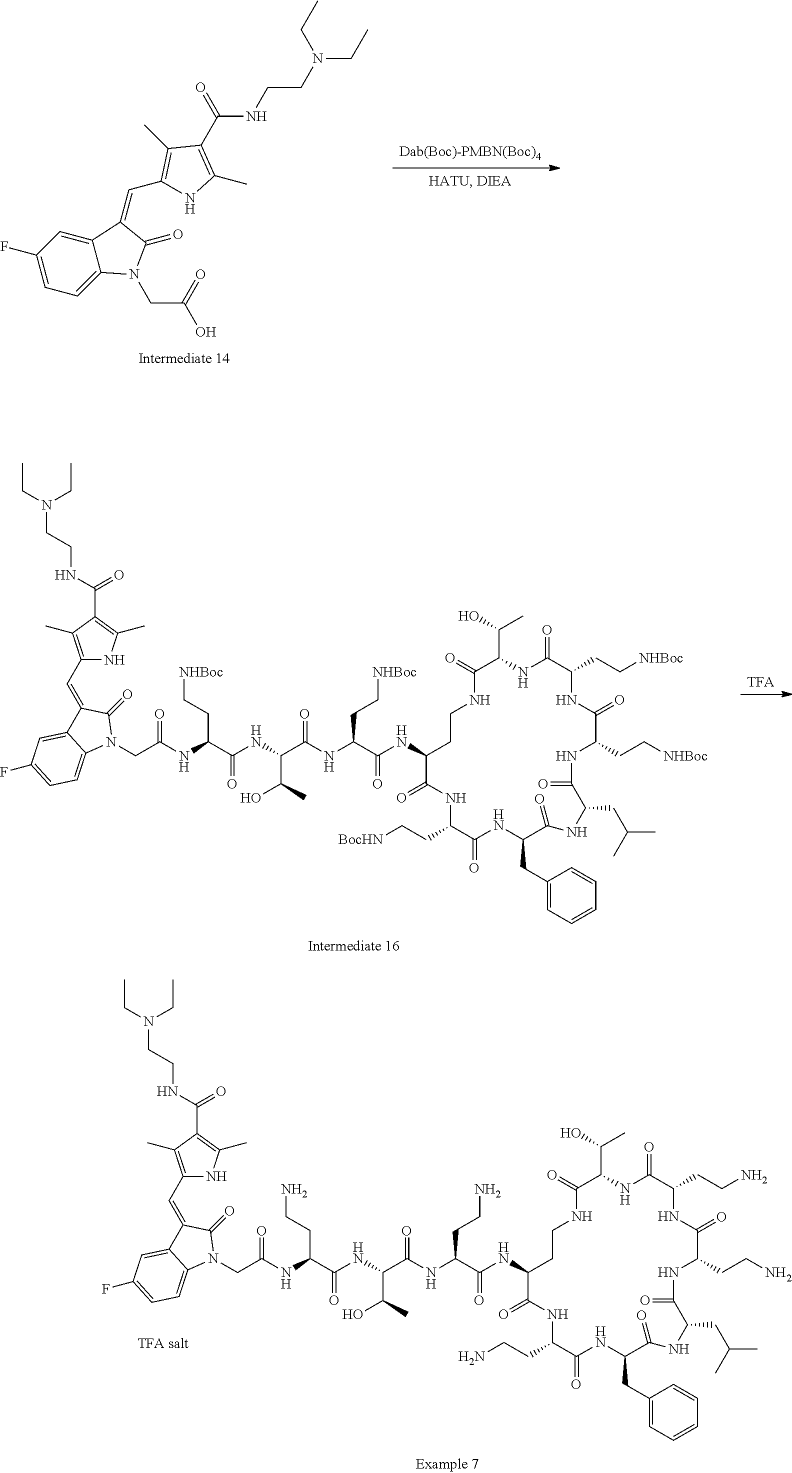

Intermediate 16. A 25 mL flask was charged with Intermediate 14 (110.0 mg, 0.241 mmol), Dab(Boc)-PMBN(Boc)₄ (414.6 mg, 0.265 mmol), HATU (100.8 mg, 0.265 mmol), DIEA (123.8 ul, 0.723 mmol) and dry DMF (2.5 mL) under Ar. The reaction mixture was stirred at r.t. for 16 h. To the reaction mixture was added water, and the product extracted three times with EA. The combined EA layer was washed with 5% NaCl twice, brine once and dried over anhydrous Na₂SO₄. The filtrate was evaporated under vacuum, dried under vacuum then purified by HPLC eluting with ACN-H₂O (0.05% TFA, 0˜100% in 50 min) to afford Intermediate 16 as a yellow solid (219.5 mg).

Compound of Example 7. A 25 mL flask was charged with Intermediate 16 (219.5 mg, 1.0 eq.), TFA (0.4 mL) and DCE (2 mL) under Ar. The reaction mixture was stirred at r.t. for 6 h. Volatiles were removed under vacuum, then dried under vacuum to give crude product. The residue was purified by HPLC eluting with ACN/Water (0.05% TFA, 0-100% in 60 min) to afford the Compound of Example 7 as a yellow solid (145.0 mg). NMR: 7.36-7.22 (m, 6H), 7.14 (d, J=4.4 Hz, 2H), 6.81 (t, J=4.0 Hz, 1H), 4.58 (d, J=12.0 Hz, 1H), 4.47˜4.44 (m, 3H), 4.36-4.34 (m, 2H), 4.23 (d, J=2.8 Hz, 1H), 4.19-4.17 (m, 2H), 4.11˜4.05 (m, 6H), 3.71 (t, J=4.0 Hz, 1H), 3.66 (t, J=4.4 Hz, 1H), 3.56 (s, 1H), 3.55-3.17 (m, 8H), 3.03˜2.92 (m, 15H), 2.75-2.66 (m, 2H), 2.26 (t, J=8.4 Hz, 5H), 2.15-2.09 (m, 9H), 1.91˜1.72 (m, 6H), 1.38-1.36 (m, 1H), 1.27˜1.24 (m, 8H), 1.06 (d, J=4.4 Hz, 3H), 1.01 (d, J=4.4 Hz, 3H), 0.65 (d, J=4.0 Hz, 3H), 0.58 (d, J=3.6 Hz, 3H). MS: 1501.7 [M+H]+.

### Example 8

### Synthesis of the Compound of Example 8

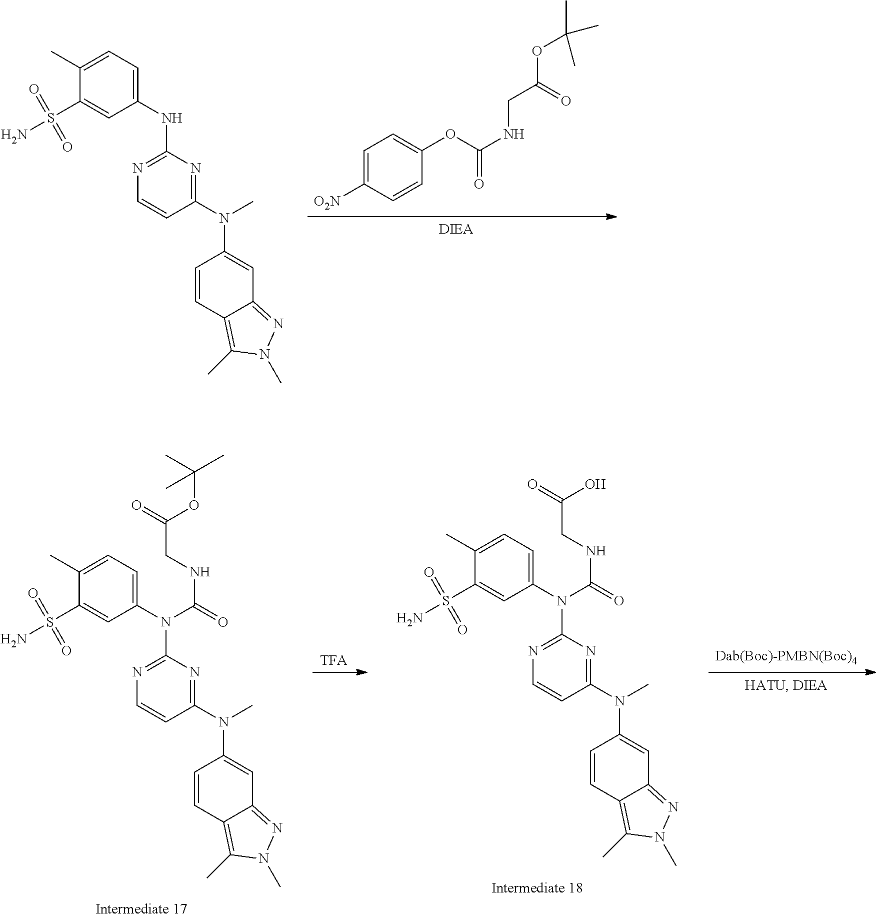

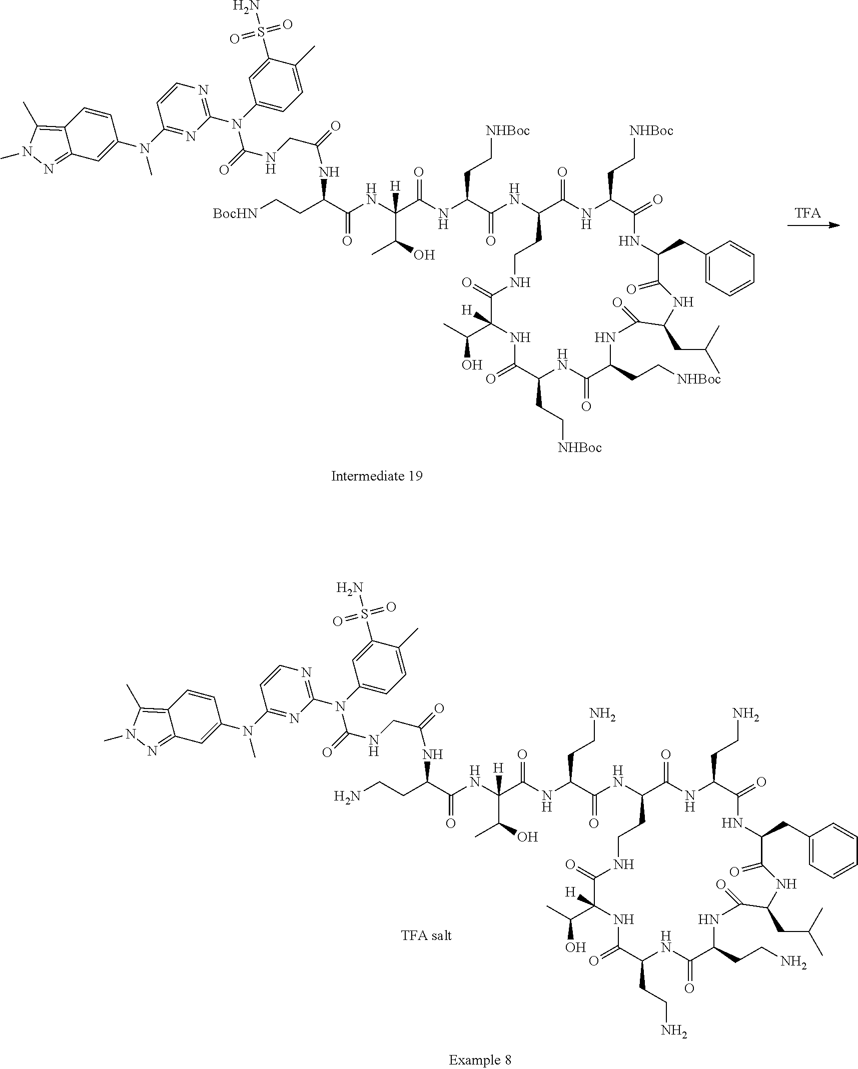

Intermediate 17. A mixture of tert-butyl 2-(((4-nitrophenoxy)carbonyl)amino)acetate (1.56 g, 5.0 mmol), 5-((4-((2,3-dimethyl-2H-indazol-6-yl)(methyl)amino)pyrimidin-2-yl)amino)-2-methylbenzenesulfonamide (438 mg, 1.0 mmol), DIEA (1.4 mL, 8.0 mmol) in 25 mL of DMF was stirred at r.t. for 16 h. The volatiles were removed by evaporation, and the residue was diluted with 200 mL of EA, and then washed with H₂O (20 mL×2), and brine (10 mL×2). The EA layer was dried and volatiles evaporated. The crude product was purified by HPLC to give 296 mg of Intermediate 17.

Intermediate 18. A mixture of Intermediate 17 (180 mg, 0.3 mmol) in 1 mL/3 mL of TFA/DCM was stirred at r.t. for 5 h. Volatiles were evaporated to give 148 mg of Intermediate 18, which was used directly.

Intermediate 19. A mixture of Intermediate 18 (75 mg, 0.14 mmol), DAB(BOC)-PMBN(BOC)₄ (226 mg, 0.14 mmol), TEA (36 mg, 0.25 mmol), HATU (65 mg, 0.17 mmol) in 10 mL of THE was stirred at r.t. for 6 h. The reaction mixture was diluted with 100 mL of EA, and then washed with H₂O (10 mL×2), brine (10 mL×2), dried and evaporated. The crude product was purified by HPLC to give 109 mg of Intermediate 19.

Compound of Example 8. A mixture of Intermediate 19 (109 mg, 0.04 mmol) in 0.5 mL/8 mL of TFA/DCM was stirred at r.t. for 5 h. Volatiles were evaporated, and the crude product was purified by HPLC to give 18 mg of the Compound of Example 8. NMR: 7.78-7.13 (m, 12H), 6.88 (d, J 4.4 Hz, 1H), 4.49-4.36 (m, 5H), 4.22-4.01 (m, 9H), 4.01 (s, 3H), 3.74 (broad s, 2H), 3.46 (s, 3H), 3.26-3.21 (m, 1H), 3.06-2.92 (m, 13H), 2.82-2.68 (m, 2H), 2.58-2.45 (m, 6H), 2.18-1.75 (m, 15H), 1.42-1.29 (m, 2H), 1.13-1.04 (m, 7H), 0.74-0.56 (m, 9H). MS: 1584.4 [M+H]⁺.

### Example 9

### Synthesis of the Compound of Example 9

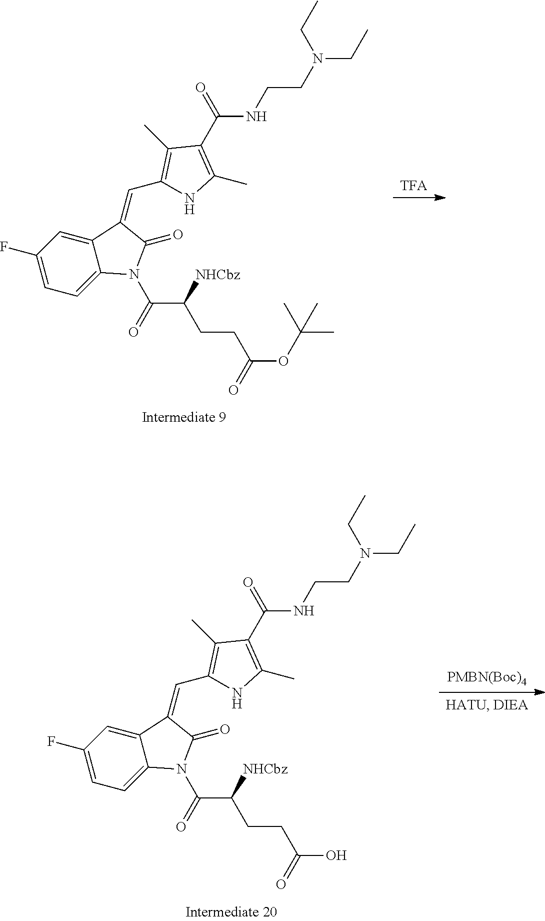

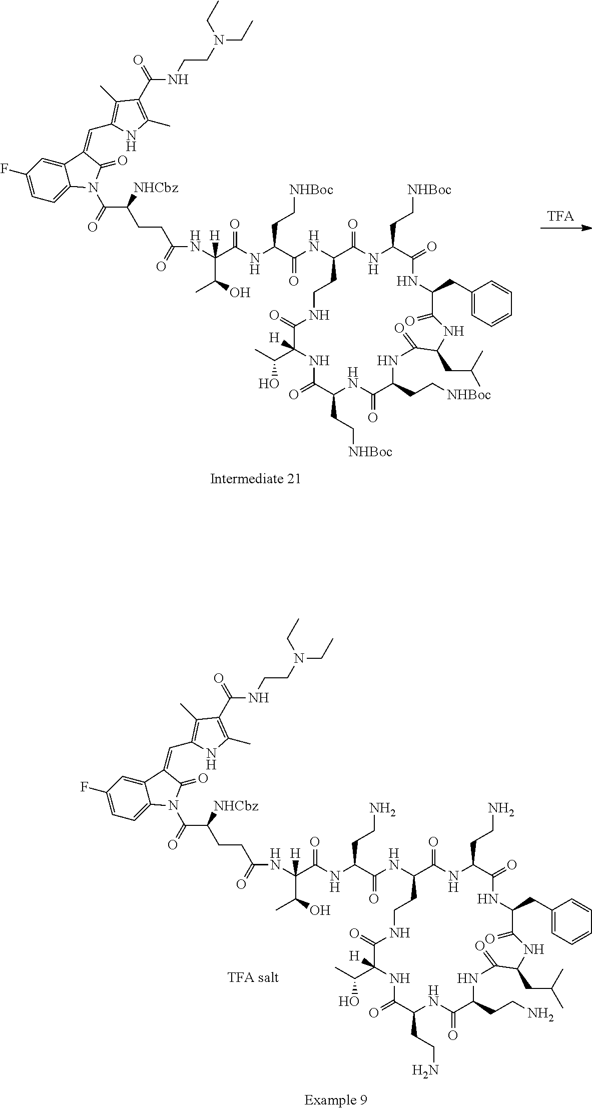

Intermediate 20. A mixture of Intermediate 9 (411 mg, 0.57 mmol) in 2 mL/10 mL of TFA/DCM was stirred at r.t. for 6 h, then evaporated and the crude product purified by HPLC to give 254 mg of the Intermediate 20.

Intermediate 21. A mixture of Intermediate 21 Intermediate 9 (221 mg, 0.33 mmol), PMBN(Boc)₄ (456 mg, 0.33 mmol), TEA (92 μL, 0.66 mmol), HATU (125 mg, 0.33 mmol) in 15 mL of THE was stirred at r.t. for 6 h. The reaction mixture was diluted with 200 mL of EA, and then washed with H₂O (20 mL×2), and brine (20 mL×2). The EA layer was dried and volatiles evaporated. The crude product was purified by HPLC to give 406 mg of the Intermediate 22.

Compound of Example 9. A mixture of the Intermediate 22 (123 mg, 0.06 mmol) in 0.5 mL/5 mL of TFA/DCM was stirred at r.t. for 4 h. Volatiles were evaporated, and the crude product was purified by HPLC to give 25 mg of the Compound of Example 9. NMR: 7.75-7.63 (m, 1H), 7.37-7.09 (m, 11H), 6.91-6.73 (m, 2H), 5.15-4.99 (m, 2H), 4.55-4.04 (m, 12H), 3.69-3.61 (m, 2H), 3.34-3.17 (m, 7H), 3.03-2.87 (m, 10H), 2.81-2.66 (m, 2H), 2.60-1.71 (m, 22H), 1.41-1.05 (m, 16H), 0.76-0.49 (m, 8H). MS: 1606.5 [M+H]⁺.

### Example 10

### Synthesis of the Compound of Example 10

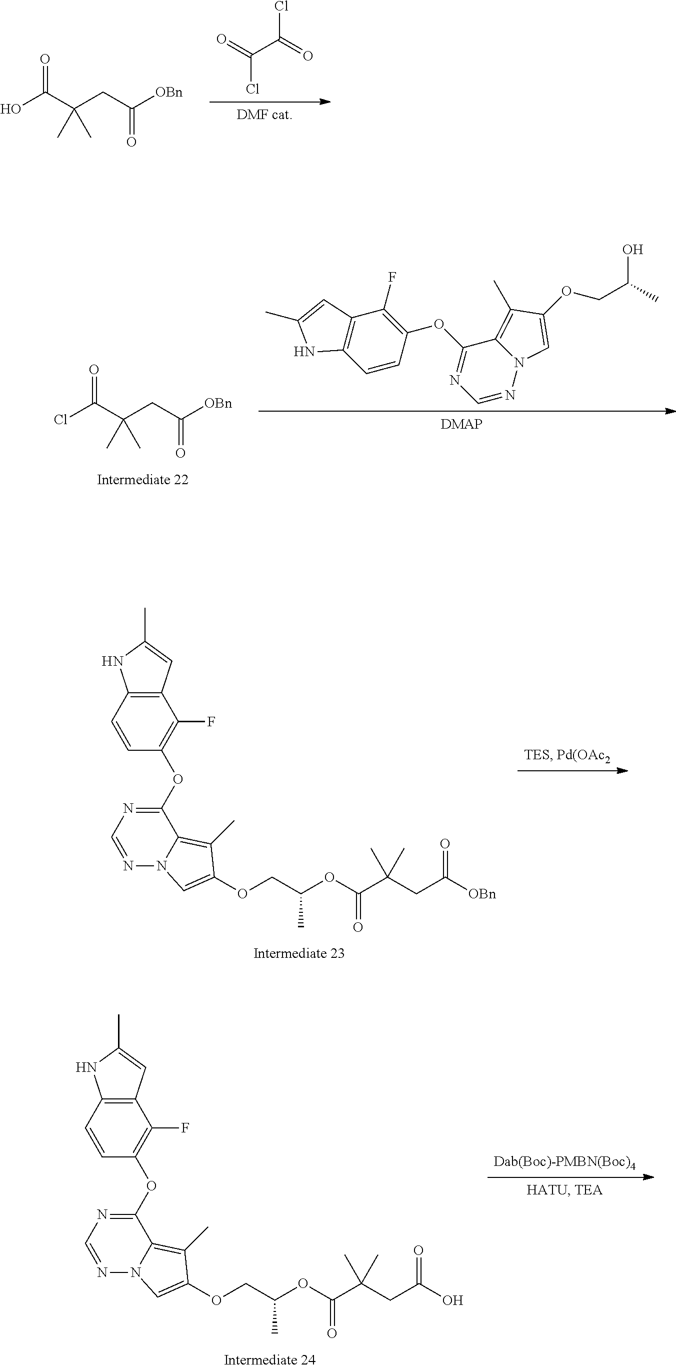

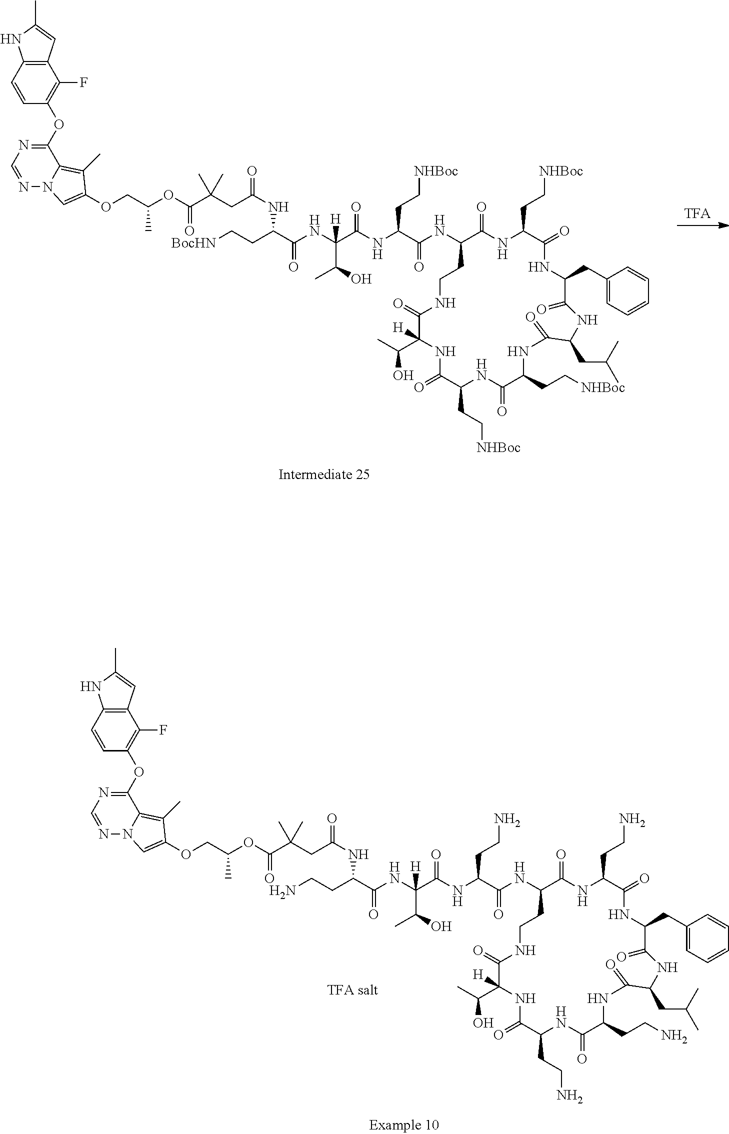

Intermediate 22. A mixture of 4-(benzyloxy)-2,2-dimethyl-4-oxobutanoic acid (1.2 g, 5.0 mmol), oxalyl chloride (0.95 mL, 7.5 mmol) and cat. DMF (3 drops) in 15 mL of DCM was stirred at r.t. for 1.5 h. Volatiles were evaporated to give the Intermediate 22, used directly at next step.

Intermediate 23. A solution of (R)-1-((4-((4-fluoro-2-methyl-1H-indol-5-yl)oxy)-5-methylpyrrolo[2,1-f][1,2,4]triazin-6-yl)oxy)propan-2-ol (206 mg, 0.56 mmol), the Intermediate 22 (145 mg, 0.56 mmol), and TEA (242 mg, 1.68 mmol) in 20 mL of DCM were stirred at r.t. for 16 h. Volatiles were evaporated, diluted with 150 mL of EA, and then washed with H₂O (20 mL×2), and brine (20 mL×2). The EA layer was dried and volatiles were evaporated. The crude product was purified by HPLC to give 158 mg of Intermediate 23.

Intermediate 24. A mixture of Intermediate 23 (113 mg, 0.2 mmol), Pd(OAc)₂ (5 mg, 0.02 mmol), TES (28 μL, 0.2 mmol), TES (116 mg, 1.0 mmol) in 10 mL of DCM was stirred at r.t. for 6 h. Volatiles were evaporated, diluted with 100 mL of EA, and then washed with H₂O (20 mL×2), and brine (10 mL×2). The EA layer was dried and volatiles evaporated. The crude product was purified by HPLC to give 69 mg of the Intermediate 24.

Intermediate 25. A mixture of Intermediate 24 (34 mg, 0.07 mmol), DAB(BOC)-PMBN(BOC)₄ (108 mg, 0.07 mmol), TEA (20 μL, 0.24 mmol), HATU (32 mg, 0.084 mmol) in 5 mL of DMF was stirred at r.t. for 16 h. The reaction mixture was diluted with 200 mL of EA, and then washed with H₂O (20 mL×2), and brine (20 mL×2). The EA layer was dried and volatiles evaporated. The crude product was purified by HPLC to give 56 mg of the Intermediate 25.

Compound of Example 10. A mixture of Intermediate 26 (100 mg, 0.05 mmol) in 0.5 mL/5 mL of TFA-DCM was stirred at r.t. for 3.5 h, then evaporated and the crude product purified by HPLC to give 26 mg of the Compound of Example 10. NMR: 7.60-6.85 (m, 10H), 4.57-4.34 (m, 6H), 4.24-3.94 (m, 14H), 3.27-2.68 (m, 22H), 2.40-1.74 (m, 23H), 1.41-1.07 (m, 22H), 0.76-0.55 (m, 9H). MS: 1543.7 [M+H]⁺.

### Example 11

### Synthesis of the Compound of Example 11

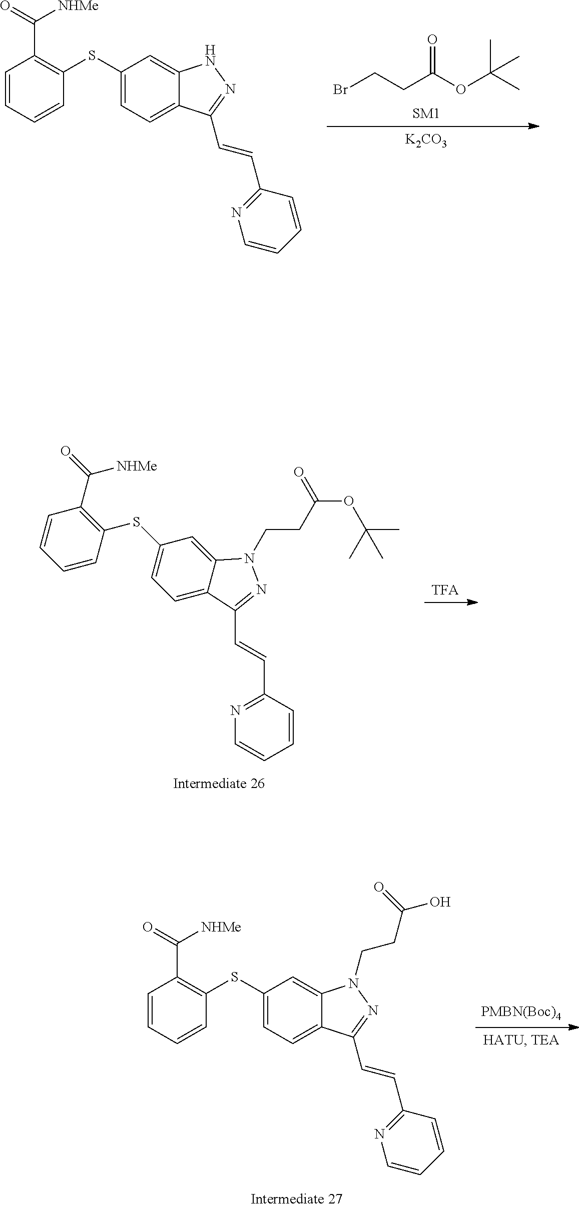

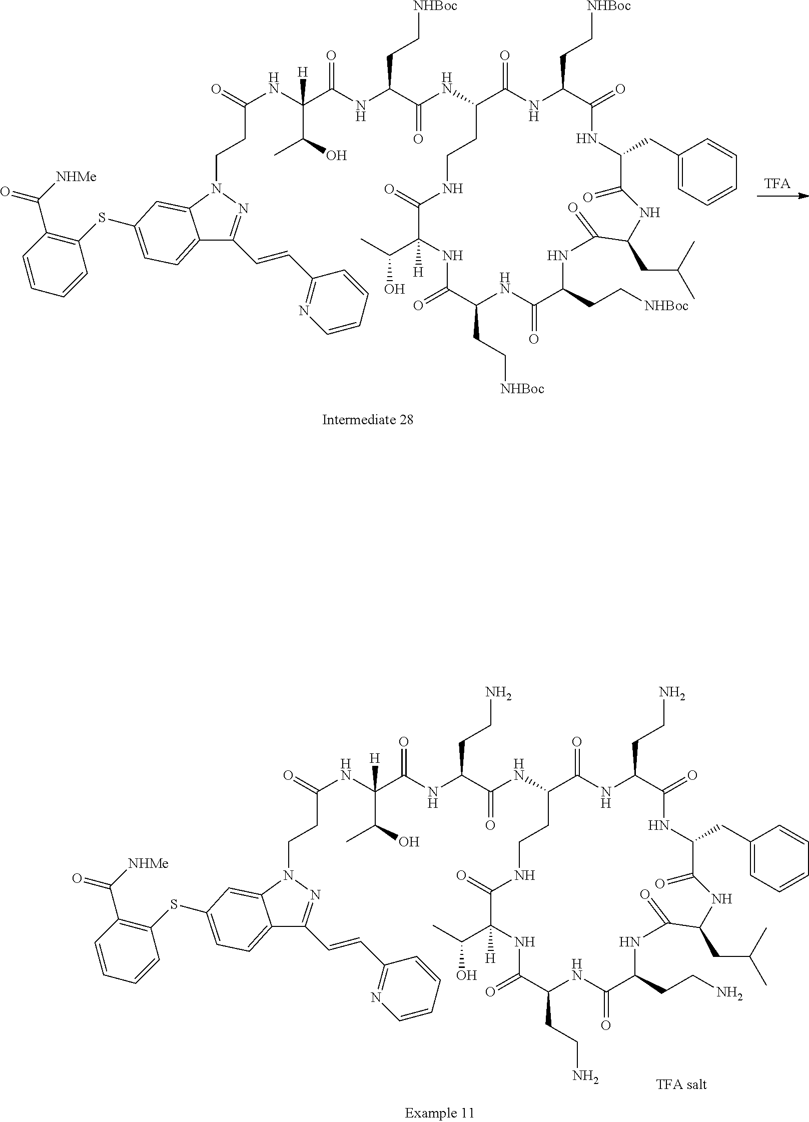

Intermediate 26. A solution of (E)-N-methyl-2-((3-(2-(pyridin-2-yl)vinyl)-1H-indazol-6-yl)thio)benzamide (386 mg, 1.0 mmol), K₂CO₃ (690 mg, 5.0 mmol) and SM1 (627 mg, 3.0 mmol) in 10 mL of DMF was stirred at 80° C. for 16 h. The reaction mixture was diluted with 250 mL of EA, and then washed with H₂O (30 mL×2), and brine (30 mL×2). The EA layer aws dried and volatiles evaporated. The crude product was purified by HPLC to give 288 mg of Intermediate 26.

Intermediate 27. A mixture of Intermediate 26 (288 mg, 0.56 mmol) in 2 mL/6 mL TFA/DCM was stirred at r.t. o.n. Volatiles were evaporated, the crude product was purified by HPLC to give 211 mg of Intermediate 27.

Intermediate 28 A mixture of Intermediate 27 (206 mg, 0.45 mmol), PMBN(Boc)₄ (409 mg, 0.3 mmol), DIEA (180 μL, 1.0 mmol), HATU (228 mg, 0.6 mmol) in 15 mL of DMF was stirred at r.t. for 6 h. The reaction mixture was diluted with 250 mL of EA, and then washed with H₂O (20 mL×2), and brine (20 mL×2). The EA layer was dried and volatiles evaporated. The crude product was purified by HPLC to give 408 mg of Intermediate 28.

Compound of Example 11. A mixture of crude Intermediate 28 (283 mg, 0.16 mmol) in 1 mL/10 mL of TFA/DCM was stirred at r.t. for 6 h. Volatiles were evaporated under vacuum, and the crude product was purified by HPLC to give 188 mg of the Compound of Example 11. NMR: 8.60-7.80 (m, 6H), 7.60-7.20 (m, 12H), 4.70-4.43 (m, 5H), 4.29-3.97 (m, 8H), 3.32-2.67 (m, 18H), 2.22-1.80 (m, 10H), 1.50-1.41 (m, 2H), 1.16 (d like, J 3.6 Hz, 3H), 0.83-0.61 (m, 10H). MS: 1403.6 [M+H]⁺.

### Example 12

### Synthesis of the Compound of Example 12

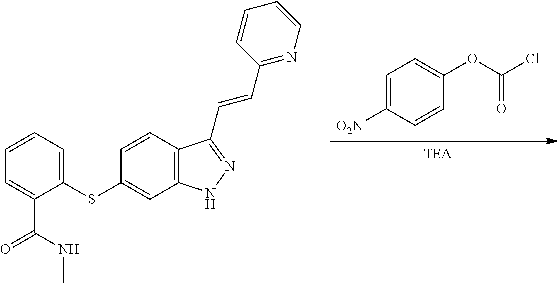

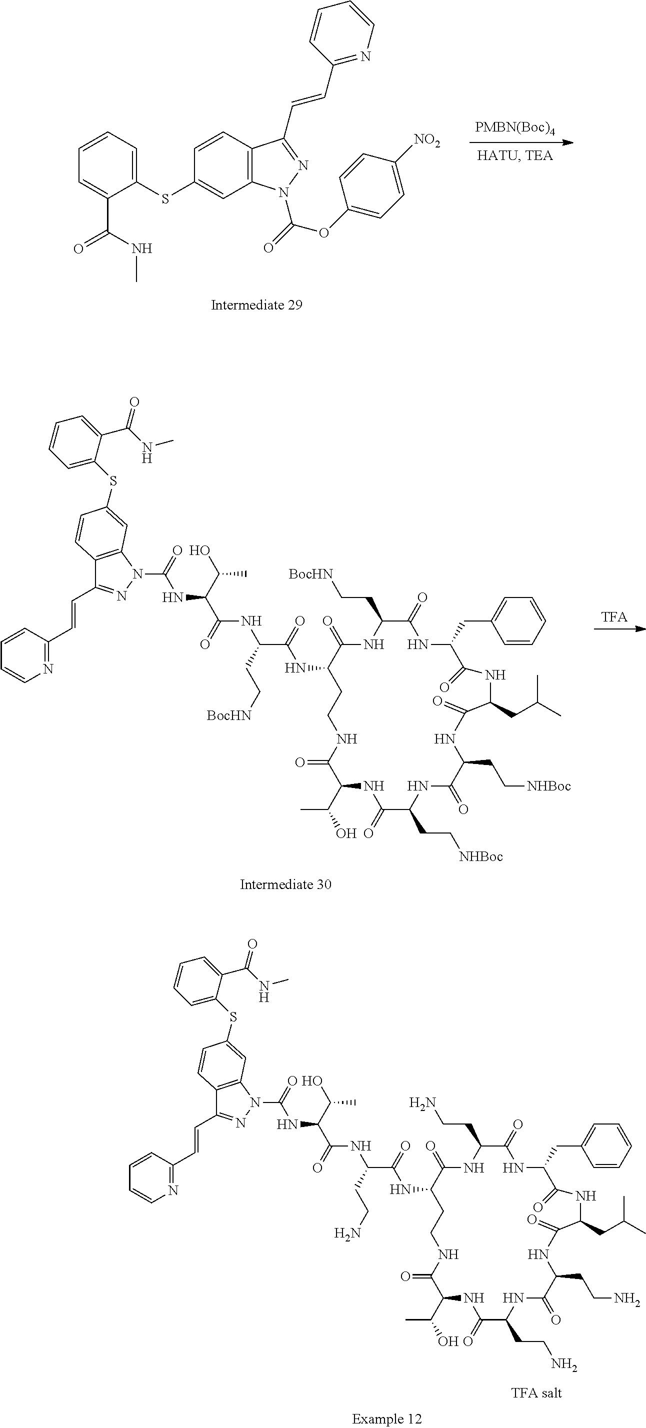

Intermediate 29. A 50 mL flask was charged with (E)-N-methyl-2-((3-(2-(pyridin-2-yl)vinyl)-1H-indazol-6-yl)thio)benzamide (386.5 mg, 1 mmol), PnpOC(═O)Cl (201.6 mg, 1 mmol), TEA (278 ul, 2 mmol) and dry DCM (10 mL) under Ar. The reaction mixture was stirred at r.t. overnight. Volatiles were removed under vacuum, then the residue was dried under vacuum to give crude Intermediate 29 as an off-white solid (680.2 mg).

Intermediate 30. A 25 mL flask was charged with Intermediate 28 (crude, 171.6 mg, MCP471-024, 0.252 mmol), PMBN(Boc)₄ (343.7 mg, 0.252 mmol), HATU (105.4 mg, 0.277 mmol), DIEA (129.4 ul, 0.756 mmol) and dry DMF (5 mL) under Ar. The reaction mixture was stirred at r.t. for 16 h. The reaction mixture was evaporated under vacuum, dried under vacuum, and then purified through a silica gel chromatography eluting with MeOH/DCM (0˜10% in 50 min, 15 mL/min) to give Intermediate 30 as an off-white solid (303.2 mg).

Compound of Example 12. A 25 mL flask was treated with Intermediate 30 (151.6 mg, 0.0854 mmol), TFA (1 mL) and DCM/DCE (2.5/2.5 mL) under Ar. The reaction mixture was stirred at r.t. for 5 h, LC-MS showed the reaction was completed. Volatiles were removed under vacuum, then dried under vacuum to give crude product, which was purified through C-18 chromatography eluting with ACN/Water (0.05% TFA, 0˜100% in 60 min, 18 mL/min) and lyophilized directly gave a white solid (67.5 mg). NMR: 8.60 (d, J=4.4 Hz, 1H), 8.46 (t, J=5.2 Hz, 1H), 8.25 (d, J=5.6 Hz, 1H), 7.97 (s, 1H), 7.91 (t, J=7.2 Hz, 2H), 7.83 (t, J=4.4 Hz, 1H), 7.71 (d, J=10.8 Hz, 1H), 7.47 (s, 1H), 7.43 (s, 3H), 7.31˜7.23 (m, 6H), 7.17 (t, J=7.6 Hz, 3H), 4.50˜4.37 (m, 8H), 4.23˜4.09 (m, 8H), 3.96 (t, J=3.6 Hz, 1H), 3.83 (d, J=4.0 Hz, 1H), 3.19˜3.14 (m, 1H), 3.08˜2.94 (m, 15H), 2.83˜2.68 (m, 5H), 2.60 (s, 3H), 2.24 (t, J=4.4 Hz, 1H), 2.18-2.13 (m, 7H), 2.07-1.94 (m, 5H), 1.87-1.71 (m, 5H), 1.52˜1.48 (m, 1H), 1.39 (t, J=6.8 Hz, 1H), 1.32 (d, J=7.2 Hz, 1H), 1.28 (d, J=4.4 Hz, 3H), 1.22˜1.17 (m, 3H), 1.12 (t, J=4.4 Hz, 2H), 0.94 (d, J=4.0 Hz, 3H), 0.74 (t, J=5.2 Hz, 3H), 0.67 (t, J=5.6 Hz, 5H), 0.60 (t, J=8.0 Hz, 5H). MS: 1375.5 [M+H]⁺.

### Example 13

### Synthesis of the Compound of Example 13

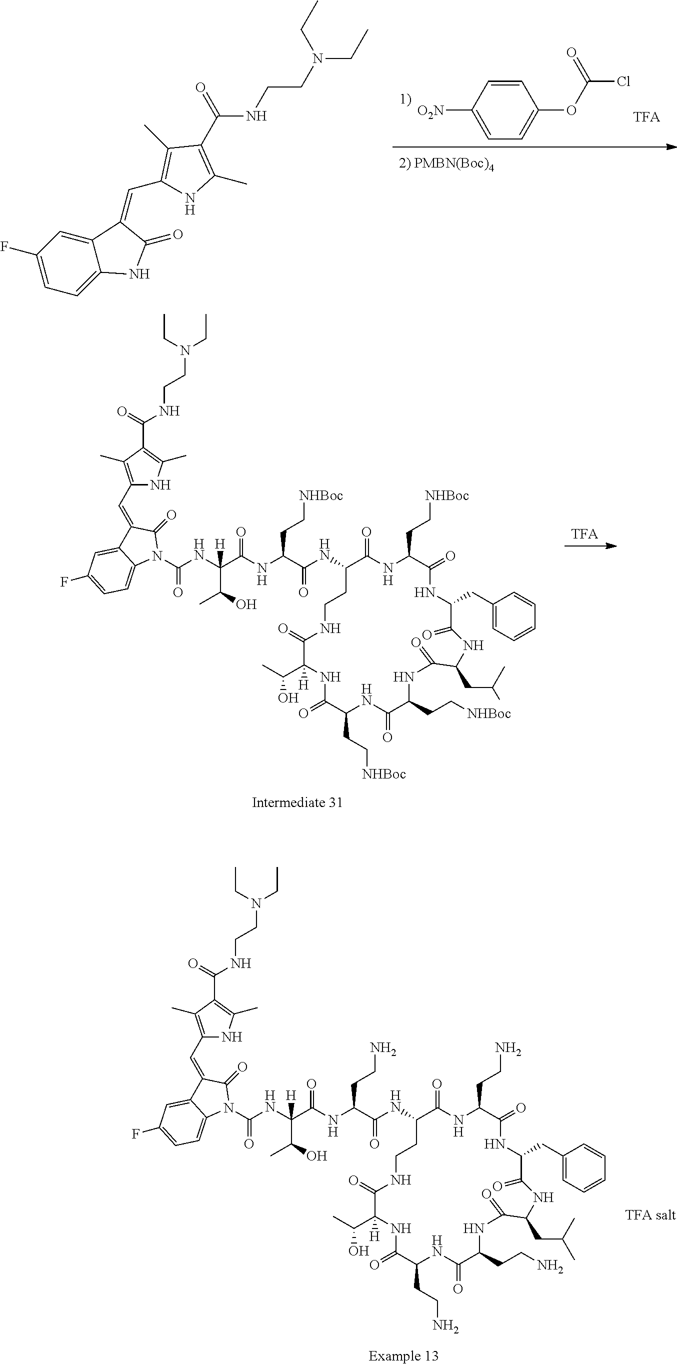

Intermediate 31. The mixture of (Z)—N-(2-(diethylamino)ethyl)-5-((5-fluoro-2-oxoindolin-3-ylidene)methyl)-2,4-dimethyl-1H-pyrrole-3-carboxamide (100 mg, 0.25 mmol), 4-nitrophenyl chloroformate (56 mg, 0.276 mmol) and DMAP (34 mg, 0.276 mmol) in DMF (3 mL) was stirred under Ar at r.t. for 3 h, then PMBN(Boc)₄ (341 mg, 0.25 mmol) was added to the reaction. Stirred at r.t. o.n. The mixture was extracted with EA (30 mL), washed with H₂O (5 mL×2) and brine (5 mL). The EA layer was dried and evaporated. The product was purified by HPLC (ACN/H₂O=0˜100%) to give 0.19 g of Intermediate 31. MS: 1788.77 (M+1).

Compound of Example 13. The mixture of Intermediate 31 (0.19 g, 0.107 mmol) in TFA/DCM (1.6/5.4 mL) was stirred at r.t for 1.5 h. The mixture was removed solvent and purified by C18 chromatography (ACN/H2O=0˜50%) to give 194 mg of the Compound of Example 13.

NMR: 7.72 (s, 1H), 7.44 (s, 1H), 7.31-7.22 (m, 5H), 7.15 (d, J=7.3 Hz, 2H), 6.86 (t, J=9.3 Hz, 1H), 4.53 (dd, J=9.3, 5.0 Hz, 1H), 4.45 (t, J=8.2 Hz, 1H), 4.41 (d, J=3.4 Hz, 1H), 4.38 (dd, J=6.4, 3.4 Hz, 1H), 4.34 (dd, J=8.8, 5.6 Hz, 1H), 4.11 (ddd, J=21.7, 10.1, 4.6 Hz, 3H), 4.04 (dd, J=10.3, 4.2 Hz, 1H), 3.77 (d, J=3.7 Hz, 1H), 3.69 (t, J=6.7 Hz, 2H), 3.32 (t, J=6.6 Hz, 2H), 3.25 (q, J=7.3 Hz, 4H), 3.11 (dd, J=13.7, 6.9 Hz, 1H), 2.97 (ddq, J=29.9, 14.5, 9.6, 7.6 Hz, 10H), 2.82-2.75 (m, 1H), 2.61 (d, J=9.6 Hz, 2H), 2.37 (s, 3H), 2.33 (s, 3H), 2.10 (td, J=16.7, 15.6, 9.7 Hz, 5H), 1.99 (ddd, J=20.3, 9.6, 5.1 Hz, 4H), 1.69 (s, 2H), 1.35 (d, J=6.2 Hz, 4H), 1.26 (t, J=7.3 Hz, 7H), 0.79 (d, J=6.5 Hz, 3H), 0.68 (d, J=7.0 Hz, 2H), 0.65 (d, J=6.2 Hz, 3H), 0.58 (d, J=6.1 Hz, 3H). MS: 1409.6 [M+Na]⁺.

### Example 14

### Synthesis of the Compound of Example 14

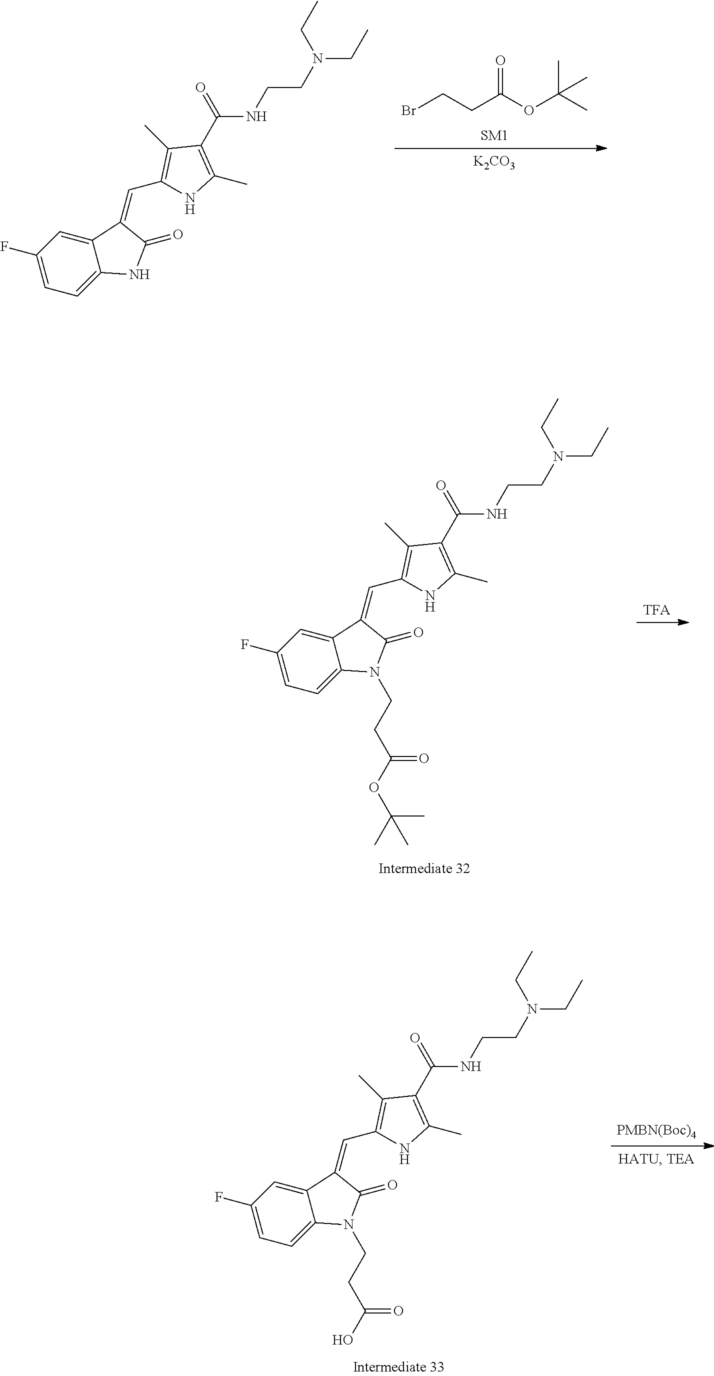

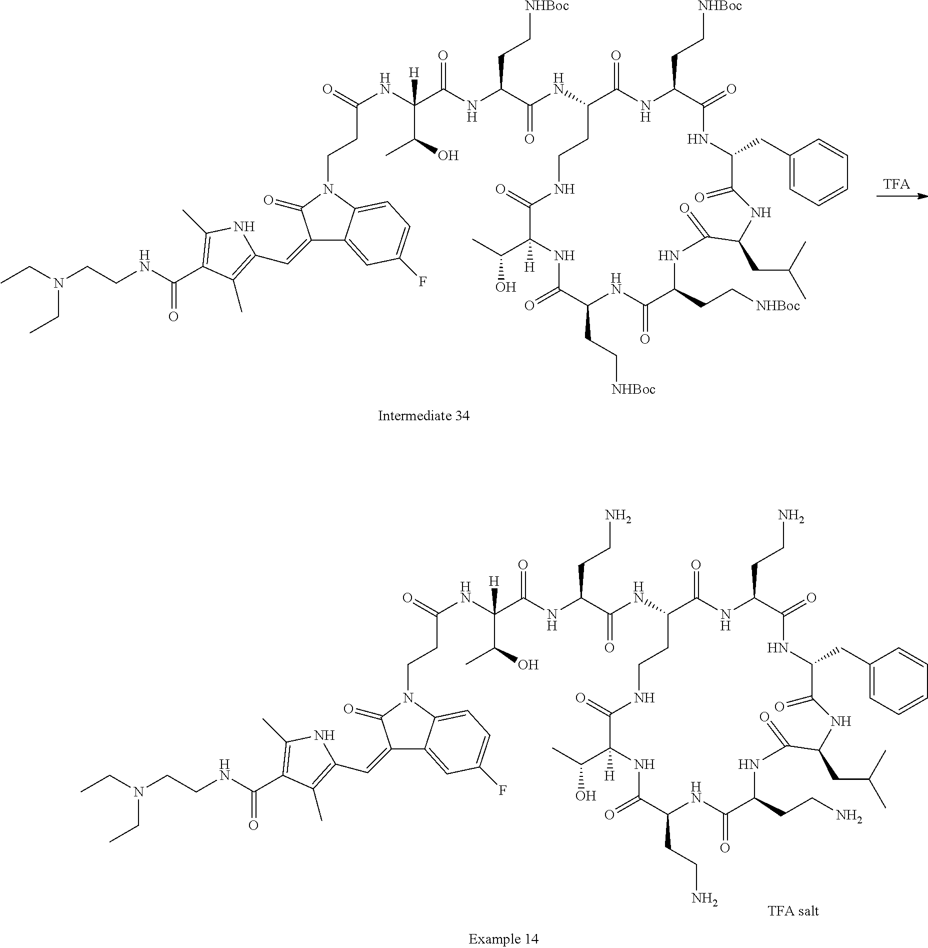

Intermediate 321. To a solution of (Z)—N-(2-(diethylamino)ethyl)-5-((5-fluoro-2-oxoindolin-3-ylidene)methyl)-2,4-dimethyl-1H-pyrrole-3-carboxamide (398 mg, 1.0 mmol), K₂CO₃ (690 mg, 5.0 mmol) and SM1 (627 mg, 3.0 mmol) in 10 mL of DMF was stirred at 80° C. for 16 h. The reaction mixture was diluted with 250 mL of EA, and then washed with H₂O (30 mL×2), brine (30 mL×2), dried and evaporated. The crude product was purified by HPLC to give 289 mg of Intermediate 32.

Intermediate 33. A mixture of Intermediate 32 (289 mg, 0.55 mmol) in 2 mL/6 mL of TFA/DCM was stirred at r.t. o.n. Volatiles were evaporated, the crude product was purified by HPLC to give 231 mg of Intermediate 33.

Intermediate 34. A mixture of Intermediate 33 (282 mg, 0.6 mmol), PMBN(Boc)₄ (545 mg, 0.4 mmol), DIEA (222 μL, 1.2 mmol), HATU (305 mg, 0.8 mmol) in 15 mL of DMF was stirred at r.t. for 6 h. The reaction mixture was diluted with 250 mL of EA, and then washed with H₂O (20 mL×2), brine (20 mL×2), dried and evaporated. The crude product was purified by HPLC to give 399 mg of Intermediate 34.

Compound of Example 14. A mixture of crude Intermediate 34 (383 mg, 0.2 mmol) in 2 mL/15 mL of TFA/DCM was stirred at r.t. for 6 h. Volatiles were evaporated, the crude product was purified by HPLC to afford the Compound of Example 14 (234 mg). NMR: 7.38-7.22 (m, 7H), 6.94-6.86 (m, 2H), 4.55 (t, J 5.2 Hz, 1H), 4.42-4.36 (m, 2H), 4.30-3.98 (m, 10H), 3.77 (t, J 4.0 Hz, 2H), 3.42-3.27 (m, 7H), 3.13-3.01 (m, 9H), 2.86-2.62 (m, 4H), 2.42 (s, 3H), 2.38 (s, 3H), 2.27-1.79 (m, 10H), 1.50-1.35 (m, 8H), 1.15 (t, J 4.0 Hz, 3H), 0.85-0.69 (m, 9H). MS: 1415.7 [M+H]⁺.

### Example 15

### Synthesis of the Compound of Example 15

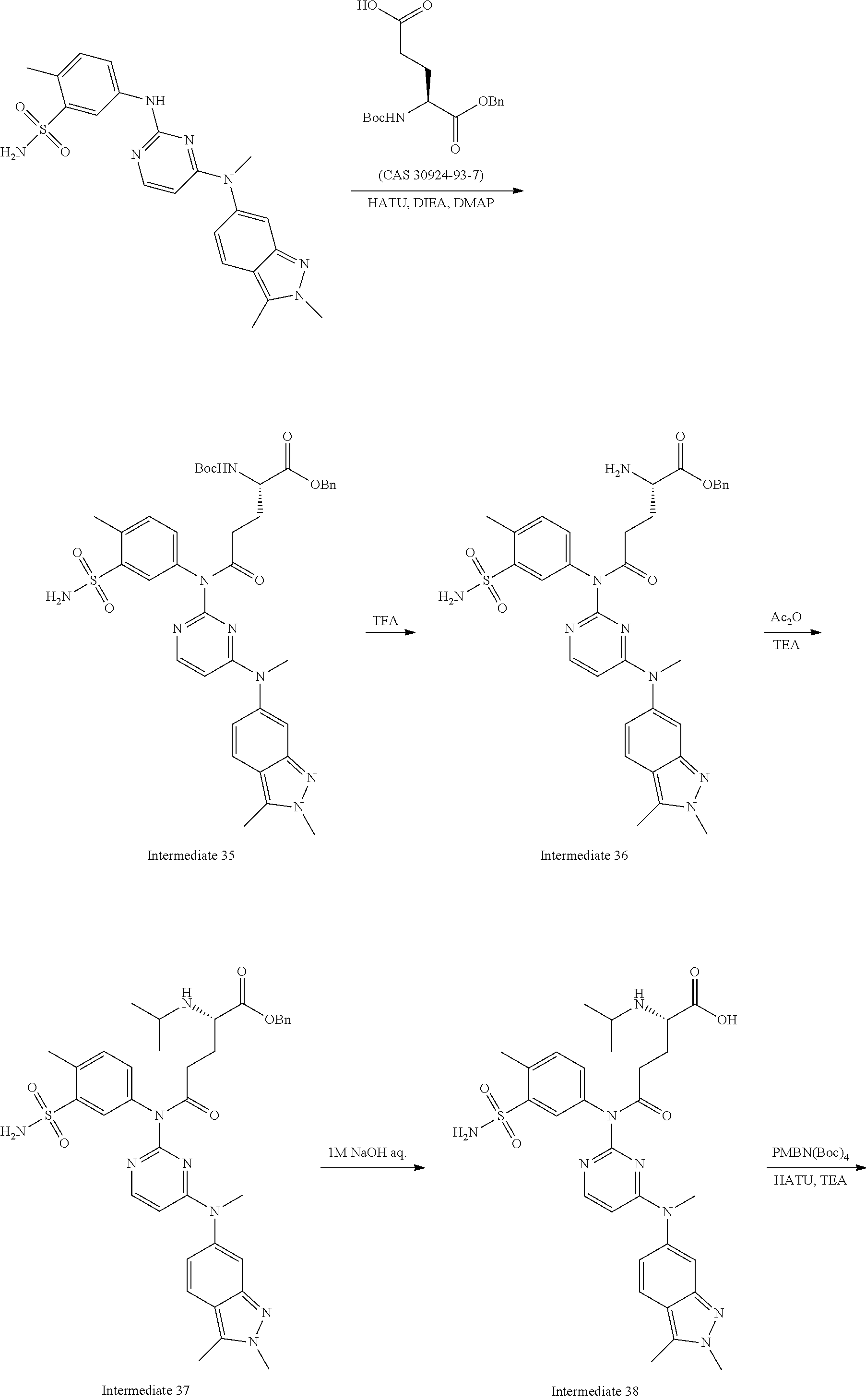

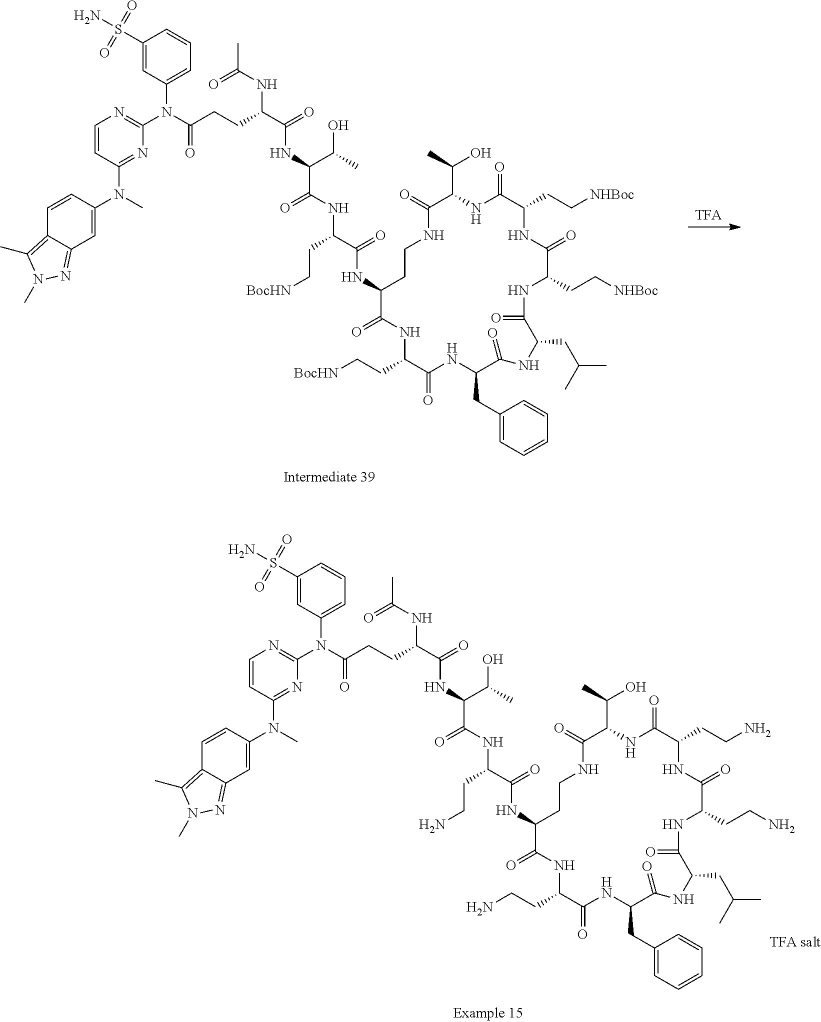

Intermediate 35. A 25 mL flask was charged with 5-((4-((2,3-dimethyl-2H-indazol-6-yl)(methyl)amino)pyrimidin-2-yl)amino)-2-methylbenzenesulfonamide (437.5 mg, 1 mmol), (S)-5-(benzyloxy)-4-((tert-butoxycarbonyl)amino)-5-oxopentanoic acid (371.1 mg, 1.1 mmol), HATU (570.4 mg, 1.5 mmol), DMAP (11.0 mg, cat.), DIEA (342 ul, 2 mmol) and dry DMF (5 mL) under Ar. The reaction mixture was stirred at r.t. for 16 h, LCMS showed the reaction completed. The reaction mixture was quenched with sat. aq. NH₄Cl solution and extracted with EA. The organic phase was washed with 5% NaCl aq. sol. twice, brine once, dried over anhydrous Na₂SO₄ and evaporated under vacuum to give crude product. Purification by HPLC eluting with ACN/Water (0˜100% in 60 min, 15 mL/min) gave Intermediate 35 as a white solid (168.3 mg).

Intermediate 36. A 25 mL flask was charged with Intermediate 35 (100.3 mg, 0.1325 mmol), TFA (0.2 mL) and DCM (1 mL) under N₂. Then the reaction mixture was stirred at r.t. for 3 h, LC-MS showed the reaction was completed. The reaction mixture was evaporated under vacuum, then dried under vacuum to give crude product as a white solid (164.1 mg).

Intermediate 37. A 25 mL flask was charged with Intermediate 36 (crude, 164 mg, 0.1325 mmol), Ac₂O (18.8 ul, 0.1988 mmol), TEA (55.2 ul, 0.3975 mmol) and DCM (2 mL) under Ar. Then the reaction mixture was stirred at r.t. for 4 h, LC-MS showed the reaction was complete. The reaction mixture was diluted with DCM (10 mL), washed with water, brine, dried over Na₂SO₄, filtered, evaporated under vacuum to give crude product. Purification by HPLC with ACN/Water (0-100% in 60 min, 10 mL/min) gave Intermediate 37 as a white solid (57.2 mg).

Intermediate 38. A 10 mL flask was charged with Intermediate 37 (59.8 mg, 0.0818 mmol), 1M NaOH aq. solution (409 ul, 0.4090 mmol, 5 eq.) and THE (2 mL). The reaction mixture was stirred at r.t. for 4 h, LC-MS showed the reaction was very slow. The reaction was added 1 M NaOH aq. solution (820 μL, 10 eq.) and continued stirring at r.t. for 6 h. LCMS showed the reaction was completed. The reaction mixture was quenched with 1 M HCl (1.23 mL, 15 eq.), THE was removed under vacuum, the residue was purified through C-18 chromatography with ACN/water (0˜100% in 60 min, 10 mL/min) to provide Intermediate 38 as a pale-yellow solid (28.5 mg).

Intermediate 39. A 10 mL flask was charged with Intermediate 38 (28.5 mg, 0.0468 mmol), PMBN(Boc)₄ (70.2 mg, 0.0515 mmol), HATU (19.6 mg, 0.0515 mmol), DIEA (16.1 μl, 0.0936 mmol) and dry DMF (1.5 mL) under Ar. The reaction mixture was stirred at r.t. for 16 h, LCMS showed the reaction was complete. The reaction mixture was quenched with 1 M HCl (1.1 eq.), diluted with EA, washed with brine, dried over anhydrous Na₂SO₄ and evaporated under vacuum. Purification by HPLC eluting with ACN/Water (0˜100% in 60 min, 10 mL/min) gave Intermediate 39 as a white solid (56.9 mg).

Compound of Example 15. A 10 mL flask was charged with Intermediate 39 (56.9 mg, 0.029 mmol), TFA (0.1 mL) and DCE (2 mL) under Ar. The reaction mixture was stirred at r.t. for about 2-3 h. Volatiles were removed under vacuum, residue dried under vacuum to give crude product, which was purified by HPLC eluting with ACN/Water (0.05% TFA, 0˜100% in 60 min, 10 mL/min) to afford the Compound of Example 15 as a white solid (22.0 mg). NMR: (400 MHz, D₂O, ppm): δ 7.74 (s, 2H), 7.48-7.40 (m, 6H), 7.26-7.20 (m, 7H), 7.11 (d, J=8.0 Hz, 4H), 6.86 (d, J=9.2 Hz, 2H), 4.47 (dd, J=13.2, 7.6 Hz, 2H), 4.40˜4.34 (m, 3H), 4.31˜4.27 (m, 1H), 4.20˜4.16 (m, 5H), 4.14˜4.08 (m, 12H), 4.03 (t, J=7.6 Hz, 2H), 3.98 (s, 6H), 3.45 (s, 6H), 3.26-3.18 (m, 2H), 3.06-2.92 (m, 20H), 2.81˜2.73 (m, 2H), 2.72˜2.64 (m, 2H), 2.55 (s, 6H), 2.47-2.39 (m, 9H), 2.18-2.07 (m, 9H), 2.04-1.92 (m, 7H), 1.82 (d, J=8.8 Hz, 9H), 1.40-1.27 (m, 4H), 1.05 (dd, J=9.2, 2.8 Hz, 12H), 0.64 (t, J=3.2 Hz, 6H), 0.57 (t, J=3.2 Hz, 6H). MS: 1553.6 [M+H]⁺.

### Example 16

### Synthesis of the Compound of Example 16

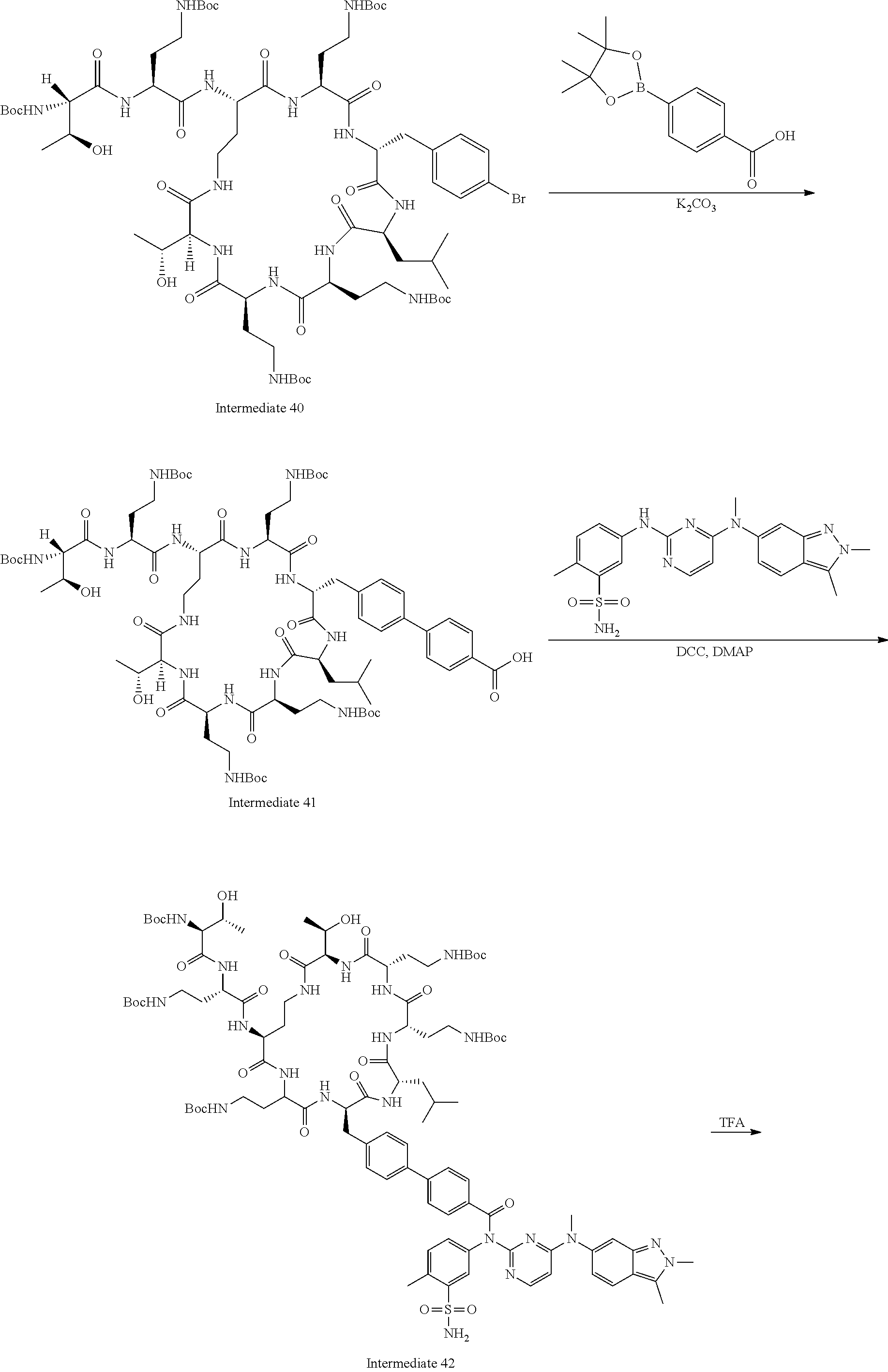

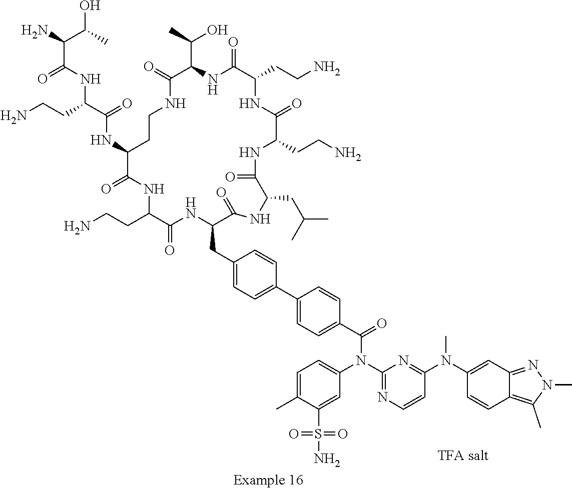 [SMILES: Cc1ccc(N(C(=O)c2ccc(-c3ccc(C[C@H]4NC(=O)C(CCN)NC(=O)[C@@H](NC(=O)[C@H](CCN)NC(=O)[C@@H](N)[C@@H](C)O)CCNC(=O)[C@@H]([C@@H](C)O)NC(=O)[C@H](CCN)NC(=O)[C@H](CCN)NC(=O)[C@H](CC(C)C)NC4=O)cc3)cc2)c2nccc(N(C)c3ccc4c(C)n(C)nc4c3)n2)cc1S(N)(=O)=O]

Intermediate 41. A solution of Intermediate 40 (127 mg, 0.08 mmol), K₂CO₃ (22 mg, 0.16 mmol), Pd(dppf)Cl₂ (2 mg, 0.008 mmol), and 4-(4,4,5,5-tetramethyl-1,3,2-dioxaborolan-2-yl)benzoic acid (19 mg, 0.08 mmol) in 2 mL of DMF was stirred at 85° C. for 16 h. The reaction mixture was diluted with 50 mL of EA, and then washed with H₂O (10 mL×2), and brine (10 mL×2). The EA layer was dried and evaporated. The crude product was purified by HPLC to give 115 mg of Intermediate 41.

Intermediate 42. A mixture of Intermediate 41 (115.3 mg, 0.1 mmol), 5-((4-((2,3-dimethyl-2H-indazol-6-yl)(methyl)amino)pyrimidin-2-yl)amino)-2-methylbenzenesulfonamide (87.6 mg, 0.2 mmol), DCC (618.3 mg, 0.3 mmol), DMAP (12 mg, 0.1 mmol) in 2 mL of DMF was stirred at 60° C. for 16 h. Volatiles were evaporated, diluted with 100 mL of EA, and then washed with H₂O (20 mL×2), and brine (10 mL×2). THe EA layer was dried and evaporated. The crude product was purified by HPLC to give 118 mg of crude Intermediate 42.

Compound of Example 16. A mixture of crude Intermediate 42 (118 mg) in 0.1 mL/2 mL of TFA/DCM was stirred at r.t. for 3 h. Volatiles were evaporated, and purified by HPLC to afford the Compound of Example 16 (11 mg). NMR: 7.90-7.83 (m, 2H), 7.67-7.47 (m, 7H), 7.29-7.16 (m, 7H), 6.75 (d, J 6.4 Hz, 1H), 4.53-4.40 (m, 4H), 4.20-4.10 (m, 8H), 3.96-3.86 (m, 5H), 3.30-3.25 (m, 1H), 3.15-2.87 (m, 17H), 2.58-2.37 (m, 5H), 2.14-1.77 (m, 13H), 1.22-1.07 (m, 10H), 0.37-0.19 (m, 9H). MS: 1502.8 [M+H]⁺.

### Example 17

### Synthesis of the Compound of Example 17

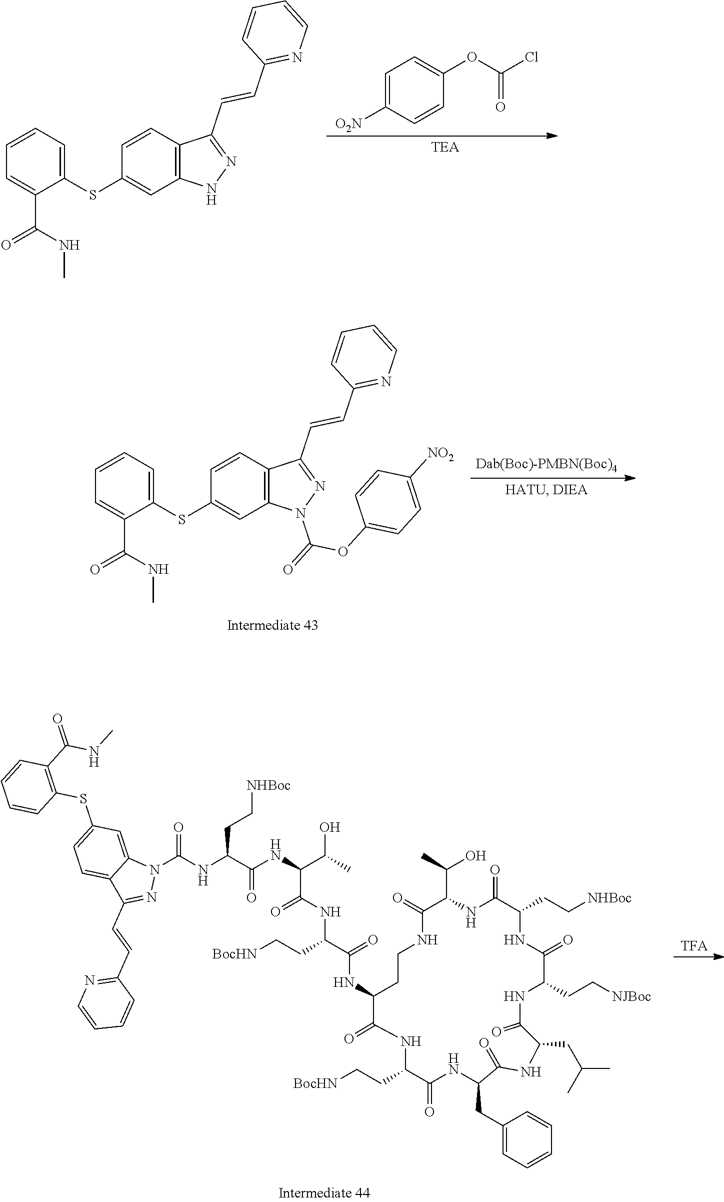

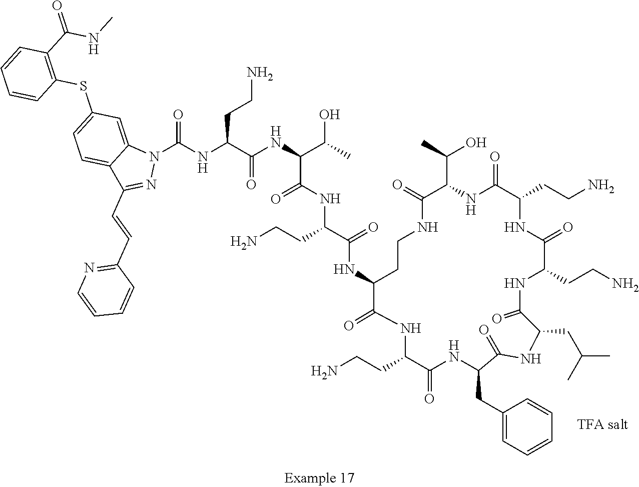 [SMILES: CNC(=O)c1ccccc1Sc1ccc2c(/C=C/c3ccccn3)nn(C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 43. A 50 mL flask was charged with (E)-N-methyl-2-((3-(2-(pyridin-2-yl)vinyl)-1H-indazol-6-yl)thio)benzamide (386.5 mg, 1 mmol), PnpOC(═O)Cl (p-nitrophenyl chkoroformate; 201.6 mg, 1 mmol), TEA (278 ul, 2 mmol) and dry DCM (10 mL) under Ar. Then the reaction mixture was stirred at r.t. overnight. Volatiles were removed under vacuum, then the residue dried under vacuum to give crude Intermediate 43 as an off-white solid (680.2 mg).

Intermediate 44. A 25 mL flask was charged with Intermediate 43 (crude, 196.4 mg, 0.289 mmol), Dab(Boc)-Thr-Dab(Boc)-cyclo[Dab(Boc)-Dab(Boc)-D-Phe-Leu-Dab(Boc)-Dab(Boc)-Thr] (same as Dab(Boc)PMBN(Boc)₄ (451.5 mg, 0.289 mmol), HATU (120.8 mg, 0.317 mmol), DIEA (148.3 ul, 0.866 mmol) and dry DMF (6 mL) under Ar. The reaction mixture was stirred at r.t. for 16 h. The reaction mixture was evaporated under vacuum, dried under vacuum, then purified through a silica chromatography eluting with MeOH/DCM (0˜10% in 50 min, 15 mL/min) to give Intermediate 44 as an off-white solid (375.2 mg).

Compound of Example 17. A 25 mL flask was charged with Intermediate 44 (187.6 mg, 0.0949 mmol), TFA (1 mL) and DCM/DCE (2.5/2.5 mL) under Ar. The reaction mixture was stirred at r.t. for 5 h. Volatiles were removed under vacuum, then dried under vacuum to give crude product. Purification by HPLC eluting with ACN/Water (0.05% TFA, 0˜100% in 60 min, 18 mL/min) and lyophilizing directly gave the compound of Example 17 as a white solid (110.2 mg). NMR: 8.59 (d, J=3.6 Hz, 1H), 8.45 (t, J=5.2 Hz, 1H), 8.24 (d, J=5.6 Hz, 1H), 7.95 (s, 1H), 7.90-7.82 (m, 3H), 7.64 (d, J=11.2 Hz, 1H), 7.48 (d, J=4.4 Hz, 1H), 7.41 (s, 3H), 7.28 (t, J=4.8 Hz, 2H), 7.23 (t, J=4.8 Hz, 1H), 7.20 (d, J=5.6 Hz, 1H), 7.15 (d, J=4.8 Hz, 2H), 4.64 (dd, J=6.4, 2.8 Hz, 2H), 4.46 (t, J=5.6 Hz, 1H), 4.42˜4.38 (m, 1H), 4.37 (d, J=2.8 Hz, 2H), 4.21˜4.08 (m, 8H), 3.28-3.23 (m, 1H), 3.14 (t, J=5.6 Hz, 2H), 3.08-2.96 (m, 10H), 2.95-2.93 (m, 2H), 2.83˜2.79 (m, 1H), 2.74-2.70 (m, 1H), 2.58 (s, 3H), 2.35˜2.29 (m, 1H), 2.26˜2.21 (m, 1H), 2.18˜2.12 (m, 5H), 2.10˜2.04 (m, 3H), 2.00˜1.93 (m, 1H), 1.88˜1.80 (m, 3H), 1.77˜1.74 (m, 2H), 1.41˜1.37 (m, 1H), 1.33˜1.27 (m, 1H), 1.13 (d, J=4.4 Hz, 3H), 1.08 (d, J=4.0 Hz, 3H), 0.66 (s, 5H), 0.58 (d, J=3.6 Hz, 4H). MS: 1498.3 [M+Na]+.

### Example 18

### Synthesis of the Compound of Example 18

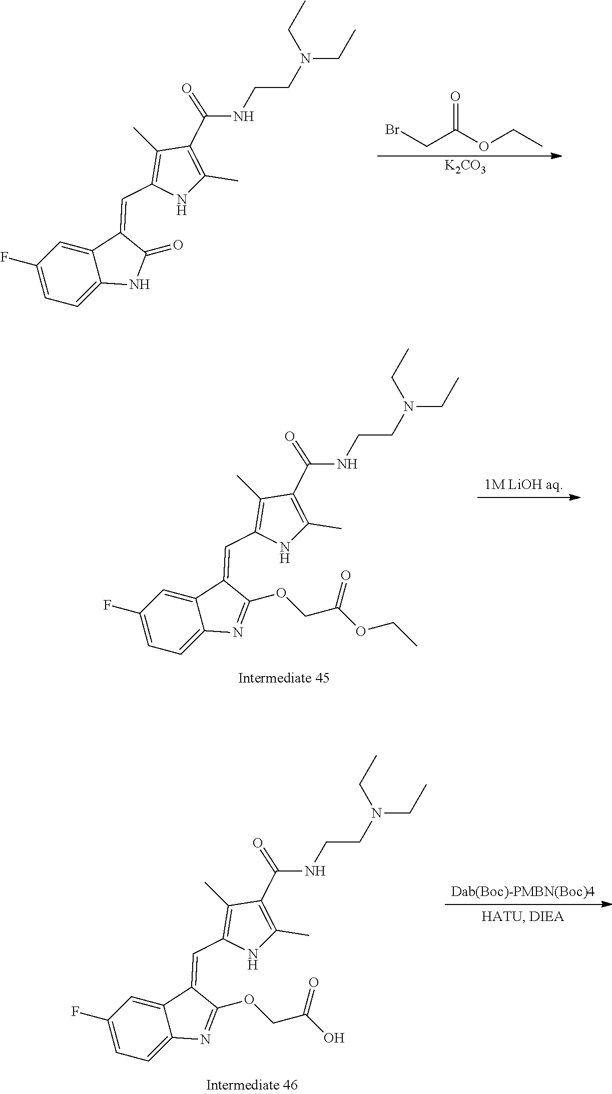

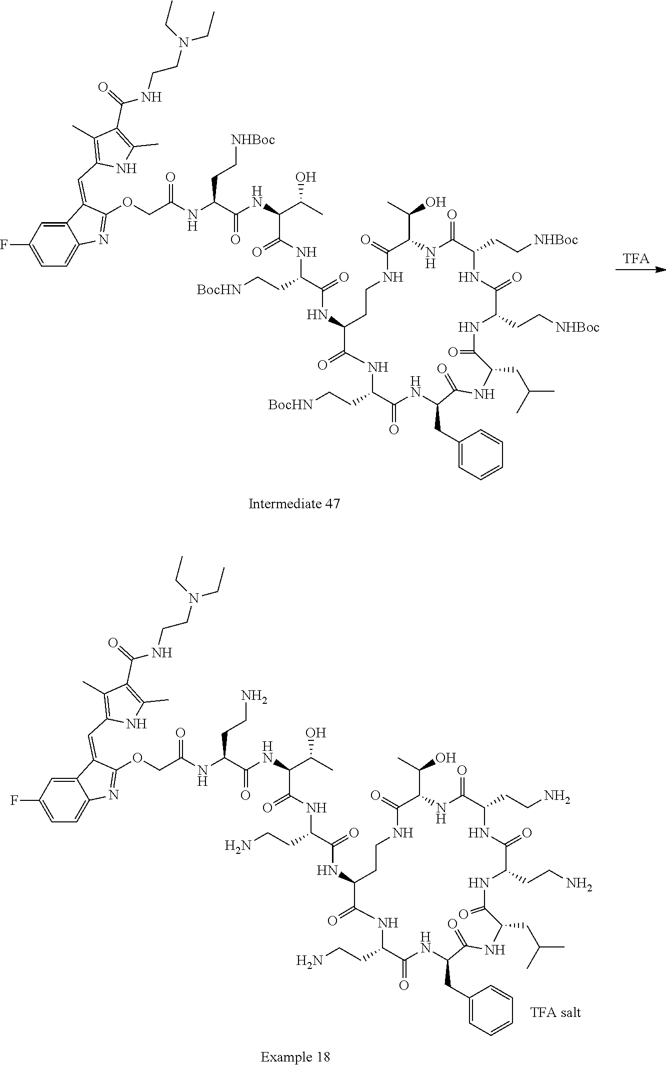

Intermediate 45. A 25 mL flask was charged with (Z)—N-(2-(diethylamino)ethyl)-5-((5-fluoro-2-oxoindolin-3-ylidene)methyl)-2,4-dimethyl-1H-pyrrole-3-carboxamide (266.3 mg, 0.5 mmol), ethyl 2-bromoacetate (55.4 ul, 0.5 mmol), K₂CO₃ (138.2 mg, 1 mmol) and dry DMF (5.2 mL) under Ar. Then the reaction mixture was stirred at r.t. overnight. The reaction mixture was quenched with water, extracted with EA for 3 times. The combined organic layers were washed with 5% NaCl aq. solution, brine, then dried over anhydrous Na₂SO₄, filtered and evaporated under vacuum to give crude product. Purification through a silica chromatography with MeOH/DCM (0˜10% in 50 min, 16 mL/min) gave Intermediate 45 as a yellow solid (69.0 mg).

Intermediate 46. A 25 mL flask was charged with Intermediate 45 (69.0 mg, 0.142 mmol), 1 M LiOH aq. solution (0.75 mL) and THE (1.5 mL) under Ar. The reaction mixture was stirred at r.t. for 16 h. The reaction was cooled to 0-5° C., and 1M HCl was added to adjust pH=5-6. Volatiles were removed under vacuum, the residue was dissolved in ACN/H₂O and purified by HPLC eluting with ACN/Water (0.05% TFA, 0˜100% in 50 min, 15 mL/min) then lyophilized directly to give Intermediate 46 as a pale-yellow solid (63.5 mg).

Intermediate 47. A 25 mL flask was charged with Intermediate 46 (63.5 mg, 0.139 mmol), Dab(Boc)-PMBN(Boc)₄ (239.3 mg, 0.153 mmol), HATU (58.2 mg, 0.153 mmol), DIEA (71.4 ul, 0.417 mmol) and dry DMF (1.5 mL) under Ar. The reaction mixture was stirred at r.t. for 16 h. The reaction mixture was diluted with water, extracted three times with EA. The combined EA layers were washed with 5% NaCl twice, and brine once and were dried over anhydrous Na₂SO₄. The filtrate was evaporated under vacuum, dried under vacuum, and then purified by HPLC eluting with ACN/Water (0.05% TFA, 0˜100% in 50 min, 15 mL/min) to give Intermediate 47 as a yellow solid (143.3 mg).

Compound of Example 18. A 25 mL flask was charged with Intermediate 47 (143.3 mg), TFA (0.4 mL) and DCE (2 mL) under Ar. The reaction mixture was stirred at r.t. for 6 h. Volatiles were removed under vacuum, then dried under vacuum to give crude product. Purification by HPLC eluting with ACN/Water (0.05% TFA, 0˜100% in 60 min, 15 mL/min) afforded the Compound of Example 18 as a yellow solid (126.2 mg). NMR: 7.36 (s, 1H), 7.28-7.21 (m, 6H), 7.13 (d, J=5.2 Hz, 3H), 6.92˜6.78 (m, 1H), 6.69 (d, J=10.4 Hz, 1H), 4.59 (t, J=12.0 Hz, 2H), 4.52˜4.43 (m, 3H), 4.37˜4.33 (m, 2H), 4.23˜4.22 (m, 1H), 4.19-4.16 (m, 3H), 4.11˜4.05 (m, 6H), 3.71 (t, J=4.4 Hz, 1H), 3.65 (t, J=4.4 Hz, 1H), 3.34 (t, J=4.8 Hz, 1H), 3.30 (t, J=4.4 Hz, 1H), 3.25-3.16 (m, 7H), 3.03˜2.94 (m, 16H), 2.76-2.64 (m, 3H), 2.26 (t, J=7.2 Hz, 4H), 2.16-2.01 (m, 10H), 1.93˜1.72 (m, 7H), 1.39˜1.36 (m, 1H), 1.31˜1.26 (m, 9H), 1.09 (s, 1H), 1.06 (d, J=5.6 Hz, 3H), 1.01 (d, J=4.0 Hz, 3H), 0.72 (s, 1H), 0.64 (d, J=3.6 Hz, 4H), 0.58 (d, J=4.0 Hz, 4H). MS: 1523.8 [M+Na]⁺.

### Example 19

### Synthesis of the Compound of Example 19

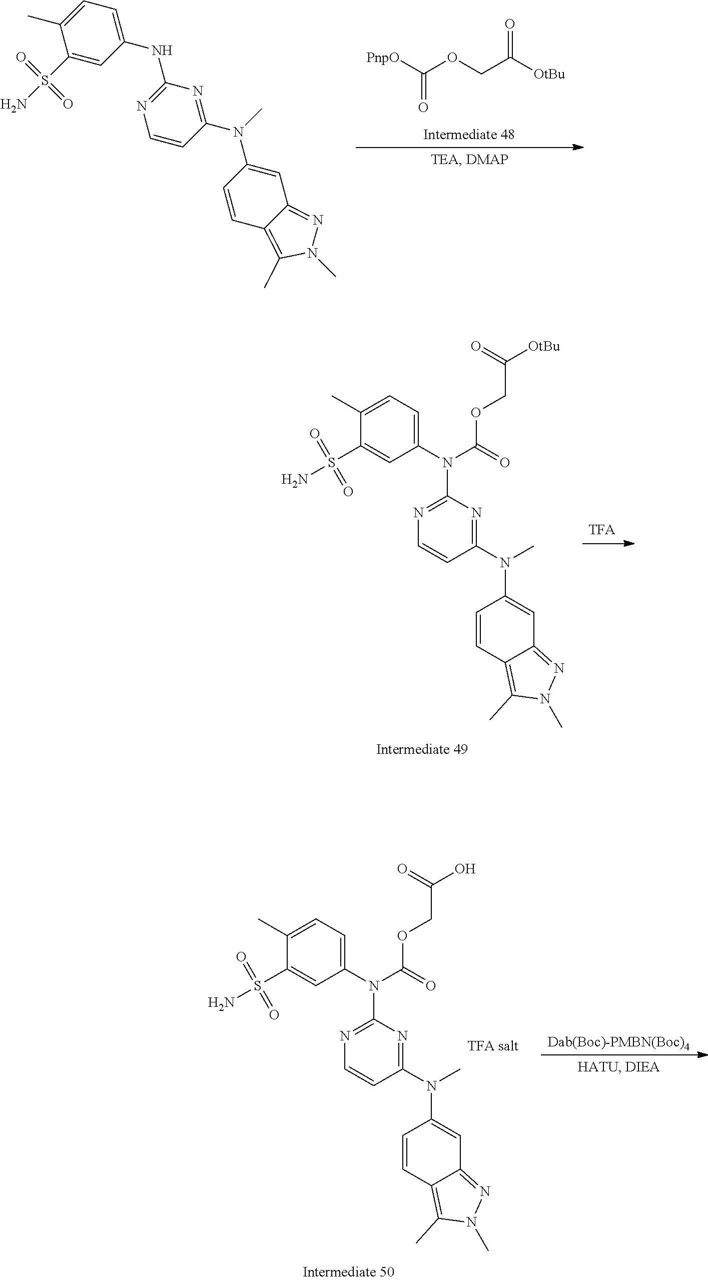

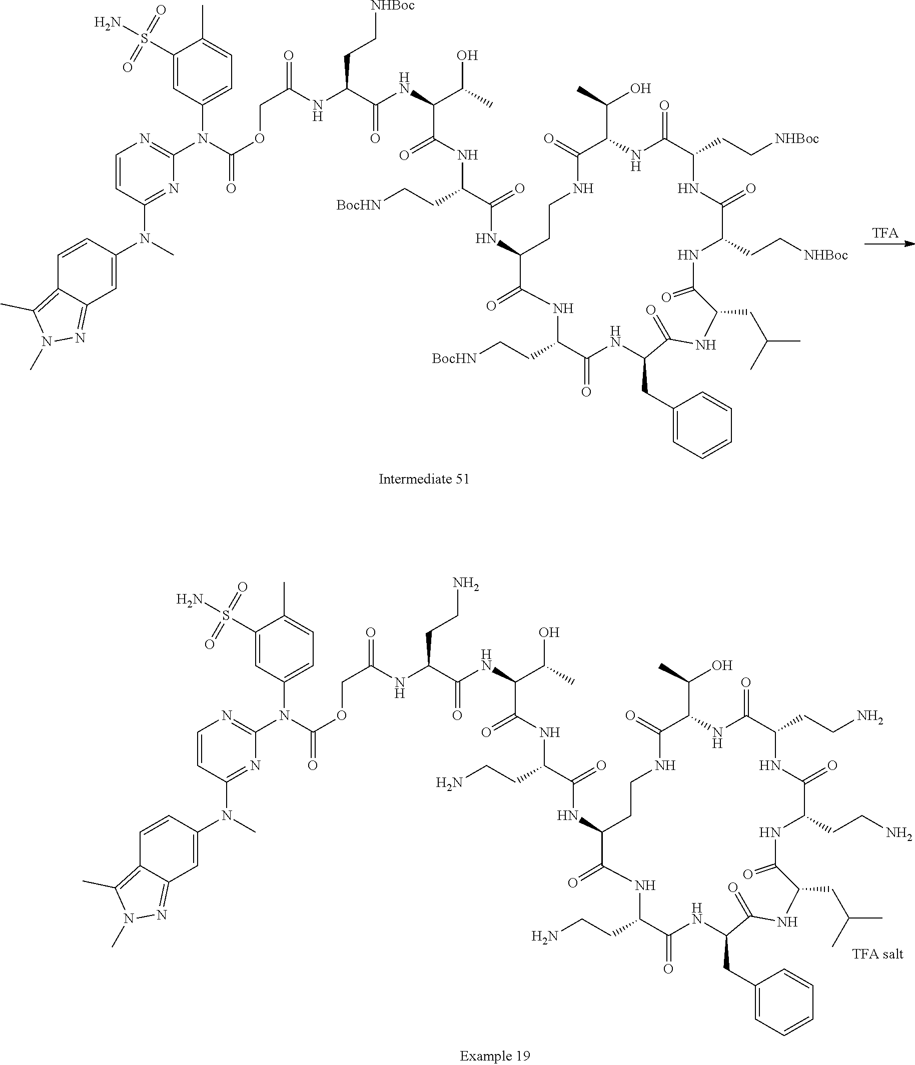

Intermediate 48. A 50 mL flask was charged with tert-butyl 2-hydroxyacetate (1.32 g, 10 mmol), PnpOC(═O)C₁ (2.02 g, 10 mmol) and dry DCM (13.2 mL) under Ar. Cooled to 0˜5° C., the reaction mixture was added TEA (2.78 mL, 20 mmol) slowly and stirred at r.t. for 2 h. Volatiles were removed under vacuum, and the residue was dried under vacuum for 1-2 h to give crude product as a yellow solid (Ca. 4.2 g).

Intermediate 49. A 10 mL flask was charged with 5-((4-((2,3-dimethyl-2H-indazol-6-yl)(methyl)amino)pyrimidin-2-yl)amino)-2-methylbenzenesulfonamide (87.4 mg, 0.2 mmol), Intermediate 48 (crude, 89.2 mg, 0.2 mmol), TEA (55.6 μl, 0.4 mmol), DMAP (2.4 mg, cat.) and dry DMF (1.5 mL) under Ar. The reaction mixture was stirred at r.t. for 16 h. The reaction mixture was diluted with EA, washed with 5% NaCl aq. sol. twice, and brine once, and then dried over anhydrous Na₂SO₄ and evaporated to give crude product. Purification by HPLC eluting with ACN/Water (0˜100% in 60 min, 15 mL/min) gave a white solid (78.1 mg).

Intermediate 50. A 25 mL flask was charged with Intermediate 49 (78.1 mg, 0.13 mmol, 1.0 eq.), TFA (1 mL) and DCE (1.0 mL) under Ar. The reaction mixture was stirred at r.t. for 3 h, LC-MS showed the reaction was OK. Volatiles were removed under vacuum and dried under vacuum to give a white solid (86.0 mg).

Intermediate 51. A 25 mL flask was charged with Intermediate 50 (crude, TFA salt, 86.0 mg, 0.1311 mmol), Dab(Boc)PMBN(Boc)₄ (225.4 mg, 0.14 mmol), HATU (54.83 mg, 0.14 mmol), DIEA (90 ul, 0.5244 mmol) and dry DMF (1.5 mL) under Ar. The reaction mixture was stirred at r.t. for 16 h. The reaction was worked up with EA/Water/brine and then purified by HPLC eluting with ACN/Water (0˜100% in 60 min, 15 mL/min) to give a white solid (115.4 mg).

Compound of Example 19. A 25 mL flask was charged with Intermediate 51 (110.4 mg, 1.0 eq.), TFA (1.0 mL) and DCE (5 mL) under Ar. The reaction mixture was stirred at r.t. for 2˜3 h. Volatiles were removed under vacuum, then dried under vacuum to give crude product. Purification by HPLC eluting with ACN/Water (0˜100% in 60 min, 15 mL/min) gave the Compound of Example 19 as a white solid (57.5 mg). MS: 1585.7 [M+H]⁺.

### Example 20

### Synthesis of the Compound of Example 20

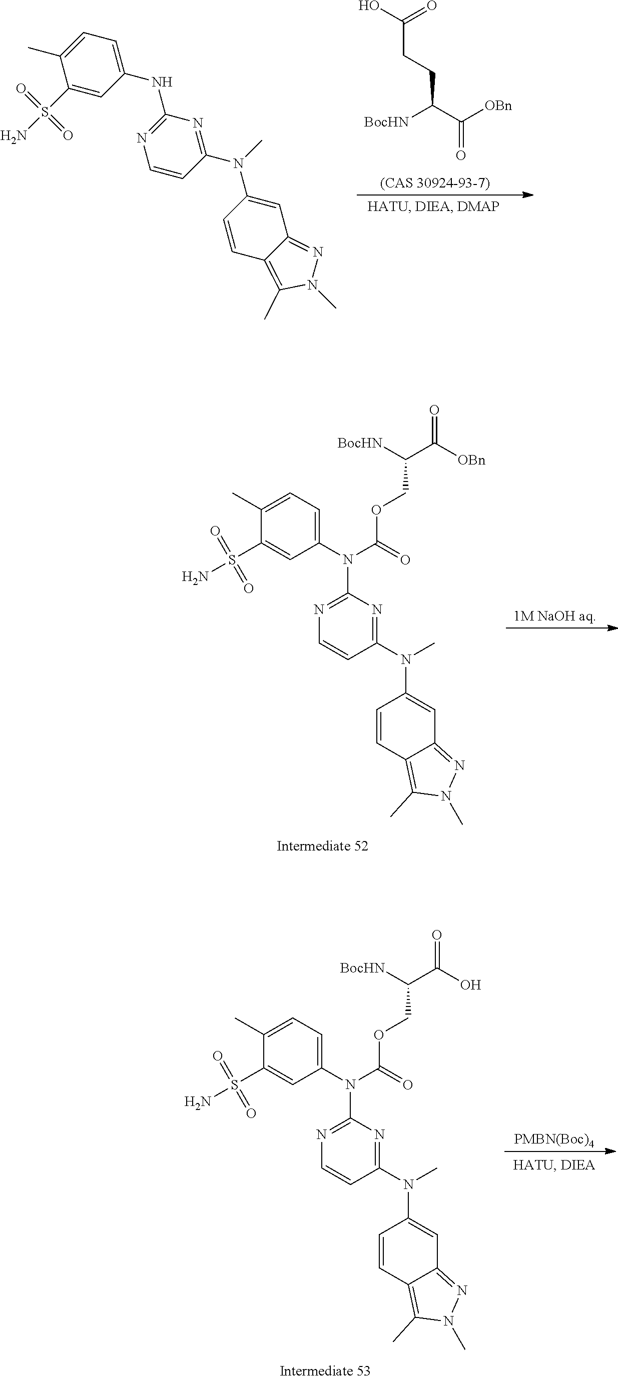 [SMILES: Cc1ccc(Nc2nccc(N(C)c3ccc4c(C)n(C)nc4c3)n2)cc1S(N)(=O)=O ; CC(C)(C)OC(=O)N[C@@H](CCC(=O)O)C(=O)OCc1ccccc1 ; Cc1ccc(N(C(=O)OC[C@H](NC(=O)OC(C)(C)C)C(=O)OCc2ccccc2)c2nccc(N(C)c3ccc4c(C)n(C)nc4c3)n2)cc1S(N)(=O)=O ; Cc1ccc(N(C(=O)OC[C@H](NC(=O)OC(C)(C)C)C(=O)O)c2nccc(N(C)c3ccc4c(C)n(C)nc4c3)n2)cc1S(N)(=O)=O ; "PMBN(Boc)4" ; "HATU, DIEA"]

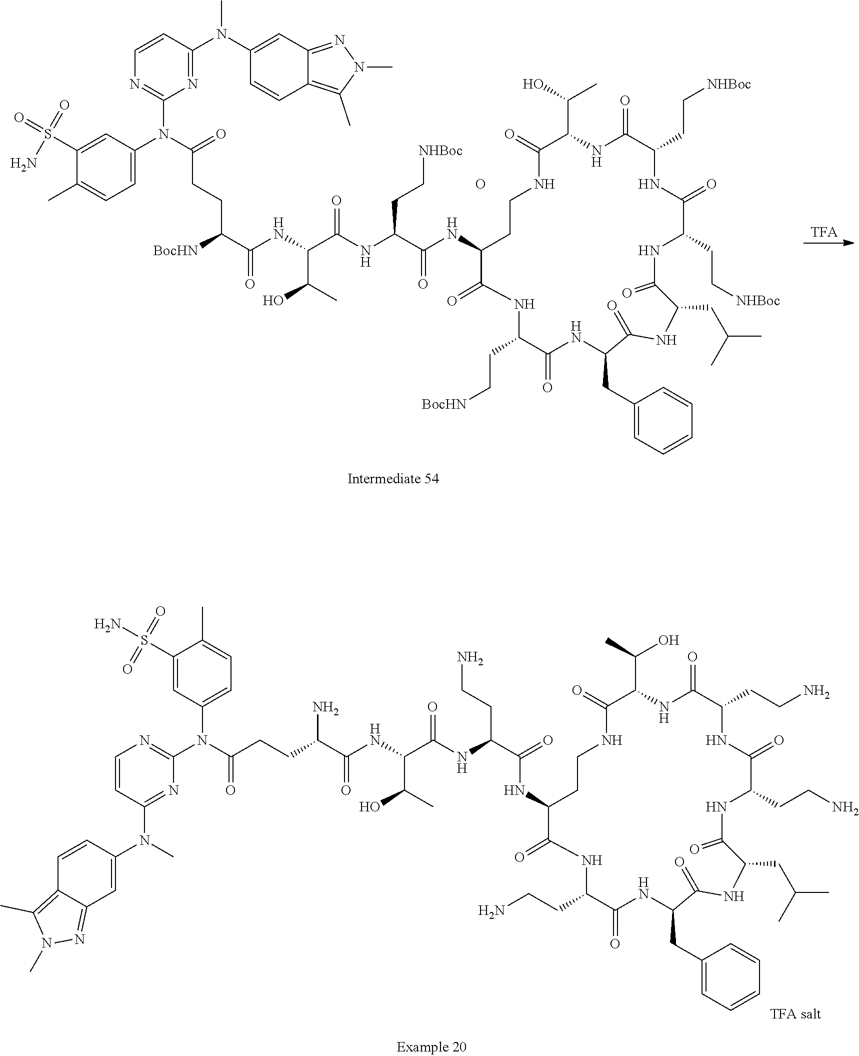

Intermediate 52. A 10 mL flask was charged with 5-((4-((2,3-dimethyl-2H-indazol-6-yl)(methyl)amino)pyrimidin-2-yl)amino)-2-methylbenzenesulfonamide (50.1 mg, 0.114 mmol), 30924-93-7 (42.5 mg, 0.126 mmol), HATU (65.3 mg, 1.50 eq.), DMAP (2.4 mg, cat.), DIEA (39.2 ul) and dry DMF (1 mL) under Ar. The reaction mixture was stirred at r.t. for 16 h, LC-MS showed the reaction was OK. The reaction mixture was diluted with EA, washed with 5% NaCl aq. sol. twice, and brine once, and then dried over anhydrous Na₂SO₄ and evaporated to give crude product. Purification by HPLC eluting with ACN/Water (0˜100% in 50 min, 15 mL/min) gave a white solid (80 mg).

Intermediate 53. A 10 mL flask was charged with Intermediate 52 (64.8 mg, 0.085 mmol), 1 M NaOH aq. sol. (0.428 mL, 5 eq.) and THF (1.2 mL) under Ar. The reaction mixture was stirred at r.t. for 16 h. The reaction mixture was acidified with 1M HCl aq. to pH 4-5, THE was removed under vacuum. The residue was purified by HPLC to give a white solid (15.7 mg).

Intermediate 54. A 10 mL flask was charged with Intermediate 53 (15.7 mg, 0.023 mmol), PMBN(Boc)₄ (35.3 mg, 0.025 mmol), HATU (9.8 mg, 0.025 mmol), DIEA (8 ul, 0.047 mmol) and dry DMF (1 mL) under Ar. The reaction mixture was stirred at r.t. for 16 h. The mixture was worked up with EA/H₂O/brine and then purified by HPLC eluting with ACN/Water (0˜100% in 50 min, 10 mL/min) to give a white solid (25.8 mg).

Compound of Example 20. A 10 mL flask was charged with Intermediate 54 (25.8 mg, 1.0 eq.), TFA (0.2 mL) and DCE (1 mL) under Ar. The reaction mixture was stirred at r.t. for 2˜3 h. Volatiles were removed under vacuum, then dried under vacuum to give crude product. Purification by HPLC eluting with ACN/Water (0˜100% in 60 min, 10 mL/min) gave a white solid (11.3 mg). MS: 1512.7 [M+H]⁺.

### Example 21

### Synthesis of the Compound of Example 21

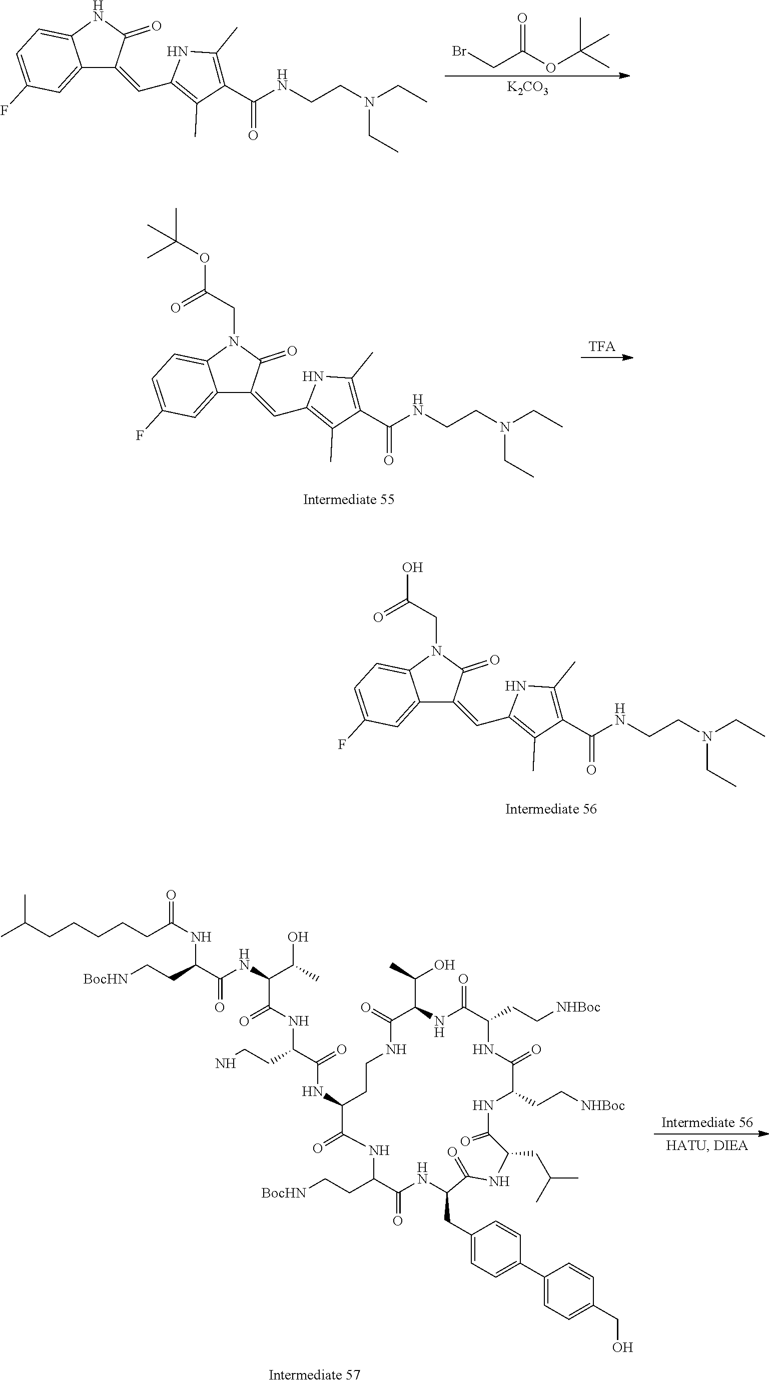

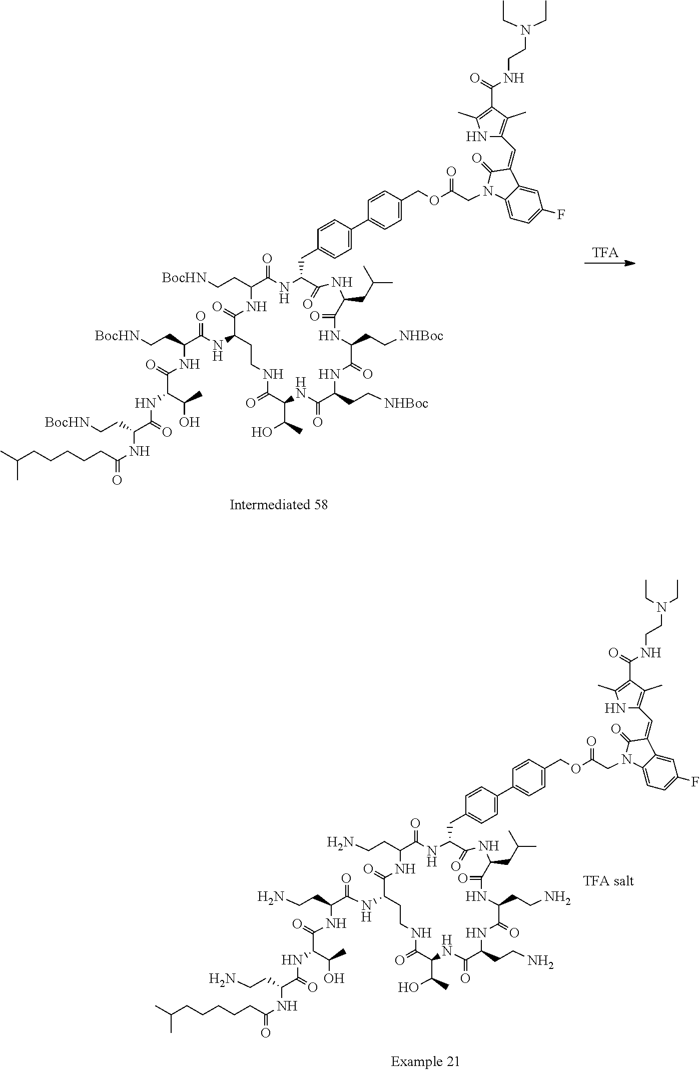

Intermediate 55. The mixture of (Z)—N-(2-(diethyl amino)ethyl)-5-((5-fluoro-2-oxoindolin-3-ylidene)methyl)-2,4-dimethyl-1H-pyrrole-3-carboxamide (200 mg, 0.5 mmol), t-butyl 2-bromoacetate (96 mg, 0.5 mmol) and K₂CO₃ (69 mg, 0.5 mmol) in DMF (2 mL) was stirred at r.t. for 7 h. The mixture was extracted with EA (30 mL), washed with H₂O (5 mL×2) and brine (5 mL). The EA layer was dried and evaporated. Purification by silica chromatograph (EA/PE=0˜100%) gave 0.2 g Intermediate 55.

Intermediate 56. The mixture of Intermediate 55 (0.2 g, 0.44 mmol) in TFA/DCM (0.6/5 mL) was stirred at r.t, for 1.5 h. The volatiles were removed to give crude 200 mg of Intermediate 56. MS: 457.2 (M+1).

Intermediate 58. The mixture of Intermediate 57 (330 mg, 0.18 mmol), Intermediate 56 (102 mg, 0.22 mmol), HATU (104 mg, 0.27 mol) and DIEA (0.082 mL, 0.46 mmol) in DMF (3 mL) was stirred under Ar at r.t. for 18 h. The mixture was extracted with EA (30 mL), washed with H₂O (5 mL×2) and brine (5 mL). The EA layer was dried and evaporated. Purification by silica chromatography (MeOH/DCM=0˜15%) gave 0.17 g of Intermediate 58. MS: 1124.6 [M+2H]²⁺.

Compound of Example 21. The mixture of Intermediate 58 (0.2 g, 0.089 mmol) in TFA/DCM (1.2/5 mL) was stirred at r.t for 1.5 h. The volatiles were removed, and the residue was purified by C18 chromatography to give 68 mg of the Compound of Example 21. NMR: 7.42 (s, 4H), 7.21 (d, J=38.0 Hz, 6H), 6.79 (d, J=28.4 Hz, 4H), 5.22-5.10 (m, 2H), 4.62 (d, J=13.0 Hz, 5H), 4.50 (d, J=6.8 Hz, 2H), 4.42 (dd, J=9.6, 5.6 Hz, 5H), 4.30 (d, J=4.2 Hz, 2H), 4.23-4.10 (m, 9H), 3.36-3.19 (m, 11H), 3.02 (s, 21H), 2.29-1.73 (m, 31H), 1.48 (s, 3H), 1.27 (t, J=7.3 Hz, 11H), 1.24-0.92 (m, 22H), 0.79-0.60 (m, 12H), 0.34 (d, J=16.3 Hz, 5H). MS: 1770.7 [M+Na]⁺.

### Example 22

### Synthesis of the Compound of Example 22

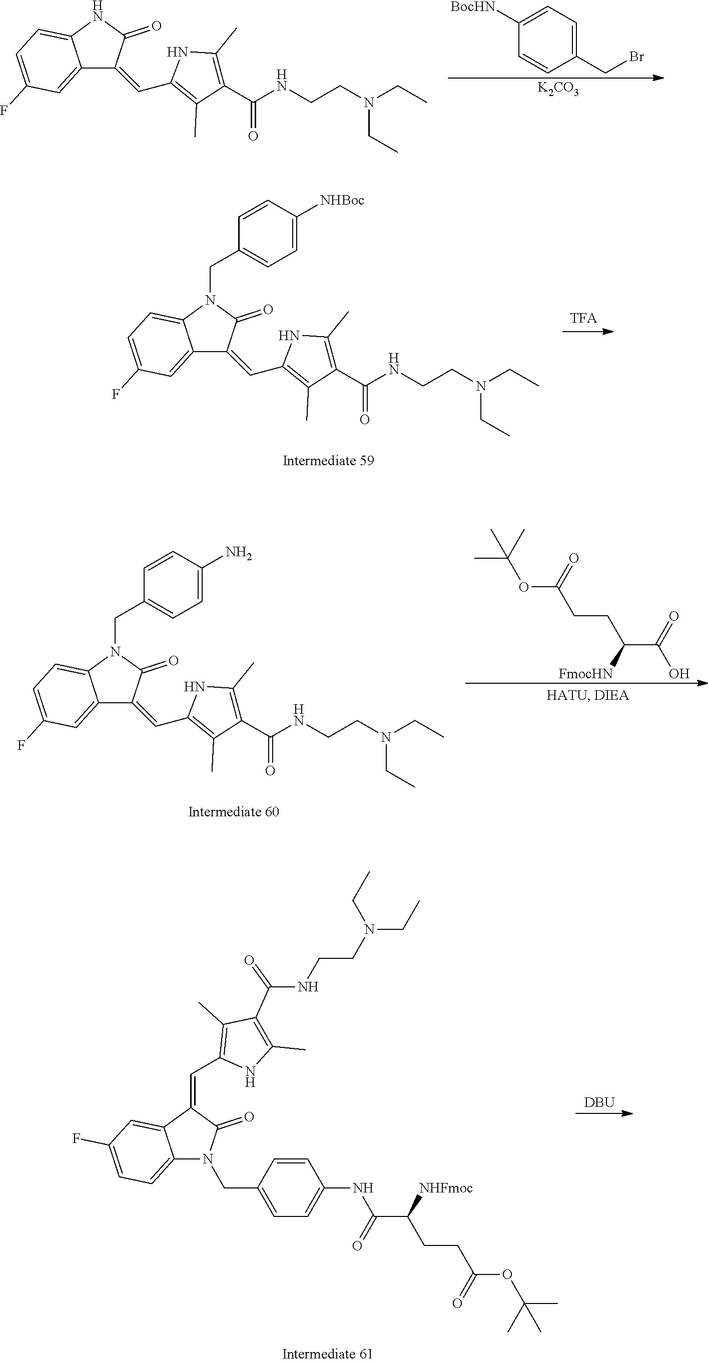

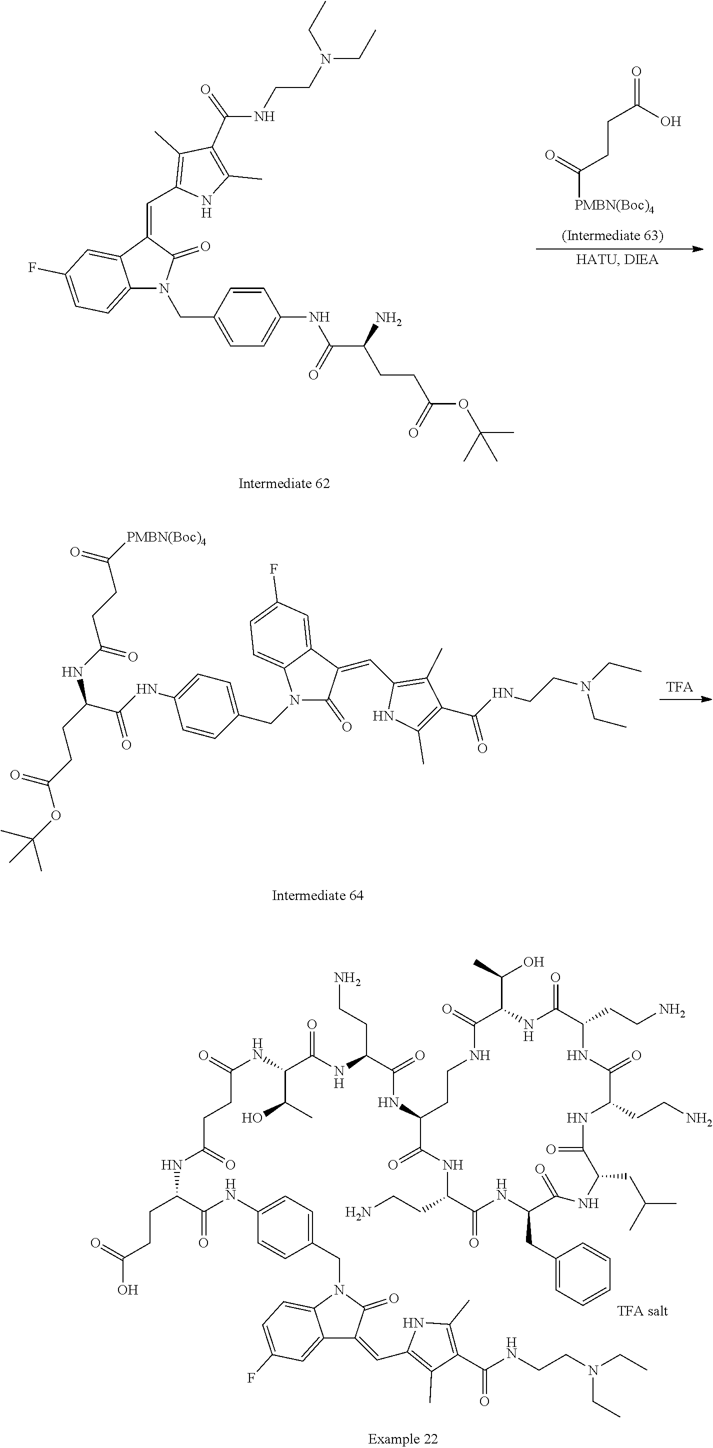

Intermediate 59. The mixture of (Z)—N-(2-(diethylamino)ethyl)-5-((5-fluoro-2-oxoindolin-3-ylidene)methyl)-2,4-dimethyl-1H-pyrrole-3-carboxamide (200 mg, 0.5 mmol), t-butyl (4-(bromomethyl)phenyl)carbamate (157 mg, 0.55 mmol) and K₂CO₃ (70 mg, 0.5 mmol) in DMF (5 mL) was stirred at r.t. o.n. The mixture was extracted with EA (30 mL), washed with H₂O (5 mL×2) and brine (5 mL). The EA layer was dried and evaporated. Purification by silica chromatography (MeOH/DCM=0˜15%) gave 0.3 g of Intermediate 59. MS: 604.29 (M+1).

Intermediate 60. The mixture of Intermediate 59 (0.3 g, 0.6 mmol) in TFA/DCM (0.3/2 mL) was stirred at r.t for 1.5 h. The volatiles were removed and the residue was washed with MTBE (1 mL×2) to give 260 mg of crude Intermediate 60. MS: 504.18 (M+1).

Intermediate 61. The mixture of Intermediate 60 (380 mg, 0.75 mmol), (S)-2-((((9H-fluoren-9-yl)methoxy)carbonyl)amino)-5-(tert-butoxy)-5-oxopentanoic acid (380 mg, 0.9 mmol), HATU (430 mg, 1.13 mmol) and DIEA (0.27 mL, 1.5 mmol) in DMF (5 mL) was stirred under Ar at r.t. o.n. The mixture was extracted with EA (40 mL), washed with H₂O (5 mL×2) and brine (5 mL). The EA layer was dried and evaporated. Purification by silica chromatography (MeOH/DCM=0˜15%) gave 0.5 g of Intermediate 61. MS: 911.38 (M+1).

Intermediate 62. The mixture of Intermediate 61 (0.6 g, 0.66 mmol), DBU (0.2 mL) in NMP (5 mL) was stirred under Ar at r.t. for 2.5 h. The mixture was extracted with EA (40 mL), washed with H₂O (5 mL×2) and brine (5 mL). The EA layer was dried and evaporated. Purification by C18 chromatography (ACN/H₂O=0˜100%) gave 0.37 g of Intermediate 62. LC/MS: 689.28 (M+1).

Intermediate 64. The mixture of Intermediate 62 (300 mg, 0.44 mmol), Intermediate 63 (420 mg, 0.29 mmol), HATU (121 mg, 0.32 mmol) and DIEA (0.078 mL, 0.44 mmol)) in DMF (5 mL) was stirred under Ar at r.t. o.n. The mixture was extracted with EA (40 mL), washed with H₂O (5 mL×2) and brine (5 mL). The EA layer was dried and evaporated. Purification by silica chromatography (MeOH/DCM=0˜15%) gave 0.44 g of Intermediate 64. LC/MS: 1067.61 (½ M+1).

Compound of Example 22. The mixture of Intermediate 64 (0.45 g, 0.21 mmol) in TFA/DCM (0.5 mL/5 mL) was stirred at r.t for 4.5 h. The volatiles were removed and the residue was purified by C18 chromatography (ACN/H₂O=0˜60%) to give 142 mg of the Compound of Example 22. NMR: 7.27 (d, J=8.2 Hz, 2H), 7.16 (d, J=8.0 Hz, 2H), 7.05 (s, 3H), 6.98 (s, 3H), 6.62 (s, 2H), 4.31 (s, 4H), 4.18-3.92 (m, 9H), 3.62 (s, 2H), 3.30-3.11 (m, 7H), 3.02-2.80 (m, 9H), 2.69 (s, 4H), 2.51 (s, 5H), 2.36 (s, 3H), 2.24-1.46 (m, 22H), 1.29 (s, 2H), 1.21 (t, J=7.2 Hz, 7H), 1.04 (d, J=6.3 Hz, 3H), 0.95 (d, J=5.6 Hz, 3H), 0.60 (d, J=6.7 Hz, 3H), 0.53 (d, J=6.7 Hz, 3H). MS: 1677.7 [M+H]⁺.

### Example 23

### Synthesis of the Compound of Example 23

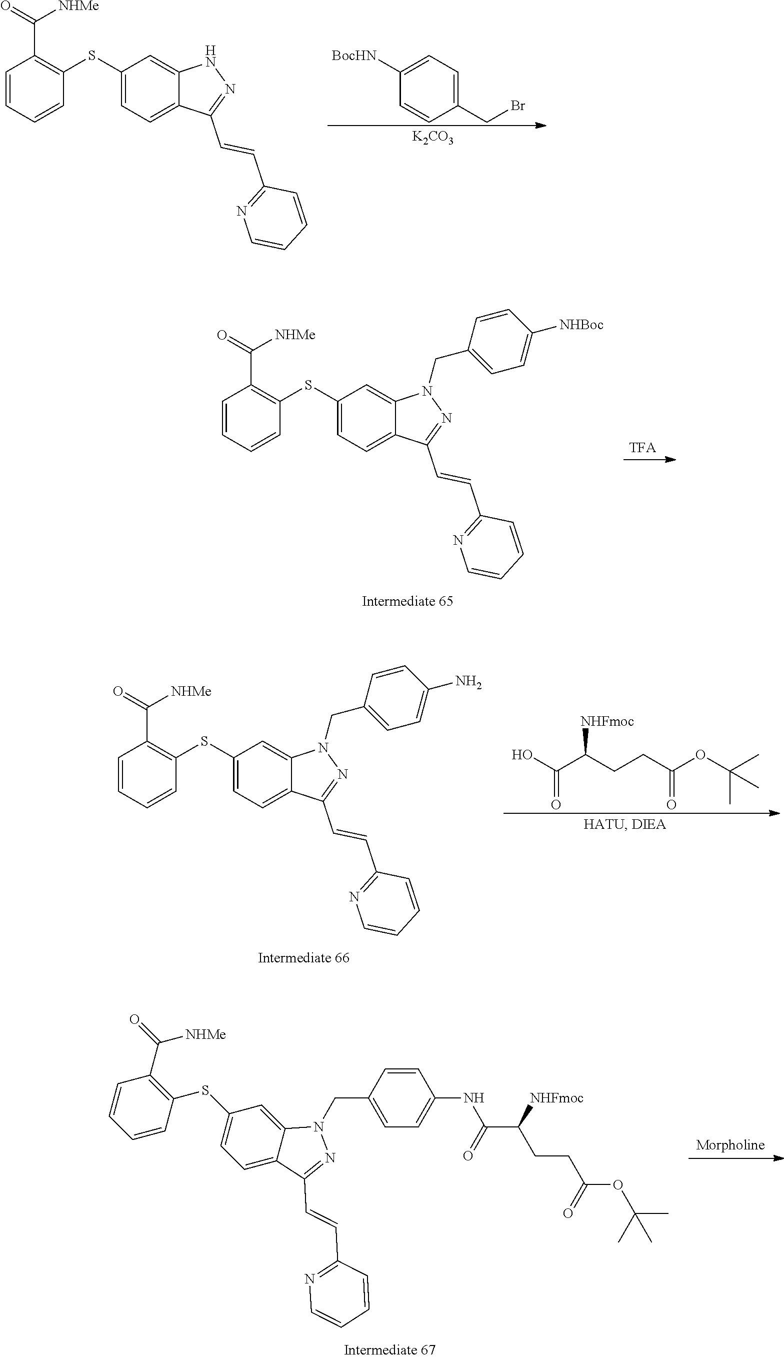

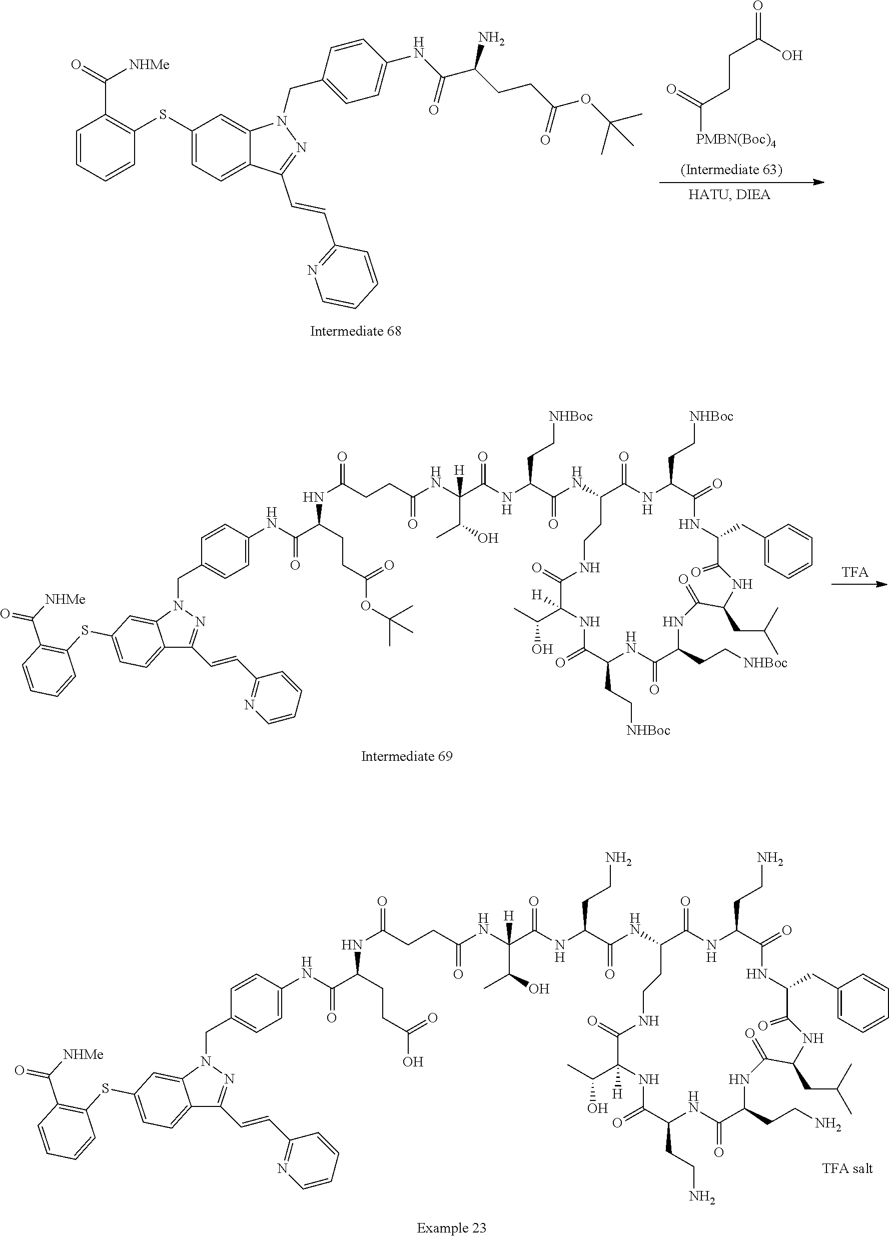

Intermediate 65. To a solution of (E)-N-methyl-2-((3-(2-(pyridin-2-yl)vinyl)-1H-indazol-6-yl)thio)benzamide (488 mg, 1.3 mmol), K₂CO₃ (897 mg, 6.5 mmol) and t-butyl (4-(bromomethyl)phenyl)carbamate (744 mg, 2.6 mmol) in 15 mL of DMF was stirred at 95° C. for 24 h. The reaction mixture was diluted with 250 mL of EA, and then washed with H₂O (30 mL×2), and brine (30 mL×2), and then the EA layer was dried and evaporated. The crude product was purified by HPLC to give 218 mg of Intermediate 65 and some regioisomer Intermediate 70. MS: 592.2 [M+H]⁺.

Intermediate 66. A mixture of Intermediate 65 (114 mg, 0.19 mmol) in 1 mL/5 mL of TFA/DCM was stirred at r.t. o.n. for 6 h. Volatiles were evaporated, the crude product was purified by HPLC to give 132 mg of Intermediate 66. MS: 492.1 [M+H]⁺.

Intermediate 67. A mixture of Intermediate 66 (110 mg, 0.2 mmol), (S)-2-((((9H-fluoren-9-yl)methoxy)carbonyl)amino)-5-(tert-butoxy)-5-oxopentanoic acid (130 mg, 0.3 mmol), DIEA (96 μL, 0.5 mmol), HATU (155 mg, 0.4 mmol) in 15 mL of DMF was stirred at r.t. for 16 h. The reaction mixture was diluted with 150 mL of EA, and then washed with H₂O (20 mL×2), and brine (20 mL×2), and then the EA layer was dried and evaporated. The crude product was purified by HPLC to give 168 mg of Intermediate 67. MS: 899.8 [M+H]⁺.

Intermediate 68. A mixture of crude Intermediate 67 (102 mg, 0.11 mmol) in 0.5 mL/10 mL of morpholine/DCM was stirred at r.t. for 6 h. Volatiles were evaporated, diluted with 100 mL of EA, and then washed with H₂O (20 mL×2), and brine (20 mL×2), and the EA layer was dried and evaporated. The crude product was purified by HPLC to give 58 mg. MS: 677.2 [M+H]⁺.

Intermediate 69. A mixture of Intermediate 68 (81 mg, 0.12 mmol), Intermediate 63 (140 mg, 0.1 mmol), TEA (40 μL, 0.25 mmol), HATU (57 mg, 0.15 mmol) in 10 mL of DMF was stirred at r.t. for 6 h. The reaction mixture was diluted with 150 mL of EA, and then washed with H₂O (20 mL×2), and brine (20 mL×2), and the EA layer was dried and evaporated. The crude product was purified by HPLC to give 110 mg of Intermediate 69. MS: 1061.4 [M+2H⁺]/2.

Compound of Example 23. A mixture of Intermediate 69 (110 mg, 0.05 mmol) in 1 mL/5 mL of TFA/DCM was stirred at r.t. o.n. Volatiles were evaporated, and the crude product was purified by HPLC to give 83 mg of the Compound of Example 23. NMR: 8.54 (d, J 6.0 Hz, 1H), 8.42-8.35 (m, 1H), 8.13-8.04 (m, 1H), 7.91-7.71 (m, 2H), 7.41-6.91 (m, 17H), 5.71 (s, 1H), 5.41 (s, 1H), 4.53-4.08 (m, 14H), 3.18-2.38 (m, 23H), 2.34-1.70 (m, 14H), 1.42-1.36 (m, 2H), 1.16-1.03 (m, 6H), 0.78-0.56 (m, 8H). MS: 1665.5 [M+H]⁺.

### Example 24

### Synthesis of the Compound of Example 24

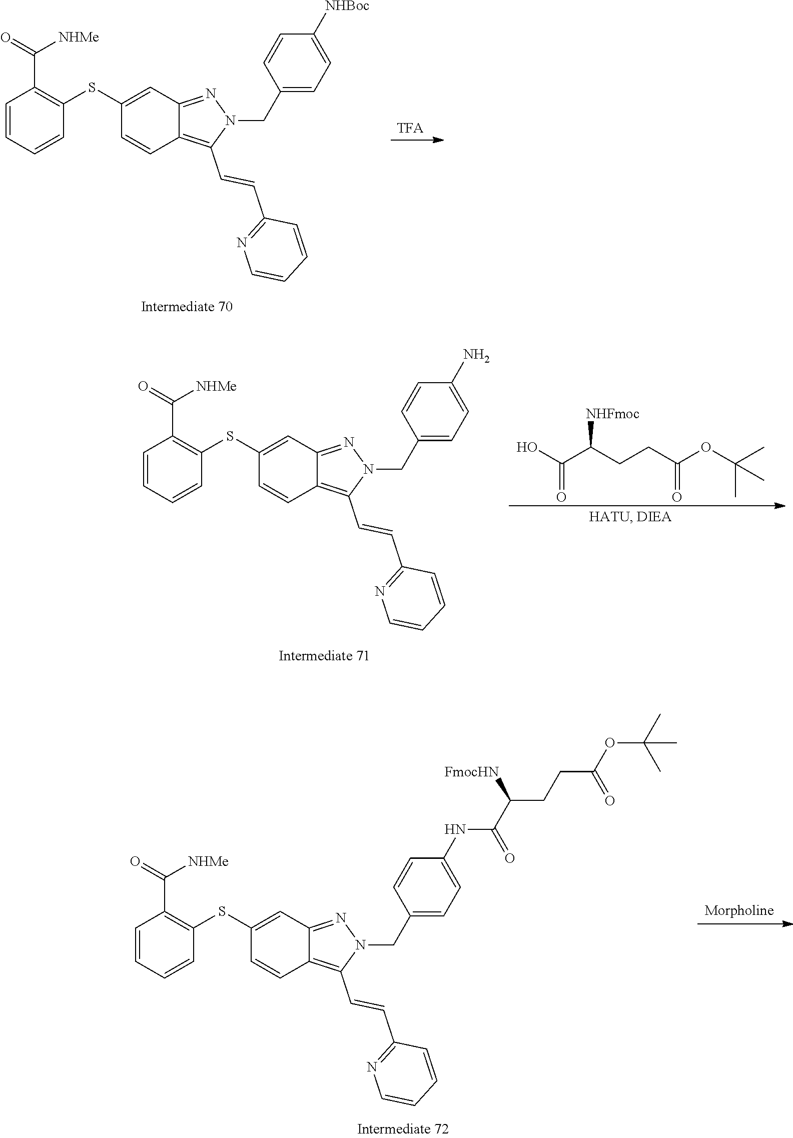

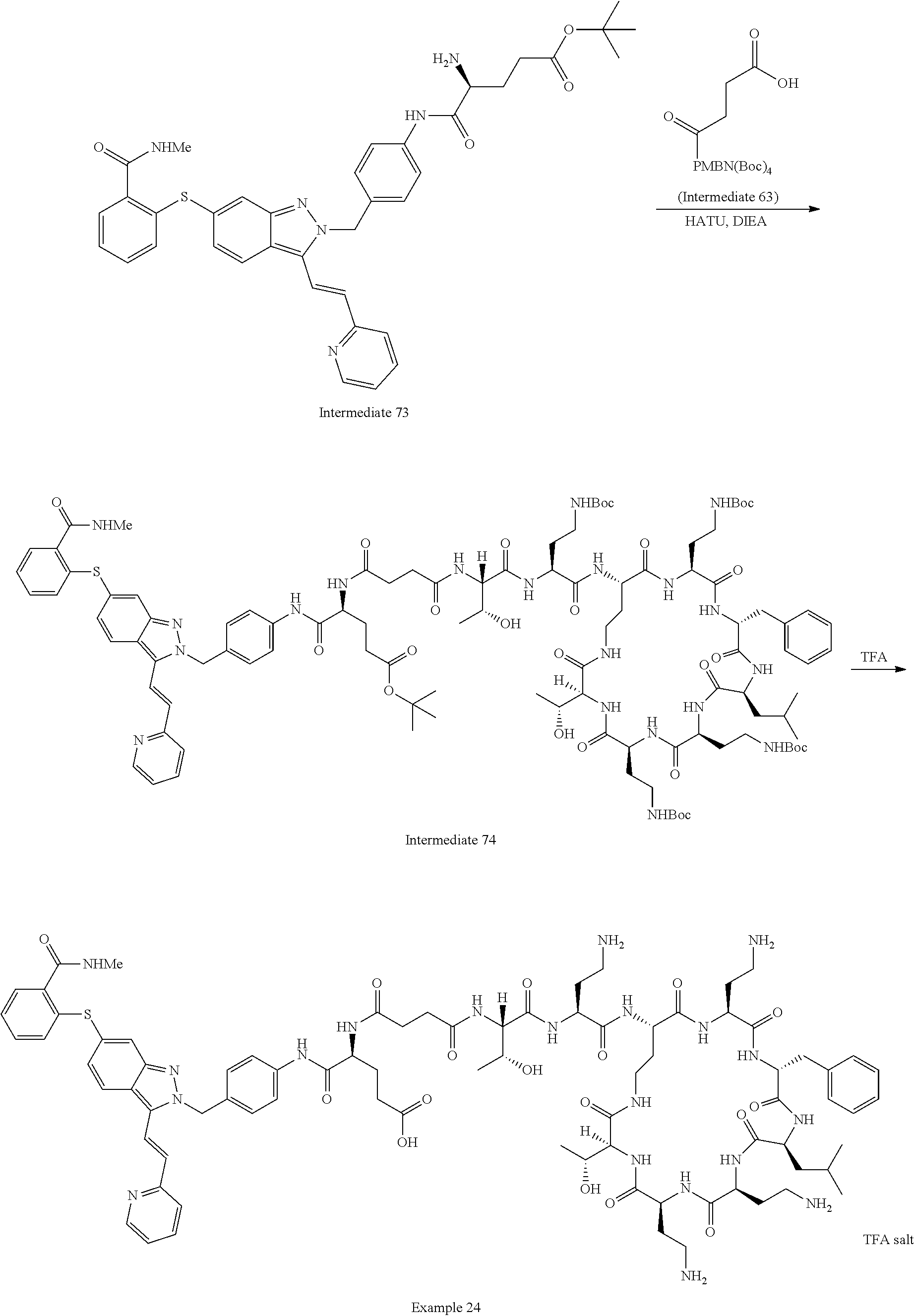

Compound of Example 24. The Compound of Example 24 was prepared according to procedures described for the synthesis of the compound of Example 23, except using Intermediate 70 (instead of Intermediate 65 used for the synthesis of the compound of Example 23) to afford 26 mg of the Compound of Example 24. NMR: 8.54 (d, J 2.8 Hz, 1H), 8.42-8.26 (m, 1H), 8.05-7.71 (m, 3H), 7.44-6.98 (m, 16H), 5.75 (s, 1H), 5.61 (s, 1H), 5.48 (s, 1H), 4.55-4.06 (m, 14H), 3.19-2.37 (m, 23H), 2.26-1.66 (m, 14H), 1.42-1.32 (m, 2H), 1.16-1.01 (m, 6H), 0.84-0.55 (m, 8H). MS: 1665.6 [M+H]⁺.

### Example 25

### Synthesis of the Compound of Example 25

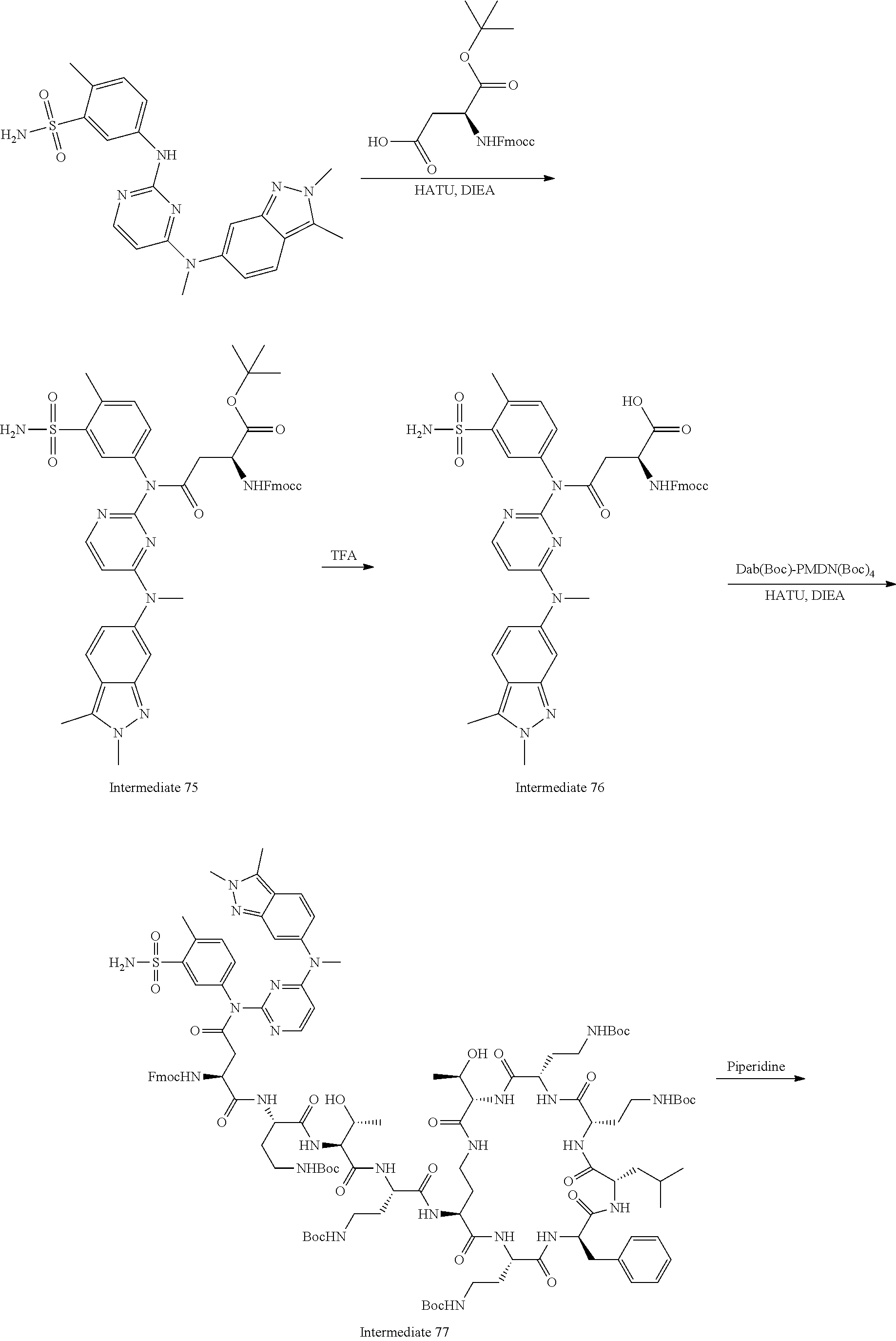

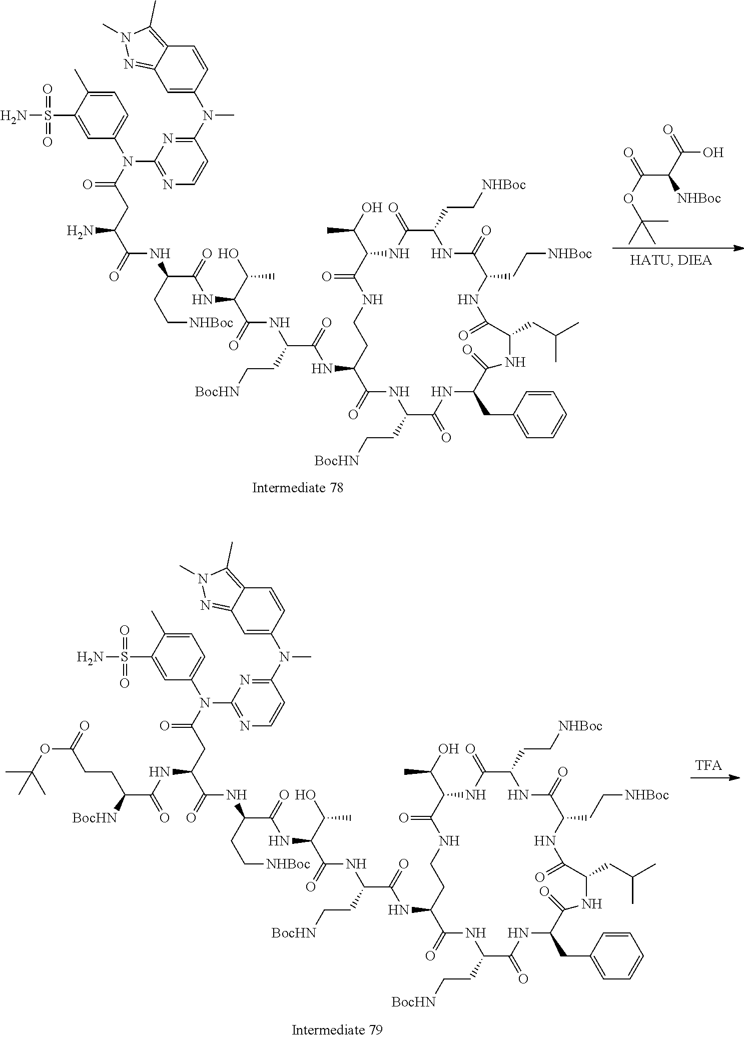

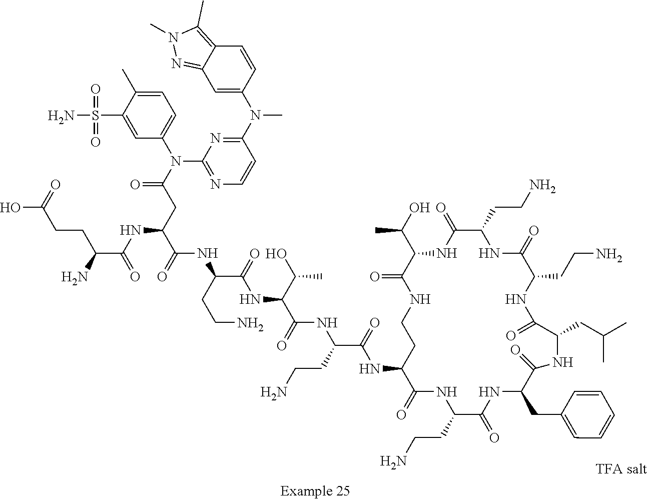 [SMILES: Cc1ccc(N(C(=O)C[C@H](NC(=O)[C@@H](N)CCC(=O)O)C(=O)N[C@H](CCN)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)c2nccc(N(C)c3ccc4c(C)n(C)nc4c3)n2)cc1S(N)(=O)=O]

Intermediate 75. To a solution of 5-((4-((2,3-dimethyl-2H-indazol-6-yl)(methyl)amino)pyrimidin-2-yl)amino)-2-methylbenzenesulfonamide (385 mg, 0.88 mmol), (S)-3-((((9H-fluoren-9-yl)methoxy)carbonyl)amino)-4-(tert-butoxy)-4-oxobutanoic acid (724 mg, 1.76 mmol), DIEA (471 μL, 2.64 mmol), and HATU (836.4 mg, 2.2 mmol) in 15 mL of DMF was stirred at r.t. for 6 h. The reaction mixture was diluted with 250 mL of EA, and then washed with H₂O (20 mL×2), and brine (20 mL×2), and the EA layer was dried and evaporated. The crude product was purified by HPLC to give 296 mg of Intermediate 75. MS: 831.4 [M+H]⁺.

Intermediate 76. A mixture of Intermediate 75 (190 mg, 0.23 mmol) in 1 mL/5 mL of TFA/DCM was stirred at r.t. o.n. for 6 h. Volatiles were evaporated, diluted with 150 mL of EA, and then washed with H₂O (20 mL×2), and brine (20 mL×2), and the EA layer was dried and evaporated. The crude product was purified by HPLC to give 142 mg of Intermediate 76. MS: 775.6 [M+H]⁺.

Intermediate 77. A mixture of Intermediate 76 (55 mg, 0.07 mmol), Dab(Boc)PMBN(Boc)₄ (112 mg, 0.07 mmol), DIEA (18 μL, 0.14 mmol), HATU (30 mg, 0.08 mmol) in 5 mL of DMF was stirred at r.t. for 4 h. The reaction mixture was diluted with 100 mL of EA, and then washed with H₂O (20 mL×2), and brine (20 mL×2), and the EA layer was dried and evaporated. The crude product was purified by silica gel chromatography to give 118 mg of Intermediate 77. MS: 1161.1 [½M+H]⁺.

Intermediate 78. A mixture of Intermediate 77 (285 mg, 0.12 mmol), piperidine (51 mg, 0.60 mmol), in 10 mL of DMF was stirred at r.t. for 2 h. Volatiles were evaporated, diluted with 200 mL of EA, and then washed with H₂O (20 mL×2), and brine (20 mL×2), and the EA layer was dried and evaporated. The crude product was purified by HPLC to give 205 mg of Intermediate 78. LCMS: 1049.6 [M+2H⁺]/2.

Intermediate 79. A mixture of Intermediate 78 (105 mg, 0.05 mmol), SM2 (18 mg, 0.06 mmol), DIEA (15 μL, 0.08 mmol), HATU (27 mg, 0.07 mmol) in 6 mL of DMF was stirred at r.t. for 16 h. The reaction mixture was diluted with 50 mL of EA, and then washed with H₂O (10 mL×2), and brine (10 mL×2), and the EA layer was dried and evaporated. The crude product was purified by HPLC to give 103 mg of Intermediate 79. MS: 1192.2 [M+H]⁺.

Compound of Example 25. A mixture of Intermediate 79 (103 mg, 0.04 mmol) in 0.5 mL/5 mL of TFA/DCM was stirred at r.t. for 6 h. Volatiles were evaporated, the crude product was purified by HPLC to give 22 mg of the Compound of Example 25. NMR: 8.02 (brd s, 1H), 8.02 (brd s, 2H), 7.36-7.08 (m, 11H), 6.86-6.54 (m, 2H), 4.56-4.41 (m, 7H), 4.31-3.96 (m, 19H), 3.39-2.96 (m, 24H), 2.90-2.64 (m, 10H), 2.50-1.80 (m, 29H), 1.46-1.04 (m, 12H), 0.78-0.66 (m, 9H). MS: 1727.5 [M+H]⁺.

### Example 26. Synthesis of the Compound of Example 26

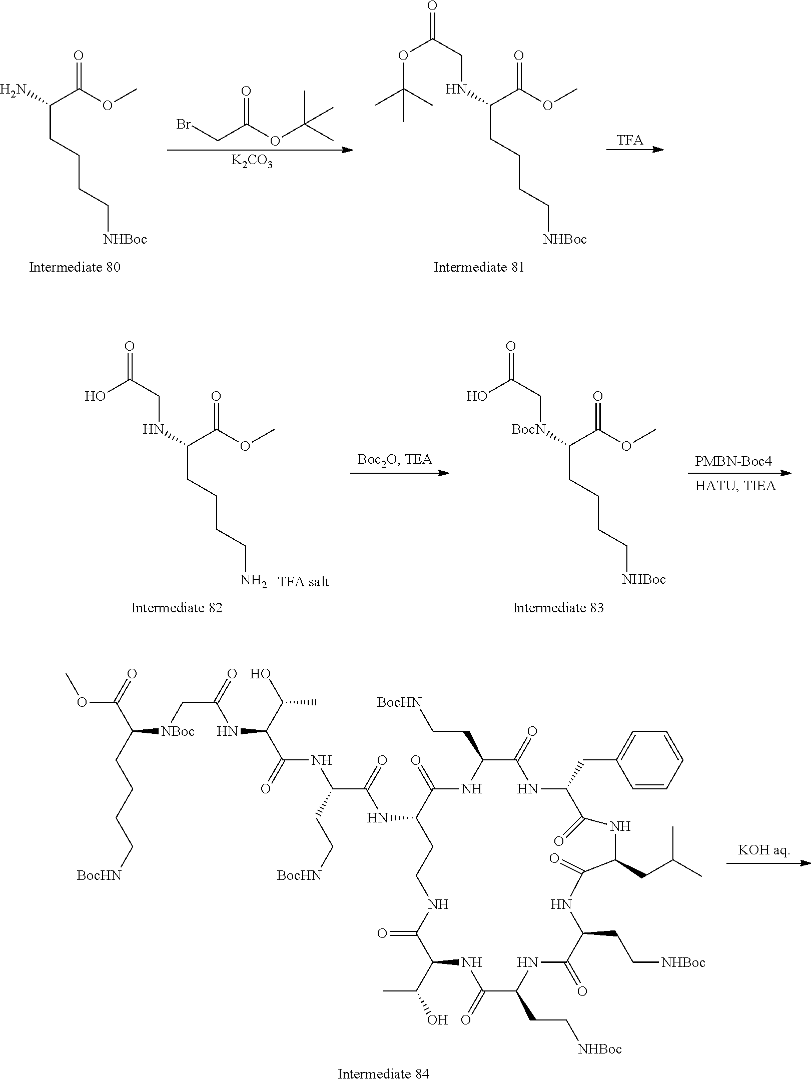

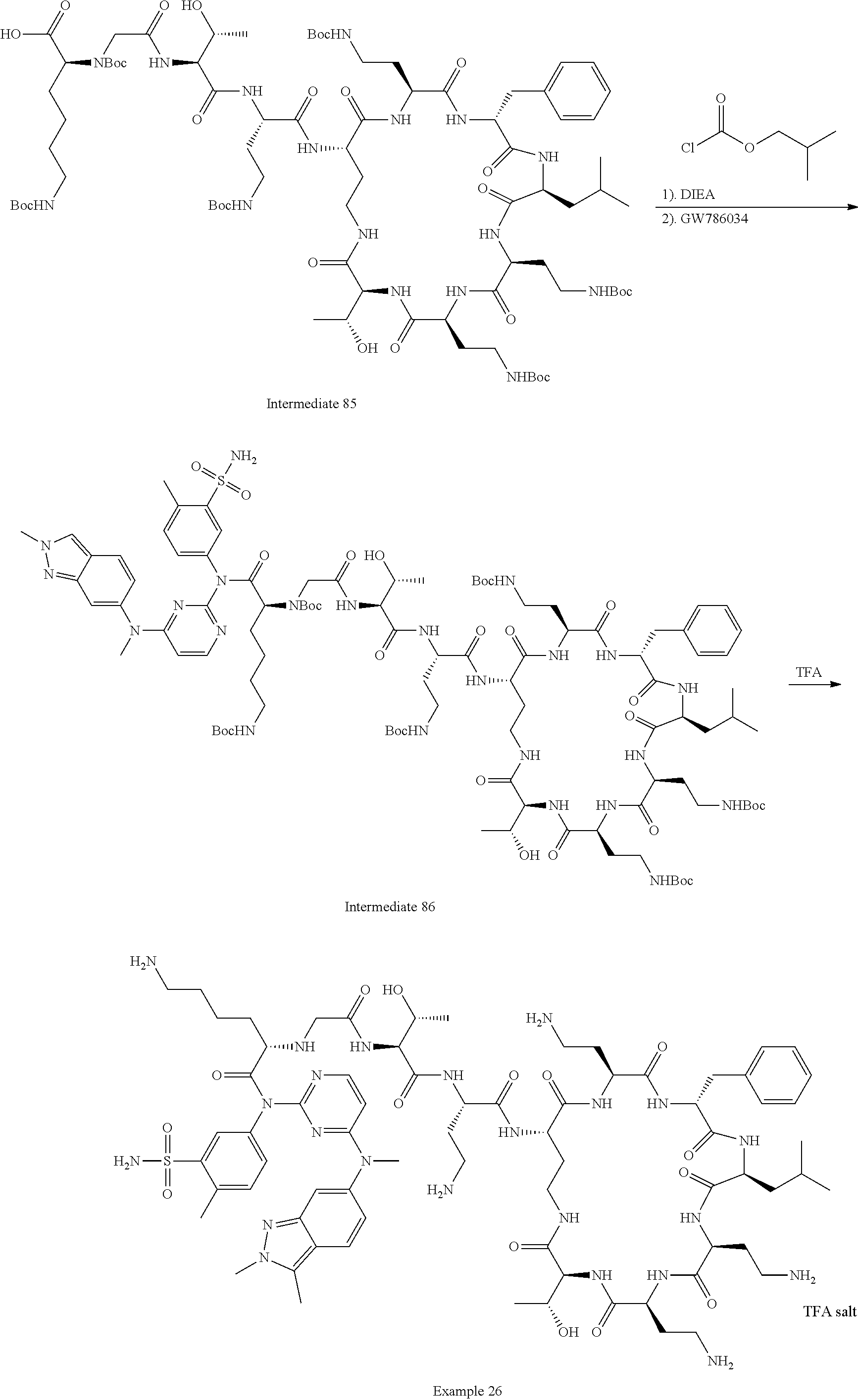

Intermediate 81. To a solution of tert-butyl 2-bromoacetate (2.2 g, 7.4 mmol), K₂CO₃ (2.6 g, 18.5 mmol) and (S)-methyl 2-amino-6-((tert-butoxycarbonyl)amino)hexanoate (Intermediate 80) (1.4 g, 7.4 mmol) in 20 mL of ACN was stirred at 45° C. for 6 h. The reaction mixture was diluted with 250 mL of EA, and then washed with H₂O (30 mL×2), and brine (30 mL×2), and the EA layer was dried and evaporated. The crude product was purified by silica gel chromatography to give 2.3 g of Intermediate 81. MS: 375.1 [M+H]⁺.

Intermediate 82. A mixture of Intermediate 81 (1.1 g, 2.8 mmol) in 2.5 mL/10 mL of TFA/DCM was stirred at r.t. o.n. for 6 h. Volatiles were evaporated, and the crude product was purified by HPLC to give 558 mg of Intermediate 82. MS: 219.2 [M+H]⁺.

Intermediate 83. To a solution of Intermediate 82 (550 mg, 2.5 mmol), Boc₂O (1.6 g, 7.5 mmol) and TEA (2.2 mL, 15.0 mmol) in 30 mL of THF was stirred at r.t. for 6 h. The reaction mixture was diluted with 250 mL of EA, and then washed with H₂O (30 mL×2), and brine (30 mL×2), and the EA layer was dried and evaporated. The crude product was purified by silica gel chromatography to give 388 mg of Intermediate 83. MS: 419.2 [M+H]⁺.

Intermediate 84. A mixture of Intermediate 83 (117 mg, 0.28 mmol), PMBN(Boc)₄ (388 mg, 0.28 mmol), DIEA (77 μL, 0.42 mmol), HATU (130 mg, 0.34 mmol) in 12 mL of DMF was stirred at r.t. for 3 h. The reaction mixture was diluted with 150 mL of EA, and then washed with H₂O (20 mL×2), and brine (20 mL×2), and the EA layer was dried and evaporated. The crude product was purified by HPLC to give 369 mg of Intermediate 84. MS: 1763.4 [M+H]⁺.

Intermediate 85. A mixture of Intermediate 84 (386 mg, 0.22 mmol), KOH (18 mg, 0.33 mmol) in 5 mL/5 mL of H₂O/THF was stirred at r.t. for 2 h. The reaction was quenched with 0.1 M HCl, volatiles evaporated, and then product extracted with 200 mL of EA. The EA layer was washed with H₂O (20 mL×2), and brine (20 mL×2), and dried and volatiles evaporated. The crude product was purified by HPLC to give 209 mg of Intermediate 5. MS: 1749.4 [M+H]⁺.

Intermediate 86. A mixture of Intermediate 85 (150 mg, 0.09 mmol), isobutyl chloroformate (15 mg, 0.1 mmol), DIEA (30 μL, 0.18 mmol) in 10 mL of THF was stirred at r.t. for 2 h. The reaction mixture was diluted with 50 mL of EA, and then washed with H₂O (10 mL×2), and brine (10 mL×2), and the EA layer was dried and evaporated to give a crude solid, used directly. To a solution of 5-((4-((2,3-dimethyl-2H-indazol-6-yl)(methyl)amino)pyrimidin-2-yl)amino)-2-methylbenzenesulfonamide (78 mg, 0.18 mmol) in 2.5 mL of DMF was added NaH (9 mg, 0.23 mmol) at 0° C. Then the crude product was added into the mixture, and stirred at r.t. for 24 h. The reaction mixture was diluted with 100 mL of EA, and then washed with H₂O (20 mL×2), and brine (20 mL×2), and the EA layer was dried and evaporated. The crude product was purified by HPLC to give 65 mg of Intermediate 86. LCMS: 1085.1 [½M+H]⁺.

Compound of Example 26. A mixture of Intermediate 86 (60 mg, 0.03 mmol) in 0.1 mL/0.5 mL of TFA/DCM was stirred at r.t. for 4 h. Volatiles were evaporated, and the crude product was purified by HPLC to give 25 mg of the Compound of Example 26. NMR: 7.85 (d like, J 5.2 Hz, 1H), 7.65-7.18 (m, 12H), 6.97 (d, J 7.6 Hz, 1H), 4.54-4.36 (m, 5H), 4.26-3.89 (m, 15H), 3.70-3.68 (m, 1H), 3.56-3.49 (m, 3H), 3.31-3.26 (m, 1H), 3.08-2.47 (m, 25H), 2.21-1.60 (m, 19H), 1.48-1.04 (m, 14H), 0.74-0.49 (m, 10H). MS: 1568.5 [M+H]⁺.

### Example 27

### Synthesis of the Compound of Example 27

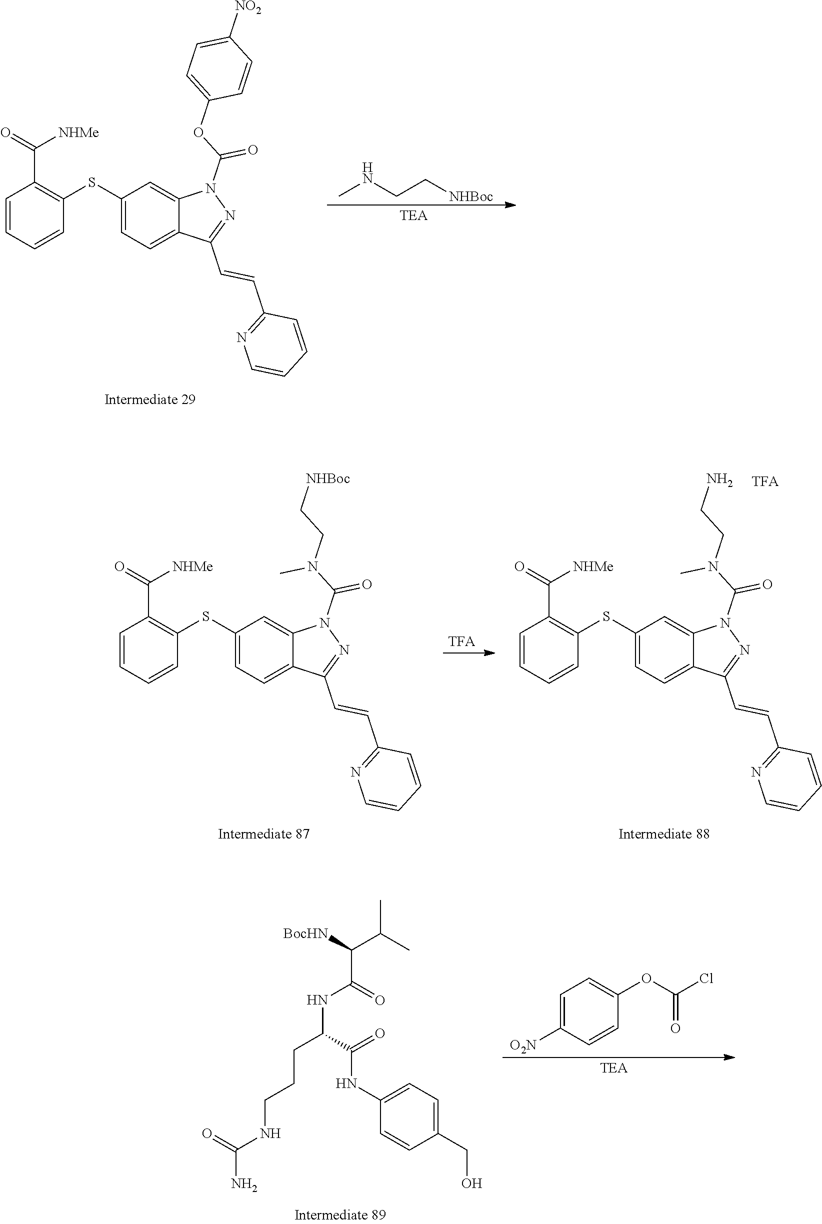 [SMILES: CNC(=O)c1ccccc1Sc1ccc2c(/C=C/c3ccccn3)nn(C(=O)Oc3ccc([N+](=O)[O-])cc3)c2c1 ; CNCCNC(=O)OC(C)(C)C ; CNC(=O)c1ccccc1Sc1ccc2c(/C=C/c3ccccn3)nn(C(=O)N(C)CCNC(=O)OC(C)(C)C)c2c1 ; CNC(=O)c1ccccc1Sc1ccc2c(/C=C/c3ccccn3)nn(C(=O)N(C)CCN)c2c1 ; CC(C)[C@H](NC(=O)OC(C)(C)C)C(=O)N[C@@H](CCCNC(N)=O)C(=O)Nc1ccc(CO)cc1 ; O=C(Cl)Oc1ccc([N+](=O)[O-])cc1]

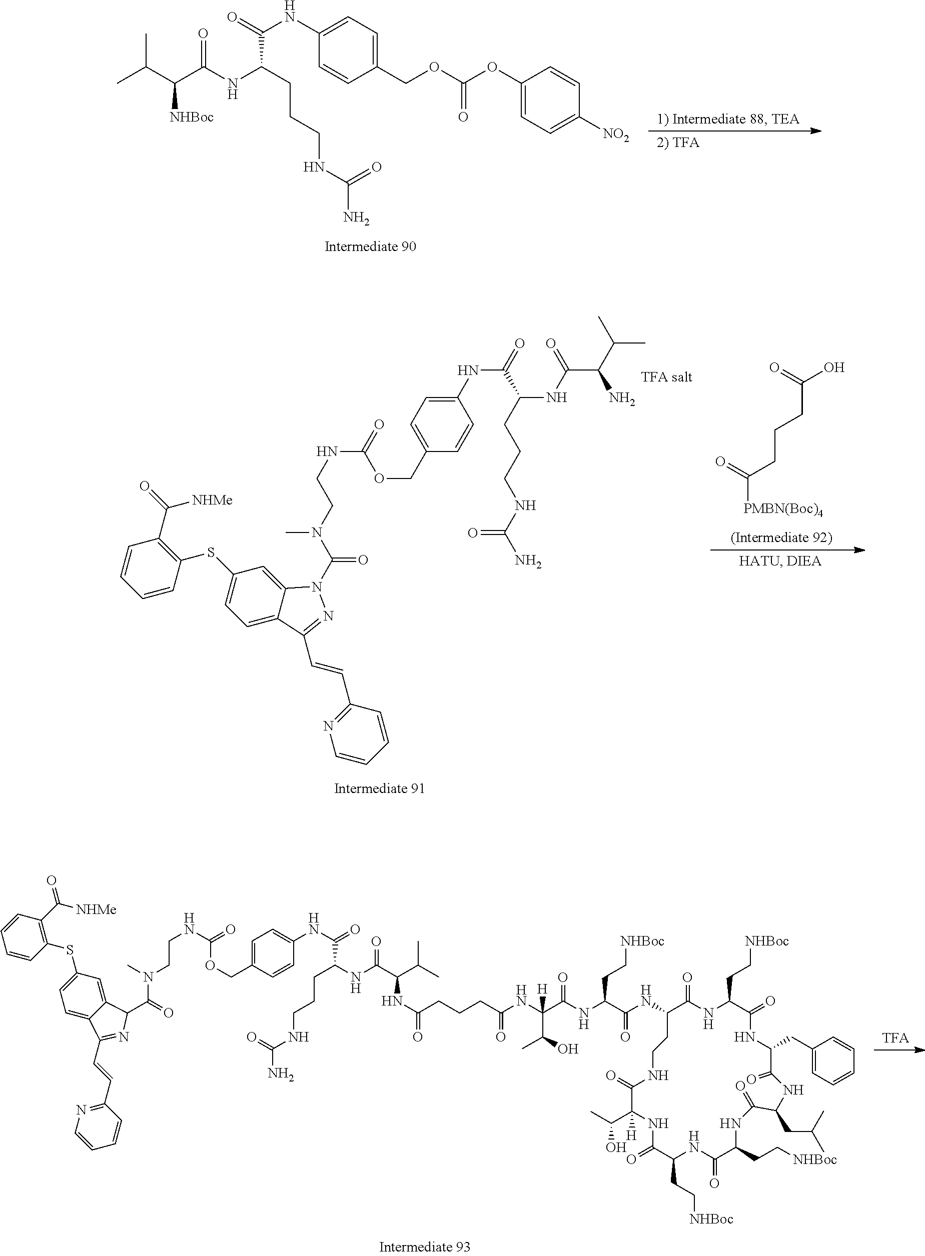

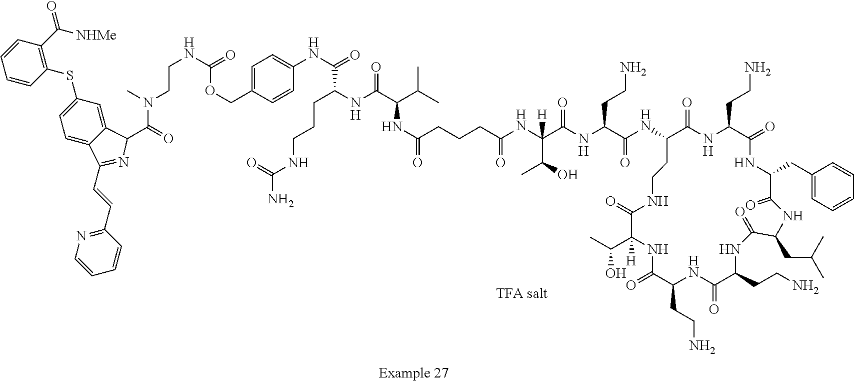 [SMILES: CNC(=O)c1ccccc1Sc1ccc2c(c1)C(C(=O)N(C)CCNC(=O)OCc1ccc(NC(=O)[C@@H](CCCNC(N)=O)NC(=O)[C@H](NC(=O)CCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@H](C)O)C(C)C)cc1)N=C2/C=C/c1ccccn1]

Intermediate 87. Intermediate 29 (50 mg) and tert-butyl (2-(methylamino)ethyl)carbamate (19 mg) were dissolved in DMF (5 mL), added TEA (2.0 eq). The mixture was heated to 70° C. for 5 h, LCMS indicated completion. The reaction was directly purified by HPLC eluting with ACN/Water (0.05% TFA, 0˜100% in 60 min, 18 mL/min) to afford Intermediate 87. MS: 587.0 [M+H⁺].

Intermediate 88. Intermediate 87 was treated with 20% TFA at r.t., and stirred for 30 mins, then volatiles were removed after DCE was added. The crude product was used without further purification. MS: 487.0 [M+H⁺].

Intermediate 90. Intermediate 89 (0.36 g, 0.75 mmol), PnpOC(═O)C₁ (0.18 g, 0.89 mmol) was suspended in THE (40 mL), and DMAP (20 mg), and TEA (0.21 mL) were added. The reaction mixture immediately formed a suspension, which was stored in a −20° C. freezer over weekend. LCMS indicated product formed with about 50% dimer. Volatiles were removed, and the residue was used directly in next step. MS: 645.0 [M+H⁺].

Intermediate 91. Intermediate 90 (14 mg), Intermediate 88 (4 mg) and DMAP (10 mg) were dissolved in DMF (2 mL), to which was added TEA (2.0 eq). The reaction was allowed to proceed at r.t. for 3 h, LC-MS indicated completion. The residue was purified by HPLC eluting with ACN/Water (0.05% TFA). MS: 992.4 [M+H⁺]. The product after lyophilization was treated with 20% TFA at r.t., and stirred for 1 h, then volatiles were removed after added DCE. The crude product was used without further purification. MS: 892.3 [M+H⁺].

Intermediate 93. The mixture of Intermediate 91 (0.10 g), Intermediate 92 (0.11 g), HATU (65 mg), TEA (50 mL) in DMF was stirred at r.t. overnight. The mixture was purified with C18 chromatography eluting with water/ACN containing 0.1% TFA. MS: 1176.1 [M+2H]z.

Compound of Example 27. Intermediate 93 was treated with 20% TFA at r.t., and stirred for 1 h, then volatiles were removed after DCE was added. The crude product was purified with C18 chromatography after the volatiles were removed, eluting with ACN/water containing 0.1% TFA, to afford the Compound of Example 27 (31 mg). MS: 1950.6 [M+H⁺].

### Example 28

### Synthesis of the Compound of Example 28

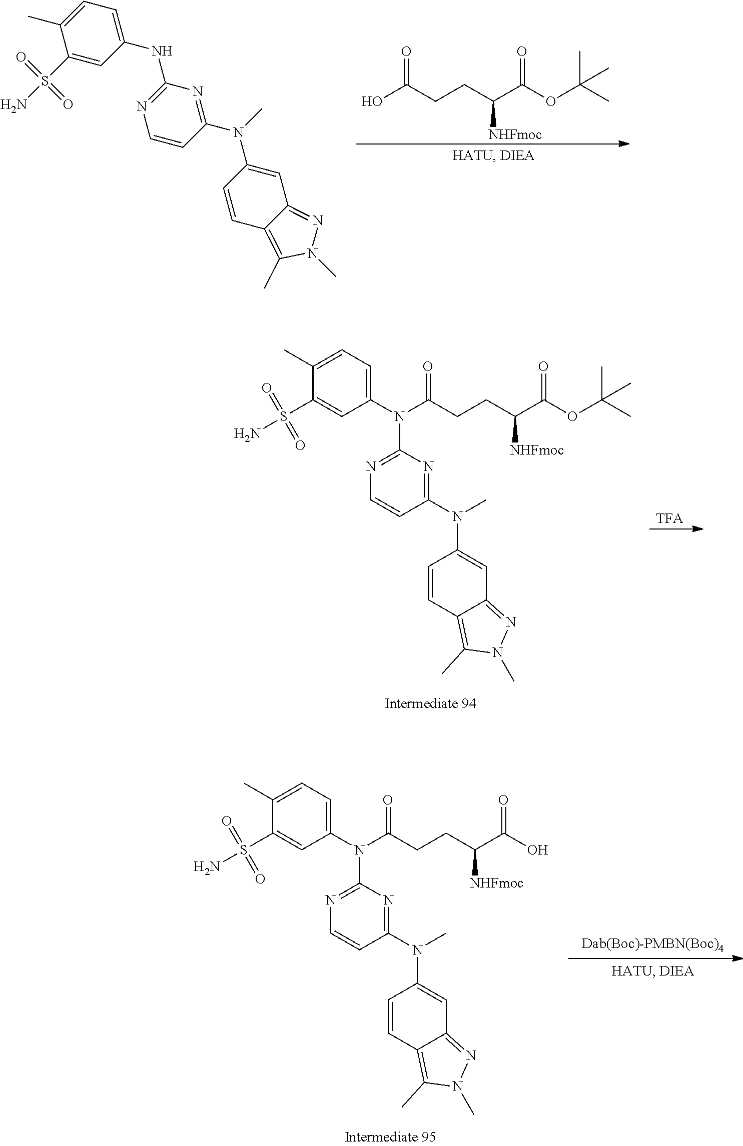

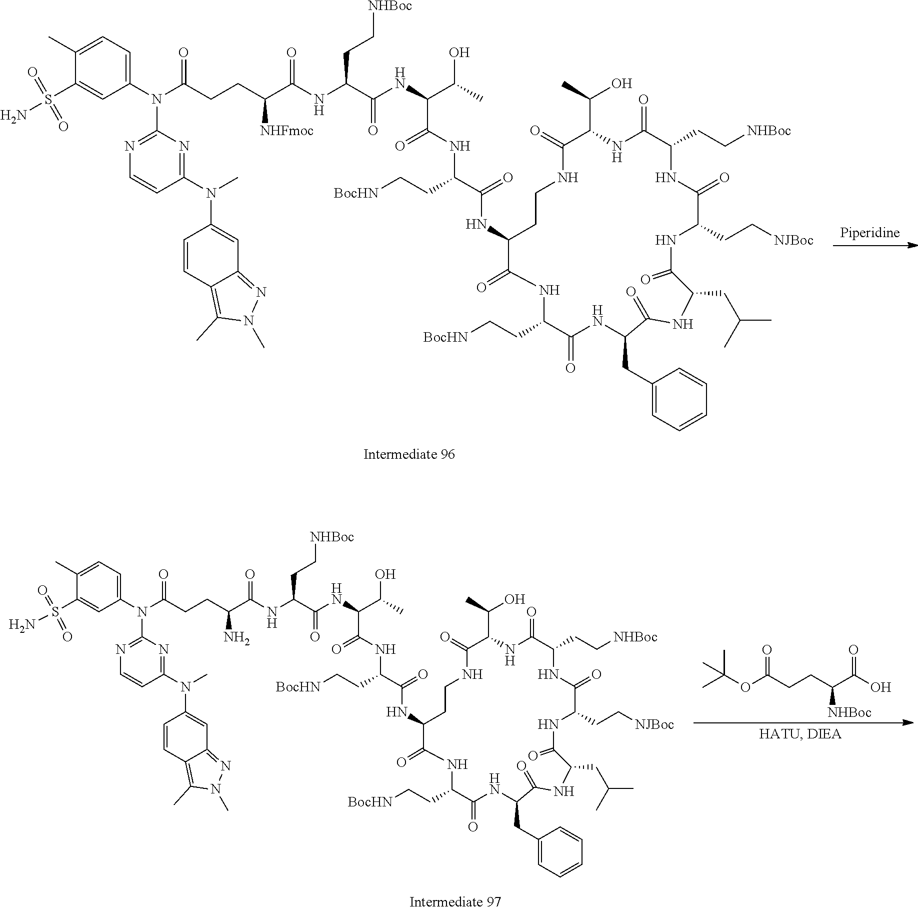

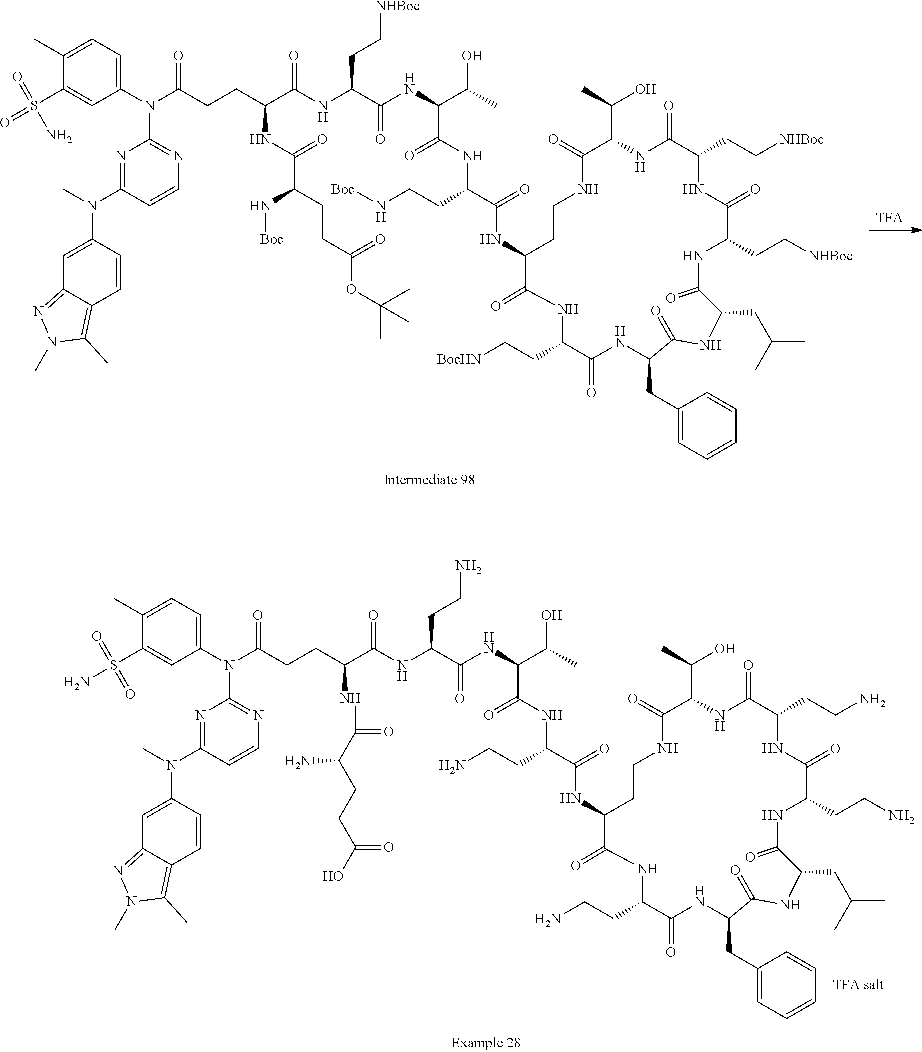

Intermediate 94. A 25 mL flask was charged with 5-((4-((2,3-dimethyl-2H-indazol-6-yl)(methyl)amino)pyrimidin-2-yl)amino)-2-methylbenzenesulfonamide (250.5 mg, 0.5725 mmol), FmocGluOBu-t (243.6 mg, 0.57 mmol), HATU (239.5 mg, 0.63 mmol), DIEA (294 μL, 1.72 mmol) and dry DMF (5 mL) under Ar. Then the reaction mixture was stirred at r.t. overnight, and LC-MS showed the reaction was complete. The reaction mixture was quenched with sat. NH₄Cl aq. sol., extracted three times with EA. The combined EA layers were washed with 5% NaCl aq. sol. twice and brine once, and dried over Na₂SO₄, then evaporated under vacuum and dried under vacuum to give crude product. Purification by HPLC with ACN/Water (0.05% TFA, 0˜100% in 75 min, 18 mL/min) gave Intermediate 94 as a white solid (232.8 mg).

Intermediate 95 A 25 mL flask was charged with Intermediate 94 (232.8 mg, 0.27 mmol), TFA (1 mL) and DCM/DCE (2.5/2.5 mL) under Ar. The reaction mixture was stirred at r.t. overnight. Volatiles were removed under vacuum, and then dried under vacuum to give crude Intermediate 95 as a white solid (296.8 mg).

Intermediate 96. A 50 mL flask was charged with Intermediate 95 (243 mg, 0.22 mmol), Dab(Boc)-PMBN(Boc)₄ (352.7 mg, 0.22 mmol), HATU (94.3 mg, 0.25 mmol), DIEA (116 μL, 0.68 mmol) and dry DMF (5 mL) under Ar. The reaction mixture was stirred at r.t. for 16 h. The reaction mixture was quenched with sat. NH₄Cl aq. sol., extracted with EA for 3 times. The combined EA layers were washed with 5% NaCl aq. sol. twice and brine once, and dried over Na₂SO₄, then evaporated under vacuum and dried under vacuum to give crude product. Purification by HPLC with ACN/Water (0.05% TFA, 0˜100% in 60 min) gave Intermediate 96 as a white solid (413.0 mg).

Intermediate 97. A 10 mL flask was charged with Intermediate 96 (101.4 mg, 0.043 mmol), Piperidine (39.8 μL, 0.43 mmol) and DMF (1 mL) under Ar. The reaction mixture was stirred at r.t. for 0.5 h. The reaction mixture was isolated through C18 chromatography with ACN/Water (0.05% TFA, 5˜100% in 50 min, 15 mL/min) gave Intermediate 97 as a white solid (56.5 mg).

Intermediate 98. A 10 mL flask was charged with Intermediate 97 (56.5 mg, 0.0267 mmol), (S)-5-(tert-butoxy)-2-((tert-butoxycarbonyl)amino)-5-oxopentanoic acid (8.1 mg, 0.0267 mmol), HATU (11.2 mg, 0.0294 mmol), DIEA (13.7 ul, 0.0801 mmol) and dry DMF (1.5 mL) under Ar. The reaction mixture was stirred at r.t. for 4 h. The reaction mixture was quenched with sat. NH₄Cl aq. sol., extracted three times with EA. The combined EA layers were washed with 5% NaCl aq. sol., dried over Na₂SO₄, and then evaporated and dried under vacuum to give crude product. Purification by HPLC with ACN/Water (0.05% TFA, 0˜100% in 60 min, 15 mL/min) gave Intermediate 98 as a white solid (60.9 mg).

Compound of Example 28. A 25 mL flask was charged with Intermediate 98 (60.9 mg, 0.0254 mmol), TFA (0.4 mL) and DCM/DCE (1/1 mL) under Ar. The reaction mixture was stirred at r.t. for 3 h. Volatiles were removed under vacuum, and the residue was purified by HPLC and lyophilized to afford a white solid (36.8 mg). NMR: 8.60 (s, 1H), 7.90 (s, 1H), 7.58 (s, 2H), 7.40-7.34 (m, 3H), 7.24 (d, J=6.8 Hz, 2H), 7.03 (d, J=9.6 Hz, 1H), 6.07 (s, 1H), 4.59˜4.51 (m, 4H), 4.38˜4.35 (m, 2H), 4.32˜4.27 (m, 3H), 4.24-4.18 (m, 4H), 4.13 (s, 3H), 4.09 (t, J=6.4 Hz, 1H), 3.61 (s, 3H), 3.38˜3.11 (m, 1H), 3.20˜3.01 (m, 12H), 2.94˜2.78 (m, 2H), 2.70 (s, 3H), 2.61˜2.44 (m, 7H), 2.29˜1.81 (m, 18H), 1.54˜1.48 (m, 1H), 1.43 (s, 2H), 1.19 (d, J=4.8 Hz, 6H), 0.77 (s, 4H), 0.69 (d, J=6.0 Hz, 3H). MS: 1741.6 [M+H]⁺.

### Example 29

### Synthesis of the Compound of Example 29

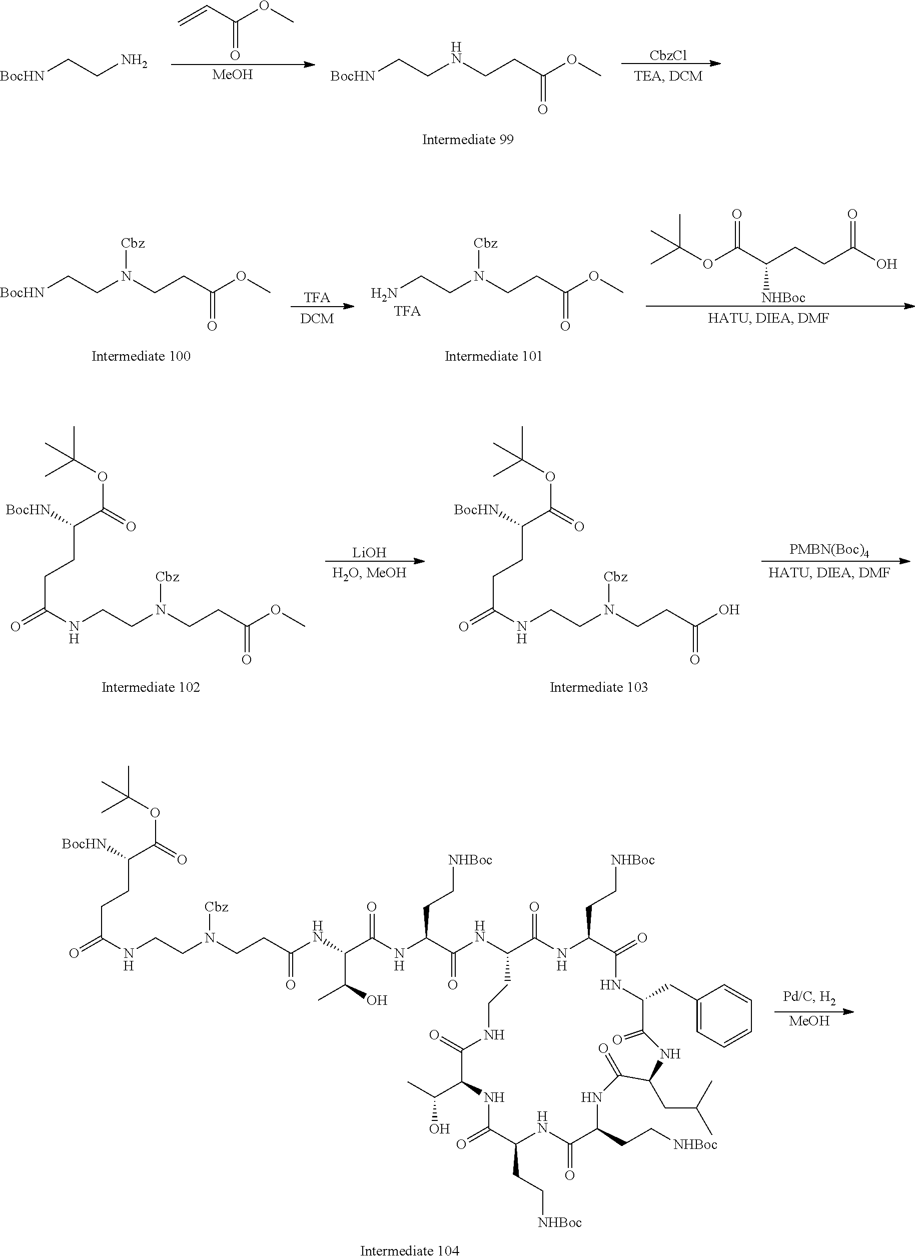

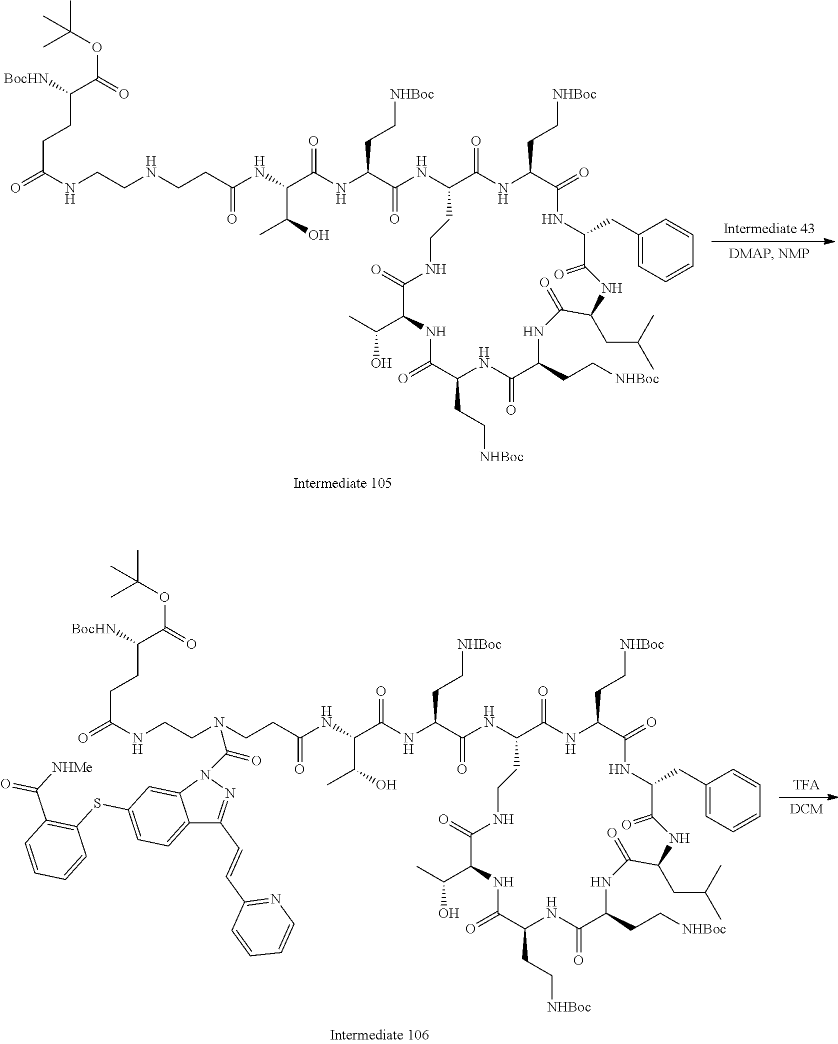

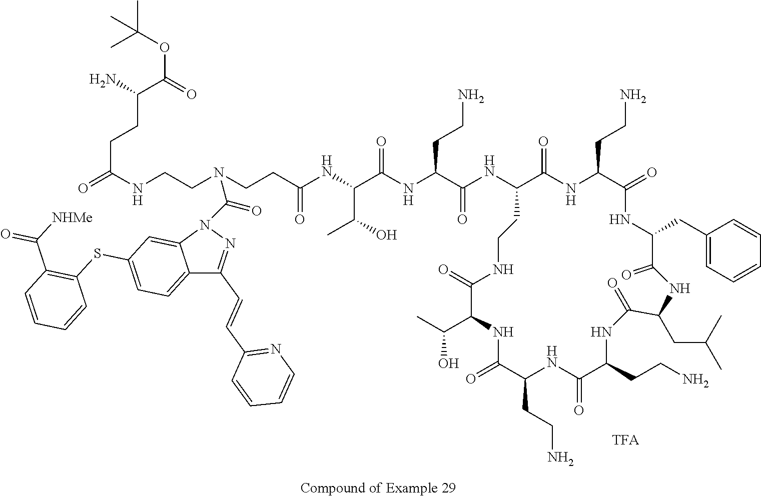 [SMILES: CNC(=O)c1ccccc1Sc1ccc2c(/C=C/c3ccccn3)nn(C(=O)N(CCNC(=O)CC[C@H](N)C(=O)OC(C)(C)C)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 99. The mixture of N-Boc-ethylenediamine (2.4 g, 15 mmol) and methyl acrylate (0.86 g, 10 mmol) in MeOH (4 mL) was stirred at 0-4° C. under Ar for 4 h. Volatiles were evaporated under vacuum, and the residue was purified by silica gel chromatography (gradient 0.1% TEA EA/0.1% TEA PE 0˜100%) to give Intermediate 99 (1.3 g). MS: 247.2 [M+H]⁺.

Intermediate 100. The mixture of Intermediate 99 (1.3 g, 5.3 mmol), CbzCl (1 g, 5.8 mmol) and TEA (1.5 mL, 10.6 mmol) in DCM (10 mL) was stirred at 5° C. under Ar for 4 h. The mixture was removed volatiles, and extracted with EA (50 mL), washed with H₂O (5 mL×2) and brine (5 mL). The EA layer was dried (Na₂SO₄), filtered, and evaporated. The crude material was purified by silica gel chromatography (EA/PE 0˜60%) to give Intermediate 100 (2.0 g). MS: 381.2 [M+H]⁺.

Intermediate 101. Intermediate 100 (2.0 g, 5.26 mmol) in TFA/DCM (2 mL/15 mL) was stirred at r.t. for 1.5 h. Volatiles were removed under vacuum to give Intermediate 101 (2.1 g), used directly at the next step. MS: 281.2 [M+H]⁺.

Intermediate 102. The mixture of Intermediate 101 (2.1 g, 5.26 mmol), (S)-5-(tert-butoxy)-4-((tert-butoxycarbonyl)amino)-5-oxopentanoic acid (1.33 g, 4.38 mmol), HATU (2.33 g, 6.14 mmol) and DIEA (1.55 mL, 8.77 mmol) in DMF (12 mL) was stirred at 40° C. for 4 h. The mixture was cooled to r.t., extracted with EA (100 mL), washed with H₂O (15 mL×2) and brine (15 mL). The EA layer was dried (Na₂SO₄) and solvent evaporated under vacuum. The product was purified by silica gel chromatography (EA/PE 0˜80%) to give Intermediate 102 (2.6 g). MS: 566.1 [M+H]⁺.

Intermediate 103. Intermediate 102 (0.4 g, 0.7 mmol), was added to a solution of LiOH H₂O (45 mg, 1.1 mmol) in MeOH/H₂O (2 mL/1 mL). The reaction was stirred at r.t. for 6.5 h, acidified, and then extracted with EA (50 mL), washed with H₂O (5 mL×2) and brine (5 mL). The EA layer was dried and evaporated. The product was purified by silica gel chromatography (EA/PE 0˜90%) to give Intermediate 103 (0.22 g). MS: 552.0 [M+H]⁺.

Intermediate 104. The mixture of Intermediate 103 (0.22 g, 0.4 mmol), PMBN(Boc)₄; 0.544 g, 0.4 mmol), HATU (0.182 g, 0.48 mmol) and DIEA (0.172 mL, 0.8 mmol) in DMF (6 mL) was stirred at 40° C. for 4 h. The reaction mixture was cooled to r.t., then quenched with water and extracted with EA (100 mL). Organic layer was washed with H₂O (15 mL×2), brine (15 mL), dried (Na₂SO₄) and evaporated under vacuum. The product was purified by C18 column chromatography ACN/H₂O 0˜100%) to give Intermediate 104 (0.49 g). MS: 1896.5 [M+H]⁺.

Intermediate 105. The mixture of Intermediate 104 (0.49 g, 0.28 mmol) and 10% Pd/C (0.1 g) in MeOH (10 mL) was stirred at r.t. under H₂ for 4.5 h, filtered, evaporated, and dried to afford Intermediate 105 (0.385 g). MS: 1762.8 [M+H]⁺.

Intermediate 106. The mixture of Intermediate 105 (0.35 g, 0.2 mmol), Intermediate 43 (0.175 g, 0.35 mmol) and DMAP (0.049 g, 0.4 mmol) in NMP (5 mL) was stirred at 50° C. under Ar for 5 h. The mixture was cooled to r.t., extracted with EA (50 mL), washed with H₂O (5 mL×2) and brine (5 mL). The EA layer was dried (Na₂SO₄) and evaporated. The product was purified by C18 column chromatography (ACN/H₂O 0˜90%) to give Intermediate 106 (0.208 g).

Compound of Example 29. Intermediate 106 (0.27 g, 0.124 mmol) in TFA (1 mL) and DCM (6 mL) was stirred at r.t. for about 1 h. Volatiles were removed under vacuum and the residue was purified by C18 column chromatography (ACN/H₂O 0˜40%) to afford the compound of Example 29 (125 mg) as a TFA salt. MS: 1618.9 [M+H]⁺. NMR: 8.44 (d, J 8.2 Hz, 1H), 8.25 (d, J 8.0 Hz, 1H), 7.95 (s, 2H), 7.82 (t, J 6.7 Hz, 1H), 7.66 (s, 2H), 7.49 (s, 1H), 7.47-7.40 (m, 3H), 7.31-7.20 (m, 5H), 7.14 (d, J 7.6 Hz, 2H), 4.48 (t, J 8.1 Hz, 1H), 4.36 (d, J 7.0 Hz, 2H), 4.23-4.16 (m, 3H), 4.14-4.00 (m, 7H), 3.87 (s, 2H), 3.75 (s, 3H), 3.37 (s, 1H), 3.20 (d, J 13.0 Hz, 1H), 3.00 (d, J 43.2 Hz, 12H), 2.72 (d, J 53.7 Hz, 4H), 2.62 (s, 4H), 2.24 (t, J 7.6 Hz, 2H), 2.13 (s, 7H), 2.02-1.65 (m, 10H), 1.08-1.04 (m, 3H), 0.97 (s, 3H), 0.73 (s, 1H), 0.67 (d, J 6.5 Hz, 3H), 0.60 (d, J 6.4 Hz, 3H).

Optionally, the TFA salt of the compound of Example 29 is converted into an HCl salt, a H₂SO₄ salt, a citric acid salt, a lactic acid salt, a mandelic acid salt, or another pharmaceutically acceptable salt. As generally applicable, this can be accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 30

### Synthesis of the Compound of Example 30

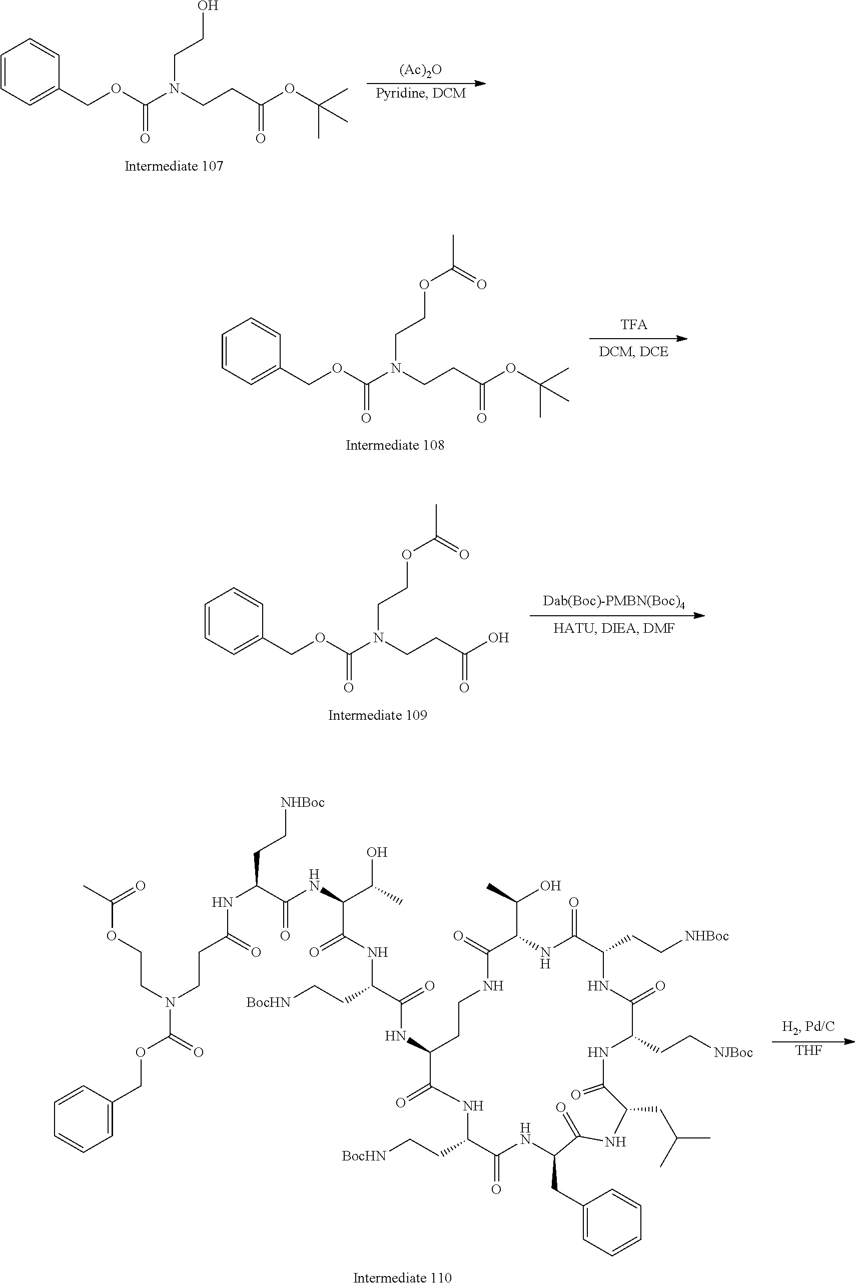

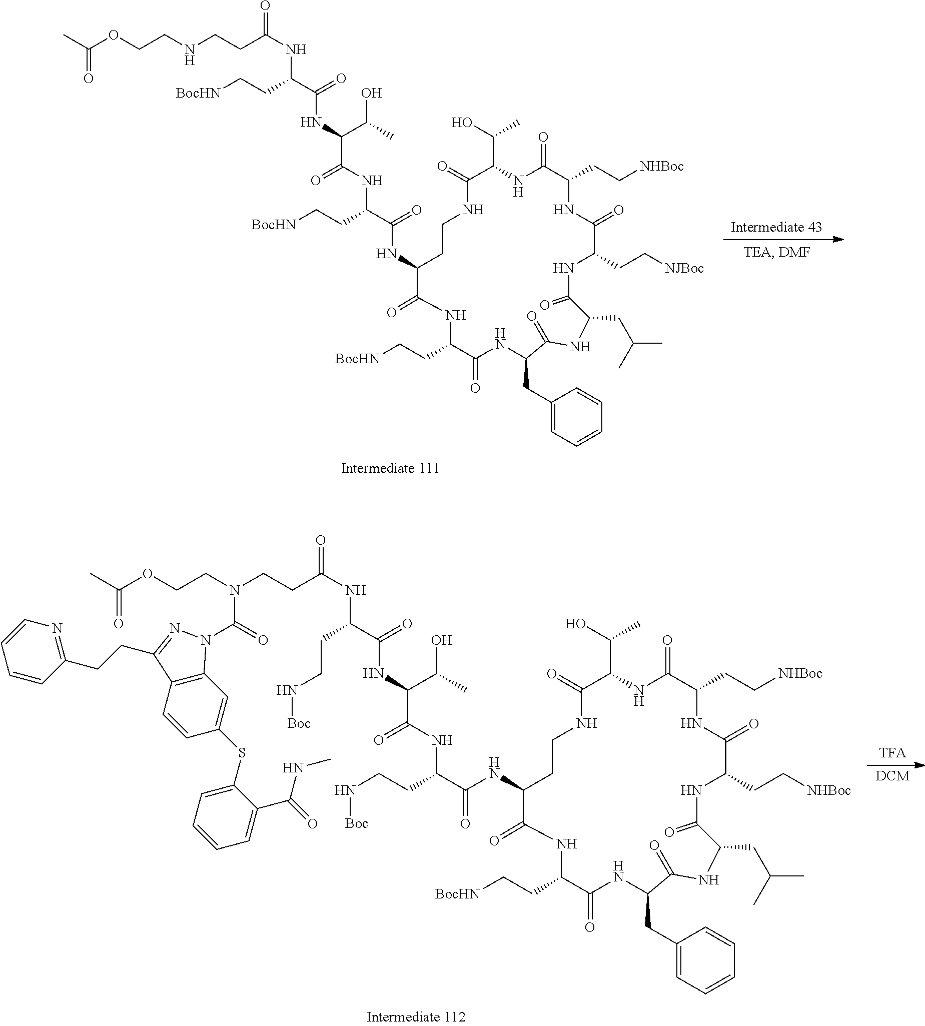

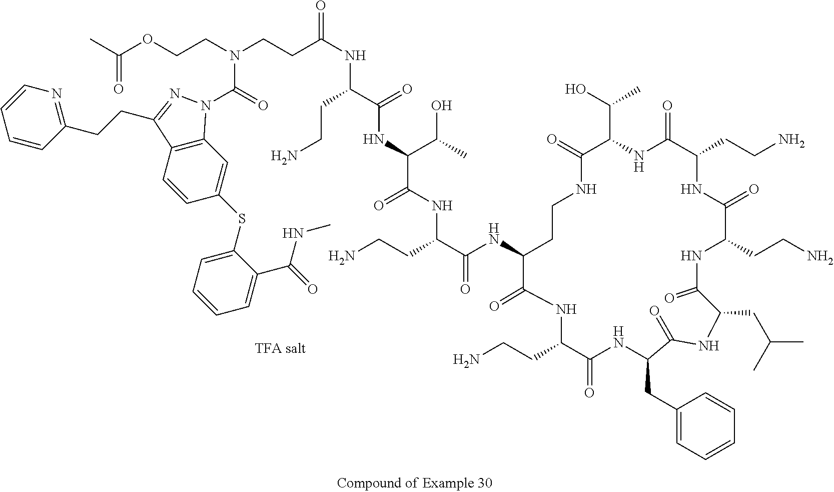 [SMILES: CNC(=O)c1ccccc1Sc1ccc2c(CCc3ccccn3)nn(C(=O)N(CCOC(C)=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 108. A 50 mL flask was charged with Intermediate 107 (5.12 g, 15.83 mmol), Ac₂O (1.65 mL, 17.42 mmol), pyridine (1.91 mL, 23.75 mmol) and dry DCM (20 mL) under Ar. The reaction mixture was stirred at r.t. o.n. Additional Ac₂O (7.92 mmol) and pyridine (11.87 mmol) were added. After stirring at r.t. for another 3 h, the reaction mixture was quenched with sat. NH₄Cl aq. sol., the organic layer was separated, the aqueous was extracted with DCM twice. The combined DCM layer was washed with 0.1% aq. HCl twice, brine once, dried (Na₂SO₄), filtered, evaporated under vacuum and dried under high vacuum. The crude was purified through a silica gel chromatography with EA/PE (1:1 to 2:1 in 40 min, 18 mL/min) to give Intermediate 108 as a colorless oil (5.28 g). MS: 365.91 [M+H]⁺, 388.10 [M+Na]⁺.

Intermediate 109 The mixture of Intermediate 108 (1.695 g, 4.64 mmol), TFA (1 mL) and DCM/DCE (2.5/2.5 mL) under Ar. was stirred at r.t. o.n. Volatiles were removed under reduced pressure, then dried under high vacuum to afford the Intermediate 109 as colorless oil (1.86 g). MS: 310.04 [M+H]⁺.

Intermediate 110. A 50 mL flask was charged with Intermediate 109 (177.2 mg, 0.44 mmol), H-Dab(Boc)-Thr-Dab(Boc)-cyclo[Dab(Boc)-Dab(Boc)-D-Phe-Leu-Dab(Boc)-Dab(Boc)-Thr] (same as Dab(Boc)PMBN(Boc)₄; 625.5 mg, 0.40 mmol), HATU (167.3 mg, 0.44 mmol), DIEA (205 ul, 1.20 mmol) and dry DMF (10 mL) under Ar. The reaction mixture was stirred at r.t. for 2-3 h, quenched with sat. NH₄Cl aq. sol., and extracted three times with EA. The combined EA layer was washed with 5% NaCl aq. sol. Twice and brine once, and dried over anh. Na₂SO₄, then filtered, concentrated under vacuum and dried under high vacuum. The crude was purified through C-18 column with 0.05% TFA in ACN/H₂O (10˜100%) to give Intermediate 110 as a white solid (868.5 mg). MS: 1854.52 [M+H]⁺.

Intermediate 111. The suspension of Intermediate 110 (217.1 mg, 0.117 mmol), 10% Pd/C (60% water, 20 mg) in THF (10 mL) under H₂ was degassed, and stirred under H₂ at r.t. o.n. The reaction mixture was filtered through Celite, washed with THF, concentrated under vacuum, and then dried under high vacuum to give crude Intermediate 111 as a white solid (252.7 mg). MS: 1720.71 [M+H]⁺.

Intermediate 112. The reaction mixture of Intermediate 43 (crude, 99.7 mg, 0.117 mmol), Intermediate 111 (crude, 252.7 mg, 0.117 mmol), TEA (32.5 μL, 0.234 mmol) and dry DMF (2.5 mL) under Ar was stirred at r.t. for 6 h, and then purified through C-18 column with ACN/Water (0.05% TFA, 50˜100% in 40 min, 15 mL/min) to afford Intermediate 112 as a white solid (90.9 mg).

Compound of Example 30. A 10 mL flask was charged with Intermediate 112 (90.9 mg, 0.0426 mmol), TFA (0.2 mL) and DCM/DCE (1/1 mL) under Ar. The reaction mixture was stirred at r.t. for 3 h. Volatiles were removed under vacuum, and the crude product was purified through C-18 column eluting with 0.05% TFA in ACN/H₂O (5˜100%) and lyophilized to afford the Example of Compound 30 as TFA salt: white solid (36.0 mg). MS: 1632.68 [M+H]⁺. NMR: 8.54 (d, J 5.6 Hz, 1H), 8.38 (t, J 6.8 Hz, 1H), 8.17 (d, J 8.0 Hz, 1H), 7.87 (d, J 9.2 Hz, 1H), 7.83 (d, J 15.2 Hz, 1H), 7.75 (t, J 6.8 Hz, 1H), 7.57 (d, J 14.8 Hz, 2H), 7.44-7.38 (m, 5H), 7.25-7.17 (m, 5H), 7.10 (d, J 7.2 Hz, 2H), 4.42 (t, J 8.4 Hz, 1H), 4.37˜4.33 (m, 3H), 4.19 (d, J 4.4 Hz, 3H), 4.15˜4.02 (m, 8H), 3.82˜3.78 (m, 3H), 3.21˜3.14 (m, 1H), 3.02˜2.90 (m, 13H), 2.78-2.70 (m, 1H), 2.66-2.61 (m, 3H), 2.55 (s, 3H), 2.13˜2.01 (m, 7H), 1.97-1.86 (m, 4H), 1.80-1.71 (m, 6H), 1.38-1.22 (m, 2H), 1.03 (dd, J 10.8, 4.4 Hz, 7H), 0.62 (s, 4H), 0.54 (d, J 4.8 Hz, 3H).

Optionally, the TFA salt of the compound of Example 30 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 31

### Synthesis of the Compound of Example 31

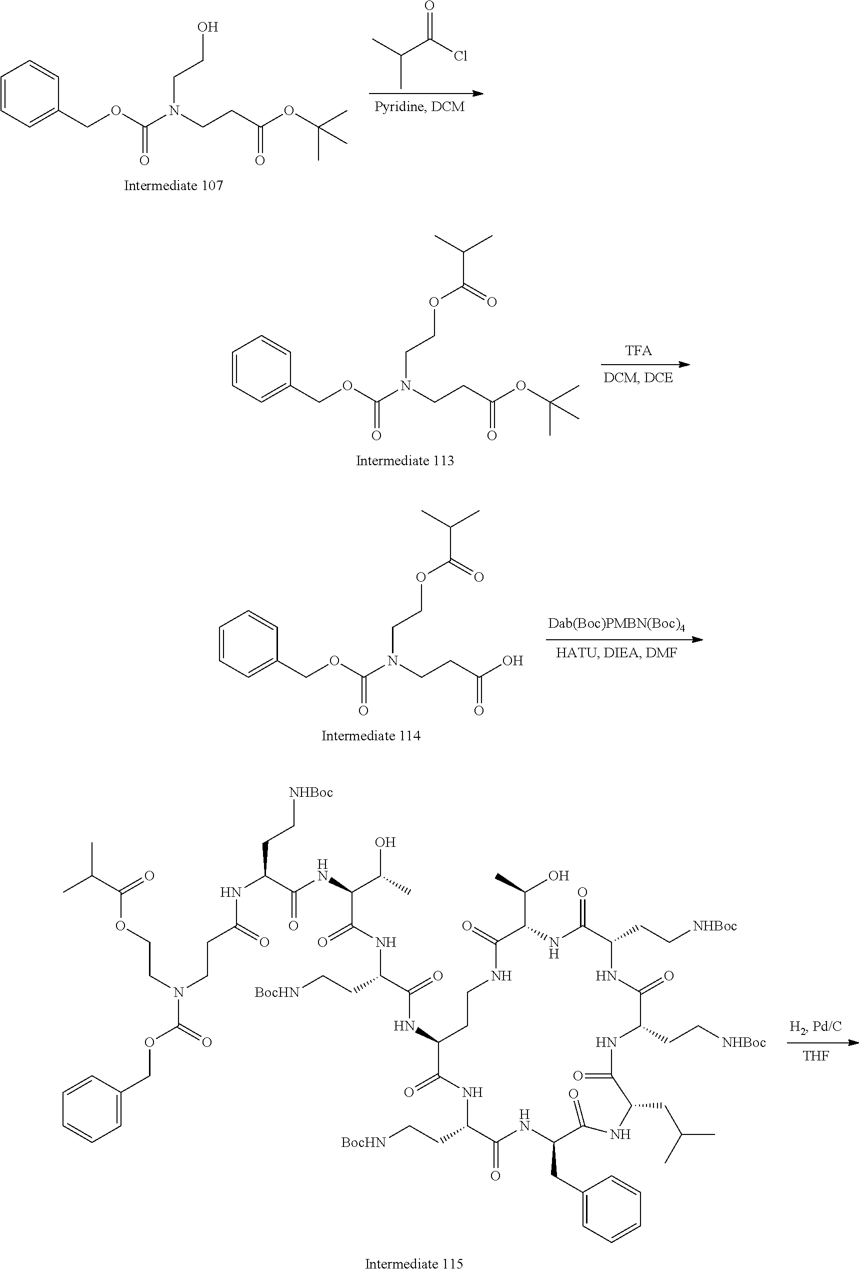

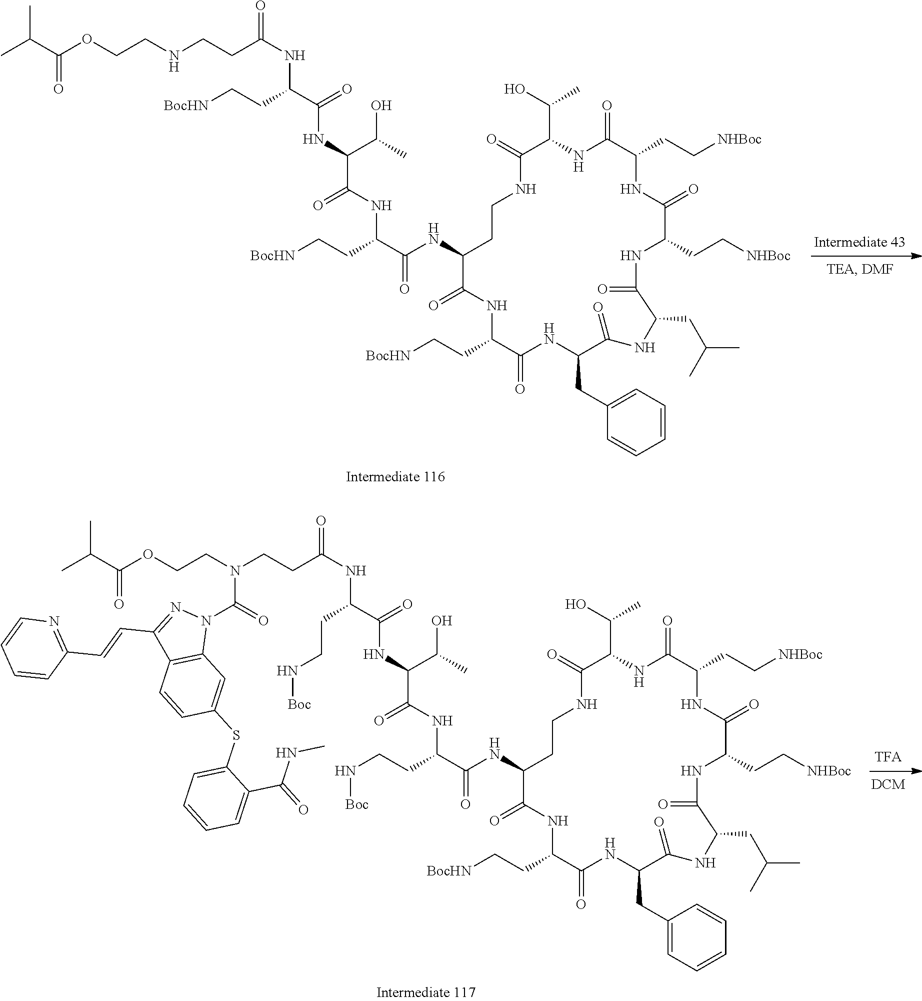

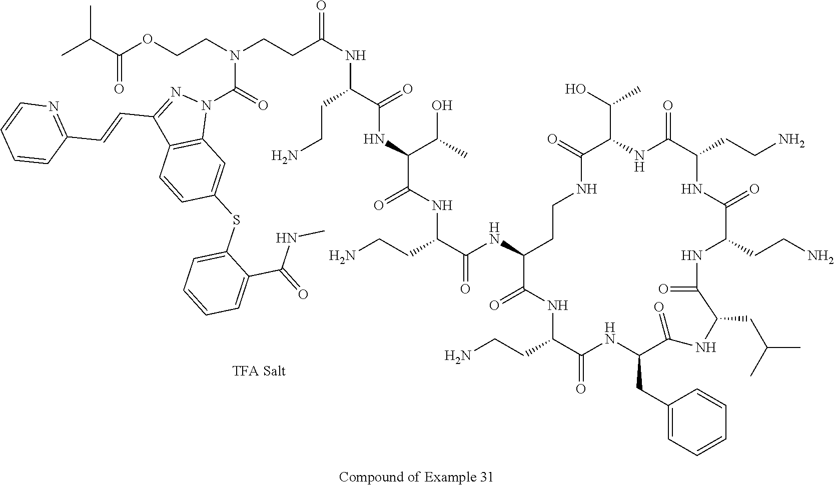 [SMILES: CNC(=O)c1ccccc1Sc1ccc2c(/C=C/c3ccccn3)nn(C(=O)N(CCOC(=O)C(C)C)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 113. The mixture of Intermediate 107 (2 g, 6.2 mmol), isobutyryl chloride (0.72 mL, 6.8 mmol) and Py (0.75 mL, 9.3 mmol) in DCM (20 mL) was stirred at r.t. for 4 h. The mixture was extracted with DCM (100 mL), washed with H₂O (15 mL×2) and brine (15 mL). The DCM layer was dried and evaporated. The product was purified by silica gel column (EA/PE=0˜50%) to give Intermediate 113 (2.1 g). MS: 393.9 [M+H]⁺.

Intermediate 117. Intermediate 117 was made analogously to the procedures used for the preparation of Intermediate 112 (in the synthesis of the compound of Example 30), except starting from Intermediate 113 instead of Intermediate 108 used in the synthesis of the compound of Example 30.

Compound of Example 31. The mixture of Intermediate 117 (0.153 g, 0.07 mmol) in TFA/DCM (1 mL/3 mL) was stirred at r.t. for 1.5 h. The volatiles were removed, and the residue was purified by C18 column chromatography (ACN/H₂O=0˜60%) to afford the compound of Example 31 as TFA salt (125 mg). MS: 1660.8 [M+H]⁺. NMR: δ 8.56 (d, J 5.5 Hz, 1H), 8.34 (s, 1H), 8.14 (s, 1H), 7.73 (s, 1H), 7.67-7.57 (m, 2H), 7.41 (s, 3H), 7.23 (dd, J 12.1, 7.0 Hz, 4H), 7.12 (d, J 7.3 Hz, 2H), 4.36 (d, J 6.9 Hz, 3H), 4.24-4.01 (m, 11H), 3.92 (s, 2H), 3.82 (s, 2H), 3.18 (s, 1H), 3.07-2.88 (m, 12H), 2.65 (s, 3H), 2.59 (s, 3H), 2.09 (s, 7H), 1.80 (s, 3H), 1.04 (dd, J 9.9, 6.4 Hz, 7H), 0.80 (d, J 6.7 Hz, 6H), 0.65 (s, 3H), 0.57 (d, J 6.0 Hz, 3H).

Optionally, the TFA salt of the compound of Example 31 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 32

### Synthesis of the Compound of Example 32

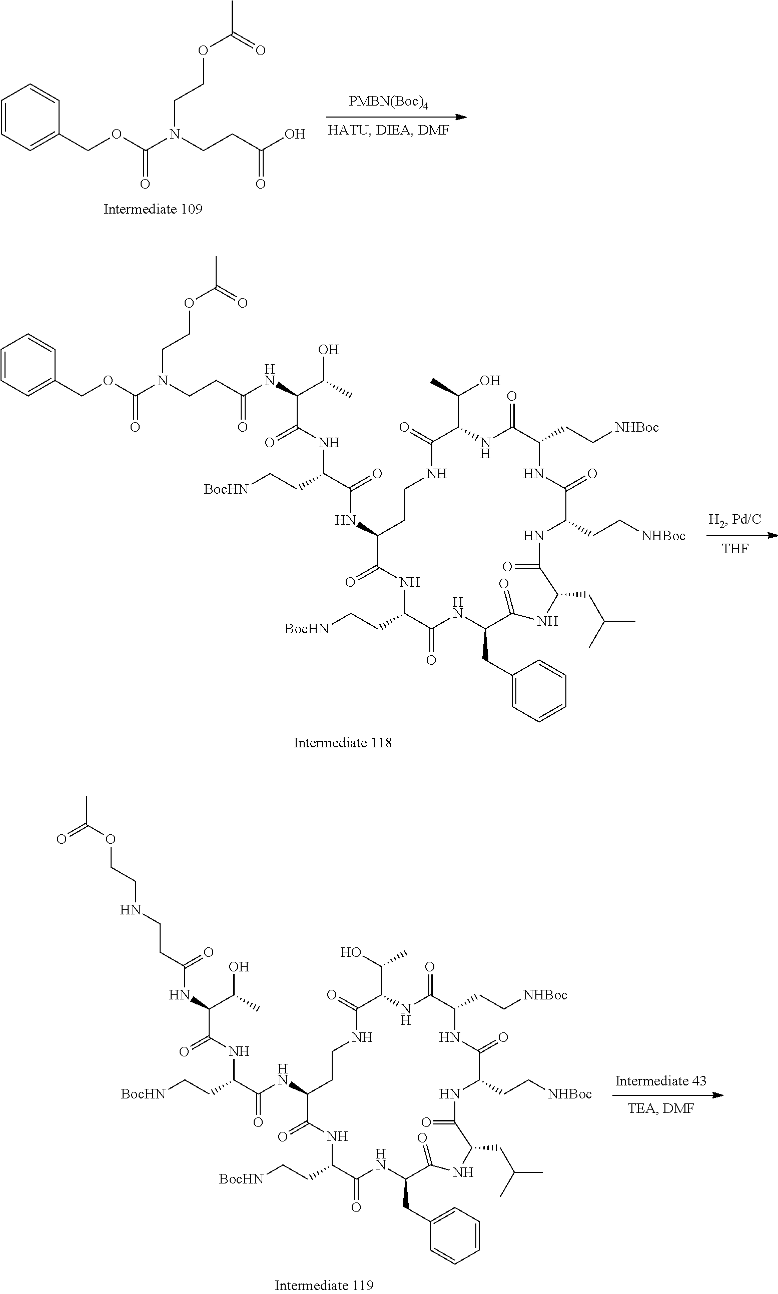

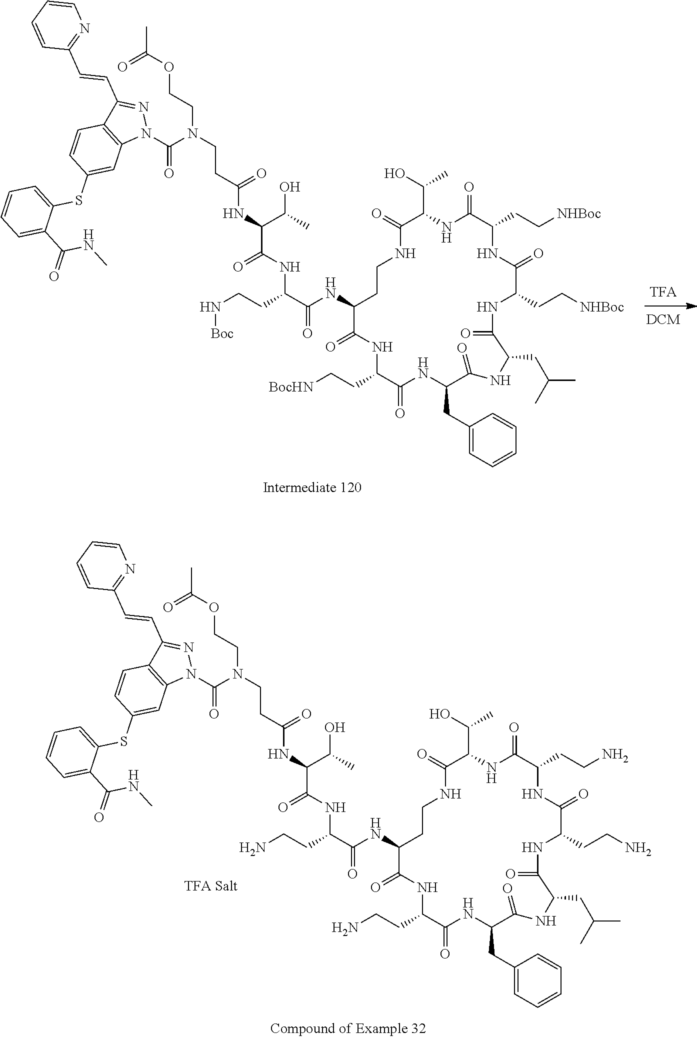

Compound of Example 32. The compound of Example 32 was made according to a procedures described for the synthesis of the compound of Example 30, except that Intermediate 109 was coupled with PMBN(Boc)₄ instead of Dab(Boc)PMBN(Boc)₄ (used in the synthesis of the compound of Example 30) to prepare Intermediate 118. The latter was converted to Intermediate 119, and thereafter to Intermediate 120 just as described above for respective preparations of Intermediates 111 and 112 (used in the preparation of the compound of Example 30). The compound of Example 32 was purified by C18 chromatography and isolated as TFA salt: a white solid (66.2 mg). MS: 1532.64 [M+H]⁺. NMR: 8.55 (d, J 5.8 Hz, 1H), 8.38 (t, J 7.9 Hz, 1H), 8.18 (d, J 8.4 Hz, 1H), 7.88 (d, J 9.3 Hz, 1H), 7.82 (s, 1H), 7.76 (t, J 6.8 Hz, 1H), 7.58 (d, J 15.5 Hz, 2H), 7.43 (s, 1H), 7.41-7.34 (m, 3H), 7.21 (dt, J 17.6, 8.2 Hz, 5H), 7.10 (d, J 7.4 Hz, 2H), 4.42 (t, J 8.4 Hz, 1H), 4.35 (q, J 4.5 Hz, 3H), 4.24-3.98 (m, 12H), 3.80 (d, J 15.4 Hz, 4H), 3.24-3.12 (m, 1H), 2.96 (td, J 19.1, 16.0, 9.8 Hz, 13H), 2.74 (s, 1H), 2.63 (s, 3H), 2.56 (s, 4H), 2.18-1.98 (m, 7H), 1.93 (d, J 11.6 Hz, 4H), 1.75 (d, J 32.0 Hz, 6H), 1.34 (d, J 8.5 Hz, 1H), 1.26 (t, J 12.3 Hz, 1H), 1.03 (dd, J 11.2, 6.3 Hz, 7H), 0.62 (d, J 5.1 Hz, 4H), 0.54 (d, J 5.6 Hz, 4H).

Optionally, the TFA salt of the compound of Example 32 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product can be optionally recrystallized, for example from an alcohol-containing media, such as EtOH-EtOAc, or isopropanol-water, or similar solvent system.

### Example 33

### Synthesis of the Compound of Example 33

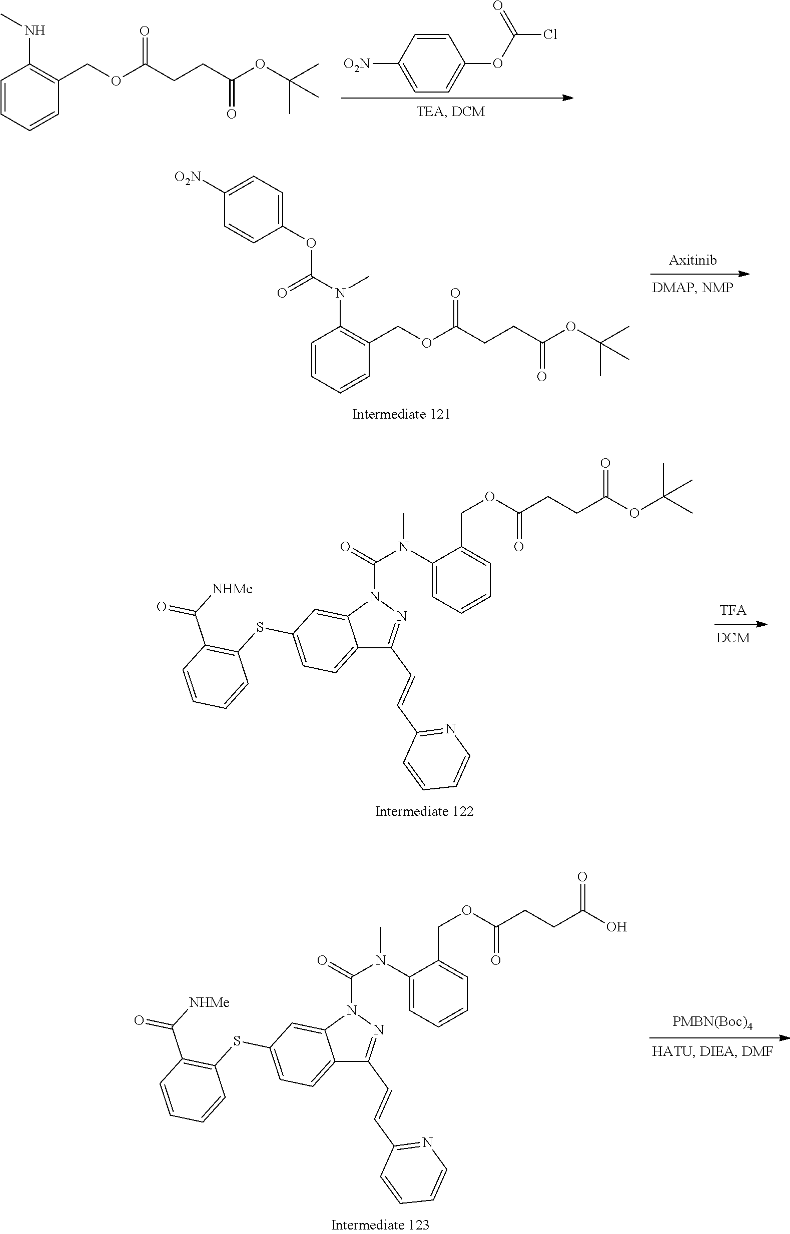

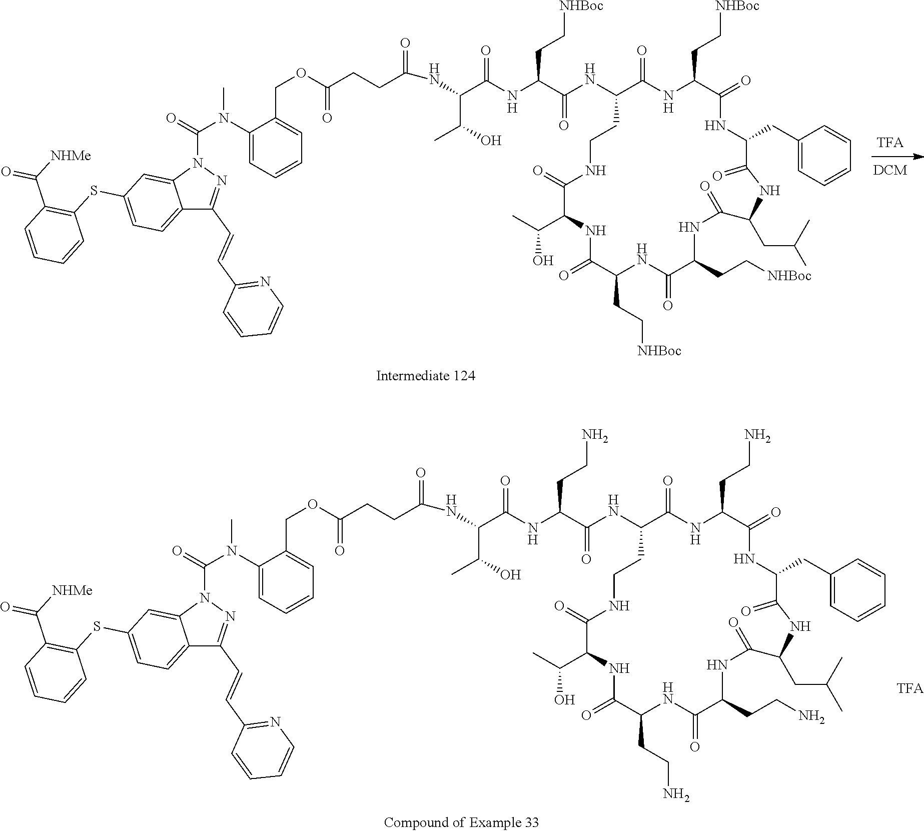

Intermediate 121. The mixture of tert-butyl 2-(methylamino)benzyl succinate (293 mg. 1 mmol). 4-nitrophenyl chloroformate (same as PnpOC(═O)Cl: 211 mg. 1.05 mmol) and TEA (0.277 mL, 2 mmol) in DCM (5 mL) was stirred at r.t. for 4.5 h. The mixture was extracted with DCM (20 mL), washed with brine (3 mL×2). The DCM layer was dried and evaporated, and the product purified by silica gel column to give Intermediate 121 (465 mg).

Intermediate 122 The mixture of Intermediate 121 (465 mg, 1 mmol), (E)-N-methyl-2-((3-(2-(pyridin-2-yl)vinyl)-1H-indazol-6-yl)thio)benzamide (same as axitinib; 322 mg, 0.83 mmol) and DMAP (152 mg, 1.25 mmol) in NMP (5 mL) was stirred at 65° C. for 18 h. The reaction was cooled to r.t., extracted with EA (50 mL), washed with brine (5 mL×3). The EA layer was dried and evaporated, and the product purified by C18 column chromatography ACN/H₂O=0˜90%) to give Intermediate 122 (380 mg). MS: 706.1 [M+H]⁺.

Intermediate 123 The solution of Intermediate 122 (105 mg, 0.15 mmol) in 90% aq. HCOOH (2.2 mL) was stirred under Ar at 35° C. for 1.5 h. Volatiles were removed under vacuum, and the residue was purified by C18 column chromatography ACN/H₂O 0˜50%) to give Intermediate 123 (100 mg). MS: 650.2 [M+H]⁺.

Intermediate 124 The mixture of Intermediate 123 (81 mg, 0.12 mmol), PMBN(Boc)₄ (187 mg, 0.14 mmol), HATU (57 mg, 0.15 mmol) and DIEA (0.044 mL, 0.25 mmol) in DMF (3 mL) was stirred under Ar at r.t. for 4 h. The mixture was extracted with EA (40 mL), washed with H₂O (5 mL×2) and brine (5 mL). The EA layer was dried and evaporated. The product was purified by C18 column chromatography ACN/H2O=0˜100%) to give Intermediate 124 (112 mg). MS: 1994.6 [M+H]⁺.

Compound of Example 33. The mixture of Intermediate 124 (112 mg, 0.056 mmol) in TFA/DCM (0.3 mL/3 mL) was stirred at r.t. for 0.5 h. Volatiles were removed, and the residue was purified by C18 column chromatography ACN/H₂O 0˜60%) to afford the compound of Example 33 (81 mg) as TFA salt. MS: 1594.6 [M+H]⁺. NMR: 8.47 (d, J 5.7 Hz, 1H), 8.23 (t, J 8.0 Hz, 1H), 8.00 (d, J 8.2 Hz, 1H), 7.65 (d, J 7.8 Hz, 2H), 7.57 (t, J 13.7 Hz, 2H), 7.43 (dd, J 18.0, 7.8 Hz, 4H), 7.37-7.13 (m, 10H), 7.06 (d, J 6.9 Hz, 4H), 5.41-5.22 (m, 2H), 4.43 (dt, J 8.5, 4.6 Hz, 1H), 4.31 (d, J 6.2 Hz, 2H), 4.21-3.95 (m, 10H), 3.20 (s, 2H), 3.06 (s, 2H), 2.93 (d, J 29.5 Hz, 11H), 2.66 (s, 4H), 2.54 (d, J 3.9 Hz, 3H), 2.06 (d, J 35.9 Hz, 8H), 1.75 (d, J 44.3 Hz, 5H), 1.28 (s, 2H), 1.08-0.99 (m, 6H), 0.96 (d, J 6.1 Hz, 1H), 0.62 (d, J 3.7 Hz, 3H), 0.56-0.48 (m, 3H).

### Example 34

### Synthesis of the Compound of Example 34

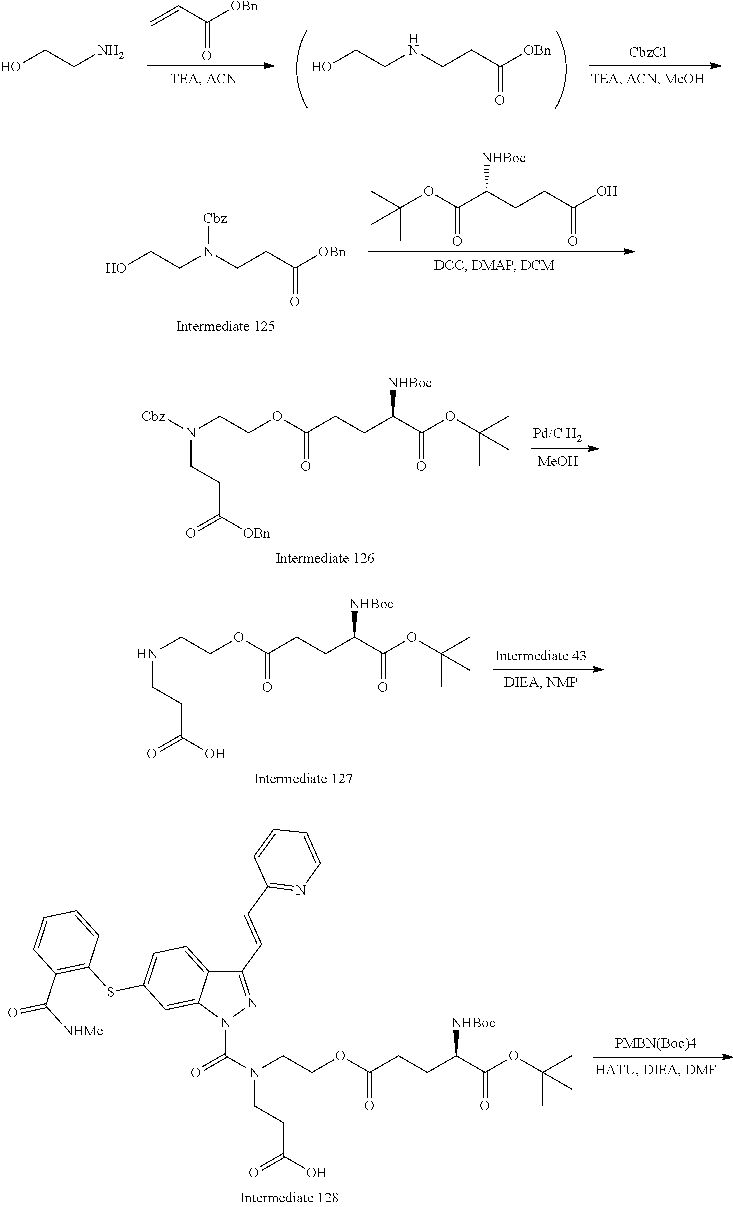

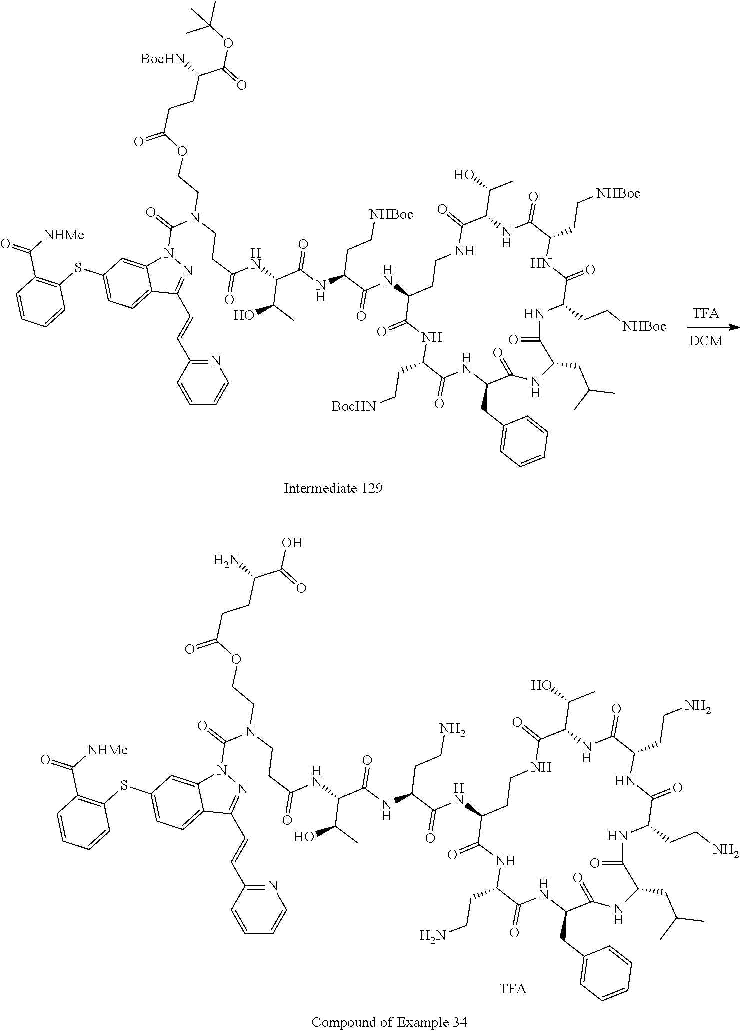

Intermediate 125. The mixture of hydroxylethylamine (1.34 g, 22 mmol), benzyl acrylate (3.24 g, 20 mmol) and TEA (2.02 g, 20 mmol) in ACN (25 mL) was stirred at 50° C. under Ar for 4 h and cooled to r.t. CbzCl (4.1 g, 24 mmol) and TEA (4.4 g, 44 mmol) in ACN/MeOH (25 mL/15 mL) were added. The reaction was stirred at 25° C. under Ar for 3 h. Volatiles were removed. The mixture was extracted with EA (80 mL), organic layer was washed with H₂O (10 mL×2) and brine (10 mL). The EA layer was dried (Na₂SO₄) and evaporated under vacuum. The product was purified by silica gel chromatography (gradient EA/PE=0˜80%) to give Intermediate 125 (3.5 g). MS: 358.1 [M+H]⁺.

Intermediate 126. The mixture of Intermediate 125 (3.3 g, 9.2 mmol), (R)-5-(ter.t-butoxy)-4-((tert-butoxycarbonyl)amino)-5-oxopentanoic acid (2.8 g, 9.2 mmol), DCC (2.3 g, 11.1 mmol) and DMAP (1.4 g, 11.1 mmol) in DCM (50 mL) was stirred at 25° C. for 14 h. and then filtered. The filtrate was washed with brine (25 mL). The DCM layer was dried and evaporated. The product was purified by silica gel chromatography (EA/PE 0˜80%) to give Intermediate 126 (5.1 g). MS: 665.3 [M+Na]⁺.

Intermediate 127. The suspension of Intermediate 126 (2 g, 3.1 mmol) and Pd/C (0.5 g, 56% H₂O) in MeOH (20 mL) was degassed with H₂, then stirred under H₂ at r.t. for 3 h. The mixture was filtered. The filtrate was evaporated to give Intermediate 127 (1.3 g). MS: 419.2 [M+H]⁺.

Intermediate 128. Intermediate 43 (0.22 g, 0.4 mmol), Intermediate 127 (0.168 g, 0.4 mmol) and DIEA (0.142 mL, 0.8 mmol) were dissolved in NMP (3 mL), and the mixture was stirred at 25° C. under Ar for 4 h. The mixture was diluted with water, extracted with EA (50 mL), washed with H₂O (10 mL×2) and brine (10 mL). The EA layer was dried (Na₂SO₄) and evaporated. The product was purified by C18 column chromatography ACN/H₂O 0˜80%) to give Intermediate 128 (0.215 g). MS: 831.2 [M+H]⁺.

Intermediate 129. The mixture of Intermediate 128 (0.208 g, 0.25 mmol), PMBN(Boc)₄ (0.341 g, 0.25 mmol), HATU (0.114 g, 0.3 mmol) and DIEA (0.089 mL, 0.5 mmol) in DMF (5 mL) was stirred at 25° C. under Ar for 3.5 h. The mixture was diluted with water and extracted with EA (40 mL). The organic layer was washed with H₂O (5 mL×2) and brine (5 mL), dried (Na₂SO₄) and evaporated under vacuum. The product was purified by C18 column chromatography ACN/H₂O 0˜90%) to give Intermediate 129 (0.160 g).

Compound of Example 34. Intermediate 129 (0.235 g, 0.108 mmol) in TFA/DCM (1 mL/3 mL) was stirred at r.t. for 4 h. Volatiles were removed under vacuum and the residue was purified on C18 column (ACN/H₂O 0˜40%) to afford the compound of Example 34 as TFA salt (955 mg). MS: 1619.7 [M+H]⁺. ¹NMR: 8.45 (td, J 8.1, 1.6 Hz, 1H), 8.22 (d, J 8.4 Hz, 1H), 7.87-7.79 (m, 2H), 7.57 (d, J 2.9 Hz, 1H), 7.42-7.34 (m, 4H), 7.28-7.07 (m, 7H), 4.44 (s, 1H), 4.39-4.24 (m, 4H), 4.21-4.01 (m, 9H), 3.79 (d, J 37.1 Hz, 5H), 3.20 (dd, J 14.2, 7.6 Hz, 1H), 2.96 (q, J 8.3, 7.2 Hz, 10H), 2.71 (dt, J 43.7, 7.9 Hz, 5H), 2.60 (d, J 3.3 Hz, 3H), 2.35 (s, 1H), 2.18-2.04 (m, 5H), 1.96 (s, 5H), 1.78 (d, J 39.0 Hz, 4H), 1.42-1.24 (m, 2H), 1.09-0.93 (m, 6H), 0.64 (d, J 5.0 Hz, 3H), 0.56 (d, J 5.8 Hz, 3H).

Optionally, the TFA salt of the compound of Example 34 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product can be optionally recrystallized, for example from an alcohol-containing media, such as EtOH-EtOAc, or isopropanol-water, or similar solvent system.

### Example 35

### Synthesis of the Compound of Example 35

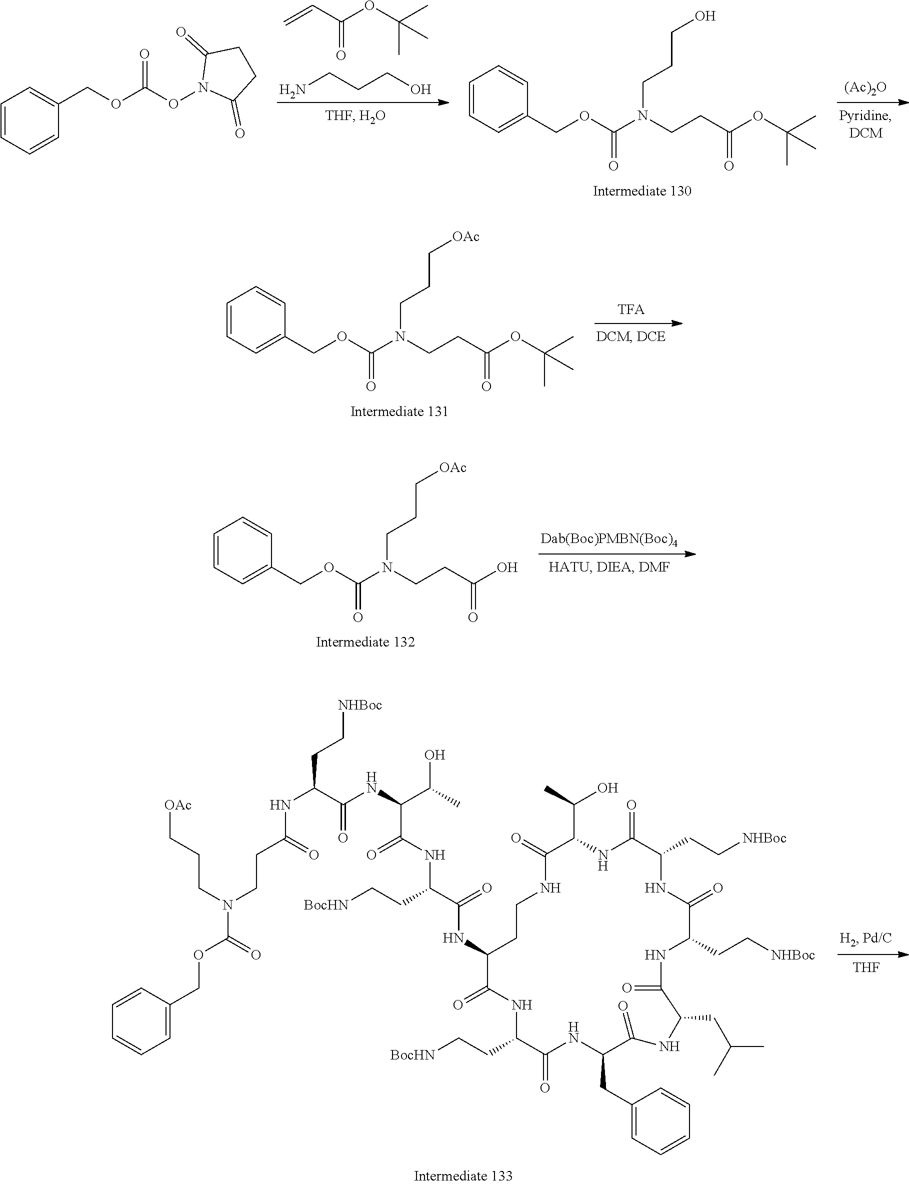

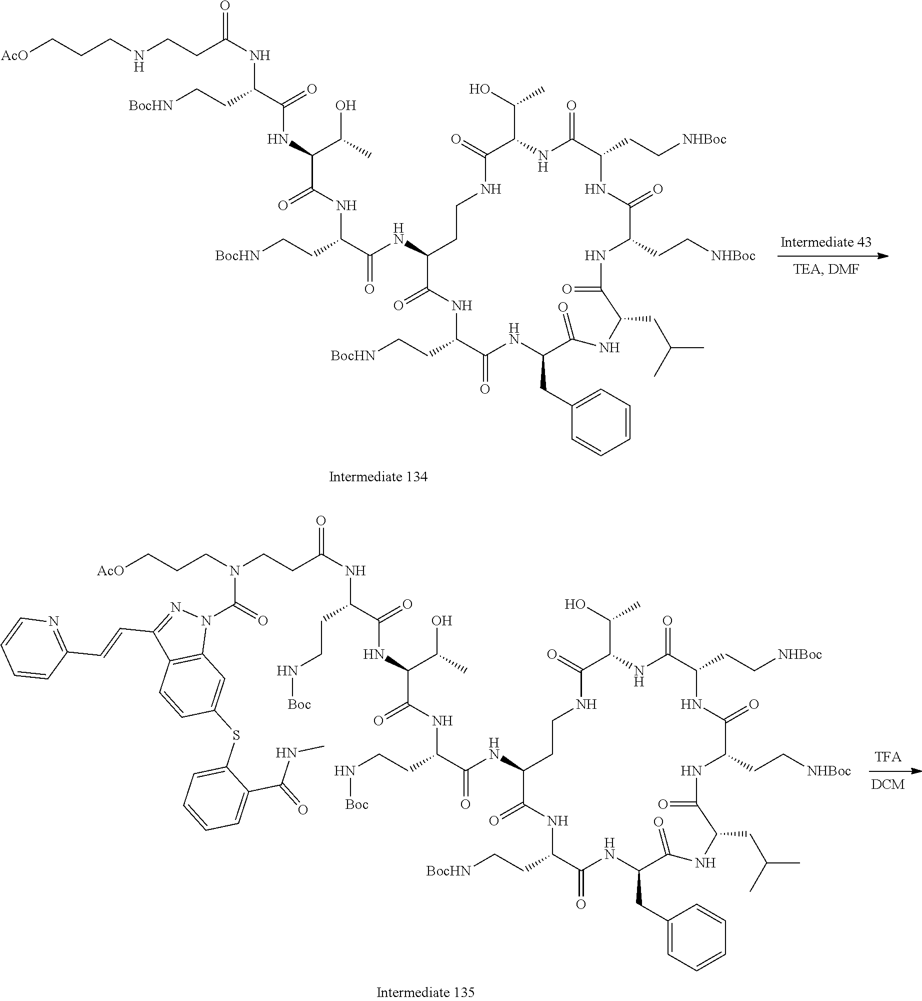

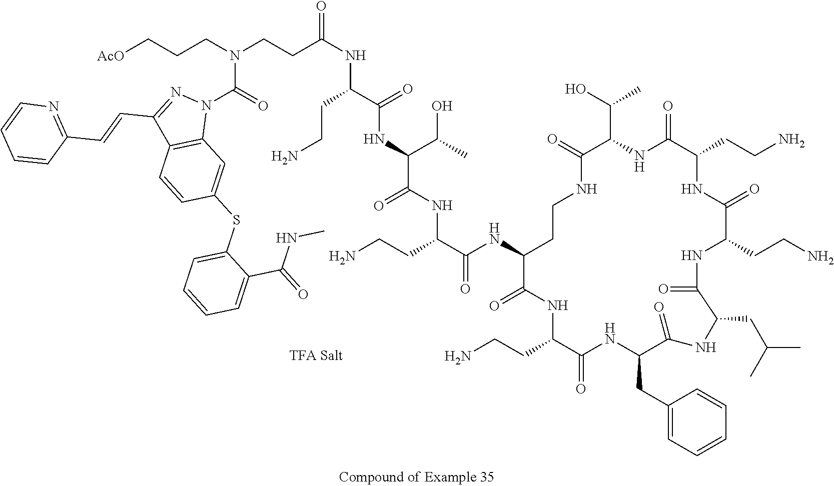 [SMILES: CNC(=O)c1ccccc1Sc1ccc2c(/C=C/c3ccccn3)nn(C(=O)N(CCCOC(C)=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 130. tert-Butyl acrylate (1.3 mL, 9 mmol) was added at 0° C. to 3-aminopropan-1-ol (0.69 mL, 9 mmol). After the mixture was stirred at r.t. o.n., benzyl (2,5-dioxopyrrolidin-1-yl) carbonate (2.47 g, 9.9 mmol) in THF/H₂O (45 mL/45 mL) was added at 0° C., followed by TEA (2.5 mL, 18 mmol). The reaction was stirred at r.t. for 2.5 h, extracted with MTBE (150 mL), washed with 10% KHSO₄ (aq. 30 mL), NaHCO₃ (aq. 30 mL) and brine (15 mL). The MTBE layer was dried and evaporated. The crude mixture was purified by silica gel chromatography (EA/PE=0˜50%) to give Intermediate 130 (2.5 g). MS: 338.0 [M+H]⁺.

Intermediate 131 The mixture of Intermediate 130 (2.5 g, 7.4 mmol), Ac₂O (1.12 mL, 11.1 mmol) and Py (1.2 mL, 14.8 mmol) in DCM (15 mL) was stirred at r.t. for 16 h, and then was extracted with EA (100 mL), washed with 0.1N HCl (aq. 30 mL) and brine (15 mL). The EA layer was dried (Na₂SO₄) and evaporated, and the product purified by silica gel chromatography (EA/PE 0˜50%) to give Intermediate 131 (2.5 g). MS: 402.2 [M+Na]⁺.

Intermediate 132. The mixture of Intermediate 131 (2 g, 2.3 mmol) in TFA/DCM (1 mL/8 mL) was stirred at r.t. for 5 h. Volatiles were evaporated to give crude Intermediate 132 (1.9 g). MS: 324.1 [M+H]⁺.

Intermediate 133. The mixture of crude Intermediate 132 (71 mg, 0.22 mmol), Dab(Boc)PMBN(Boc)₄ (313 mg, 0.2 mmol), HATU (91 mg, 0.24 mmol) and DIEA (0.053 mL, 0.3 mmol)) in DMF (5 mL) was stirred under Ar at r.t. o.n. The mixture was diluted with water and extracted with EA (40 mL). The organic layer was washed with H₂O (5 mL×2) and brine (5 mL). The EA layer was dried (Na₂SO₄) and evaporated. The residue was purified by C18 column chromatography (ACN/H₂O 0˜90%) to give Intermediate 133 (0.318 g). MS: 1868.6 [M+H]⁺.

Intermediate 134. The suspension of Intermediate 133 (318 mg, 0.17 mmol) and Pd/C (100 mg) in MeOH (25 mL) was degassed with H₂, then stirred under H₂ at r.t. for 4 h. The mixture was filtered and evaporated to give Intermediate 134 (275 mg). MS: 1734.8 [M+H]⁺.

Intermediate 135. The mixture of Intermediate 134 (275 mg, 0.159 mmol), Intermediate 43 (114 mg, 0.206 mmol) and DMAP (39 mg, 0.317 mmol) in NMP (5 mL) was stirred under Ar at 50° C. for 5 h. The reaction was cooled to r.t., extracted with EA (40 mL), washed with H₂O (5 mL×2) and brine (5 mL). The EA layer was dried and evaporated, and the residue was purified by C18 column chromatography ACN/H2O=0˜95%) to give Intermediate 135 (185 mg). MS: 1074.3 [M+2H]²⁺.

Compound of Example 35. The mixture of Intermediate 135 (237 mg, 0.11 mmol) in TFA/DCM (0.5 mL/5 mL) was stirred at r.t. for 1.5 h. Volatiles were removed under vacuum and the residue was purified by C18 column chromatography (ACN/H₂O 0˜60%) to afford compound of Example 35 as TFA salt (103 mg). MS: 1646.7 [M+H]⁺. NMR: 8.52 (d, J 5.6 Hz, 1H), 8.32 (t, J 8.1 Hz, 1H), 8.11 (d, J 8.3 Hz, 1H), 7.89-7.77 (m, 2H), 7.71 (t, J 6.7 Hz, 1H), 7.55 (d, J 16.9 Hz, 1H), 7.48 (s, 1H), 7.41 (d, J 6.6 Hz, 1H), 7.37 (dd, J 4.9, 2.1 Hz, 3H), 7.24-7.13 (m, 5H), 7.12-7.05 (m, 2H), 4.41 (t, J 8.2 Hz, 1H), 4.33 (ddd, J 8.9, 5.4, 2.8 Hz, 3H), 4.19 (d, J 4.4 Hz, 1H), 4.15-3.99 (m, 8H), 3.87 (s, 2H), 3.72 (s, 2H), 3.55 (s, 2H), 2.94 (dq, J 19.6, 11.0, 9.3 Hz, 12H), 2.68-2.58 (m, 3H), 2.55 (s, 3H), 2.15-1.99 (m, 6H), 1.98-1.47 (m, 13H), 1.40-1.19 (m, 2H), 1.01 (dd, J 8.9, 6.4 Hz, 7H), 0.60 (d, J 5.7 Hz, 3H), 0.53 (d, J 6.0 Hz, 3H).

Optionally, the TFA salt of the compound of Example 35 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product can be optionally recrystallized, for example from an alcohol-containing media, such as EtOH-EtOAc, or isopropanol-water, or similar solvent system.

### Example 36

### Synthesis of the Compound of Example 36

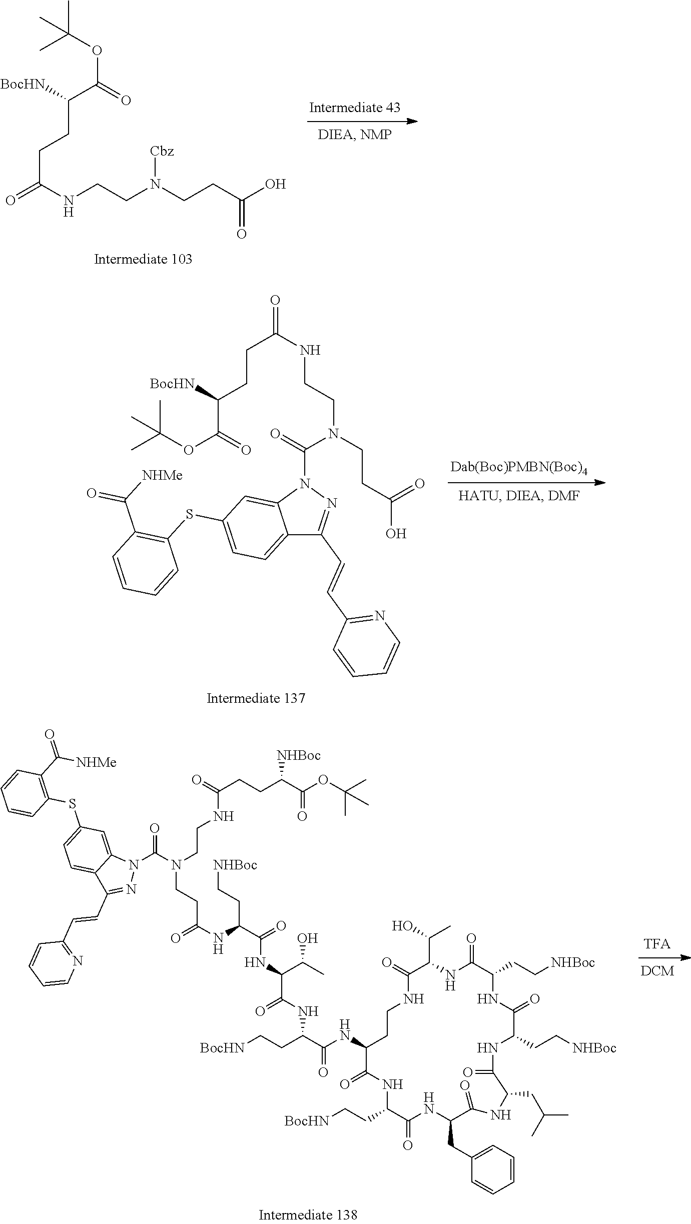

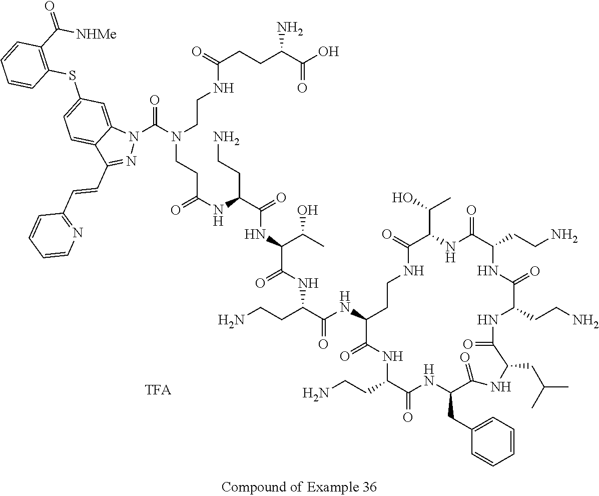 [SMILES: CNC(=O)c1ccccc1Sc1ccc2c(/C=C/c3ccccn3)nn(C(=O)N(CCNC(=O)CC[C@H](N)C(=O)O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 137. The mixture of Intermediate 103 (0.104 g, 0.24 mmol), Intermediate 43 (0.137 g, 0.24 mmol) and DIEA (0.064 mL, 0.36 mmol) in NMP (2 mL) was stirred at r.t. under Ar for 16 h. The mixture was diluted with water and extracted with EA (30 mL). The organic layer was washed with brine (5 mL×3), dried (Na₂SO₄) and evaporated under vacuum. The residue was purified by C18 column chromatography ACN/H₂O 0˜90%) to give Intermediate 137 (0.08 g). MS: 830.2 [M+H]⁺.

Intermediate 138 The mixture of Intermediate 137 (0.08 g, 0.096 mmol), Dab(Boc)PMBN(BoC)₄ (0.151 g, 0.096 mmol), HATU (0.044 g, 0.116 mmol) and DIEA (0.034 mL, 0.193 mmol) in DMF (3 mL) was stirred at 25° C. under Ar for 5 h. The mixture was diluted with water and extracted with EA (50 mL). The organic layer was washed with H₂O (5 mL×2) and brine (5 mL). The EA layer was dried and evaporated. The residue was purified by C18 column chromatography (ACN/H₂O 0˜90%) to give Intermediate 138 (0.11 g).

Compound of Example 36. The mixture of Intermediate 138 (0.11 g, 0.046 mmol) in TFA/DCM (1 mL/3 mL) was stirred at r.t. for 1 h. Volatiles were removed under vacuum and the residue was purified by C18 column chromatography (ACN/H₂O 0˜50%) to give the compound of Example 36 (70 mg). MS: 1719.7 [M+H]⁺. NMR: 8.63 (d, J 5.9 Hz, 1H), 8.54-8.44 (m, 1H), 8.29 (d, J 8.3 Hz, 1H), 8.00-7.91 (m, 1H), 7.86 (ddd, J 7.4, 5.9, 1.2 Hz, 1H), 7.68 (d, J 15.6 Hz, 1H), 7.54 (d, J 22.4 Hz, 2H), 7.45 (d, J 1.5 Hz, 3H), 7.28 (dq, J 9.6, 7.2 Hz, 4H), 7.20-7.13 (m, 2H), 4.49 (t, J 8.3 Hz, 1H), 4.42 (dt, J 10.1, 5.2 Hz, 3H), 4.27-4.07 (m, 9H), 3.83 (s, 1H), 3.68 (s, 2H), 3.35 (s, 1H), 3.24 (dt, J 14.6, 7.7 Hz, 1H), 3.10-2.93 (m, 11H), 2.85-2.67 (m, 3H), 2.63 (d, J 2.8 Hz, 4H), 2.15 (tt, J 12.1, 6.3 Hz, 7H), 1.46-1.28 (m, 2H), 1.09 (dd, J 12.3, 6.4 Hz, 6H), 0.68 (d, J 4.8 Hz, 3H), 0.61 (d, J 5.4 Hz, 3H).

Optionally, the TFA salt of the compound of Example 36 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product can be optionally recrystallized, for example from an alcohol-containing media, such as EtOH-EtOAc, or isopropanol-water, or similar solvent system.

### Example 37

### Synthesis of the Compound of Example 37

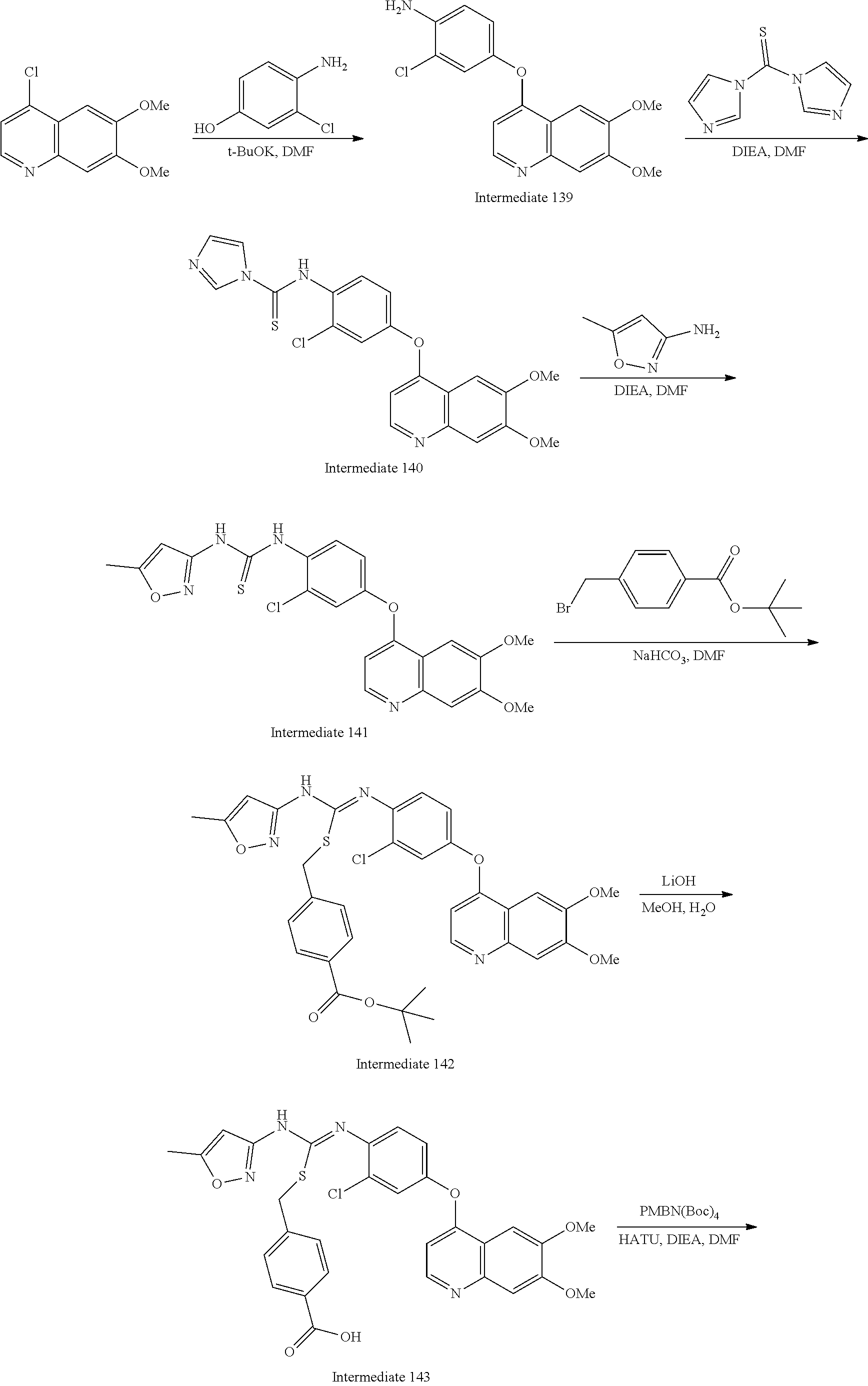

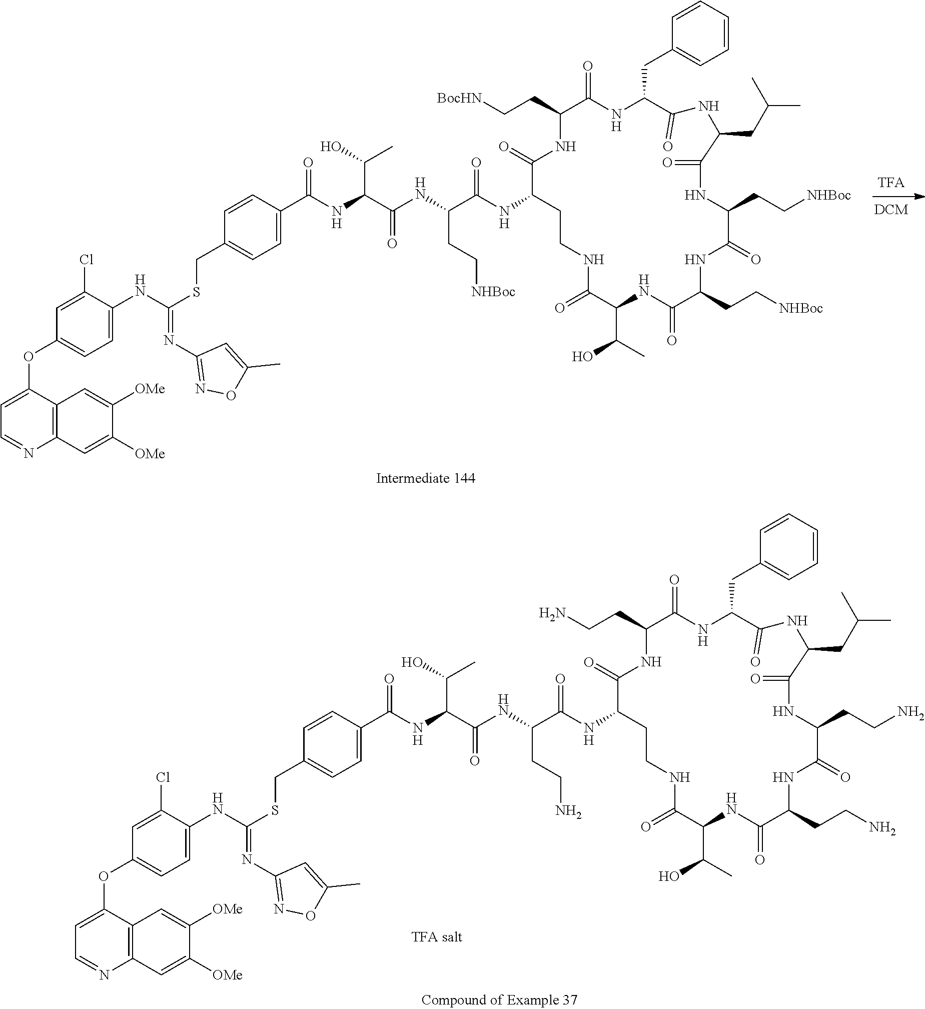

Intermediate 139. A mixture of 4-chloro-6,7-dimethoxyquinoline (2.24 g, 10 mmol), 4-amino-3-chlorophenol (1.72 g, 12 mmol) and t-BuOK (1.35 g, 12 mmol) in DMF (30 mL) was stirred at 120 under Ar for 8 h, then it was cooled to r.t., and diluted with water (50 mL). The mixture was extracted with DCM (80 mL×3. The organic layer was washed with H₂O (30 mL×2) and brine (30 mL). The DCM layer was dried, evaporated, and purified by C18 column chromatography (ACN/H₂O 0˜100%) to give Intermediate 139 (2.53 g). MS: 331.27 [M+H]⁺.

Intermediate 140 The mixture of Intermediate 139 (3.1 g, 9.4 mmol), di(1H-imidazol-1-yl)methanethione (2 g, 11.2 mmol) and DIEA (2.2 mL, 12.2 mmol) in DMF (20 mL) was stirred at 40° C. under Ar for 8 h. It was then cooled to r.t. and quenched with H₂O (50 mL). The resulting mixture was extracted with EA (80 mL×3), which was washed with H₂O (30 mL×2) and brine (30 mL). The organic layer was dried (Na₂SO₄) and evaporated under vacuum. The residue was purified by C18 column chromatography (gradient ACN/H2O=0˜100%) to give Intermediate 140 (4 g). MS: 441.26 [M+H]⁺.

Intermediate 141. Intermediate 140 (4 g, 9.1 mmol), 3-amino-5-methyl-isoxazole (1.6 g, 16.3 mmol) and DIEA (3.2 mL, 18.1 mmol) were mixed in DMF (30 mL). The reaction was stirred at 40° C. under Ar for 16 h, then cooled to r.t. and quenched with H₂O (50 mL). The resulting mixture was extracted with EA (80 mL×3), which was washed with H₂O (30 mL×2) and brine (30 mL). The organic layer was dried (Na₂SO₄), filtered, and evaporated. The residue was purified by silica gel chromatography (EA/PE 10˜100% to MeOH/DCM=3˜12%) to give Intermediate 141 (2.35 g). MS: 471.26 [M+H]⁺.

Intermediate 142. Intermediate 141 (471 mg, 1 mmol), tert-butyl 4-(bromomethyl)benzoate (298 mg, 1.1 mmol) and NaHCO₃ (126 mg, 1.5 mmol) were mixed in DMF (5 mL), and stirred at 75° C. for 5.5 h. Then it was cooled down to r.t., quenched with H₂O (10 mL), and extracted with EA (20 mL×3), washed with H₂O (10 mL×2) and brine (10 mL). The EA layer was dried, evaporated, and the residue purified by silica gel chromatography (EA/PE 10˜100%) to give Intermediate 142 (427 mg). MS: 661.21 [M+H]⁺.

Intermediate 143. A mixture of Intermediate 142 (400 mg, 0.6 mmol) and LiOH H₂O (46 mg, 1.1 mmol) in MeOH/H₂O (3 mL/5 mL) was stirred at r.t. o.n. The mixture was neutralized with AcOH and extracted with EA (30 mL). The organic layer was washed with brine (10 mL), dried (Na₂SO₄) and evaporated under vacuum. The residue was purified by C18 column chromatography ACN/H2O=0˜70%) to give Intermediate 143 (0.25 g) MS: 605.21 [M+H]⁺.

Intermediate 144. The mixture of Intermediate 143 (240 mg, 0.4 mmol), PMBN(Boc)₄ (595 mg, 0.44 mmol), HATU (182 mg, 0.48 mmol) and DIEA (0.142 mL, 0.8 mmol) in DMF (6 mL) was stirred at 25° C. under Ar for 5 h. The mixture was diluted with water and extracted with EA (50 mL). The organic layer was washed with H₂O (5 mL×2) and brine (5 mL), dried (Na₂SO₄) and evaporated under vacuum. The residue was purified by C18 column chromatography ACN/H₂O 0˜90%) to give Intermediate 144 (145 mg). MS: 1950.57 [M+2H]²⁺.

Compound of Example 37. The solution of Intermediate 144 (0.11 g, 0.056 mmol) in TFA/DCM (1 mL/3 mL) was stirred at r.t. for 1 h. Volatiles were removed under vacuum and the residue was purified by C18 column chromatography (ACN/H₂O 0˜70%) to afford the compound of Example 37 as TFA salt (108 mg). MS: 1549.4 [M+H]⁺. NMR: 8.51 (d, J 6.8 Hz, 1H), 7.76 (d, J 8.0 Hz, 2H), 7.72 (s, 1H), 7.57-7.47 (m, 3H), 7.31-7.18 (m, 4H), 7.11 (d, J 7.1 Hz, 2H), 4.43 (s, 4H), 4.11 (s, 7H), 4.03 (d, J 5.9 Hz, 4H), 3.97 (s, 4H), 2.97 (d, J 35.0 Hz, 10H), 2.75 (d, J 38.1 Hz, 3H), 2.21 (s, 2H), 2.09 (d, J 43.4 Hz, 6H), 1.83 (s, 6H), 1.33 (d, J 21.5 Hz, 2H), 1.22 (d, J 6.4 Hz, 4H), 1.04 (d, J 6.5 Hz, 3H), 0.66 (s, 3H), 0.57 (s, 3H).

Optionally, the TFA salt of the compound of Example 37 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 38

### Synthesis of the Compound of Example 38

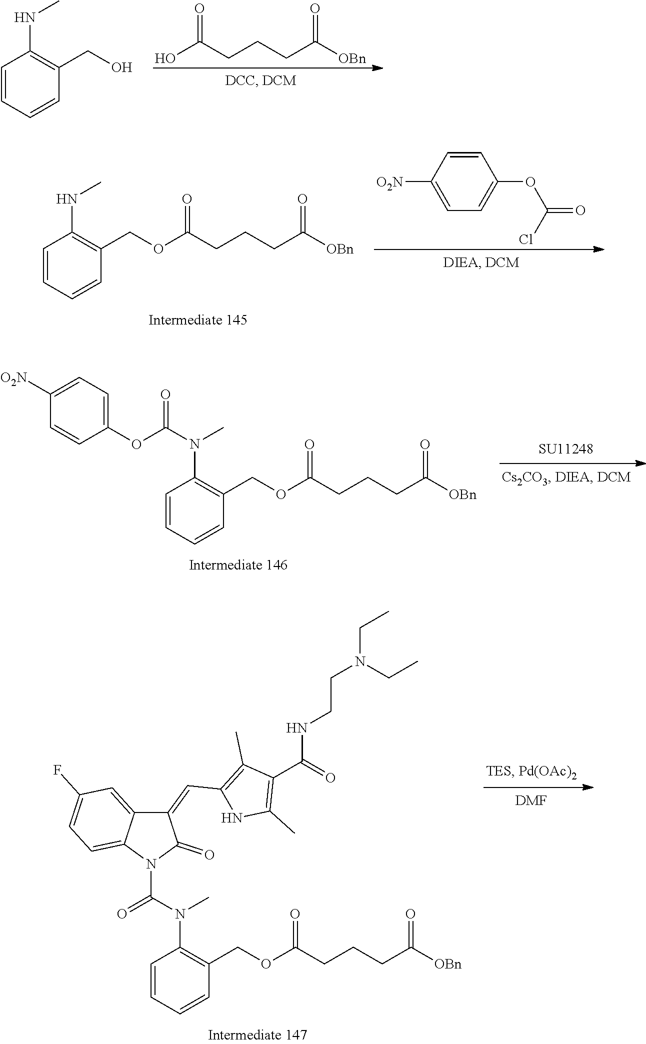

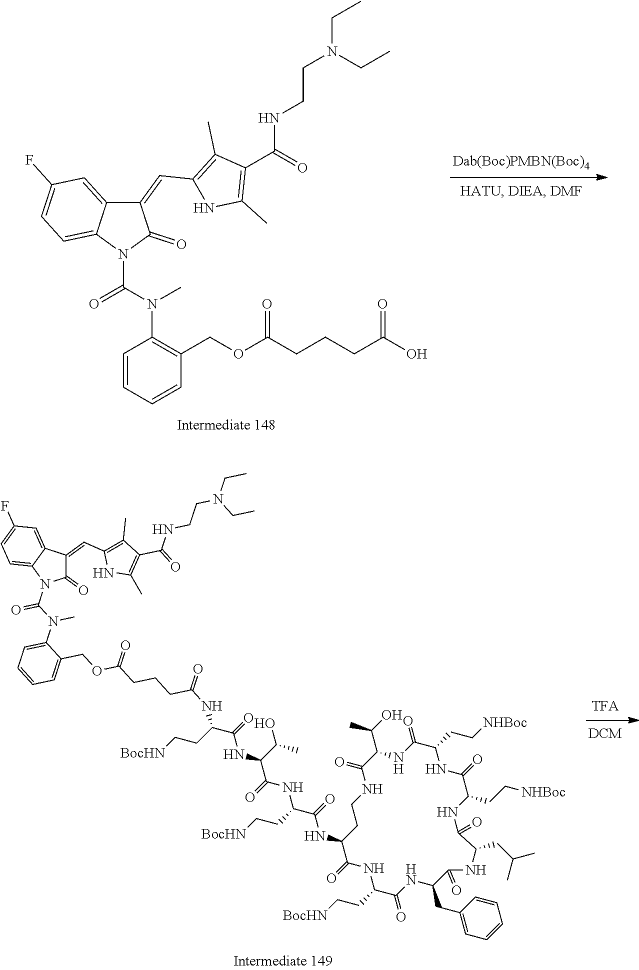

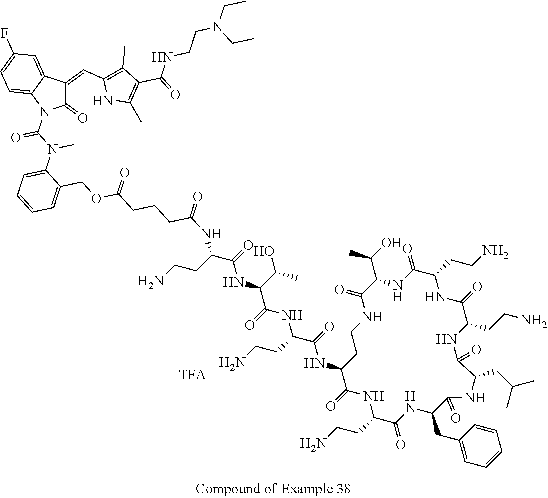 [SMILES: CCN(CC)CCNC(=O)c1c(C)[nH]c(/C=C2\C(=O)N(C(=O)N(C)c3ccccc3COC(=O)CCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C]

Intermediate 145. A solution of (2-(methylamino)phenyl)methanol (800 mg, 5.6 mmol), 5-(benzyloxy)-5-oxopentanoic acid (1.2 g, 5.6 mmol), DCC (1.1 g, 5.6 mmol) in DCM (100 mL) was stirred at r.t. for 16 h, then concentrated, dissolved with 200 mL of EA, and then washed with H₂O (20 mL×2) and brine (20 mL×2), and the EA layer was dried and evaporated under vacuum. The residue was purified by silica gel column to give Intermediate 145 (588 mg). MS: 342.0 [M+H]⁺.

Intermediate 146 To a solution of Intermediate 145 (341 mg, 1.0 mmol) and p-nitro-phenyl chloroformate (303 mg, 1.5 mmol) in DCM (15 mL) was added DIEA (280 μL, 2.0 mmol) at 0° C. The mixture was stirred at r.t. for 16 h, then concentrated under vacuum, diluted with 150 mL of EA, and then washed with H₂O (20 mL×2) and brine (20 mL×2). The EA layer was dried and concentrated. The residue was purified by silica gel column to give Intermediate 146 (255 mg). MS: 529.2 [M+Na]⁺.

Intermediate 147 A mixture of Intermediate 146 (360 mg, 0.7 mmol), SU11248 ((Z)—N-(2-(diethylamino)ethyl)-5-((5-fluoro-2-oxoindolin-3-ylidene)methyl)-2,4-dimethyl-1H-pyrrole-3-carboxamide; 200 mg, 0.5 mmol), Cs₂CO₃ (285 mg, 0.75 mmol), TEA (17 μL, 0.12 mmol) in DCM (10 mL) was stirred at r.t. for 4 h. The reaction mixture was diluted with 200 mL of EA, and washed with H₂O (20 mL×2) and brine (20 mL×2), The EA layer was dried and concentrated. The crude was purified by silica gel column to give Intermediate 147 (211 mg). MS: 766.3 [M+H]⁺.

Intermediate 148 A mixture of Intermediate 147 (105 mg, 0.14 mmol), Pd(OAc)₂ (4 mg, 0.02 mmol), TES (30 mg, 0.28 mmol), in DMF (10 mL) was stirred at r.t. for 2 h. The mixture was concentrated, diluted with 100 mL of EA, and then washed with H₂O (10 mL×2), brine (10 mL×2), and the EA layer was dried and concentrated. The crude was purified by reverse phase system to give Intermediate 148 (55 mg). MS: 676.3 [M+H]⁺.

Intermediate 149 A mixture of Intermediate 148 (45 mg, 0.07 mmol), Dab(Boc)-PMBN(Boc)₄ (125 mg, 0.07 mmol), DIEA (28 μL, 0.13 mmol), HATU (33 mg, 0.08 mmol) in THE (10 mL) was stirred at r.t. for 16 h. The reaction mixture was diluted with 100 mL of EA, and then washed with H₂O (10 mL×2) and brine (10 mL×2), and the EA layer was dried and concentrated. The crude was purified by reverse phase system to give Intermediate 149 (121 mg).

Compound of Example 38. A mixture of Intermediate 149 (115 mg, 0.05 mmol) in TFA/DCM (1 mL/10 mL) was stirred at r.t. for 2 h. The mixture was concentrated, and the residue was purified by prep-HPLC to afford the compound of Example 38 as TFA salt (34 mg). MS: 1720.7 [M+H]⁺. NMR: 7.35-7.13 (m, 4H), 7.07-6.89 (m, 11H), 6.78-6.70 (m, 1H), 4.28-3.84 (m, 12H), 3.50-3.43 (m, 2H), 3.24-2.95 (m, 10H), 2.89-2.47 (m, 13H), 2.23-1.41 (m, 23H), 1.23-1.03 (m, 7H), 0.87-0.85 (m, 6H), 0.49-0.32 (m, 7H).

### Example 39

### Synthesis of the Compound of Example 39

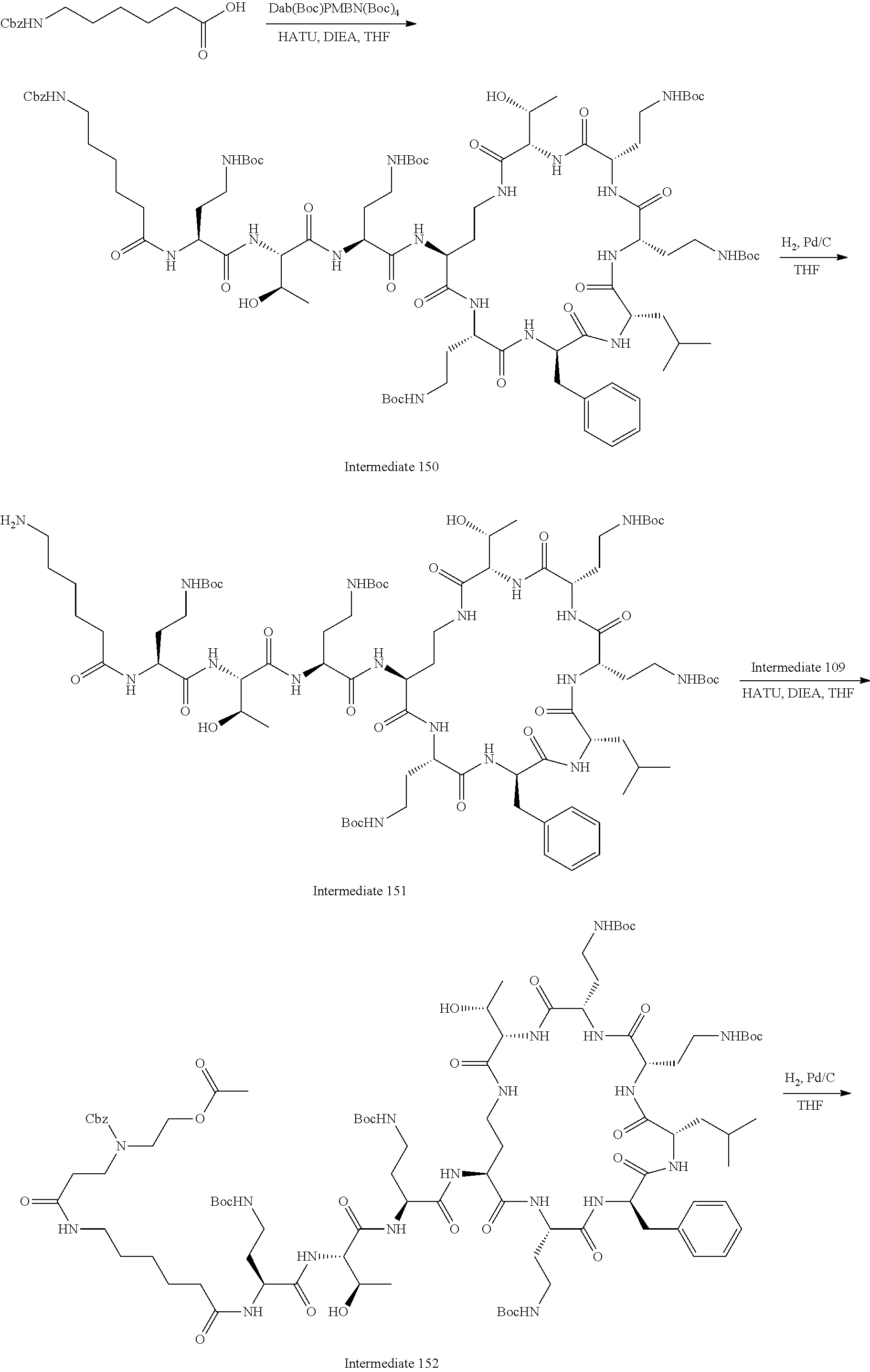

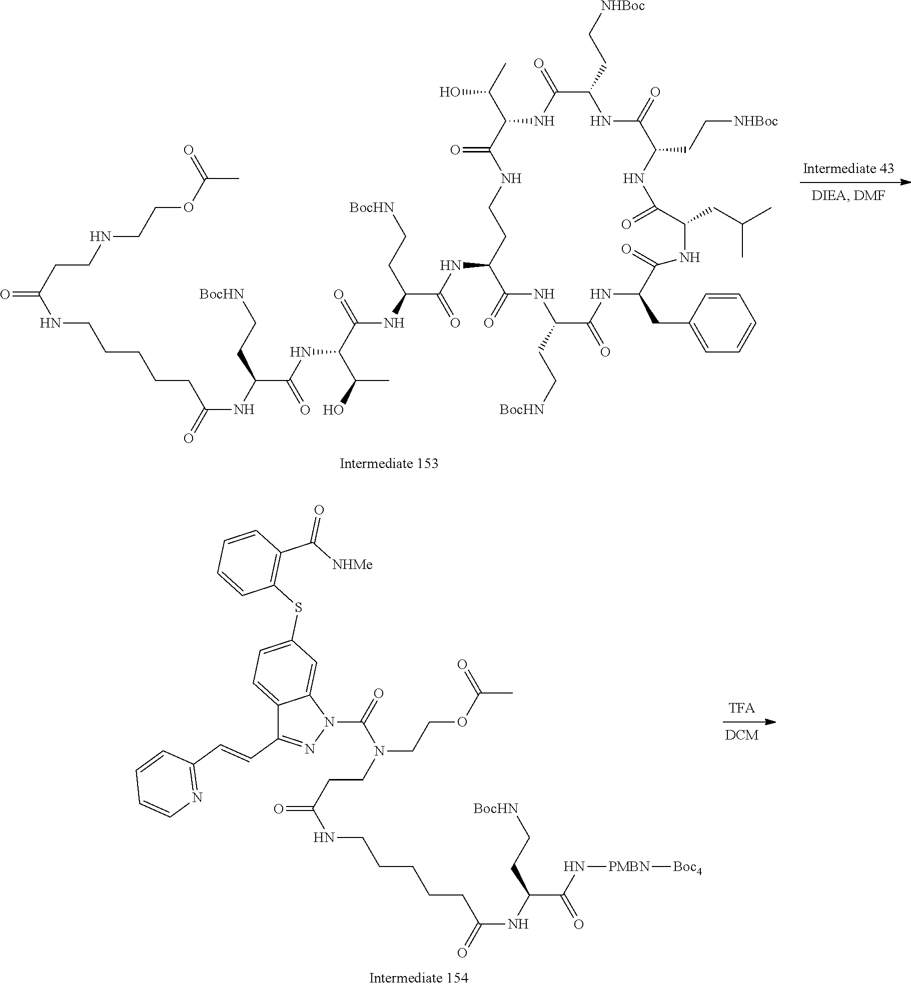

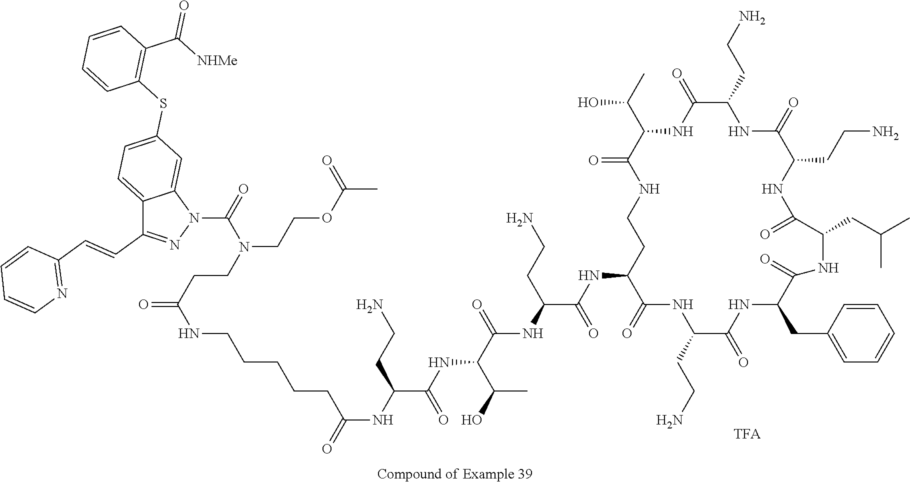 [SMILES: CNC(=O)c1ccccc1Sc1ccc2c(/C=C/c3ccccn3)nn(C(=O)N(CCOC(C)=O)CCC(=O)NCCCCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 150. A mixture of 6-(((benzyloxy)carbonyl)amino)hexanoic acid (155 mg, 0.58 mmol), Dab(Boc)PMBN(Boc)₄ (600 mg, 0.38 mmol), DIEA (140 μL, 0.76 mmol), HATU (288 mg, 0.76 mmol) in THE (50 mL) was stirred at r.t. for 5 h. The reaction mixture was diluted with EA (200 mL), and then washed with H₂O (20 mL×2) and brine (20 mL×2), and the EA layer was dried and concentrated. The crude was purified by reverse phase system to give Intermediate 150 (511 mg). MS: 1810.5 [M+H]⁺.

Intermediate 151 A suspension of Intermediate 150 (400 mg, 0.22 mmol), Pd/C (603 mg) in THE (25 mL) was stirred under H₂ at r.t. for 6 h. The mixture was filtered, and concentrated to give crude Intermediate 151 (356 mg). MS: 1676.8 [M+H]⁺.

Intermediate 152. A mixture of Intermediate 151 (356 mg, 0.2 mmol), Intermediate 109 (94 mg, 0.3 mmol), DIEA (75 μL, 0.4 mmol), HATU (152 mg, 0.4 mmol) in THE (20 mL) was stirred at r.t. for 5 h. The reaction mixture was diluted with 200 mL of EA, and then washed with H₂O (20 mL×2) and brine (10 mL×2). The EA layer was dried (Na₂SO₄) and evaporated under vacuum. The crude was purified by reverse phase system to give Intermediate 152 (358 mg). MS: 1968.6 [M+H]⁺.

Intermediate 153. A mixture of Intermediate 152 (250 mg, 0.13 mmol), Pd/C (40 mg) in 20 mL of THE was degassed and stirred under H₂ at r.t. for 15 h. The mixture was filtered, concentrated to give Intermediate 153 (242 mg). MS: 1833.9 [M+H]⁺.

Intermediate 154. Intermediate 153 (251 mg, 0.14 mmol), Intermediate 43 (151 mg, 0.28 mmol), DIEA (65 μL, 0.35 mmol) in 10 mL of DMF was stirred at r.t. for 16 h. The reaction mixture was diluted with 200 mL of EA, and then washed with H₂O (10 mL×2) and brine (10 mL×2), and the EA layer was dried (Na₂SO₄) and evaporated under vacuum. The crude product was purified by reverse phase system to give Intermediate 154 (153 mg).

Compound of Example 39. Intermediate 154 (0.10 g, 0.07 mmol) in TFA/DCM (1 mL/10 mL) was stirred at r.t. for 2 h. The mixture was concentrated and purified by Prep-HPLC to afford the compound of Example 39 as TFA salt (38 mg). MS: 1745.8 [M+H]⁺. NMR: 8.33-8.13 (m, 2H), 7.96-7.85 (m, 1H), 7.74-7.09 (m, 15H), 4.45-4.07 (m, 12H), 3.90-3.82 (m, 1H), 3.22-3.19 (m, 1H), 3.02-2.40 (m, 21H), 2.16-2.69 (m, 18H), 1.37-0.91 (m, 17H), 0.69-0.52 (m, 9H).

Optionally, the TFA salt of the compound of Example 39 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 40

### Synthesis of the Compound of Example 40

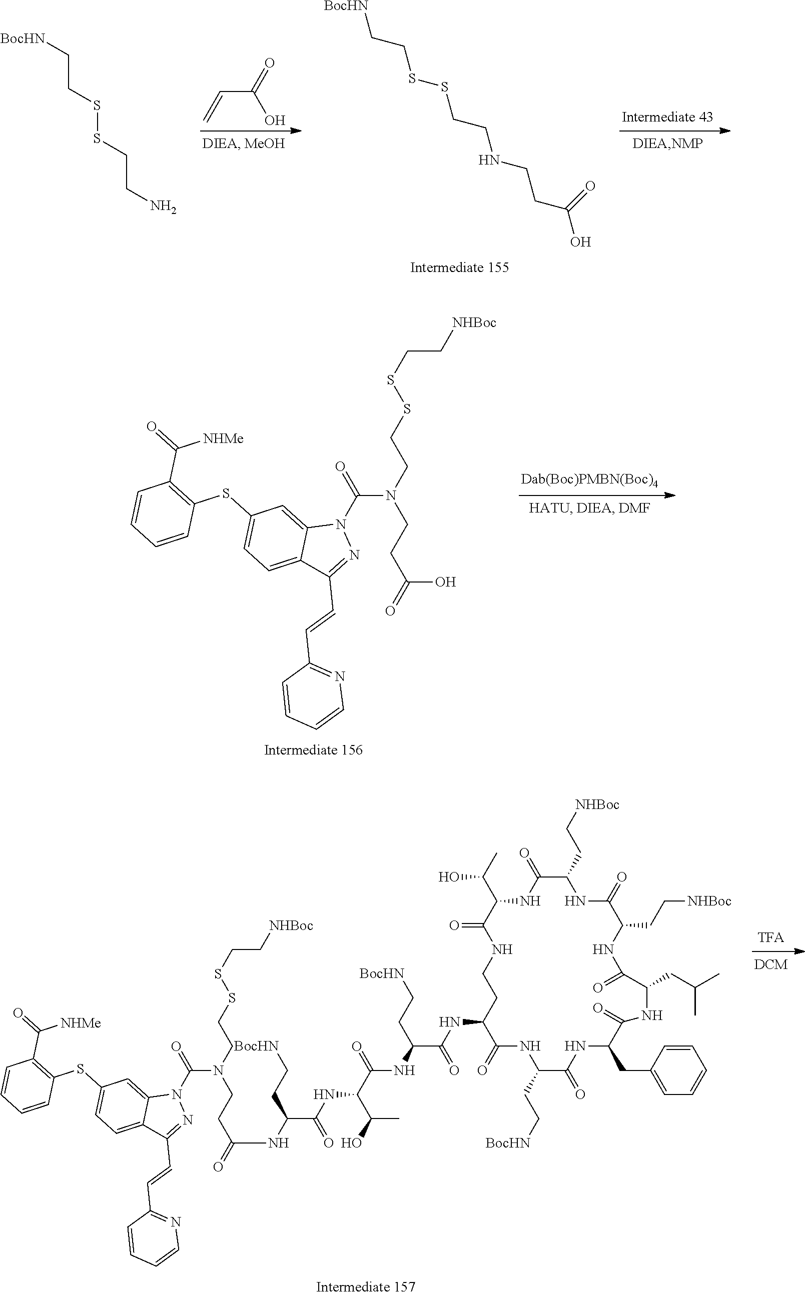

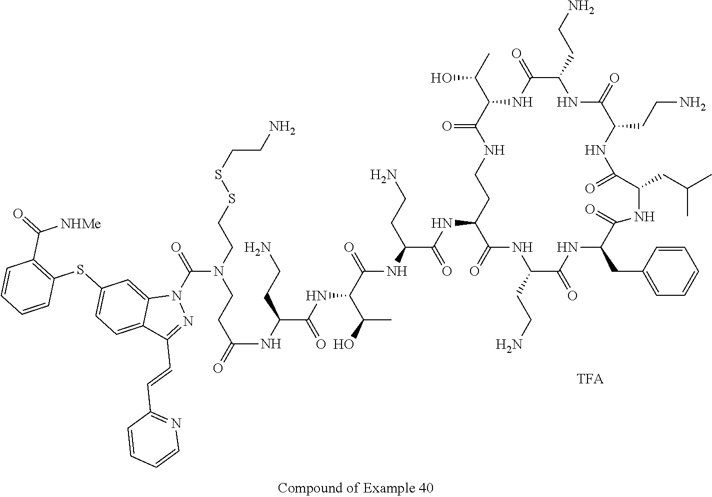 [SMILES: CNC(=O)c1ccccc1Sc1ccc2c(/C=C/c3ccccn3)nn(C(=O)N(CCSSCCN)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 155. A mixture of ter.t-butyl (2-((2-aminoethyl)disulfanyl)ethyl)carbamate (300 mg, 1.1 mmol), acrylic acid (85 mg, 1.1 mmol), DIEA (1 mL, 5.5 mmol) in 10 mL of MeOH was stirred at r.t. for 6 h. The mixture was concentrated, and the crude material was purified by HPLC (C18 column), to afford Intermediate 155 (288 mg). MS: 325.1 [M+H]⁺.

Intermediate 156 Intermediate 155 (194 mg, 0.6 mmol), Intermediate 43 (300 mg, 0.6 mmol), DIEA (221 μL, 1.2 mmol) in 10 mL of NMP was stirred at r.t. for 16 h. The reaction mixture was diluted with 200 mL of EA, and then washed with H₂O (20 mL×2) and brine (20 mL×2), and the EA layer dried and concentrated. The crude was purified by C18 column chromatography to give Intermediate 156 (219 mg). MS: 737.2 [M+H]⁺.

Intermediate 157. Intermediate 156 (141 mg, 0.19 mmol), Dab(Boc)PMBN(Boc)₄ (300 mg, 0.19 mmol), HATU (115 mg, 0.3 mmol), DIEA (70 μL, 0.38 mmol) in THE (30 mL) were stirred at r.t. for 5 h. The reaction mixture was diluted with 300 mL of EA, and then washed with H₂O (20 mL×2), brine (20 mL×2), dried (Na₂SO₄) and evaporated under vacuum. The crude product was purified by C18 column chromatography to give Intermediate 157 (211 mg).

Compound of Example 40. Intermediate 157 (200 mg, 0.09 mmol) in TFA/TES/DCM (0.5 mL/10 mL) was stirred at r.t. for 2 h. The mixture was concentrated and purified by Prep-HPLC to afford the compound of Example 40 as TFA salt (43 mg). MS: 1682.7 [M+H]⁺.

NMR: 8.55 (d, J 5.2 Hz, 1H), 8.27 (t, J 8.4 Hz, 1H), 8.06 (d, J 8.0 Hz, 1H), 7.93 (d, J 8.4 Hz, 1H), 7.84-7.11 (m, 14H), 4.48-4.36 (m, 4H), 4.25-4.05 (m, 8H), 3.87-3.68 (m, 4H), 3.24-3.54 (m, 24H), 2.14-1.73 (m, 12H), 1.41-1.26 (m, 2H), 1.05 (dd, J 13.2, 6.8 Hz, 6H), 0.66-0.57 (m, 7H).

Optionally, the TFA salt of the compound of Example 40 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 41

### Synthesis of the Compound of Example 41

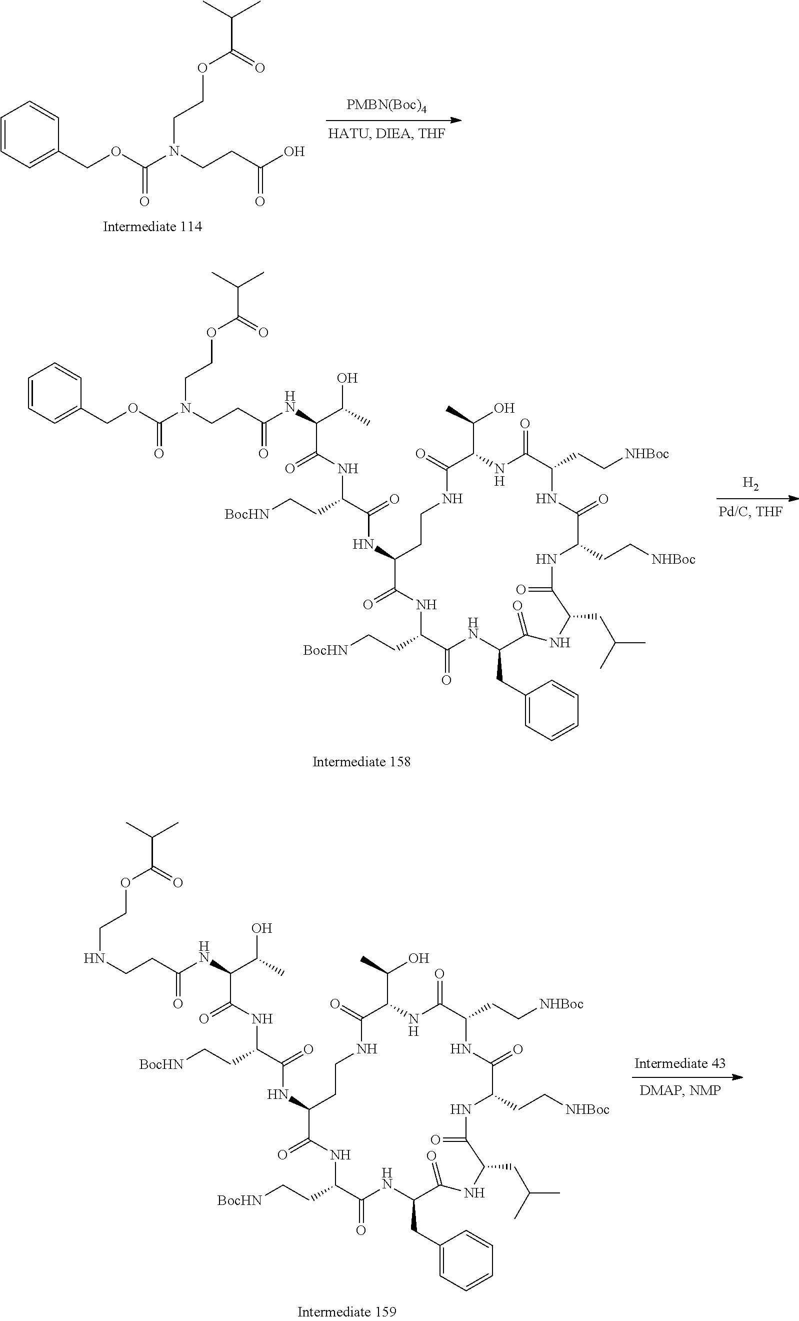

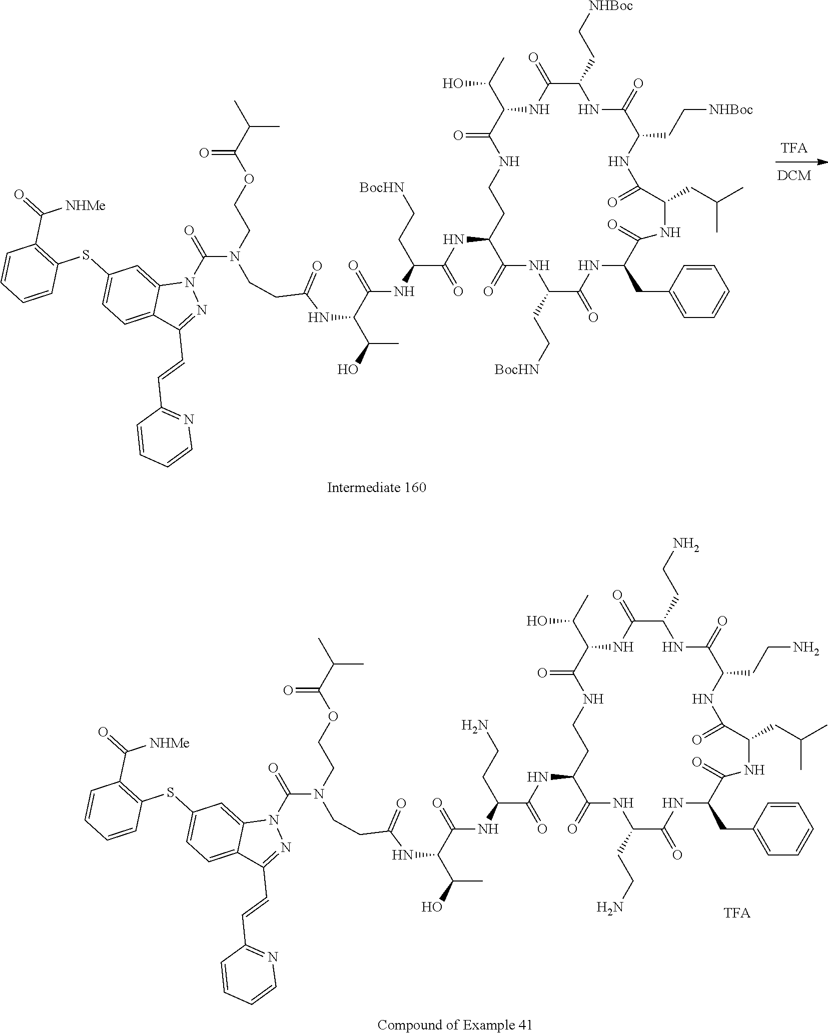

Compound of Example 41. The compound of Example 41 as TFA salt was synthesized according to the procedures described for the preparation of the compound of Example 31 except that the Intermediate 114 was coupled with PMBN(Boc)₄ instead of Dab(Boc)PMBN(Boc)₄ (used in the preparation of the compound of Example 31). White solid. MS: 1560.64 [M+H]⁺. NMR: 8.55 (d, J 5.6 Hz, 1H), 8.38-8.15 (m, 2H), 7.92-7.38 (m, 9H), 7.34-7.04 (m, 6H), 4.53-4.00 (m, 9H), 3.94-3.56 (m, 2H), 3.22-3.20 (m, 1H), 3.11-3.53 (m, 16H), 2.20-1.67 (m, 10H), 1.38-0.87 (m, 12H), 0.80-0.41 (m, 13H).

Optionally, the TFA salt of the compound of Example 41 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chem. Education. 2005, vol. 82, p. 575); or by dissolution of the TFA salt in aq. media, addition of an excess of aq. HCl, followed by lyophilization or direct evaporation of a solution under vacuum. The resulting solid product is optionally recrystallized, for example from an alcohol-containing media, such as EtOH-EtOAc, or isopropanol-water, or similar solvent system.

### Example 42

### Synthesis of the Compound of Example 42

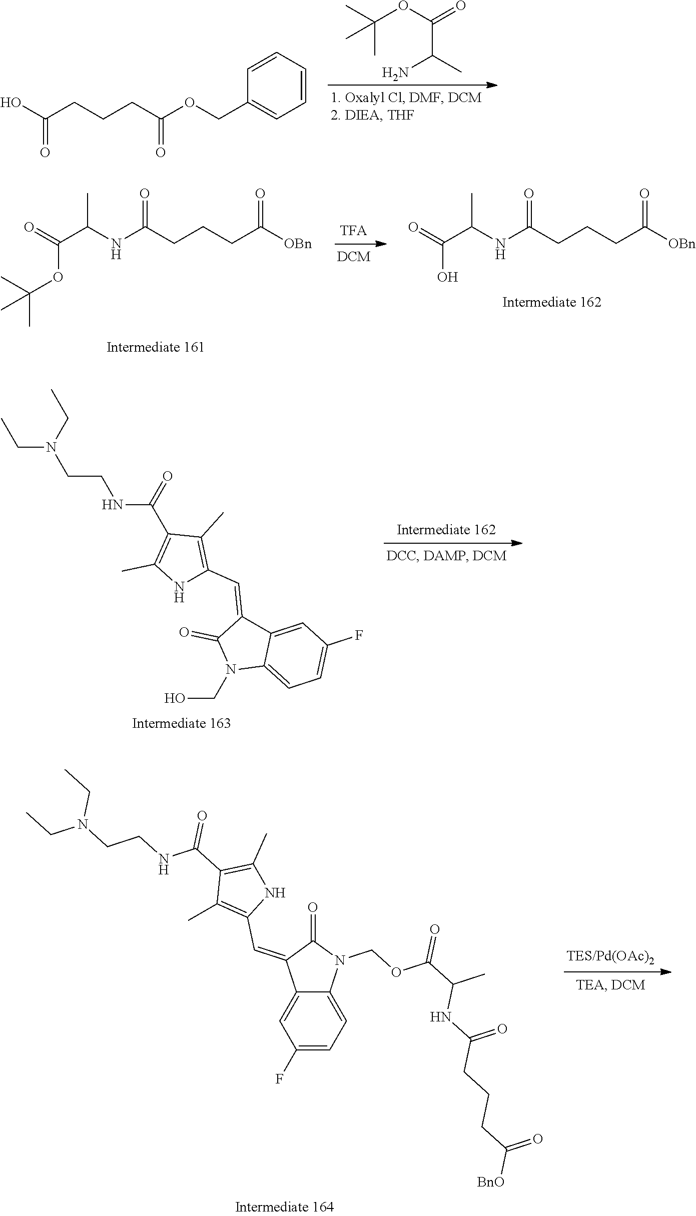

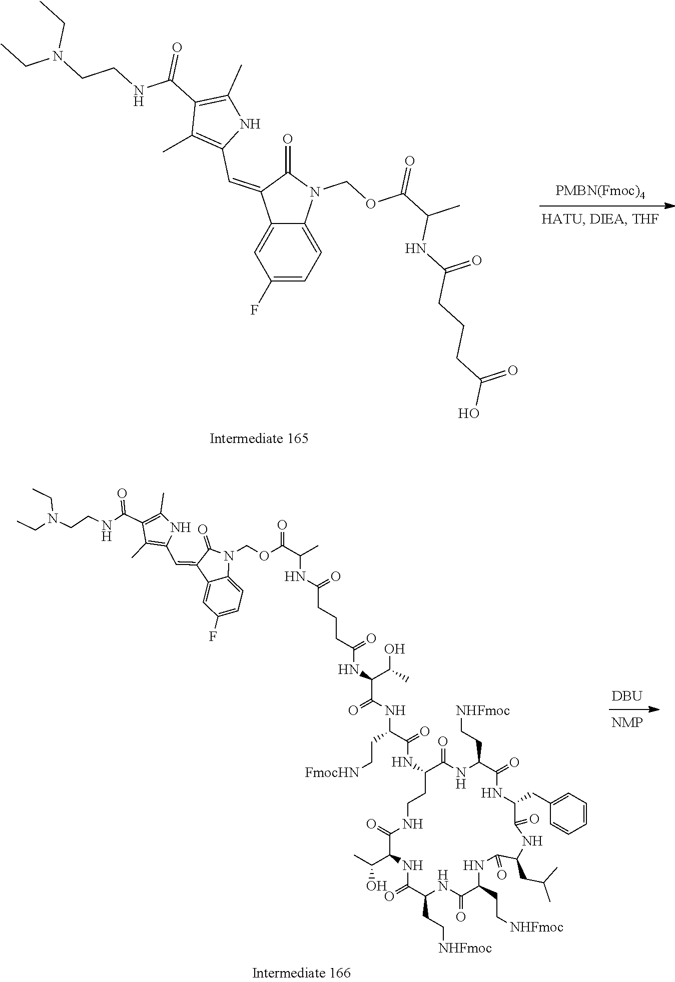

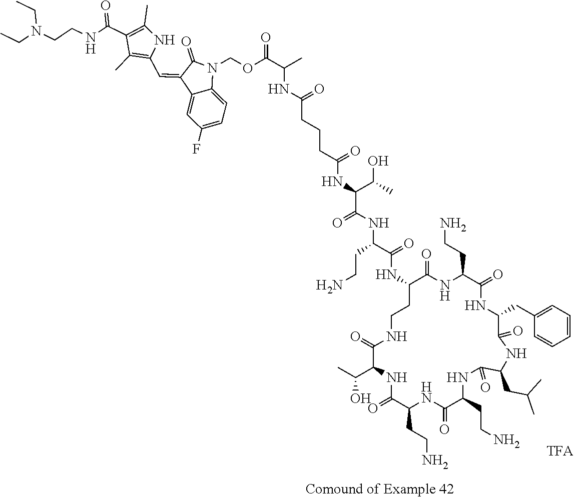 [SMILES: CCN(CC)CCNC(=O)c1c(C)[nH]c(/C=C2\C(=O)N(COC(=O)C(C)NC(=O)CCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C]

Intermediate 161. A solution of 5-(benzyloxy)-5-oxopentanoic acid (600 mg, 5.6 mmol), oxalyl chloride (476 μL, 3.8 mmol), DMF (5 drops) in 25 mL of DCM was stirred at r.t. for 2 h, concentrated, and dissolved in THF. Crude tert-butyl 2-aminopropanoate (400 mg) and DIEA (940 μL, 5.1 mmol) in 25 mL of THF was added at 0° C. The mixture was stirred at r.t. for 16 h, concentrated, diluted with 200 mL of EA, washed with H₂O (20 mL×2) and brine (20 mL×2). The volatiles were evaporated. The crude was purified by silica gel column to give Intermediate 161 (400 mg). MS: 350.2 [M+H]⁺.

Intermediate 162 The solution of Intermediate 161 (400 mg, 1.2 mmol) in TFA/DCM (2 mL/4 mL) was stirred at r.t. for 5 h, and evaporated under vacuum. The crude material was purified by silica gel column to give Intermediate 162 (355 mg). MS: 294.2 [M+H]⁺.

Intermediate 164 A mixture of Intermediate 162 (73 mg, 0.25 mmol), Intermediate 163 (111 mg, 0.25 mmol, made as described in EP3252048A¹, p. 8), DCC (61 mg, 0.28 mmol), and DMAP (7.0 mg, 0.028 mmol) in 15 mL of DCM was stirred at r.t. for 16 h. The reaction mixture was diluted with 100 mL of DCM, and then washed with H₂O (10 mL×2) and brine (10 mL×2), and the DCM layer was dried and concentrated. The crude was purified by reverse phase system to give Intermediate 164 (115 mg). MS: 704.3 [M+H]⁺.

Intermediate 165 Intermediate 164 (100 mg, 0.14 mmol), Pd(OAc)₂ (4 mg, 0.02 mmol), TES (49 mg, 0.42 mmol), and TEA (14 mg, 0.13 mmol) in 10 mL of DCM were stirred at r.t. for 2 h, concentrated, diluted with 100 mL of EA, and then washed with H₂O (10 mL×2), brine (10 mL×2), dried and concentrated. The crude was purified by reverse phase system to give Intermediate 165 (45 mg). MS: 614.3 [M+H]⁺.

Intermediate 166 A mixture of Intermediate 165 (33 mg, 0.05 mmol), PMBN(Fmoc)₄ (100 mg, 0.05 mmol), DIEA (20 μL, 0.10 mmol), HATU (30 mg, 0.08 mmol) in 10 mL of THE was stirred at r.t. for 4 h. The reaction mixture was diluted with 100 mL of EA, and then washed with H₂O (10 mL×2) and brine (10 mL×2), and the EA layer was dried and concentrated. The crude was purified by reverse phase system to give Intermediate 166 (103 mg). MS: 1224.9 [M+2H]²⁺.

Compound of Example 42. A mixture of Intermediate 166 (103 mg, 0.04 mmol) in DBU/NMP (0.05 mL/10 mL) was stirred at r.t. for 20 min. The mixture was purified by prep-HPLC to afford the compound of Example 42 (40 mg). MS: 1558.6 [M+H]⁺.

### Example 43

### Synthesis of the Compound of Example 43

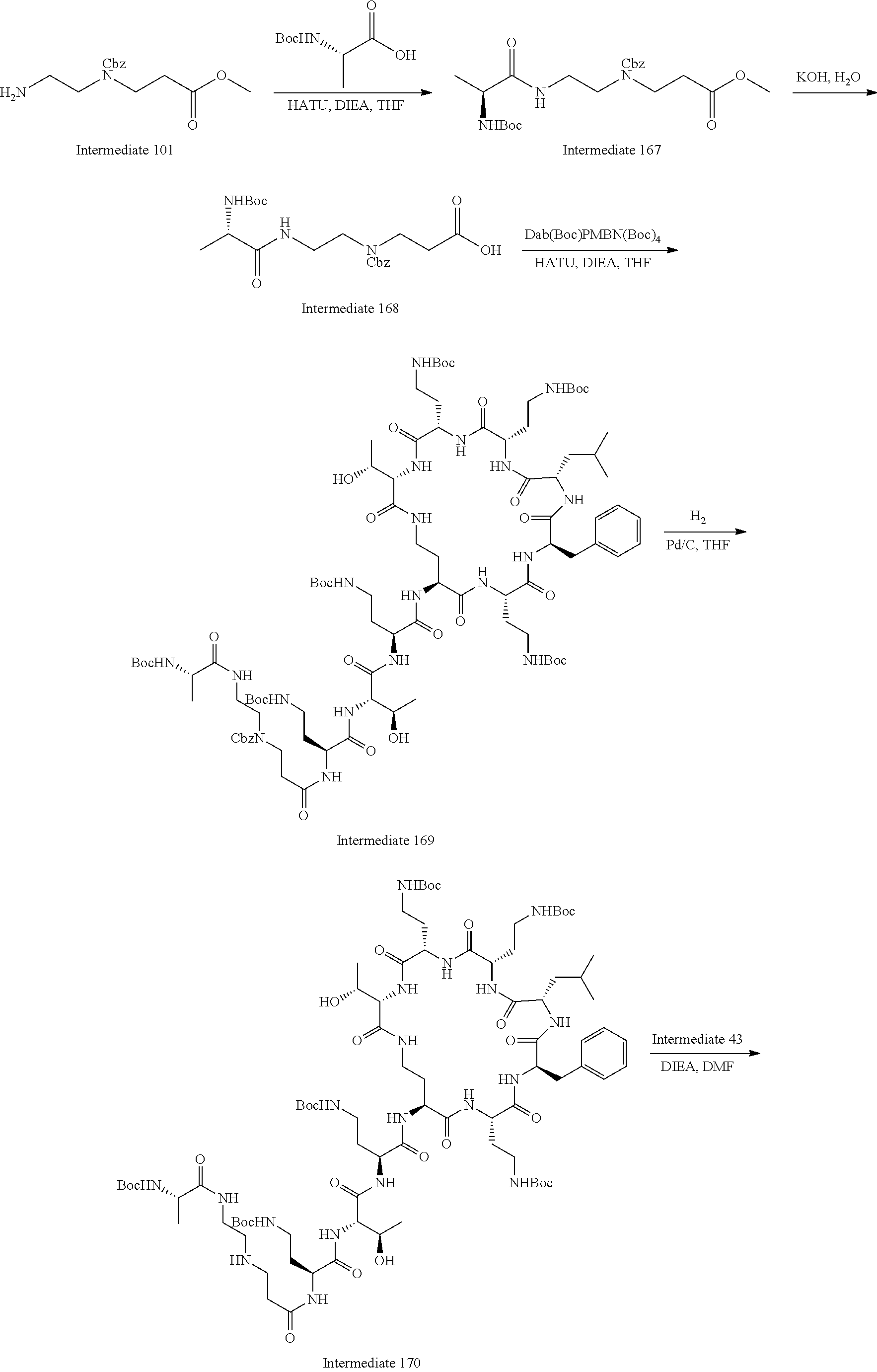

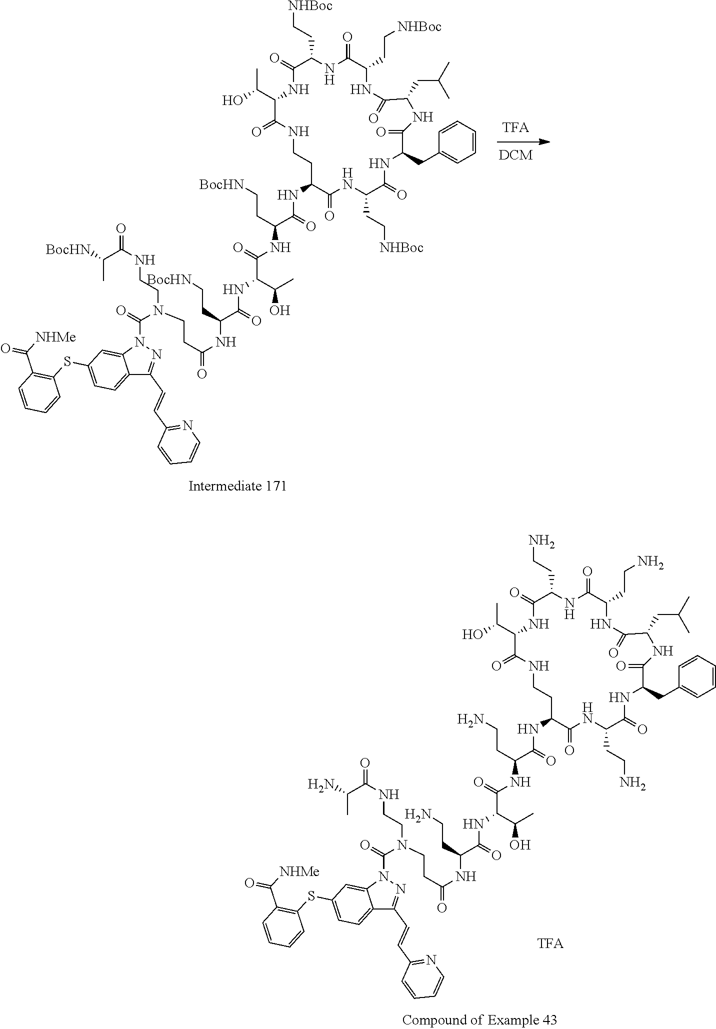

Intermediate 167. Intermediate 101 (650 mg, 2.3 mmol), (S)-2-((tert-butoxycarbonyl)amino)propanoic acid (438 mg, 2.3 mmol), DIEA (848 μL, 4.6 mmol), HATU (1.3 g, 3.5 mmol) in 10 mL of THF were stirred at r.t. for 2 h. The reaction mixture was diluted with 100 mL of EA, and then washed with H₂O (20 mL×2) and brine (20 mL×2), and the EA layer was dried and concentrated. The crude was purified by silica gel column to afford Intermediate 167 (621 mg). MS: 452.0 [M+H]⁺.

Intermediate 168 Intermediate 167 (510 mg, 1.1 mmol), was hydrolyzed with KOH (84 mg, 1.5 mmol) in THF/H₂O (5 mL/5 mL) with string at r.t. for 0.5 h. The mixture was acidified with drops of 0.1M HCl/H₂O to control pH 6, and extracted with EA, washed with H₂O (20 mL×2) and brine (20 mL×2); and the EA layer was dried and concentrated to give Intermediate 168 (436 mg). MS: 438.0 [M+H]⁺.

Intermediate 169. A mixture of Dab(Boc)-PMBN(Boc)₄ (550 mg, 0.35 mmol), Intermediate 168 (231 mg, 0.53 mmol), DIEA (129 μL, 0.7 mmol), HATU (239 mg, 0.63 mmol) in 25 mL of THE was stirred at r.t. for 5 h. The reaction mixture was diluted with 200 mL of EA, and then washed with H₂O (20 mL×2) and brine (10 mL×2), and the EA layer was dried and concentrated. The crude was purified by reverse phase system to give Intermediate 169 (522 mg). MS: 1983.6 [M+H]⁺.

Intermediate 170. Intermediate 169 (522 mg, 0.26 mmol), Pd/C (400 mg) in 20 mL of THE was hydrogenated with H₂ at r.t. for 4 h. The mixture was filtered and concentrated to give Intermediate 170 (462 mg). MS: 1849.2 [M+H]⁺. Intermediate 171. The mixture of Intermediate 105 (0.43 g, 0.23 mmol), Intermediate 43 (0.193 g, 0.35 mmol) and DIEA (85 μL, 0.46 mmol) in 10 mL of DMF was stirred at r.t. for 4 h. The reaction mixture was diluted with 200 mL of EA, and then washed with H₂O (10 mL×2) and brine (10 mL×2), and the EA layer was dried and concentrated. The crude was purified by reverse phase system to give Intermediate 171 (311 mg).

Compound of Example 43. Intermediate 171 (0.31 g, 0.14 mmol) in TFA/DCM (1 mL/10 mL) was stirred at r.t. for 2 h. Volatiles were removed, and the residue was purified by C18 chromatography to afford the compound of Example 43 (155 mg) as TFA salt. MS: 1660.8 [M+H]⁺. NMR: 8.59 (d, J 3.2 Hz, 1H), 8.32 (t, J 5.2 Hz, 1H), 8.12 (d, J 5.6 Hz, 1H), 7.99 (d, J 5.6 Hz, 1H), 7.89 (d, J 11.2 Hz, 1H), 7.73-7.14 (m, 13H), 4.50-4.39 (m, 4H), 4.26-4.06 (m, 8H), 3.90-3.55 (m, 6H), 3.42-3.39 (m, 1H), 3.23-2.91 (m, 12H), 2.81-2.59 (m, 7H), 2.15-1.74 (m, 12H), 1.43-1.04 (m, 11H), 0.75-0.60 (m, 7H).

Optionally, the TFA salt of the compound of Example 43 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chemical Education. 2005, vol. 82, p. 575); or by dissolution of the TFA salt in aq. media, addition of an excess of aq. HCl, followed by lyophilization or direct evaporation of a solution under vacuum. The resulting solid is optionally recrystallized, for example from an alcohol-containing media, such as EtOH-EtOAc, or isopropanol-water, or similar solvent system.

### Example 44

### Synthesis of the Compound of Example 44

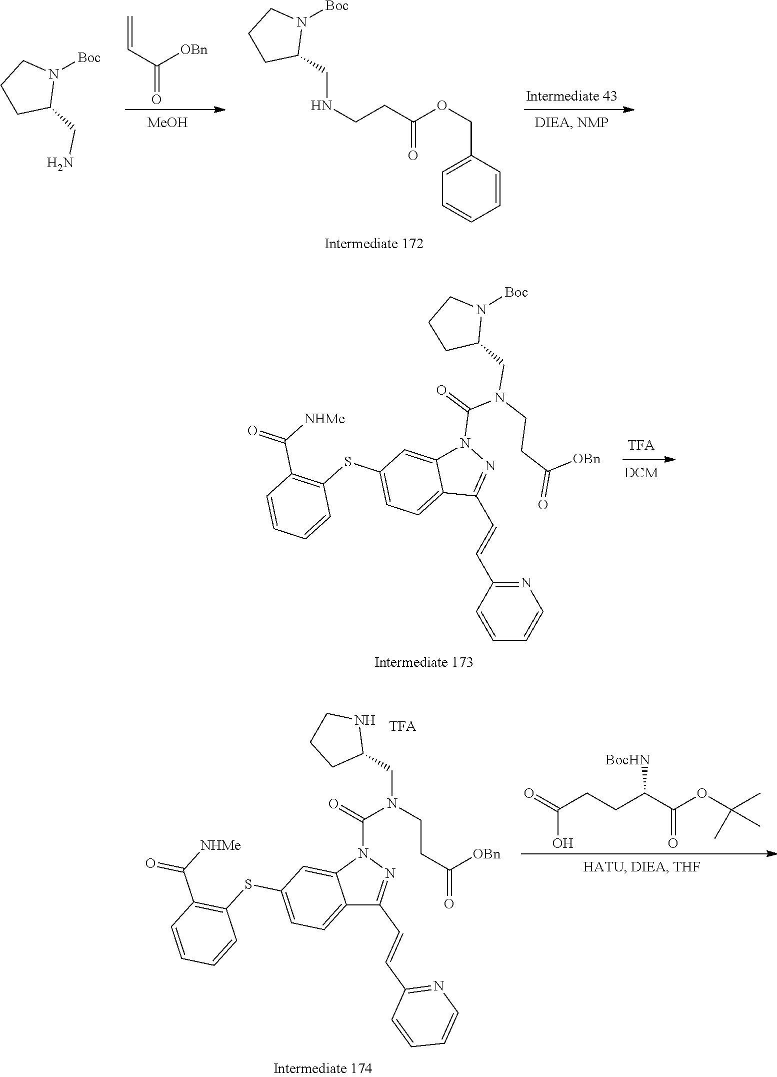

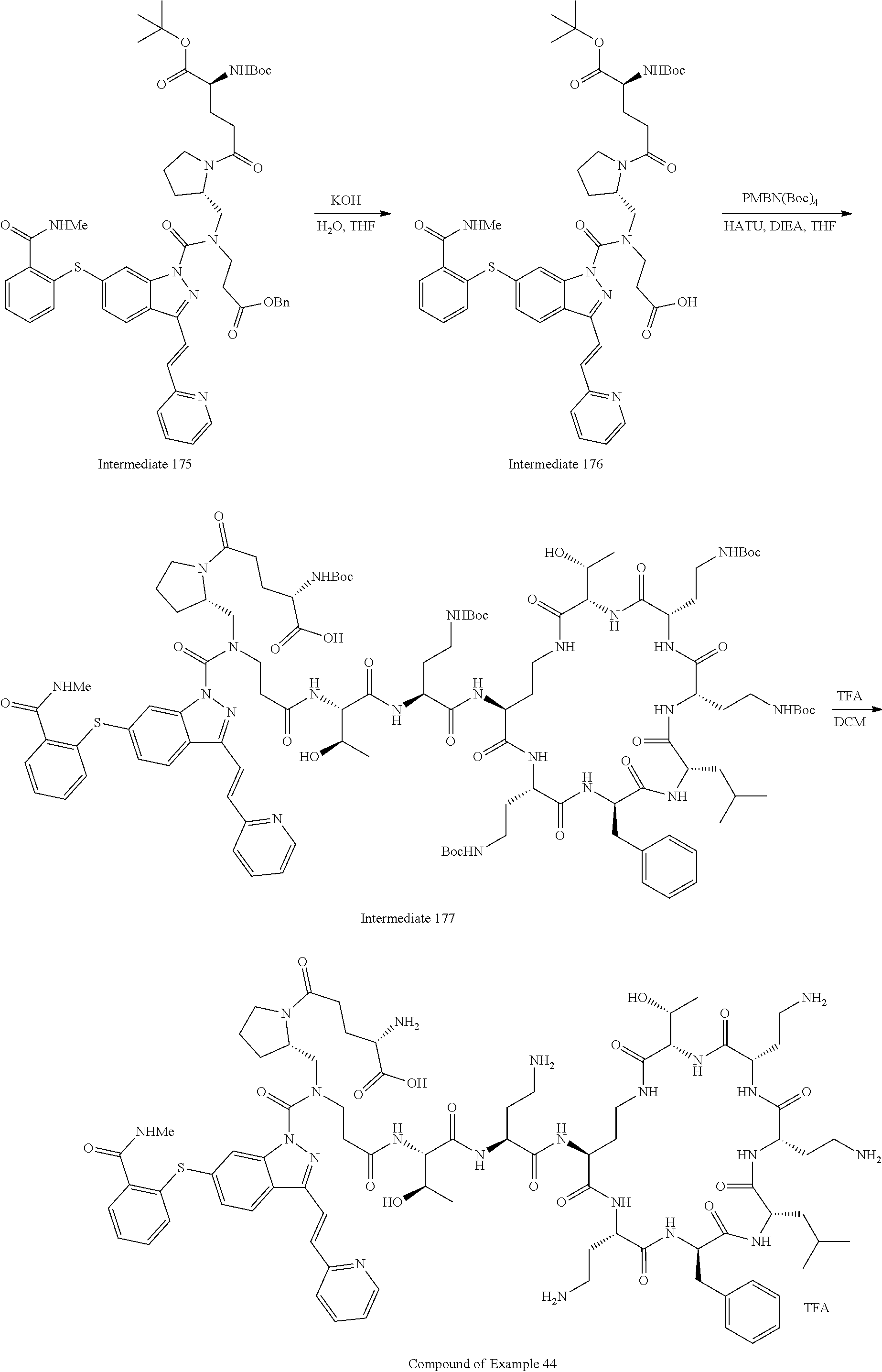

Intermediate 172. The reaction of (S)-tert-butyl 2-(aminomethyl)pyrrolidine-1-carboxylate (2.5 g, 12.5 mmol), benzyl acrylate (2.0 g, 12.5 mmol) in 50 mL of MeOH was stirred at r.t. for 5 h, and concentrated. The product was purified by silica gel column to give Intermediate 172 (855 mg). MS: 363.2 [M+H]⁺.

Intermediate 173 A mixture of Intermediate 172 (186 mg, 0.65 mmol), Intermediate 43 (358 mg, 0.65 mmol), DIEA (240 μL, 1.3 mmol) in 10 mL of NMP was stirred at r.t. for 6 h. The reaction mixture was diluted with 200 mL of EA, and then washed with H₂O (20 mL×2) and brine (10 mL×2), and the EA layer was dried and concentrated. The crude was purified by reverse phase system to afford Intermediate 173 (315 mg). MS: 775.1 [M+H]⁺.

Intermediate 174. A solution of Intermediate 173 (216 mg, 0.3 mmol) in TFA/DCM (2 mL/10 mL) was stirred at r.t. for 4 h. Volatiles were removed under vacuum, and the crude product was used directly at the next step. MS: 675.1 [M+H]⁺.

Intermediate 175. Intermediate 174 (202 mg, 0.3 mmol), (S)-5-(tert-butoxy)-4-((ter.t-butoxycarbonyl)amino)-5-oxopentanoic acid (90 mg, 0.30 mmol), DIEA (112 μL, 0.6 mmol), and HATU (136 mg, 0.36 mmol) in 10 mL of THF were stirred at r.t. for 4 h. The reaction mixture was diluted with 200 mL of EA, and then washed with H₂O (30 mL×2) and brine (20 mL×2), and the EA layer was dried and concentrated. The crude was purified by reverse phase system to give Intermediate 175 (165 mg). MS: 960.1 [M+H]⁺.

Intermediate 176. A mixture of Intermediate 175 (165 mg, 0.2 mmol), KOH (12.8 mg, 0.3 mmol) in 5 mL/5 mL of THF/H₂O was stirred at r.t. for 2 h. 0.1 M HCl aq. was added to control pH=6, then the mixture was extracted with EA (50 mL×3). The EA layer was washed with H₂O (20 mL×2) and brine (20 mL×2), and the EA layer was dried and concentrated. The crude was purified by reverse phase system to give Intermediate 176 (68 mg). MS: 870.1 [M+H]⁺.

Intermediate 177. A mixture of PMBN(Boc)₄ (101 mg, 0.07 mmol), Intermediate 176 (48 mg, 0.07 mmol), DIEA (26 μL, 0.14 mmol), and HATU (38 mg, 0.1 mmol) in 5 mL of THF was stirred at r.t. for 6 h. The reaction mixture was diluted with 50 mL of EA, and then washed with H₂O (10 mL×2) and brine (10 mL×2). The EA layer was dried (Na₂SO₄) and concentrated under vacuum. The crude material was purified by C18 column chromatography to give Intermediate 177 (66 mg).

Compound of Example 44. Intermediate 177 (60 mg, 0.02 mmol) in TFA/DCM (0.5 mL/5 mL) was stirred at r.t. for 2 h. Volatiles were removed, and the residue was purified by C18 column chromatography (ACN/H₂O=0˜40%) to the compound of Example 44 as TFA salt (25 mg). MS: 1659.6 [M+H]⁺. NMR: 8.56 (d, J 4.0 Hz, 1H), 8.29-8.27 (m, 1H), 7.98-7.63 (m, 4H), 7.54-7.08 (m, 13H), 4.47-4.29 (m, 3H), 4.18-4.06 (m, 9H), 3.71-3.51 (m, 3H), 3.21-3.16 (m, 1H), 3.00-2.46 (m, 19H), 2.17-1.62 (m, 17H), 1.38-1.21 (m, 3H), 1.05-0.91 (m, 6H), 0.70-0.52 (m, 8H).

### Example 45

### Synthesis of the Compound of Example 45

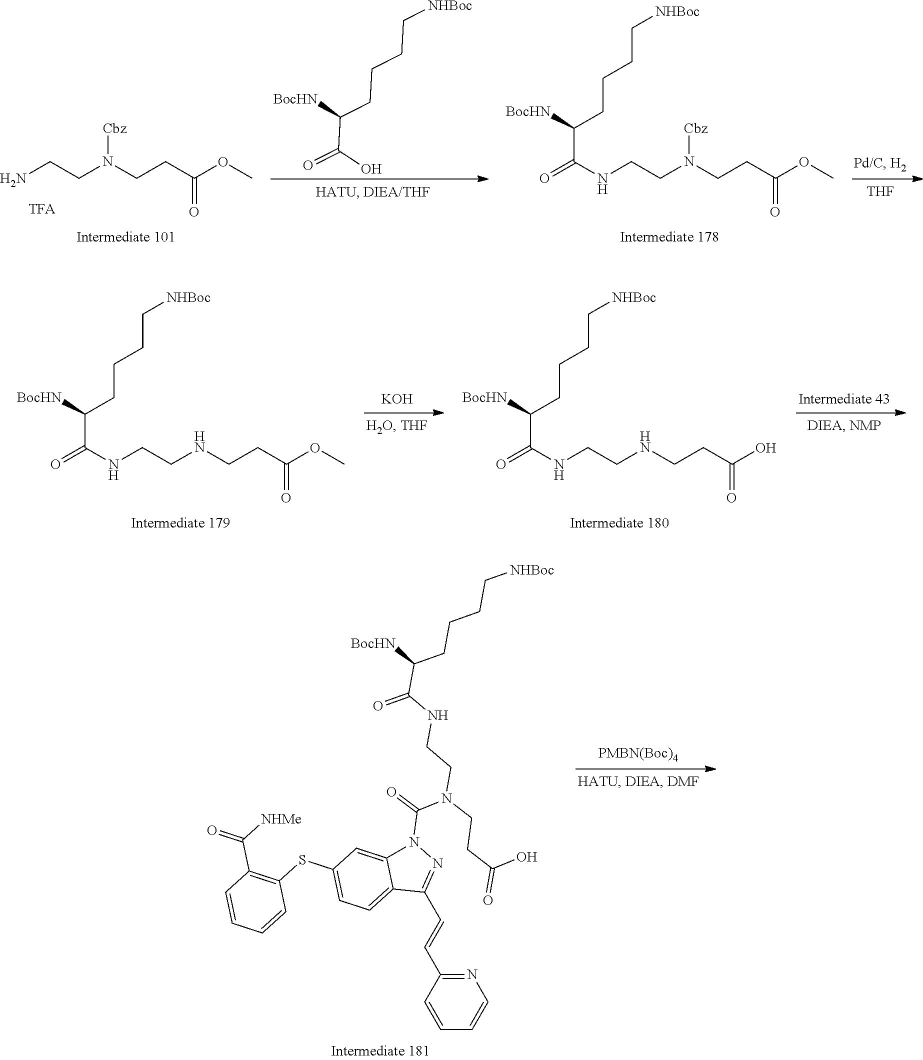

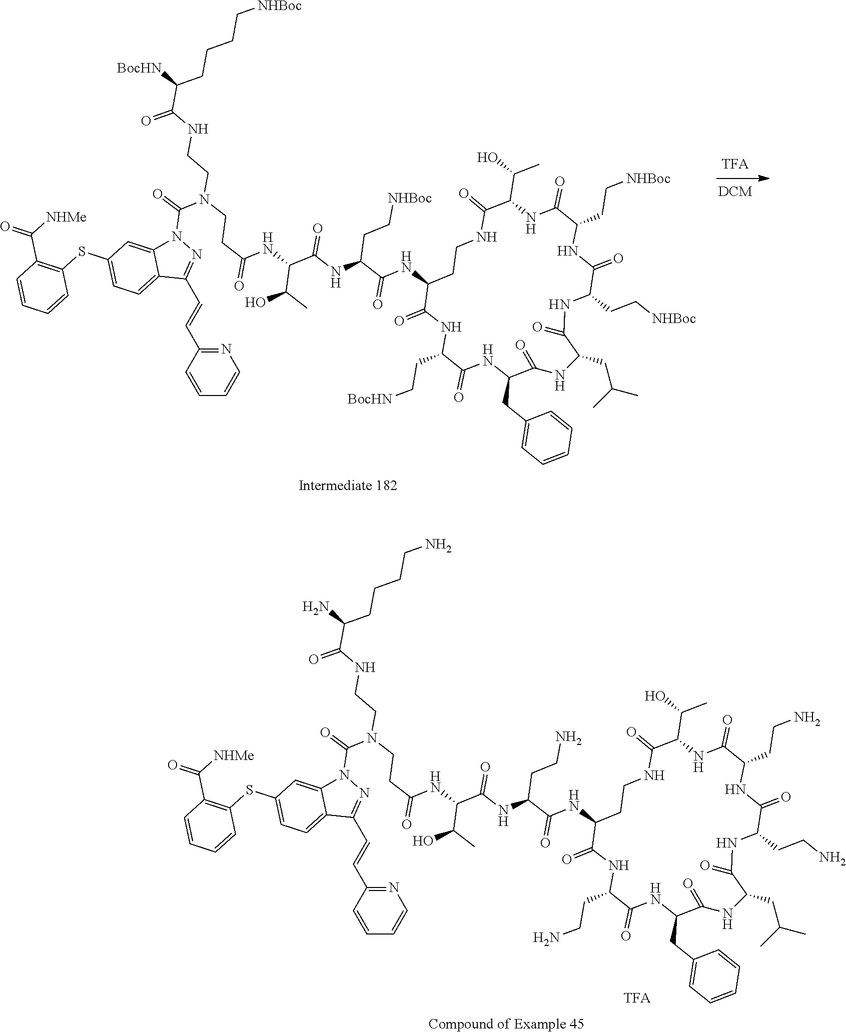

Intermediate 178. The mixture of Intermediate 101 (500 mg, 1.8 mmol), (S)-2,6-bis((tert-butoxycarbonyl)amino)hexanoic acid (619 mg, 1.8 mmol), HATU (1.0 g, 2.7 mmol) and DIEA (0.66 mL, 3.6 mmol) in THE (50 mL) was stirred at r.t. for 5 h. The mixture was cooled to r.t., and extracted with EA (200 mL). EA layer was washed with H₂O (25 mL×2) and brine (25 mL), dried (Na₂SO₄) and evaporated under vacuum. The residue was purified by silica gel chromatography to give Intermediate 178 (588 mg). MS: 609.1 [M+H]⁺.

Intermediate 179. A mixture os Intermediate 178 (588 mg, 0.9 mmol) and Pd/C (0.5 g, 56% H₂O) in THF (25 mL) was stirred at r.t. under H₂ for 12 h. The mixture was filtered and the filtrate was evaporated under vacuum to give Intermediate 179 (545 mg). MS: 475.2 [M+H]⁺.

Intermediate 180. To a solution of KOH (36 mg, 0.6 mmol) in THF/H₂O (10 mL/10 mL) was added Intermediate 179 (200 mg, 0.4 mmol) and the reaction was stirred at r.t. for 2 h. 0.1 M HCl was added to pH of about 5.0. The mixture was then extracted with EA (100 mL), washed with H₂O (15 mL×2) and brine (15 mL). The EA layer was dried (Na₂SO₄) and evaporated under vacuum. The residue was purified by C18 chromatography to give Intermediate 180 (156 mg). MS: 461.2 [M+H]⁺.

Intermediate 181. The mixture of Intermediate 43 (280 mg, 0.5 mmol), Intermediate 180 (209 mg, 0.5 mmol) and DIEA (0.184 mL, 1.0 mmol) in NMP (10 mL) was stirred at r.t. under Ar for 16 h. The product was extracted with EA (150 mL), washed with brine(15 mL×3), dried and evaporated. The residue was purified by C18 column chromatography (ACN/H₂O=0˜90%) to give Intermediate 181 (356 mg). MS: 873.2 [M+H]⁺.

Intermediate 182. Intermediate 181 (262 mg, 0.3 mmol), Dab(Boc)-PMBN(Boc)₄ (400 mg, 0.3 mmol), HATU (171 mg, 0.45 mmol) and DIEA (0.11 mL, 0.6 mmol) in THE (15 mL) were mixed together, and the reaction was stirred at 25° C. under Ar for 6 h. Extracted with EA (250 mL), washed with H₂O (25 mL×2) and brine (25 mL). The EA layer was dried and evaporated. The residue was purified by C18 column chromatography (ACN/H₂O 0˜90%) to give Intermediate 182 (368 mg).

Compound of Example 45. Intermediate 182 (260 mg, 0.1 mmol) in TFA/DCM (1 mL/3 mL) was stirred at r.t. for 2 h. Volatiles were removed, and the residue was purified by C18 column chromatography (ACN/H₂O=0˜50%) to afford the compound of Example 45 as a TFA salt (134 mg). MS: 1617.7 [M+H]⁺. NMR: 8.61 (d, J 5.6 Hz, 1H), 8.31 (t, J 6.4 Hz, 1H), 8.13-7.89 (m, 2H), 7.73-7.69 (m, 2H), 7.53-7.44 (m, 4H), 7.32-7.17 (m, 6H), 4.59-4.37 (m, 4H), 4.22-3.61 (m, 14H), 3.49-3.46 (m, 1H), 3.24-3.21 (m, 1H), 3.06-3.80 (m, 8H), 2.80-2.61 (m, 9H), 2.21-1.68 (m, 12H), 1.43-1.01 (m, 12H), 0.77-0.62 (m, 7H).

Optionally, the TFA salt of the compound of Example 45 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chemical Education. 2005, vol. 82, p. 575); or by dissolution of the TFA salt in aq. media, addition of an excess of aq. HCl, followed by lyophilization or direct evaporation of a solution under vacuum. The resulting solid is optionally recrystallized, for example from an alcohol-containing media, such as EtOH-EtOAc, or isopropanol-water, or similar solvent system.

### Example 46

### Synthesis of the Compound of Example 46

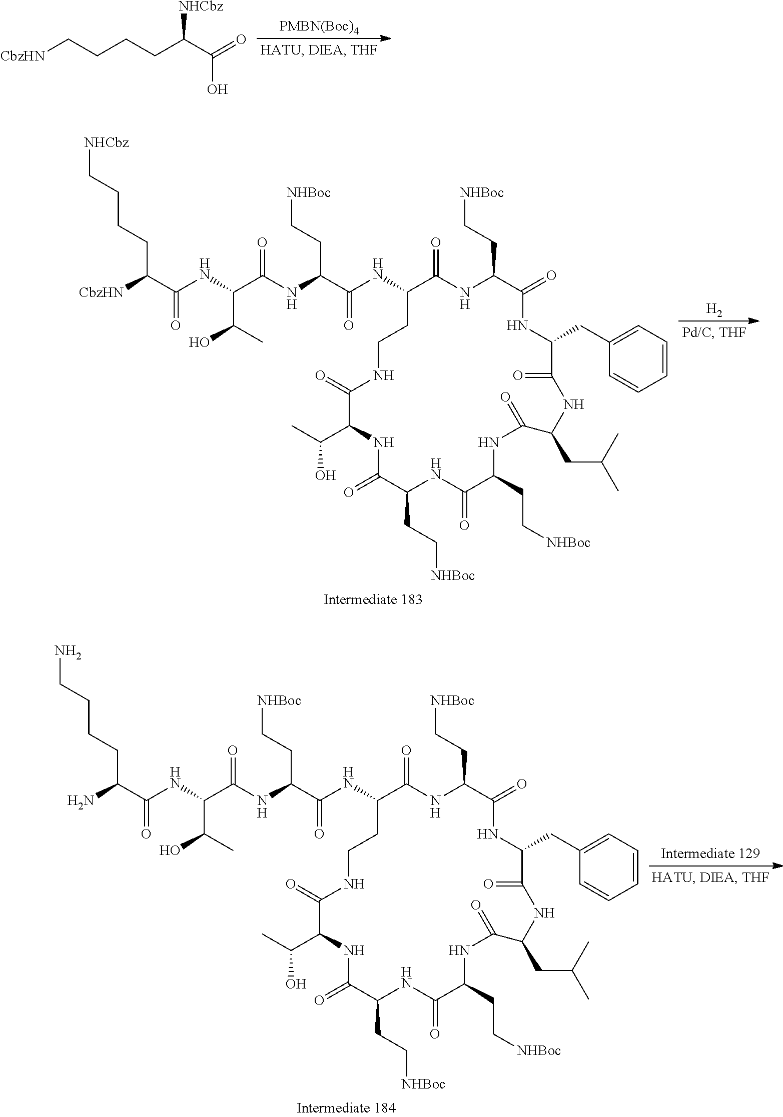

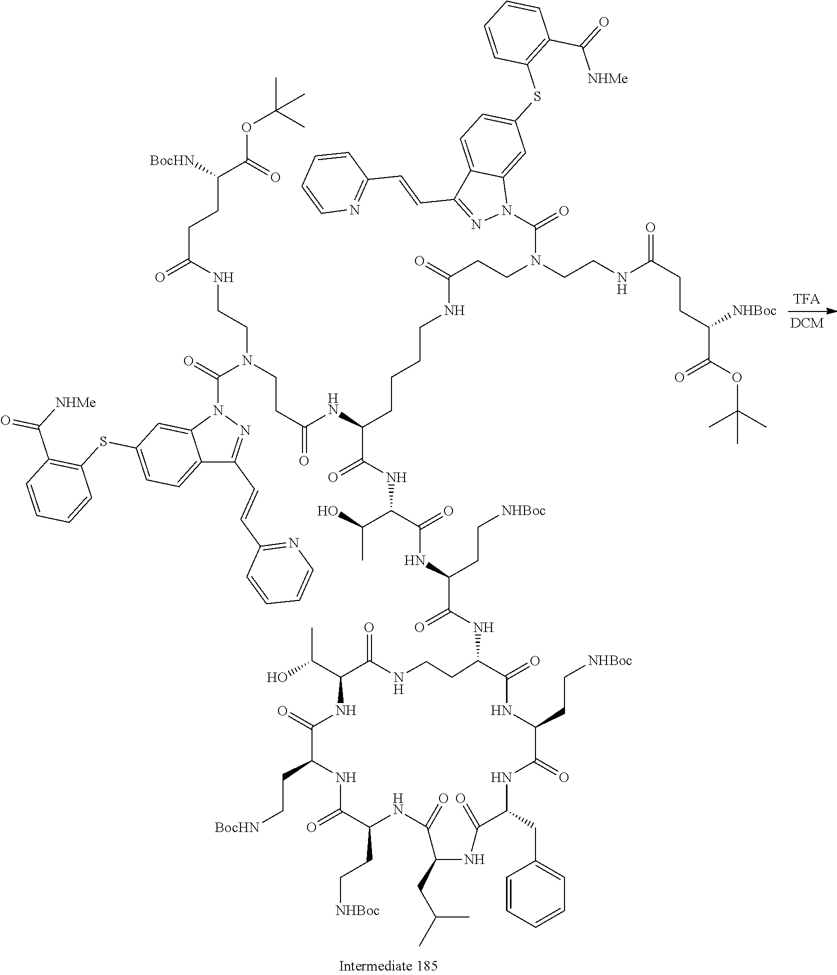 [SMILES: CNC(=O)c1ccccc1Sc1ccc2c(/C=C/c3ccccn3)nn(C(=O)N(CCNC(=O)CC[C@H](NC(=O)OC(C)(C)C)C(=O)OC(C)(C)C)CCC(=O)NCCCC[C@H](NC(=O)CCN(CCNC(=O)CC[C@H](NC(=O)OC(C)(C)C)C(=O)OC(C)(C)C)C(=O)n3nc(/C=C/c4ccccn4)c4ccc(Sc5ccccc5C(=O)NC)cc43)C(=O)N[C@H](C(=O)N[C@@H](CCNC(=O)OC(C)(C)C)C(=O)N[C@H]3CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc4ccccc4)NC(=O)[C@H](CCNC(=O)OC(C)(C)C)NC3=O)[C@@H](C)O)c2c1]

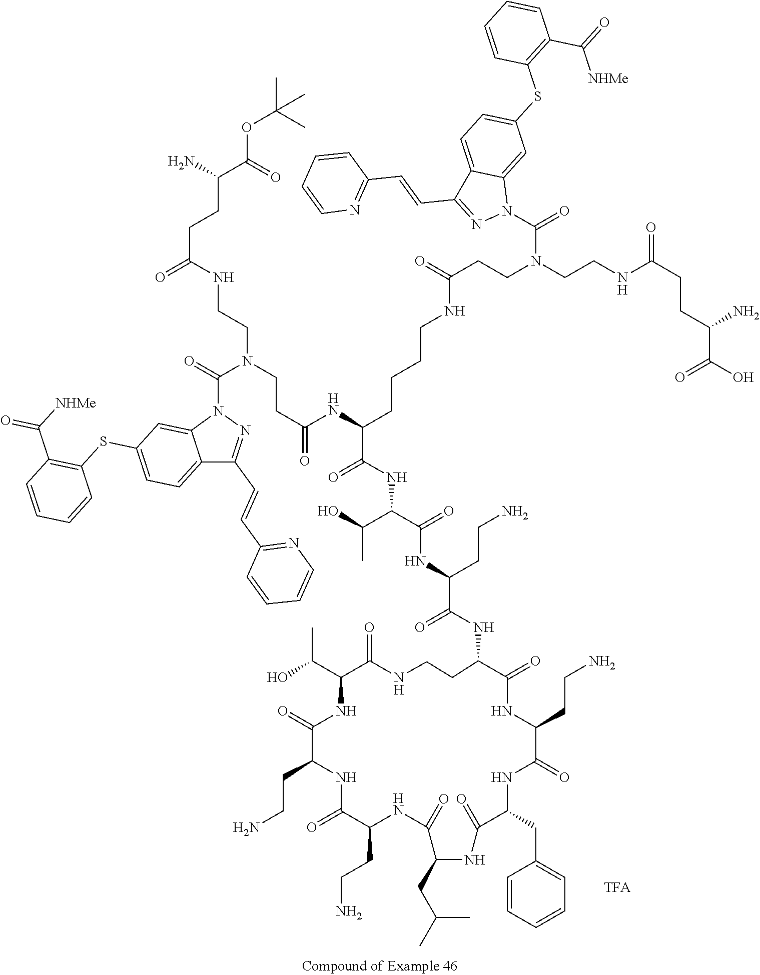 [SMILES: CNC(=O)c1ccccc1Sc1ccc2c(/C=C/c3ccccn3)nn(C(=O)N(CCNC(=O)CC[C@H](N)C(=O)O)CCC(=O)NCCCC[C@H](NC(=O)CCN(CCNC(=O)CC[C@H](N)C(=O)OC(C)(C)C)C(=O)n3nc(/C=C/c4ccccn4)c4ccc(Sc5ccccc5C(=O)NC)cc43)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Intermediate 183. PMBN(Boc)₄ (500 mg, 0.37 mmol), (S)-2,6-bis(((benzyloxy)carbonyl)amino)hexanoic acid (442 mg, 0.8 mmol), HATU (380 mg, 1.0 mmol) and DIEA (0.37 mL, 2.0 mmol) was mixed together in THE (15 mL). The reaction was stirred at r.t. for 6 h. The mixture was concentrated, diluted with EA (200 mL), washed with H₂O (25 mL×2) and brine (25 mL). The EA layer was dried and evaporated. The residue was purified by silica gel chromatography to give Intermediate 183 (488 mg). MS: 1760.3 [M+H]⁺.

Intermediate 184 Intermediate 183 (380 mg, 0.22 mmol) and Pd/C (0.5 g, 56% H₂O) in THE (25 mL) was degassed with H₂, then stirred under H₂ at r.t. for 12 h. The mixture was filtered and dried to give crude Intermediate 184, and used directly for next step.

Intermediate 185. Intermediate 184 (270 mg, 0.18 mmol), Intermediate 129 (332 mg, 0.40 mmol) HATU (190 mg, 0.50 mmol) and DIEA (0.19 mL, 1.0 mmol) in THE (15 mL) were stirred at r.t. for 10 h. the mixture was concentrated, and the crude was dissolved with EA (150 mL), washed with H₂O (15 mL×2) and brine (15 mL). The EA layer was dried (Na₂SO₄) and evaporated under vacuum. Purification by reverse phase system gave Intermediate 185 (311 mg). MS: 1558.1 [M+2H]²⁺.

Compound of Example 46. Intermediate 185 (300 mg, 0.1 mmol) in TFA/DCM (2 mL/6 mL) was stirred at r.t. for 12 h. Volatiles were removed under vacuum and the residue was purified by C18 column chromatography (ACN/H₂O 0˜50%) to the compound of Example 46 as TFA salt (62 mg). MS: 1202.4 [M+2H]²⁺ NMR: 8.62-8.36 (m, 4H), 8.18-7.93 (m, 3H), 7.84-6.99 (m, 23H), 4.57-4.08 (m, 12H), 3.74-3.18 (m, 15H), 3.01-2.89 (m, 9H), 2.75-2.42 (m, 12H), 2.20-1.70 (m, 17H), 1.38-1.01 (m, 12H), 0.72-0.55 (m, 7H).

### Example 47

### Synthesis of the Compound of Example 47

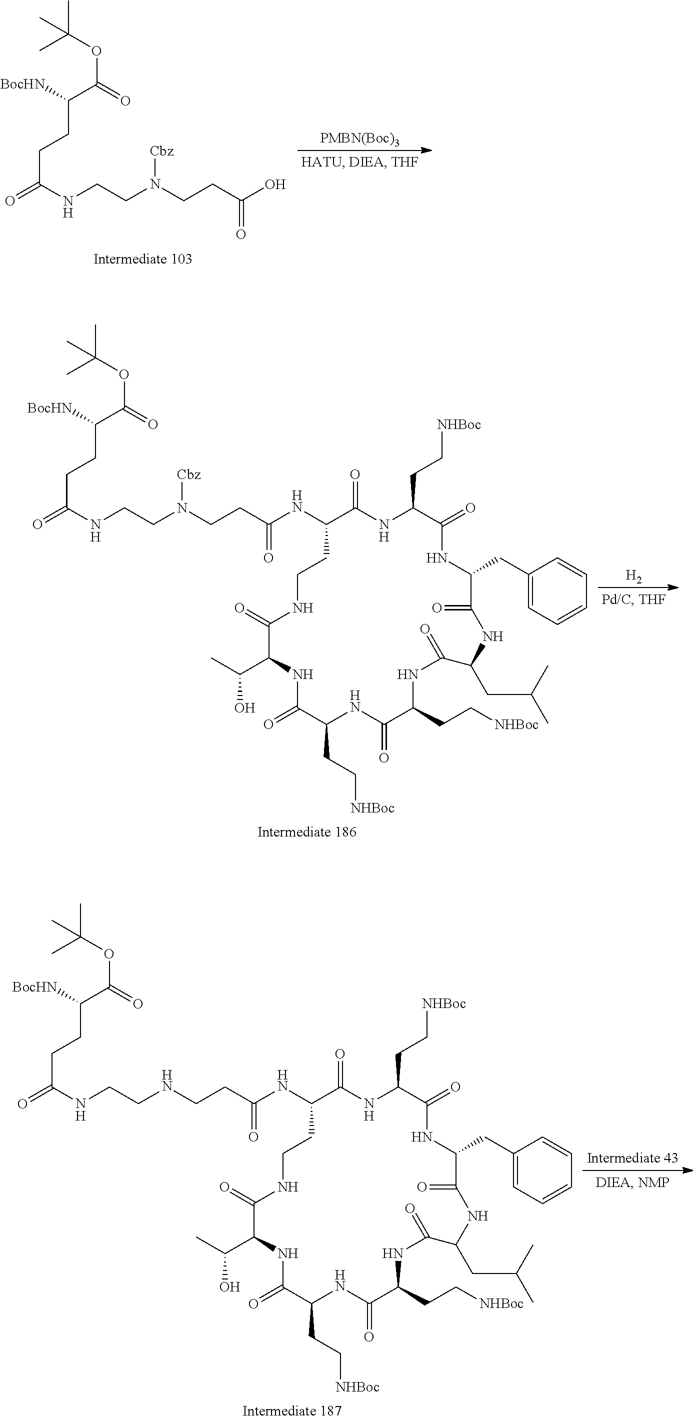

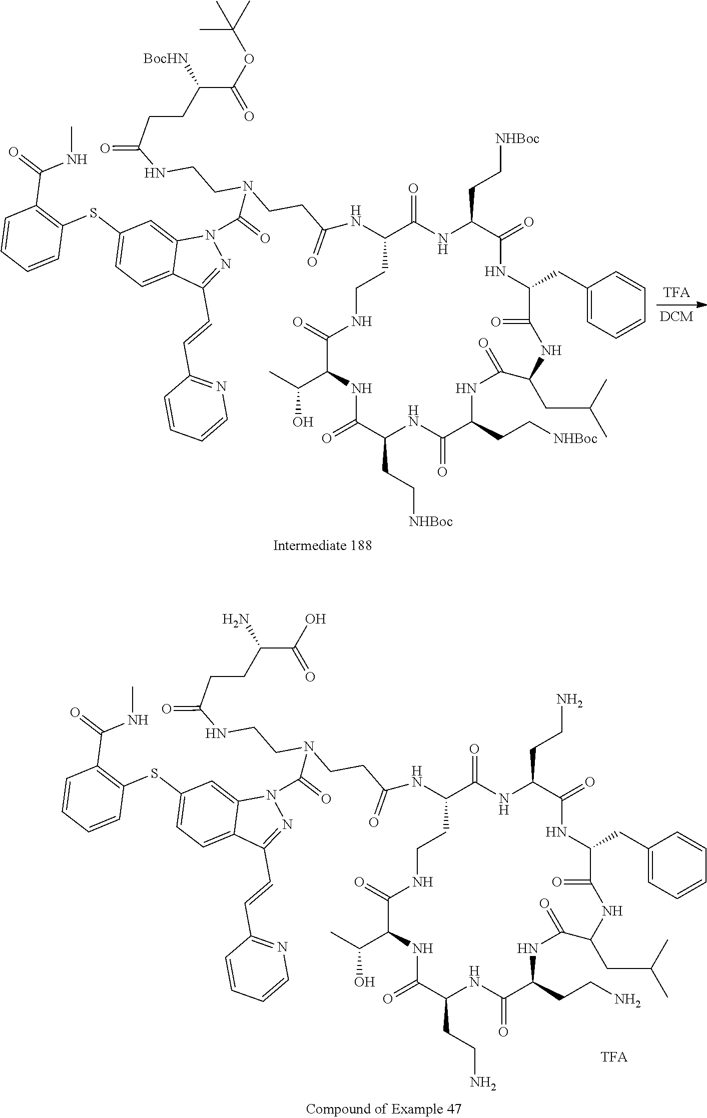 [SMILES: CNC(=O)c1ccccc1Sc1ccc2c(/C=C/c3ccccn3)nn(C(=O)N(CCNC(=O)CC[C@H](NC(=O)OC(C)(C)C)C(=O)OC(C)(C)C)CCC(=O)N[C@H]3CCNC(=O)[C@H]([C@@H](C)O)NC(=O)[C@H](CCNC(=O)OC(C)(C)C)NC(=O)[C@H](CCNC(=O)OC(C)(C)C)NC(=O)[C@H](CC(C)C)NC(=O)[C@@H](Cc4ccccc4)NC(=O)[C@H](CCNC(=O)OC(C)(C)C)NC3=O)c2c1 ; CNC(=O)c1ccccc1Sc1ccc2c(/C=C/c3ccccn3)nn(C(=O)N(CCNC(=O)CC[C@H](N)C(=O)O)CCC(=O)N[C@H]3CCNC(=O)[C@H]([C@@H](C)O)NC(=O)[C@H](CCN)NC(=O)[C@H](CCN)NC(=O)C(CC(C)C)NC(=O)[C@@H](Cc4ccccc4)NC(=O)[C@H](CCN)NC3=O)c2c1]

Intermediate 186. The mixture of cyclo[Dab-Dab(Boc)-D-Phe-Leu-Dab(Boc)-Dab(Boc)-Thr] (same as PMBH(Boc)₃; 300 mg, 0.30 mmol, synthesized according to a procedure by Li et al. in Synthesis. 2015, p. 2088), Intermediate 103 (165 mg, 0.30 mmol), HATU (171 mg, 0.45 mmol) and DIEA (0.11 mL, 0.60 mmol) in THF (25 mL) was stirred at r.t. for 12 h. The mixture was concentrated under vacuum, diluted with EA (200 mL). and washed with H₂O (25 mL×2) and brine (25 mL). The EA layer was dried (Na₂SO₄) and evaporated. The residue was purified by silica gel chromatography to give Intermediate 186 (486 mg). MS: 1595.5 [M+H]⁺.

Intermediate 187 The suspension of Intermediate 186 (380 mg, 0.24 mmol) and 10% Pd/C (0.5 g, contains 56% H₂O) in THF (25 mL) was degassed with H₂, then stirred under H₂ at r.t. for 16 h, filtered and dried to give crude Intermediate 187, which was directly used in next step. MS: 1461.9 [M+H] *.

Intermediate 188. The mixture of Intermediate 187 (311 mg, 0.2 mmol), Intermediate 43 (112 mg, 0.20 mmol) and DIEA (0.074 mL, 0.4 mmol) in DMP (15 mL) was stirred at r.t. for 6 h. Volatiles were evaporated under vacuum, the residue was dissolved with EA (150 mL), washed with H₂O (15 mL×2) and brine (15 mL). The EA layer was dried and evaporated. The residue was purified by reverse phase system to give Intermediate 188 (186 mg). MS: 1872.9 [M+H]⁺.

Compound of Example 47. Intermediate 188 (380 mg, 0.1 mmol) in TFA/DCM (2 mL/10 mL) was stirred at r.t. for 7 h. Volatiles were removed under vacuum, and the residue was purified by C18 column chromatography (ACN/H₂O 0˜50%) to afford the compound of Example 47 as TFA salt (86 mg). MS: 1418.6 [M+H]⁺. NMR: 8.66 (d, J 5.6 Hz, 1H), 8.52 (t, J 7.2 Hz, 1H), 8.31 (d, J 8.4 Hz, 1H), 8.00-7.88 (m, 3H), 7.70-7.64 (m, 2H), 7.50-7.47 (m, 4H), 7.31-7.16 (m, 6H), 4.47-3.73 (m, 14H), 3.37 (s, 2H), 3.21-2.62 (m, 16H), 2.21-1.73 (m, 12H), 1.40 (s, 2H), 1.02-0.64 (m, 11H).

Optionally, the TFA salt of the compound of Example 47 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chemical Education. 2005, vol. 82, p. 575); or by dissolution of the TFA salt in aq. media, addition of an excess of aq. HCl, followed by lyophilization or direct evaporation of a solution under vacuum. The resulting solid is optionally recrystallized, for example from an alcohol-containing media, such as EtOH-EtOAc, or isopropanol-water, or similar solvent system.

### Example 48

### Synthesis of the Compound of Example 48

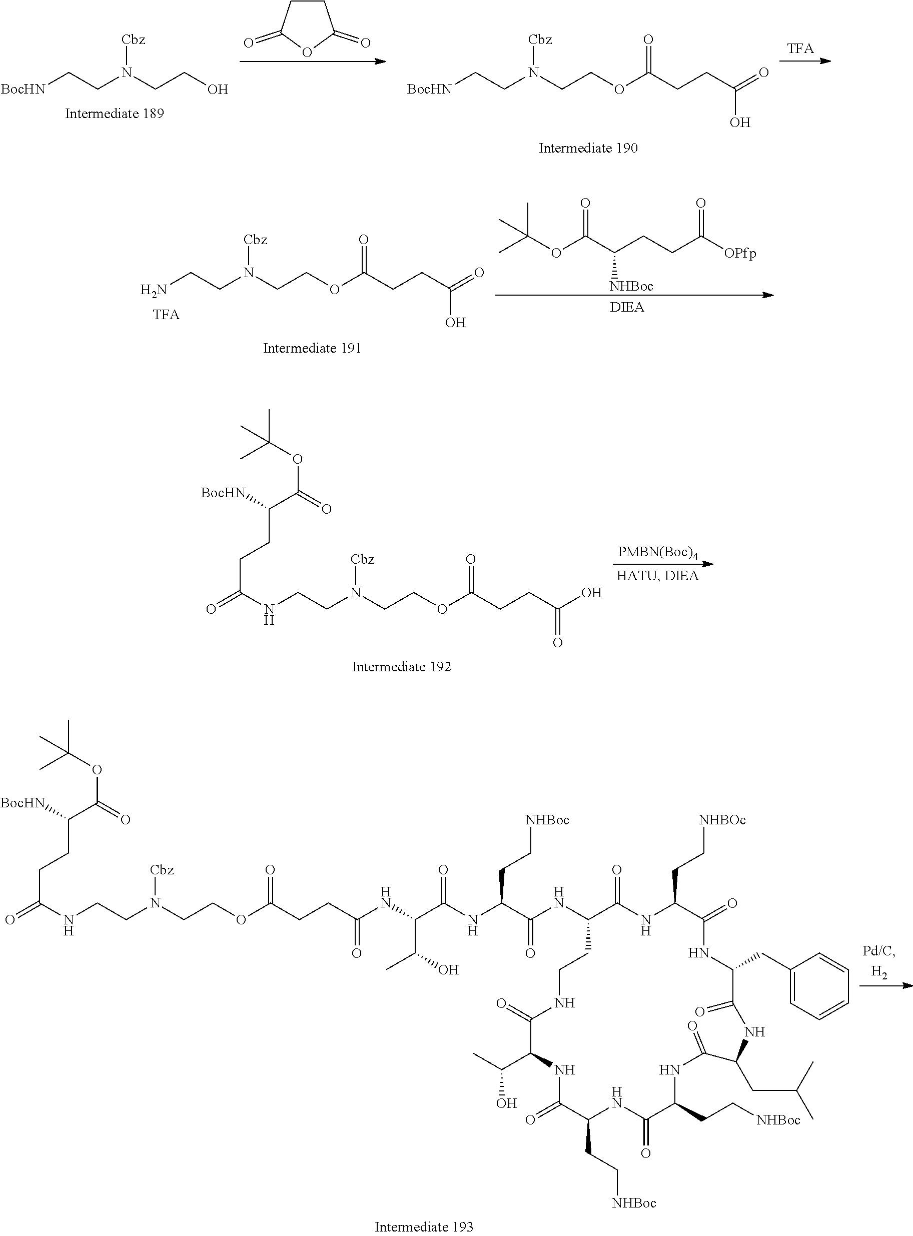

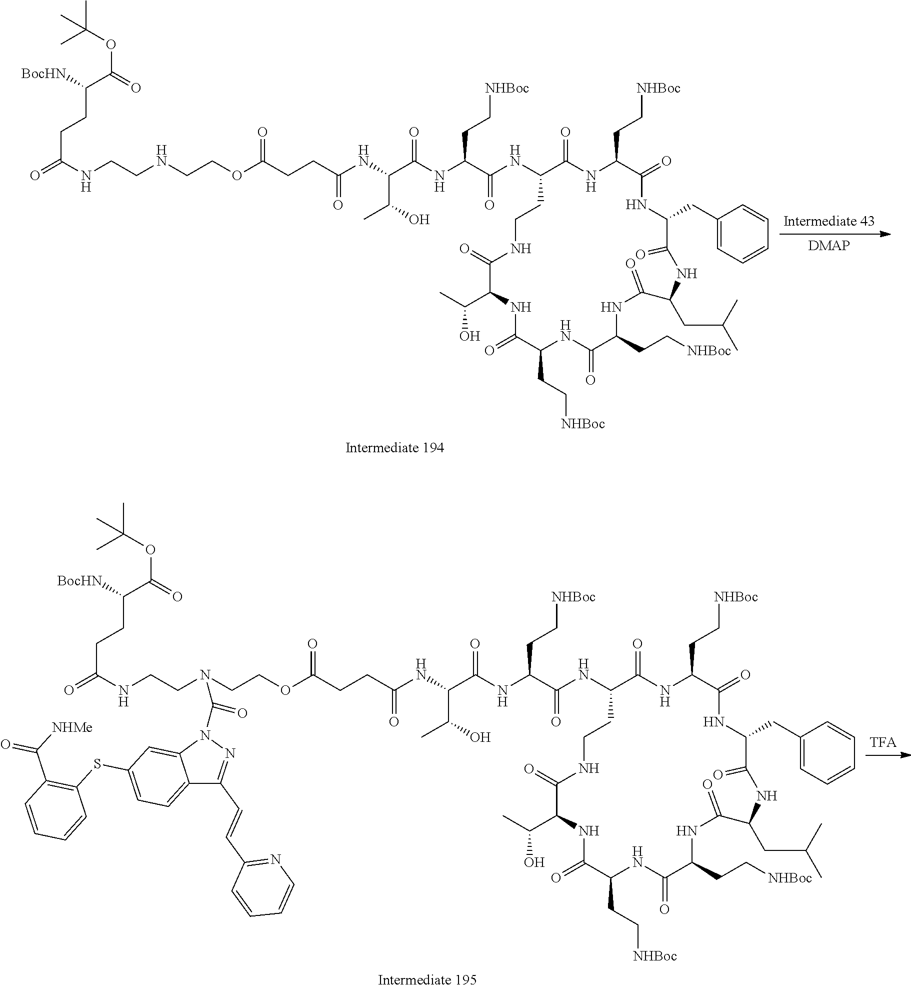

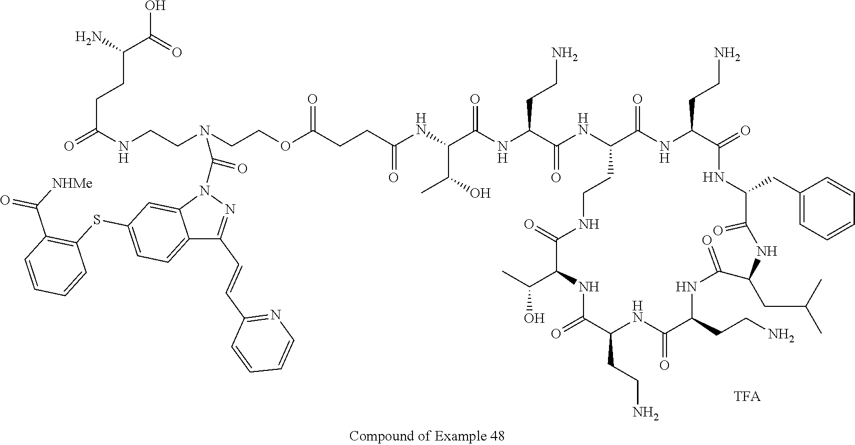 [SMILES: CNC(=O)c1ccccc1Sc1ccc2c(/C=C/c3ccccn3)nn(C(=O)N(CCNC(=O)CC[C@H](N)C(=O)O)CCOC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Compound of Example 48. The compound of Example 48 (TFA salt form) is prepared analogously to the procedures described for the preparation of the compound of Example 29, except using Intermediate 192 instead of the Intermediate 103 at the amide coupling step with PMBN(Boc)₄, and using Intermediate 194 instead of the Intermediate 105 for respective reaction with the Intermediate 43 (which is used in identical manner for the preparations of both the compounds of Examples 29 and 48).

### Example 49

### Synthesis of the Compound of Example 49

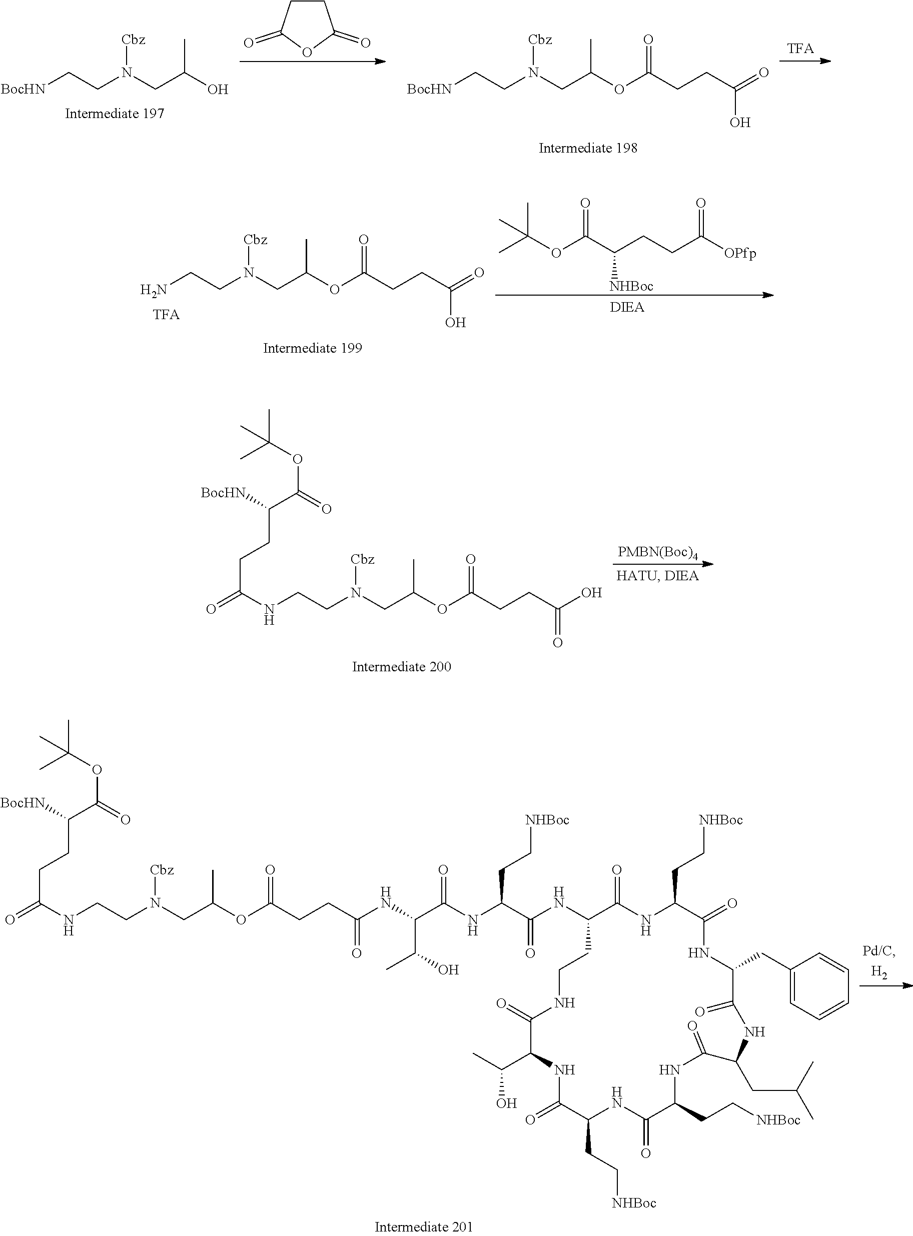

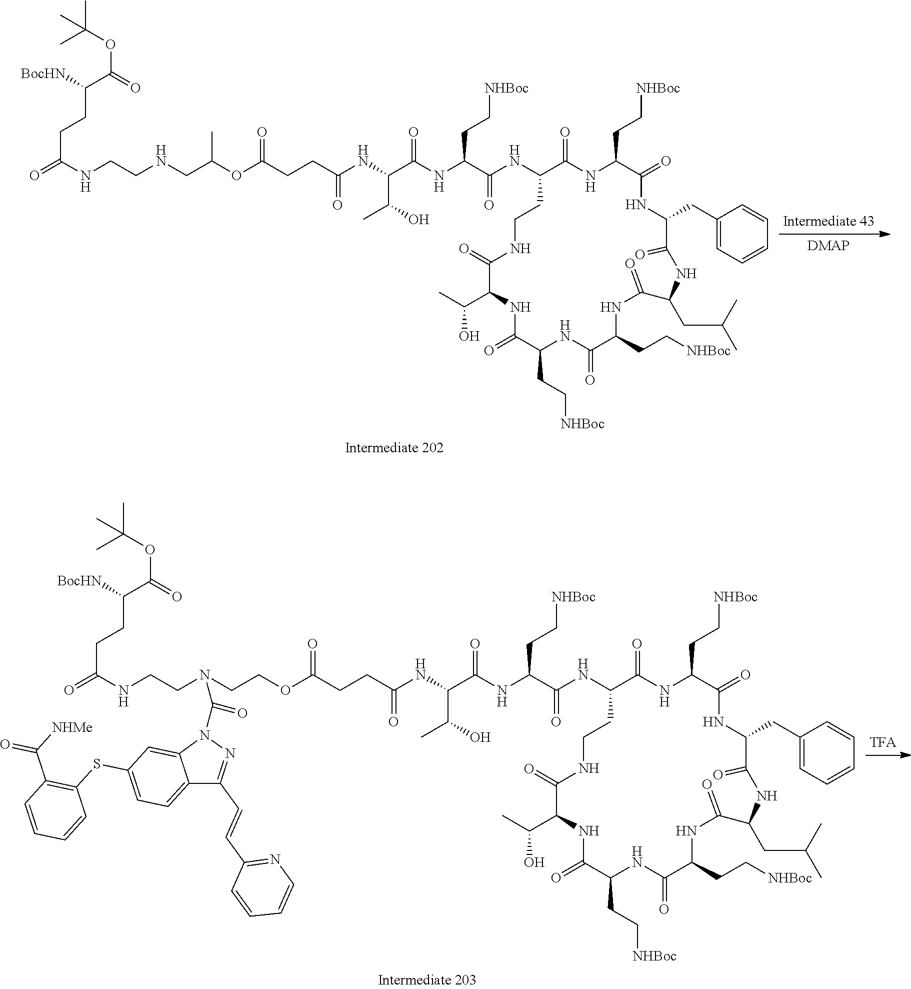

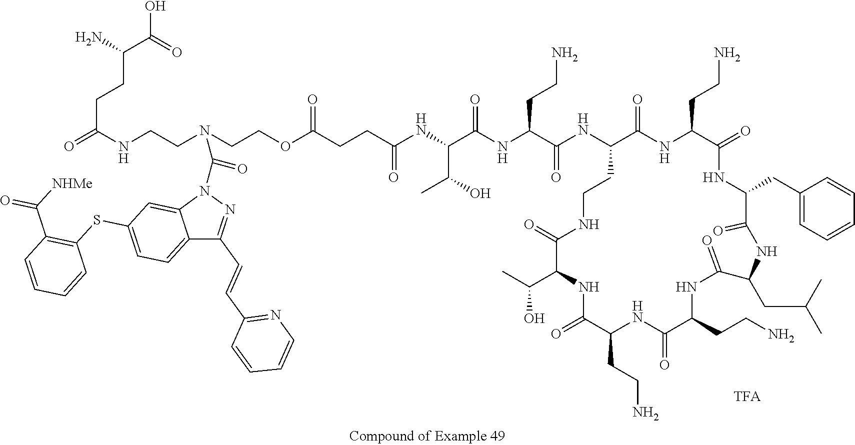 [SMILES: CNC(=O)c1ccccc1Sc1ccc2c(/C=C/c3ccccn3)nn(C(=O)N(CCNC(=O)CC[C@H](N)C(=O)O)CCOC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Compound of Example 49. The compound of Example 49 (TFA salt form) is prepared analogously to the procedures described for the preparation of the compound of Example 29, except using Intermediate 200 instead of the Intermediate 103 at the amide coupling step with PMBN(Boc)₄, and using Intermediate 202 instead of the Intermediate 105 for respective reaction with the Intermediate 43 (which is used in identical manner for the preparation of both the compounds of Examples 29 and 49).

### Example 50

### Synthesis of the Compound of Example 50

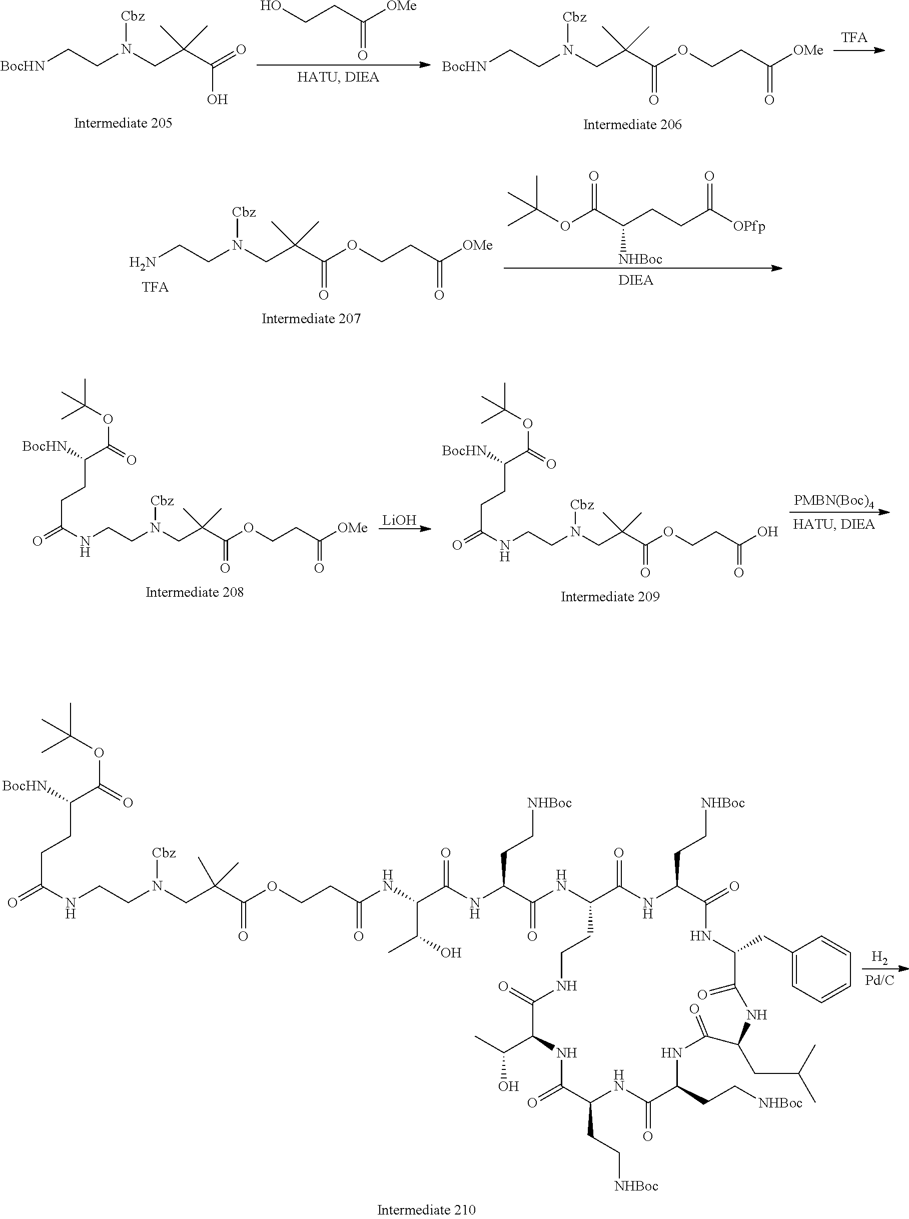

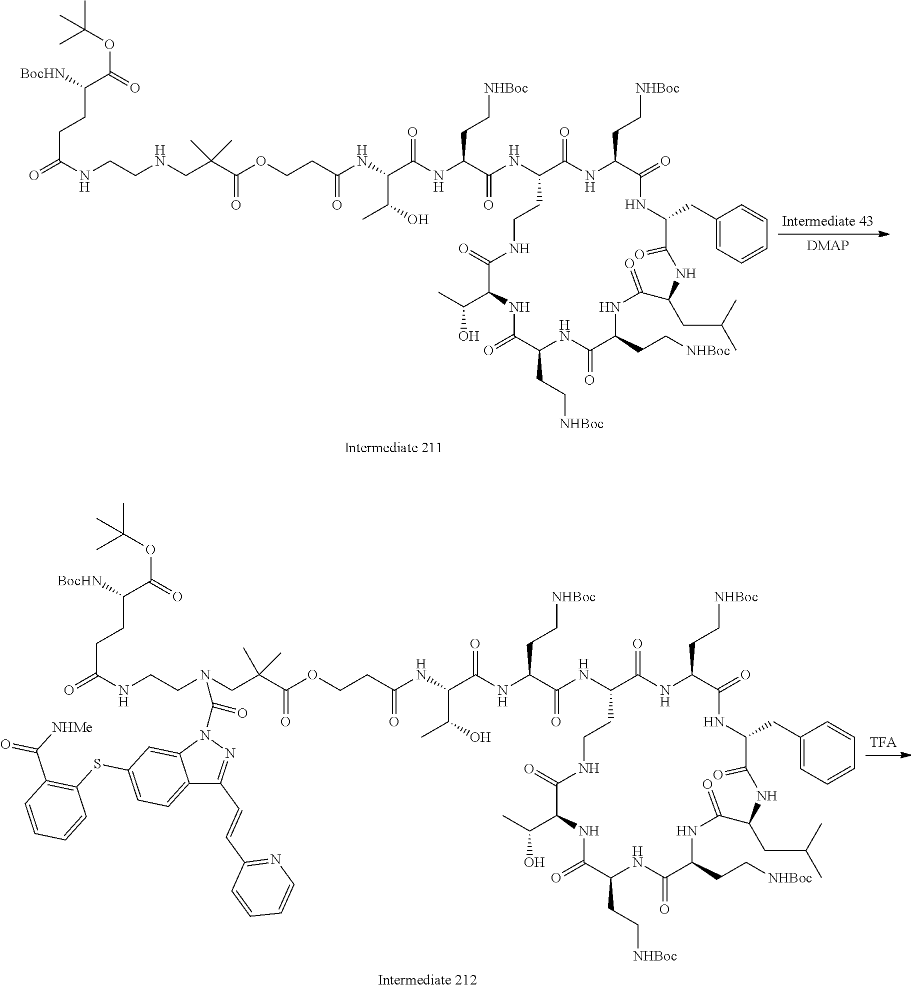

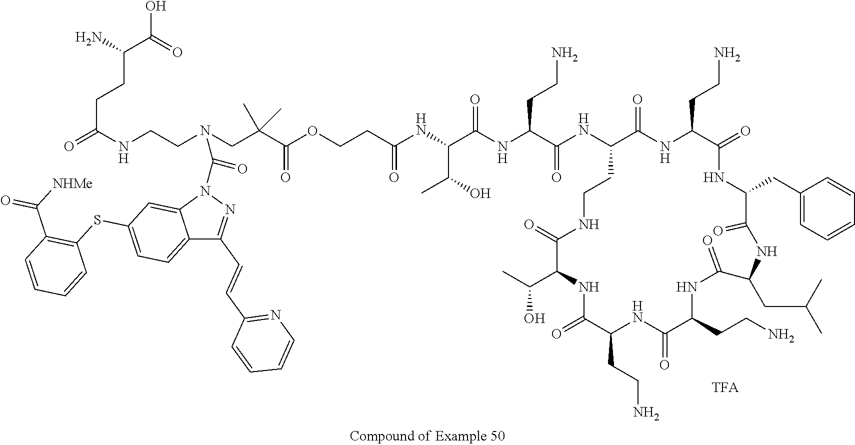 [SMILES: CNC(=O)c1ccccc1Sc1ccc2c(/C=C/c3ccccn3)nn(C(=O)N(CCNC(=O)CC[C@H](N)C(=O)O)CC(C)(C)C(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Compound of Example 50. The compound of Example 50 (TFA salt form) is prepared analogously to the procedures described for the preparation of the compound of Example 29, except using Intermediate 209 instead of the Intermediate 103 at the amide coupling step with PMBN(Boc)₄, and using Intermediate 211 instead of the Intermediate 105 for respective reaction with the Intermediate 43 (which is used in identical manner for the preparation of both the compounds of Examples 29 and 50).

### Example 51

### Synthesis of the Compound of Example 51

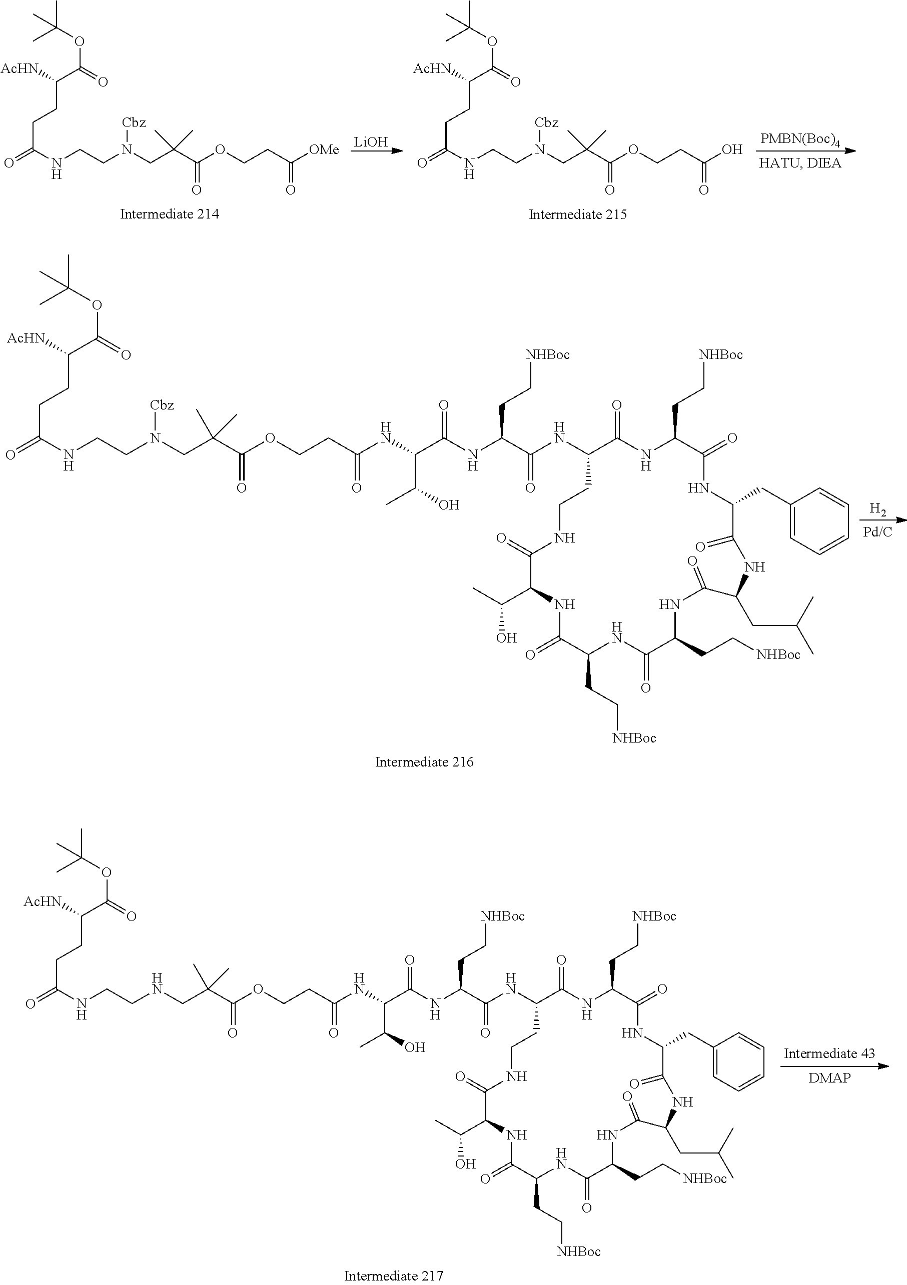

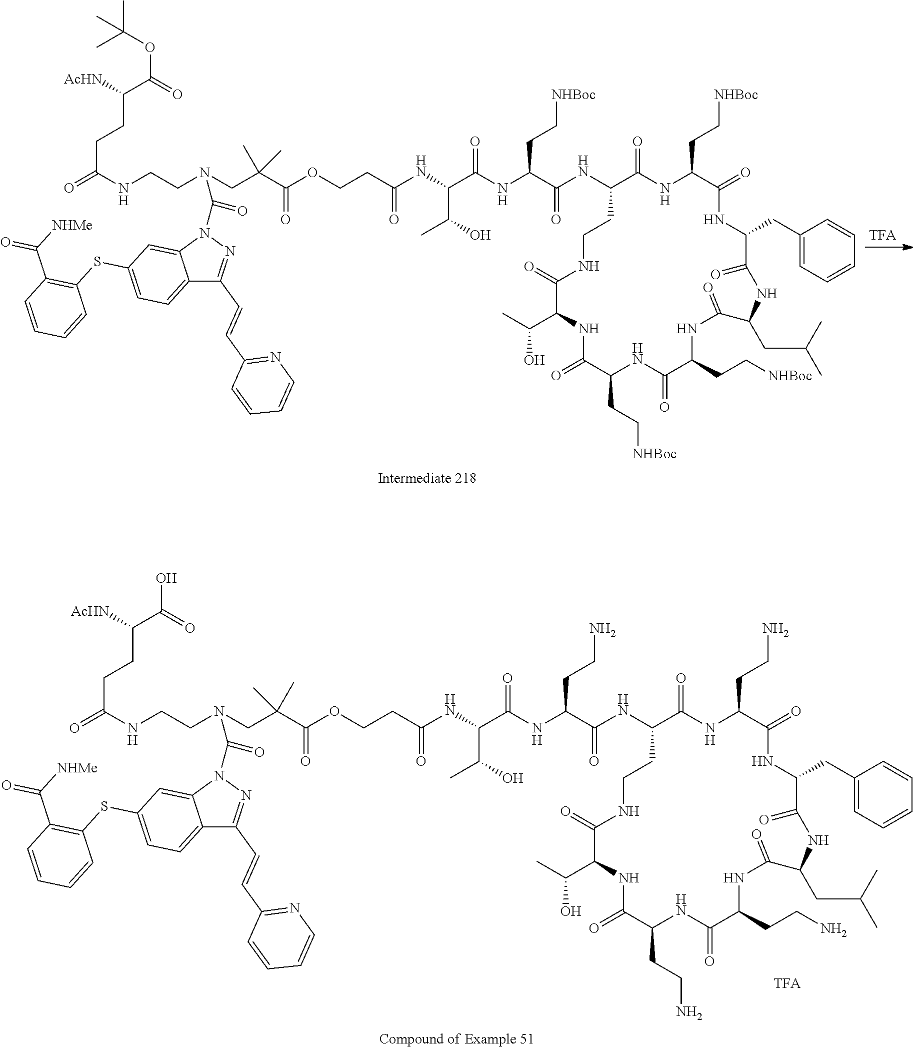

Compound of Example 51. The compound of Example 51 (TFA salt form) is prepared analogously to the procedures described for the preparation of the compound of Example 29, except using Intermediate 215 instead of the Intermediate 103 at the amide coupling step with PMBN(Boc)₄ and using Intermediate 217 instead of the Intermediate 105 for respective reaction with the Intermediate 43 (which is used in identical manner for the preparation of both the compounds of Examples 29 and 51).

### Example 52

### Synthesis of the Compound of Example 52

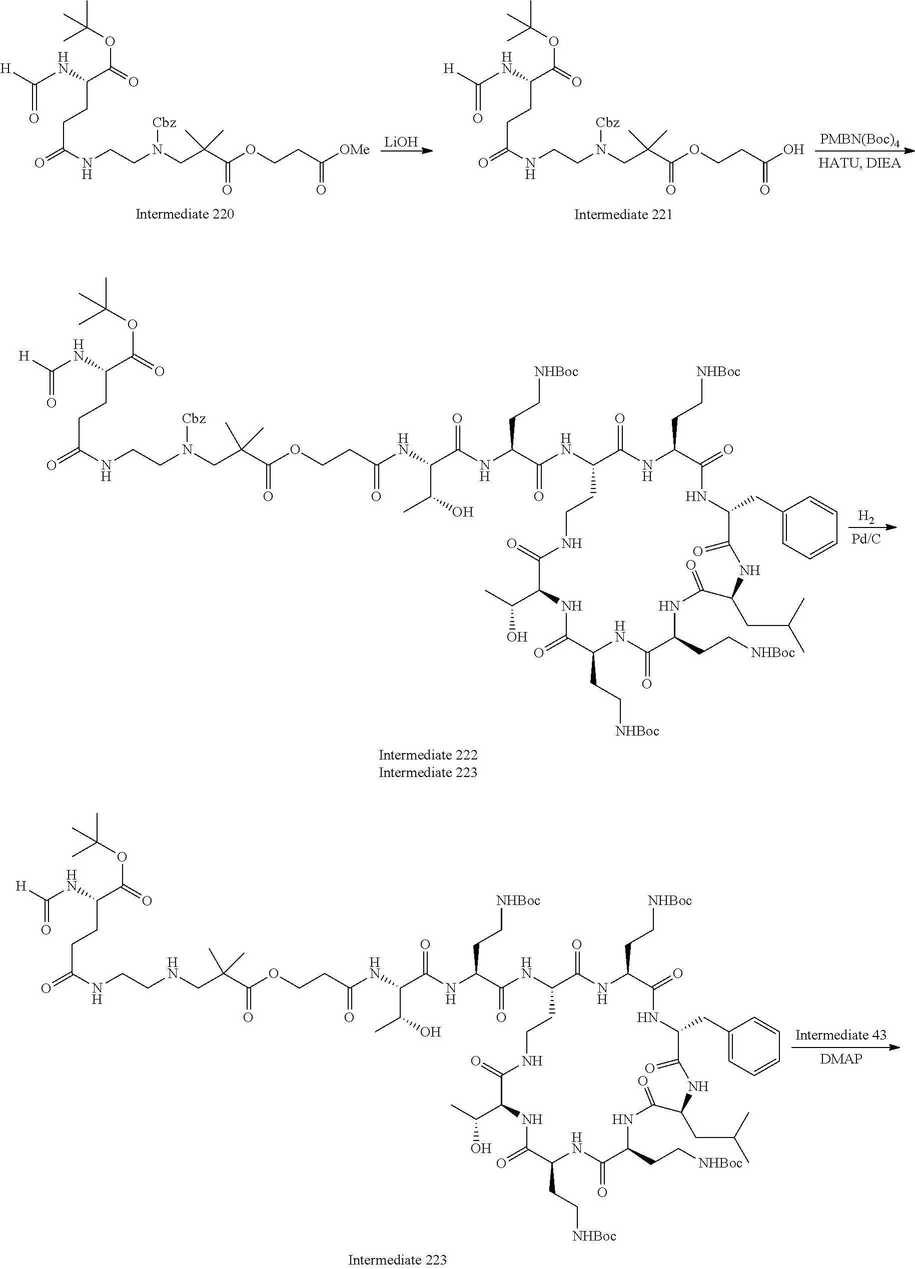

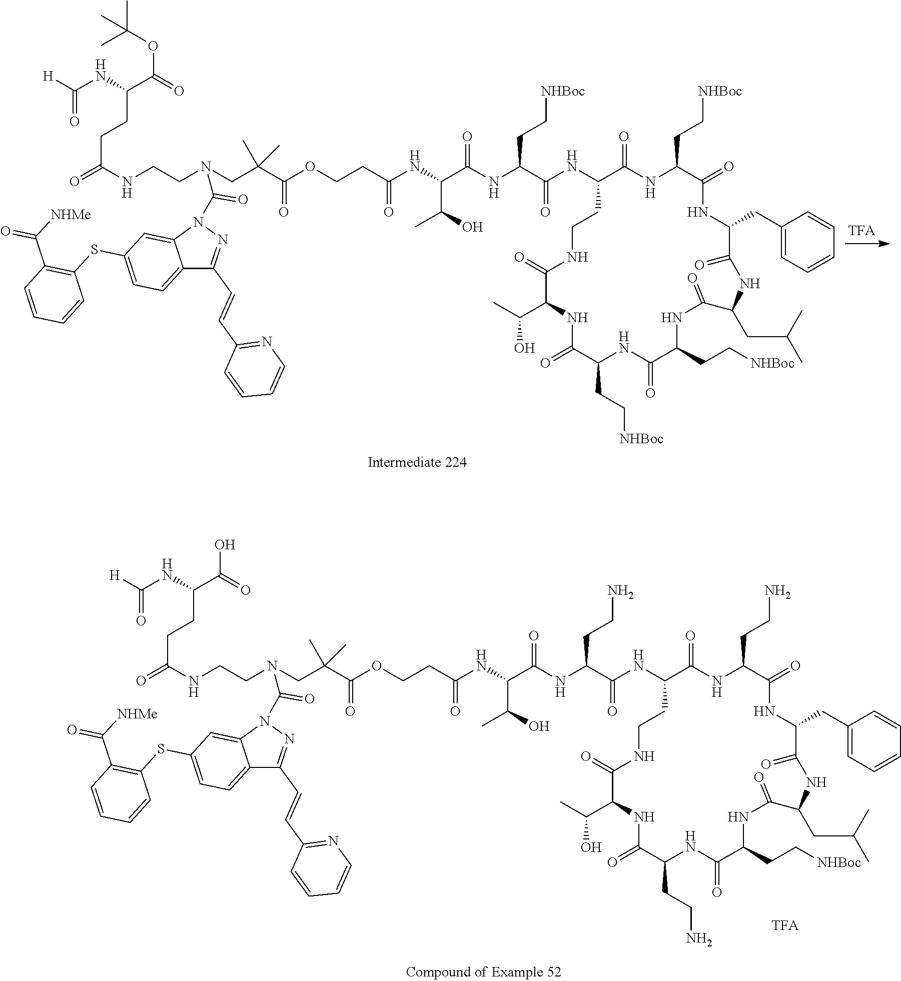

Compound of Example 52. The compound of Example 52 (TFA salt form) is prepared analogously to the procedures described for the preparation of the compound of Example 29, except using Intermediate 221 instead of the Intermediate 103 at the amide coupling step with PMBN(Boc)₄, and using Intermediate 223 instead of the Intermediate 105 for respective reaction with the Intermediate 43 (which is used in identical manner for the preparations of both the compounds of Examples 29 and 52).

### Example 53

### Synthesis of the Compound of Example 53

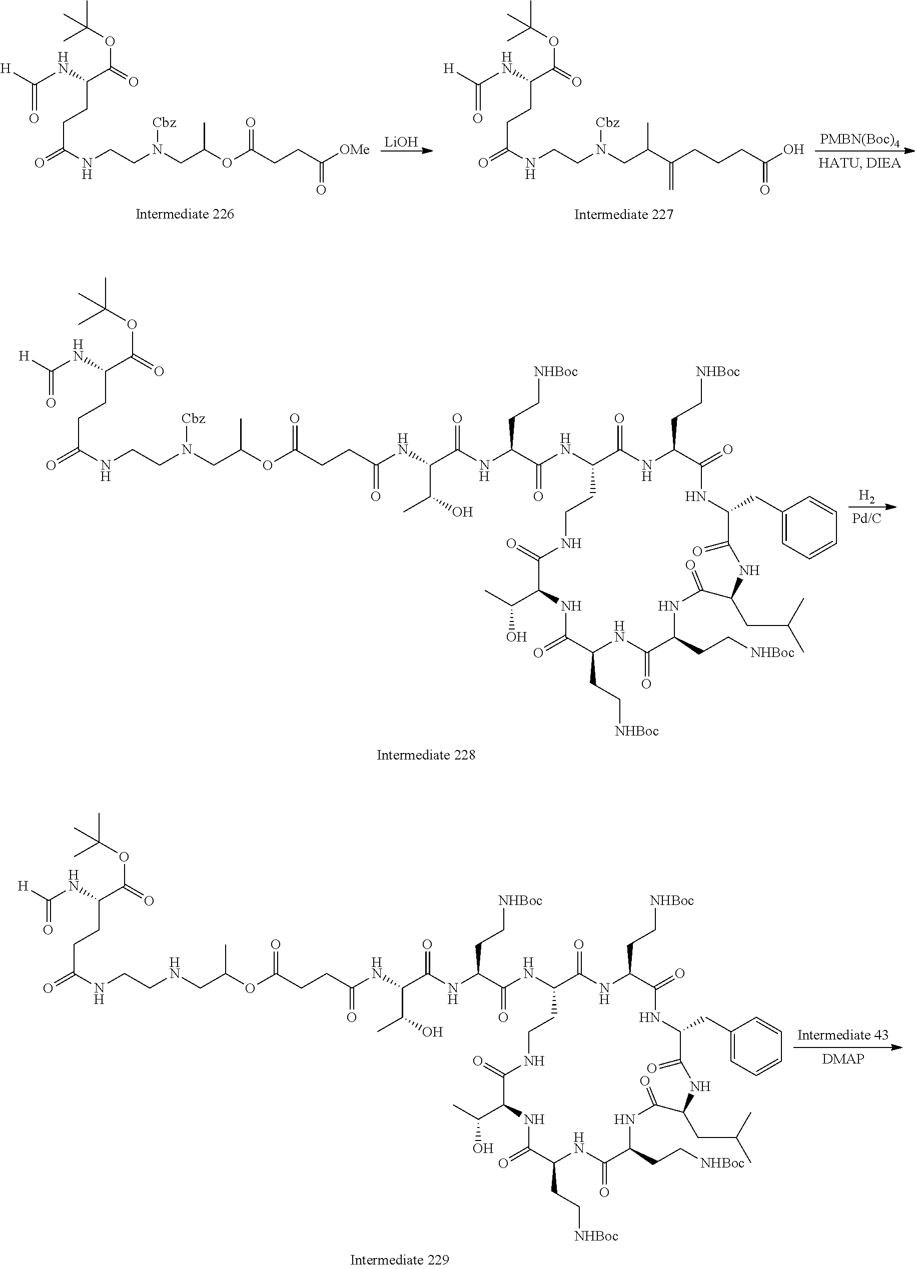

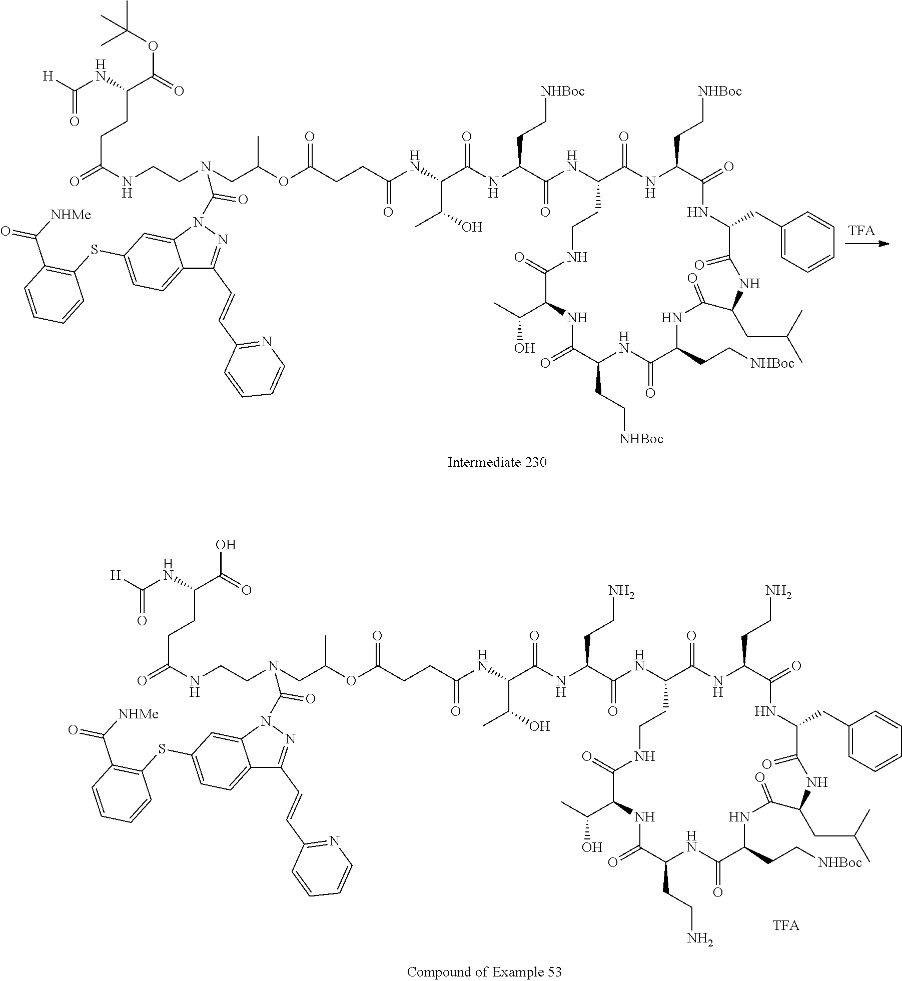

Compound of Example 53. The compound of Example 53 (TFA salt form) is prepared according to the procedures described for the preparation of the compound of Example 29, except using Intermediate 227 instead of the Intermediate 103 at the amide coupling step with PMBN(Boc)₄, and using Intermediate 229 instead of the Intermediate 105 for respective reaction with the Intermediate 43 (which is used in identical manner for the preparations of both the compounds of Examples 29 and 52).

Optionally, the TFA salt of the compound of Example 53 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J Chemical Education. 2005, vol. 82, p. 575); or by dissolution of the TFA salt in aq. media, addition of an excess of aq. HCl, followed by lyophilization or direct evaporation of a solution under vacuum. Resulted solid is optionally recrystallized, for example, from an alcohol-containing media, such as EtOH-EtOAc, or isopropanol-water, or alike solvent system.

### Example 54

### Synthesis of the Compound of Example 54

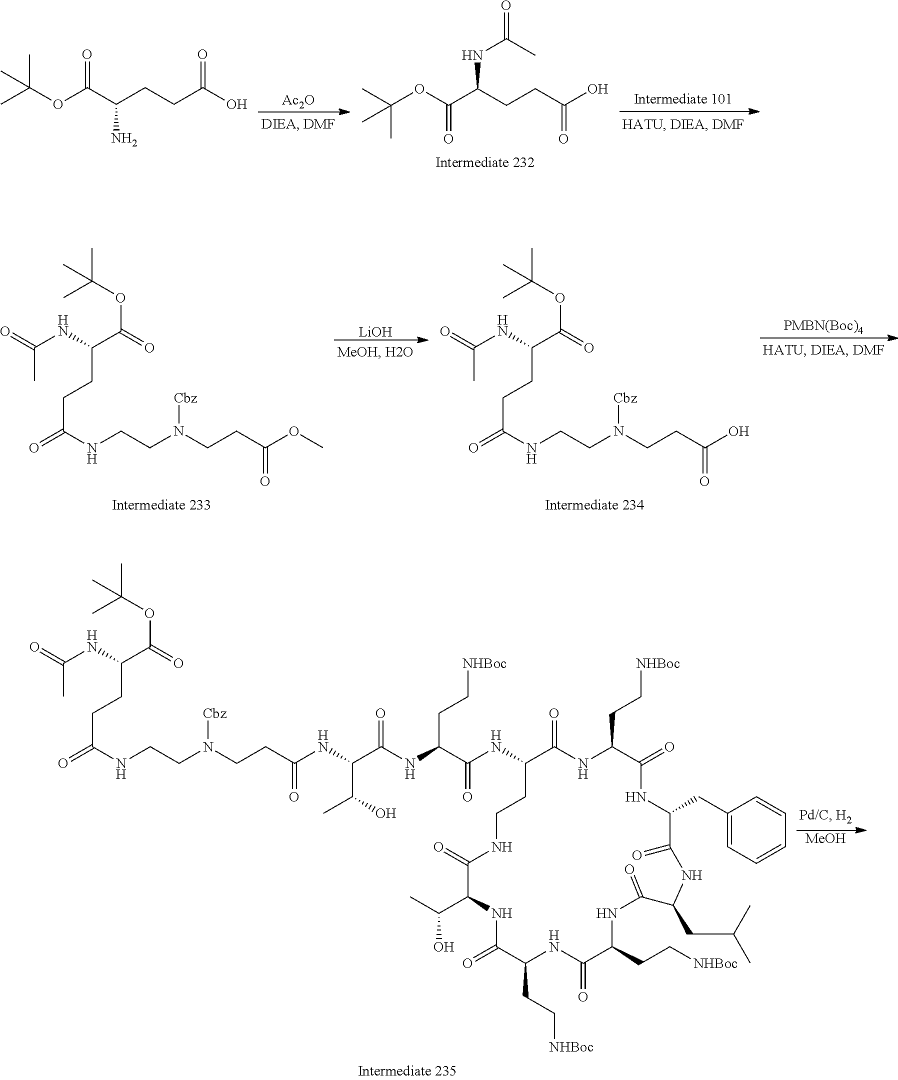

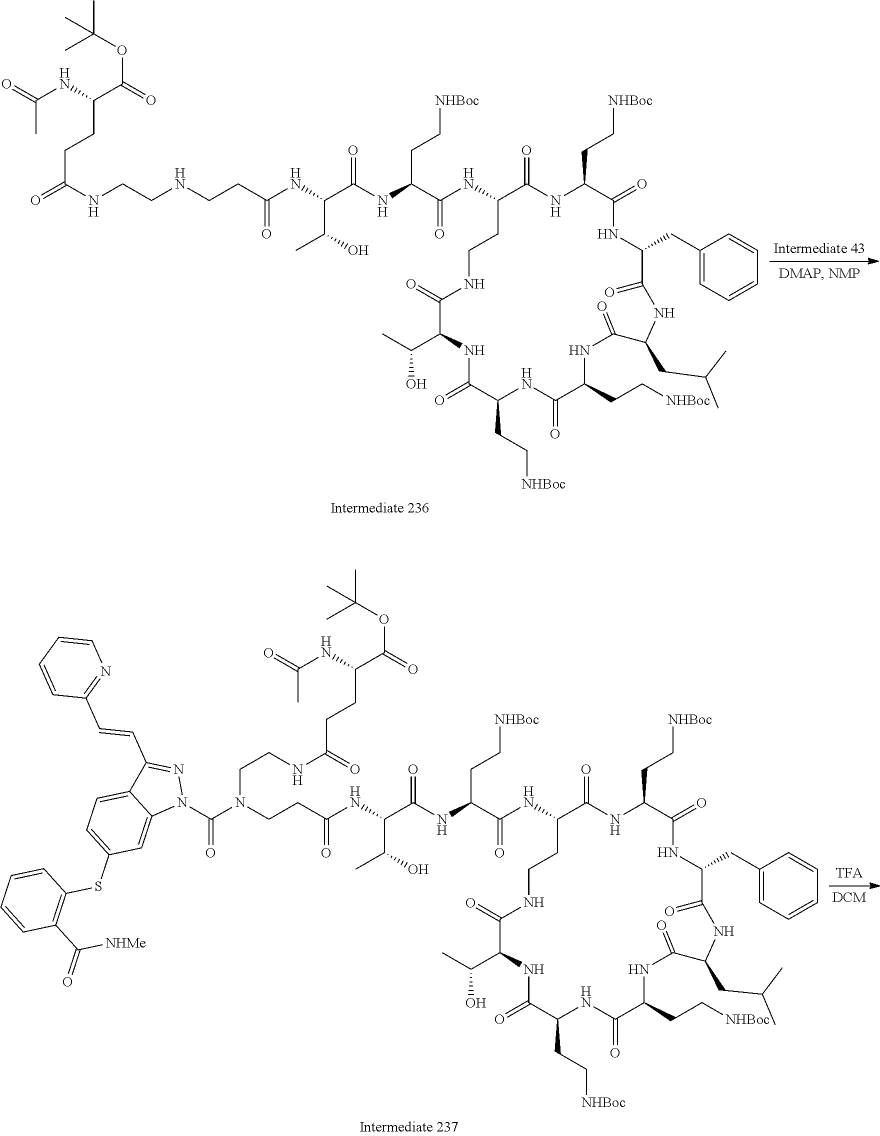

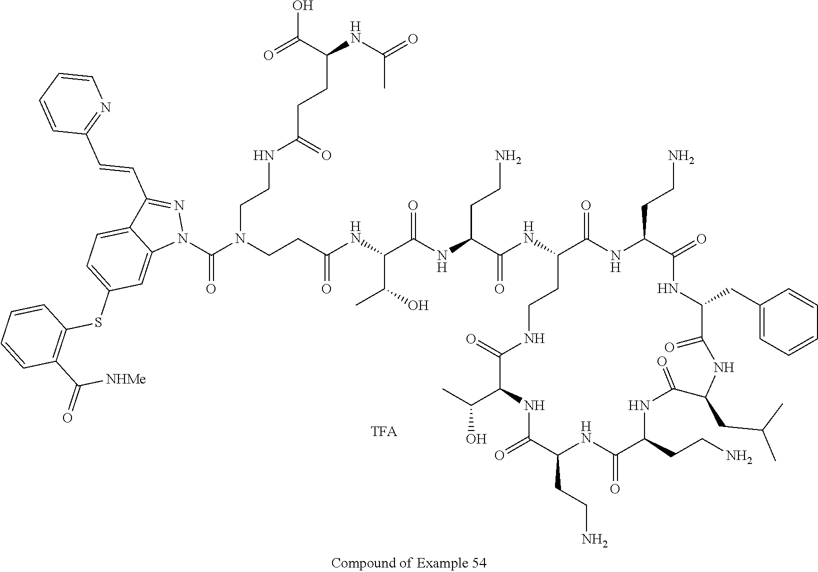 [SMILES: CNC(=O)c1ccccc1Sc1ccc2c(/C=C/c3ccccn3)nn(C(=O)N(CCNC(=O)CC[C@H](NC(C)=O)C(=O)O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1]

Compound of Example 54. The compound of Example 54 (TFA salt form) was prepared analogously to a similar procedure described for the preparation of the compound of Example 29, except using Intermediate 234 instead of the Intermediate 103 at the amide coupling step with PMBN(Boc)₄. At the final step of the procedure, Intermediate 237 (108.0 mg) was converted into the Compound of Example 54, which was isolated as TFA salt: white solid (33.2 mg). MS: 1661.7 [M+H]⁺. NMR δ 8.41 (d, J=5.7 Hz, 1H), 8.24 (t, J=7.9 Hz, 1H), 8.03 (d, J=8.2 Hz, 1H), 7.74-7.66 (m, 1H), 7.66-7.58 (m, 1H), 7.39 (d, J=16.8 Hz, 1H), 7.32-7.23 (m, 4H), 7.06 (dt, J=21.3, 7.7 Hz, 5H), 6.95 (d, J=7.2 Hz, 2H), 4.29 (t, J=8.2 Hz, 1H), 4.20 (dd, J=9.1, 5.2 Hz, 2H), 4.06-3.97 (m, 4H), 3.97-3.93 (m, 2H), 3.93-3.87 (m, 3H), 3.52 (s, 1H), 3.30 (s, 1H), 3.11-3.03 (m, 2H), 2.87-2.73 (m, 9H), 2.60 (d, J=8.5 Hz, 1H), 2.51 (d, J=14.7 Hz, 2H), 2.47 (s, 2H), 1.95 (dt, J=15.8, 7.8 Hz, 6H), 1.66 (dd, J=9.8, 5.2 Hz, 3H), 1.36 (s, 1H), 1.06 (s, 6H), 0.89 (t, J=7.1 Hz, 7H), 0.48 (d, J=5.8 Hz, 3H), 0.40 (d, J=5.8 Hz, 3H).

Optionally, the TFA salt of the compound of Example 54 is converted into an HCl salt, a H₂SO₄ salt, a citric acid salt, a lactic acid salt, a mandelic acid salt, or another pharmaceutically acceptable salt. As generally applicable, this is accomplished by a standard ion-exchange process using an HCl (or another acid) form of an anion-exchange resin (as described, for example, by Elder in J. Chemical Education. 2005, vol. 82, p. 575); or by dissolution of the TFA salt in aq. media, addition of an excess of aq. HCl, followed by lyophilization or direct evaporation of a solution under vacuum. The resulting solid is optionally recrystallized, for example from an alcohol-containing media, such as EtOH-EtOAc, or isopropanol-water, or similar solvent system.

### Utility and Testing

The compounds provided herein exhibit a pronounced therapeutic effect (efficacy) against a variety of kidney cancers, including RCCs and mRCCs. Therefore, these agents are useful for a targeted therapy of kidney-associated cancers.

The novel compounds provided herein comprise an anticancer-bioactive molecule generally conjugated with a carrier peptidic fragment(s) (for example, a polymyxin cyclopeptide derivative). The latter serves as a carrier for a delivery of such a compound into kidneys, due to the unique ability of said peptidic fragment to bind kidney tissues.

First, some of the compounds provided herein exhibit innate activity against cancer cells (or anticancer cytotoxicity) as intact molecular structures. Such intrinsic activity is inherent in said molecules, and this activity does not rely on a metabolic release of an anticancer agent conjugated within the structures (in other words, covalently connected to a peptidic fragment that serves as a carrier for delivery of a compound into kidney, due to the propensity of said peptidic part to bind kidney tissues).

Secondly, certain compounds provided herein exhibit modest or no innate anticancer cytotoxicity as intact molecules. Upon administration, these accumulate in kidneys and then are metabolized in the organ affected by renal cancers. This metabolism leads to a release of an anticancer drug (or a cytotoxic agent), which was incorporated within the administered compound, at the site of a cancer, to result in anticancer therapeutic effect (manifested, for example, in a cancer tumor size reduction, or a stopped tumor growth). Importantly, this metabolic degradation occurs selectively: the active entity is released in the requisite drug form (without metabolic alteration that could reduce the required anticancer activity).

Thirdly, certain compounds provided herein combine the innate anticancer activity as intact molecules with an anticancer effect resulting from a metabolic release of active anticancer drug (a cytotoxic agent) which was incorporated within the administered molecule. This combined effect can be either additive or synergistic in nature. This modality comprises a dual-action mode: the innate activity of the intact conjugate compound, beneficially coupled with the activity of a metabolically-released drug (bioactive) incorporated within such administered conjugate.

Important, all three therapeutic modalities described above are exhibited upon a selective or targeted renal delivery of the compounds provided herein. In other words, the compounds administered to a mammal in the need of a therapy rapidly accumulate in the kidneys affected by a renal cancer.

Preferential accumulation of compounds provided herein in kidneys (or near the site of a kidney cancer) may be evaluated by pharmacokinetic (PK) tests, such as in a standard rat PK test. PK data are generally used to establish the key parameters predictive of the therapy outcome, such as drug concentration (C) at given time points, drug concentration at the target tissue(s) (C_{Target}), area under the curve (AUC) for a plot monitoring the change in the systemic drug concentration over time, and other parameters. Thus, drug concentration in organ (or body compartment) affected by cancer is important for effective action of anticancer agents (as described, for example, by Zhang et al. in Drug Metabolism and Disposition. 2019, vol. 47, p. 1122).

Representative compounds provided herein have been tested in a rodent PK model of intravenous administration performed analogously to methods described in the monograph Current Protocols in Pharmacology, 2005, 7.1.1-7.1.26, John Wiley & Sons, Inc.

In PK studies, the levels (concentration) of a therapeutic drug is determined in key body compartments, such as blood and select organ tissues, is determined over a given time course. The levels of an active compound in the organ affected by a disease is of particular importance, since such a compound is intended to target the disease therein. For therapy of renal cancers, the targeted organ is kidney.

Anticancer efficacy (in vivo activity) depends on and is directly tracked to certain required levels of an anticancer drug in the mammal in the need of therapy (as reviewed, for example, by Fogli et al. in Cancer Treatment Reviews. 2020, vol. 84, 101966; by Hu-Lowe et al. in Clin. Cancer Research. 2008, vol. 14, p. 7272; and by Zhang et al. in Drug Metabolism and Disposition. 2019, vol. 47, p. 1122). This concentration-therapy relationship is rooted in a mode of action of anticancer drugs, generally based on a concentration-dependent inhibition of the cancer cell growth (for example, cancer cells manifested as a tumor).

Thus, incomplete or no inhibition of cancer cells (or tumor growth) could result, if a drug concentration is too low to achieve said inhibition. This generally leads to ineffective therapy, often aggravated with an increased risk of developing a cancer drug-resistance, with latter rendering the disease non-responsive to a drug (as reviewed, for example, by Komarova et al. in PNAS. 2005, vol 102, p. 9714). Conversely, if a drug concentration at a targeted organ is higher, then an enhanced anticancer efficacy is generally observed, and with a minimized risk of developing cancer drug-resistance.

For example, an efficacy effect of the renal cancer drug axitinib is reliably forecasted from its concentration in the blood, with the blood circulation encompassing kidneys affected by the disease (as reviewed by Hu-Lowe et al. in Clin. Cancer Research. 2008, vol. 14, p. 7272). Specifically, the total axitinib blood concentration of about 40 ng/mL was reported as the marker (predictor) of high therapeutic efficacy against renal carcinomas. Therefore, if the drug concentration falls below 40 ng/mL, a reduced therapeutic efficacy is anticipated (Hu-Lowe et al. in Clin. Cancer Research. 2008, vol. 14, p. 7272). Upon axitinib administration, kidney drug concentrations generally do not exceed the levels of this drug in blood (and often are lower for kidney, relative to that in blood; see, for example, Table 2 below).

Therefore, axitinib kidney concentrations of 40 ng/mL or about 39 ng/g (per kidney tissue density of 1.03 g/mL) is generally predictive of an effective inhibition of renal cancers, needed for a successful therapy of the mammal in the need thereof.

Illustrative PK data for compounds of Examples 5, 6, and 15 are summarized in the Table 1 below. As clear from the data for the rat PK model, these compounds exhibit an effective ability to target kidneys, as confirmed by high levels of preferential accumulation thereof in kidney tissues, the organ for a targeted therapy of this invention.

TABLE 1
Targeted delivery of compounds herein
to kidneys, compared to blood.

| | Blood Plasma | Kidney Tissue | Kidney-to-Blood |
|---|---|---|---|
| EXAMPLES | Conc.,^{a} μmol | Conc.,^{a, b} μmol | Conc. Ratio |
| Example 5 | BLQ^{c} | 1.88 | >20 |
| Example 6 | 0.11 | 34.93 | 318 |
| Example 15 | 0.19 | 5.13 | 27 |

^{a}At T = 6 h post-administration by injection into a rat tail vein, 3 mg/kg.
^{b}Corrected for kidney tissue density 1.03 mg/mL.
^{c}BLQ: observed value below the level of quantitation.

Additional PK data in rodents for other compounds provided herein are illustrated in the Table 2 below. The data indicated a targeted delivery of anticancer drug axitinib, preferentially released from these compounds into kidneys.

TABLE 2
Targeted delivery of renal cancer drug axitinib using exemplary
compounds herein: the drug concentration in kidneys compared
to blood, at test time (T, h) points post-administration.

| | Blood Plasma | Kidney Tissue |
|---|---|---|
| EXAMPLES | Conc.,^{a} ng/mL | Conc.,^{a} ng/g |
| Axitinib^{b} | T = 4 h: 95.1 | T = 4 h: BLQ^{c} |
| | T = 6 h: 35.4 | T = 6 h: BLQ |
| Example 29 | T = 3 h: 14.9 | T = 3 h: 371.6 |
| | T = 6 h: BLQ | T = 6 h: 288.8 |
| Example 31 | ND^{d} | T = 3 h: 142.4 |
| | ND | T = 6 h: 91.0 |
| | ND | T = 12 h: 37.0 |
| Example 32 | ND | T = 3 h: 162.6 |
| | ND | T = 6 h: 62.9 |
| | ND | T = 12 h: 41.6 |
| Example 35 | ND^{d} | T = 3 h: 178.5 |
| | ND | T = 6 h: 122.3 |
| | ND | T = 12 h: 96.8 |
| Example 40 | ND | T = 3 h: 103.5 |
| | ND | T = 6 h: 79.7 |
| | ND | T = 12 h: 77.6 |
| Example 41 | ND | T = 3 h: 44.3 |
| | ND | T = 6 h: 44.5 |
| | ND | T = 12 h: 34.4 |
| Example 43 | T = 3 h: BLQ | T = 3 h: 204.3 |
| | T = 6 h: BLQ | T = 6 h: 199.7 |
| | T = 12 h: BLQ | T = 12 h: 263.0 |
| Example 45 | ND | T = 6 h: 55.1 |
| | ND | T = 12 h: 58.0 |
| | ND | T = 24 h: 42.5 |

^{a}Agent administered through an injection, as a single dose of 3 mg/kg; rat model for the compound of Example 45, and mouse model for other compounds.
^{b}Administered via oral gavage, a single dose of 10 mg/kg.
^{c}BLQ: observed value below the level of quantitation.
^{d}ND: not determined.

Specifically, administration of the compounds provided herein allows to achieve beneficially higher levels of the therapeutic agent in kidneys, as opposed to administration of axitinib in its standard free drug form. In contrast, the administration of axitinib itself results in its high level in blood, which is the key reason for off-target adverse effects of axitinib and other drugs in therapy of renal cancers (as reviewed, for example, by Fogli et al. in Cancer Treatment Reviews. 2020, vol. 84, 101966).

As noted already, the axitinib efficacy is predicted from the total drug concentration in plasma of at least 40 ng/mL (as reviewed by Hu-Lowe et al. in Clin. Cancer Research. 2008, vol. 14, p. 7272), corresponding to 39 ng/g for the concentration in kidneys (normalized per kidney tissue density of 1.03 g/mL). According to experimental data in Table 2, the compounds of Examples 29-32, 35, 40, 41, 43, and 45 all effectively and selectively deliver axitinib into kidneys, with this drug released from said compounds at levels of well over 39 ng/g. The data are indicative of an effective therapy of kidney cancers with the compounds provided herein.

Also apparent from above data, the exemplary compounds provided herein selectively deliver the renal cancer drug into kidneys (the disease site), and at levels markedly exceeding the kidney levels achieved using axitinib itself. For example, only trace (BLQ, below levels of quantitation) axitinib concentration was detected in rodent kidneys at 4 h post-administration of the drug (Table 2). In contrast, above-therapeutic >39 ng/g levels of the drug delivered into kidney by administration of compounds of Examples 29-32, 35, 40, 41, 43, and Example 45 persisted throughout representative PK time points: 3-12 h in mouse tests, and tested up to 24 h in rat (tested for the compound of Example 45). For example, axitinib kidney levels achieved through administration of the compound of Example 29 was 288.8 ng/g at the time point of 6 h. In contrast, the kidney levels of axitinib observed for its standard oral form were too low to be quantified (BLQ), as determined just after 4 h after administration of the drug. The data indicates greatly increased efficacy potential for the compounds provided herein, as compared to the standard axitinib therapy of renal cancers.

Remarkably, these enhanced axitinib kidney levels were achieved with significantly reduced dosing (for the content of the active drug conjugated within the compounds provided herein). This is exemplified, by the data (Table 2) for the compound of Example 29 administered at 3 mg/kg, in contrast to much higher 10 mg/kg dose of axitinib administered in its standard free drug form. Notably, the molecular weight (MW) for axitinib is 386.5 Daltons, while the MW for Example 29 is 2,188/0 (base MW 1617.9; the total MW of 2.188.0 for the isolated trifluoroacetate salt form). Taking into account the relative amount of (conjugated) axitinib in the compound of Example 29 is only about 0.18 (Axitinib MW/Example X MW=386.5/2,188=0.18), the levels of the drug in kidney was achieved with about 5% (0.05 or 1/20) of the standard axitinib dose (3 mg/kg:10 mg/kg x 0.18×100%=5%). This result illustrates a remarkable and very surprising ability of the composition herein to provide beneficially elevated levels of the anticancer drugs at the target organ, achieved with only a small fraction of the total active administered in a form of compounds herein, such as Example 29 (as compared to a standard dose of axitinib required for a renal cancer therapy).

The markedly improved drug levels at the site of kidney cancers (achieved with for exemplary compounds) are indicative of the improved efficacy (in vivo activity) of said compounds, as compared to a standard administration of axitinib. Important, this could be achieved with a reduced amount of axitinib administered in the form of a conjugate compound provided here (such as the compound of Example 29).

In addition, these favorable and surprising PK data indicate an option of less frequent administration and/or reduced dosing of compounds provided herein, as compared to axitinib. For example, a standard twice-daily administration of axitinib could be substituted by a once-daily or once-weekly administration of a compound provided herein. This provides a significant convenience to a patient under therapy for a kidney cancer, as well as pharmacoeconomic potential resulting from beneficially minimized number of hospital visits. Separately, the selective (or targeted) kidney delivery of compounds provided herein comprises a significant safety benefit. A standard therapy with cytotoxic cancer drugs is generally accompanied by significant adverse effects. For example, axitinib therapy suffers from multiple adverse effects noted as Warnings in the prescribing information for the drug (marketed as Inlyta). In particular, hypertensive adverse effects were reported (for example, by Fogli et al. in Cancer Treatment Reviews. 2020, vol. 84, 101966), with an incidence of 40-64%, including a hypertensive crisis. Principally, these adverse effects results from high levels of axitinib circulating in the blood, and thus distributed into vital organs not affected by renal cancers.

Indeed, the PK data of Table 2 indicate much higher levels of the drug in blood as compared to kidneys, after a standard administration of axitinib to a mouse, as apparent from the data for time points of 2 and 4 h. As a result, the drug exerts adverse cytotoxic effects—often referred to as “off-target activity”—in healthy organs not intended for such cancer therapy.

In contrast, the administration of exemplary compounds provided herein results in greatly minimized amounts of a (released) active drug in blood, and beneficially concomitant with a selective (targeted) delivery of the drug into kidneys. Therefore, a greatly reduced off-target activity (toxicity) is anticipated for therapy with a compound provided herein, as compared to a standard drug with anticancer agent, such as axitinib.

In vitro activity of compounds provided herein may be assessed by standard testing procedures using various cancer cell lines (alongside normal cell comparators), such as described, for example, in ACS Pharmacol. Transl. Sci. 2019, vol. 2, p. 18; J. Med. Chem. 2018, vol. 61, p. 5304; and methods in references cited therein.

It is important to distinguish between in vitro activity (potency) from in vivo activity (efficacy). In vitro tests allow for the tested compound to interact with cancer cells directly, typically, by introducing the test compound into cancer cells suspended in a solution of nutrients that allow for the cell growth.

In contrast to in vitro tests, in vivo evaluation entails administration of a compound to a mammal (such as a rodent), for example, intravenously. The compound is then circulated in the blood and is distributed in organs and tissues. This distribution can occur with varying efficiency for different organs, and may result in drug accumulation in some organs, concomitant with low levels of same drug in other organs. Importantly, in this process, the compound is exposed to numerous proteins and enzymes (such as esterases and peptidases) that may metabolize (degrade) the compound during in vivo test.

For example, some compounds provided herein are metabolized in vivo to release the active drug molecule conjugated within said compounds using metabolically cleavable linkers. As a result, such compound that has no or modest innate or intrinsic activity (potency) when tested in vitro may exhibit a high in vivo activity (efficacy) when tested in vivo.

The anticancer activity in vitro (potency) of exemplary compounds provided herein are illustrated by the data in Table 3. Therein, Caki-1 is a human clear cell renal cell carcinoma (ccRCC) line. The ACTHN cell line was derived from the pleural effusion of a 22 year-old male patient with metastatic renal carcinoma. The 786-O cell line is a hypertriploid renal cancer cell carcinoma cell line. Generally, cells were plated into 120-well microplates and pre-incubated for 24 h. Subsequently, the test compound solutions were added, and cells growth inhibition analyzed with 9-point data plot. After 72 h of the incubation, the number and proportion of the viable cells was determined by CTG assay, and IC₅₀ values calculated.

TABLE 3
In vitro activity (potency) of exemplary
compounds against cancer cells.

| | Parent agent | | | |
|---|---|---|---|---|
| | conjugated | Inhibition of cancer cells, IC₅₀, μmol | | |
| EXAMPLES | within Example | Caki-1 | ACHN | 786-O |
| Axitinib | NA | 28.7 | 9.3 | 28.9 |
| Sunitinib | NA | 6.9 | NT | NT |
| Pazopanib | NA | 19.4 | NT | NT |
| Example 6 | Sunitinib | 16.1 | NT | NT |
| Example 15 | Pazopanib | 22.7 | NT | NT |
| Example 17 | Axitinib | 8.6 | NT | NT |
| Example 27 | Axitinib | 6.3 | 7.3 | 8.4 |
| Example 28 | Pazopanib | 17.0 | NT | NT |
| Example 29 | Axitinib | >30 | 28.0 | >30 |
| Example 30 | Axitinib | 5.6 | 9.5 | 10.8 |
| Example 31 | Axitinib | 5.2 | 15.4 | 9.8 |
| Example 32 | Axitinib | 9.8 | 20.0 | 14.6 |
| Example 33 | Axitinib | 11.5 | NT | NT |
| Example 35 | Axitinib | 8.3 | NT | NT |
| Example 38 | Sunitinib | 3.3 | 5.5 | 5.0 |
| Example 39 | Axitinib | 5.9 | 13.0 | 10.1 |
| Example 40 | Axitinib | 14.1 | NT | NT |
| Example 41 | Axitinib | 11.1 | NT | NT |
| Example 42 | Sunitinib | 6.8 | NT | NT |
| Example 43 | Axitinib | 12.4 | 12.8 | 12.5 |

The in vitro anticancer activity of compounds provided herein is surprising. While these incorporate an anticancer drug structure within the new chemical composition, the anticancer moiety connected therein is dramatically altered, as compared to the (highly optimized) original anticancer drug structure when not conjugated, e.g. axitinib.

For example, in the compounds of Examples 29-32, 35, 40, 41, 43, and Example 45, the structure for the axitinib drug moiety incorporated therein has been altered. Specifically, within these novel conjugates, the axitinib fragment lacks the heterocyclic (indazole) NH group. Important, the NH hydrogen-binding interactions have been reported as essential for the binding of axitinib at the active center of cancer EGFR enzymes, the key mode for inhibiting cancer cells by this drug (described, for example, in Molecules. 2018, 23, 747). Thus, it is important for the inhibitory activity of axitinib against cancer cells. In the composition provided herein, the compact NH group at indazole heterocycle was replaced by a highly branched urea fragment, further substituted with a large polymyxin cyclopeptide. The dramatic structure alteration is illustrated by the fact that, in the compound of Example 43, the polypeptide substructure newly added to the original drug moiety exceeds the molecular weight (MW) for axitinib over three-fold: 386.5 and 1,273.3 (1,659.8-386.5=1,273.3) Daltons for axitinib and the peptidic addition (in the conjugate) respectively. In the face of this substantial structural alteration, it is very surprising that the compound of Example 43 is actually over 2-fold more active against the cancer cells, such as Caki-1 and 786-O, than the axitinib itself (see the data in the Table 3: IC₅₀ ranges 28.7-28.9 and 12.4-12.5 μmol for axitinib and the compound of Example 43, respectively). The activity of the compound of Example 29 to inhibit ACHN cancer cells with in vitro potency comparable to that for axitinib is likewise surprising (see Table 2: IC₅₀ values 28.0 and 9.3, respectively).

The innate anticancer activity of novel axitinib-polypeptide conjugates provided herein is especially surprising in face of the highly restrictive structure-activity relationships (SAR) for its close analogs. As reported, for example, in Molecules. 2018, 23, 747, multiple isosteric designs closely mimicking axitinib structure have failed to replicate the activity of this drug, and exhibited many-fold reduced inhibition of the targeted cancer enzymes (such as VEGFR-2 kinase), as compared to axitinib itself. Specifically, precluding the NH hydrogen-binding by way of replacing these groups with NMe was found to be detrimental for inhibition of the cancer enzyme, such as VEGFR-2 enzyme. Thus, the in vitro anticancer activity indicative of an intrinsic ability of compounds provided herein to inhibit cancer cells is not at all anticipated.

As stated above, certain compounds of this invention possess a reduced in vitro cytotoxicity against cancer cells, resulting in a beneficially reduced off-target activity(ies) against healthy organs (with said off-target effect being responsible for most adverse effects in the standard cancer therapy). While exhibiting a reduced cytotoxicity of the intact conjugate molecule in vitro, such compounds exert therapeutic anticancer effect in vivo after being metabolized with release of the active drug (conjugated within the administered molecule) at the target organ of a cancer. Thus, the administration of such a compound (which may be inactive in vitro) to a mammal in the need of cancer therapy, results in a selective targeted delivery therapy, with a pronounced anticancer activity observed in vivo.

Data for representative compound of Example 29 are illustrated in Table 4. This compound exemplifies several favorable aspects achieved for the composition provided herein.

TABLE 4
In vitro activity (potency) of exemplary
compounds against cancer cells.

| | In vitro inhibition data | | | | | In vivo axitinib |
|---|---|---|---|---|---|---|
| | | | | Non-cancer | | conc. at T = |
| | Cancer cells, | | | kidney | | 6 h post- |
| EXAM- | IC₅₀, μmol | | | cells, IC₅₀, μmol | | administra- |
| PLES | Caki-1 | ACHN | 786-O | HK-2 | HEK-293 | tion,^{a} ng/g |
| Axitinib | 28.7 | 9.3 | 28.9 | 22.9 | 8.7 | BLQ^{b} |
| Example | >30 | 28.0 | >30 | NT | 14.4 | 288.8 |
| 29 | | | | | | |

^{a}Single dose administration to mouse: 10 mg/kg oral gavage for axitinib; 3 mg/kg intravenous for Example X.
^{b}BLQ: observed value below the level of quantitation.

First, this compound of Example 29 preferentially delivers the drug axitinib (conjugated within the structure of Example 29) into the kidney, the organ affected by renal cancers (with no or minimal release of the drug in blood, see Table 2). This indicates a surprisingly reduced potential for off-target toxicity typical for a standard administration of the drug axitinib. Secondly, this exemplary compound exhibits significantly minimized cytotoxicity in both cancer and non-cancer (healthy) kidney cells when compared to axitinib. For example, its cytotoxicity against human HEK-293 embryonic kidney cells is markedly—by over 50%—reduced, as compared to axitinib: IC₅₀ 14.4 and 8.7 μmol, for the compound of Example 29 and axitinib, respectively. Finally, it delivers axitinib into kidneys much more efficiently than the drug itself is delivered. Indeed, the levels for axitinib released from this agent are determined at 288.8 ng/g, or over 7-fold of the value of 39 ng/g blood levels necessary for the therapeutic effect of axitinib against renal (and other) cancers.

Surprisingly, while the compound of Example 29 incorporates a polymyxin structure, it exhibits a minimized antibacterial activity (measured in vitro as MIC, minimum inhibitory concentration) compared to typical antibacterial activity for the polymyxin antibiotics such as polymyxin B and colistin, with at least 2 to 4-fold higher MIC (reduced antibacterial potency), as compared to these drugs. The antibacterial activity is entirely different from anticancer activity, and it is not intended nor desired for therapy of kidney cancers. This further underscores a high selectivity of the compound of Example 29, which specifically targets kidney cancers, with greatly minimized activity against non-cancerous cells, both in vitro and in vivo.

In vivo activity of compounds provided herein may be assessed by testing procedures such described, for example, in J. Vis. Exp. 2014, (86), e51485; Experimental & Molecular Medicine. 2018, vol. 50, p. 30; and methods in references cited therein.

Surprisingly, certain compounds provided herein, when tested in rodent kidney cancer model with intravenous (IV) administration at a dosing (molar amount) equal to a standard therapeutic dosing (molar amount) of axitinib, brivanib, pazopanib or sunitinib, exhibit at 2-fold or higher efficacy, as compared to the standard therapeutic dosing of axitinib, brivanib, pazopanib or sunitinib, with said therapeutic effect determined as a slowed, stopped, or reversed progression of cancer (for example, determined per changes in a cancer tumor size, and/or by using biochemical biomarkers for cancer monitoring, or similar methods such described in such described, for example, in J. Vis. Exp. 2014, (86), e51485; Experimental & Molecular Medicine. 2018, vol. 50, p. 30).

In addition the high efficacy against renal cancers, such drug must be well-tolerated. Surprisingly, while possessing a high anticancer efficacy in mammals, the compounds provided herein exhibit reduced or no toxicity against non-cancerous kidney cells, both in vitro and in vivo a live mammal (rodent) model. For example, the compound of Example 29, the novel targeted delivery axitinib conjugate, was well-tolerated in 14-days repeat-dose mouse tolerability tests, when administered to the test animals at a dose of at least 18 mg/kg/day. This dosing was several-fold higher dose than that required to achieve above-therapeutic axitinib levels in kidneys (and much higher than in for a standard administration of the parent drug axitinib, as discussed above at Table 2).

An improved safety profile of the compounds of the current invention is further established in biomarker assays predictive of nephrotoxicity. Several such assays (including NGAL assay) have been described, for example, by Keirstead et al. in Toxicol. Sci. 2014, vol. 137, pp. 278-291.

Surprisingly, certain compounds provided herein, when administered to a mammal at a dosing (expressed in molar amount) equal to a standard therapeutic dosing (molar amount) of axitinib, brivanib, pazopanib, sunitinib, tivozanib exhibits at least 2-fold reduced rate (frequency or incidence) of adverse effects and/or off-target toxicity manifestation (such as myelosuppression or bone marrow toxicity), as compared to the standard therapeutic dosing of axitinib, brivanib, pazopanib, or sunitinib (for example, as determined by the platelet and/or other blood cells count for myelosuppression or bone marrow toxicity).

Thus, certain compounds of this invention exhibit high anticancer efficacy, but do not suffer from excessive off-target toxicity affected organs not affected by kidney cancers, and exhibit little or no nephrotoxicity against normal kidney cells.

Therefore, unprecedented types of novel compounds and compositions provided herein potentially provide a long sought-after, safer and effective targeted therapy for kidney cancers, including metastatic renal cancer carcinomas.

### Administration and Pharmaceutical Formulations

In general, the compounds provided herein can be administered in a therapeutically effective amount by any of the accepted modes of administration for agents that serve similar utilities. By way of example, compounds provided herein may be administered orally, parenterally, transdermally, topically, rectally, or intranasally, or by way of intra-tumoral administration directly into a cancerous tumor. The actual amount of a compound provided herein, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

The data obtained from the cell culture assays and animal studies can be used in formulating a range of dosage for use in humans. The dosage of such compounds lies preferably within a range of circulating concentrations that include therapeutic efficacy with little or no toxicity. The dosage may vary within this range depending upon the dosage form employed and the route of administration utilized. For any compound used in the method provided herein, the therapeutically effective dose can be estimated initially from animal models. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans.

When employed as pharmaceuticals, the compounds provided herein are usually administered in the form of pharmaceutical compositions. These compounds can be administered by a variety of routes including oral, parenteral, transdermal, topical, rectal, and intranasal.

Compounds provided herein are effective as injectable, oral, inhalable, topical, or intra-tumor administration compositions. Such compositions are prepared in a manner well known in the pharmaceutical art and comprise at least one active compound.

This invention also includes pharmaceutical compositions which contain, as the active ingredient, one or more of the compounds provided herein above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions are preferably formulated in a unit dosage form, each dosage containing from about 0.1 to about 2000 mg, more usually about 1 to about 900 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound provided herein above is employed at no more than about 20 weight percent of the pharmaceutical composition, more preferably no more than about 15 weight percent, with the balance being pharmaceutically inert carrier(s).

An active compound is effective over a wide dosage range and is generally administered in a pharmaceutically or therapeutically effective amount. It, will be understood, however, that the amount of the compound actually administered can be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, compounds or pharmaceutical compositions thereof can be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 mg/kg to about 250 mg/kg, more preferably about 1.0 mg/kg to about 50 mg/kg of body weight/day.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills described herein may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions described herein may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Additionally, liposomal formulations of compounds of this invention may be used, for example, to enhance therapeutic effect against certain infections, such as pneumonia or ling infections.

Intra-tumoral administration of compounds provided herein employs solutions or gels thereof prepared in suitable aqueous solutions containing appropriate excipient additives, such as dextrose, polyethylene glycol, cremophore, cyclodextrin, and similar excipient additives.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

Other suitable formulations for use in the present invention can be found in Remington's Pharmaceutical Sciences, Mace Publishing Company, Philadelphia, Pa., 17th ed. (1985).

Optionally, the compounds of the present invention may be co-administered with additional agents, including antioxidants, such as ascorbic acid, or megalin-receptor inhibitors generally known to attenuate adverse effects of polymyxin drugs.

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference. Optionally, the compounds described herein could be administered as nanomicells, or nanomaterials-encapsulated compositions, prepared as described, for example, by Taki et al. in Pharmaceut., 2012, vol. 3, p. 1092.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

The disclosures of each and every patent, patent application and publication (for example, journals, articles and/or textbooks) cited herein are hereby incorporated by reference in their entirety. Also, as used herein and in the appended claims, singular articles such as “a”, “an” and “one” are intended to refer to singular or plural. While the present invention has been described herein in conjunction with a preferred aspect, a person with ordinary skills in the art, after reading the foregoing specification, can affect changes, substitutions of equivalents and other types of alterations to the invention as set forth herein. Each aspect described above can also have included or incorporated therewith such variations or aspects as disclosed in regard to any or all of the other aspects. The present invention is also not to be limited in terms of the particular aspects described herein, which are intended as single illustrations of individual aspects provided herein. Many modifications and variations of this invention can be made without departing from its spirit and scope, as will be apparent to those skilled in the art. Functionally equivalent methods within the scope of this invention, in addition to those enumerated herein, will be apparent to those skilled in the art from the foregoing descriptions. It is to be understood that this invention is not limited to particular methods, reagents, process conditions, materials and so forth, which can, of course, vary. It is also to be understood that the terminology used herein is for the purpose of describing particular aspects only, and is not intended to be limiting. Thus, it is intended that the specification be considered as exemplary.

1. A compound of the following formula I 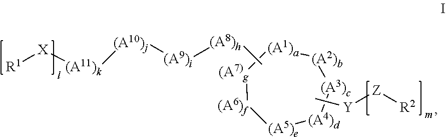
or a pharmaceutically acceptable salt, solvate, or hydrate thereof wherein:
R¹ and R² are optional groups, with at least one of the groups R¹ and R² being present in the formula I; and
R¹ and R² are independently selected from alkyl, aryl, biaryl, heteroaryl, heteroarylaryl, and arylheteroaryl; or
R¹ and R² are groups independently attached to X and Z, respectively, by subtracting a single or multiple H atom(s) from respective parent (precursor) structure(s) (H)ₙR and (H)ₒR² at any one of the following optional H-containing group(s) independently selected from NH, OH, SH, C(═O)OH, CONH, SO₂NH, and S(═O)NH when present in (H)ₙR and (H)ₒR²; and wherein
a) (H)ₙR and (H)ₒR² are independently compound(s) possessing biological or therapeutic activity; or
b) (H)ₙR and (H)ₒR² are independently a cytotoxic compound(s), an antibody(ies), or an immunomodulating compound(s) possessing an activity or capable of inducing an activity against one or more cancer cells; or
c) (H)ₙR¹ and (H)ₒR² are independently mono- or multi-valent antibody(ies) with activity against one or more cancer cells; or
d) (H)ₙR¹ and (H)ₒR² are independently afatinib ((E)-N-[4-(3-chloro-4-fluoroanilino)-7-[(3S)-oxolan-3-yl]oxyquinazolin-6-yl]-4-(dimethylamino)but-2-enamide), ARS-1630 ((R)-1-(4-(6-chloro-8-fluoro-7-(2-fluoro-6-hydroxyphenyl)quinazolin-4-yl)piperazin-1-yl)prop-2-en-1-one), axitinib (N-methyl-2-[[3-[(E)-2-pyridin-2-ylethenyl]-1H-indazol-6-yl]sulfanyl]benzamide), BGB-324 (1-(6,7-dihydro-5H-benzo[2,3]cyclohepta[2,4-d]pyridazin-3-yl)-3-N-[(7S)-7-pyrrolidin-1-yl-6,7,8,9-tetrahydro-5H-benzo[7]annulen-3-yl]-1,2,4-triazole-3,5-diamine), BLU-554 (N-[(3S,4S)-3-[[6-(2,6-dichloro-3,5-dimethoxyphenyl)quinazolin-2-yl]amino]oxan-4-yl]prop-2-enamide), brivanib ((S)—(R)-1-((4-((4-fluoro-2-methyl-1H-indol-5-yl)oxy)-5-methylpyrrolo[2,1-f][1,2,4]triazin-6-yl)oxy)propan-2-yl 2-aminopropanoate), (R)-1-((4-((4-fluoro-2-methyl-1H-indol-5-yl)oxy)-5-methylpyrrolo[2,1-f][1,2,4]triazin-6-yl)oxy)propan-2-ol, cabozantinib, cediranib, ceritinib, ciforadenant, derazantinib, dovitinib (4-amino-5-fluoro-3-(6-(4-methylpiperazin-1-yl)-1H-benzo[d]imidazol-2-yl)quinolin-2(1H)-one), E-7046 (4-[(1S)-1-[[3-(difluoromethyl)-1-methyl-5-[3-(trifluoromethyl)phenoxy]pyrazole-4-carbonyl]amino]ethyl]benzoic acid), emtansine, englerin ((1R,3aR,4S,5R,7R,8S,8aR)-5-(glycoloyloxy)-7-isopropyl-1,4-dimethyldecahydro-4,7-epoxyazulen-8-yl (2E)-3-phenylacrylate), foretinib, lenvatinib (4-[3-chloro-4-(cyclopropylcarbamoylamino)phenoxy]-7-methoxyquinoline-6-carboxamide), monomethyl auristatin E ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide), irinotecan, maytansinoid, neratinib, nilotinib, nintedanib, ozogamicin, paclitaxel, pazopanib (5-[[4-[(2,3-dimethylindazol-6-yl)-methylamino]pyrimidin-2-yl]amino]-2-methylbenzenesulfonamide), regorafenib, sacituzumab, selpercatinib, semaxanib ((Z)-3-((3,5-dimethyl-1H-pyrrol-2-yl)methylene)indolin-2-one), sorafenib (4-[4-[[4-chloro-3-(trifluoromethyl)phenyl]carbamoylamino]phenoxy]-N-methylpyridine-2-carboxamide), sunitinib ((Z)—N-(2-(diethylamino)ethyl)-5-((5-fluoro-2-oxoindolin-3-ylidene)methyl)-2,4-dimethyl-1H-pyrrole-3-carboxamide), SN38 (same 7-ethyl-10-hydroxy-camptothecin), trastuzumab, tesirine ([4-[[(2S)-2-[[(2S)-2-[3-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]propanoylamino]-3-methylbutanoyl]amino]propanoyl]amino]phenyl]methyl (6S,6aS)-3-[5-[[(6aS)-2-methoxy-8-methyl-11-oxo-6a,7-dihydropyrrolo[2,1-c][1,4]benzodiazepin-3-yl]oxy]pentoxy]-6-hydroxy-2-methoxy-8-methyl-11-oxo-6a,7-dihydro-6H-pyrrolo[2,1-c][1,4]benzodiazepine-5-carboxylate), temsirolimus ([(1R,2R,4S)-4-[(2R)-2-[(1R,9S,12S,15R,16E,18R,19R,21R,23S,24E,26E,28E,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl]3-hydroxy-2-(hydroxymethyl)-2-methylpropanoate), tivantinib, tivozanib (1-{2-chloro-4-[(6,7-dimethoxyquinolin-4-yl)oxy]phenyl}-3-(5-methylisoxazol-3-yl)urea), vatalanib, veliparib, or vinblastine; or variants of aforementioned structures derived from these by modification(s) of said structure(s); or
e) (H)ₙR¹ and (H)ₒR² are independently compound(s) active against a kidney cancer disease; or
f) (H)ₙR¹ is a heterocyclic structure(s) connected to X at one of heterocyclic nitrogen atom(s) present within the structure (H)ₙR¹; wherein said nitrogen atom becomes a nitrogen atom with a single positive charge, such as imidazolium, pyrazolium, pyridinium, or indazolium group; and
when an optional group R¹ is absent, then the fragment R¹X is replaced with R^{11a}, wherein R^{11a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), arylN(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
when an optional group R² is absent, then the fragment R²Z is replaced with R^{12a}, wherein R^{12a} is selected from H, Alk, C₃₋₇cycloalkyl, 5- to 6-membered heterocyclyl, aryl, biaryl, heteroaryl, AlkC(═O), AlkOC(═O), AlkNHC(═O), AlkN(C₁₋₁₂alkyl)C(═O), AlkSO₂, AlkNHSO₂, C₃₋₇cycloalkylC(═O), C₃₋₇cycloalkylOC(═O), C₃₋₇cycloalkylNHC(═O), C₃₋₇cycloalkylN(C₁₋₁₂alkyl)C(═O), arylC(═O), arylOC(═O), arylNHC(═O), arylN(C₁₋₁₂alkyl)C(═O), arylSO₂, arylNHSO₂, heteroarylC(═O), heteroarylOC(═O), heteroarylNHC(═O), heteroaryl N(C₁₋₁₂alkyl)C(═O), heteroarylSO₂, and heteroarylNHSO₂; or wherein
integers n and o are independently selected from 0, 1, 2, 3, 4, 5, 6, and 7, such that [n+o]≥1; and
A¹ through A¹¹ are optional amino acid residues independently selected from unsubstituted or substituted at any N atom alpha-, beta-, or gamma-amino acids, Ala, Arg, Asn, Asp, Cys, Glu, Gln, Gly, His, Ile, Leu, Lys, Met, Phe, Pro, Ser, L-homoserine, Thr, Trp, Tyr, Val, D-Ala, D-Arg, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid; and 5-amino-4-arylpentanoic acid;
A¹², A¹³, A¹⁴, and A¹⁵ are independently unsubstituted or substituted at any N atom alpha-, beta-, or gamma-amino acids, Ala, Arm, Asn, Asp, Cys, Glu, Gln, Gly, His, Ile, Leu, Lys, Met, Phe, Pro, Ser, L-homoserine, Thr, Trp, Tyr, Val, D-Ala, D-Arm, D-Asn, D-Asp, D-Cys, D-Glu, D-Gln, D-His, D-Ile, D-Leu, D-Lys, D-Met, D-Phe, D-Pro, D-Ser, D-homoserine, D-Thr, D-Trp, D-Tyr, D-Val, 3-aminoproline, 4-aminoproline, biphenylalanine (Bip), D-Bip, 2,3-diaminopropionic acid (Dap), 2,4-diaminobutyric acid (Dab), 2,5-diaminopentanoic acid, azetidine-2-carboxylic acid, azetidine-3-carboxylic acid, piperidine-2-carboxylic acid, 6-aminopiperidine-2-carboxylic acid, 5-aminopiperidine-2-carboxylic acid, 4-aminopiperidine-2-carboxylic acid, 3-aminopiperidine-2-carboxylic acid, piperidine-3-carboxylic acid, 6-aminopiperidine-3-carboxylic acid, 5-aminopiperidine-3-carboxylic acid, 4-aminopiperidine-3-carboxylic acid, piperazine-2-carboxylic acid, 6-aminopiperazine-2-carboxylic acid, 8-azabicyclo[3.2.1]octane-2-carboxylic acid, 4-amino-8-azabicyclo[3.2.1]octane-2-carboxylic acid, 3-amino-8-azabicyclo[3.2.1]octane-2-carboxylic acid, 6-azabicyclo[3.1.1]heptane-2-carboxylic acid, 3-amino-6-azabicyclo[3.1.1]heptane-2-carboxylic acid, and 4-amino-6-azabicyclo[3.1.1]heptane-2-carboxylic acid, 4-amino-3-arylbutanoic acid, 4-amino-3-(3-chlorophenyl)butanoic acid, or 5-amino-4-arylpentanoic acid, or similar natural or unnatural amino acid residues;
integers a through m are independently selected from 0, 1, and 2, and wherein
[m+l]≥1, wherein the symbol “l” in [m+l] and at the group [R¹—X]ₗ represents a letter “l”; and wherein
when any of integers a through k is 0, then any of the two groups adjacent to a respective absent group (according to the integer 0 at said absent group) are connected to each other directly; and wherein
when the integers a through g are all 0, then the groups A¹-A⁷ are absent, and the group A⁸ terminates with either COOH, CH₂OH, or C(═O)NR³R⁴, wherein R³ and R⁴ are independently selected from H, alkyl, aryl, heteroaryl, and heterocyclyl; or the group A⁸ is directly connected to the group Y; and
each optional divalent group X is independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R^{5a}), N(R^{5a})C(═O)(C₁₋₁₂alkylene),
(C₁₋₁₂alkylene)N(R^{5a})C(═O), C(═O)N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O), C(═O)CR⁵═CR⁷—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH](CR⁵R⁶)ₚ(CR⁷R⁸)ᵣC(═O)O(CR⁹R¹⁰)ₛC(═O),
C(═O)N[CH₂CH₂NHC(═O)CH₂CH₂CH(NH₂)COOH](CR⁵R⁶)ₚ(CR⁷R⁸)ᵣ OC(═O)
(CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ
(CR⁹R¹⁰)ₛC(═O),
C(═O)N[CH₂CH₂NHC(═O)CH₂CH₂CH(NH₂)COOH](CR⁵R⁶)ₚ OC(═O)(CR⁷R⁸)ᵣ
(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O),
or any variant of above groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5a})SO₂ therein;
R⁶, R⁷, R⁸, R⁹, and R¹⁰ are independently H, NH, halo, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl;
R⁵ is H, NH₂, NH(C₁₋₆alkyl), NH(OC₁₋₆alkyl), C₁₋₁₄alkyl, C₃₋₆cycloalkyl, aryl, arylalkyl, biaryl, biarylalkyl, or heteroarylalkyl;
R^{5a} is H, C₁₋₁₄alkyl, C₃₋₆cycloalkyl, aryl, arylalkyl, biaryl, biarylalkyl, or heteroarylalkyl; or wherein
any two of R⁵ through R¹⁰, together with the atom(s) to which they are attached form a 4 to 7-member saturated or unsaturated heterocycle containing at least one O atom, or containing one O atom and an additional heteroatom independently selected from N and S and wherein remaining atoms are carbon; or wherein
any two of R⁵ through R¹⁰, together with the carbon atom(s) to which they are attached form a 4 to 7-member saturated or unsaturated C₃₋₆cycloalkylene; or any of i) ii) R⁶ and R⁷, ii) R⁵ and R⁶, and iii) R⁹ and R¹⁰, together with the atom to which they are attached form a saturated or unsaturated C₃₋₆cycloalkylene; or wherein
any two of R⁵ through R¹⁰ together with the atom(s) to which they are attached form a 5 to 7-member saturated or unsaturated heterocycle wherein the ring optionally comprises an additional heteroatom selected from N, O, and S, and wherein the remaining atoms are carbon; or the resulting ring comprises 1,3-dioxol-2-one heterocycle; or wherein
R⁶ and R⁸ together with the atom to which they are attached form a 4 to 6-member saturated heterocycle containing at least one O atom wherein the heterocycle optionally comprises an additional heteroatom selected from N, O, and S, and wherein the remaining atoms are carbon; or the resulting ring comprises 1,3-dioxol-2-one heterocycle; and wherein
integers p, r, and s are independently selected from 0, 1, and 2; and wherein when fragments (CR⁵R⁶)ₚ(CR⁷R⁸)ᵣ(CR⁹R¹⁰)ₛ or (OCR⁵R⁶)ₚ(CR⁷R⁸)ᵣ(CR⁹R¹⁰)ₛ are present, then [p+r+s]≥1; and wherein
when fragments (CR⁵R⁶)ₚ(CR⁷R⁸)ᵣ or (OCR⁵R⁶)ₚ(CR⁷R⁸)ᵣ are present, then [p+r]≥1; and wherein
when fragments (CR⁷R⁸)ᵣ(CR⁹R¹⁰)ₛ or (OCR⁷R⁸)ᵣ(CR⁹R¹⁰)ₛ are present, then [r+s]≥1; or
alternatively, each optional divalent group X is independently comprised of the following structures, optionally connected to one to two amino acid residue(s) A¹² or A¹³:
(C₁₋₁₂alkylene)OC(═O), OC(═O)(C₁₋₂alkylene),
(C₁₋₁₂alkylene)OC(═O), C(═O)O(C₁₋₁₂alkylene),
(C₁₋₁₂alkylene)C(═O),
N(R^{5a})C(═O)(C₁₋₁₂alkylene),
(C₁₋₁₂alkylene)N(R^{5a})C(═O), C(═O)N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC(═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚ(CR⁷R⁸)ᵣ(CR⁹R¹⁰)ᵣN(R^{5a})C(═O),
C(═O)CR⁵═CR⁷—(CR⁹R¹⁰)ₚC(═O),
C(═O)OCR⁵═CR⁷—(CR⁹R¹⁰)ₚC(═O),
C(═O)N(R^{5a})CR⁵═CR⁷—(CR⁹R¹⁰)ᵣC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₛC(═O),
C(═O)OCR⁵═CR⁷—(CR⁹R¹⁰)ₚC(═O),
C(═O)N(R^{5a})CR⁵═CR⁷—(CR⁹R¹⁰)ᵣC(═O),
C(═O)CR⁵═CR⁷—(CR⁹R¹⁰)ₚOC(═O),
C(═O)CR⁵═CR⁷—(CR⁹R¹⁰)ᵣN(R^{5a})C(═O),
or any variant of the above X groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5s})SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
additionally, each optional divalent group X independently incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₙO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), and N(C₁₋₁₄alkyl)(CH₂)ₚO(CH₂)ᵣO(CH₂)ₛN(C₁₋₁₄alkyl)C(═O);
optional divalent groups Y and Z are independently selected from O, NH, N(C₁₋₆alkyl), S, S—S, S—N, S(═O), SO₂, C(═O), OC(═O), C(═O)O, NHC(═O)NH, N(C₁₋₆alkyl)C(═O)NH, N(C₁₋₆ alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R^{5a}), N(R^{5a})C(═O)(C₁₋₁₂alkylene),
(C₁₋₁₂alkylene)N(R^{5a})C(═O), C(═O)N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O)(CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)—(CR⁹R¹⁰)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O)
C(═O)N[CH₂CH₂OC(═O)CH₂CH₂CH(NH₂)COOH]CH₂CH₂C(═O),C(═O)N[CH₂CH₂NHC(═O)
CH₂CH₂CH(NH₂)COOH](CR⁵R⁶)ₚ(CR⁷R⁸)ᵣC(═O)O(CR⁹R¹⁰)ₛC(═),C(═O)N[CH₂CH₂NHC(═O)
CH₂CH₂CH(NH₂)COOH](CR⁵R⁶)ₚ(CR⁷R⁸)ᵣ OC(═O) (CR⁹R¹⁰)ₛC(═O),
C(═O)N[CH₂CH₂NHC(═O)CH₂CH₂CH(NH₂)COOH](CR⁵R⁶)ₚC(═O)O(CR⁷R⁸)ᵣ
(CR⁹R¹⁰)ₛC(═O),
C(═O)N[CH₂CH₂NHC(═O)CH₂CH₂CH(NH₂)COOH](CR′R⁶)ₚOC(═O)(CR⁷R⁸)ᵣ
(CR⁹R¹⁰)ₛC(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)-CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O), or
any variant of above groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5a})SO₂ therein; or
alternatively, the optional group Z is comprised of the following structures, optionally connected to one to two amino acid residue(s) A¹² or A¹³:
(C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O),
N(R^{5a})C(═O)(C₁₋₁₂alkylene),
(C₁₋₁₂alkylene)N(R^{5a})C(═O), C(═O)N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O), C(═O)CR⁵═CR⁷—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₚC(═O),
C(═O)OCR⁵═CR⁷—(CR⁹R¹⁰)ₚC(═O),
C(═O)N(R^{5a})CR⁵═CR⁷—(CR⁹R¹⁰)ᵣC(═O),
C(═O)CR⁵═CR⁷—(CR⁹R¹⁰)ₚOC(═O),
C(═O)CR⁵═CR⁷—(CR⁹R¹⁰)ᵣN(R^{5a})C(═O); or any variant of the above Z groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5a})SO₂ therein; and wherein when both amino acid residues A¹² and A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A⁴, A⁵, A⁶, A⁷ or A⁸.

2. The compound of claim 1 of formula I: 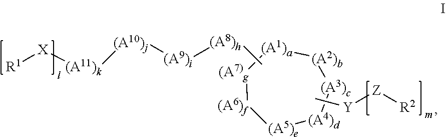
or a pharmaceutically acceptable salt, solvate, or hydrate thereof wherein:
optional divalent groups X, Y, and Z are independently selected from O, NIH, N(C₁₋₆alkyl), S, S—S, S—N, S(═O), SO₂, C(═O), OC(═O), C(═O)O, NHC(═O)NIH, N(C₁₋₆alkyl)C(═O)NH, N(C₁₋₆alkyl)C(═O)NC₁₋₆alkyl), NHC(═O)NC₁₋₆alkyl), C₁₋₁₂alkylene, arylene, biarylene, (heteroaryl)arylene, (aryl)heteroarylene, heterocyclylene,
(C₁₋₁₂alkylene)C(═O)O, OC(═O)(C₁₋₁₂alkylene),
(C₁₋₁₂alkylene)OC(═O), C(═O)O(C₁₋₁₂alkylene),
(C₁₋₁₂alkylene)C(═O)N(R^{5a}), N(R^{5a})C(═O)(C₁₋₁₂alkylene),
(C₁₋₁₂alkylene)N(R^{5a})C(═O), C(═O)N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)N(R^{5a})(CR⁵R⁶)ₚ(CR⁷R⁸)ᵣ(CR⁹R¹⁰)ₛC(═O),
C(═O)CR⁵═CR⁷—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C(═O),
C(═O)(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O), and
any variant of above groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5a})SO₂ therein;
X is a group comprised of the following structures, additionally connected to one to two amino acid residue(s) A¹² or A¹³, at the right side of the following structures:
(C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O),
N(R^{5a})C(═O)(C₁₋₁₂alkylene),
(C₁₋₁₂alkylene)N(R^{5a})C(═O), C(═O) N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC═O),
C(═O)(CR⁵R⁶)ₚ(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O); or any variant of these groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5a})SO₂ therein; and wherein when both amino acid residues A¹² and A¹³ are incorporated at the right side of above groups to comprise the group X, then residues A¹² or A¹³ are interconnected with a peptide bond A¹²-A¹³; and wherein
when an optional group X is absent, then group R¹ is directly connected to one of groups A⁸, A⁹, A¹⁰, or A¹¹; or
Z is a group comprised of the following structures, additionally connected to one to two amino acid residue(s) A¹² or A¹³, at the left side of the following structures: (C₁₋₁₂alkylene)OC(═O), OC(═O)(C₁₋₁₂alkylene),
(C₁₋₁₂alkylene)OC(═O), C(═O)O(C₁₋₁₂alkylene),
(C₁₋₁₂alkylene)C(═O),
N(R^{5a})C(═O)(C₁₋₁₂alkylene),
(C₁₋₁₂alkylene)N(R^{5a})C(═O), C(═O) N(R^{5a})(C₁₋₁₂alkylene),
C(═O)(CR⁵R⁶)ₚ(CR⁷R⁸)ᵣ(CR⁹R¹⁰)ₛC(═O),
C(═O)O(CR⁵R⁶)O(CR⁷R⁸)ᵣ(CR⁹R¹⁰)ₛC(═O),
P(═O)(OCR⁵R⁶)ₚ(CR⁷R⁸)ᵣ(CR⁹R¹⁰)ₛC═O),
C(═O)(CR⁷R⁸)ₚ(CR⁹R¹⁰)ᵣP(═O)(OCR⁵R⁶)ₘ,
P(═O)(NHCR⁵R⁶)ₚ(CR⁷R⁸)ᵣ(CR⁹R¹⁰)ₛC(═O),
P(═O)(OCR⁵R⁶)CF₂, P(═O)(OCR⁵R⁶)CF₂(CR⁷R⁸)ᵣC(═O),
P(═O)(OH)CF₂, P(═O)(OH)CF₂(CR⁷R⁸)ᵣC(═O),
C(═O)(CR⁷R⁸)ᵣ(CR⁹R¹⁰)ₛP(═O)(NHCR⁵R⁶)ₚ,
C(═O)O(CR⁵R⁶)ₚ(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR^{S}═CR⁷—(CR⁹R¹⁰)ₛOC(═O),
C(═O)CR⁵═CR⁷—(CR⁹R¹⁰)ₛN(R^{5a})C(═O),
C(═O)N(R^{5a})SO₂(CR⁵R⁶)ₚ(CR⁷R⁸)ᵣ(CR⁹R¹⁰)ₛC(═O),
C(═O)(CR⁵R⁶)ₚ(CR⁷R⁸)ᵣ(CR⁹R¹⁰)ₛN(R^{5a})SO₂C(═O),
C(═O)(CR⁵R⁶)ₚS—S(CR⁷R⁸)ᵣ(CR⁹R¹⁰)ₛC(═O),
C(═O)O(CR⁵R⁶)ₚS—S(CR⁷R⁸)ᵣ(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣ(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)(CR⁵R⁶)ₚ(CR⁷R⁸)ᵣS—S(CR⁹R¹⁰)ₛC(═O),
C(═O)O(CR⁵R⁶)ₚ(CR⁷R⁸)ᵣS—S(CR⁹R¹⁰)ₛC(═O),
C(═O)N(R^{5a})(CR⁵R⁶)ₚ(CR⁷R⁸)ᵣ(CR⁹R¹⁰)ₛC(═O),
C(═O)(CR⁵R⁶)ₚS—S(CR⁷R⁸)ᵣS—S(CR⁹R¹⁰)ₛOC(═O),
C(═O)(CR⁵R⁶)ₚ(CR⁷R⁸)ᵣ(CR⁹R¹⁰)ₛN(R^{5a})C(═O),
C(═O)CR⁵═CR⁷—S—S—(CR⁹R¹⁰)ₛC(═O),
C(═O)OCR⁵═CR⁷—(CR⁹R¹⁰)ₛC(═O),
C(═O)N(R^{5a})CR⁵═CR⁷—(CR⁹R¹⁰)ₛC(═O),
C(═O)CR⁵═CR⁷—(CR⁹R¹⁰)ₛOC(═O),
C(═O)CR⁵═CR⁷—(CR⁹R¹⁰)ₛN(R^{5a})C(═O); or any variant of these groups formed by repositioning(s), addition(s), or deletion(s) of the fragments C(═O), OC(═O), N(R^{5a})C(═O), P(═O)(OCR⁵R⁶)CF₂, P(═O)(OH)CF₂, or C(═O)N(R^{5a})SO₂ therein; and wherein when both amino acid residues A¹² and A¹³ are incorporated at the left side of above groups to comprise the group Z, then residues A¹² or A¹³ are interconnected with a peptide bond A¹²-A¹³; and
when an optional group Z is absent, then the group R² is directly connected to one of groups Y, A¹, A², A³, A⁴, A⁵, A⁶, A⁷ or A⁸; or wherein
an optional group X, either at its left or right side therein, incorporates additional divalent groups selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alkyl)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)O(CH₂)ᵣO(CH₂)ₛN(C₁₋₁₄alkyl)C(═O), N(C₁₋₁₄alkyl)(CH₂)O(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), and N(C₁₋₁₄alkyl)(CH₂)ₚO(CH₂)ᵣO(CH₂)ₛN(C₁₋₁₄alkyl)C(═O).

3. The compound of claim 1 according to formula I, or a pharmaceutically acceptable salt, solvate, or hydrate thereof, wherein
the integers a through g are all equal to 1;
A¹ is Thr or Ser;
A², A³ A⁶, and A⁷ are independently selected from Dab, Dap, Ser, and Thr;
A⁴ is Leu or Ile; and
A⁵ is Phe, D-Phe, Bip, D-Bip, Val, or D-Val.

4. The compound of claim 1 according to formula I, or a pharmaceutically acceptable salt, solvate, or hydrate thereof, wherein
the cyclic peptide structure comprised of optional amino acid residues A¹ through A⁷ is a cyclic peptide structure identical to that present in polymyxin A, polymyxin B, polymyxin B nonapeptide (H-Thr-Dab-cyclo[Dab-Dab-D-Phe-Leu-Dab-Dab-Thr]), polymyxin B heptapeptide (H-cyclo[Dab-Dab-D-Phe-Leu-Dab-Dab-Thr]), polymyxin E, or octapeptin.

5. The compound of claim 1 according to formula II: 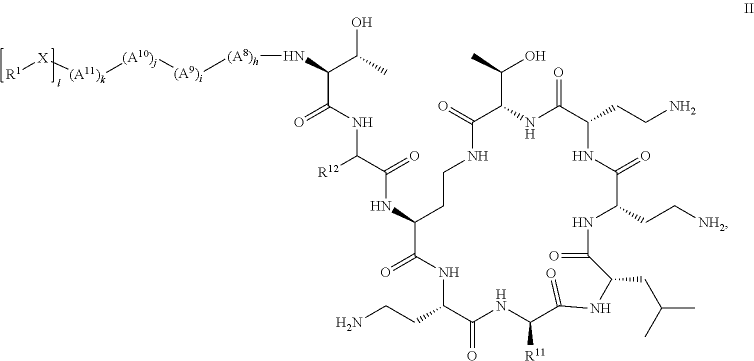
or a pharmaceutically acceptable salt, solvate, or hydrate thereof, wherein:
R¹¹ is CH₂CH(CH₃)₂ or CH₂Ph; and
R¹² is CH₂NH₂ or CH₂CH₂NH₂.

6. The compound of formula II of claim 5, or a pharmaceutically acceptable salt, solvate, or hydrate thereof, wherein each X in formula II is independently selected from the structures below, wherein either the left side or the right side of X depicted below is connected to R¹:

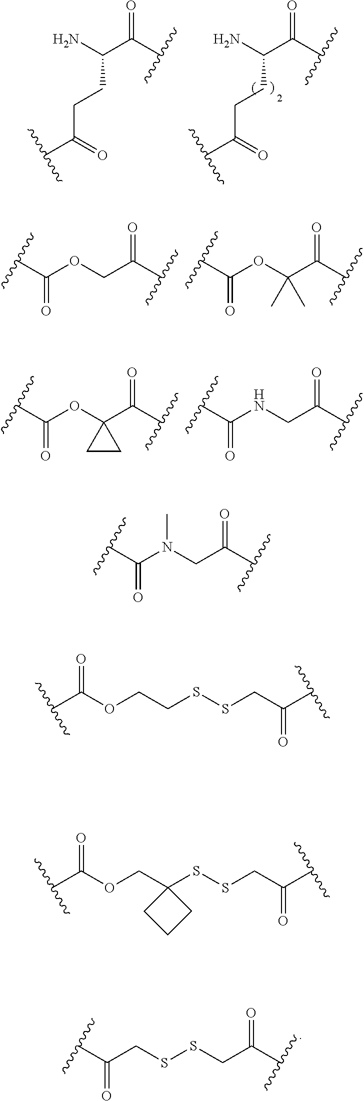

7. The compound of formula II of claim 5, or a pharmaceutically acceptable salt, solvate, or hydrate thereof, wherein each X in formula II is independently selected from the following structures, connected to R¹ at the left side or the right side of X below:

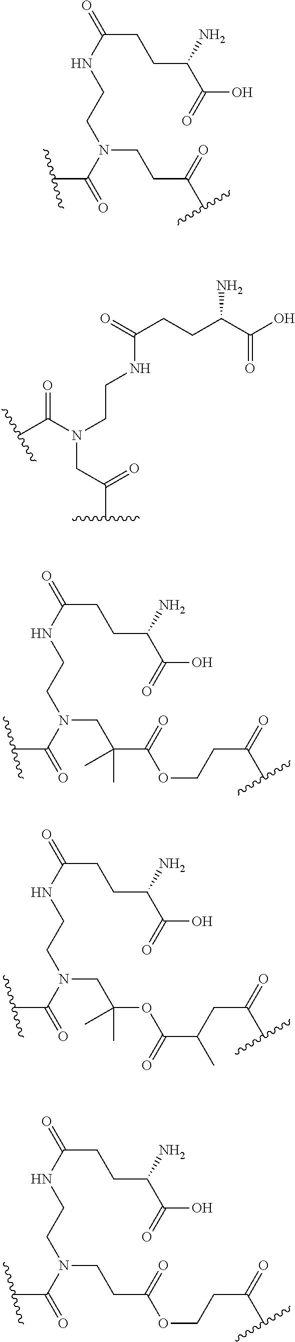 [SMILES: *C(=O)CCN(CCNC(=O)CC[C@H](N)C(=O)O)C(*)=O ; *C(=O)CN(CCNC(=O)CC[C@H](N)C(=O)O)C(*)=O ; *C(=O)CCOC(=O)C(C)(C)CN(CCNC(=O)CC[C@H](N)C(=O)O)C(*)=O ; *C(=O)CC(C)C(=O)OC(C)(C)CN(CCNC(=O)CC[C@H](N)C(=O)O)C(*)=O ; *C(=O)CCOC(=O)CCN(CCNC(=O)CC[C@H](N)C(=O)O)C(*)=O]

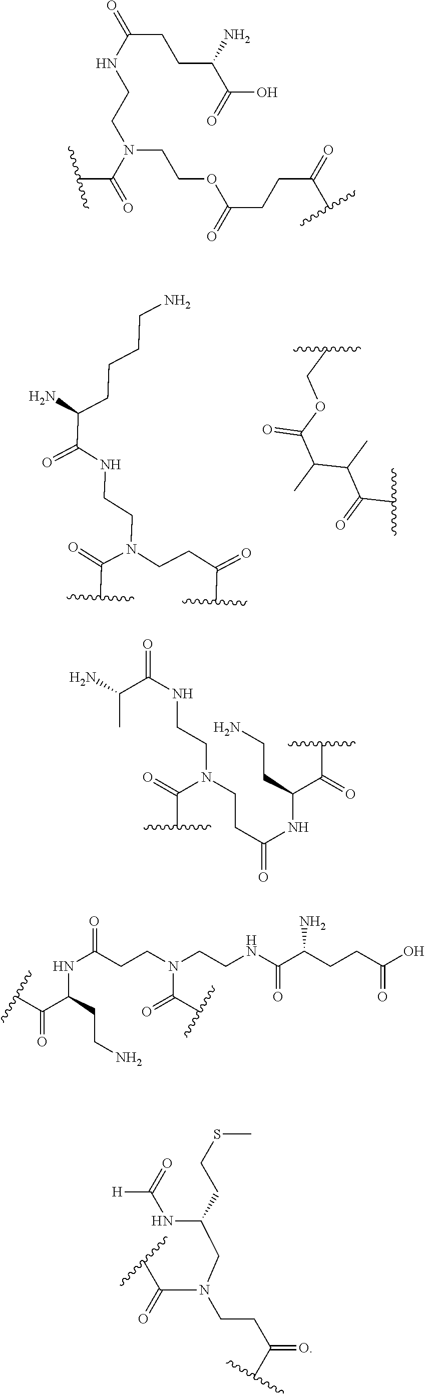 [SMILES: *C(=O)CCC(=O)OCCN(CCNC(=O)CC[C@H](N)C(=O)O)C(*)=O ; *C(=O)CCN(CCNC(=O)[C@@H](N)CCCCN)C(*)=O ; *COC(=O)C(C)C(C)C(*)=O ; *C(=O)[C@H](CCN)NC(=O)CCN(CCNC(=O)[C@H](C)N)C(*)=O ; *C(=O)[C@H](CCN)NC(=O)CCN(CCNC(=O)[C@H](N)CCC(=O)O)C(*)=O ; *C(=O)CCN(C[C@@H](CCSC)NC=O)C(*)=O]

8. The compound of formula II of claim 5, or a pharmaceutically acceptable salt, solvate, or hydrate thereof, wherein each X, either at its left or right side, independently incorporates additional divalent group selected from C₁₋₁₂alkylene, C₂₋₁₂alkenylene, C₂₋₁₂alkynylene, (CH₂)ₚO(CH₂)ᵣO(CH₂)ₛC(═O), (CH₂)ₚO(CH₂)ᵣO(CH₂)ₛOC(═O), (CH₂)ₚO(CH₂)ᵣO(CH₂)ₛNHC(═O), (CH₂)ₚO(CH₂)ᵣO(CH₂)ₛN(C₁₋₁₄alky)C(═O), O(CH₂)ₚO(CH₂)ᵣO(CH₂)ₛC(═O), O(CH₂)ₚO(CH₂)ᵣO(CH₂)ₛOC(═O), O(CH₂)ₚO(CH₂)ᵣO(CH₂)ₛNHC(═O), O(CH₂)ₚO(CH₂)ᵣO(CH₂)ₛN(C₁₋₁₄alkyl)C(═O), NH(CH₂)ₚO(CH₂)ᵣO(CH₂)ₛC(═O), NH(CH₂)ₚO(CH₂)ᵣO(CH₂)ₛOC(═O), NH(CH₂)ₚO(CH₂)ᵣO(CH₂)ₛNHC(═O), NH(CH₂)ₚO(CH₂)ᵣO(CH₂)ₛN(C₁₋₁₄alkyl)C(═O), N(C₁₋₁₄alkyl)(CH₂)ₚO(CH₂)ᵣO(CH₂)ₛC(═O), N(C₁₋₁₄alkyl)(CH₂)ₚO(CH₂)ᵣO(CH₂)ₛOC(═O), N(C₁₋₁₄alkyl)(CH₂)ₚO(CH₂)ᵣO(CH₂)ₛNHC(═O), and N(C₁₋₁₄alkyl)(CH₂)ₚO(CH₂)ᵣO(CH₂)ₛN(C₁₋₁₄alkyl)C(═O).

9. The compound of claim 1 according to formula III 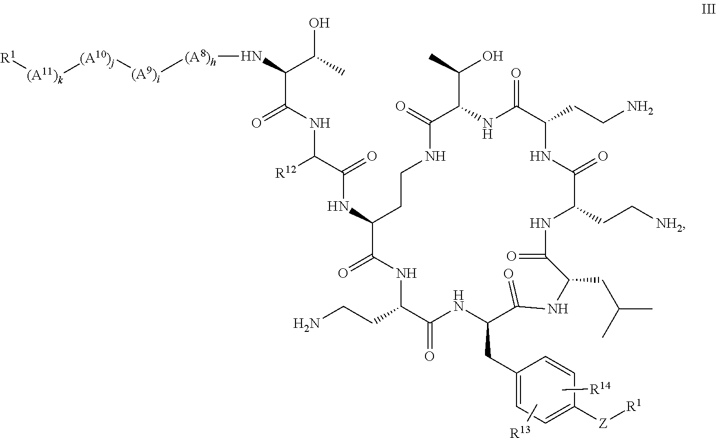
or a pharmaceutically acceptable salt, solvate, or hydrate thereof, wherein
R¹³ and R¹⁴ are independently selected from H, halo, NH₂, CN, OH, OC₁₋₁₄alkyl, Oaryl, NH(C₁₋₆alkyl), NH(OC₁₋₆alkyl), C₁₋₁₄alkyl, C₃₋₆cycloalkyl, aryl, arylalkyl, biaryl, biarylalkyl, heteroarylalkyl, C(═O)OH, C₁₋₁₄alkylC(═O)OH, and C₁₋₁₄alkylC(═O)—OC₁₋₁₄alkyl.

10. The compound of the formula III of claim 9, wherein Z in formula III is selected from the structures below, wherein the right side of Z is connected to R²:

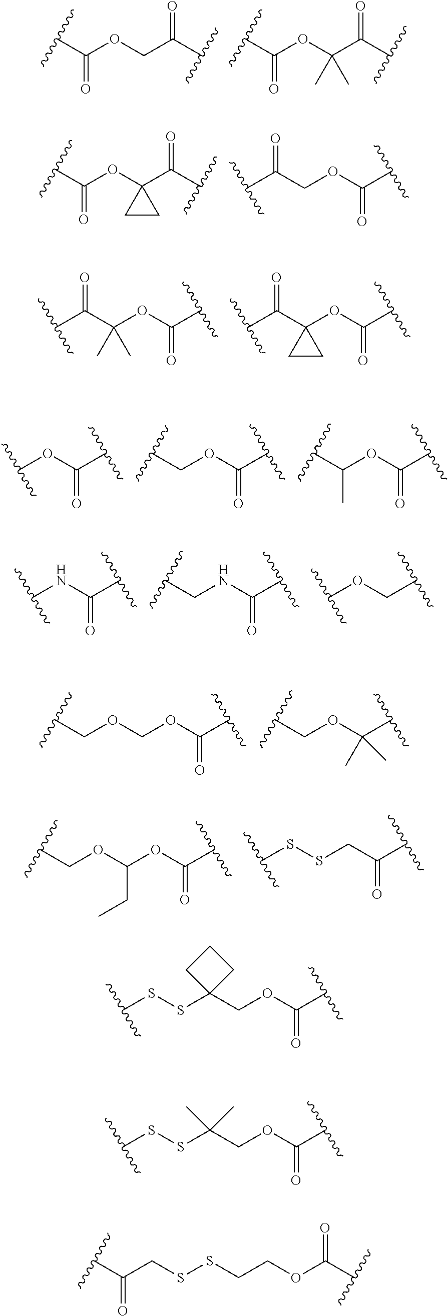

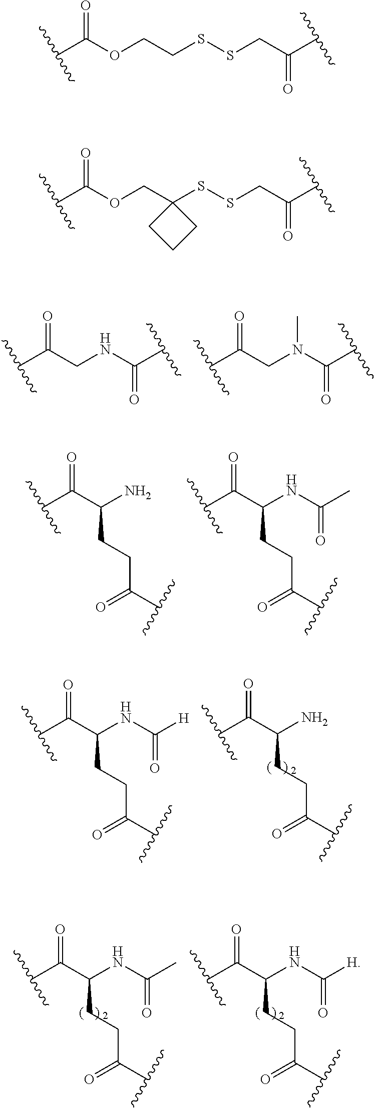

11. The compound of claim 1 according to formula IV 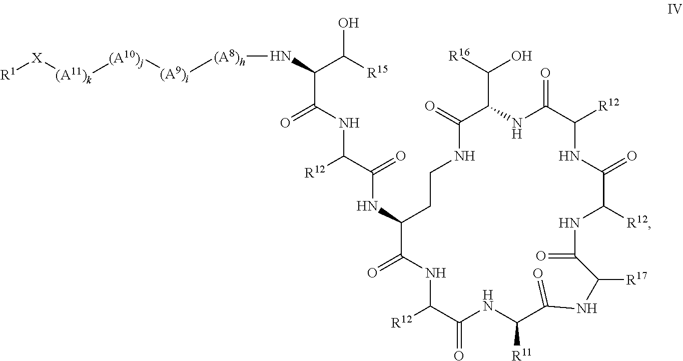
or a pharmaceutically acceptable salt, solvate, or hydrate thereof, wherein
X is selected from the following structures and is connected to R¹ at the left side of X:
C(═O)N[(CR⁵R⁶)ₚ(CR⁷R⁸)ᵣNHC(═O)A¹⁴](CR⁹R¹⁰)ₛC(═O),
C(═O)N[(CR⁵R⁶)ₚ(CR⁷R⁸)ᵣNHC(═O)A¹⁴](CR⁹R¹⁰)ₛNHC(═O),
C(═O)N[(CR⁵R⁶)ₚ(CR⁷R⁸)ᵣNHC(═O)A¹⁴](CR⁹R¹⁰)ₛOC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛC(═O),
C(═O)N[(CR⁵R⁶)ₚ(CR⁷R⁸)ᵣN(C₁₋₆alkyl)C(═O)A¹⁴](CR⁹R¹⁰)ₛNHC(═O),
C(═O)N[(CR⁵R⁶)ₚ(CR⁷R⁸)ᵣN(C₁₋₆alkyl)C(═O)A¹⁴](CR⁹R¹⁰)ₛOC(═O),
C(═O)N[(CR⁵R⁶)ₚ(CR⁷R⁸)ᵣOC(═O)A¹⁴](CR⁹R¹⁰)ₛC(═O),
C(═O)N[(CR⁵R⁶)ₚ(CR⁷R⁸)ᵣOC(═O)A¹⁴](CR⁹R¹⁰)ₛN(C═O),
C(═O)N[(CR⁵R⁶)ₚ(CR⁷R⁸)ᵣOC(═O)A¹⁴](CR⁹R¹⁰)ₛO(C═O),
C(═O)N[(CR⁵R⁶)ₚ(CR⁷R⁸)ᵣNHC(═O)A¹⁴A¹⁵](CR⁹R¹⁰)ₛC(═O),
C(═O)N[(CR⁵R⁶)ₚ(CR⁷R⁸)ᵣNHC(═O)A¹⁴A¹⁵](CR⁹R¹⁰)ₛN(C═O),
C(═O)N[(CR⁵R⁶)ₚ(CR⁷R⁸)ᵣNHC(═O)A¹⁴A¹⁵](CR⁹R¹⁰)ₛO(C═O),
C(═O)N[(CR⁵R⁶)ₚ(CR⁷R⁸)ᵣOC(═O)A¹⁴A¹⁵](CR⁹R¹⁰)ₛC(═O),
C(═O)N[(CR⁵R⁶)ₚ(CR⁷R⁸)ᵣOC(═O)A¹⁴A¹⁵](CR⁹R¹⁰)ₛN(C═O),
C(═O)N[(CR⁵R⁶)ₚ(CR⁷R⁸)ᵣOC(═O)A¹⁴A¹⁵](CR⁹R¹⁰)ₛO(C═O),
C(═O)N[(CR⁵R⁶)ₚ(CR⁷R⁸)ᵣNHC(═O) (CR⁹R¹⁰)ₛNCH(NH₂)COOH]CH₂CH₂C(═O),
C(═O)N[(CR⁵R⁶)ₚ(CR⁷R⁸)ᵣN(C₁₋₆alkyl)C(═O)(CR⁹R¹⁰)ₛNCH(NH₂)COOH]CH₂CH₂C(═O),
C(═O)N[CH₂CH₂NHC(═O)CH₂CH₂CH(NH₂)COOH]CH₂CH₂C(═O),
C(═O)N[CH₂CH₂NHC(═O)CH₂CH₂CH(NH₂)COOC₁₋₆alkyl]CH₂CH₂C(═O),
C(═O)N[CH₂CH₂OC(═O)CH₂CH₂CH(NH₂)COOH]CH₂CH₂C(═O),
C(═O)N[CH₂CH₂NHC(═O)CH₂CH₂CH(NH₂)COOH]CH₂CH₂C(═O)OCH₂CH₂C(═O),
C(═O)N[CH₂CH₂NHC(═O)CH₂CH₂CH(NH₂)COOH]CH₂CH₂OC(═O)CH₂CH₂C(═O),
C(═O)N[CH₂CH₂NHC(═O)CH₂CH₂CH(NH₂)COOH]CH₂CMe₂C(═O)OCH₂CH₂C(═O),
C(═O)N[CH₂CH₂NHC(═O)CH₂CH₂CH(NH₂)COOH]CH₂CH₂OC(═O)CH(Me)CH₂C(═O),
C(═O)N[CH₂CH₂N(C₁₋₆alkyl)C(═O)CH₂CH₂CH(NH₂)COOH]CH₂CH₂C(═O),
(S)—C(═O)N[CH₂CH₂NHC(═O)CH₂CH₂CH(NH₂)COOH]CH₂CH₂C(═O),
(R)—C(═O)N[CH₂CH₂NHC(═O)CH₂CH₂CH(NH₂)COOH]CH₂CH₂C(═O),
(S)—C(═O)N[CH₂CH₂NHC(═O)CH₂CH₂CH(NH(C═O)R⁷)COOH]CH₂CH₂C(═O),
(R)—C(═O)N[CH₂CH₂NHC(═O)CH₂CH₂CH(NH(C═O)R⁷)COOH]CH₂CH₂C(═O),
(S)—C(═O)N[CH₂CH₂NHC(═O)CHNH(Me)]CH₂CH₂C(═O),
(R)—C(═O)N[CH₂CH₂NHC(═O)CHNH(Me)]CH₂CH₂C(═O),
(S)—C(═O)N[CH₂CH₂NHC(═O)CHNH(CH₂CH₂CH₂CH₂NH₂)]CH₂CH₂C(═O),
(R)—C(═O)N[CH₂CH₂NHC(═O)CHNH(CH₂CH₂CH₂CH₂NH₂)]CH₂CH₂C(═O),
(S)—C(═O)N[CH₂CH₂NHC(═O)CHNH(CH₂CH₂NH₂)]CH₂CH₂C(═O), and
(R)—C(═O)N[CH₂CH₂NHC(═O)CHNH(CH₂CH₂NH₂)]CH₂CH₂C(═O);
R¹¹ is C₁₋₁₂alkyl, CH(CH₃)₂, CH₂aryl, or CH₂Ph;
R¹² is CH₂NH₂, CH₂CH₂NH₂, or CH₂CH₂CH₂CH₂NH₂; and
R¹⁵, R¹⁷ and R¹⁷ are independently H, Me, or C₁₋₁₂alkyl.

12. The compound of the claim 11 of formula IV, or a pharmaceutically acceptable salt, solvate, or hydrate thereof, wherein R¹ is selected from the structures below:

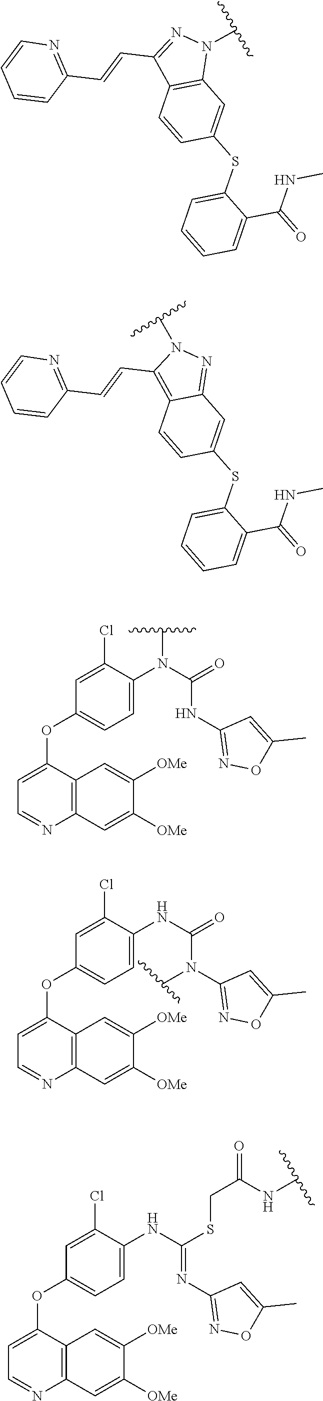 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1 ; *N(C(=O)Nc1cc(C)on1)c1ccc(Oc2ccnc3cc(OC)c(OC)cc23)cc1Cl ; *N(C(=O)Nc1ccc(Oc2ccnc3cc(OC)c(OC)cc23)cc1Cl)c1cc(C)on1 ; *NC(=O)CS/C(=N\c1cc(C)on1)Nc1ccc(Oc2ccnc3cc(OC)c(OC)cc23)cc1Cl]

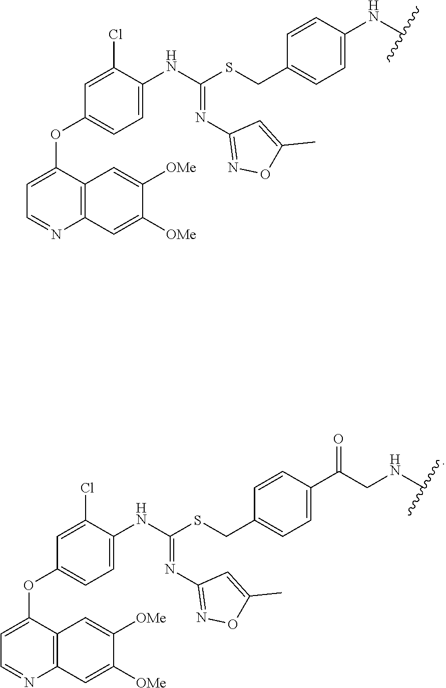 [SMILES: *Nc1ccc(CS/C(=N\c2cc(C)on2)Nc2ccc(Oc3ccnc4cc(OC)c(OC)cc34)cc2Cl)cc1 ; *NCC(=O)c1ccc(CS/C(=N\c2cc(C)on2)Nc2ccc(Oc3ccnc4cc(OC)c(OC)cc34)cc2Cl)cc1]

13. The compound of claim 1 or a pharmaceutically acceptable salt, solvate, or hydrate thereof, according to formula V: 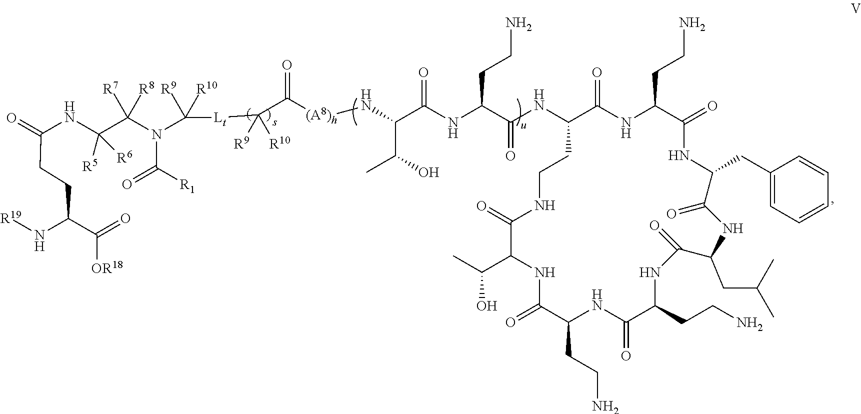
or a pharmaceutically acceptable salt, solvate, or hydrate thereof, wherein
R¹⁸ is H or C₁₋₁₂alkyl;
R¹⁹ is H, C₁₋₁₂alkyl, C(═O)C₁₋₁₂alkyl, C(═O)OC₁₋₁₂alkyl, C(═O)OC₁₋₁₂alkyl, C(═O)NHC₁₋₁₂alkyl, SO₂C₁₋₁₂alkyl, SO₂aryl, C(═O)C₃₋₇cycloalkyl, C(═O)OC₃₋₇cycloalkyl, C(═O)NHC₃₋₇cycloalkyl, C(═O)NHC₁₋₁₂alkyl, SO₂C₃₋₇cycloalkyl;
each optional group L is selected from alkyl, CR²⁰R²¹OC(═O)CR²²R²³ and CR²⁰R²¹C(═O)CR²²R²³;
R²⁰ through R²³ are independently selected from H, C₁₋₁₂alkyl, and C₃₋₇cycloalkyl; or any of the two adjacent groups R²⁰ and R²¹ or R²² and R²³ independently taken together form a C₃₋₇cycloalkyl group; and
integer t is 0, 1, or 2; and
integer u is 0 or 1.

14. The compound of claim 1, or a pharmaceutically acceptable salt, solvate, or hydrate thereof, wherein R¹ is selected from structures below:

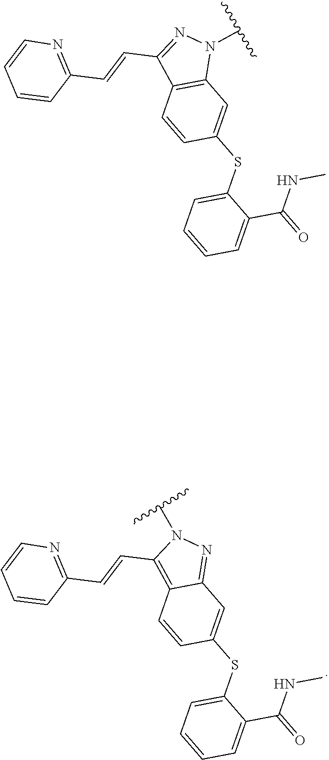 [SMILES: *n1nc(/C=C/c2ccccn2)c2ccc(Sc3ccccc3C(=O)NC)cc21 ; *n1nc2cc(Sc3ccccc3C(=O)NC)ccc2c1/C=C/c1ccccn1]

15. The compound of claim 1 selected from the following structures, or a pharmaceutically acceptable salt, solvate, or hydrate thereof, wherein, if present, PMBN group is a polymyxin B nonapeptide (H-Thr-Dab-cyclo[Dab-Dab-D-Phe-Leu-Dab-Dab-Thr]) residue incorporated into structure(s) below with a chemical bond formed through the replacement of the H atom at the H-Thr amino acid group and Dab is 2,4-diaminobutyric acid:

| Cpd. | |
|---|---|
| No. | Structure |
| 1 | 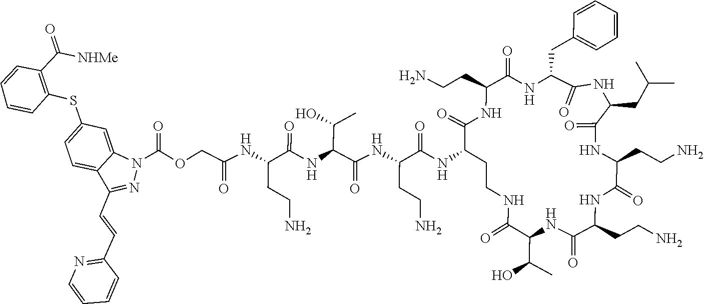 [SMILES: CNC(=O)c1ccccc1Sc1ccc2c(/C=C/c3ccccn3)nn(C(=O)OCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 2 | 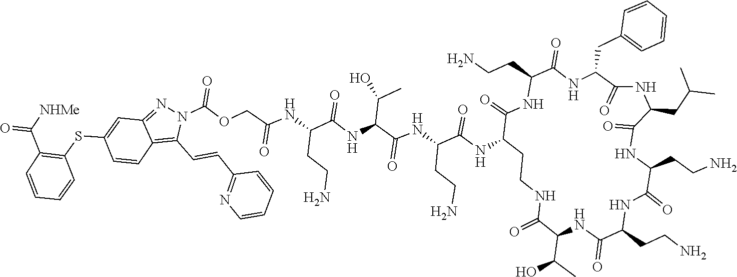 [SMILES: CNC(=O)c1ccccc1Sc1ccc2c(/C=C/c3ccccn3)n(C(=O)OCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)nc2c1] |
| 3 | 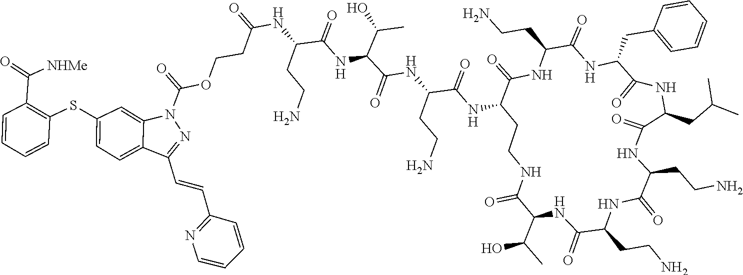 [SMILES: CNC(=O)c1ccccc1Sc1ccc2c(/C=C/c3ccccn3)nn(C(=O)OCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 4 | 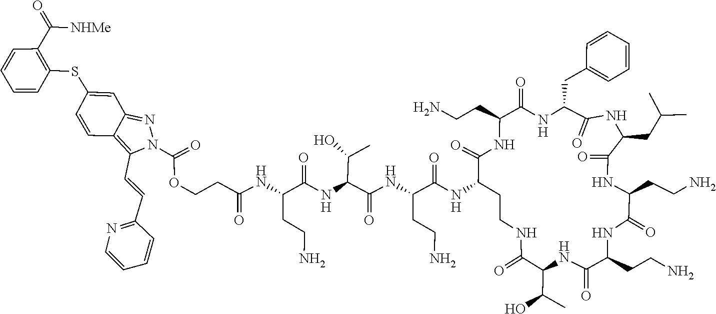 [SMILES: CNC(=O)c1ccccc1Sc1ccc2c(/C=C/c3ccccn3)n(C(=O)OCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)nc2c1] |
| 5 | 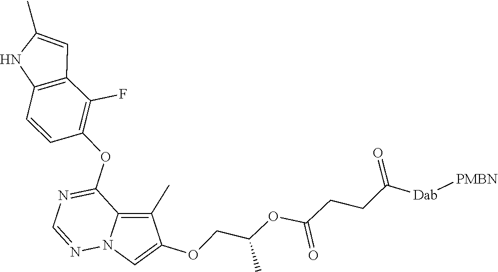 |
| 6 | 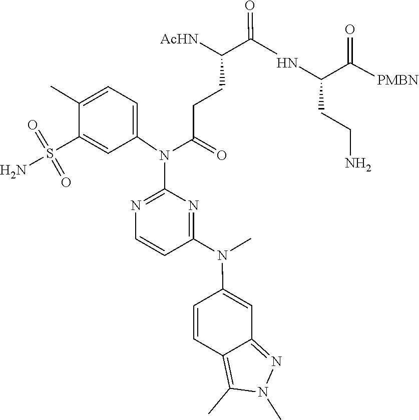 |
| 7 | 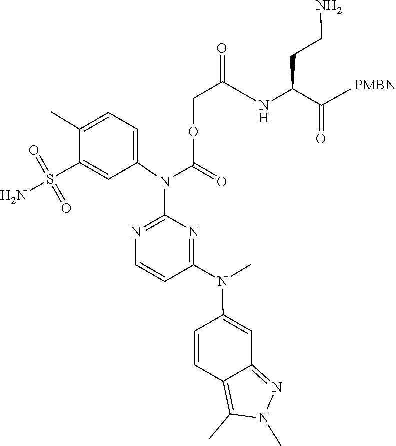 |
| 8 | 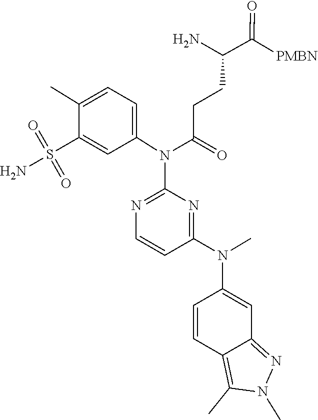 |
| 9 | 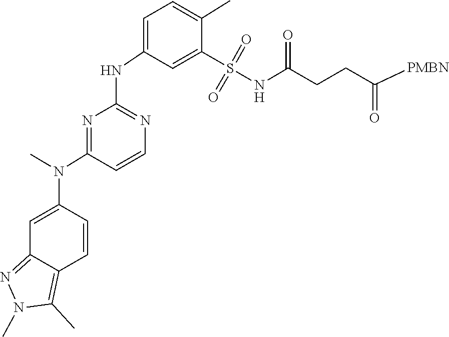 |
| 10 | 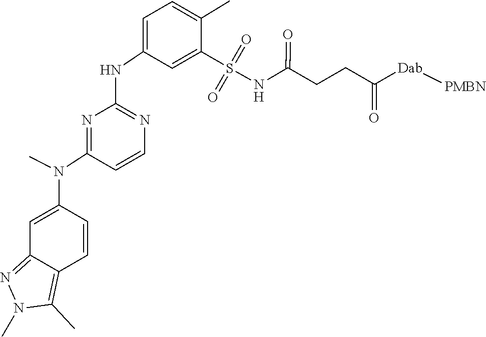 |
| 11 | 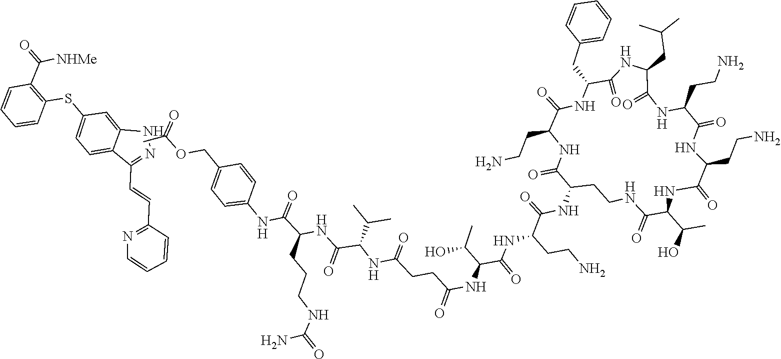 |
| 12 | 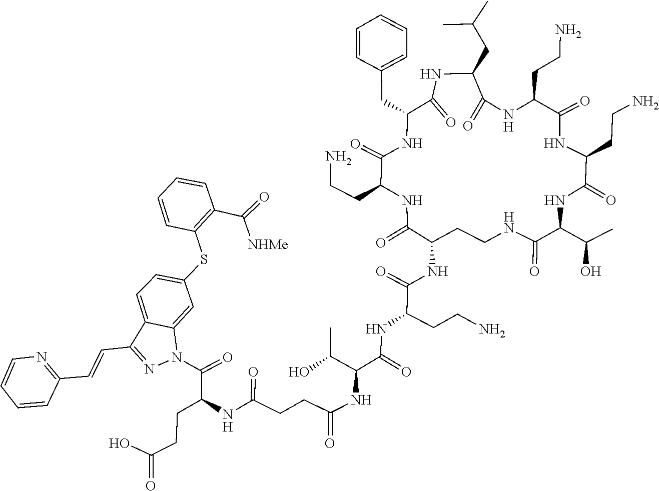 [SMILES: CNC(=O)c1ccccc1Sc1ccc2c(/C=C/c3ccccn3)nn(C(=O)[C@H](CCC(=O)O)NC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 13 | 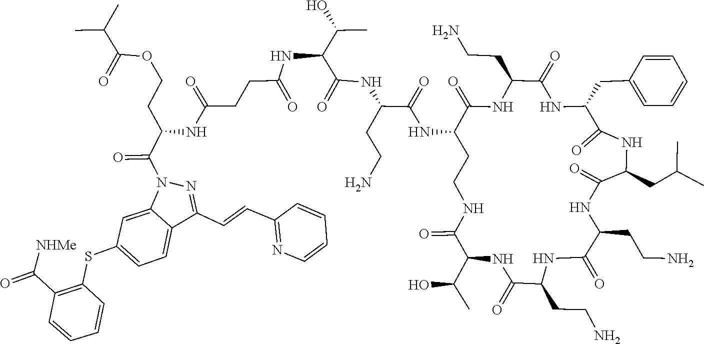 [SMILES: CNC(=O)c1ccccc1Sc1ccc2c(/C=C/c3ccccn3)nn(C(=O)[C@H](CCOC(=O)C(C)C)NC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 14 | 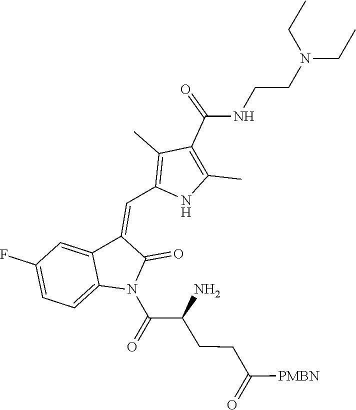 |
| 15 | 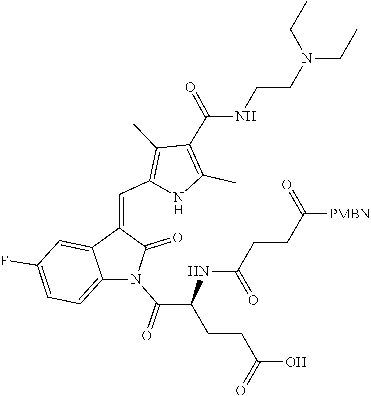 |
| 16 | 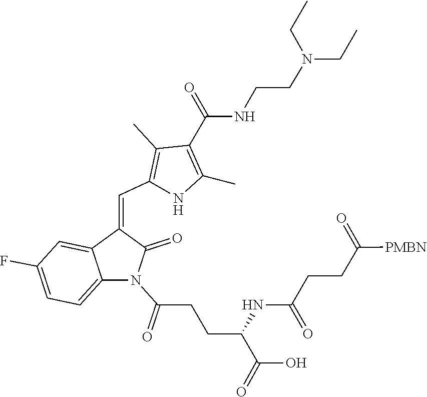 |
| 18 | 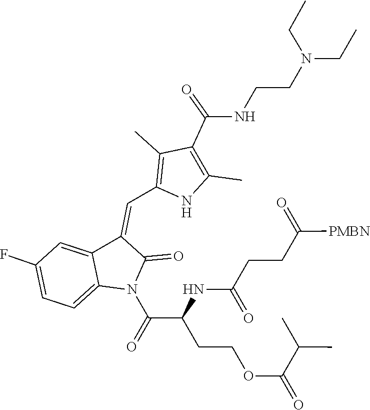 |
| 19 | 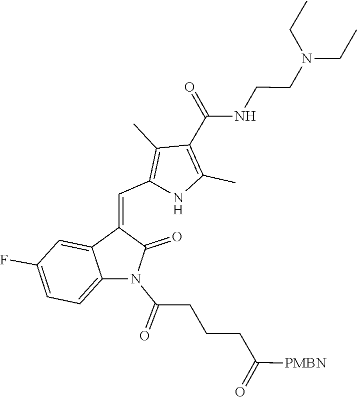 |
| 20 | 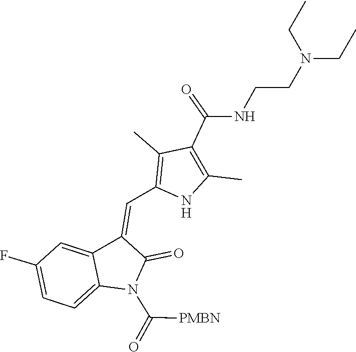 |
| 21 | 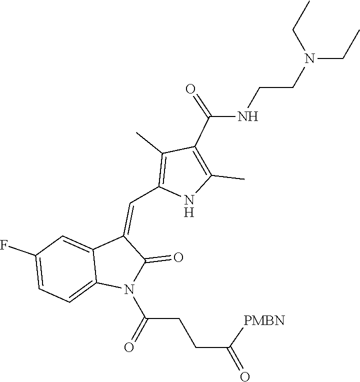 |
| 22 | 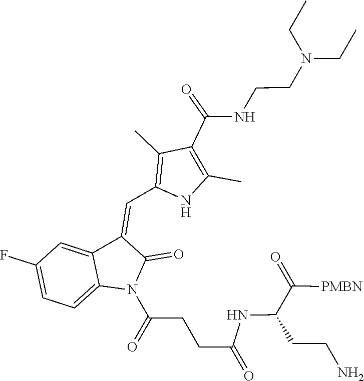 |
| 23 | 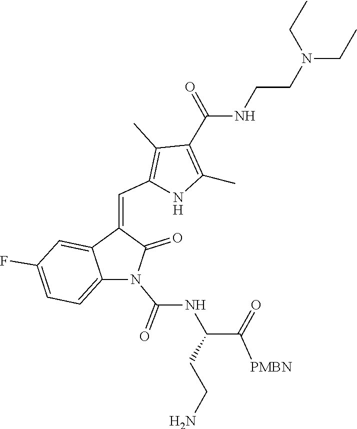 |
| 24 | 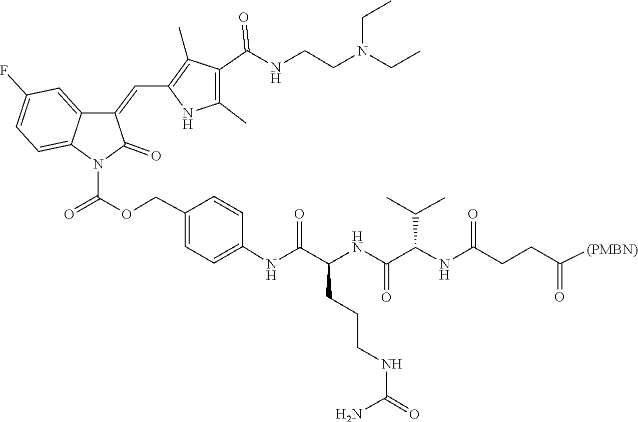 |
| 25 | 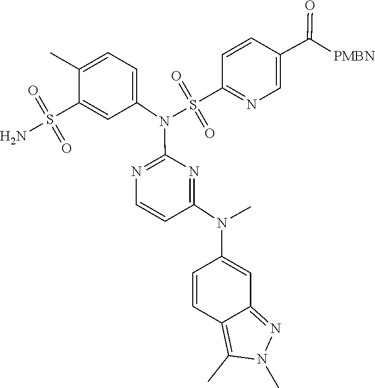 |
| 26 | 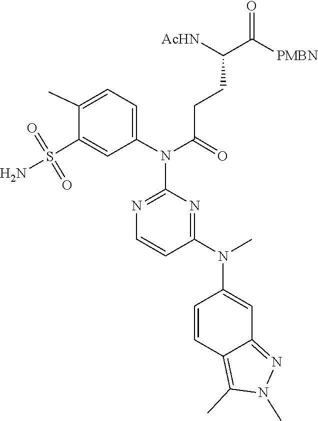 |
| 27 | 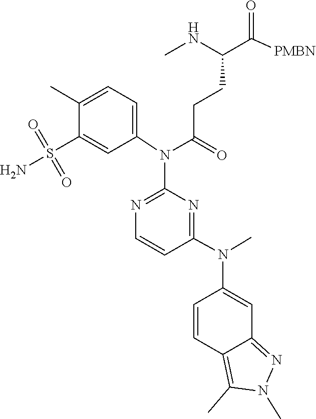 [SMILES: CN[C@H](C=O)CCC(=O)N(c1ccc(C)c(S(N)(=O)=O)c1)c1nccc(N(C)c2ccc3c(C)n(C)nc3c2)n1 ; "PMBN"] |
| 28 | 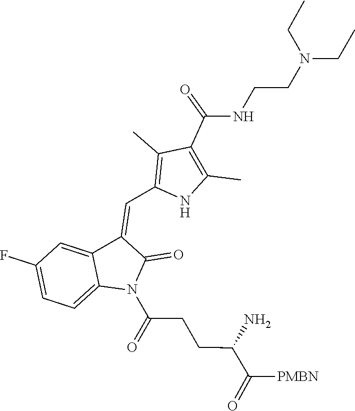 |
| 29 | 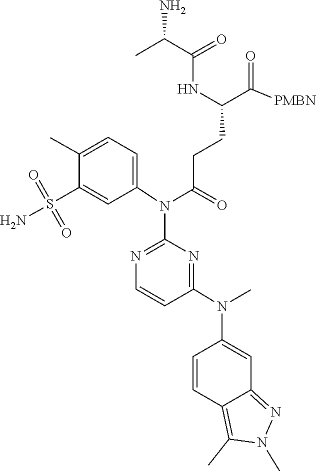 |
| 30 | 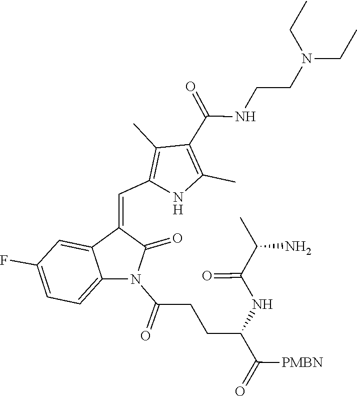 |
| 31 | 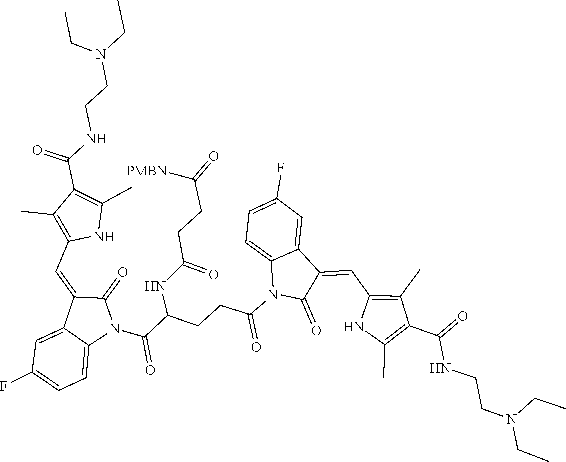 |
| 32 | 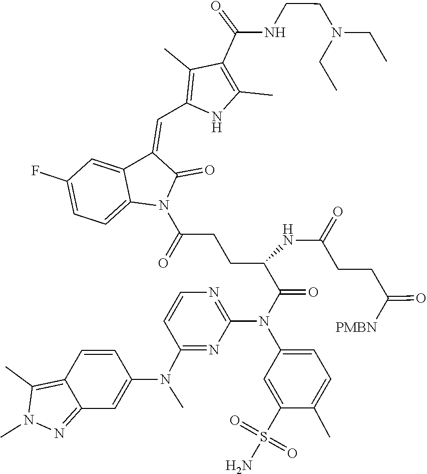 |
| 33 | 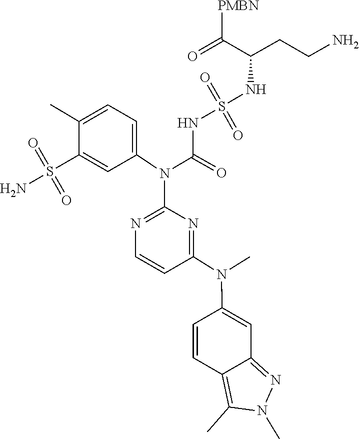 [SMILES: "PMBN" ; Cc1ccc(N(C(=O)NS(=O)(=O)N[C@H](C=O)CCN)c2nccc(N(C)c3ccc4c(C)n(C)nc4c3)n2)cc1S(N)(=O)=O] |
| 34 | 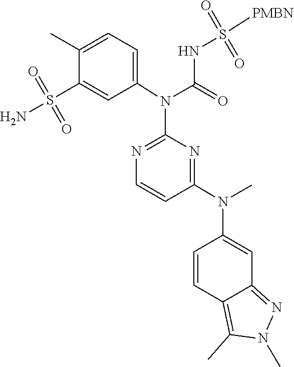 |
| 35 | 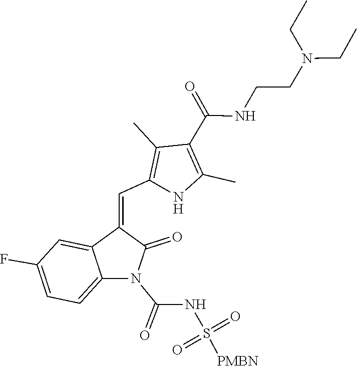 |
| 36 | 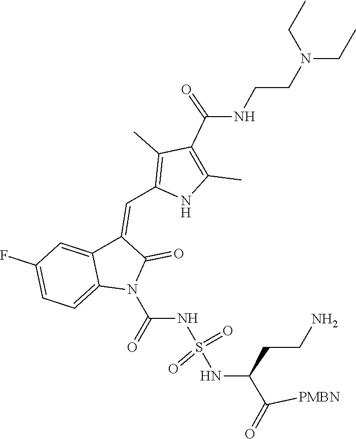 |
| 37 | 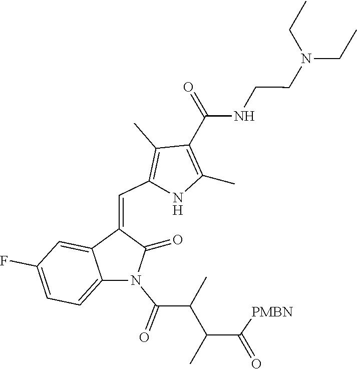 |
| 38 | 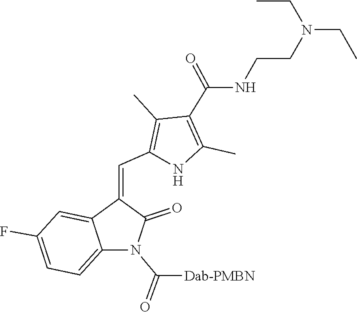 |
| 39 | 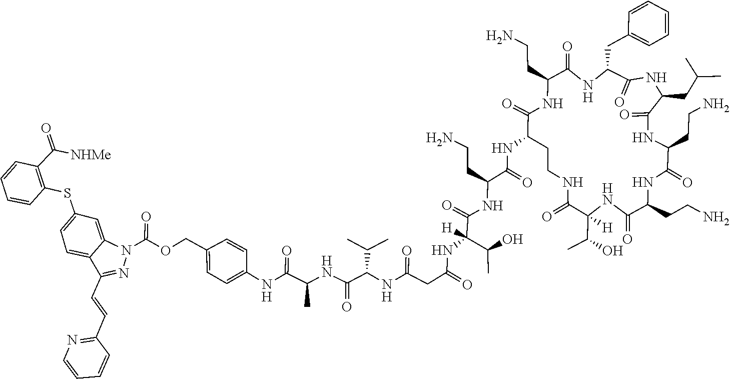 [SMILES: CNC(=O)c1ccccc1Sc1ccc2c(/C=C/c3ccccn3)nn(C(=O)OCc3ccc(NC(=O)[C@H](C)NC(=O)[C@@H](NC(=O)CC(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@H](C)O)C(C)C)cc3)c2c1] |
| 40 | 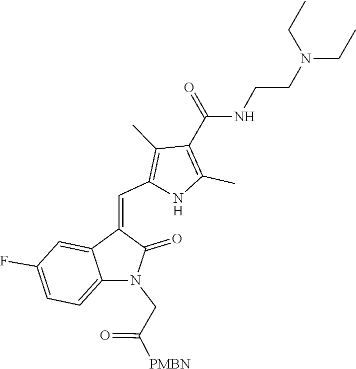 |
| 41 | 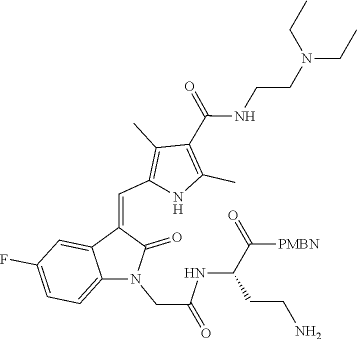 |
| 42 | 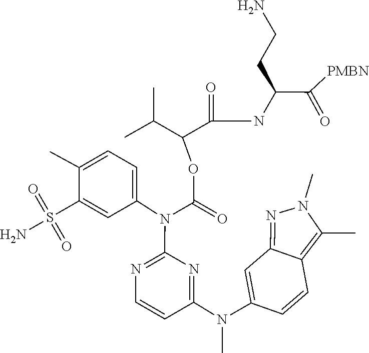 |
| 43 | 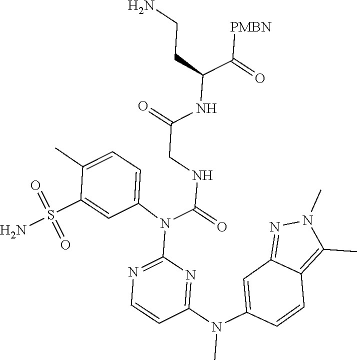 |
| 44 | 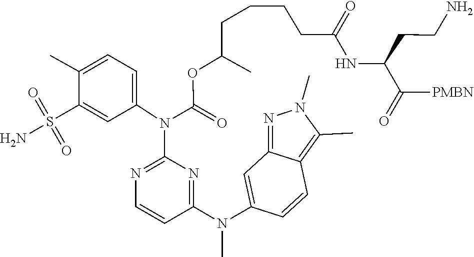 |
| 45 | 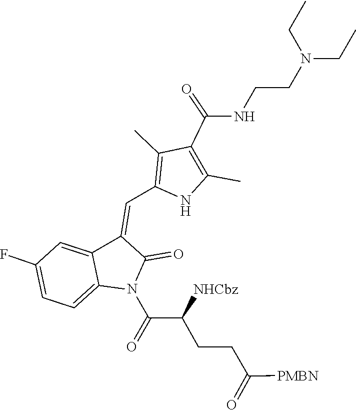 |
| 46 | 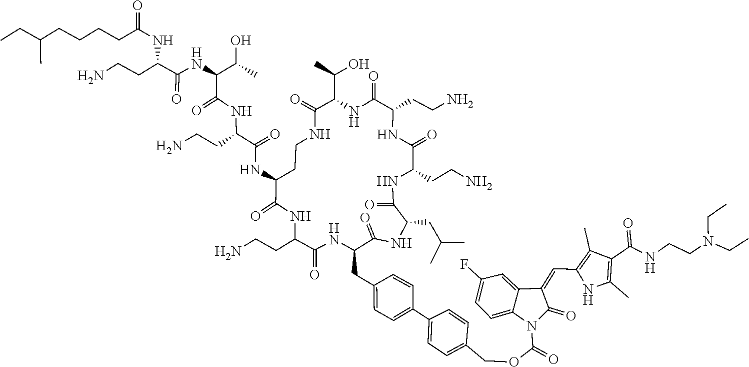 [SMILES: CCC(C)CCCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccc(-c3ccc(COC(=O)N4C(=O)/C(=C\c5[nH]c(C)c(C(=O)NCCN(CC)CC)c5C)c5cc(F)ccc54)cc3)cc2)NC(=O)C(CCN)NC1=O)[C@@H](C)O] |
| 47 | 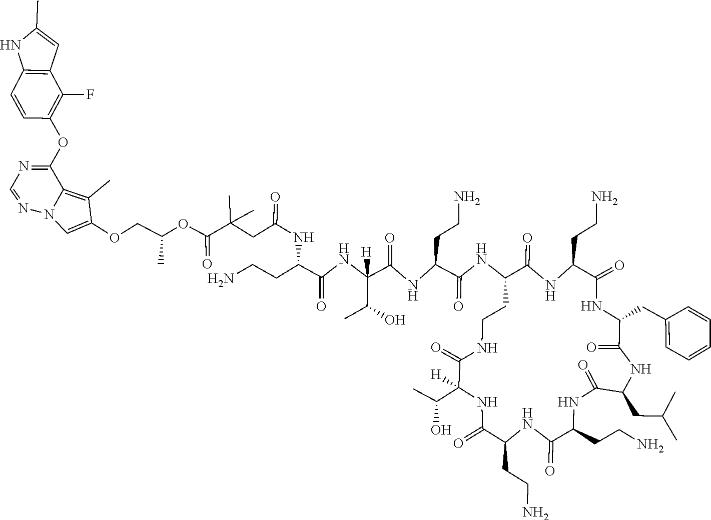 [SMILES: Cc1cc2c(F)c(Oc3ncnn4cc(OC[C@@H](C)OC(=O)C(C)(C)CC(=O)N[C@@H](CCN)C(=O)N[C@H](C(=O)N[C@@H](CCN)C(=O)N[C@H]5CCNC(=O)[C@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@@H](C)O)c(C)c34)ccc2[nH]1] |
| 48 | 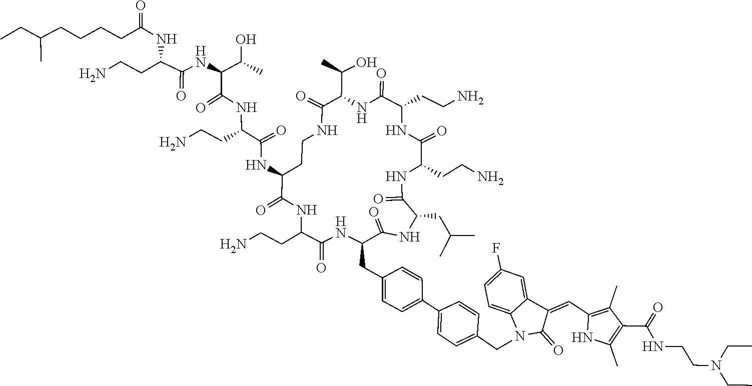 [SMILES: CCC(C)CCCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccc(-c3ccc(CN4C(=O)/C(=C\c5[nH]c(C)c(C(=O)NCCN(CC)CC)c5C)c5cc(F)ccc54)cc3)cc2)NC(=O)C(CCN)NC1=O)[C@@H](C)O] |
| 49 | 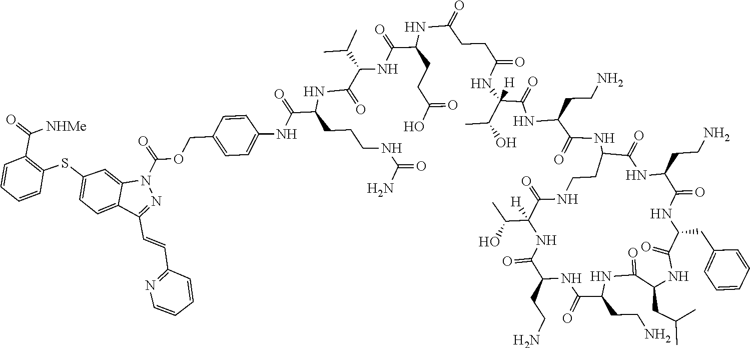 [SMILES: CNC(=O)c1ccccc1Sc1ccc2c(/C=C/c3ccccn3)nn(C(=O)OCc3ccc(NC(=O)[C@H](CCCNC(N)=O)NC(=O)[C@@H](NC(=O)[C@H](CCC(=O)O)NC(=O)CCC(=O)N[C@H](C(=O)N[C@@H](CCN)C(=O)NC4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)C(C)C)cc3)c2c1] |
| 50 | 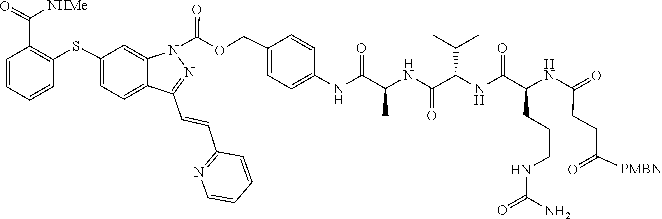 |
| 51 | 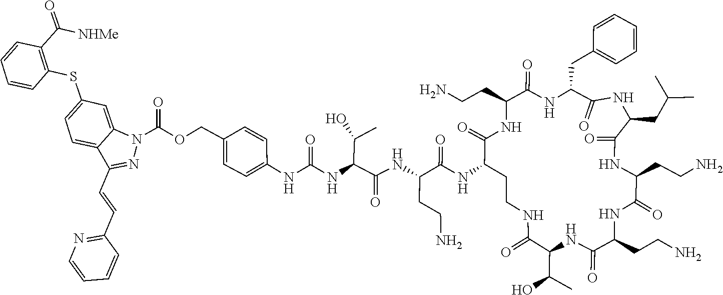 [SMILES: CNC(=O)c1ccccc1Sc1ccc2c(/C=C/c3ccccn3)nn(C(=O)OCc3ccc(NC(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c2c1] |
| 52 | 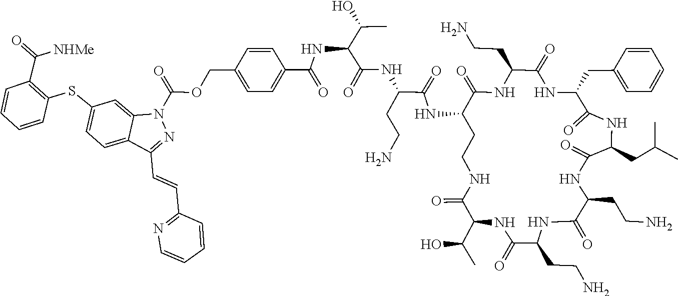 [SMILES: CNC(=O)c1ccccc1Sc1ccc2c(/C=C/c3ccccn3)nn(C(=O)OCc3ccc(C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c2c1] |
| 53 | 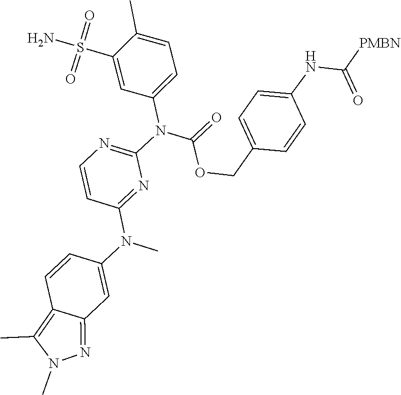 |
| 54 | 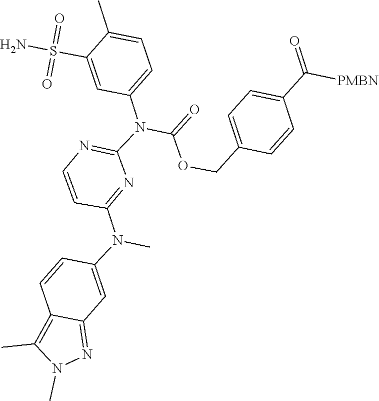 [SMILES: Cc1ccc(N(C(=O)OCc2ccc(C=O)cc2)c2nccc(N(C)c3ccc4c(C)n(C)nc4c3)n2)cc1S(N)(=O)=O ; "PMBN"] |
| 55 | 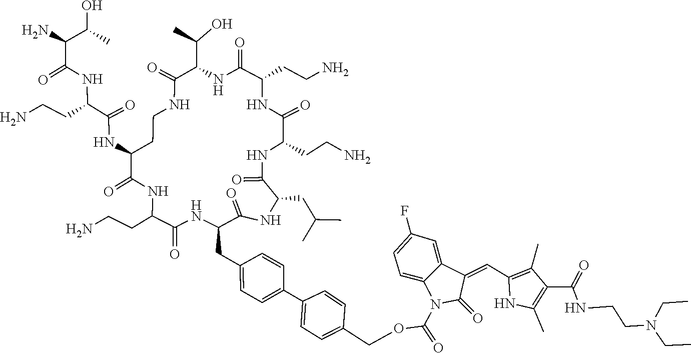 [SMILES: CCN(CC)CCNC(=O)c1c(C)[nH]c(/C=C2\C(=O)N(C(=O)OCc3ccc(-c4ccc(C[C@H]5NC(=O)C(CCN)NC(=O)[C@@H](NC(=O)[C@H](CCN)NC(=O)[C@@H](N)[C@@H](C)O)CCNC(=O)[C@H]([C@@H](C)O)NC(=O)[C@H](CCN)NC(=O)[C@H](CCN)NC(=O)[C@H](CC(C)C)NC5=O)cc4)cc3)c3ccc(F)cc32)c1C] |
| 56 | 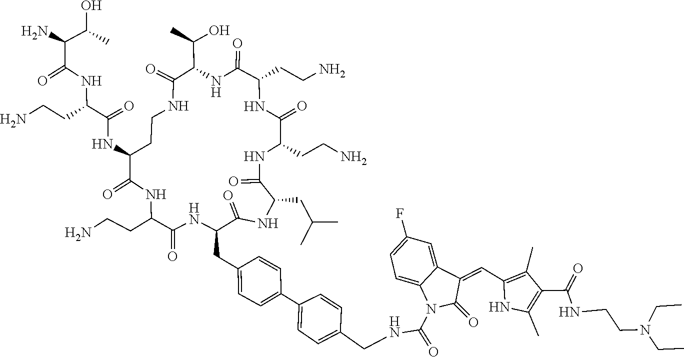 [SMILES: CCN(CC)CCNC(=O)c1c(C)[nH]c(/C=C2\C(=O)N(C(=O)NCc3ccc(-c4ccc(C[C@H]5NC(=O)C(CCN)NC(=O)[C@@H](NC(=O)[C@H](CCN)NC(=O)[C@@H](N)[C@@H](C)O)CCNC(=O)[C@H]([C@@H](C)O)NC(=O)[C@H](CCN)NC(=O)[C@H](CCN)NC(=O)[C@H](CC(C)C)NC5=O)cc4)cc3)c3ccc(F)cc32)c1C] |
| 57 | 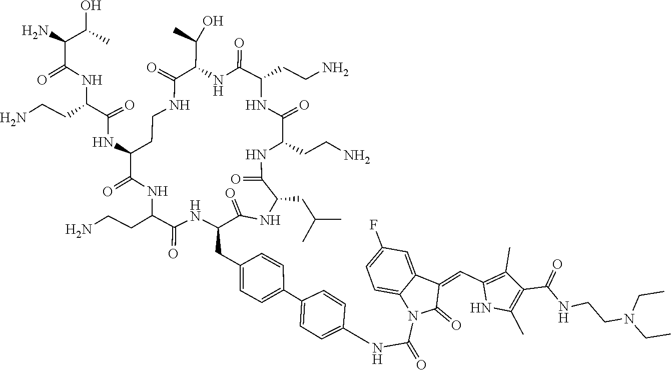 [SMILES: CCN(CC)CCNC(=O)c1c(C)[nH]c(/C=C2\C(=O)N(C(=O)Nc3ccc(-c4ccc(C[C@H]5NC(=O)C(CCN)NC(=O)[C@@H](NC(=O)[C@H](CCN)NC(=O)[C@@H](N)[C@@H](C)O)CCNC(=O)[C@H]([C@@H](C)O)NC(=O)[C@H](CCN)NC(=O)[C@H](CCN)NC(=O)[C@H](CC(C)C)NC5=O)cc4)cc3)c3ccc(F)cc32)c1C] |
| 58 | 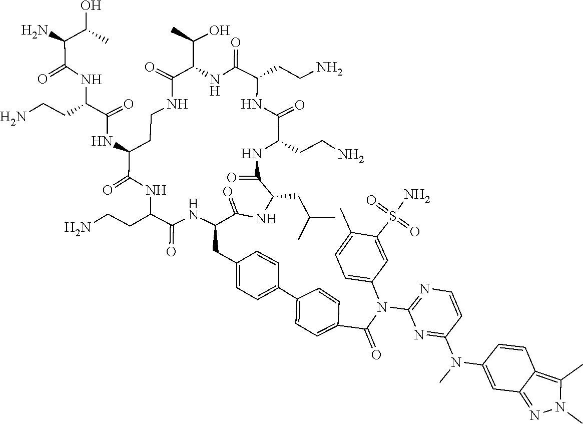 |
| 59 | 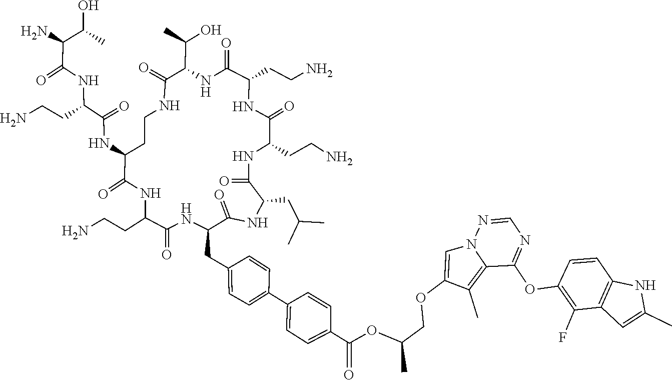 [SMILES: Cc1cc2c(F)c(Oc3ncnn4cc(OC[C@@H](C)OC(=O)c5ccc(-c6ccc(C[C@H]7NC(=O)C(CCN)NC(=O)[C@@H](NC(=O)[C@H](CCN)NC(=O)[C@@H](N)[C@@H](C)O)CCNC(=O)[C@H]([C@@H](C)O)NC(=O)[C@H](CCN)NC(=O)[C@H](CCN)NC(=O)[C@H](CC(C)C)NC7=O)cc6)cc5)c(C)c34)ccc2[nH]1] |
| 60 | 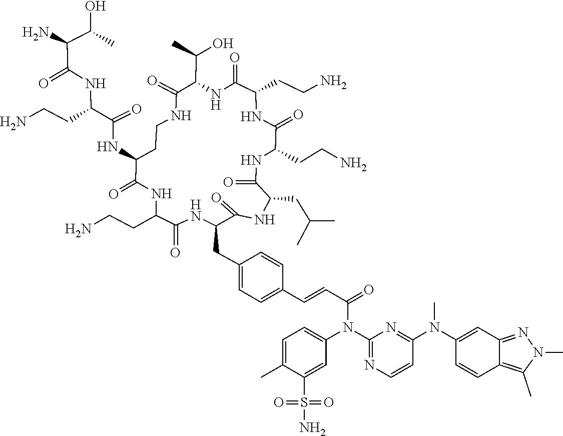 [SMILES: Cc1ccc(N(C(=O)/C=C/c2ccc(C[C@H]3NC(=O)C(CCN)NC(=O)[C@@H](NC(=O)[C@H](CCN)NC(=O)[C@@H](N)[C@@H](C)O)CCNC(=O)[C@H]([C@@H](C)O)NC(=O)[C@H](CCN)NC(=O)[C@H](CCN)NC(=O)[C@H](CC(C)C)NC3=O)cc2)c2nccc(N(C)c3ccc4c(C)n(C)nc4c3)n2)cc1S(N)(=O)=O] |
| 61 | 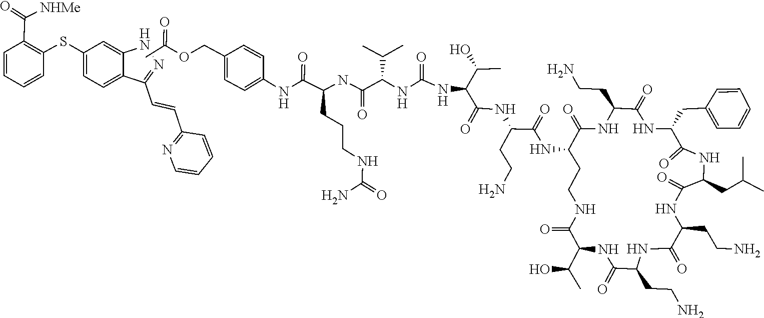 |
| 62 | 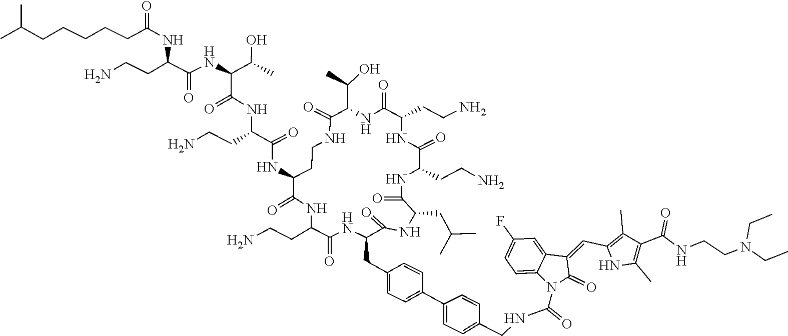 [SMILES: CCN(CC)CCNC(=O)c1c(C)[nH]c(/C=C2\C(=O)N(C(=O)NCc3ccc(-c4ccc(C[C@H]5NC(=O)C(CCN)NC(=O)[C@@H](NC(=O)[C@H](CCN)NC(=O)[C@@H](NC(=O)[C@@H](CCN)NC(=O)CCCCCC(C)C)[C@@H](C)O)CCNC(=O)[C@H]([C@@H](C)O)NC(=O)[C@H](CCN)NC(=O)[C@H](CCN)NC(=O)[C@H](CC(C)C)NC5=O)cc4)cc3)c3ccc(F)cc32)c1C] |
| 63 | 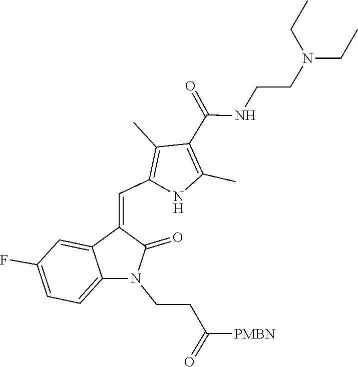 |
| 64 | 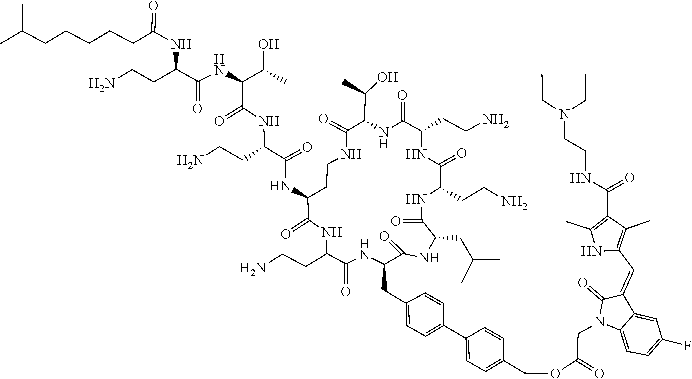 [SMILES: CCN(CC)CCNC(=O)c1c(C)[nH]c(/C=C2\C(=O)N(CC(=O)OCc3ccc(-c4ccc(C[C@H]5NC(=O)C(CCN)NC(=O)[C@@H](NC(=O)[C@H](CCN)NC(=O)[C@@H](NC(=O)[C@@H](CCN)NC(=O)CCCCCC(C)C)[C@@H](C)O)CCNC(=O)[C@H]([C@@H](C)O)NC(=O)[C@H](CCN)NC(=O)[C@H](CCN)NC(=O)[C@H](CC(C)C)NC5=O)cc4)cc3)c3ccc(F)cc32)c1C] |
| 65 | 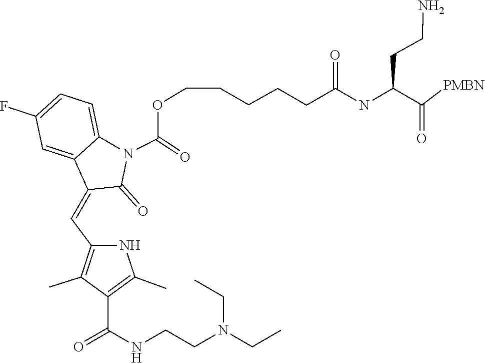 |
| 66 | 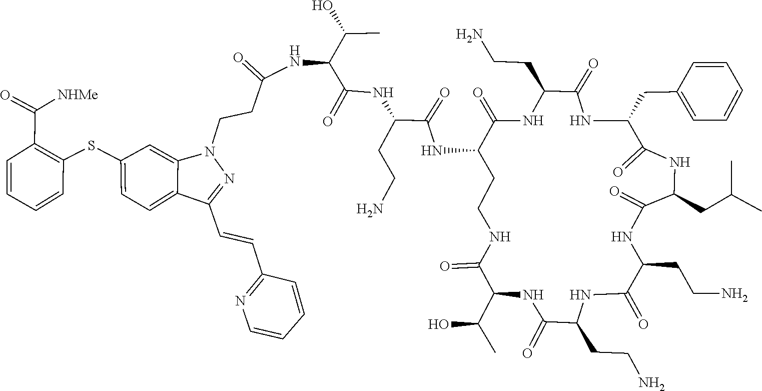 [SMILES: CNC(=O)c1ccccc1Sc1ccc2c(/C=C/c3ccccn3)nn(CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 67 | 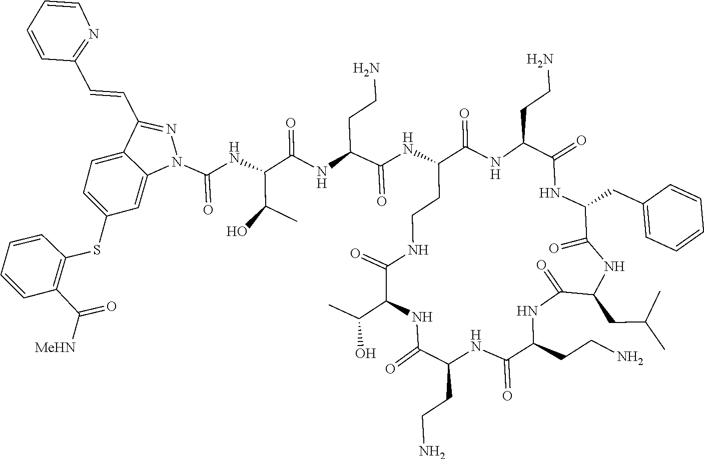 [SMILES: CNC(=O)c1ccccc1Sc1ccc2c(/C=C/c3ccccn3)nn(C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 68 | 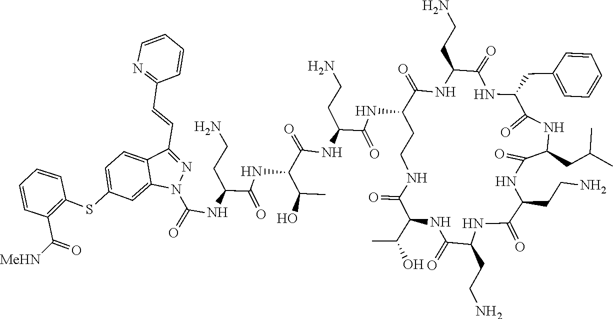 [SMILES: CNC(=O)c1ccccc1Sc1ccc2c(/C=C/c3ccccn3)nn(C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 69 | 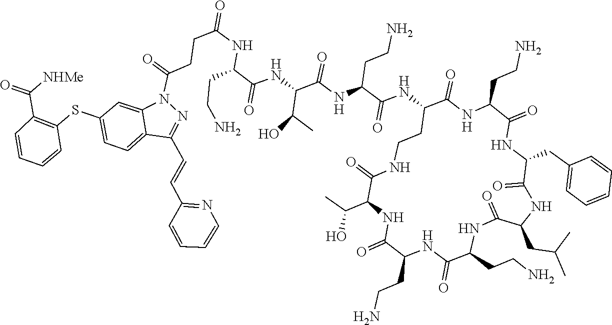 [SMILES: CNC(=O)c1ccccc1Sc1ccc2c(/C=C/c3ccccn3)nn(C(=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 70 | 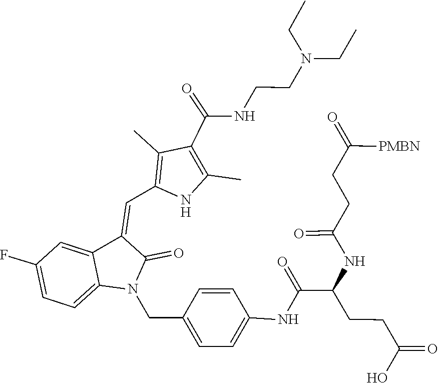 |
| 71 | 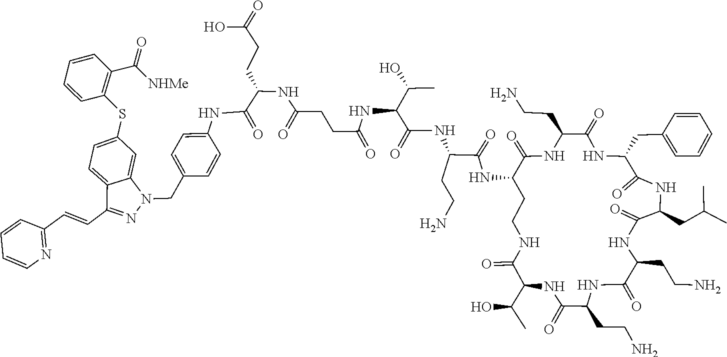 [SMILES: CNC(=O)c1ccccc1Sc1ccc2c(/C=C/c3ccccn3)nn(Cc3ccc(NC(=O)[C@H](CCC(=O)O)NC(=O)CCC(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c2c1] |
| 72 | 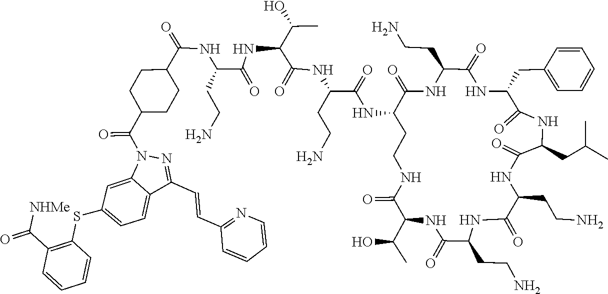 [SMILES: CNC(=O)c1ccccc1Sc1ccc2c(/C=C/c3ccccn3)nn(C(=O)C3CCC(C(=O)N[C@@H](CCN)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)CC3)c2c1] |
| 73 | 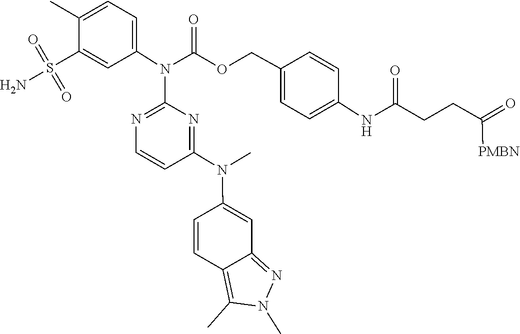 |
| 74 | 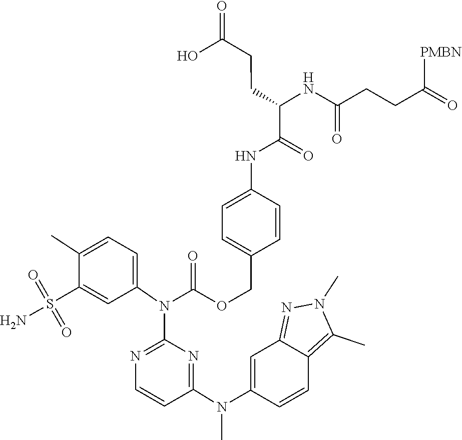 [SMILES: Cc1ccc(N(C(=O)OCc2ccc(NC(=O)[C@H](CCC(=O)O)NC(=O)CCC=O)cc2)c2nccc(N(C)c3ccc4c(C)n(C)nc4c3)n2)cc1S(N)(=O)=O ; "PMBN"] |
| 75 | 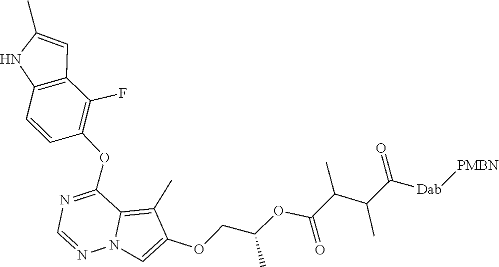 |
| 76 | 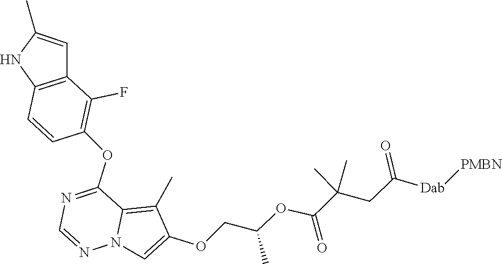 |
| 77 | 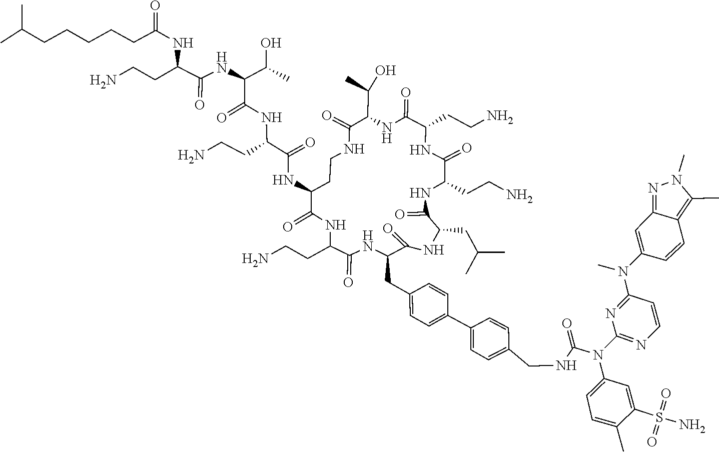 |
| 78 | 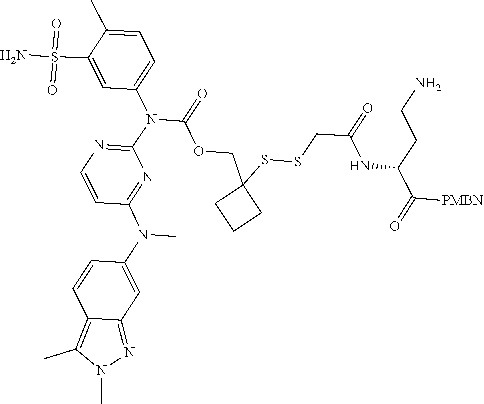 |
| 79 | 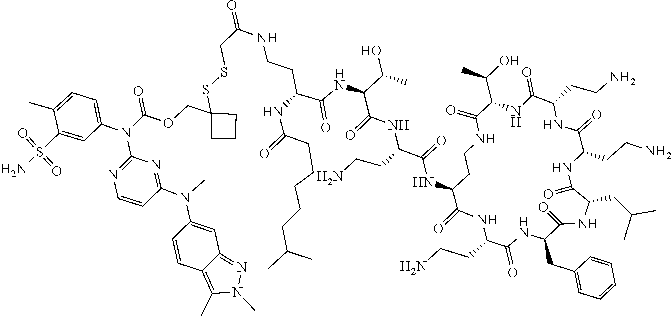 [SMILES: Cc1ccc(N(C(=O)OCC2(SSCC(=O)NCC[C@@H](NC(=O)CCCCCC(C)C)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)CCC2)c2nccc(N(C)c3ccc4c(C)n(C)nc4c3)n2)cc1S(N)(=O)=O] |
| 80 | 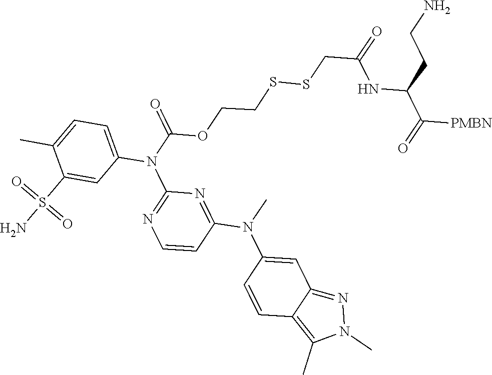 |
| 81 | 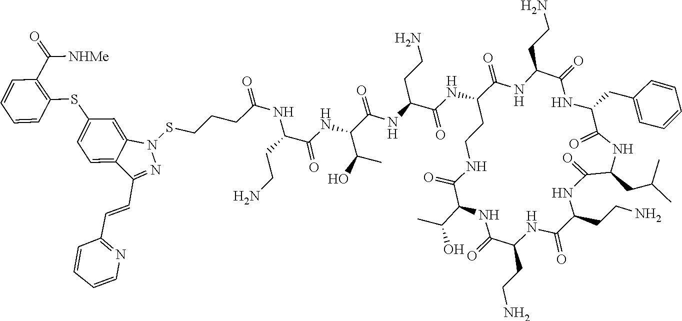 [SMILES: CNC(=O)c1ccccc1Sc1ccc2c(/C=C/c3ccccn3)nn(SCCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 82 | 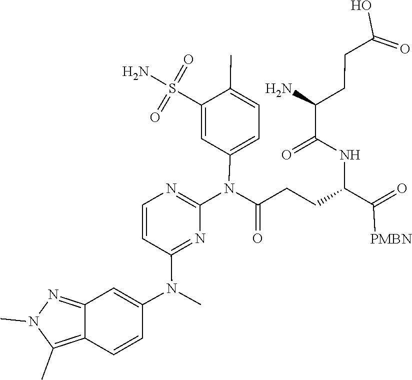 |
| 83 | 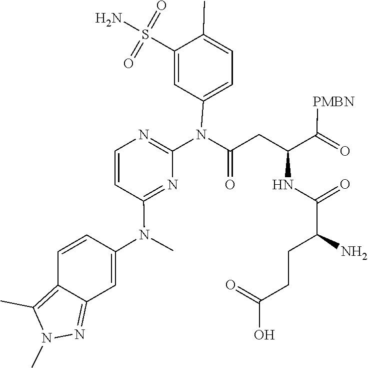 |
| 84 | 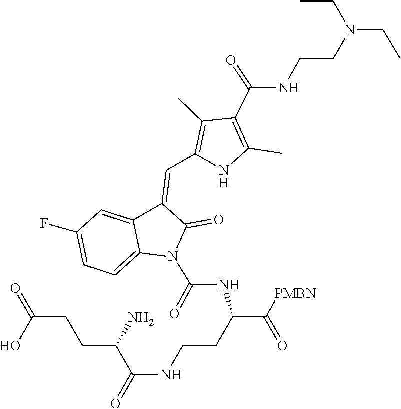 |
| 85 | 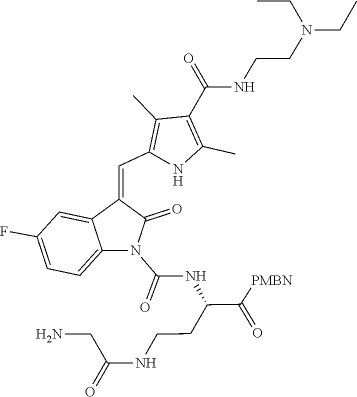 |
| 86 | 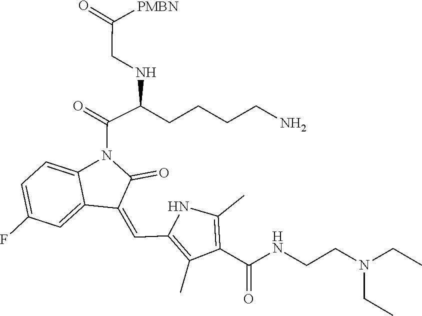 |
| 87 | 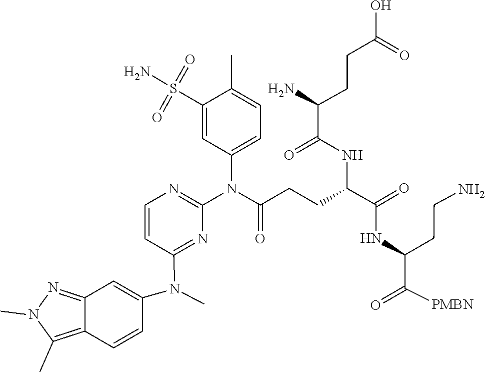 |
| 88 | 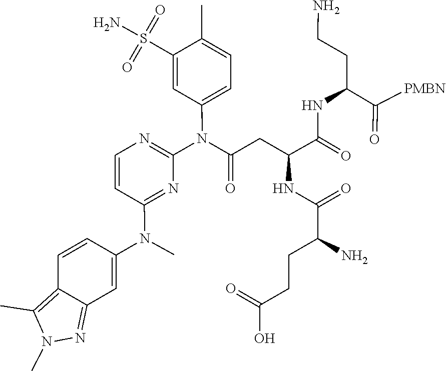 |
| 89 | 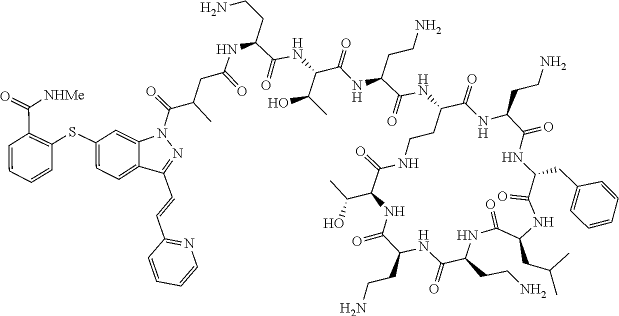 [SMILES: CNC(=O)c1ccccc1Sc1ccc2c(/C=C/c3ccccn3)nn(C(=O)C(C)CC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 90 | 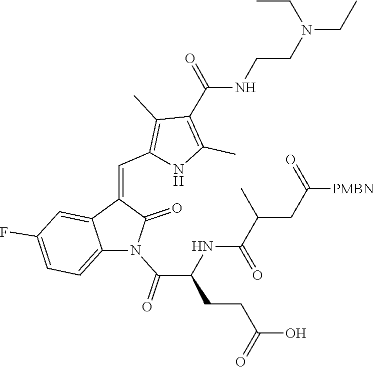 |
| 91 | 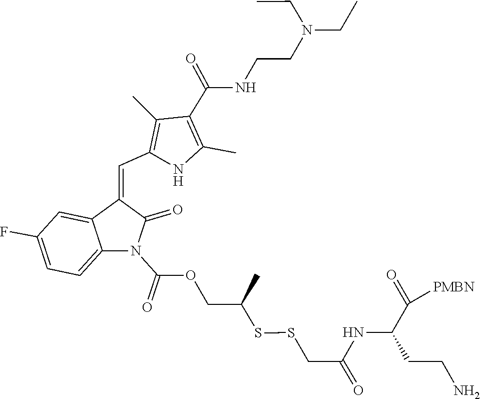 |
| 92 | 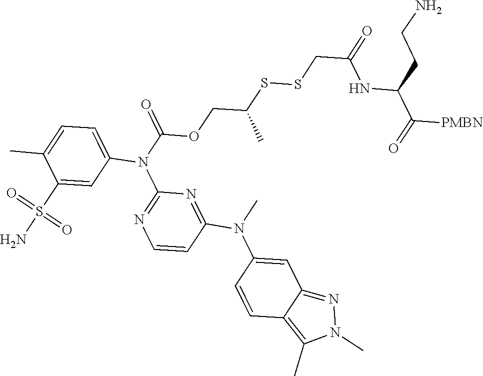 |
| 93 | 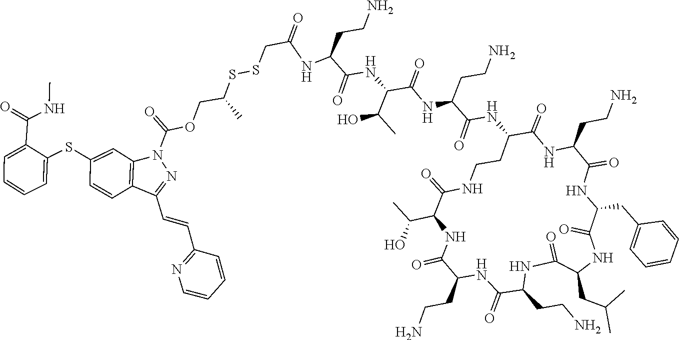 [SMILES: CNC(=O)c1ccccc1Sc1ccc2c(/C=C/c3ccccn3)nn(C(=O)OC[C@@H](C)SSCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 94 | 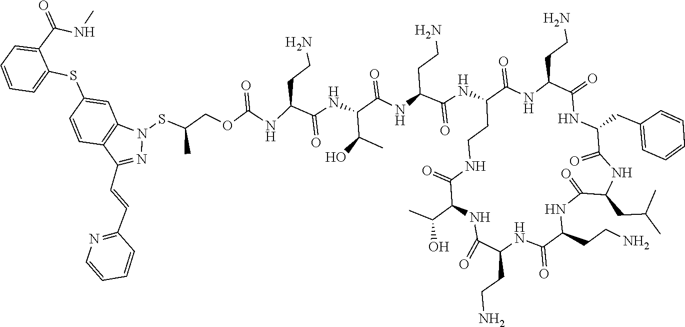 [SMILES: CNC(=O)c1ccccc1Sc1ccc2c(/C=C/c3ccccn3)nn(S[C@H](C)COC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 95 | 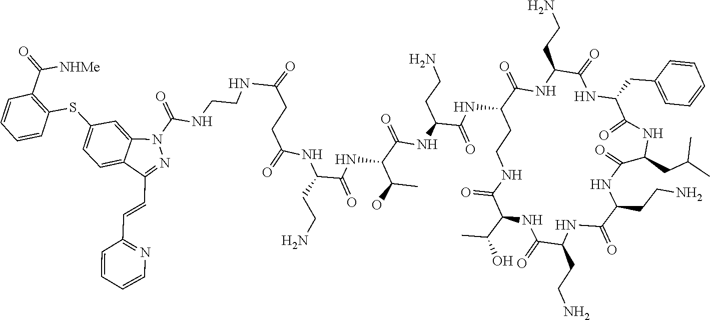 |
| 96 | 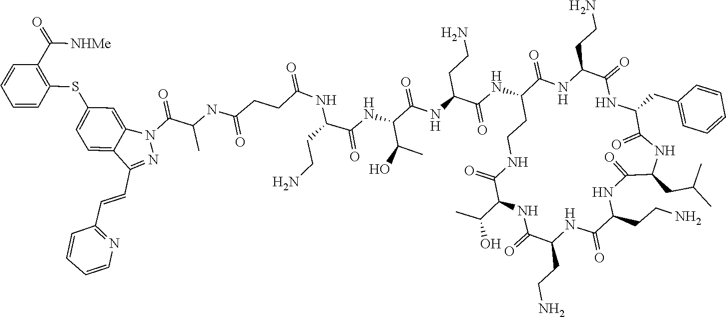 |
| 97 | 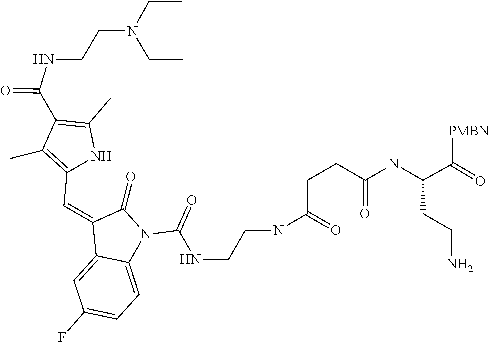 |
| 98 | 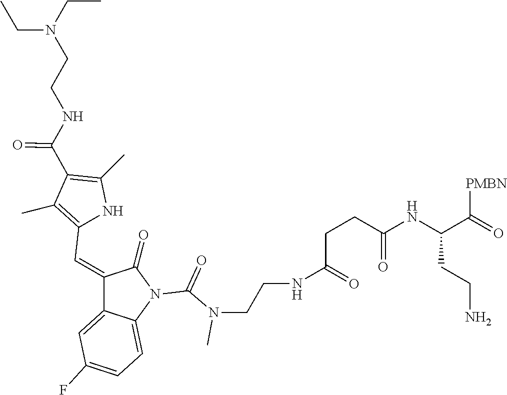 |
| 99 | 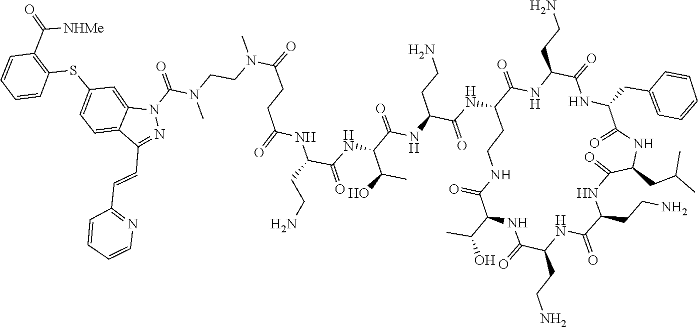 [SMILES: CNC(=O)c1ccccc1Sc1ccc2c(/C=C/c3ccccn3)nn(C(=O)N(C)CCN(C)C(=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 100 | 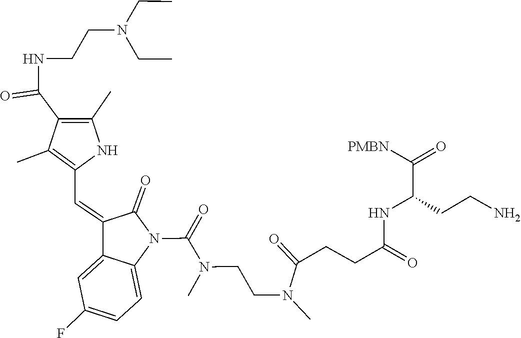 |
| 101 | 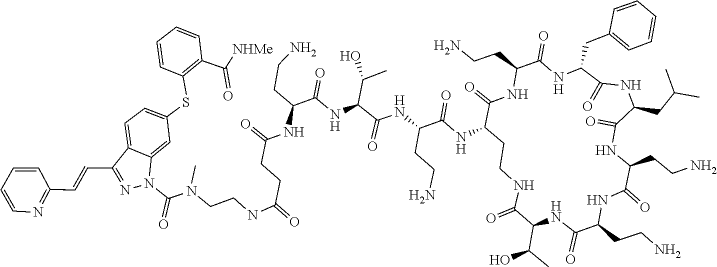 |
| 102 | 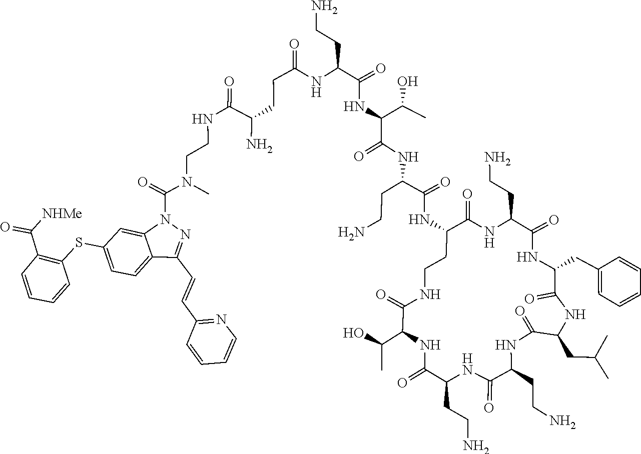 [SMILES: CNC(=O)c1ccccc1Sc1ccc2c(/C=C/c3ccccn3)nn(C(=O)N(C)CCNC(=O)[C@@H](N)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 103 | 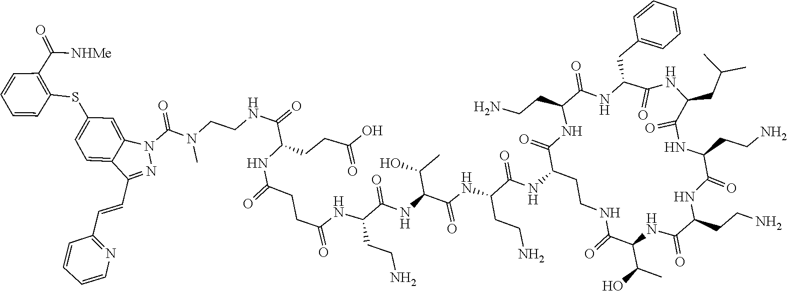 [SMILES: CNC(=O)c1ccccc1Sc1ccc2c(/C=C/c3ccccn3)nn(C(=O)N(C)CCNC(=O)[C@H](CCC(=O)O)NC(=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 104 | 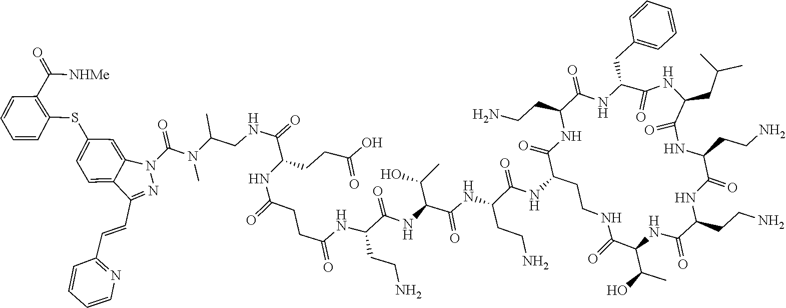 [SMILES: CNC(=O)c1ccccc1Sc1ccc2c(/C=C/c3ccccn3)nn(C(=O)N(C)C(C)CNC(=O)[C@H](CCC(=O)O)NC(=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 105 | 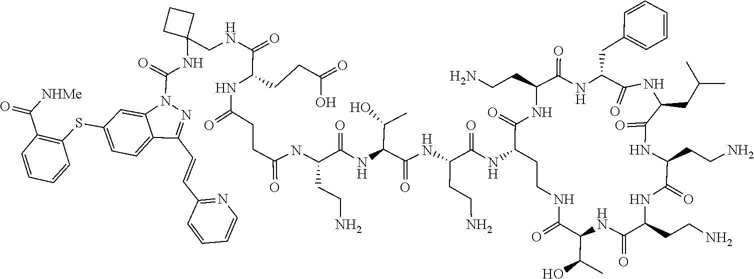 |
| 106 | 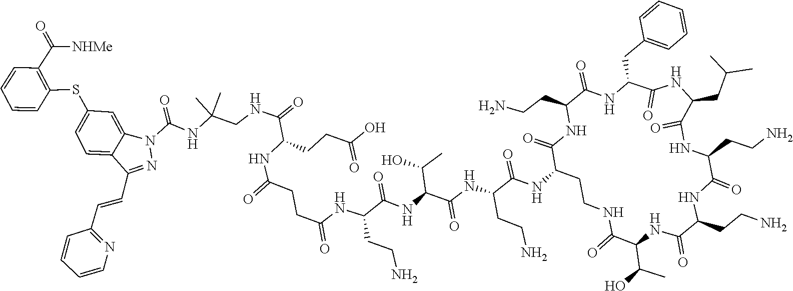 [SMILES: CNC(=O)c1ccccc1Sc1ccc2c(/C=C/c3ccccn3)nn(C(=O)NC(C)(C)CNC(=O)[C@H](CCC(=O)O)NC(=O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 107 | 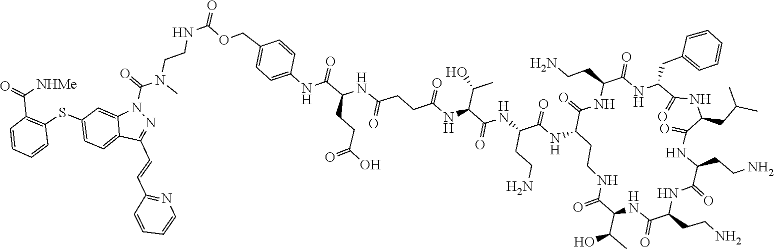 [SMILES: CNC(=O)c1ccccc1Sc1ccc2c(/C=C/c3ccccn3)nn(C(=O)N(C)CCNC(=O)OCc3ccc(NC(=O)[C@H](CCC(=O)O)NC(=O)CCC(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c2c1] |
| 108 | 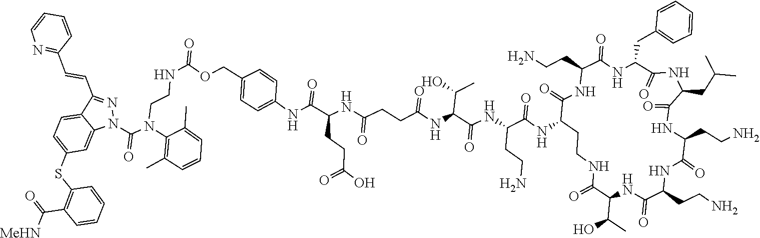 [SMILES: CNC(=O)c1ccccc1Sc1ccc2c(/C=C/c3ccccn3)nn(C(=O)N(CCNC(=O)OCc3ccc(NC(=O)[C@H](CCC(=O)O)NC(=O)CCC(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c3c(C)cccc3C)c2c1] |
| 109 | 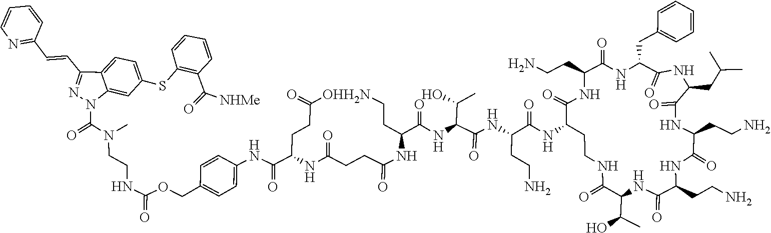 [SMILES: CNC(=O)c1ccccc1Sc1ccc2c(/C=C/c3ccccn3)nn(C(=O)N(C)CCNC(=O)OCc3ccc(NC(=O)[C@H](CCC(=O)O)NC(=O)CCC(=O)N[C@@H](CCN)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c2c1] |
| 110 | 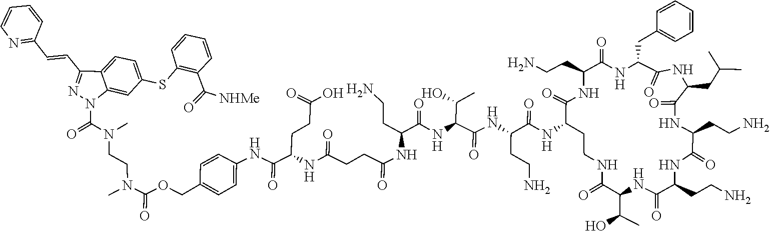 [SMILES: CNC(=O)c1ccccc1Sc1ccc2c(/C=C/c3ccccn3)nn(C(=O)N(C)CCN(C)C(=O)OCc3ccc(NC(=O)[C@H](CCC(=O)O)NC(=O)CCC(=O)N[C@@H](CCN)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cc3)c2c1] |
| 111 | 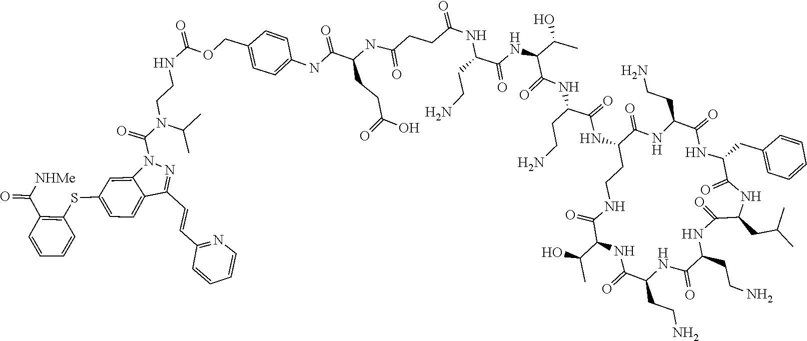 |
| 112 | 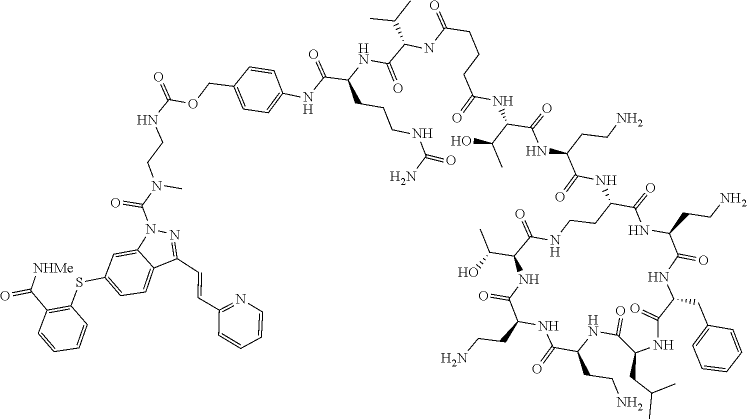 |
| 113 | 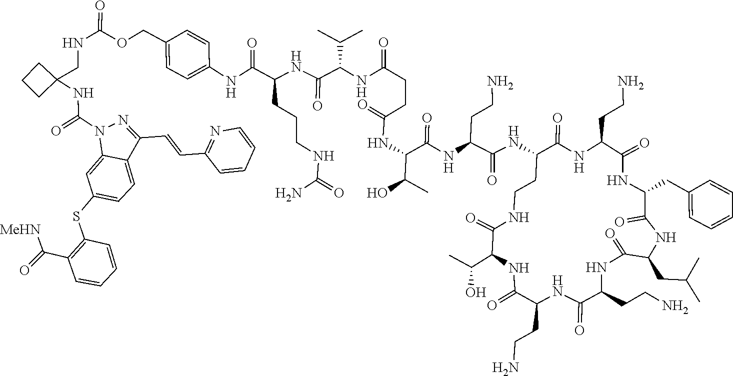 [SMILES: CNC(=O)c1ccccc1Sc1ccc2c(/C=C/c3ccccn3)nn(C(=O)NC3(CNC(=O)OCc4ccc(NC(=O)[C@H](CCCNC(N)=O)NC(=O)[C@@H](NC(=O)CCC(=O)N[C@H](C(=O)N[C@@H](CCN)C(=O)N[C@H]5CCNC(=O)[C@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@@H](C)O)C(C)C)cc4)CCC3)c2c1] |
| 114 | 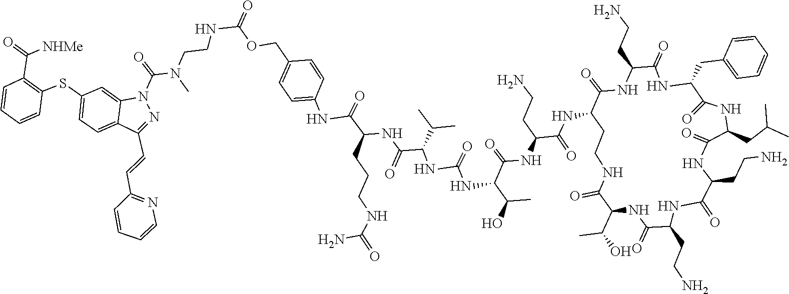 [SMILES: CNC(=O)c1ccccc1Sc1ccc2c(/C=C/c3ccccn3)nn(C(=O)N(C)CCNC(=O)OCc3ccc(NC(=O)[C@H](CCCNC(N)=O)NC(=O)[C@@H](NC(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)C(C)C)cc3)c2c1] |
| 115 | 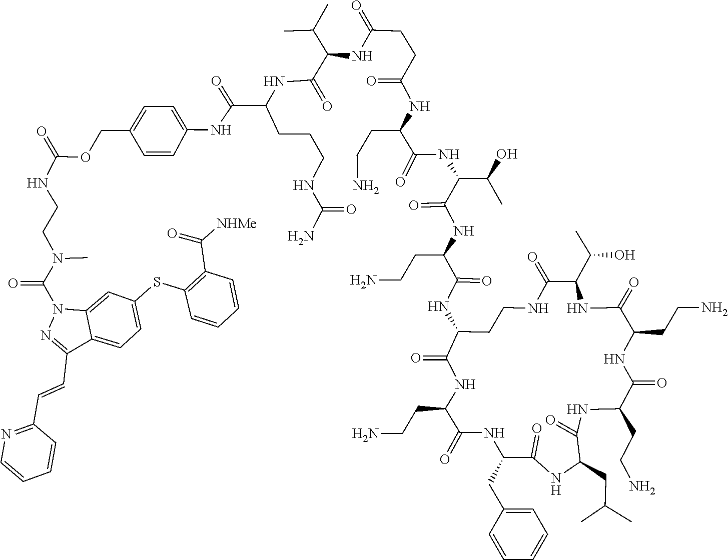 [SMILES: CNC(=O)c1ccccc1Sc1ccc2c(/C=C/c3ccccn3)nn(C(=O)N(C)CCNC(=O)OCc3ccc(NC(=O)C(CCCNC(N)=O)NC(=O)[C@H](NC(=O)CCC(=O)N[C@H](CCN)C(=O)N[C@@H](C(=O)N[C@H](CCN)C(=O)N[C@@H]4CCNC(=O)[C@@H]([C@H](C)O)NC(=O)[C@@H](CCN)NC(=O)[C@@H](CCN)NC(=O)[C@@H](CC(C)C)NC(=O)[C@H](Cc5ccccc5)NC(=O)[C@@H](CCN)NC4=O)[C@H](C)O)C(C)C)cc3)c2c1] |
| 116 | 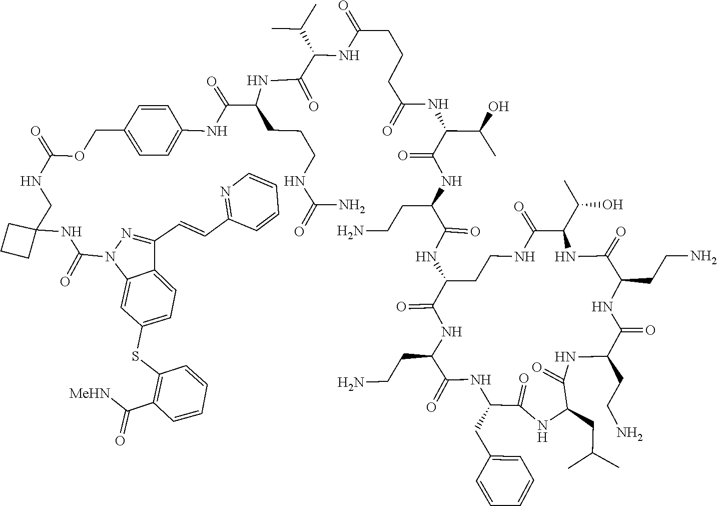 [SMILES: CNC(=O)c1ccccc1Sc1ccc2c(/C=C/c3ccccn3)nn(C(=O)NC3(CNC(=O)OCc4ccc(NC(=O)[C@H](CCCNC(N)=O)NC(=O)[C@@H](NC(=O)CCCC(=O)N[C@@H](C(=O)N[C@H](CCN)C(=O)N[C@@H]5CCNC(=O)[C@@H]([C@H](C)O)NC(=O)[C@@H](CCN)NC(=O)[C@@H](CCN)NC(=O)[C@@H](CC(C)C)NC(=O)[C@H](Cc6ccccc6)NC(=O)[C@@H](CCN)NC5=O)[C@H](C)O)C(C)C)cc4)CCC3)c2c1] |
| 117 | 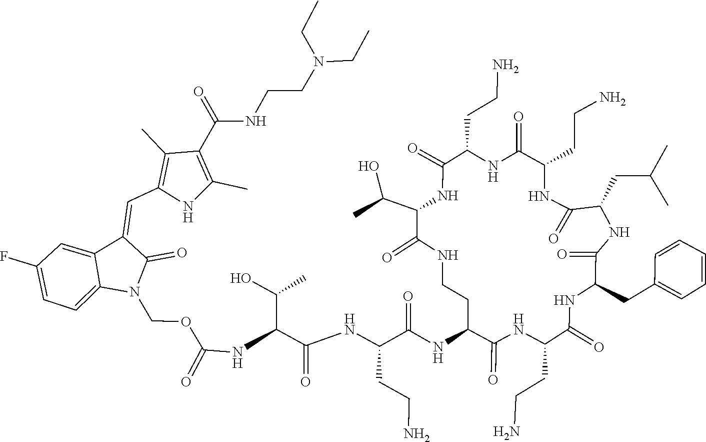 [SMILES: CCN(CC)CCNC(=O)c1c(C)[nH]c(/C=C2\C(=O)N(COC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 118 | 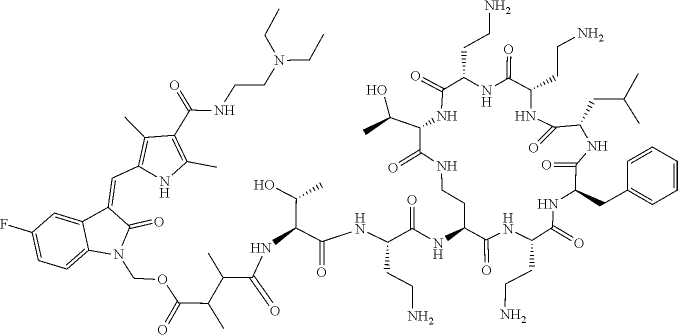 [SMILES: CCN(CC)CCNC(=O)c1c(C)[nH]c(/C=C2\C(=O)N(COC(=O)C(C)C(C)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 119 | 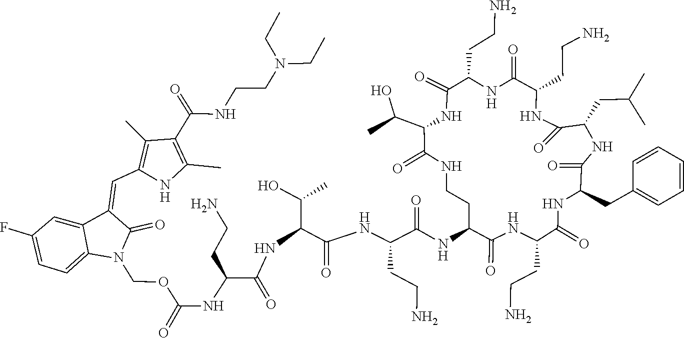 [SMILES: CCN(CC)CCNC(=O)c1c(C)[nH]c(/C=C2\C(=O)N(COC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 120 | 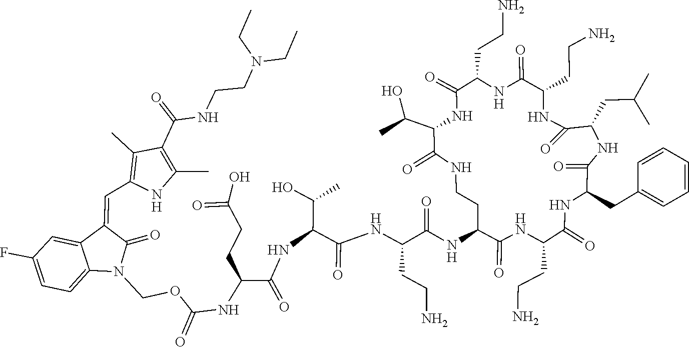 [SMILES: CCN(CC)CCNC(=O)c1c(C)[nH]c(/C=C2\C(=O)N(COC(=O)N[C@@H](CCC(=O)O)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 121 | 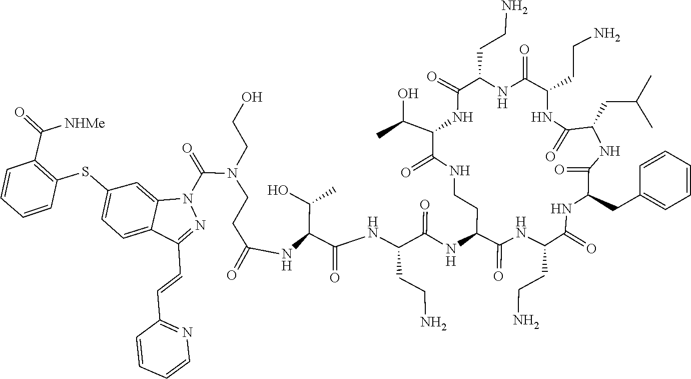 [SMILES: CNC(=O)c1ccccc1Sc1ccc2c(/C=C/c3ccccn3)nn(C(=O)N(CCO)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 122 | 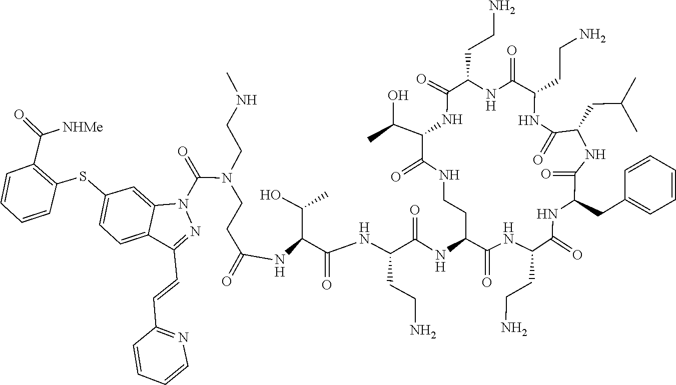 [SMILES: CNCCN(CCC(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O)C(=O)n1nc(/C=C/c2ccccn2)c2ccc(Sc3ccccc3C(=O)NC)cc21] |
| 123 | 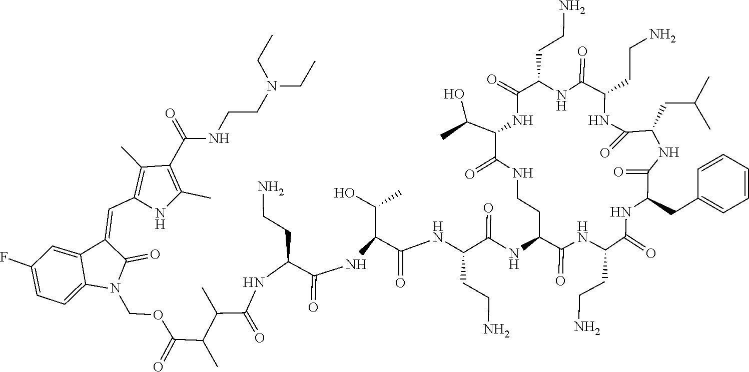 [SMILES: CCN(CC)CCNC(=O)c1c(C)[nH]c(/C=C2\C(=O)N(COC(=O)C(C)C(C)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 124 | 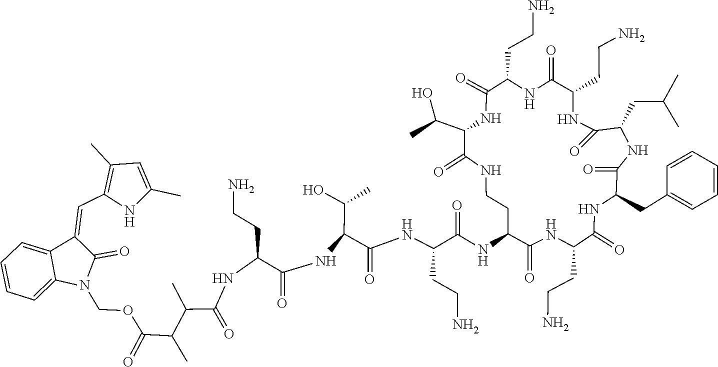 [SMILES: Cc1cc(C)c(/C=C2\C(=O)N(COC(=O)C(C)C(C)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccccc32)[nH]1] |
| 125 | 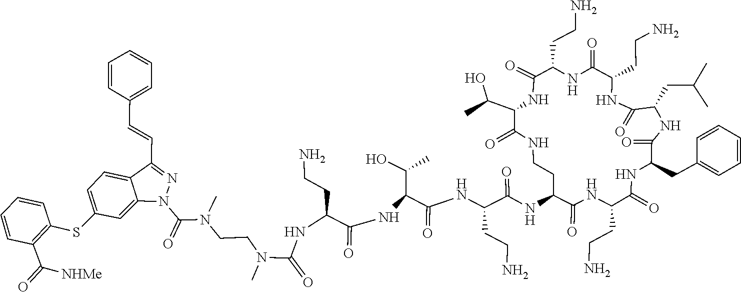 [SMILES: CNC(=O)c1ccccc1Sc1ccc2c(/C=C/c3ccccc3)nn(C(=O)N(C)CCN(C)C(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 126 | 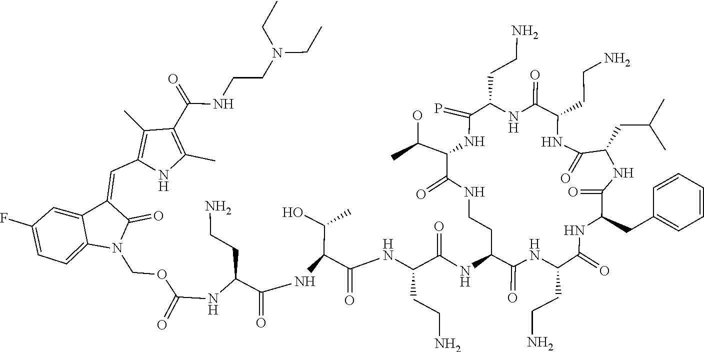 |
| 127 | 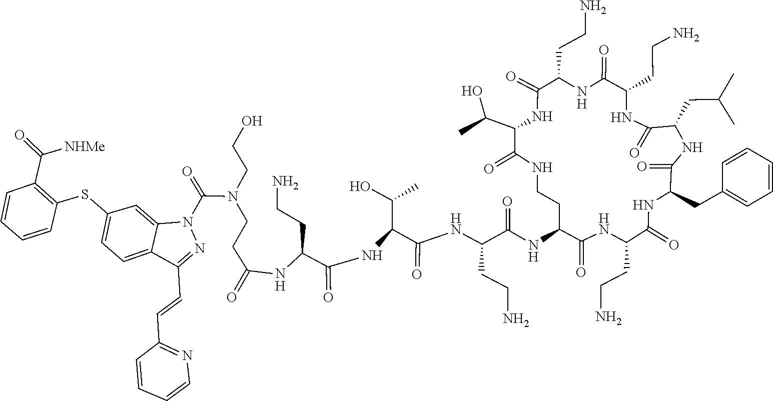 [SMILES: CNC(=O)c1ccccc1Sc1ccc2c(/C=C/c3ccccn3)nn(C(=O)N(CCO)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 128 | 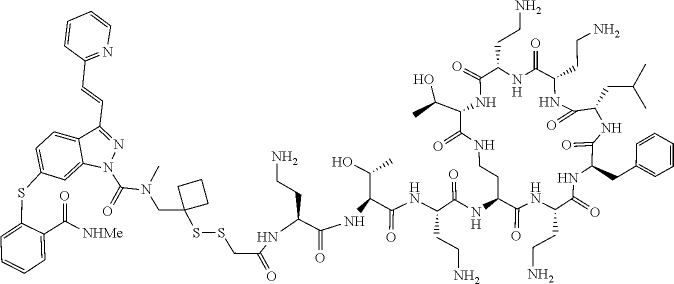 [SMILES: CNC(=O)c1ccccc1Sc1ccc2c(/C=C/c3ccccn3)nn(C(=O)N(C)CC3(SSCC(=O)N[C@@H](CCN)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)CCC3)c2c1] |
| 129 | 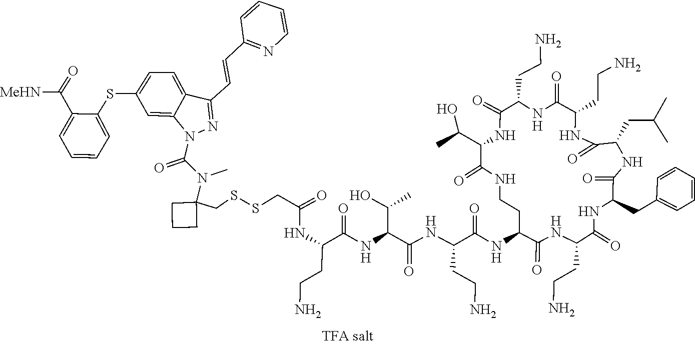 [SMILES: CNC(=O)c1ccccc1Sc1ccc2c(/C=C/c3ccccn3)nn(C(=O)N(C)C3(CSSCC(=O)N[C@@H](CCN)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)CCC3)c2c1] |
| 130 | 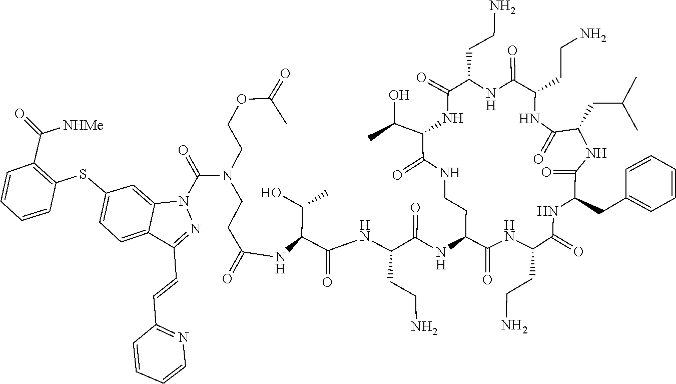 [SMILES: CNC(=O)c1ccccc1Sc1ccc2c(/C=C/c3ccccn3)nn(C(=O)N(CCOC(C)=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 131 | 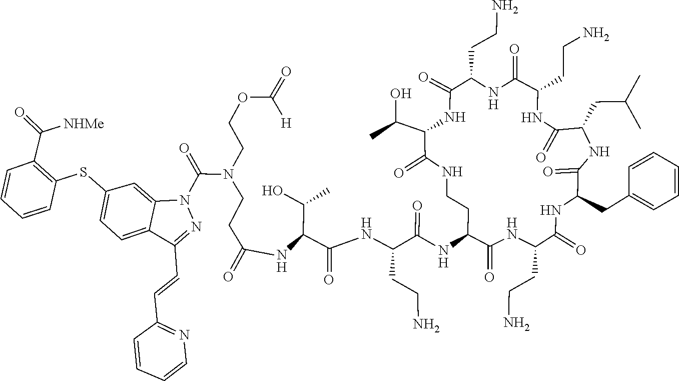 [SMILES: CNC(=O)c1ccccc1Sc1ccc2c(/C=C/c3ccccn3)nn(C(=O)N(CCOC=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 132 | 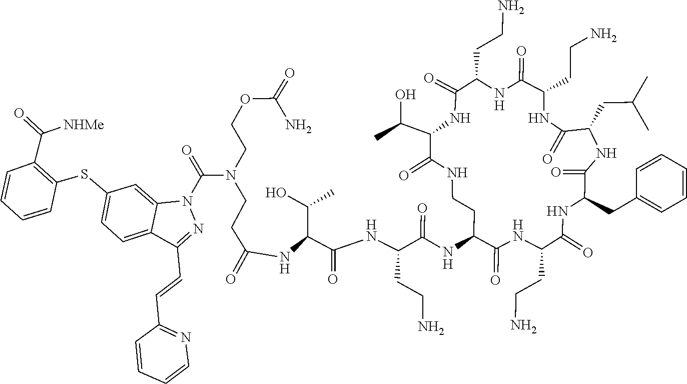 [SMILES: CNC(=O)c1ccccc1Sc1ccc2c(/C=C/c3ccccn3)nn(C(=O)N(CCOC(N)=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 133 | 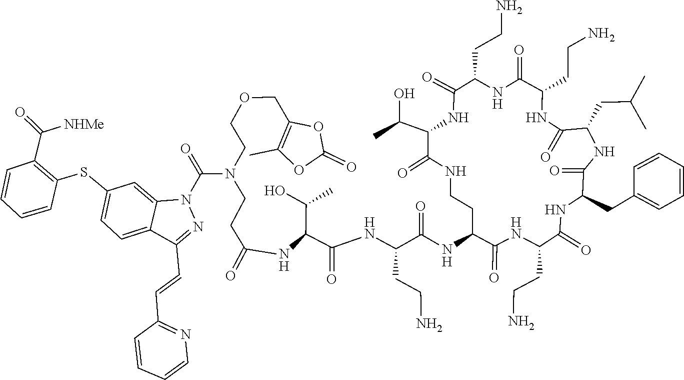 [SMILES: CNC(=O)c1ccccc1Sc1ccc2c(/C=C/c3ccccn3)nn(C(=O)N(CCOCc3oc(=O)oc3C)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 132 | 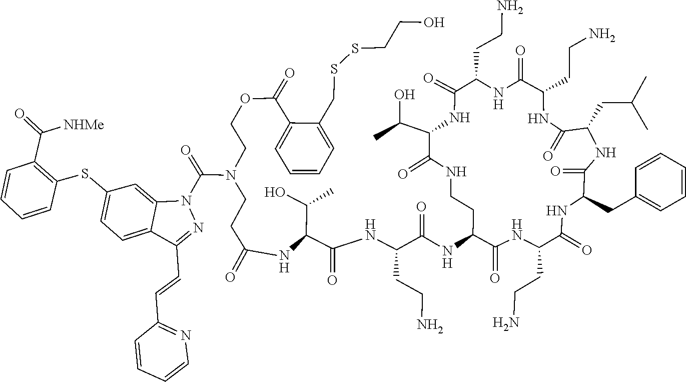 [SMILES: CNC(=O)c1ccccc1Sc1ccc2c(/C=C/c3ccccn3)nn(C(=O)N(CCOC(=O)c3ccccc3CSSCCO)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 133 | 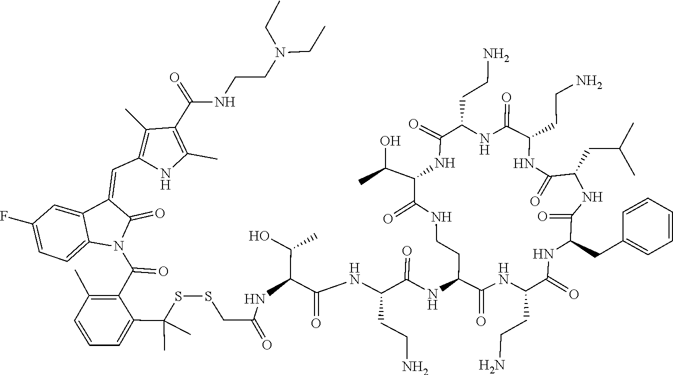 [SMILES: CCN(CC)CCNC(=O)c1c(C)[nH]c(/C=C2\C(=O)N(C(=O)c3c(C)cccc3C(C)(C)SSCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 134 | 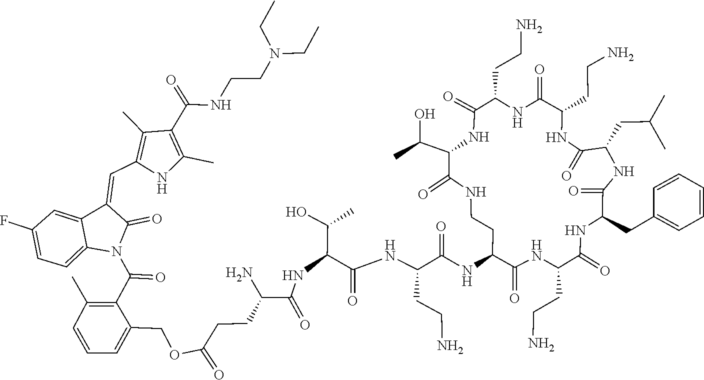 [SMILES: CCN(CC)CCNC(=O)c1c(C)[nH]c(/C=C2\C(=O)N(C(=O)c3c(C)cccc3COC(=O)CC[C@H](N)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 135 | 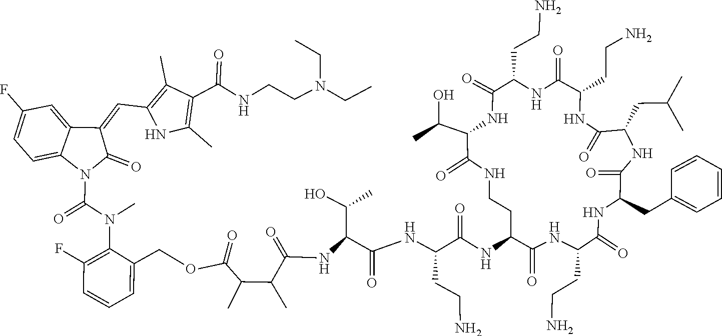 [SMILES: CCN(CC)CCNC(=O)c1c(C)[nH]c(/C=C2\C(=O)N(C(=O)N(C)c3c(F)cccc3COC(=O)C(C)C(C)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 136 | 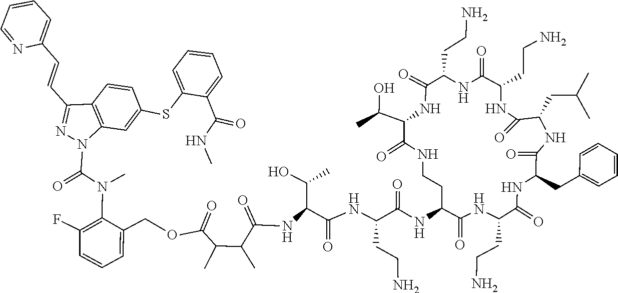 [SMILES: CNC(=O)c1ccccc1Sc1ccc2c(/C=C/c3ccccn3)nn(C(=O)N(C)c3c(F)cccc3COC(=O)C(C)C(C)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 137 | 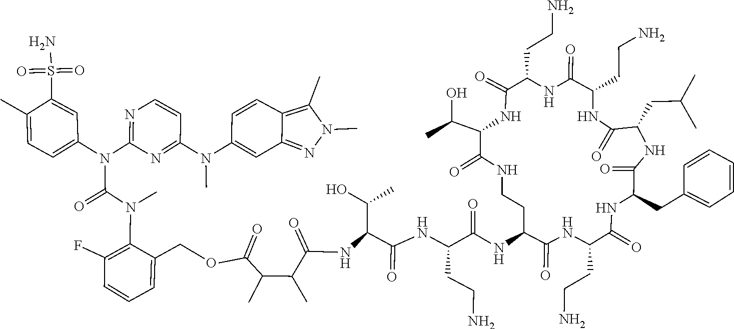 [SMILES: Cc1ccc(N(C(=O)N(C)c2c(F)cccc2COC(=O)C(C)C(C)C(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)c2nccc(N(C)c3ccc4c(C)n(C)nc4c3)n2)cc1S(N)(=O)=O] |
| 138 | 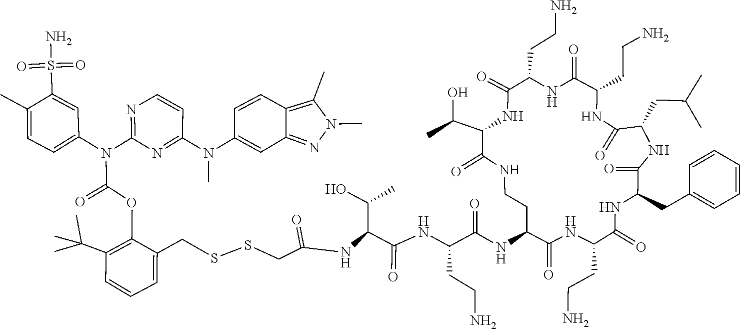 [SMILES: Cc1ccc(N(C(=O)Oc2c(CSSCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)cccc2C(C)(C)C)c2nccc(N(C)c3ccc4c(C)n(C)nc4c3)n2)cc1S(N)(=O)=O] |
| 139 | 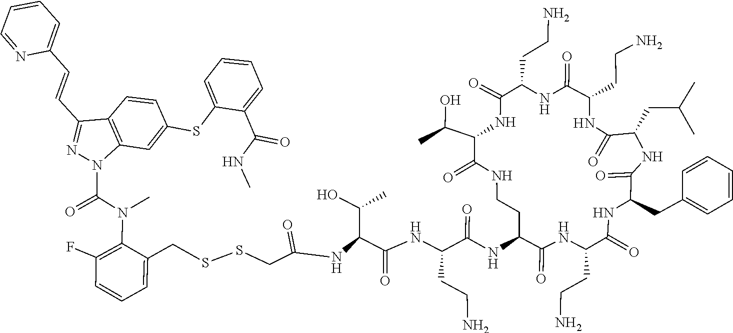 [SMILES: CNC(=O)c1ccccc1Sc1ccc2c(/C=C/c3ccccn3)nn(C(=O)N(C)c3c(F)cccc3CSSCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 140 | 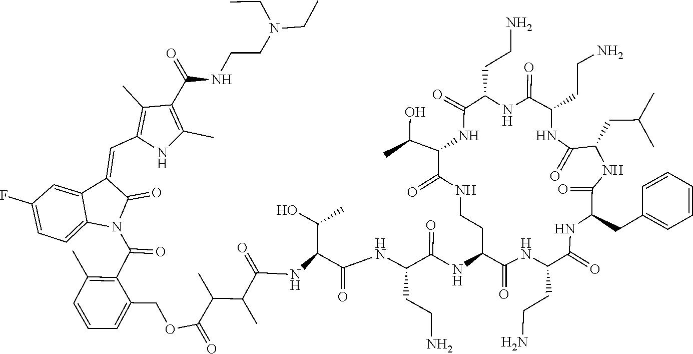 |
| 141 | 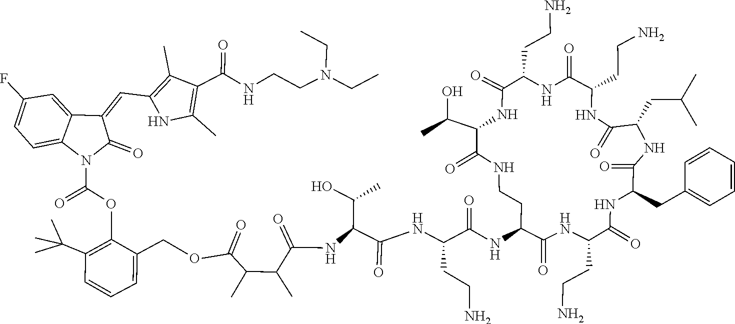 [SMILES: CCN(CC)CCNC(=O)c1c(C)[nH]c(/C=C2\C(=O)N(C(=O)Oc3c(COC(=O)C(C)C(C)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cccc3C(C)(C)C)c3ccc(F)cc32)c1C] |
| 142 | 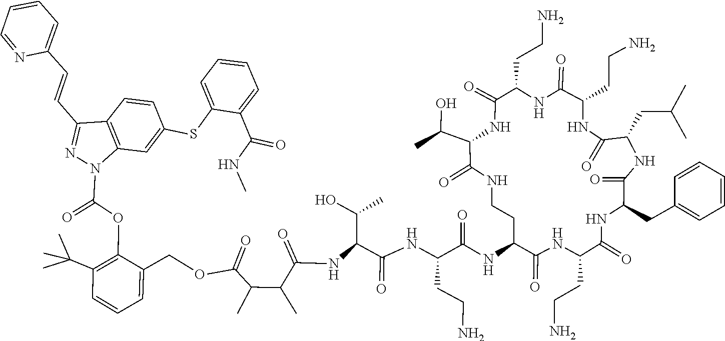 [SMILES: CNC(=O)c1ccccc1Sc1ccc2c(/C=C/c3ccccn3)nn(C(=O)Oc3c(COC(=O)C(C)C(C)C(=O)N[C@H](C(=O)N[C@@H](CCN)C(=O)N[C@H]4CCNC(=O)[C@H]([C@@H](C)O)NC(=O)[C@H](CCN)NC(=O)[C@H](CCN)NC(=O)[C@H](CC(C)C)NC(=O)[C@@H](Cc5ccccc5)NC(=O)[C@H](CCN)NC4=O)[C@@H](C)O)cccc3C(C)(C)C)c2c1] |
| 143 | 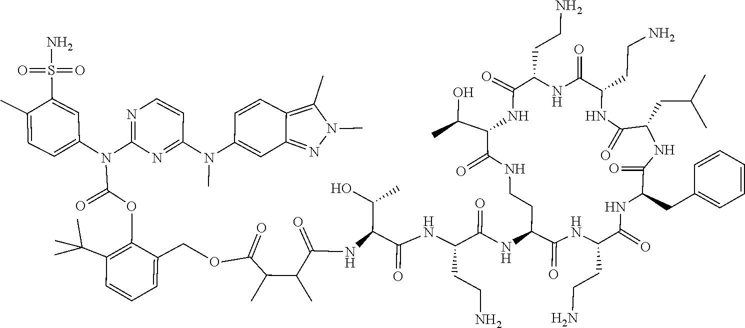 [SMILES: Cc1ccc(N(C(=O)Oc2c(COC(=O)C(C)C(C)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)cccc2C(C)(C)C)c2nccc(N(C)c3ccc4c(C)n(C)nc4c3)n2)cc1S(N)(=O)=O] |
| 144 | 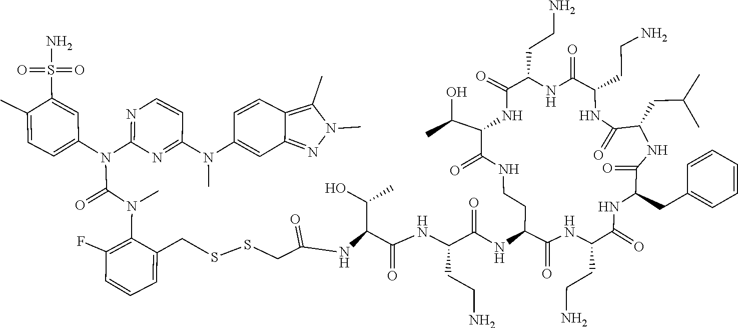 [SMILES: Cc1ccc(N(C(=O)N(C)c2c(F)cccc2CSSCC(=O)N[C@H](C(=O)N[C@@H](CCN)C(=O)N[C@H]2CCNC(=O)[C@H]([C@@H](C)O)NC(=O)[C@H](CCN)NC(=O)[C@H](CCN)NC(=O)[C@H](CC(C)C)NC(=O)[C@@H](Cc3ccccc3)NC(=O)[C@H](CCN)NC2=O)[C@@H](C)O)c2nccc(N(C)c3ccc4c(C)n(C)nc4c3)n2)cc1S(N)(=O)=O] |
| 145 | 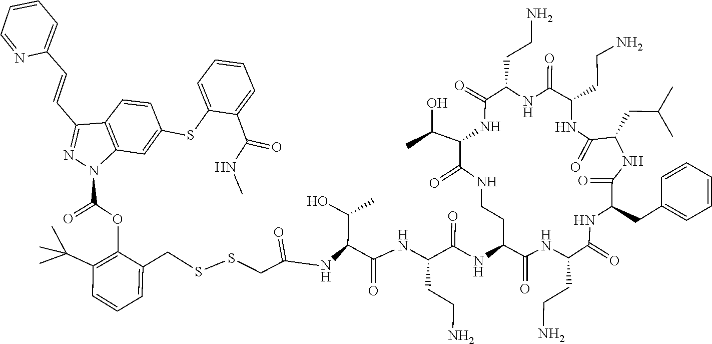 |
| 146 | 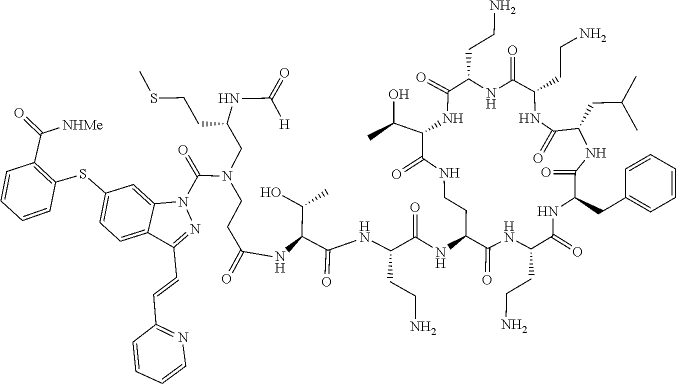 |
| 147 | 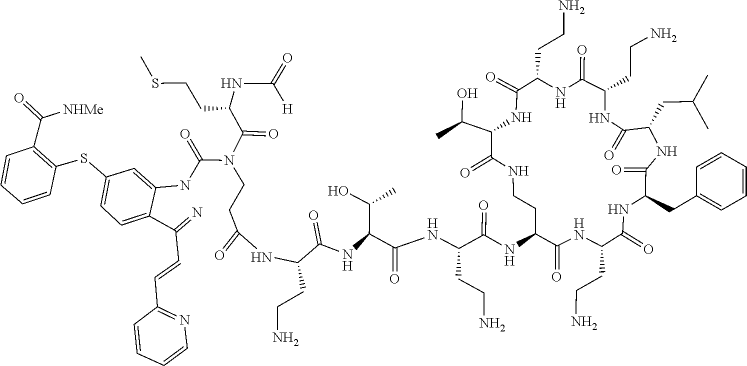 |
| 148 | 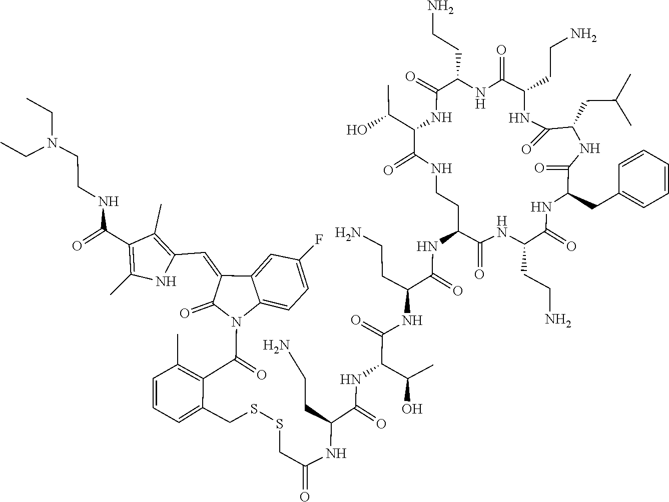 |
| 149 | 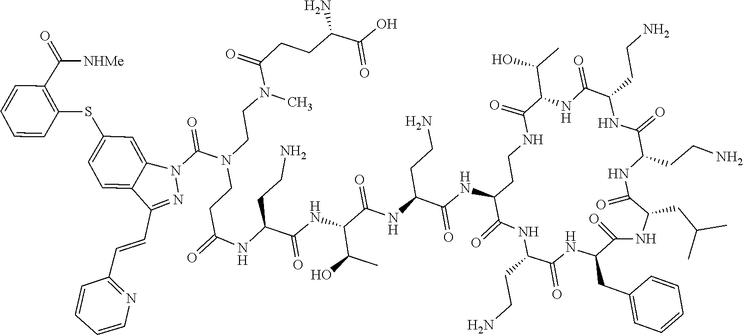 [SMILES: CNC(=O)c1ccccc1Sc1ccc2c(/C=C/c3ccccn3)nn(C(=O)N(CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)CCN(C)C(=O)CC[C@H](N)C(=O)O)c2c1] |
| 150 | 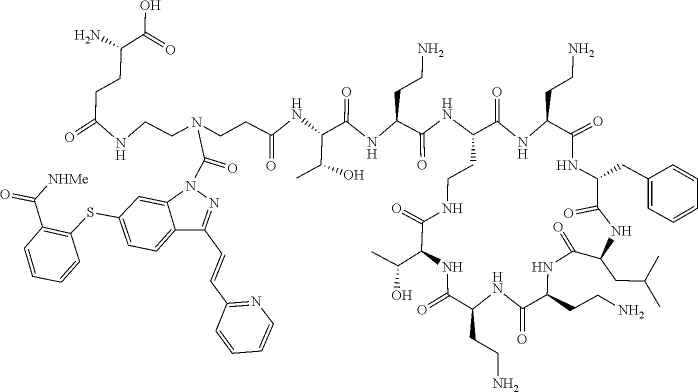 [SMILES: CNC(=O)c1ccccc1Sc1ccc2c(/C=C/c3ccccn3)nn(C(=O)N(CCNC(=O)CC[C@H](N)C(=O)O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 151 | 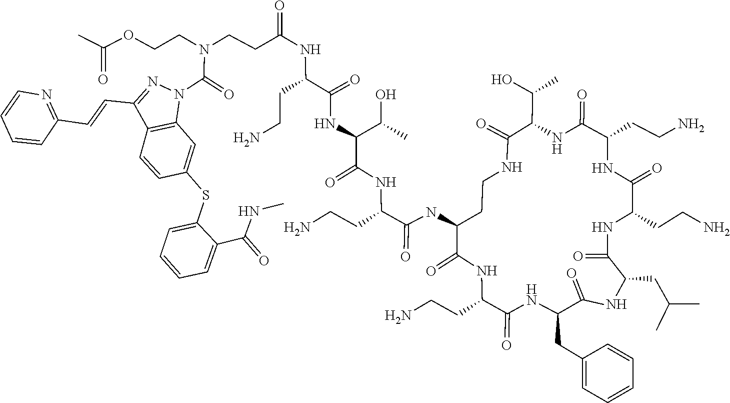 |
| 152 | 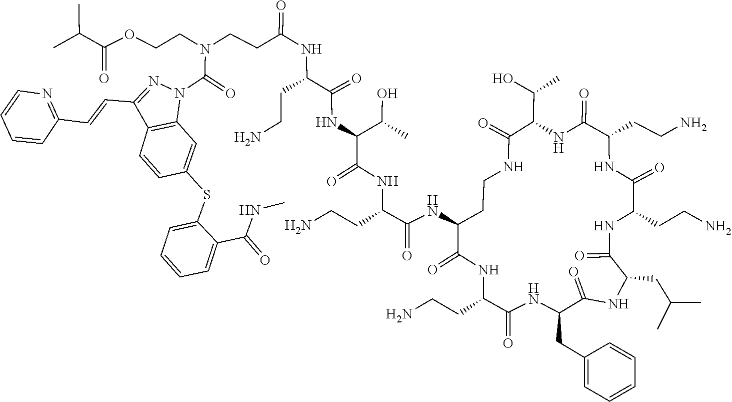 [SMILES: CNC(=O)c1ccccc1Sc1ccc2c(/C=C/c3ccccn3)nn(C(=O)N(CCOC(=O)C(C)C)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 153 | 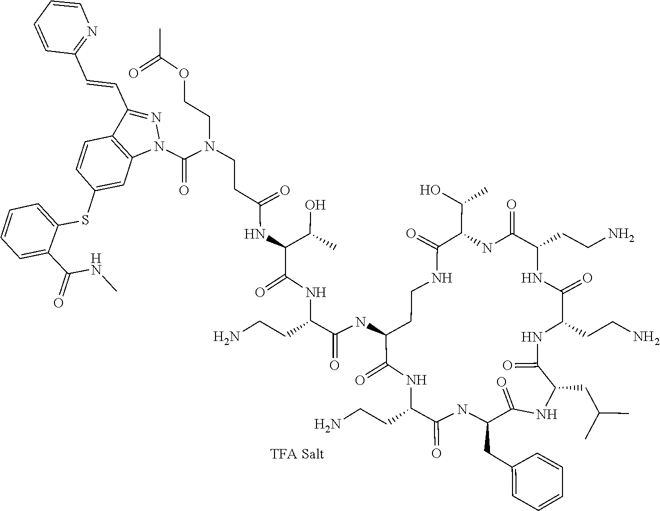 |
| 154 | 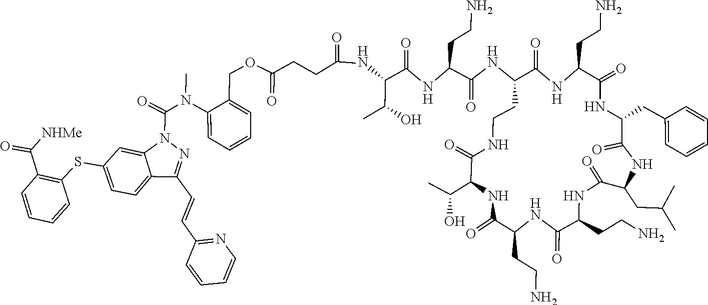 |
| 155 | 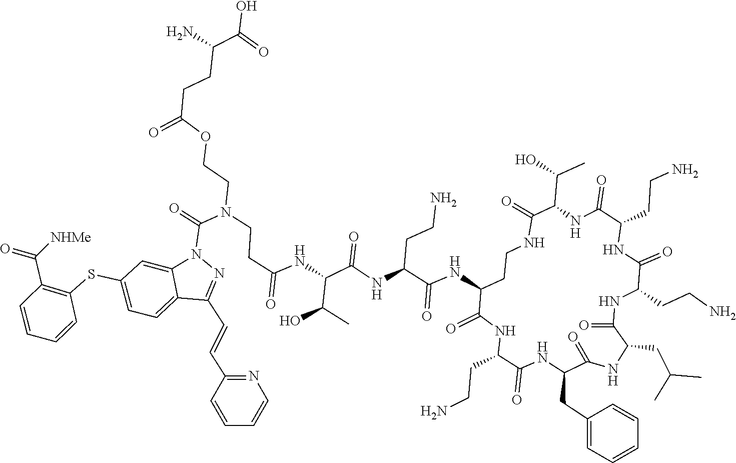 [SMILES: CNC(=O)c1ccccc1Sc1ccc2c(/C=C/c3ccccn3)nn(C(=O)N(CCOC(=O)CC[C@H](N)C(=O)O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 156 | 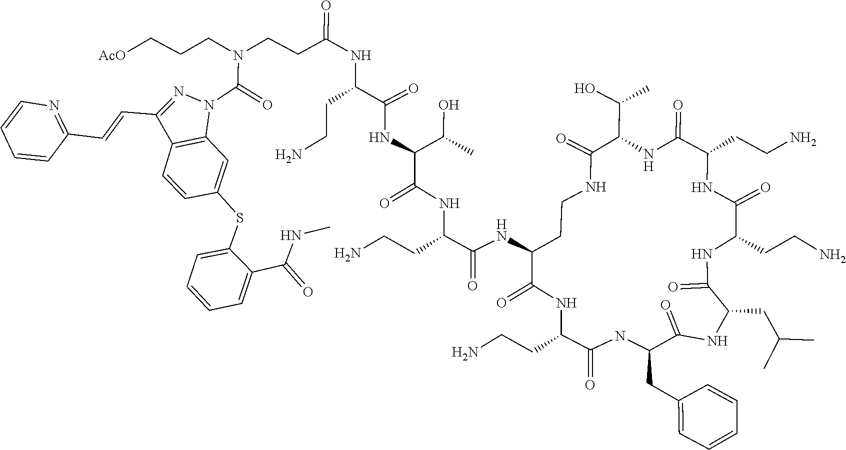 |
| 157 | 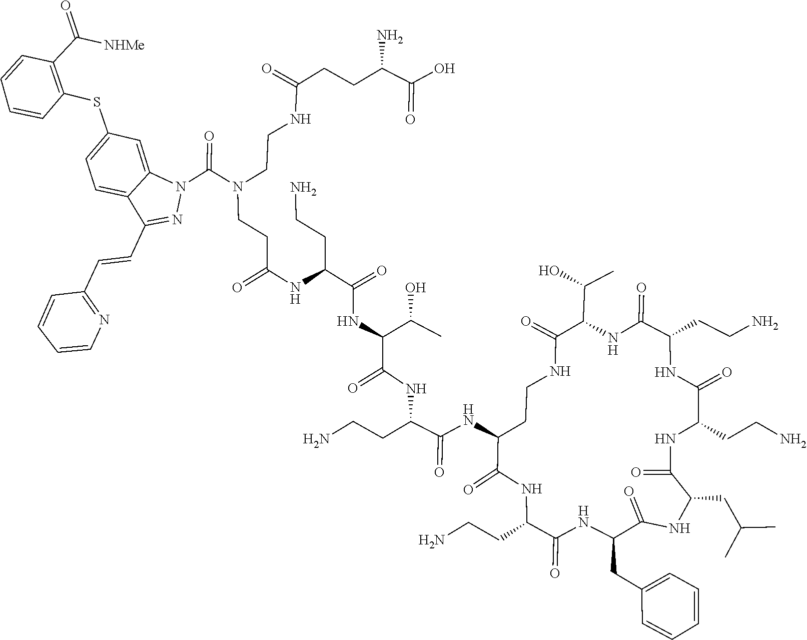 [SMILES: CNC(=O)c1ccccc1Sc1ccc2c(/C=C/c3ccccn3)nn(C(=O)N(CCNC(=O)CC[C@H](N)C(=O)O)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 158 | 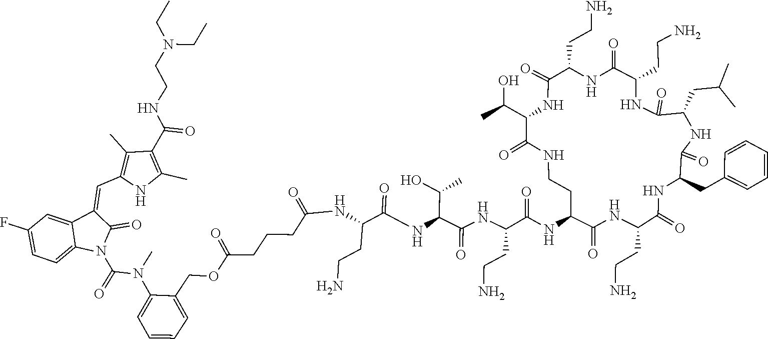 [SMILES: CCN(CC)CCNC(=O)c1c(C)[nH]c(/C=C2\C(=O)N(C(=O)N(C)c3ccccc3COC(=O)CCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 159 | 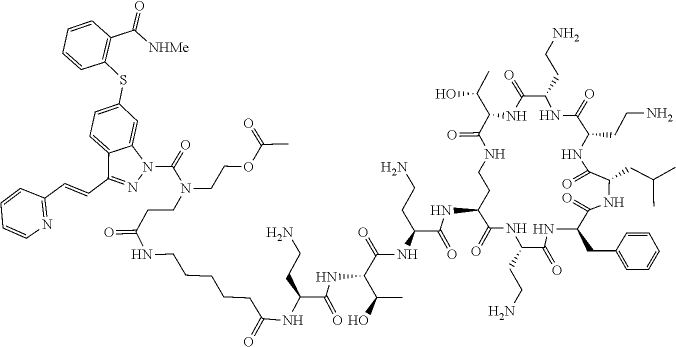 [SMILES: CNC(=O)c1ccccc1Sc1ccc2c(/C=C/c3ccccn3)nn(C(=O)N(CCOC(C)=O)CCC(=O)NCCCCCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 160 | 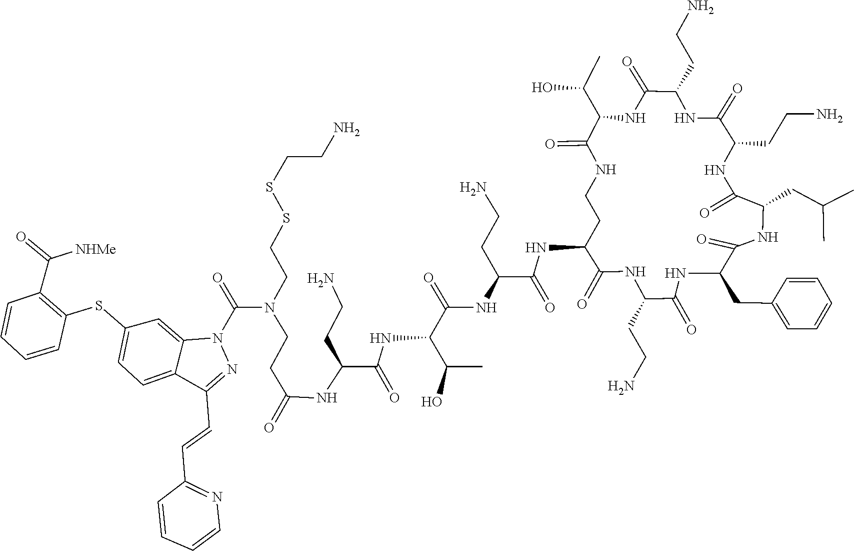 [SMILES: CNC(=O)c1ccccc1Sc1ccc2c(/C=C/c3ccccn3)nn(C(=O)N(CCSSCCN)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 161 | 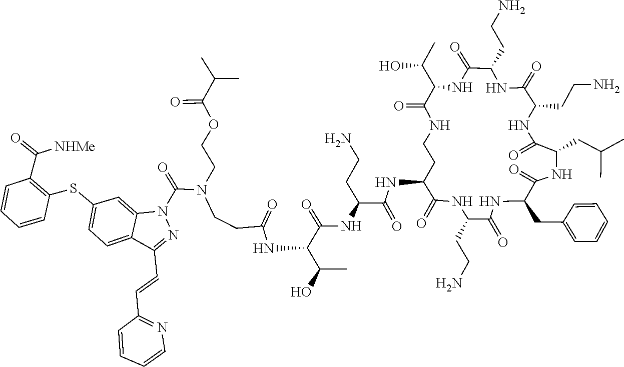 [SMILES: CNC(=O)c1ccccc1Sc1ccc2c(/C=C/c3ccccn3)nn(C(=O)N(CCOC(=O)C(C)C)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 162 | 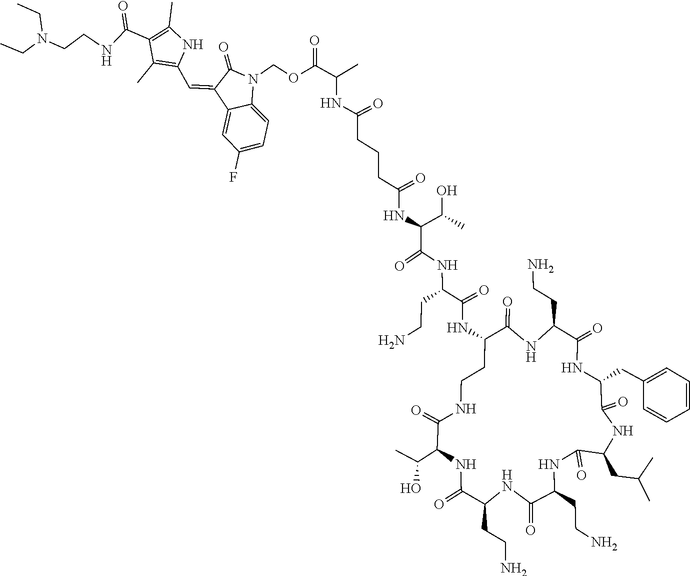 [SMILES: CCN(CC)CCNC(=O)c1c(C)[nH]c(/C=C2\C(=O)N(COC(=O)C(C)NC(=O)CCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c3ccc(F)cc32)c1C] |
| 163 | 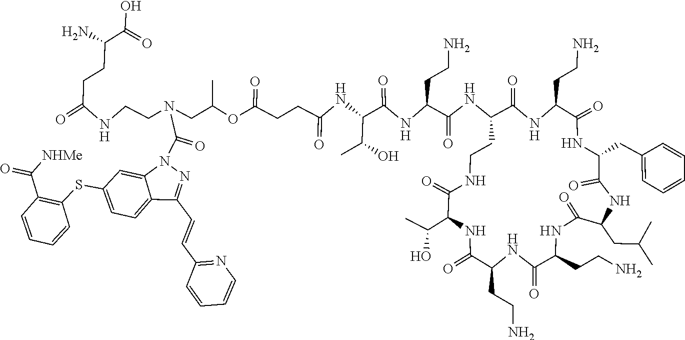 [SMILES: CNC(=O)c1ccccc1Sc1ccc2c(/C=C/c3ccccn3)nn(C(=O)N(CCNC(=O)CC[C@H](N)C(=O)O)CC(C)OC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 164 | 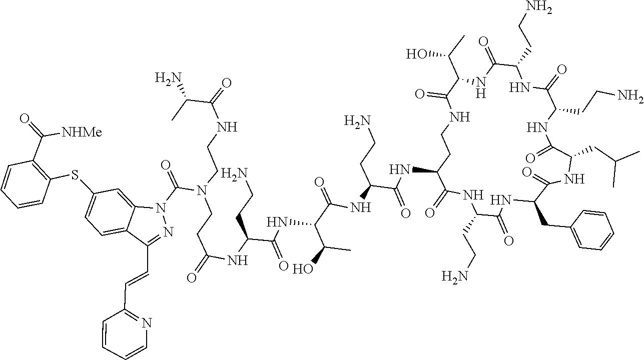 [SMILES: CNC(=O)c1ccccc1Sc1ccc2c(/C=C/c3ccccn3)nn(C(=O)N(CCNC(=O)[C@H](C)N)CCC(=O)N[C@@H](CCN)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 165 | 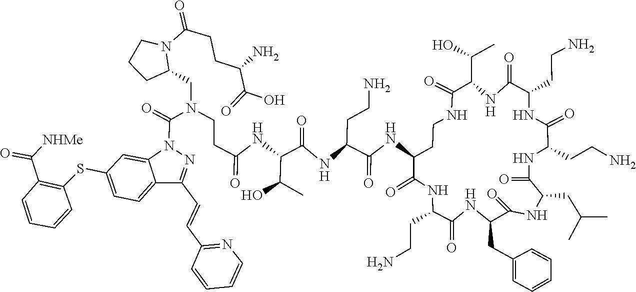 [SMILES: CNC(=O)c1ccccc1Sc1ccc2c(/C=C/c3ccccn3)nn(C(=O)N(CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)C[C@@H]3CCCN3C(=O)CC[C@H](N)C(=O)O)c2c1] |
| 166 | 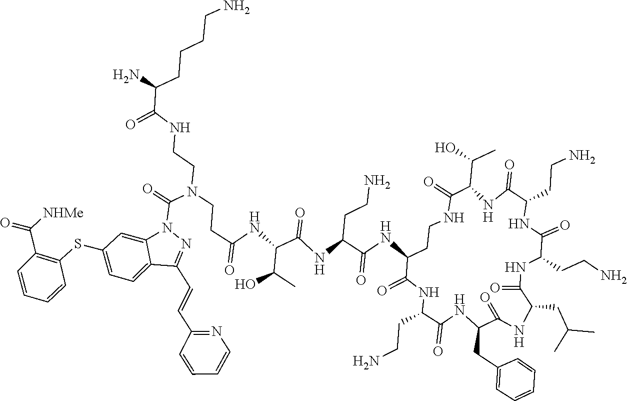 [SMILES: CNC(=O)c1ccccc1Sc1ccc2c(/C=C/c3ccccn3)nn(C(=O)N(CCNC(=O)[C@@H](N)CCCCN)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 167 | 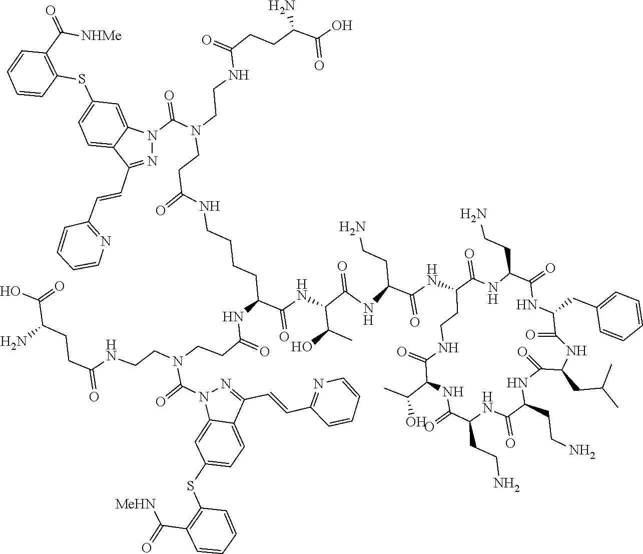 [SMILES: CNC(=O)c1ccccc1Sc1ccc2c(/C=C/c3ccccn3)nn(C(=O)N(CCNC(=O)CC[C@H](N)C(=O)O)CCC(=O)NCCCC[C@H](NC(=O)CCN(CCNC(=O)CC[C@H](N)C(=O)O)C(=O)n3nc(/C=C/c4ccccn4)c4ccc(Sc5ccccc5C(=O)NC)cc43)C(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 168 | 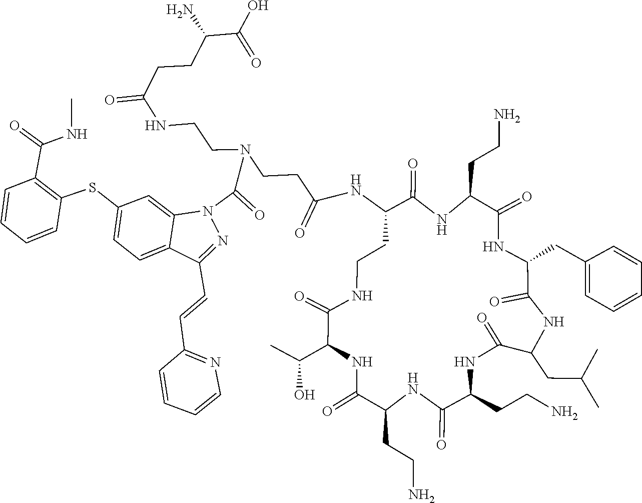 [SMILES: CNC(=O)c1ccccc1Sc1ccc2c(/C=C/c3ccccn3)nn(C(=O)N(CCNC(=O)CC[C@H](N)C(=O)O)CCC(=O)N[C@H]3CCNC(=O)[C@H]([C@@H](C)O)NC(=O)[C@H](CCN)NC(=O)[C@H](CCN)NC(=O)C(CC(C)C)NC(=O)[C@@H](Cc4ccccc4)NC(=O)[C@H](CCN)NC3=O)c2c1] |
| 169 | 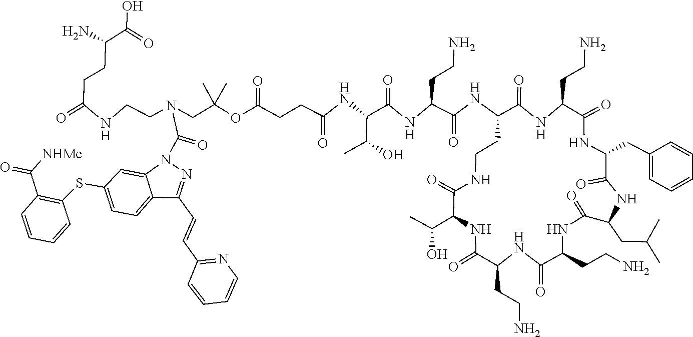 [SMILES: CNC(=O)c1ccccc1Sc1ccc2c(/C=C/c3ccccn3)nn(C(=O)N(CCNC(=O)CC[C@H](N)C(=O)O)CC(C)(C)OC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 170 | 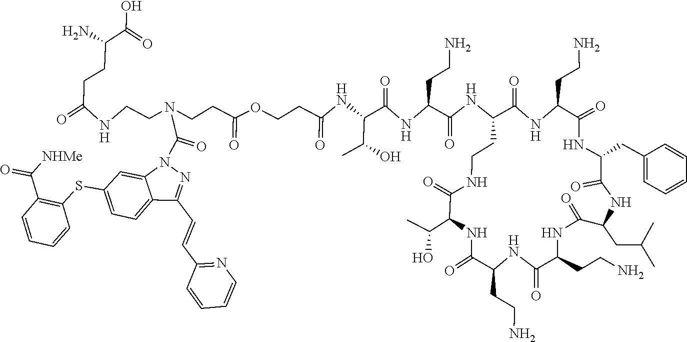 [SMILES: CNC(=O)c1ccccc1Sc1ccc2c(/C=C/c3ccccn3)nn(C(=O)N(CCNC(=O)CC[C@H](N)C(=O)O)CCC(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 171 | 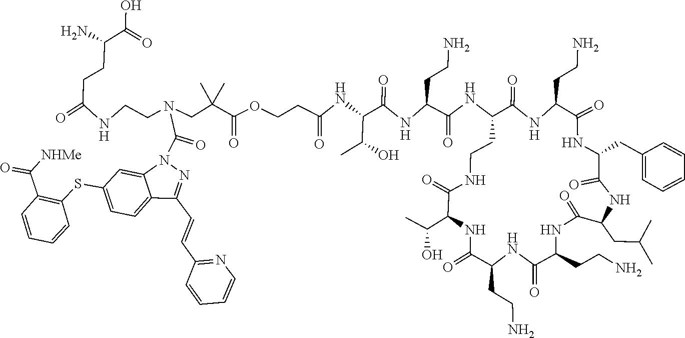 [SMILES: CNC(=O)c1ccccc1Sc1ccc2c(/C=C/c3ccccn3)nn(C(=O)N(CCNC(=O)CC[C@H](N)C(=O)O)CC(C)(C)C(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 172 | 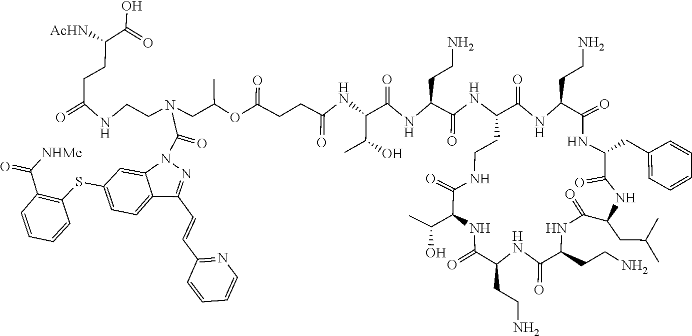 [SMILES: CNC(=O)c1ccccc1Sc1ccc2c(/C=C/c3ccccn3)nn(C(=O)N(CCNC(=O)CC[C@H](NC(C)=O)C(=O)O)CC(C)OC(=O)CCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 173 | 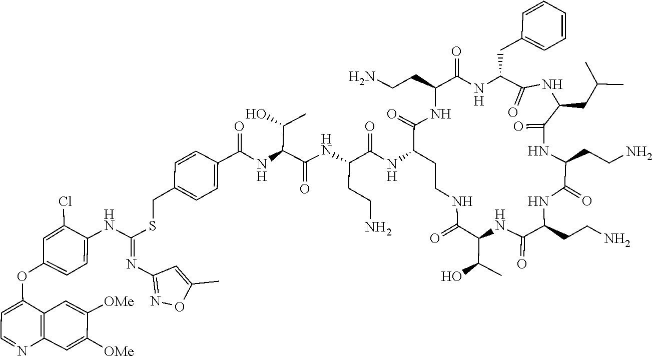 [SMILES: COc1cc2nccc(Oc3ccc(N/C(=N/c4cc(C)on4)SCc4ccc(C(=O)N[C@H](C(=O)N[C@@H](CCN)C(=O)N[C@H]5CCNC(=O)[C@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@@H](C)O)cc4)c(Cl)c3)c2cc1OC] |
| 174 | 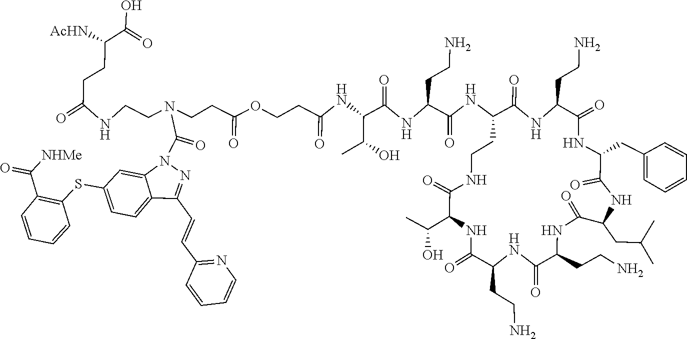 [SMILES: CNC(=O)c1ccccc1Sc1ccc2c(/C=C/c3ccccn3)nn(C(=O)N(CCNC(=O)CC[C@H](NC(C)=O)C(=O)O)CCC(=O)OCCC(=O)N[C@H](C(=O)N[C@@H](CCN)C(=O)N[C@H]3CCNC(=O)[C@H]([C@@H](C)O)NC(=O)[C@H](CCN)NC(=O)[C@H](CCN)NC(=O)[C@H](CC(C)C)NC(=O)[C@@H](Cc4ccccc4)NC(=O)[C@H](CCN)NC3=O)[C@@H](C)O)c2c1] |
| 175 | 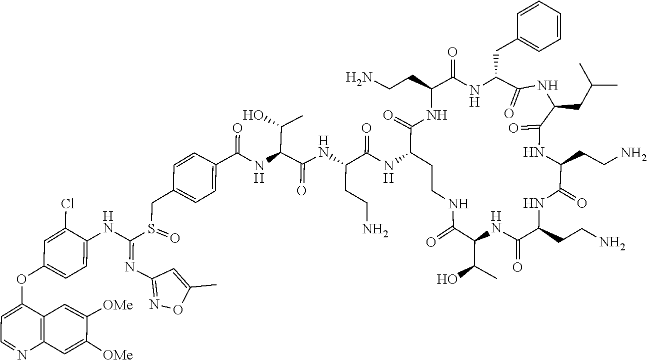 [SMILES: COc1cc2nccc(Oc3ccc(N/C(=N/c4cc(C)on4)S(=O)Cc4ccc(C(=O)N[C@H](C(=O)N[C@@H](CCN)C(=O)N[C@H]5CCNC(=O)[C@H]([C@@H](C)O)NC(=O)[C@H](CCN)NC(=O)[C@H](CCN)NC(=O)[C@H](CC(C)C)NC(=O)[C@@H](Cc6ccccc6)NC(=O)[C@H](CCN)NC5=O)[C@@H](C)O)cc4)c(Cl)c3)c2cc1OC] |

16. The compound of claim 1 selected from the structures below, or a pharmaceutically acceptable salt, solvate, or hydrate thereof:

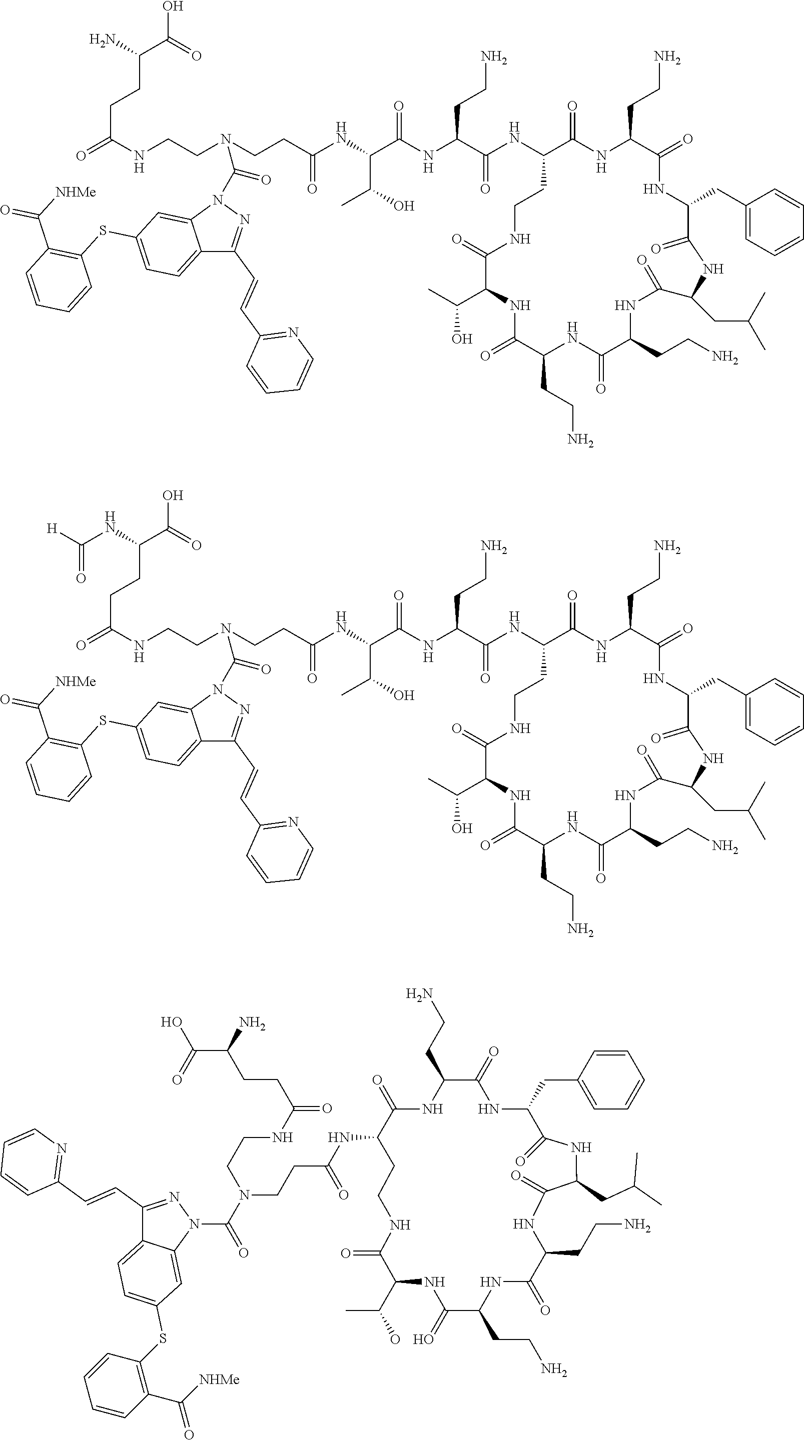

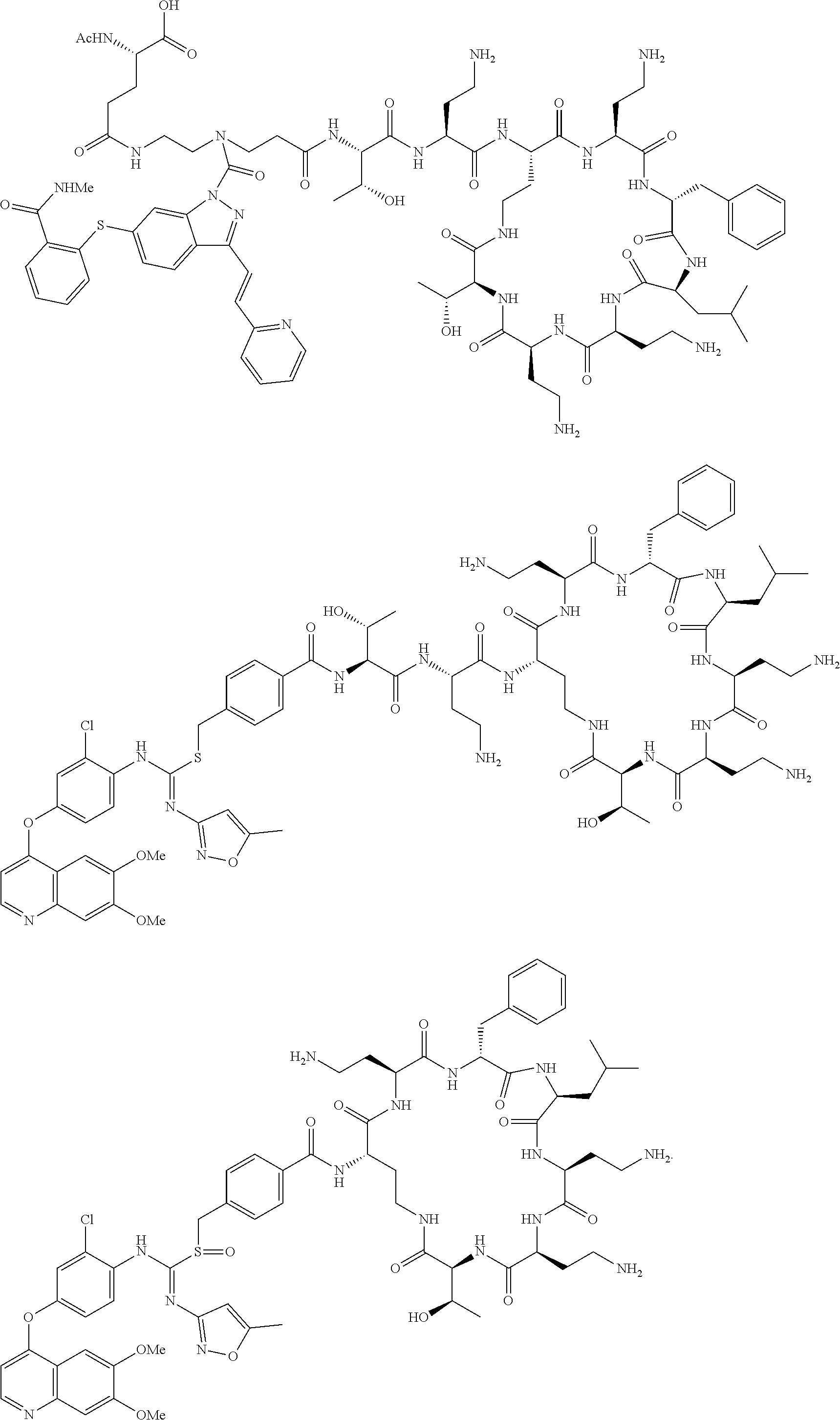

17. The compound of claim 1 selected from the structures below or a pharmaceutical salt, solvate, or hydrate thereof:

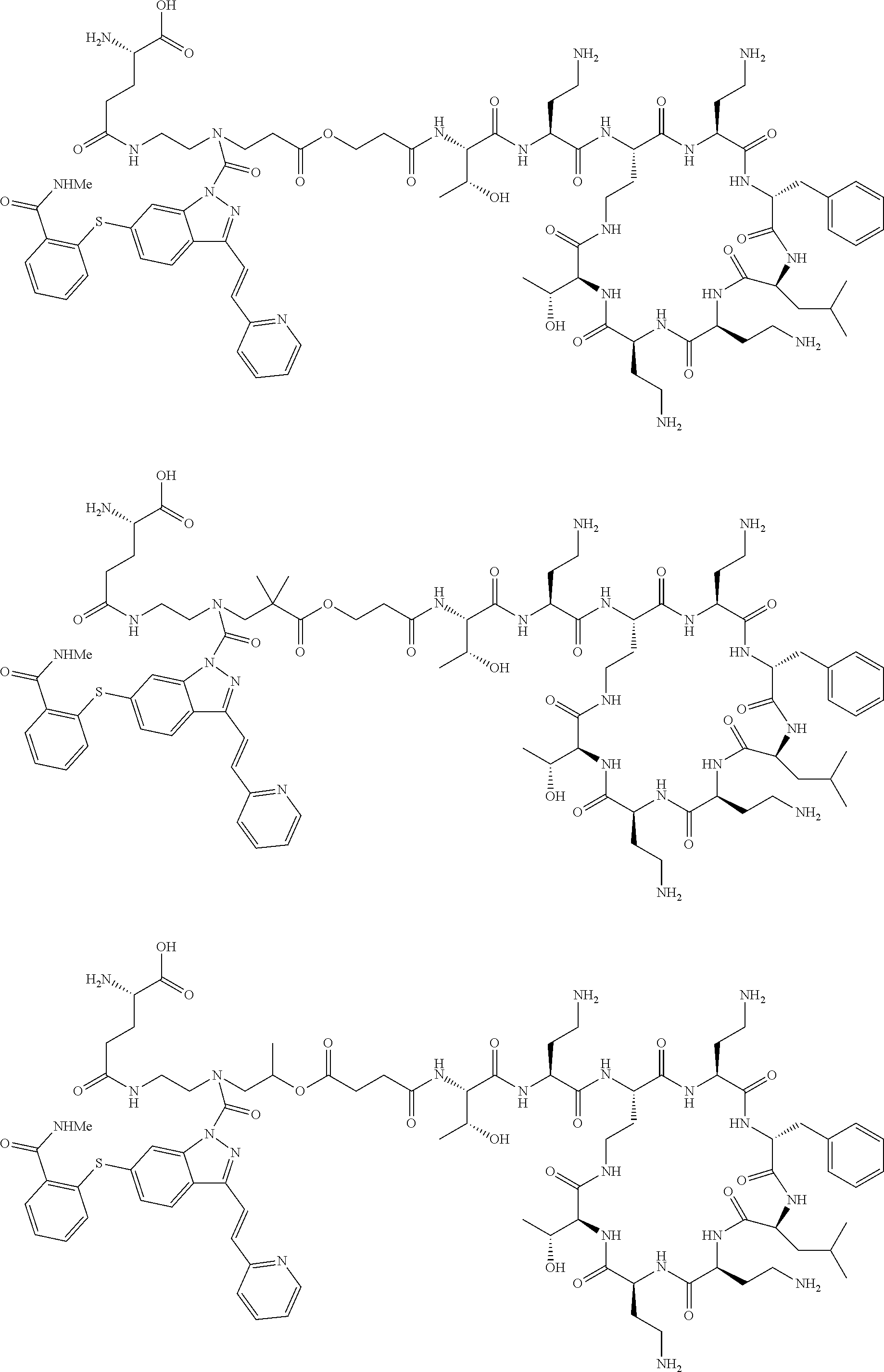

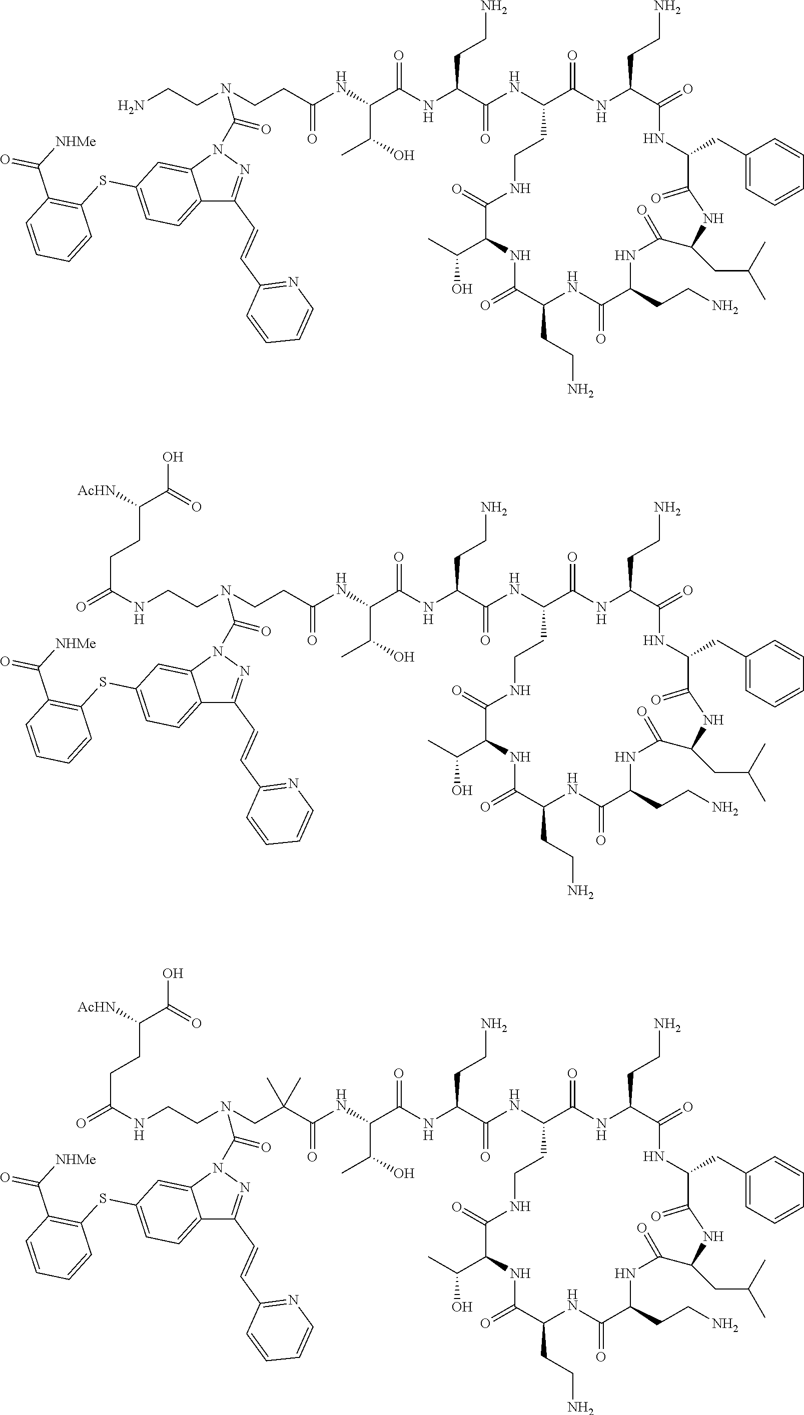

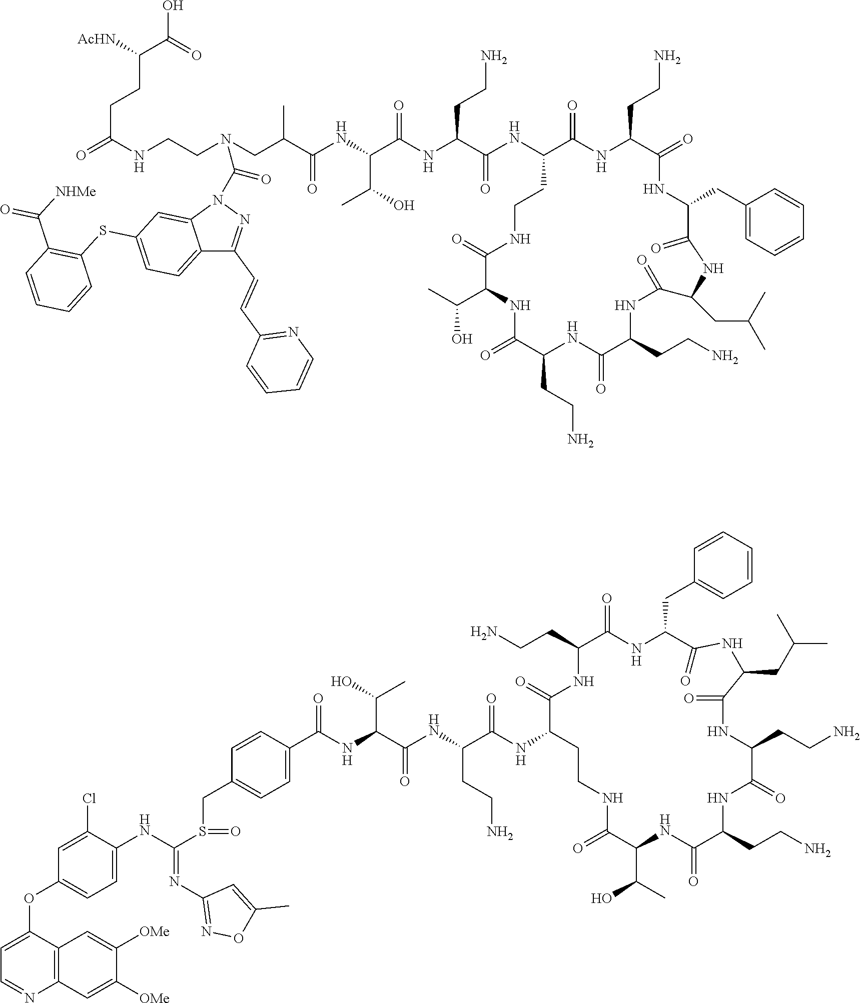

18. The compound of claim 1 or a pharmaceutically acceptable salt, solvate, or hydrate thereof, that exerts a therapeutic effect after administration into a mammal by releasing a bioactive or cytotoxic agent(s) (H)ₙR¹ and/or (H)ₒR².

19. The compound of claim 1, or a pharmaceutically acceptable salt, solvate, or hydrate thereof, possessing anticancer activity against cancerous cells, as determined by inhibition or slowing of cancer cell growth using in vitro cytotoxicity test(s) or assay(s), or by testing of said compounds in animal models for cancer(s).

20. The compound of claim 19, or a pharmaceutically acceptable salt, solvate, or hydrate thereof, wherein a cancer is a renal cancer, or a kidney cancer.

21. The compound of claim 1, or a pharmaceutically acceptable salt, solvate, or hydrate thereof, possessing a reduced cytotoxicity against non-cancerous mammalian cell(s) and wherein, when compared to an agent or drug (H)ₙR¹ and/or (H)ₒR² incorporated into said compound, as determined by in vitro cytotoxicity test(s), such as cell growth inhibition test(s).

22. The compound of claim 20, or a pharmaceutically acceptable salt, solvate, or hydrate thereof, possessing at least about 50% reduced cytotoxicity against non-cancerous mammalian cell(s), when compared to the corresponding agent or drug of formula (H)ₙR¹ and/or (H)ₒR², as determined by in vitro cytotoxicity test(s), such as cell growth inhibition test(s).

23. The compound of claim 1, or a pharmaceutically acceptable salt, solvate, or hydrate thereof, wherein when administered to a mammal, said compound exhibits preferential accumulation in kidneys, with a ratio for its molar concentration in kidneys compared to that in blood of between about 10 and 500.

24. The compound of claim 23, or a pharmaceutically acceptable salt, solvate, or hydrate thereof, wherein when administered to a mammal, said compound exhibits preferential accumulation in kidneys, with a ratio for its molar concentration in kidneys compared to that in blood of at least about 20.

25. The compound of claim 1, or a pharmaceutically acceptable salt, solvate, or hydrate thereof, wherein when administered to a mammal at a dosing (expressed in molar amount) equal to a standard therapeutic dosing (in molar amount) of an agent (H)ₙR¹ and/or (H)ₒR², said compound exhibits about 1.5- to 15-fold higher loading (tissue concentration) of agent (H)ₙR¹ and/or (H)ₒR² in kidneys, as compared to the standard therapeutic dosing of (H)ₙR¹ and/or (H)ₒR².

26. The compound of claim 1, or a pharmaceutically acceptable salt, solvate, or hydrate thereof, wherein when administered to a mammal at a dosing (expressed in molar amount) equal to a standard therapeutic dosing (in molar amount) of an agent(s) (H)ₙR¹ and/or (H)ₒR², said compound exhibits at least 2-fold higher loading (tissue concentration) of agent(s) (H)ₙR¹ and/or (H)ₒR² in kidneys, as compared to the standard therapeutic dosing of (H)ₙR¹ and/or (H)ₒR².

27. The compound of claim 1, or a pharmaceutically acceptable salt, solvate, or hydrate thereof, wherein when administered to a mammal at a dosing (expressed in molar amount) equal to a standard therapeutic dosing (in molar amount) of agent(s) (H)ₙR¹ and/or (H)ₒR², exhibits about 1.5- to 15-fold higher efficacy, as compared to the standard therapeutic dosing of agent(s) (H)ₙR¹ and/or (H)ₒR², with said therapeutic effect determined as a slowed, stopped, or reversed progression of cancer (as determined per changes in a cancer tumor size, and/or by using biochemical biomarkers for cancer monitoring, or similar methods).

28. The compound of claim 27, or a pharmaceutically acceptable salt, solvate, or hydrate thereof, wherein when administered to a mammal at a dosing (expressed in molar amount) equal to a standard therapeutic dosing (in molar amount) of agent(s) (H)ₙR¹ and/or (H)ₒR², exhibits at least 2-fold higher efficacy, as compared to the standard therapeutic dosing of agent(s) (H)ₙR¹ and/or (H)ₒR², with said therapeutic effect determined as a slowed, stopped, or reversed progression of cancer (as determined per changes in a cancer tumor size, and/or by using biochemical biomarkers for cancer monitoring, or similar methods).

29. The compound of claim 1, or a pharmaceutically acceptable salt, solvate, or hydrate thereof, wherein when administered to a mammal at a dosing (expressed in molar amount) equal to a standard therapeutic dosing (in molar amount) of an agent (H)ₙR¹ and/or (H)ₒR², exhibits at least 2-fold reduced rate of adverse effects and/or off-target toxicity manifestation, as compared to the standard therapeutic dosing of (H)ₙR¹ and/or (H)ₒR², as determined by gross observations of a mammal under therapy, a blood cells count, a tissue biopsy, and/or by analysis of biochemical biomarkers, or similar method.

30. A pharmaceutical composition comprising a therapeutically effective amount of a compound of claim 1, or a pharmaceutically acceptable salt, solvate, or hydrate thereof, and a pharmaceutically acceptable carrier.

31. A method treating a cancer in a mammal comprising administering to the mammal a therapeutically effective amount of a compound of claim 1, or a pharmaceutically acceptable salt, solvate, or hydrate thereof, or the pharmaceutical composition of claim 30.

32. The method according to claim 31, wherein the compound, or a pharmaceutically acceptable salt, solvate, or hydrate thereof, or wherein the pharmaceutical composition is administered to the mammal parenterally, transdermally, orally, intranasally, topically, rectally, or via an intra-tumoral administration in a pharmaceutical composition.

33. The method of claim 31, wherein the cancer is renal cell carcinoma or metastatic renal cell carcinoma.
